(19) United States
(12) Patent Application Publication
(10) Pub. No.: US 2007/0066624 A1
(43) Pub. Date: Mar. 22, 2007

(54) Chemokine receptor binding compounds

(75) Inventors: Yuanxi Zhou, Richmond (CA); Elyse Bourque, Langley (CA); Yongbao Zhu, Coquitlam (CA); Ernest J. McEachern, Vancouver (CA); Curtis Harwig, Vancouver (CA); Renato T. Skerlj, Vancouver (CA); Gary J. Bridger, Bellingham, WA (US); Tong-Shuang Li, Langley (CA); Markus Metz, Delta (CA)

Correspondence Address: MORRISON & FOERSTER LLP, 12531 HIGH BLUFF DRIVE, SUITE 100, SAN DIEGO, CA, 92130-2040, US

(73) Assignee: AnorMED, Inc., Langley (CA)

(21) Appl. No.: 11/505,669

(22) Filed: Aug. 16, 2006

Related U.S. Application Data
(60) Provisional application No. 60/708,471, filed on Aug. 16, 2005.

(51) Int. Cl. A61K 31/496; A61K 31/454; C07D 403/14; C07D 403/04

(52) U.S. Cl. 514/254.01; 514/254.05; 514/326; 544/370; 544/373; 546/210

(57) ABSTRACT

The present invention relates to chemokine receptor binding compounds, pharmaceutical compositions and their use. More specifically, the present invention relates to modulators of chemokine receptor activity, preferably modulators of CCR4 or CCR5. In one aspect, these compounds demonstrate protective effects against infection of target cells by a human immunodeficiency virus (HIV).

### CROSS REFERENCE TO RELATED APPLICATIONS

This application claims benefit of U.S. provisional application Ser. No. 60/708,471, filed Aug. 16, 2005, which is incorporated herein in its entirety.

### TECHNICAL FIELD

This invention generally relates to novel compounds, pharmaceutical compositions and their use. More specifically, these novel compounds may be modulators of chemokine receptor activity, preferably modulators of chemokine receptor CCR5, and may further demonstrate protective effects against infection in target cells by a human immunodeficiency virus (HIV). In another aspect, the compounds in the present invention may be useful in the treatment and prevention of various inflammatory and autoimmune diseases.

### BACKGROUND OF THE INVENTION

Approximately 40 human chemokines have been described that function at least in part, by modulating a complex and overlapping set of biological activities important for the movement of lymphoid cells and extravasation and tissue infiltration of leukocytes in response to inciting agents (See, for example: P. Ponath, Exp. Opin. Invest. Drugs, 7:1-18, 1998). These chemotactic cytokines, or chemokines, constitute a family of proteins, approximately 8-10 kDa in size, that are released by a wide variety of cells, to attract macrophages, T cells, eosinophils, basophils, and neutrophils to sites of inflammation and also play a role in the maturation of cells of the immune system. Chemokines appear to share a common structural motif that consists of 4 conserved cysteines involved in maintaining tertiary structure. There are two major subfamilies of chemokines: the “CC” or β-chemokines and the “CXC” or α-chemokines, depending on whether the first two cysteines are separated by a single amino acid, i.e., CXC or are adjacent, i.e., CC.

These chemokines bind specifically to cell-surface receptors belonging to the family of G-protein-coupled seven-transmembrane proteins which are referred to as “chemokine receptors”, and mediate biological activity through these receptors. The chemokine receptor is classified based upon the chemokine that constitutes the receptor's natural ligand. Chemokine receptors of the β-chemokines are designated “CCR”; while those of the α-chemokines are designated “CXCR.” These chemokine receptors include but are not limited to CCR1, CCR2, CCR2A, CCR2B, CCR3, CCR4, CCR5, CCR6, CXCR3 and CXCR4 (see for a complete review, Murphy et al. Pharmacol. Rev. 52(1), 145-176 (2000)).

Chemokines are considered to be principal mediators in the initiation and maintenance of inflammation (see Chemokines in Disease published by Humana Press (1999), Edited by C. Herbert; Murdoch et al. Blood 95, 3032-3043 (2000)). More specifically, chemokines have been found to play an important role in the regulation of endothelial cell function, including proliferation, migration and differentiation during angiogenesis and re-endothelialization after injury (Gupta et al., J. Biolog. Chem., 7:4282-4287, 1998). Both chemokine receptors CXCR4 and CCR5 have been implicated in the etiology of infection by human immunodeficiency virus (HIV).

In most instances, HIV initially binds via its gp120 envelope protein to the CD4 receptor of the target cell. A conformational change appears to take place in the gp120 which results in its subsequent binding to a chemokine receptor, such as CCR5 (Wyatt et al., Science, 280:1884-1888 (1998)). HIV-1 isolates arising subsequently in the infection bind to the CXCR4 chemokine receptor. The observed binding of another related retrovirus, feline immunodeficiency virus, to a chemokine receptor without needing to bind first to the CD4 receptor, suggests that chemokine receptors may be the primordial obligate receptors for immunodeficiency retroviruses.

Following the initial binding by HIV to CD4, virus-cell fusion results, which is mediated by members of the chemokine receptor family, with different members serving as fusion cofactors for macrophage-tropic (M-tropic) and T cell line-tropic (T-tropic) isolates of HIV-1 (Carroll et al., Science, 276: 273-276 1997; Feng et al. Science 272, 872-877 (1996); Bleul et al. Nature 382, 829-833 (1996); Oberlin et al. Nature 382, 833-835 (1996); Cocchi et al. Science 270, 1811-1815 (1995); Dragic et al. Nature 381, 667-673 (1996); Deng et al. Nature 381, 661-666 (1996); Alkhatib et al. Science 272, 1955-1958, (1996)). During the course of infection within a patient, it appears that a majority of HIV particles shift from the M-tropic to the more aggressive pathogenic T-tropic viral phenotype (Miedema et al., Immune. Rev., 140:35 (1994); Blaak et al. Proc. Natl. Acad. Sci. 97, 1269-1274 (2000); Simmonds et al. J. Virol. 70, 8355-8360 (1996); Tersmette et al. J. Virol. 62, 2026-2032, (1988); Connor, R. I., Ho, D. D. J. Virol. 68, 4400-4408 (1994); Schuitemaker et al. J. Virol. 66, 1354-1360 (1992)). The M-tropic viral phenotype correlates with the virus' ability to enter the cell following binding of the CCR5 receptor, while the T-tropic viral phenotype correlates with viral entry into the cell following binding and membrane fusion with the CXCR4 receptor. Clinically, observations suggest that patients who possess genetic mutations in the CCR5 or CXCR4 receptors appear resistant or less susceptible to HIV infection (Liu et al. Cell 86, 367-377 (1996); Samson et al. Nature 382, 722-725 (1996); Michael et al. Nature Med. 3, 338-340 (1997); Michael et al. J. Virol. 72, 6040-6047 (1998); Obrien et al. Lancet 349, 1219 (1997); Zhang et al. AIDS Res. Hum. Retroviruses 13, 1357-1366 (1997); Rana et al. J. Virol. 71, 3219-3227 (1997); Theodorou et al. Lancet 349, 1219-1220 (1997)). Despite the number of chemokine receptors which have been reported to mediate HIV entry into cells, CCR5 and CXCR4 appear to be the only physiologically relevant coreceptors used by a wide variety of primary clinical HIV-1 strains (Zhang et al. J. Virol. 72, 9307-9312 (1998); Zhang et al. J. Virol. 73, 3443-3448 (1999); Simmonds et al. J. Virol. 72, 8453-8457 (1988)). Fusion and entry of T-tropic viruses that use CXCR4 are inhibited by the natural CXC-chemokine stromal cell-derived factor-1 (SDF-1). On the other hand, fusion and entry of M-tropic viruses that use CCR5 are inhibited by the natural CC-chemokines namely, Regulated on Activation Normal T-cell Expressed and Secreted (RANTES or CCL5) and Macrophage Inflammatory proteins (MIP-1 alpha and MIP-1 beta or CCL3 and CCL4, respectively). SDF-1 is known as CXCL12 or Pre B-cell stimulating factor (PBSF).

However, the binding of chemokine receptors to their natural ligands appears to serve a more evolutionary and central role than only as mediators of HIV infection. The binding of the natural ligand, PBSF/SDF-1 to the CXCR4 chemokine receptor provides an important signaling mechanism. CXCR4 or SDF-1 knock-out mice exhibit cerebellar, cardiac and gastrointestinal tract abnormalities and die in utero (Zou et al., Nature, 393:591-594 (1998); Tachibana et al., Nature, 393:591-594 (1998); Nagasawa et al. Nature 382, 635-638 (1996)). CXCR4-deficient mice also display hematopoietic defects (Nagasawa et al. Nature 382, 635-638 (1996)). Furthermore, the migration of CXCR4 expressing leukocytes and hematopoietic progenitors to SDF-1 appears to be important for maintaining B-cell lineage and localization of CD34⁺ progenitor cells in bone marrow (Bleul et al. J. Exp. Med. 187, 753-762 (1998); Viardot et al. Ann. Hematol. 77, 195-197 (1998); Auiti et al. J. Exp. Med. 185, 111-120 (1997); Peled et al. Science 283, 845-848 (1999); Qing et al. Immunity 10, 463-471 (1999); Lataillade et al. Blood 95, 756-768 (1999); Ishii et al. J. Immunol. 163, 3612-3620 (1999); Maekawa et al. Internal Medicine 39, 90-100 (2000); Fedyk et al. J. Leukocyte Biol. 66, 667-673 (1999); Peled et al. Blood 95, 3289-3296 (2000)).

The signal provided by SDF-1 on binding to CXCR4 may also play an important role in tumor cell proliferation and regulation of angiogenesis associated with tumor growth (See “Chemokines and Cancer” published by Humana Press (1999); Edited by B. J. Rollins; Arenburg et al. J. Leukocyte Biol. 62, 554-562 (1997); Moore et al. J. Invest. Med. 46, 113-120 (1998); Moore et al. Trends cardiovasc. Med. 8, 51-58 (1998); Seghal et al. J. Surg. Oncol. 69, 99-104 (1998)). Known angiogenic growth factors VEG-F and bFGF, up-regulated levels of CXCR4 in endothelial cells, and SDF-1 can induce neovascularization in vivo (Salcedo et al. Am. J. Pathol. 154, 1125-1135 (1999)). Furthermore, leukemia cells that express CXCR4 migrate and adhere to lymph nodes and bone marrow stromal cells that express SDF-1 (Burger et al. Blood 94, 3658-3667 (1999); Arai et al. Eur. J. Haematol. 64, 323-332 (2000); Bradstock et al. Leukemia 14, 882-888 (2000)).

The binding of SDF-1 to CXCR4 has also been implicated in the pathogenesis of atherosclerosis (Abi-Younes et al. Circ. Res. 86, 131-138 (2000)), renal allograft rejection (Eitner et al. Transplantation 66, 1551-1557 (1998)), asthma and allergic airway inflammation (Yssel et al. Clinical and Experimental Allergy 28, 104-109 (1998); J. Immunol. 164, 5935-5943 (2000); Gonzalo et al. J. Immunol. 165, 499-508 (2000)), Alzheimer's disease (Xia et al. J. Neurovirology 5, 32-41 (1999)) and arthritis (Nanki et al. J. Immunol. 164, 5010-5014 (2000)).

Platelets have also been shown to secrete the chemokine RANTES upon activation, and that the presence of RANTES on the endothelium promotes the arrest of monocytes on the inflamed endothelium, an important step in atherogenesis as the conversion of macrophages into foam cells in the subendothelium is a central process in atheroma formation (Tan, et al., Expert Opin. Investig. Drugs, 12(11):1765-1776 (2003)). Hence, the inhibition or prevention of the binding of RANTES, directly or indirectly, to the CCR5 receptor could potentially attenuate the development of atherosclerosis. For example, Met_RANTES has also been shown to inhibit the binding of monocytes to the activated endothelium (Tan, et al., supra).

In attempting to better understand the relationship between chemokines and their receptors, recent experiments to block the fusion, entry and replication of HIV via the CXCR4 chemokine receptor were carried out through the use of monoclonal antibodies or small molecules that appear to suggest a useful therapeutic strategy (Schols et al., J. Exp. Med. 186:1383-1388 (1997); Schols et al., Antiviral Research 35:147-156 (1997); Bridger et al. J. Med. Chem. 42, 3971-3981 (1999); Bridger et al. “Bicyclam Derivatives as HIV Inhibitors” in Advances in Antiviral Drug Design Volume 3, p161-229; Published by JAI press (1999); Edited by E. De Clercq). Small molecules, such as bicyclams, appear to specifically bind to CXCR4 and not CCR5 (Donzella et al., Nature Medicine, 4:72-77 (1998)). These experiments demonstrated interference with HIV entry and membrane fusion into the target cell in vitro. More recently, bicyclams were also shown to inhibit fusion and replication of Feline Immunodeficiency Virus (FIV) that uses CXCR4 for entry (Egberink et al. J. Virol. 73, 6346-6352 (1999)). CCR5 blocking agents include monoclonal antibodies, some which selectively block HIV coreceptor activity but not chemokine binding, and chemokine derivatives, such as truncated versions of RANTES, Met-RANTES, and AOP-RANTES and the viral chemokine KSHV vMIP-II, all which block both chemokine and HIV interaction with CCR5 but are not selective (reviewed by Murphy et al. Pharmacol. Rev. 52(1), 145-176 (2000)).

Additional experiments have shown that the bicyclam dose-dependently inhibits binding of 125I-labeled SDF-1 to CXCR4 and the signal transduction (indicated by an increase in intracellular calcium) in response to SDF-1. Thus, the bicyclam also functioned as an antagonist to the signal transduction resulting from the binding of stromal derived factor or SDF-1α, the natural chemokine to CXCR4. Bicyclams also inhibited HIV gp120 (envelope)-induced apoptosis in non-HIV infected cells (Blanco et al. Antimicrobial Agents and Chemother. 44, 51-56 (2000)).

Passive immunization with anti-MIP-1 alpha has been shown to delay the onset and reduce the severity of collagen-induced-arthritis (CIA) in mice, where the CIA model is an established murine model representing human rheumatoid arthritis (Szekanecz, Z., et al., AP, Seminars in Immunology, 15(2003), p.15-21). Other studies have also shown that agents that block the CCR5 receptor may provide a rational approach to the treatment of multiple sclerosis. Administration of anti-MIP-1 alpha antiserum has been shown to prevent CNS infiltration by PBMC in mice with experimental allergic encephalomyelitis, a rodent model of multiple sclerosis (Balashov, K. E., et al., Proc. Natl. Acad. Sci. USA, Vol. 96 (1999), p. 6873-6878). Other studies involving chronic rejection of transplanted hearts or cardiac allograft vasculopathy (CAV) and acute renal allograft rejection have shown that blocking chemokine receptors such as CCR5 may provide unique therapeutic approaches in the treatment or prevention of such diseases (Yun J J, et al., Circulation, 2004, Vol. 109(7), p.932-7, Panzer U., et al., Transplantation, 2004, Vol. 78(9), p.1341-50). For example, antagonism of the chemokine receptors CCR1 and CCR5 with Met-RANTES attenuated CAV development by reducing mononuclear cell recruitment to the transplanted heart. Met-CCL5, an antagonist of CCR1 and CCR5, had been tested and shown to inhibit the growth of breast tumors (Robinson S C. et al, Cancer Res., 2003, Vol. 63(23), p.8360-5).

Chemokines, as indicated above, play an important role and are implicated in a wide variety of human disease such as in autoimmune disease, allograft rejection, infection, allergies, neoplasia, and vascular abnormalities. In addition to its contributory role in HIV infection, the chemokine receptor CCR5 has been associated with diseases such as the inflammatory demyelinating diseases of the central nervous system, including multiple sclerosis and experimental autoimmune encephalomyelitis, rheumatoid arthritis, intestinal inflammation, allograft rejection, asthma, and cardiovascular disease (reviewed in Gerard et al. Natl. Immunol. 2(2), 108-115 (2001) and Luster, A., N. Eng. J. Med., 338 (7), 436-445 (1998)). The CCR5 receptor is expressed on T-lymphocytes, and macrophages and reports of CCR5 on neurons, astrocytes, capillary endothelial cells, epithelium, vascular smooth muscle, and fibroblast have been published. The natural ligands that bind to the CCR5 receptor, in addition to RANTES and MIP-1 alpha/beta, are monocyte chemoattractant protein 2 (MCP-2 or CCL8).

CCR4, together with its ligands, i.e., macrophage derived chemokine (MDC; CCL22) and thymus and activation regulated chemokine (TARC; CCL17) are responsible for recruitment, homing, and education of activated leukocytes. Recently, CCR4 and its ligands have attracted significant attention due to their involvement in mediating various allergic inflammatory conditions such as asthma, and acute dermatitis. It has been shown that studies involving monoclonal antibodies for the CCR4 receptor and its ligand TARC in OVA-induced murine asthma models, and CCR4 antagonists (Chvatchko et al., J. Exp. Med., 191, 1755-1763 (2000); Purandare et al. Biorg. Med. Chem. Lett., 16, 204-207, (2006)) have targeted antagonism of the CCR4 receptor as a mechanism of inhibiting recruitment of activated leukocytes to the lungs, and this provided support for CCR4 antagonism as a potential treatment for diseases such as asthma, and atopic dermatitis.

Also, chemokine receptors, like CCR5 and CCR4, have been known to play a role in T cell involvement in graft versus host diseases (GVHD). Inhibition of such chemokine receptors, by modulating leukocyte trafficking and migration, can be a potential therapeutic mechanism for treating and preventing GVHD. GVHD, after allogeneic stem cell transplantation, is associated with high T cell numbers (C A Wysocki et al., J Immunol. 173, 845-854 (2004); M. Murai et al., J. Clin. Invest. 104 49-57 (1999); 1 Lee et al., J. Exp. Med. 201 1037-1044 (2005); A. Iellem et al., J. Exp. Med. 194 847-853 (2001); M. Jaksch et al., Biology of Blood and Bone Marrow Transplantation 11 280-287 (2005); and U. Duffner et al., Exptl. Hematol. 31 897-902 (2003)). Current therapy uses immunosuppressant drugs, and the current invention contains compounds that would be more selective in effect with reduced toxicity.

U.S. Pat. Nos. 5,583,131; 5,698,546; 5,817,807; 5,021,409; and 6,001,826 which are incorporated herein in their entirety by reference, disclose cyclic compounds that are active against HIV-1 and HIV-2 in in vitro tests. It was subsequently discovered and further disclosed in PCT WO 02/34745 that these compounds exhibit anti-HIV activity by binding to the chemokine receptor CXCR4 and/or CCR5 expressed on the surface of certain cells of the immune system. This competitive binding thereby protects these target cells from infection by HIV which utilize the CXCR4 receptor for entry. In addition, these compounds antagonize the binding, signaling and chemotactic effects of the natural ligand for CXCR4, the chemokine stromal cell-derived factor 1α (SDF-1). Furthermore, these compounds demonstrate protective effects against HIV infection of target cells by binding in vitro to the CCR5 receptor.

Additionally, U.S. Pat. No. 6,365,583 discloses that these cyclic polyamine antiviral agents described in the above-mentioned patents/patent applications have the effect of enhancing production of white blood cells as well as exhibiting antiviral properties. Thus, these agents are useful for controlling the side-effects of chemotherapy, enhancing the success of bone marrow transplantation, enhancing wound healing and burn treatment, as well as combating bacterial infections in leukemia.

More recently, PCT WO 00/56729, PCT WO 02/22600, PCT WO 02/22599, and PCT WO 02/34745 describe a series of heterocyclic compounds that exhibit anti-HIV activity by binding to the chemokine receptors CXCR4 and CCR5 expressed on the surface of certain cells of the immune system. This competitive binding thereby protects these target cells from infection by HIV which utilize the CXCR4 or CCR5 receptors for entry. In addition, these compounds antagonize the binding, signaling and chemotactic effects of the natural ligand for CXCR4, the chemokine stromal cell-derived factor 1α (SDF-1) and/or the natural ligand for CCR5, the chemokine RANTES.

The chemokine receptor, CXCR4 has been found to be associated with the vascularization of the gastrointestinal tract (Tachibana et al., Nature, 393:591-594 (1998)) as well as in hematopoiesis and cerebellar development (Zou et al., Nature, 393:591-594 (1998)). Interference with any of these important functions served by the binding of pre-B-cell growth-stimulating factor/stromal derived factor (PBSF/SDF-1) to the CXCR4 chemokine receptor results in lethal deficiencies in vascular development, hematopoiesis and cardiogenesis. Similarly, fetal cerebellar development appears to rely upon the effective functioning of CXCR4 in neuronal cell migration and patterning in the central nervous system. This G-protein-coupled chemokine receptor appears to play an important role in ensuring the necessary patterns of migration of granule cells in the cerebellar anlage.

Herein, we disclose compounds that have unique chemical attributes and that exhibit protective effects against HIV infection of target cells by binding to chemokine receptor CCR5. In addition, these compounds antagonize the binding, signaling and chemotactic effects of the natural ligand for CCR5, the chemokine RANTES. Further, the disclosed compounds bind to the chemokine receptor CCR4 and thus are useful in treating CCR4-mediated diseases.

Citation of the above documents is not intended as an admission that any of the foregoing is pertinent prior art. All statements as to the date or representation as to the contents of these documents is based on the information available to the applicants and does not constitute any admission as to the correctness of the dates or contents of these documents. Further, all documents referred to throughout this application are hereby incorporated in their entirety by reference herein.

### DISCLOSURE OF THE INVENTION

The present invention provides novel compounds that may modulate chemokine receptors and interfere with the binding of the natural ligand thereto. The compounds of the present invention may be useful as agents demonstrating protective effects on target cells from HIV infection. In another aspect, the present invention provides novel compounds that may be useful for the treatment and prevention of inflammatory and autoimmune diseases. Embodiments of the present invention are compounds that may act as antagonists or agonists of chemokine receptors, which may be useful as agents capable of reconstituting the immune system by increasing the level of CD4⁺ cells; as antagonist agents of apoptosis in immune cells, such as CD8⁺ cells, and neuronal cells; as antagonist agents of migration of human bone marrow B lineage cells to stromal-derived factor 1, as well as other biological activities related to the ability of these compounds to inhibit the binding of chemokines to their receptors.

More particularly, the present invention relates to novel piperidine (or piperazine) derivatives that may bind to chemokine receptors, preferably CCR4 or CCR5 receptors, having formula (1): 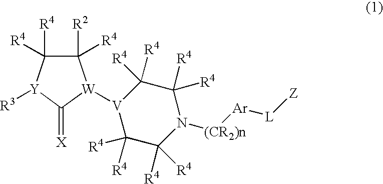
wherein:
V is N or C(R);
W is N or C(R);
X is O, S, NR, N-aryl, N-heteroaryl, N-heterocyclyl, NOR, NCOR, N(CH₂)ₘCOOR, N(CH₂)ₘCONHR, NS(O₂)R, NCN, NNO₂, or CRNO₂, wherein m is 0-3;
Y is O, S, N or C(R);
Z may be absent, H or an optionally substituted alkyl, OR, COOR, C(O)NR₂, carbocyclyl, heterocyclyl, aryl, or heteroaryl;
Ar is an optionally substituted carbocyclyl, heterocyclyl, aryl, or heteroaryl, wherein each of the carbocyclyl and heterocyclyl contains an aryl or heteroaryl ring;
L is absent if Z is absent, or L is a linker between Ar and Z, wherein L is a bond, O, S, N(R), S(O), S(O₂), S(O₂)N(R), C(O), C(O)N(R), N(R)C(O)N(R), N═N, optionally substituted aliphatic C₁₋₆ hydrocarbyl residue optionally containing one or more heteroatoms, or combinations thereof;
R² is an optionally substituted alkyl, alkenyl, alkynyl, carbocyclyl, heterocyclyl, aryl or heteroaryl;
R³ is absent when Y is O or S; or, when Y is N or C(R), R³ is H, NR₂, C(O)NHOR, C(O)N(R)OR, C(O)NR₂, C(O)R, C(O)OR, OR, or an optionally substituted alkyl, carbocyclyl, heterocyclyl, aryl or heteroaryl;
each R and R⁴ is independently H or C₁₋₆ alkyl; and
n is 1-3.

The present invention also provides pharmaceutical compositions comprising a compound having formula (1), and a pharmaceutically acceptable carrier. Furthermore, the present invention provides methods for treating a CCR4- or CCR5-mediated disease, comprising contacting a compound having formula (1), or a pharmaceutical composition thereof, in a system or a subject, thereby treating said CCR4- or CCR5-mediated disease. The system may be a cell, tissue or organ, and said subject is human or animal.

Further, the present invention provides for the use of a compound having formula (1) or a pharmaceutical composition thereof, for treating a CCR4- or CCR5-mediated disease; or for the manufacture of a medicament for treating a CCR4- or CCR5-mediated disease. 28Examples of CCR5-mediated diseases that may be treated using the compounds of the present invention include but are not limited to HIV, an inflammatory demyelinating disease of the central nervous system, an autoimmune disease, multiple sclerosis, experimental autoimmune encephalomyelitis, psoriatic or rheumatoid arthritis, intestinal inflammation, allograft rejection, asthma, cardiovascular disease, atherosclerosis, allergic disease, allergic rhinitis, dermatitis, conjunctivitis, hypersensitivity lung disease, hypersensitivity pneumonitis, eosinophilic pneumonia, delayed-type hypersensitivity, interstitial lung disease (ILD), idiopathic pulmonary fibrosis, ILD associated with rheumatoid arthritis, systemic lupus erythematosus, ankylosing spondylitis, systemic sclerosis, Sjogren's syndrome, polymyositis, dermatomyositis, systemic anaphylaxis, myastenia gravis, juvenile onset diabetes, glomerulonephritis, autoimmune thyroiditis, graft rejection, allograft rejection, graft-versus-host disease, inflammatory bowel disease, Crohn's disease, ulcerative colitis, spondyloarthropathy, scleroderma; psoriasis, inflammatory dermatosis, dermatitis, eczema, atopic dermatitis, allergic contact dermatitis, urticaria, vasculitis, eosinophilic myotis, eosiniphilic fasciitis, tumor or cancer. In one embodiment, compounds having formula (1) are used for treating HIV.

Examples of CCR4-mediated diseases that may be treated using the compounds of the present invention include but are not limited to allergic inflammatory conditions, such as asthma, acute or atopic dermatitis, or graft versus host disease.

The compounds of formula (1) may form hydrates or solvates, and may be in any stereoisomeric forms and mixtures of stereoisomeric forms thereof. Racemate compounds may be separated into individual isomers using known separation and purification methods. Individual optical isomers and a mixture thereof, are included in the scope of the present invention.

### Definitions

As used herein, “hydrocarbyl residue” refers to a residue which contains only carbon and hydrogen. The residue may be aliphatic or aromatic, straight-chain, cyclic, branched, saturated or unsaturated. The hydrocarbyl residue, when so stated however, may contain heteroatoms over and above the carbon and hydrogen members of the substituent residues within the “backbone” of the hydrocarbyl residue.

As used herein, the term “alkyl,” “alkenyl” and “alkynyl” include straight- and branched-chain monovalent substituents. Examples include methyl, ethyl, isobutyl, 2-propenyl, 3-butynyl, and the like. Typically, the alkyl, alkenyl and alkynyl substituents contain 1-10C (alkyl) or 2-10C (alkenyl or alkynyl). Preferably they contain 1-6C (alkyl) or 2-6C (alkenyl or alkynyl). Heteroalkyl, heteroalkenyl and heteroalkynyl are similarly defined but may contain 1-5, preferably 1-2, O, S or N heteroatoms or combinations thereof within the backbone residue.

As used herein, the term “carbocycle” or “carbocyclyl” refers to a cyclic compound containing only carbon atoms in the ring, whereas a “heterocycle” or “heterocyclyl” refers to a cyclic compound comprising a heteroatom. The carbocyclic and heterocyclic structures encompass compounds having monocyclic, bicyclic or multiple ring systems. The carbocyclic or heterocyclic groups may be aliphatic or, fused bicyclic or multiple cyclic rings may also have one or more aromatic or heteroaromatic group. Further, carbocyclyl or heterocyclyl groups may contain a spiro ring, wherein a central carbon atom is a member of two different rings.

As used herein, the term “aryl” refers to a polyunsaturated, typically aromatic hydrocarbon substituent, whereas a “heteroaryl” or “heteroaromatic” refer to an aromatic ring containing a heteroatom. The aryl and heteroaryl structures encompass compounds having monocyclic, bicyclic or multiple ring systems. As used herein, the term “heteroatom” refers to any atom that is not carbon or hydrogen, such as nitrogen, oxygen or sulfur.

When the compounds of Formula 1 contain one or more chiral centers, the invention includes optically pure forms as well as mixtures of stereoisomers or enantiomers.

“Halogen” refers to halogen substituents, such as fluoro, chloro, or bromo.

As used herein, the terms “modulators and/or modulation” encompass modulating activity in all types and subtypes of CCR5 receptors of a target cell, in any tissues of a particular patient where they are found, and in any cell components comprising those tissues that the target cell may be located. For example, the terms “modulators and/or modulation” encompass antagonist/antagonism, agonist/agonism, partial antagonist/partial antagonism, and or partial agonist/partial agonism, i.e., inhibitors, and activators.

As used herein, the term “therapeutically effective amount” means the amount of a compound that will elicit the biological or medical response of a cell, tissue, organ, system, animal or human that is being sought by the researcher, veterinarian, medical doctor, or other clinician.

### MODES OF CARRYING OUT THE INVENTION

In one aspect, the invention provides compounds having formula (1) as described above, which may be chemokine modulators of chemokine receptors.

In more detail, the compounds may bind chemokine receptors and interfere with the binding of the natural ligand thereto, and may demonstrate protective effects on target cells from HIV infection. The compounds may be useful as antagonists or agonists of chemokine receptors, and are thus capable of reconstituting the immune system by increasing the level of CD4⁺ cells; as antagonist agents of apoptosis in immune cells, such as CD8⁺ cells, and neuronal cells; as antagonist agents of migration of human bone marrow B lineage cells to stromal-derived factor 1, as well as other biological activities related to the ability of these compounds to inhibit the binding of chemokines to their receptors.

Chemokine antagonists that interfere in the binding of a chemokine to its receptor are useful to reconstitute the immune system by increasing the level of CD4⁺ cells (Biard-Piechaczyk, et al., Immunol. Lett., 70: 1-3 (1999)); as antagonist agents of apoptosis in immune cells, such as CD8⁺ cells (Herbin, et al., Nature 395:189-193, (1998)), and as antagonist agents of apoptosis in neuronal cells (Ohagen et al., J. of Virol., 73:897-906, (1999); and Hesselgesser, et al., Curr. Biol. 8:595-598, (1998)). Chemokine receptor antagonist agents also inhibit the migration of human bone marrow B lineage cells to stromal-derived factor 1 (See, e.g., E. Fedyk, et al., J. of Leukocyte Biol., 66:667-783, (1999)).

The invention includes pharmaceutical compositions comprising a therapeutically effective amount of a compound of Formula 1 along with at least one excipient, and methods of treating diseases of the human body or the bodies of other mammals with such compositions. In one aspect, the invention provides a method for blocking or interfering with the binding by a chemokine receptor with its natural ligand, comprising contacting of the chemokine receptor with an effective amount of the compound according to Formula 1. The present invention also provides methods of protecting target cells possessing chemokine receptors, which binding to a pathogenic agent results in disease or pathology, comprising administering to a mammalian subject a pharmaceutical composition comprising a therapeutically effective amount of the compound according to Formula 1. The invention includes the use of a compound of Formula 1 in the manufacture of a medicament for the treatment of a disease in which blocking or interfering with binding of a chemokine receptor with its natural ligand is advantageous. The compound is formulated into a composition in an amount corresponding to a therapeutically effective amount of a compound of Formula 1.

The Invention Compounds

The invention compounds are described generally by formula (1). 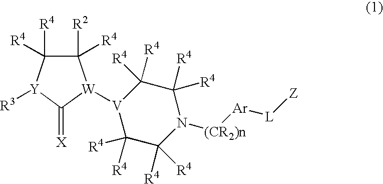
wherein:
V is N or C(R);
W is N or C(R);
X is O, S, NR, N-aryl, N-heteroaryl, N-heterocyclyl, NOR, NCOR, N(CH₂)ₘCOOR, N(CH₂)ₘCONHR, NS(O₂)R, NCN, NNO₂, or CRNO₂, wherein m is 0-3;
Y is O, S, N or C(R);
Z may be absent, H or an optionally substituted alkyl, OR, COOR, C(O)NR₂, carbocyclyl, heterocyclyl, aryl, or heteroaryl;
Ar is an optionally substituted carbocyclyl, heterocyclyl, aryl, or heteroaryl, wherein each of the carbocyclyl and heterocyclyl contains an aryl or heteroaryl ring;
L is absent if Z is absent, or L is a linker between Ar and Z, wherein L is a bond, O, S, N(R), S(O), S(O₂), S(O₂)N(R), C(O), C(O)N(R), N(R)C(O)N(R), N═N, optionally substituted aliphatic C₁₋₆ hydrocarbyl residue optionally containing one or more heteroatoms, or combinations thereof;
R² is an optionally substituted alkyl, alkenyl, alkynyl, carbocyclyl, heterocyclyl, aryl or heteroaryl;
R³ is absent when Y is O or S; or, when Y is N or C(R), R³ is H, NR₂, C(O)NHOR, C(O)N(R)OR, C(O)NR₂, C(O)R, C(O)OR, OR, or an optionally substituted alkyl, carbocyclyl, heterocyclyl, aryl or heteroaryl;
each R and R⁴ is independently H or C₁₋₆ alkyl; and
n is 1-3.

In one aspect of the above Formula 1, V is CH.

In another aspect, W is N.

In yet another aspect X is O, S, N-pyridyl, N-phenyl, NOR or NCH₂COOR.

In another aspect of Formula 1, Y is N, O or C(R). In a preferred embodiment, when Y is C(R), R is H or methyl.

In another aspect, Z is an optionally substituted alkyl, alkoxy, cycloalkyl, phenyl, benzyl, pyridinyl, pyrimidinyl, tetrahydropyranyl, piperidinyl, piperazinyl, dihydroisoindolonyl, dihydroindolonyl, or benzodioxolyl. Preferably, Z is optionally substituted with one or more (preferably one or two) alkyl, carbocyclyl, heterocyclyl, aryl, heteroaryl, alkenyl, alkynyl, halogen, CN, CHO, CF₃, OCF₃, NO₂, R⁵, NRR⁵, OR⁵, N(R)C(O)R⁵, N(R)C(O)CF₃, N(R)S(O₂)R⁵, N(R)S(O₂)NRR⁵, N(R)C(O)NRR⁵, SO₃R, C(O)NRR⁵, C(O)N(OC₁₋₆ alkyl)R, C(O)R⁵, OS(O₂)R, OC(O)NRR⁵, OC(O)R⁵, COOR⁵, SR⁵, S(O)R⁵, S(O₂)R⁵, C(R)═NOH, C(R)═NO(C₁₋₆ alkyl), C(R)═N(C₁₋₆ alkyl), (EC₁₋₄ linker)R⁵, (C₁₋₄ linker)Cl, (C₁₋₄ linker)CN, (C₁₋₄ linker)CF₃, (C₁₋₄ linker)OCF₃, (C₁₋₄ linker)NRR⁵, (C₁₋₄ linker)OR⁵, (C₁₋₄ linker)N(R)C(O)R⁵, (C₁₋₄ linker)N(R)C(O)CF₃, (C₁₋₄ linker)N(R)S(O₂)R⁵, (C₁₋₄ linker)N(R)S(O₂)NRR⁵, (C₁₋₄ linker)N(R)C(O)NRR⁵, (C₁₋₄ linker)SO₃R, (C₁₋₄ linker)C(O)NRR⁵, (C₁₋₄ linker)C(O)N(OC₁₋₆ alkyl)R, (C₁₋₄ linker)C(O)R⁵, (C₁₋₄ linker)OS(O₂)R, (C₁₋₄ linker)OC(O)NRR⁵, (C₁₋₄ linker)OC(O)R⁵, (C₁₋₄ linker)COOR⁵, (C₁₋₄ linker)SR⁵, (C₁₋₄ linker)S(O)R⁵, (C₁₋₄ linker)S(O₂)R⁵, (C₁₋₄ linker)C(R)═NOH, (C₁₋₄ linker)C(R)═NO(C₁₋₆ alkyl), (EC₁₋₄ linker)CN, (EC₁₋₄ linker)CF₃, (EC₁₋₄ linker)NRR⁵, (EC₁₋₄ linker)OR⁵, (EC₁₋₄ linker)N(R)C(O)R⁵, (EC₁₋₄ linker)N(R)C(O)CF₃, (EC₁₋₄ linker)N(R)S(O₂)R⁵, (EC₁₋₄ linker)N(R)S(O₂)NRR⁵, (EC₁₋₄ linker)N(R)C(O)NRR⁵, (EC₁₋₄ linker)C(O)NRR⁵, (EC₁₋₄ linker)R⁵, (EC₁₋₄ linker)C(O)N(OC₁₋₆ alkyl)R, (EC₁₋₄ linker)C(O)R⁵, (EC₁₋₄ linker)OS(O₂)R, (EC₁₋₄ linker)OC(O)NRR⁵, (EC₁₋₄ linker)OC(O)R⁵, (EC₁₋₄ linker)COOR⁵, (EC₁₋₄ linker)SR⁵, (EC₁₋₄ linker)S(O)R⁵, (EC₁₋₄ linker)S(O₂)R⁵, (EC₁₋₄ linker)C(R)═NOH, (EC₁₋₄ linker)C(R)═NO(C₁₋₆ alkyl), or (EC₁₋₄ linker)C(R)═N(C₁₋₆ alkyl), wherein E is O, S, or N(R), wherein R⁵ is H or alkyl, carbocyclyl, heterocyclyl, aryl or heteroaryl, each of which is optionally substituted by one or more of C₁₋₆ alkyl, OR, NR₂, NR(C₁₋₆ alkyl), halogen, CN, CF₃, OCF₃, N(R)C(O)(C₁₋₆ alkyl), (C₁₋₄ linker)COOR, (C₁₋₄ linker)CONHR, C(O)NH₂, C(O)NR(C₁₋₆ alkyl), C(O)N(C₁₋₆ alkyl)₂, C(O)R, COOR, OC(O)R, SR, S(Oₚ)NH₂, S(Oₚ)NR(C₁₋₆ alkyl), N(R)S(O)ₚ(C₁₋₆ alkyl) or SOₚ(C₁₋₆ alkyl) where p is 1 or 2; and wherein C₁₋₄ linker is alkyl, alkenyl or alkynyl.

More preferably, Z is unsubstituted or is optionally substituted with one or two alkyl, CN, halogen, tetrazolyl, OH, COOH, COCOOH, C(O)NH₂, CH═NOH, NHSO₂NR₂, NHSO₂NHR, NH₂, NHCOR, SO₃H, OR, C(O)NHR, C(O)NHOR, C(O)NR₂, NHSO₂R, OC(O)R, (C₁₋₄ linker)COOH, (C₁₋₄ linker)C(O)NHR, (C₁₋₄ linker)C(O)NHOR, (C₁₋₄ linker)OH, (C₁₋₄ linker)NHSO₂NR₂, (C₁₋₄ linker)NHSO₂R, (C₁₋₄ linker)OC(O)R, NH(C₁₋₄ linker)COOH, (C₁₋₄ linker)NH2, S(C₁₋₄ linker)C(O)NHR, S(C₁₋₄ linker)COOH, S(C₁₋₄ linker)C(O)NHOR, O(C₁₋₄ linker)C(O)NHR, O(C₁₋₄ linker)COOH or O(C₁₋₄ linker)C(O)NHOR, wherein C₁₋₄ linker is alkyl, alkenyl or alkynyl.

In another aspect, Ar of Formula 1 is selected from the group consisting of phenyl, quinolyl, tetrahydroquinolyl, dihydroisoindolyl, thiazolyl, pyrimidinyl, pyridyl, benzimidazolyl, imidazolyl, pyrrolyl, thienyl, benzofuranylyl, indanonyl, pyrazolyl, benzo[1,3]dioxolyl, pyranyl, imidazo[1,2-a]pyridinyl, spirobenzodioxolecyclohexyl, and dihydro-isoindolonyl, dihydroindolonyl, and wherein Ar is optionally substituted. Preferably, Ar is an optionally substituted phenyl, quinolyl, tetrahydroquinolyl, dihydroisoindolonyl, thiazolyl, pyrimidinyl, pyridyl, pyrazolyl, benzo[1,3]dioxolyl, imidazo[1,2-a]pyridinyl, spirobenzodioxolecyclohexyl, or dihydro-isoindol-1-onyl. More preferably, Ar is unsubstituted or is optionally substituted with one or more of alkyl, carbocyclyl, heterocyclyl, aryl, heteroaryl, alkenyl, alkynyl, R⁵, OR⁵, NHR⁵, N(R⁵)₂, halogen, CN, CF₃, OCF₃, N(R)C(O)(R⁵), C(O)NRR⁵, C(O)N(R⁵)₂, C(O)R⁵, C(O)OR⁵, OC(O)R⁵, SR⁵, S(O)ₚR⁵, S(O)ₚNRR⁵, or N(R)S(O)ₚR⁵; wherein R⁵ is H or alkyl, carbocyclyl, heterocyclyl, aryl or heteroaryl ring, each of which is optionally substituted by one or more of C₁₋₆ alkyl, OR, NR₂, NR(C₁₋₆ alkyl), halogen, CN, CF₃, OCF₃, N(R)C(O)(C₁₋₆ alkyl), (C₁₋₄ linker)COOR, (C₁₋₄ linker)CONHR, C(O)NH₂, C(O)NR(C₁₋₆ alkyl), C(O)N(C₁₋₆ alkyl)₂, C(O)R, COOR, OC(O)R, SR, S(Oₚ)NH₂, S(Oₚ)NR(C₁₋₆ alkyl), N(R)S(O)ₚ(C₁₋₆ alkyl) or SOₚ(C₁₋₆ alkyl) where p is 1 or 2, wherein C₁₋₄ linker is alkyl, alkenyl or alkynyl. More preferably, Ar is unsubstituted or is optionally substituted with one or two C₁₋₆ alkyl, OR, CN, or halogen.

In another aspect, L is absent, a bond, CH(R), C(R₂), O, N(R), S, S(O), S(O₂), S(O₂)NH, NHC(O)NH, C(O), N(R)C(O), N(R)S(Oₚ), N(R)C(O)N(R), C(O)N(R), OC(O)N(R), OC(O), C(R)═C(R), C≡C, C(R)═N, N═C(R), N═N, (C₁₋₄ linker)O, (C₁₋₄ linker)N(R), (C₁₋₄ linker)S, (C₁₋₄ linker)S(Oₚ), (C₁₋₄ linker)C(O), (C₁₋₄ linker)N(R)C(O), (C₁₋₄ linker)N(R)S(Oₚ), (C₁₋₄ linker)N(R)C(O)N(R), (C₁₋₄ linker)C(O)N(R), (C₁₋₄ linker)OC(O)N(R), (C₁₋₄ linker)OC(O), (C₁₋₄ linker)N═C(R), (C₁₋₄ linker)N═N, or (C₁₋₄ linker)C(R)═N where p is 1 or 2, wherein the C₁₋₄ linker is alkyl, alkenyl or alkynyl. Preferably, L is a bond, O, CH₂, CHMe, CMe₂, NMe, S, NH, C(O), C(O)NH, S(O₂)NH, NHC(O)NH, or (C₁₋₄ linker)NHC(O)NH.

In yet another aspect, R² is an optionally substituted alkyl, alkenyl, alkynyl, phenyl, thienyl, or pyridyl. Preferably, R² is unsubstituted or is optionally substituted with 1-4 substituents selected from the group consisting of alkyl, alkenyl, alkynyl, OR⁵, NHR⁵, N(R⁵)₂, halogen, CN, NO₂, CF₃, OCF₃, N(R)C(O)(R⁵), C(O)NRR⁵, C(O)N(R⁵)₂, C(O)R⁵, C(O)OR⁵, OC(O)R⁵, SR⁵, S(O)ₚR⁵, S(O)ₚNRR⁵, and N(R)S(O)ₚR⁵ where p is 1 or 2; wherein R⁵ is H or alkyl, carbocyclyl, heterocyclyl, aryl or heteroaryl ring, each of which is optionally substituted by one or more of C₁₋₆ alkyl, OR, NR₂, NR(C₁₋₆ alkyl), halogen, CN, CF₃, OCF₃, N(R)C(O)(C₁₋₆ alkyl), (C₁₋₄ linker)COOR, (C₁₋₄ linker)CONHR, C(O)NH₂, C(O)NR(C₁₋₆ alkyl), C(O)N(C₁₋₆ alkyl)₂, C(O)R, COOR, OC(O)R, SR, S(Oₚ)NH₂, S(Oₚ)NR(C₁₋₆ alkyl), N(R)S(O)ₚ(C₁₋₆ alkyl) or SOₚ(C₁₋₆ alkyl) where p is 1 or 2, wherein C₁₋₄ linker is alkyl, alkenyl or alkynyl. Preferably, R² is unsubstituted or is optionally substituted with 1-2 C₁₋₆ alkyl or halo. When R² is phenyl, it is preferably unsubstituted or has one or two substituents selected from chloro, fluoro, bromo or methyl. If one substituent is present on the phenyl, preferably the substituent is at the 3 position of phenyl, and if two substituents are present, preferably it is at the 2 and 5 positions of phenyl.

In another aspect, R³ is H, NR₂, C(O)NHOR, C(O)NROR, C(O)NR₂, C(O)R, C(O)OR, OR, cyclopropyl, cyclobutyl, cyclopentyl, cyclohexyl, cycloheptyl, tetrahydropyranyl, tetrahydrothiopyranyl, tetrahydrofuranyl, morpholinyl, pyridyl, piperidinyl, imidazolyl, furanyl, tetrazolyl, pyrimidinyl, piperazinyl, thiazolyl, thienyl, C₁₋₆ alkyl, [1,3,4]-oxadiazolyl, bicyclo[4.2.0]octa-1,3,5-triene, oxa-bicyclo[3.2.1 ]octyl, dioxy-hexahydro-1-λ⁶-thiopyranyl or a phenyl that is optionally fused to a 5-6 membered heterocyclic ring, wherein each R³ may be optionally substituted. Preferably, R³ is unsubstituted or is H or an optionally substituted C₁₋₆ alkyl, NR₂, C(O)NHOR, C(O)N(R)OR, C(O)NR₂, C(O)R, C(O)OR, OR, phenyl, pyrimidinyl, piperazinyl, pyridyl, thiazolyl, thienyl, cyclopropyl, cyclopentyl, cyclohexyl, piperidinyl, tetrazole, tetrahydropyranyl, tetrahydrothiopyranyl, tetrahydrofuranyl, dioxy-hexahydro-1-λ⁶-thiopyranyl, or oxa-bicyclo[3.2.1]oct-3-yl. More preferably, R³ is unsubstituted or is optionally substituted with alkyl, aryl, heteroaryl, heterocyclic ring, alkenyl, alkynyl, halogen, CN, CF₃, OCF₃, NO₂, R⁵, NRR⁵, OR⁵, N(R)C(O)R⁵, N(R)C(O)CF₃, N(R)S(O₂)R⁵, N(R)C(O)NR₂, C(O)NRR⁵, C(O)N(OC₁₋₆ alkyl)R, C(O)R, OS(O₂)R, OC(O)NR₂, OC(O)R⁵, COOR⁵, SR⁵, S(O)R⁵, S(O₂)R⁵, (C₁₋₄ linker)R⁵, (C₁₋₄ linker)NHC(O)R, (C₁₋₄ linker)C(O)NHR or (C₁₋₄ linker)C(O)OR; wherein the C₁₋₄ linker is alkyl, alkenyl or alkynyl; wherein R⁵ is H or alkyl, carbocyclyl, heterocyclyl, aryl or heteroaryl ring, each of which is optionally substituted by one or more of C₁₋₆ alkyl, OR, NR₂, NR(C₁₋₆ alkyl), halogen, CN, CF₃, OCF₃, N(R)C(O)(C₁₋₆ alkyl), (C₁₋₄ linker)COOR, (C₁₋₄ linker)CONHR, C(O)NH₂, C(O)NR(C₁₋₆ alkyl), C(O)N(C₁₋₆ alkyl)₂, C(O)R, COOR, OC(O)R, SR, S(Oₚ)NH₂, S(Oₚ)NR(C₁₋₆ alkyl), N(R)S(O)ₚ(C₁₋₆ alkyl) or SOₚ(C₁₋₆ alkyl) where p is 1 or 2. More preferably, R³ is unsubstituted or is optionally substituted with halogen, OR, COOR, alkyl, carbocyclyl, heterocyclyl, aryl, or heteroaryl, wherein each substituent may be optionally substituted.

In another aspect, R⁴ is H.

In another aspect, n is 1.

Preferably, the variables of compounds of Formula (1) are defined as follows:
V is CH;
W is N;
X is O;
Y is N, O or C(R), wherein R is H or C₁₋₆ alkyl;
Z is an optionally substituted alkyl, alkoxy, cycloalkyl, phenyl, benzyl, pyridinyl, pyrimidinyl, tetrahydropyranyl, piperidinyl, piperazinyl, dihydroisoindolonyl, dihydroindolonyl, or benzodioxolyl;
Ar is an optionally substituted phenyl, quinolyl, tetrahydroquinolyl, dihydroisoindolyl, thiazolyl, pyrimidinyl, pyridyl, pyrazolyl, benzo[1,3]dioxolyl, imidazo[1,2-a]pyridinyl, spirobenzodioxolecyclohexyl, or dihydro-isoindolonyl, wherein Ar is optionally substituted with one or two C₁₋₆ alkyl, OR, CN, or halogen;
L is a bond, O, CH₂, CHMe, CMe₂, NMe, S, NH, C(O), C(O)NH, S(O₂)NH, NHC(O)NH, or (C₁₋₄ linker)NHC(O)NH, wherein C₁₋₄ linker is alkyl, alkenyl or alkynyl;
R³ is H or an optionally substituted C₁₋₆ alkyl, NR₂, C(O)NHOR, C(O)N(R)OR, C(O)NR₂, C(O)R, C(O)OR, OR, phenyl, pyrimidinyl, piperazinyl, pyridyl, thiazolyl, thienyl, cyclopropyl, cyclopentyl, cyclohexyl, piperidinyl, tetrazole, tetrahydropyranyl, tetrahydrothiopyranyl, tetrahydrofuranyl, dioxy-hexahydro-1-λ⁶-thiopyranyl, or oxa-bicyclo[3.2.1]oct-3-yl, wherein R³ is optionally substituted with halogen, OR, COOR, alkyl, carbocyclyl, heterocyclyl, aryl, or heteroaryl, wherein each substituent may be optionally substituted;
R² is alkyl, alkenyl or alkynyl, phenyl, thienyl or pyridyl substituted with one or two halogen or alkyl;
R⁴ is H; and
n is 1.

In a preferred embodiment, each variable of Formula (1) comprises the group of corresponding variables in Compounds 1-349. In another preferred aspect, the compound of Formula (1) is selected from the compounds in Examples 1-349.

An aspect of the invention is directed to a pharmaceutical composition comprising the compound of Formula (1) and a pharmaceutically acceptable carrier.

A further aspect is directed to a method for treating a CCR4- or CCR5-mediated disease comprising contacting the compound of Formula (1) or a pharmaceutical composition thereof in a system or a subject, thereby treating said CCR4- or CCR5-mediated disease. Preferably, the system is a cell, tissue or organ, and said subject is human or animal. Preferably, the CCR4- or CCR5-mediated disease is allergic inflammatory conditions, asthma, HIV, an inflammatory demyelinating disease of the central nervous system, an autoimmune disease, multiple sclerosis, experimental autoimmune encephalomyelitis, psoriatic or rheumatoid arthritis, intestinal inflammation, allograft rejection, asthma, cardiovascular disease, atherosclerosis, allergic disease, allergic rhinitis, dermatitis, conjunctivitis, hypersensitivity lung disease, hypersensitivity pneumonitis, eosinophilic pneumonia, delayed-type hypersensitivity, interstitial lung disease (ILD), idiopathic pulmonary fibrosis, ILD associated with rheumatoid arthritis, systemic lupus erythematosus, ankylosing spondylitis, systemic sclerosis, Sjogren's syndrome, polymyositis, dermatomyositis, systemic anaphylaxis, myastenia gravis, juvenile onset diabetes, glomerulonephritis, autoimmune thyroiditis, graft rejection, allograft rejection, graft-versus-host disease, inflammatory bowel disease, Crohn's disease, ulcerative colitis, spondyloarthropathy, scleroderma; psoriasis, inflammatory dermatosis, dermatitis, eczema, acute dermatitis, acute or atopic dermatitis, allergic contact dermatitis, urticaria, vasculitis, eosinphilic myotis, eosiniphilic fasciitis, tumor or cancer. Preferably the disease is a CCR5-mediated disease. More preferably the disease is HIV.

Moreover, the compounds may be supplied as “pro-drugs” or protected forms, which release the compound after administration to a subject. The terms “administration” and or administering” as used herein should be understood to mean providing a compound of the invention to the subject in need of treatment. For example, the compound may carry a protective group which is split off by hydrolysis in body fluids, e.g., in the bloodstream, thus releasing the active compound or is oxidized or reduced in body fluids to release the compound. A discussion of pro-drugs may be found in “Smith and Williams' Introduction to the Principles of Drug Design,” H. J. Smith, Wright, Second Edition, London (1988).

The compounds of the present invention may be administered in the form of pharmaceutically acceptable salts that are non-toxic. The term “pharmaceutically acceptable salt” as used herein means an active ingredient comprising compounds of Formula 1 used in the form of a salt thereof, particularly where the salt form confers on the active ingredient improved pharmacokinetic properties as compared to the free form of the active ingredient or other previously disclosed salt form. The term “pharmaceutically acceptable salt” encompasses all acceptable salts including but not limited to acetate, lactobionate, benzenesulfonate, laurate, benzoate, malate, bicarbonate, maleate, bisulfate, mandelate, bitartarate, mesylate, borate, methylbromide, bromide, methylnitrite, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydradamine, succinate, hydrobromide, tannate, hydrochloride, tartrate, hydroxynaphthoate, teoclate, iodide, tosylate, isothionate, triethiodide, lactate, panoate, valerate, and the like.

Pharmaceutically acceptable salts of the compounds of the present invention can be used as a dosage for modifying solubility or hydrolysis characteristics, or can be used in sustained release or pro-drug formulations. Also, pharmaceutically acceptable salts of the compounds of this invention may include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethylamine, diethylamine, piperazine, tris(hydroxymethyl)aminomethane, and tetramethylammonium hydroxide.

All of the compounds of the invention contain at least one chiral center. The invention includes mixtures of stereoisomers, individual stereoisomers, and enantiomeric mixtures, and mixtures of multiple stereoisomers. In short, the compound may be supplied in any desired degree of chiral purity.

Utility and Administration

In one aspect, the invention is directed to compounds of Formula 1 that may modulate chemokine receptor activity. Chemokine receptors include but are not limited to CCR1, CCR2, CCR3, CCR4, CCR5, CXCR3, and CXCR4.

In one embodiment, the invention provides compounds of Formula 1 that may demonstrate protective effects on target cells from HIV infection by binding specifically to the chemokine receptor, thus affecting the binding of a natural ligand to the CCR5 and/or CXCR4 of a target cell.

In another embodiment, the compounds of the present invention may be useful as agents which affect chemokine receptors, such as CCR1, CCR2, CCR3, CCR4, CCR5, CXCR3, CXCR4 where such chemokine receptors have been correlated as being important mediators of many inflammatory as well as immunoregulatory diseases.

Other diseases that are also implicated with chemokines as mediators include angiogenesis, and tumorigenesis such as brain, and breast tumors. Thus, a compound that modulates the activity of such chemokine receptors is useful for the treatment or prevention of such diseases.

The compounds of Formula 1 described herein may possess biological activity such that they are able to modulate CCR4 or CCR5 chemokine receptor activity and consequent or associated pathogenic processes subsequently mediated by the CCR4 or CCR5 receptor and its natural ligands. In one embodiment, compounds of Formula 1 demonstrate a protective effect against HIV infection by inhibiting the binding of HIV to a chemokine receptor of a target cell such as CCR5 and/or CXCR4. Such modulation is obtained by a method which comprises contacting a target cell with an effective amount of the compound to inhibit the binding of the virus to the chemokine receptor.

Compounds that inhibit chemokine receptor activity and function may be used for the treatment of diseases that are associated with inflammation, including but not limited to, inflammatory or allergic diseases such as asthma, allergic rhinitis, hypersensitivity lung diseases, hypersensitivity pneumonitis, eosinophilic pneumonias, delayed-type hypersensitivity, atherosclerosis, interstitial lung disease (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies, insect sting allergies; autoimmune diseases, such as rheumatoid arthritis, psoriatic arthritis, multiple sclerosis, systemic lupus erythematosus, myastenia gravis, juvenile onset diabetes; glomerulonephritis, autoimmune thyroiditis, graft rejection, including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such as dermatitis, eczema, acute or atopic dermatitis, allergic contact dermatitis, urticaria; vasculitis (e.g., necrotizing, cutaneous, and hypersensitivity vasculitis); eosinphilic myotis, eosiniphilic fasciitis; and cancers.

In addition, compounds that activate or promote chemokine receptor function are used for the treatment of diseases associated with immunosuppression, such as in individuals undergoing chemotherapy, radiation therapy, enhanced wound healing and burn treatment, therapy for autoimmune disease or other drug therapy (e.g., corticosteroid therapy) or combination of conventional drugs used in the treatment of autoimmune diseases and graft/transplantation rejection, which causes immunosuppression; or immunosuppression due to congenital deficiency in receptor function or other causes. Compounds that activate or promote chemokine receptor function are also used for the treatment of infectious diseases, such as parasitic diseases, including but not limited to helminth infections, such as nematodes (round worms); Trichuriasis, Enterobiasis, Ascariasis, Hookworm, Strongyloidiasis, Trichinosis, filariasis; trematodes; visceral worms, visceral larva migtrans (e.g., Toxocara), eosinophilic gastroenteritis (e.g., Anisaki spp., Phocanema ssp.), cutaneous larva migrans (Ancylostona braziliense, Ancylostoma caninum); the malaria-causing protozoan Plasmodium vivax, Human cytomegalovirus, Herpesvirus saimiri, and Kaposi's sarcoma herpesvirus, also known as human herpesvirus 8, and poxvirus Moluscum contagiosum.

Compounds of the present invention may be used in combination with any other active agents or pharmaceutical compositions where such combined therapy is useful to modulate chemokine receptor activity and thereby prevent and treat inflammatory and immunoregulatory diseases.

Furthermore, the compounds may be used in combination with one or more agents useful in the prevention or treatment of HIV. Examples of such agents include:
(1) nucleotide reverse transcriptase inhibitor such as tenofovir disoproxil fumarate; lamivudine/zidovudine; abacavir/lamivudine/zidovudine; emtricitabine; amdoxovir; alovudine; DPC-817; SPD-756; SPD-754; GS7340; ACH-126,443 (beta)-L-F d4C; didanosine, zalcitabine, stavudine, adefovir, adefovir dipivoxil, fozivudine todoxil, etc.;
(2) non-nucleotide reverse transcriptase inhibitor (including an agent having anti-oxidation activity such as immunocal, oltipraz, etc.) such as nevirapine, delavirdine, efavirenz, loviride, immunocal, oltipraz, TMC-125; DPC-083; capravarine; calanolide A; SJ-3366 series, etc.;
(3) protease inhibitors such as saquinavir, lopinavir/ritonavir, atazanavir, fosamprenavir, tipranavir, TMC-114, DPC-684, indinavir, nelfinavir, amprenavir, palinavir, lasinavir, etc.;
(4) entry inhibitors such as T-20; T-1249; PRO-542; PRO-140; TNX-355; BMS-806 series; and 5-Helix;
(5) CCR5-receptor inhibitors such as Sch-C (or SCH351125); Sch-D (or SCH350634); TAK779; UK 427,857 and TAK 449; or CXCR4- receptor inhibitors such as T22, T134, T140, 18 amino acid analogs of polyphemusin II, ALX40-4C, ALK40-4C, AMD3100 and AMD070;
(6) integrase inhibitors such as L-870,810; GW-810781 (S-1360); and
(7) budding inhibitors such as PA-344; and PA-457.

Combinations of compounds of the present invention with HIV agents are not limited to the above examples, but include the combination with any agent useful for the treatment of HIV. Combinations of the compounds of the invention and other HIV agents may be administered separately or in conjunction. The administration of one agent may be prior to, concurrent to, or subsequent to the administration of other agent(s).

The compounds according to the present invention may be administered by oral, intramuscular, intraperitoneal, intravenous, intracisternal injection or infusion, subcutaneous injection, transdermal or transmucosal administration or by implant. They may also be administered by inhalation spray, nasal, vaginal, rectal, sublingual, or topical routes and may be formulated, alone or together, in suitable dosage unit formulations containing conventional non-toxic pharmaceutically acceptable carriers, adjuvants and vehicles appropriate for each route of administration.

The compounds of the invention may be used to treat animals, including mice, rats, horses, cattle, sheep, dogs, cats, and monkeys. However, compounds of the invention can also be used in other species, such as avian species (e.g., chickens). The compounds of the invention may also be effective for use in humans. The term “subject” or alternatively referred to herein as “patient” is intended to be referred to an animal, preferably a mammal, most preferably a human, who has been the object of treatment, observation or experiment. However, the compounds, methods and pharmaceutical compositions of the present invention may be used in the treatment of animals.

The invention also relates to a pharmaceutical composition comprising a pharmaceutically acceptable carrier or diluent and an effective amount of compound of Formula 1. The compounds may be administered alone or as a mixture with a pharmaceutically acceptable carrier (e.g., solid formulations such as tablets, capsules, granules, powders, etc.; liquid formulations such as syrups, injections, etc.). The compounds may be administered orally or non-orally. Examples of hon-oral formulations include injections, drops, suppositories, and pessaryies.

In the treatment or prevention of conditions which require chemokine receptor modulation, an appropriate dosage level will generally be about 0.01 to 500 mg per kg subject body weight per day, and can be administered in singe or multiple doses. Preferably, the dosage level will be about 0.1 to about 250 mg/kg per day. It will be understood that the specific dose level and frequency of dosage for any particular patient may be varied and will depend upon a variety of factors including the activity of the specific compound used, the metabolic stability and length of action of that compound, the age, body weight, general health, sex, diet, mode and time of administration, rate of excretion, drug combination, the severity of the particular condition, and the patient undergoing therapy.

In another aspect of the present invention, a compound of Formula 1 may be used in screening assays for compounds which modulate the activity of chemokine receptors, preferably CCR5 receptors. The ability of a test compound to inhibit gp120 and CD4/CCR5-dependent cell-cell fusion may be measured using a cell fusion assay known in the art.

The compounds of Formula 1 as disclosed herein may be useful for isolating receptor mutants, which can then be made into screening tools for the discovery of even more potent compounds, following procedures described herein and procedures known in the art. The compounds of Formula 1 may also be useful in establishing or characterizing the binding sites of other ligands, including compounds other than those of Formula 1 to chemokine receptors, e.g., by competitive inhibition. The compounds of the present invention may also be useful for the evaluation of putative specific modulators of various chemokine receptors. As appreciated in the art, thorough evaluation of specific agonists and antagonists of the above chemokine receptors has been hampered by the lack of availability of non-peptidyl (metabolically resistant) compounds with high binding affinity for these receptors. Thus, the compounds of this invention are commercial products to be sold for these purposes.

The following examples are offered to illustrate but not to limit the invention.

### EXPERIMENTAL

Compounds of the invention are often readily prepared by known methods; some methods for making compounds and intermediates of the invention are described in a co-pending application by Zhou, et al., which is U.S. patent application Ser. No. 11/453,221.

General Procedures

General Procedure A: Reductive Amination with NaBH(OAc)₃

To a stirred solution of the amine (1 equivalent) in CH₂Cl₂ (concentration ˜0.2M) at room temperature were added the carbonyl compound (1-2 equivalents), glacial AcOH (0-2 equivalents) and sodium triacetoxyborohydride (NaBH(OAc)₃) (˜1.5-3 equivalents) and the resultant solution was stirred at room temperature. In a standard workup, the reaction mixture was poured into either saturated aqueous NaHCO₃ or 1N NaOH. The phases were separated and the aqueous extracted with CH₂Cl₂. The combined organic extracts were dried (Na₂SO₄ or MgSO₄), filtered and concentrated under reduced pressure. The crude material was purified by flash column chromatography on silica gel or by recrystallization.

General Procedure B: Reductive Amination with NaCNBH₃

To a stirred solution of the amine (1 equivalent) in MeOH (concentration ˜0.1M) at room temperature were added the carbonyl compound (1-3 equivalents), glacial AcOH (0-1 equivalents) and sodium cyanoborohydride (NaCNBH₃) (˜1.5-3 equivalents) and the resultant solution was heated to reflux. In a standard workup, the reaction mixture was concentrated under reduced pressure and diluted with saturated aqueous NaHCO₃. The aqueous was extracted with CH₂Cl₂ and the combined organic extracts were dried (Na₂SO₄ or MgSO₄), filtered and concentrated under reduced pressure. The crude material was purified by flash column chromatography on silica gel or by recrystallization.

General Procedure C: BOC Deprotection with TFA

The BOC-protected amine was dissolved in CH₂Cl₂ (˜4 mL/mmol) and trifluoroacetic acid (TFA) (˜2 mL/mmol) was added. The mixture was stirred at room temperature for 0.5-5 hours. In a standard work-up, the mixture was neutralized with saturated aqueous NaHCO₃ or 1N NaOH and the aqueous extracted with CH₂Cl₂. The combined extracts were dried (Na₂SO₄ or MgSO₄), filtered and concentrated under reduced pressure. The crude material was used in the next reaction as is or was purified by flash column chromatography on silica gel.

General Procedure D: Suzuki Coupling with Pd(Ph₃P)₄

To a stirred solution of a halide (1 equivalent), a boronic acid (1.3-3.0 equivalents), sodium carbonate mono-hydrate (1.3-3.0 equivalents) in a mixture of dimethoxyethane/water 4:1 (concentration ˜0.05-0.2M) was added palladium tetrakis- triphenylphosphine (Pd(Ph₃P)₄) (0.1 equivalents). The solution was stirred at 90° C. for ˜18 hours and then treated with water. In a standard work-up, the mixture was extracted with CH₂Cl₂ and the organic layer was dried (Na₂SO₄ or MgSO₄), filtered and concentrated under reduced pressure. The crude material was purified by flash column chromatography or by radial chromatography on silica gel.

General Procedure E: EDCI Coupling

To a stirred solution of a primary or secondary amine (1 equivalent), a carboxylic acid (1.1-2.0 equivalents), 1-hydroxy-benzotriazole hydrate (HOBT) (1.1-2.0 equivalents) and diisopropylethylamine (DIPEA) or N-methylmorpholine (NMM) (1.5-3 equivalents) in CH₂Cl₂ or DMF (concentration ˜0.05-1.5M) was added 1-[3-(dimethylamino)propyl]-3-ethylcarbodiimide hydrochloride (EDCI) (1.1-2.0 equivalents). The solution was stirred at room temperature for 1-3 days and concentrated in vacuo. In a standard work-up, the mixture was diluted with CH₂Cl₂ or EtOAc and washed consecutively with saturated aqueous NaHCO₃ and brine. The organic layer was dried (Na₂SO₄ or MgSO₄), filtered and concentrated under reduced pressure. The crude material was purified by flash column chromatography or by radial chromatography on silica gel.

General Procedure F: IBCF Coupling

To a stirred solution of an acid (1.0 equivalent) in THF (concentration ˜0.2M) at 0° C. was added N-methylmorpholine (NMM) (1.0 equivalent). The solution was stirred for 5 minutes then iso-butylchloroformate (IBCF) (1.0 equivalent) was added. The solution was stirred for 10 minutes before the amine (1.2-2.0 equivalents) was added. The reaction was allowed to return to room temperature and stirred for ˜18 hours. THF was removed under reduced pressure and ethyl acetate was added. The organic layer was washed with saturated aqueous NH₄Cl then with saturated aqueous NaHCO₃. The organic layer was dried (Na₂SO₄ or MgSO₄), filtered and concentrated under reduced pressure. The crude material was purified by flash column chromatography on silica gel or by recrystallization.

General Procedure G: Alkylation

The secondary amine (1.1 equivalents) was dissolved in CH₃CN (concentration ˜0.1M). Diisopropylethylamine (DIPEA) (1.5 equivalents) followed by a halide reagent (1.0 equivalent) were added. The reaction was heated at 50-75° C. for 18 hours. The mixture was concentrated under reduced pressure and CH₂Cl₂ and saturated aqueous NaHCO₃ were added. The aqueous layer was extracted CH₂Cl₂ and the combined organic extracts were dried (Na₂SO₄ or MgSO₄), filtered and concentrated under reduced pressure. The crude material was purified by flash column chromatography on silica gel or by radial chromatography on silica gel.

General Procedure H: Ester Hydrolysis

The ester (1.0 equivalent) was dissolved in a 1:1 MeOH/2N NaOH solution. The reaction was stirred at 50° C. for 5-18 hours. The mixture was concentrated under reduced pressure and distilled water was added. The pH of the solution was adjusted to ˜4-5 with a 6N HCl solution. The aqueous solution was then extracted with CH₂Cl₂ or a mixture of CH₂Cl₂/MeOH (9:1). The combined organic extracts were dried (Na₂SO₄ or MgSO₄), filtered and concentrated under reduced pressure. The crude material was purified by flash column chromatography on silica gel or by recrystallization.

General Procedure I: Cyanide Hydrolysis

The cyanide (1.0 equivalent) was dissolved in a 4:1 to 1:1 EtOH/10N NaOH solution. The reaction was heated to 60-90° C. for 5-18 hours. The mixture was concentrated under reduced pressure and distilled water was added. The pH of the solution was adjusted to ˜4-5 with a 6N HCl solution. The aqueous solution was then extracted with CH₂Cl₂ or a mixture of CH₂Cl₂/MeOH (9:1). The combined organic extracts were dried (Na₂SO₄ or MgSO₄), filtered and concentrated under reduced pressure. The crude material was purified by flash column chromatography on silica gel or by recrystallization.

General Procedure J: Asymmetrical Hydroxyamination (O'Brien, P. et al., J. Chem. Soc., Perkin Trans. (1998) 2519-2526)

To a stirred solution of t-butyl carbamate (2.0 equivalents) in n-propanol (4 L/mol) in a cold-water bath (˜15° C.) was added 1N NaOH (2.05 equivalents), and t-butyl hypochlorite (2.3 equivalents). The mixture was stirred for 10 min and then cooled to 0° C. Solutions of (DHQD)₂PHAL (0.02 equivalents) in n-propanol (4 L/mol), styrene (1 equivalent) in n-propanol (8 L/mol), and K₂OsO₄.2H₂O (0.015 equivalents) were added sequentially. The mixture was stirred at 0° C. until the reaction was complete. The reaction was quenched by the addition of an aqueous Na₂SO₃ solution until any remaining green color was reduced to yellow/brown. The mixture was concentrated under reduced pressure. In a standard workup, the reaction mixture was partitioned between water and CH₂Cl₂. The phases were separated and the aqueous extracted with CH₂Cl₂. The combined organic extracts were dried (Na₂SO₄ or MgSO₄), filtered and concentrated under reduced pressure. The crude material was purified by flash column chromatography on silica gel.

General Procedure K: Cyclization with Triphosgene or Thiophosgene

To a 0° C. solution of the diamine (1 equivalent) and pyridine or triethylamine (Et₃N) (˜2 equivalents) in CH₂Cl₂ (concentration ˜0.05-0.1M) was added triphosgene or thiophosgene (0.35-0.55 equivalents) and the resulting solution was stirred at 0° C. for 5 minutes and at 0° C. or at room temperature for an additional 1-2 hours. In a standard workup, the reaction mixture was poured into saturated aqueous NaHCO₃. The phases were separated and the aqueous extracted with CH₂Cl₂. The combined organic extracts were dried (Na₂SO₄ or MgSO₄), filtered and concentrated under reduced pressure. The crude material was purified by flash column chromatography on silica gel.

General Procedure L: Mitsonobu Reaction and Phthalimide Deprotection

To a solution of N-Boc-2-substituted-glycinol (1 equivalent) in dry THF (3 L/mol) was added phthalimide (1.15 equivalents) and triphenylphosphine (1.2 equivalents). The mixture was cooled in an ice/water bath and DEAD (1.15 equivalents) was added slowly. The mixture was then warmed to room temperature and stirred until the reaction was complete (typically 2-3 hours). The mixture was concentrated under reduced pressure and the resulting residue was suspended in ethanol (5 L/mol) and hydrazine hydrate (10 equivalents) was added. The mixture was stirred for 1-18 hours then diluted with diethyl ether and filtered. The filtrate was concentrated, then diluted with CH₂Cl₂ and the product extracted with 1N HCl in the aqueous. The resulting acidic aqueous solution was basified with 1N or 10 N NaOH to pH˜11-12 and then extracted with CH₂Cl₂. The organic layers were combined, dried (Na₂SO₄ or MgSO₄), filtered and concentrated under reduced pressure. The crude material was purified by flash column chromatography on silica gel.

### Intermediates

### (R)-1-Cyclohexyl-4-phenyl-3-piperidin-4-yl-imidazolidin-2-one

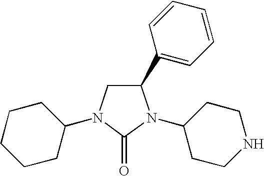

Di-t-butyl dicarbonate (31.82 g, 146.8 mmol) was added to a cooled (0° C.) solution of (R)-(−)-2-phenylglycinol (20.00 g, 145.8 mmol) and triethylamine (24.4 mL, 175.0 mmol) in dry tetrahydrofuran (292 mL). The mixture was stirred for 3 h at 0° C. then concentrated in vacuo. The residue was taken up in CH₂Cl₂ (200 mL) and washed with 1N HCl (240 mL). The aqueous layer was extracted with CH₂Cl₂ (2×100 mL). The combined organic layers were dried (Na₂SO₄) and concentrated in vacuo to afford ((R)-2-hydroxy-1-phenyl-ethyl)-carbamic acid tert-butyl ester (34.59 g, >99%) as a white solid. ¹H NMR (CDCl₃) δ 1.43 (s, 9H), 2.29 (br s, 1H), 3.84-3.87 (m, 2H), 4.79 (br s, 1H), 7.29-7.39 (m, 5H).

Following general procedure L: to a cooled (0° C.) solution of ((R)-2-hydroxy-1-phenyl-ethyl)-carbamic acid tert-butyl ester (34.59 g, 145.8 mmol), phthalimide (22.5 g, 153.1 mmol) and triphenylphosphine (42.1 g, 160.4 mmol) in dry tetrahydrofuran (1 L) was added diethyl azodicarboxylate (24 mL, 153.1 mmol), slowly over 10 min. Stirred for an additional 10 min. then warmed to ambient temperature. Stirred for an additional 5 h, then concentrated to a white solid residue. The residue was taken up in ethanol (1 L) to which was added hydrazine hydrate (50 mL, 1.61 mol) and warmed to reflux for 1 h. Standard work-up and purification by column chromatography on silica gel (CH₂Cl_{2/}MeOH/NH₄OH, 93:5:2) afforded ((R)-2-amino-1-phenyl-ethyl)-carbamic acid tert-butyl ester (27.27 g. 79%) as a white solid. ¹H NMR (CDCl₃) δ 1.09 (br s, 2H), 1.42 (s, 9H), 2.99-3.01 (m, 2H), 4.65 (br s, 1H), 7.28-7.34 (m, 5H).

Using general procedure A followed by general procedure C, ((R)-2-amino-1-phenyl-ethyl)-carbamic acid tert-butyl ester (8.00 g. 33.85 mmol), cyclohexanone (3.32 mL, 33.85 mmol) and NaBH(OAc)₃ (8.61 g, 40.62 mmol) in CH₂Cl₂ (340 mL) afford (R)-N²-cyclohexyl-1-phenyl-ethane-1,2-diamine (7.67 g, >99%) as a yellow oil. The yellow oil was carried on without further purification.

Following general procedure A: the above diamine (7.67 g, 33.85 mmol), 1-boc-4-piperidone (6.74 g, 33.85 mmol) and NaBH(OAc)₃ (8.61 g, 40.62 mmol) in CH₂Cl₂ (335 mL) afforded 4-((R)-2-cyclohexylamino-1-phenyl-ethylamino)-piperidine-1-carboxylic acid tert-butyl ester (11.59 g, 85%) as a light yellow oil. ¹H NMR (CDCl₃) δ 0.97-1.17 (m, 2H), 1.14-1.31 (m, 5H), 1.43 (s, 9H), 1.64-1.75 (m, 3H), 1.78-1.92 (m, 3H), 2.33-2.52 (m, 2H), 2.60-2.74 (m, 3H), 2.81 (dd, 1H, J=10.0, 4.2 Hz), 3.84 (dd, 1H, J=10.0, 4.2 Hz), 3.88-4.00 (m, 2H), 7.26-7.36 (m, 5H).

Following general procedure K: to a cooled (0° C.) solution of 4-((R)-2-cyclohexylamino-1-phenyl-ethylamino)-piperidine-1-carboxylic acid tert-butyl ester (11.59 g, 28.85 mmol) and pyridine (9.3 mL, 115.4 mmol) in CH₂Cl₂ was slowly added triphosgene (8.56 g, 28.85 mmol). The ice bath was removed and the mixture was stirred at ambient temperature for 14 h. Standard work-up afforded the crude carbamate as a brown oil. Following general procedure C, the carbamate afforded (R)-1-cyclohexyl-4-phenyl-3-piperidin-4-yl-imidazolidin-2-one as a tacky orange foamy solid (8.4 g, 89%). ¹H NMR (CDCl₃) δ 0.95-1.15 (m, 2H), 1.19-1.49 (m, 5H), 1.65-1.81 (m, 6H), 2.48 (dt, 1H, J=12.0, 3.1 Hz), 2.60 (dt, 1H, J=12.0, 3.1 Hz), 2.85-2.92 (m, 1H), 3.03-3.10 (m, 2H), 3.63 (t, 1H, J=8.9 Hz), 3.68-3.82 (m, 3H), 4.58 (dd, 1H, J=9.6, 7.0 Hz), 8.28-7.33 (m, 5H).

### (R)-4-Phenyl-3-piperidin-4-yl-1-(tetrahydro-pyran-4-yl)-imidazolidin-2-one

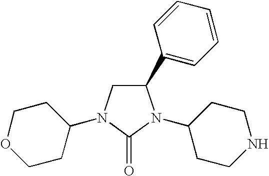

(R)-4-Phenyl-3-piperidin-4-yl-1-(tetrahydro-pyran-4-yl)-imidazolidin-2-one was prepared using the same chemistry as (R)-1-cyclohexyl-4-phenyl-3-piperidin-4-yl-imidazolidin-2-one except that tetrahydro-4H-pyran-4-one was used in lieu of cyclohexanone. ¹H NMR (CDCl₃) δ 1.07 (qd, 1H, J=12.4, 4.3 Hz), 1.41-1.50 (m, 2H), 1.56-1.84 (m, 6H), 2.47 (td, 1H, J=12.3, 2.5 Hz), 2.58 (td, 1H, J=12.0, 3.1 Hz), 2.84-2.92 (m, 1H), 3.02-3.09 (m, 1H), 3.06 (dd, 1H, J=8.4, 6.9 Hz), 3.43-3.52 (m, 2H), 3.64 (t, 1H, J=8.9 Hz), 3.69 (tt, 1H, J=11.9, 3.9 Hz), 3.94-4.10 (m, 3H), 4.60 (dd, 1H, J=9.5, 6.7 Hz), 7.28-7.37 (m, 5H).

### (R)-1-(8-Oxa-bicyclo[3.2.1]oct-3-yl)-4-phenyl-3-piperidin-4-yl-imidazolidin-2-one

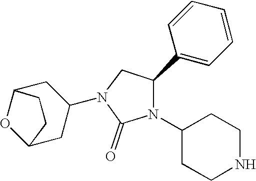

(R)-1-(8-Oxa-bicyclo[3.2.1]oct-3-yl)-4-phenyl-3-piperidin-4-yl-imidazolidin-2-one was prepared using the same chemistry as (R)-1-cyclohexyl-4-phenyl-3-piperidin-4-yl-imidazolidin-2-one except that 8-oxa-bicyclo[3.2.1]octan-3-one was used in lieu of cyclohexanone. ¹H NMR (CDCl₃) δ 1.07-1.15 (m, 1H), 1.44-1.57 (m, 4H), 1.63-1.73 (m, 4H), 1.92-1.97 (m, 2H), 2.21-2.30 (m, 2H), 2.43-2.63 (m, 2H), 2.88 (d, 1H, J=12.8 Hz), 3.00-3.08 (m, 2H), 3.59 (t, 1H, J=9.0 Hz), 3.73 (m, 1H), 4.02 (m, 1H), 4.42 (m, 2H), 4.57 (dd, 1H, J=9.0, 6.0 Hz), 7.29-7.35 (m, 5H).

### (R)-4-Phenyl-3-piperidin-4-yl-oxazolidin-2-one

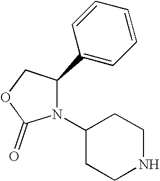

Following general procedure K: to a cooled (0° C.) solution of (R)-4-(2-hydroxy-1-phenyl-ethylamino)-piperidine-1-carboxylic acid tert-butyl ester (0.30 g, 0.93 mmol) and pyridine (0.11 mL, 1.4 mmol) in dry dichloromethane (3 mL) was slowly added triphosgene (138 mg, 0.47 mmol). The ice bath was removed and the mixture was gradually warmed to ambient temperature over 1 h. Standard work-up afforded (R)-4-(2-oxo-4-phenyl-oxazolidin-3-yl)-piperidine-1-carboxylic acid tert-butyl ester as a yellow solid (0.33 g). Following general procedure C, the crude product afforded (R)-4-phenyl-3-piperidin-4-yl-oxazolidin-2-one as a pale yellow solid (208 mg, 91% over 2 steps). ¹H NMR (CDCl₃) δ 1.14 (dq, 1H, J=7.5, 3.6 Hz), 1.73 (d, 1H, J=10.5 Hz), 1.82 (m, 2H), 2.48 (dt, 1H, J=12.0, 3.6 Hz), 2.57 (dt, 1H, J=12.0, 3.6 Hz), 2.92 (d, 1H, J=10.8 Hz), 3.09 (d, 1H, J=10.8 Hz), 3.67 (m, 1H), 4.09 (m, 1H), 4.58 (t, 1H, J=9.0 Hz), 4.81 (m, 1H), 7.37 (m, 5H).

### (R)-4-(3-Chloro-phenyl)-3-piperidin-4-yl-1-(tetrahydro-pyran-4-yl)-imidazolidin-2-one

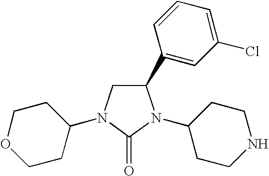

Using general procedure J, 3-chlorostyrene (28.0 g, 200 mmol) was converted to [(R)-1-(3-chloro-phenyl)-2-hydroxy-ethyl]-carbamic acid tert-butyl ester (27.9 g, 51% yield, 99% purity by HPLC, 93.7% ee by HPLC). ¹H NMR (CD₃OD) δ 3.63-3.72 (m, 2H), 4.63-4.67 (m, 1H), 7.26-7.37 (m, 2H), 7.43 (s, 1H).

Using general procedure L, [(R)-1-(3-chloro-phenyl)-2-hydroxy-ethyl]-carbamic acid tert-butyl ester (33.81 g, 122 mmol) was converted to [(R)-2-amino-1-(3-chloro-phenyl)-ethyl]-carbamic acid tert-butyl ester (27.73 g, 82%). ¹H NMR (CDCl₃) δ 1.42 (s, 9H), 3.98-3.99 (m, 2H), 4.57-4.70 (m, 1H), 5.38-5.45 (m, 1H), 7.17-7.18 (m, 1H), 7.23-7.28 (m, 3H).

(R)-4-(3-Chloro-phenyl)-3-piperidin-4-yl-1-(tetrahydro-pyran-4-yl)-imidazolidin-2-one (30.0 g, 88.7% over 5 steps) was prepared using the same chemistry as that for (R)-1-cyclohexyl-4-phenyl-3-piperidin-4-yl-imidazolidin-2-one except that [(R)-2-amino-1-(3-chloro-phenyl)-ethyl]-carbamic acid tert-butyl ester (26.5 g, 97.9 mmol) was used in lieu of ((R)-2-amino-1-phenyl-ethyl)-carbamic acid tert-butyl ester and tetrahydro-4H-pyranone was used in lieu of cyclohexanone. ¹H NMR (CDCl₃) δ 1.05-1.19 (m, 1H), 1.46-1.53 (m, 1H), 1.60-1.89 (m, 9H), 2.53 (dt, 1H, J=12.4, 2.2 Hz), 2.63 (dt, 1H, J=11.8, 3.7 Hz), 2.91-2.99 (m, 1H), 3.04 (dd, 1H, J=8.8, 6.6 Hz), 3.08-3.15 (m, 1H), 3.44-3.57 (m, 2H), 3.63-3.80 (m, 2H), 3.97-4.10 (m, 3H), 4.58 (dd, 1H, J=9.2, 6.6 Hz), 7.19-7.23 (m, 1H), 7.28-7.33 (m, 3H).

### (R)-4-(3-Chloro-phenyl)-3-piperidin-4-yl-oxazolidin-2-one

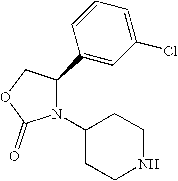

Following general procedure C, [(R)-1-(3-chloro-phenyl)-2-hydroxy-ethyl]-carbamic acid tert-butyl ester (446 mg, 1.77 mmol) afforded (R)-2-amino-(3-chloro-phenyl)-ethanol as a brown oil (153 mg, 57%). ¹H NMR (CDCl₃) δ 1.79 (s, 2H), 3.53 (t, 2H, J=9.0 Hz), 3.74 (dd, 1H, J=10.5, 3.3 Hz), 4.04 (m, 1H), 7.18-7.29 (m, 3H), 7.34 (s, 1H).

Using general procedure A, the above amine (153 mg, 1.0 mmol) and N-Boc-4-piperidone (209 mg, 1.05 mmol) gave 4-[(R)-1-(3-chloro-phenyl)-2-hydroxy-ethylamino]-piperidine-1-carboxylic acid tert-butyl ester as a brown oil (0.38 g). Following general procedure K: to a cooled (0° C.) solution of the above alcohol (0.38 g, 1.0 mmol) and pyridine (0.12 mL, 1.5 mmol) in CH₂Cl₂ (5 mL) was slowly added triphosgene (148 mg, 0.50 mmol). The ice bath was removed and the mixture was gradually warmed to ambient temperature over 1 h. Standard work-up afforded 4-[(R)-4-(3-chloro-phenyl)-2-oxo-oxazolidin-3-yl]-piperidine-1-carboxylic acid tert-butyl ester as a yellow solid (0.40 g). Following general procedure C, the crude product afforded (R)-4-(3-chloro-phenyl)-3-piperidin-4-yl-oxazolidin-2-one as a pale yellow solid (209 mg, 75%, 3 steps). ¹H NMR (CDCl₃) δ 1.16 (dq, 1H, J=7.5, 3.6 Hz), 1.55 (d, 1H, J=10.5 Hz), 1.70 (m, 2H), 2.52 (t, 1H, J=11.1 Hz), 2.65 (m, 1H), 2.96 (d, 1H, J=10.8 Hz), 3.12 (d, 1H, J=10.8 Hz), 3.70 (m, 1H), 4.05 (m, 1H), 4.58 (t, 1H, J=9.0 Hz), 4.78 (m, 1H), 7.22 (s, 1H), 7.30 (m, 3H).

### (R)-4-(3-Chloro-phenyl)-1-cyclopentyl-3-piperidin-4-yl-imidazolidin-2-one

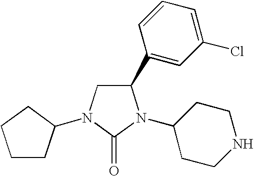

(R)-4-(3-Chloro-phenyl)-1-cyclopentyl-3-piperidin-4-yl-imidazolidin-2-one (320 mg, 56% over 5 steps) was prepared using the same chemistry as that for (R)-1-cyclohexyl-4-phenyl-3-piperidin-4-yl-imidazolidin-2-one except that [(R)-2-amino-1-(3-chloro-phenyl)-ethyl]-carbamic acid tert-butyl ester (450 mg, 1.66 mmol) was used in lieu of (2-amino-1-phenyl-ethyl)-carbamic acid tert-butyl ester and cyclopentanone was used in lieu of cyclohexanone. ¹H NMR (CDCl₃) δ 1.01-1.15 (m, 1H), 1.41-1.88 (m, 11H), 2.46-2.66 (m, 2H), 2.89-2.94 (m, 1H), 2.89-3.10 (m, 2H), 3.64 (t, 1H, J=9.0 Hz), 3.69-3.79 (m, 1H), 4.30-4.39 (m, 1H), 4.56 (dd, 1H, J=9.0, 6.6 Hz), 7.21-7.29 (m, 3H), 7.34 (s, 1H).

### (R)-4-(3-Fluoro-phenyl)-3-piperidin-4-yl-1-(tetrahydro-pyran-4-yl)-imidazolidin-2-one

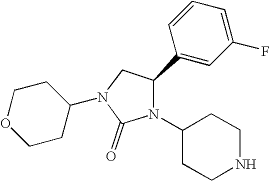

Following general procedure J, 3-fluorostyrene (4.98 g, 40.8 mmol) was converted to [(R)-1-(3-fluoro-phenyl)-2-hydroxy-ethyl]-carbamic acid tert-butyl ester (5.283 g, 20.7 mmol, 51% yield, 91% EE). ¹H NMR (CDCl₃) δ 1.43 (s, 9H), 2.04 (br s, 1H), 3.85 (m, 2H), 4.77 (br s, 1H), 5.24 (br s, 1H), 6.95-7.04 (m, 2H), 7.08 (d, 1H, J=7.5 Hz), 7.32 (m, 1H).

Following general procedure L, the above alcohol (3.00 g, 11.8 mmol) afforded [(R)-2-amino-1-(3-fluoro-phenyl)-ethyl]-carbamic acid tert-butyl ester as a brown solid (2.156 g, 72% over 2 steps). ¹H NMR (CDCl₃) δ 1.43 (s, 9H), 3.01 (s, 2H), 4.66 (s, 1H), 5.42 (s, 1H), 6.92-7.01 (m, 2H), 7.06 (d, 1H, J=7.5 Hz), 7.30 (m, 1H).

(R)-4-(3-Fluoro-phenyl)-3-piperidin-4-yl-1-(tetrahydro-pyran-4-yl)-imidazolidin-2-one (1.138 g, 38% over 5 steps) was prepared using the same chemistry as that for (R)-1-cyclohexyl-4-phenyl-3-piperidin-4-yl-imidazolidin-2-one except that [(R)-2-amino-1-(3-fluoro-phenyl)-ethyl]-carbamic acid tert-butyl ester (2.156 g, 8.5 mmol) was used in lieu of ((R)-2-amino-1-phenyl-ethyl)-carbamic acid tert-butyl ester and tetrahydro-4H-pyranone was used in lieu of cyclohexanone. ¹H NMR (CDCl₃) δ 1.23 (dq, 1H, J=7.5, 3.6 Hz), 1.45 (d, 1H, J=10.8 Hz), 1.64-1.76 (m, 5H), 1.82 (dq, 1H, J=7.5, 2.5 Hz), 2.58 (dt, 1H, J=7.5, 2.5 Hz), 2.68 (dt, 1H, J=7.5, 2.5 Hz), 2.95 (d, 1H, J=10.8 Hz), 3.06 (m, 1H), 3.17 (d, 1H, J=10.8 Hz), 3.30 (m, 1H), 3.48 (m, 2H), 3.64 (t, 1H, J=9.0 Hz), 3.75 (m, 1H), 4.00 (m, 3H), 4.60 (m, 1H), 7.00 (m, 2H), 7.10 (d, 1H, J=6.9 Hz), 7.30 (m, 1H).

### (R)-3-Piperidin-4-yl-1-(tetrahydro-pyran-4-yl)-4-m-tolyl-imidazolidin-2-one

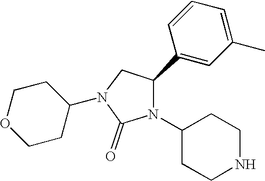

Following general procedure J, 3-methylstyrene (3.00 g, 25.3 mmol) was converted to ((R)-2-hydroxy-1-m-tolyl-ethyl)-carbamic acid tert-butyl ester (3.20 g, 12.7 mmol, 50% yield, 93% EE). ¹H NMR (CDCl₃) δ 1.43 (s, 9H), 2.35 (s, 3H), 2.36 (s, 1H), 3.83 (m, 2H), 4.74 (s, 1H), 5.20 (s, 1H), 7.11 (s, 3H), 7.23 (m, 1H).

Following general procedure L, the above alcohol (2.50 g, 9.9 mmol) afforded ((R)-2-amino-1-m-tolyl-ethyl)-carbamic acid tert-butyl ester as a brown solid (1.40 g, 57% over 2 steps). ¹H NMR (CDCl₃) δ 1.42 (s, 9H), 2.35 (s, 3H), 2.99 (m, 2H), 4.60 (s, 1H), 5.22 (s, 1H), 7.08 (s, 3H), 7.22 (d, 1H, J=7.5 Hz).

(R)-3-Piperidin-4-yl-1-(tetrahydro-pyran-4-yl)-4-m-tolyl-imidazolidin-2-one (0.31 g, 16% over 5 steps) was prepared using the same chemistry as that for the (R)-1-cyclohexyl-4-phenyl-3-piperidin-4-yl-imidazolidin-2-one except that ((R)-2-amino-1-m-tolyl-ethyl)-carbamic acid tert-butyl ester (1.40 g, 5.6 mmol) was used in lieu of ((R)-2-amino-1-phenyl-ethyl)-carbamic acid tert-butyl ester and tetrahydro-4H-pyranone was used in lieu of cyclohexanone. ¹H NMR (CDCl₃) δ 1.23 (dq, 1H, J=7.5, 3.6 Hz), 1.45 (d, 1H, J=12.0 Hz), 1.60-1.75 (m, 5H), 1.82 (dq, 1H, J=7.5, 2.5 Hz), 2.35 (s, 3H), 2.57 (dt, 1H, J=7.5, 2.5 Hz), 2.68 (dt, 1H, J=7.5, 2.5 Hz), 2.95 (d, 1H, J=12.3 Hz), 3.49 (m, 2H), 3.64 (t, 1H, J=9.0 Hz), 3.75 (m, 1H), 4.00 (m, 3H), 4.56 (m, 1H), 7.12 (m, 3H), 7.22 (d, 1H, J=6.9 Hz).

### 4-(2-Fluoro-5-methyl-phenyl)-3-piperidin-4-yl-1-(tetrahydro-pyran-4-yl)-imidazolidin-2-one

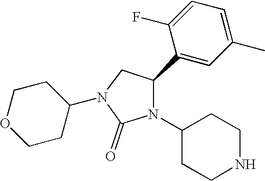

To a solution of NaCN (0.99 g, 19.7 mmol), NH₄Cl (1.06 g, 19.7 mmol) in ammonium hydroxide (28%, 10 mL) was added a solution of 3-fluoro-5-methylbenzaldehyde (1.36 g, 9.85 mmol) in methanol (5 mL). The mixture was stirred at rt for 2 h. The solvents were removed by vacuum evaporation. To the residue was added 6 N HCl (8 mL) and the mixture was heated at reflux for 90 min. The solvents were removed by vacuum evaporation. To the residue was added dry THF (25 mL) followed by BH₃-THF complex (1.0 M in THF, 30 mL, 30.0 mmol). The mixture was heated at reflux for 2 h. After cooling, methanol (8 mL) was added and the mixture was refluxed for another 15 min. The solvents were evaporated under reduced pressure. The residue was diluted with saturated aqueous NaHCO₃ (30 mL), extracted with 5:1 CHCl₃-i-PrOH (3×200 mL). The extracts were dried over Na₂SO₄ and the solvents were evaporated to give the crude product. This was purified by silica gel column chromatography (95:4:1, CH₂Cl₂/MeOH/NH₄OH) to give 2-amino-2-(2-fluoro-5-methyl-phenyl)-ethanol (984 mg, 59%).

To a solution of 2-amino-2-(2-fluoro-5-methyl-phenyl)-ethanol (984 mg, 5.82 mmol) in THF (15 mL) and triethylamine (1.22 mL, 8.73 mmol) was added Boc₂O (1.33 g, 6.10 mmol). The mixture was stirred at rt for 2 h. The solvents were removed by vacuum evaporation. The residue was diluted with water (30 mL), acidified with 1 N HCl to pH=4˜5, and extracted with CH₂Cl₂ (3×20 mL). The extracts were dried over Na₂SO₄ and the solvents were evaporated to give [1-(2-fluoro-5-methyl-phenyl)-2-hydroxy-ethyl]-carbamic acid tert-butyl ester (1.57 g, 100%).

To a solution of above product (1.57 g, 5.87 mmol), phthalimide (0.949 g, 6.45 mmol), Ph₃P (1.844 g, 7.04 mmol) in dry THF (30 mL) at 0° C. was added DEAD (1.01 mL, 6.45 mmol) dropwise. The mixture was then stirred at rt for 2 h. The solvent was removed by evaporation under reduced pressure to afford the crude product.

To a mixture of above crude product in ethanol (35 mL) was added hydrazine hydrate (7 mL). The mixture was stirred at rt for 16 h. Standard work-up and purification by silica gel column chromatography (95:4:1, CH₂Cl₂/MeOH/NH₄OH) gave [2-amino-1-(2-fluoro-5-methyl-phenyl)-ethyl]-carbamic acid tert-butyl ester as a white solid (1.40 g, 89% over 2 steps).

Following general procedure A, a solution of above product (406 mg, 1.51 mmol), tetrahydro-pyran-4-one (159 mg, 1.59 mmol), and NaBH(OAc)₃ (473 mg, 2.12 mmol) in CH₂Cl₂ (5 mL) was stirred at room temperature for 17 h to give crude [1-(2-fluoro-5-methyl-phenyl)-2-(tetrahydro-pyran-4-ylamino)-ethyl]-carbamic acid tert-butyl ester (533 mg, 100%).

Following general procedure C, the above product (533 mg, 1.51 mmol) was treated with TFA (1.5 mL) in CH₂Cl₂ (5 mL) for 2 h to give crude 1-(2-fluoro-5-methyl-phenyl)-N²-(tetrahydro-pyran-4-yl)-ethane-1,2-diamine (382 mg, 100%).

Following general procedure A, a solution of above product (382 mg, 1.51 mmol), 1-Boc-4-piperidone (317 mg, 1.59 mmol) and NaBH(OAc)₃ (473 mg, 2.12 mmol) in CH₂Cl₂ (5 mL) was stirred at room temperature for 17 h to give, after purifying by column chromatography, 4-[1-(2-fluoro-5-methyl-phenyl)-2-(tetrahydro-pyran-4-ylamino)-ethylamino]-piperidine-1-carboxylic acid tert-butyl ester (587 mg, 89%).

Following general procedure K, to a solution of above product (673 mg, 1.55 mmol) and pyridine (251 μL, 3.10 mmol) in dry CH₂Cl₂ (7.5 mL) at 0° C. was added triphosgene (1844 mg, 0.62 mmol) in portions. After stirring at 0° C. for 30 min, the mixture was stirred at rt for another 2 h to give 4-[5-(2-fluoro-5-methyl-phenyl)-2-oxo-3-(tetrahydro-pyran-4-yl)-imidazolidin-1-yl]-piperidine-1-carboxylic acid tert-butyl ester (519 mg, 73%).

Following general procedure C, the above product (519 mg, 1.13 mmol) was treated with TFA (1.7 mL) in CH₂Cl₂ (5.1 mL) for 2 h to give 4-(2-fluoro-5-methyl-phenyl)-3-piperidin-4-yl-1-(tetrahydro-pyran-4-yl)-imidazolidin-2-one (406 mg, 100%). ¹H NMR (CDCl₃) δ 1.04-1.18 (m, 1H), 1.49-1.78 (m, 7H), 1.84 (s, 1H), 2.29 (s, 3H), 2.47-2.65 (m, 2H), 2.92 (d, 1H, J=12.6 Hz), 3.04-3.10 (m, 2H), 3.43-3.52 (m, 2H), 3.64-3.75 (m, 2H), 3.96-4.08 (m, 3H), 4.94 (dd, 1H, J=9.6, 6.3 Hz), 6.91 (t, 1H, J=9.3 Hz), 7.03-7.07 (m, 1H), 7.16 (dd, 1H, J=6.9, 1.5 Hz).

### (R)-4-(3-Methyl-phenyl)-3-piperidin-4-yl-oxazolidin-2-one

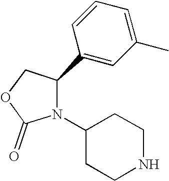

Following general procedure C, ((R)-2-hydroxy-1-m-tolyl-ethyl)-carbamic acid tert-butyl ester (412 mg, 1.78 mmol) afforded (R)-2-amino-(3-methyl-phenyl)-ethanol as a brown oil (153 mg, 66%). ¹H NMR (CDCl₃) δ 1.85 (s, 2H), 2.36 (s, 3H), 3.54 (t, 2H, J=9.0 Hz), 3.74 (d, 1H, J=9.3 Hz), 4.00 (m, 1H), 7.11 (m, 3H), 7.26 (m, 1H).

Using general procedure A, the above amine (153 mg, 1.2 mmol) and N-Boc-4-piperidone (244 mg, 1.22 mmol) gave 4-[(R)-1-(3-methyl-phenyl)-2-hydroxy-ethylamino]-piperidine-1-carboxylic acid tert-butyl ester as a brown oil (0.38 g).

Following general procedure K: to a cooled (0° C.) solution of the above alcohol (0.38 g, 1.2 mmol) and pyridine (0.14 mL, 1.8 mmol) in dry dichloromethane (6 mL) was slowly added triphosgene (174 mg, 0.59 mmol). The ice bath was removed and the mixture was gradually warmed to ambient temperature over 1 h. Standard work-up afforded 4-[(R)-4-(3-methyl-phenyl)-2-oxo-oxazolidin-3-yl]-piperidine-1-carboxylic acid tert-butyl ester as a yellow solid (0.39 g). Following general procedure C, the crude product afforded (R)-4-(3-methyl-phenyl)-3-piperidin-4-yl-oxazolidin-2-one as a pale yellow solid (254 mg, 90% over 3 steps). ¹H NMR (CDCl₃) δ 1.16 (dq, 1H, J=7.5, 3.6 Hz), 1.55 (m, 1H), 1.79 (m, 1H), 1.80 (dq, 1H, J=7.5, 3.6 Hz), 2.36 (s, 3H), 2.48 (tt, 1H, J=12.6, 3.6 Hz), 2.60 (tt, 1H, J=12.3, 3.3 Hz), 2.91 (d, 1H, J=10.2 Hz), 3.10 (d, 1H, J=10.8 Hz), 3.66 (m, 1H), 4.07 (m, 1H), 4.58 (t, 1H, J=9.0 Hz), 4.78 (m, 1H), 7.13 (m, 3H), 7.27 (m, 1H).

### (R)-4-Phenyl-3-piperidin-4-yl-oxazolidine-2-thione

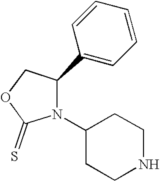

Using general procedure A, (R)-phenylglycinol (1.35 g, 9.84 mmol) and 1-Boc-4-piperidone (2.0 g, 10.0 mmol) afforded crude 4-((R)-2-hydroxy-1-phenylethylamino)-piperidine-1-carboxylic acid tert-butyl ester (3.2 g).

To a solution of the above amino-alcohol (9.84 mmol) in DMF (20 mL) was added 1,1-thiocarbonyldiimidazole (1.84 g, 10.3 mmol). The reaction mixture was stirred at room temperature for 18 h. Aqueous work-up afforded crude 4-((R)-4-phenyl-2-thioxooxazolidin-3-yl)-piperidine-1-carboxylic acid tert-butyl ester (3.6 g, quant).

Using general procedure C, the above thiooxazolidinone (208 mg, 0.57 mmol) afforded (R)-4-phenyl-3-piperidin-4-yl-oxazolidine-2-thione (204 mg) as crude material. Note: the crude material was contaminated with 15% molar of (R)-4-phenyl-3-piperidin-4-yl-oxazolidin-2-one. Purification of the crude material (102 mg) by chromatography on silica gel (5% MeOH in CH₂Cl₂, 2% NH₄OH) afforded pure desired product (35 mg, 34% over 3 steps). ¹H NMR (CDCl₃) δ 0.93 (qd, 1H, J=12.3, 4.4 Hz), 1.58-1.69 (m, 1H), 1.78 (qd, 1H, J=12.0, 4.4 Hz), 1.87-1.98 (m, 1H), 2.40-2.53 (m, 1H), 2.56 (td, 1H, J=12.3, 2.7 Hz), 2.72 (td, 1H, J=12.3, 2.7 Hz), 2.86-2.97 (m, 1H), 3.11-3.21 (m, 1H), 4.35 (dd, 1H, J=8.6, 3.9 Hz), 4.47 (tt, 1H, J=12.3, 4.0 Hz), 4.74 (t, 1H, J=8.6 Hz), 4.97 (dd, 1H, J=9.2, 4.0 Hz), 7.19-7.42 (m, 5H).

### (R)-3-Piperidin-4-yl-1-(tetrahydro-pyran-4-yl)-4-thiophen-3-yl-imidazolidin-2-one

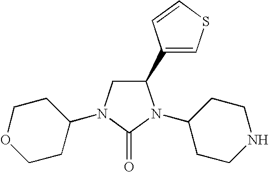

Using general procedure J, 3-vinylthiophene (Nicolas, M., et al., J. Heterocycl. Chem. (1999) 36:1105-1106) (1.30 g, 11.8 mmol) was converted to ((R)-2-hydroxy-1-thiophen-3-yl-ethyl)-carbamic acid tert-butyl ester (1.58 g, 51% yield, 92% ee by ¹H NMR (mosher ester)). ¹H NMR (CD₃OD) δ 3.67-3.80 (m, 2H), 4.76-4.81 (m, 1H), 7.10 (dd, 1H, J=5.0, 1.4 Hz), 7.26-7.28 (m, 1H), 7.39 (dd, 1H, J=5.3, 3.1 Hz).

Using general procedure L, ((R)-2-hydroxy-1-thiophen-3-yl-ethyl)-carbamic acid tert-butyl ester (1.58 g, 6.5 mmol) was converted to ((R)-2-amino-1-thiophen-3-yl-ethyl)-carbamic acid tert-butyl ester (1.46 g, 93%). ¹H NMR (CDCl₃) δ 2.83-2.95 (m, 2H), 4.68-4.77 (m, 1H), 7.09 (dd, 1H, J=5.1, 1.5 Hz), 7.24-7.26 (m, 1H), 7.42 (dd, 1H, J=5.10, 2.8 Hz).

(R)-3-Piperidin-4-yl-1-(tetrahydro-pyran-4-yl)-4-thiophen-3-yl-imidazolidin-2-one (1.65 g, 82% over 5 steps) was prepared using the same chemistry as that for (R)-1-cyclohexyl-4-phenyl-3-piperidin-4-yl-imidazolidin-2-one except that ((R)-2-amino-1-thiophen-3-yl-ethyl)-carbamic acid tert-butyl ester (1.46 g, 6.0 mmol) was used in lieu of ((R)-2-amino-1-phenyl-ethyl)-carbamic acid tert-butyl ester and tetrahydro-4H-pyranone was used in lieu of cyclohexanone. ¹H NMR (CDCl₃) δ 1.10 (dq, 1H, J=12.3, 4.0 Hz), 1.44-1.54 (m, 3H), 1.62-1.72 (m, 5H), 1.79 (dq, 1H, J=12.3, 4.4 Hz), 2.51 (dt, 1H, J=12.3, 2.6 Hz), 2.60 (dt, 1H, J=12.3, 2.8 Hz), 2.88-2.96 (m, 1H), 3.04-3.13 (m, 2H), 5.41-5.53 (m, 2H), 3.59 (t, 1H, J=8.8 Hz), 3.69 (tt, 1H, J=12.0, 3.9 Hz), 3.97-4.10 (m, 3H), 4.75 (dd, 1H, J=9.2, 6.6 Hz), 7.07 (d, 1H, J=5.3 Hz), 7.20 (d, 1H, J=3.1 Hz), 7.32 (dd, 1H, J=5.1, 2.8 Hz).

### (R)-1-Cyclopentyl-3-piperidin-4-yl-4-thiophen-3-yl-imidazolidin-2-one

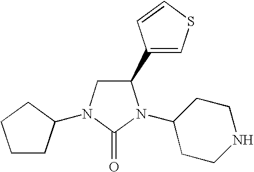

(R)-1-Cyclopentyl-3-piperidin-4-yl-4-thiophen-3-yl-imidazolidin-2-one (70 mg, 23% over 5 steps) was prepared using the same chemistry as that for (R)-1-cyclohexyl-4-phenyl-3-piperidin-4-yl-imidazolidin-2-one except that ((R)-2-amino-1-thiophen-3-yl-ethyl)-carbamic acid tert-butyl ester (1.46 g, 6.0 mmol) was used in lieu of ((R)-2-amino-1-phenyl-ethyl)-carbamic acid tert-butyl ester and cyclopentanone was used in lieu of cyclohexanone. ¹H NMR (CDCl₃) δ 1.03-1.17 (dq, 1H, J=12.6, 4.5 Hz), 1.25 (s, 2H), 1.43-1.67 (m, 7H), 1.73-1.86 (m, 3H), 2.46-2.65 (m, 2H), 2.90-2.94 (d, 1H, J=12 Hz), 3.02-3.15 (dd, 2H, J=8.7, 6.9 Hz), 3.55-3.61 (t, 1H, J=8.7 Hz), 3.66-3.75 (tt, 1H, J=12, 3.6 Hz), 4.29-4.40 (m, 1H), 4.69-4.76 (dd, 1H, J=9, 6 Hz), 7.08-7.09 (d, 1H, J=5.1 Hz), 7.19-7.20 (m, 1H), 7.30-7.33 (dd, 1H, J=5.1, 2.1 Hz).

### (R)-4-Isobutyl-3-piperidin-4-yl-1-(tetrahydro-pyran-4-yl)-imidazolidin-2-one

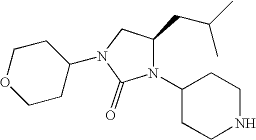

To a cooled (0° C.) solution of 1.0M LAH in THF (76.0 mL, 76.0 mmol) was added D-leucine in one portion, then the mixture was warmed slowly to 60° C. The mixture formed a fluid gel after 2 h. The mixture was cooled to 10° C. and quenched with water (2.88 mL), 4N NaOH (4.33 mL) and water (8.65 mL). Di-t-butyl dicarbonate (15.06 g, 69.0 mmol) was added and the mixture warmed from 11° C. to ambient temperature while stirring for 17 h. The solvent was removed in vacuo and the resulting residue suspended in EtOAc. The solids were removed via filtration and washed with EtOAc. The filtrate was concentrated in vacuo to afford ((R)-1-hydroxymethyl-3-methyl-butyl)-carbamic acid tert-butyl ester (17.0 g, >99%) as a colourless oil. ¹H NMR (CDCl₃) δ 0.92 (d, 6H, J=6.6 Hz), 1.27-1.33 (m, 1H), 1.41-1.50 (m, 10H), 1.58-1.70 (m, 3H), 3.46-3.54 (m, 1H), 3.63-3.75 (m, 2H), 4.49-4.57 (m, 1H).

Following general procedure L, ((R)-1-hydroxymethyl-3-methyl-butyl)-carbamic acid tert-butyl ester (19.81 g, 87.9 mmol) was converted to ((R)-1-aminomethyl-3-methyl-butyl)-carbamic acid tert-butyl ester (10.56 g, 56%). ¹H NMR (CDCl₃) δ 0.92 (d, 6H, J=6.6 Hz), 1.25-1.33 (m, 5H), 1.44 (s, 9H), 1.58-1.69 (m, 1H), 2.55-2.62 (m, 1H), 2.72-2.78 (m, 1H), 3.61 (br s, 1H), 4.44 (br s, 1H).

(R)-4-Isobutyl-3-piperidin-4-yl-1-(tetrahydro-pyran-4-yl)-imidazolidin-2-one (72% over 5 steps) was prepared using the same chemistry as that for (R)-1-cyclohexyl-4-phenyl-3-piperidin-4-yl-imidazolidin-2-one except that ((R)-1-aminomethyl-3-methyl-butyl)-carbamic acid tert-butyl ester was used in lieu of ((R)-2-amino-1-phenyl-ethyl)-carbamic acid tert-butyl ester and tetrahydro-4H-pyranone was used in lieu of cyclohexanone. ¹H NMR (CDCl₃) δ 0.92 (t, 6H, J=6.9 Hz), 1.41 (t, 1H, J=10.8 Hz), 1.55-1.85 (m, 10H), 2.68 (m, 2H), 2.89 (m, 1H), 3.13 (t, 2H, J=12.3 Hz), 3.36 (t, 1H, J=9.0 Hz), 3.47 (t, 2H, J=12.0 Hz), 3.65 (m, 2H), 3.99 (m, 1H), 4.00 (d, 2H, J=9.6 Hz).

### (R)-1-Cyclopentyl-4-isobutyl-3-piperidin-4-yl-imidazolidin-2-one

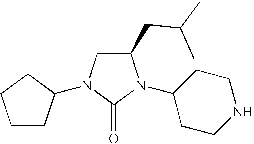

(R)-1-Cyclopentyl-4-isobutyl-3-piperidin-4-yl-imidazolidin-2-one (614 mg, 45% over 5 steps) was prepared using the same chemistry as that for (R)-1-cyclohexyl-4-phenyl-3-piperidin-4-yl-imidazolidin-2-one except that ((R)-1-aminomethyl-3-methyl-butyl)-carbamic acid tert-butyl ester was used in lieu of ((R)-2-amino-1-phenyl-ethyl)-carbamic acid tert-butyl ester and cyclopentanone was used in lieu of cyclohexanone. ¹H NMR (CDCl₃) δ 0.95 (t, 6H, J=5.9 Hz), 1.37-1.90 (m, 16H), 2.63-2.74 (m, 2H), 2.84-2.90 (m, 1H), 3.11-3.20 (m, 1H), 3.35 (t, 1H, J=8.4 Hz), 3.58-3.74 (m, 2H), 4.21-4.30 (m, 1H).

### (R)-4-(2-Fluoro-5-methyl-phenyl)-3-piperidin-4-yl-1-(tetrehydro-pyran-4-yl)-imidazolidin-2-one

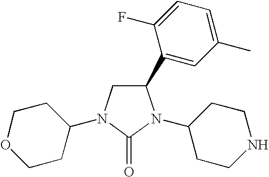

Following general procedure J, 1-fluoro-4-methyl-2-vinyl-benzene (2.200 g, 16.18 mmol) gave [(R)-1-(2-fluoro-5-methyl-phenyl)-2-hydroxy-ethyl]-carbamic acid tert-butyl ester compound (2.39 g, 55%, ee=96%). ¹H NMR (CDCl₃) δ 1.44 (s, 9H), 2.00 (br s, 1H), 2.31 (s, 3H), 3.84 (t, 2H, J=5.7 Hz), 5.00 (br s, 1H), 5.30 (br s, 1H), 6.93 (t, 1H, J=9.3 Hz), 7.03-7.09 (m, 2H).

Following general procedure L, [(R)-1-(2-fluoro-5-methyl-phenyl)-2-hydroxy-ethyl]-carbamic acid tert-butyl ester (5.2 g, 19 mmol) gave [(R)-2-amino-1-(2-fluoro-5-methyl-phenyl)-ethyl]-carbamic acid tert-butyl ester as yellow oil (5.46 g, 100%). ¹H NMR (CDCl₃) δ 1.09 (m, 2H), 1.43 (s, 9H), 2.30 (s, 3H), 2.96-2.99 (m, 2H), 4.81 (br s, 1H), 5.35 (br s 1H), 6.88-6.94 (m, 1H) 7.00-7.06 (m, 2H).

(R)-4-(2-Fluoro-5-methyl-phenyl)-3-piperidin-4-yl-1-(tetrehydro-pyran-4-yl)-imidazolidin-2-one (2.27 g, 34% over 5 steps) was prepared using the same chemistry as that for (R)-1-cyclohexyl-4-phenyl-3-piperidin-4-yl-imidazolidin-2-one except that [(R)-2-amino-1-(2-fluoro-5-methyl)-ethyl]-carbamic acid tert-butyl ester (5.46 g, 19 mmol) was used in lieu of ((R)-2-amino-1-phenyl-ethyl)-carbamic acid tert-butyl ester and tetrahydro-4H-pyranone was used in lieu of cyclohexanone. ¹H NMR (CDCl₃) δ 1.13-1.14 (m, 1H), 1.54-1.71 (m, 8H), 2.30 (s, 3H), 2.52-2.53 (m, 2H), 3.05-3.10 (m, 3H), 3.48-3.49 (m, 2H), 3.65-3.71 (m, 2H), 3.97-4.03 (m, 3H), 4.92-4.97 (m, 1H), 6.89-7.19 (m, 3H).

### 4-[(R)-2-Hydroxy-1-phenyl-ethylamino]-piperidine-1-carboxylic acid tert-butyl ester

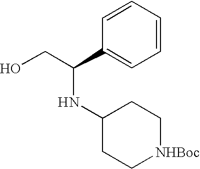

Following general procedure A: a solution of (R)-(−)-2-phenylglycinol (5.021 g, 36.6 mmol), 4-oxo-piperidine-1-carboxylic acid tert-butyl ester (7.66 g, 38.4 mmol), and NaBH(OAc)₃ (10.86 g, 51.3 mmol) in CH₂Cl₂ (140 mL) was stirred at room temperature for 15 h. Standard work-up gave 4-[(R)-2-hydroxy-1-phenyl-ethylamino]-piperidine-1-carboxylic acid tert-butyl ester. ¹H NMR (CDCl₃) δ 1.18-1.33 (m, 2H), 1.44 (s, 9H), 1.63 (m, 1H), 1.89 (m, 1H), 2.07 (br s, 1H), 2.56 (m, 1H), 2.72 (m, 2H), 3.45 (dd, 1H, J=10.5, 9.0 Hz), 3.66 (dd, 1H, J=10.5, 4.5 Hz), 3.91 (dd, 1H, J=9.0, 1.5 Hz), 3.95 (m, 2H), 7.25-7.38 (m, 5H).

### (R)-5-Phenyl-1-piperidin-4-yl-imidazolidin-2-one

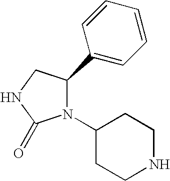

To a solution of [(R)-2-hydroxy-1-phenyl-ethyl]-carbamic acid tert-butyl ester (17.88 g, 75.4 mmol), phthalimide (12.46 g, 83.0 mmol, 1.10 eq), Ph₃P (23.97 g, 90.5 mmol, 1.20 eq) in dry THF (495 mL) at 0° C. was added DEAD (17.65 mL, 83.0 mmol, 1.10 eq) dropwise. The mixture was then stirred at rt for 3 h. The solvent was removed by evaporation under reduced pressure to afford crude 2-[(R)-2-amino-2-phenyl-ethyl]-isoindole-1,3-dione. Following general procedure C, to the above crude carbamate in CH₂Cl₂ (300 mL) at 0° C. was added TFA (60 mL) dropwise. The solution was stirred at rt for 18 h. Standard work-up afforded 2-[(R)-2-amino-2-phenyl-ethyl]-isoindole-1,3-dione as a white solid (14.49 g, 72% over 2 steps).

Following general procedure A, the above amine (13.08 g, 49.1 mmol) was reacted with 1-Boc-4-piperidone (9.79 g, 49.1 mmol) in the presence of NaBH(OAc)₃ (15.33 g, 68.7 mmol) in CH₂Cl₂ (200 mL) for 6 h to give 4-[(R)-2-(1,3-dioxo-1,3-dihydro-isoindol-2-yl)-1-phenyl-ethylamino]-piperidine-1-carboxylic acid tert-butyl ester (22.06 g, 100%).

To a solution of 4-[(R)-2-(1,3-dioxo-1,3-dihydro-isoindol-2-yl)-1-phenyl-ethylamino]-piperidine-1-carboxylic acid tert-butyl ester (22.06 g, 49.1 mmol) in ethanol (300 mL) was added hydrazine hydrate (50 mL). The mixture was stirred at rt for 16 h and at 45° C. for 1 h. Standard work-up gave crude 4-[(R)-2-amino-1-phenyl-ethylamino]-piperidine-1-carboxylic acid tert-butyl ester as a white solid (15.68 g, 100%).

To a solution of the above diamine (15.68 g, 49.1 mmol) in DMF (96 mL) was added carbonyl diimidazole (9.54 g, 58.9 mmol) in portions. The mixture was stirred at rt for 1 h. Standard work-up gave the crude product as a white solid (20.56 g). This was purified by crystallization from EtOAc-hexane to give colorless needles (13.58 g). The impure product from the mother liquid was chromatographed on silica gel (1:1, CH₂Cl₂/EtOAc and 5% MeOH/CH₂Cl₂) to afford another crop of 4-[(R)-2-oxo-5-phenyl-imidazolidin-1-yl]-piperidine-1-carboxylic acid tert-butyl ester (1.41 g, total 14.99 g, 89%).

Following general procedure C, the above carbamate (881 mg, 2.55 mmol) in CH₂Cl₂ (10 mL) was treated with TFA (4 mL) to give (R)-5-phenyl-1-piperidin-4-yl-imidazolidin-2-one (503 mg, 80%). ¹H NMR (CDCl₃) δ 1.03-1.15 (m, 1H), 1.46-1.83 (m, 4H), 2.48 (td, 1H, J=12.3, 2.4 Hz), 2.59 (td, 1H, J=11.7, 2.7 Hz), 2.90 (d, 1H, J=12.3 Hz), 3.07 (d, 1H, J=12.3 Hz), 3.22 (t, 1H, J=7.8 Hz), 3.69 (m, 1H), 3.75 (t, 1H, J=9.0 Hz), 4.59 (br s, 1H), 4.74 (dd, 1H, J=9.3, 6.3 Hz), 7.29-7.39 (m, 5H).

### (R)-2-Oxo-4-phenyl-3-piperidin-4-yl-imidazolidine-1-carboxylic acid ethyl ester

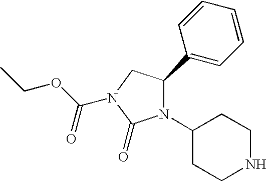

To a solution of 4-[(R)-2-oxo-5-phenyl-imidazolidin-1-yl]-piperidine-1-carboxylic acid tert-butyl ester (2.00 g, 5.79 mmol) in dry THF (30 mL) under N₂ was added NaH (60% dispersion in mineral oil, 463 mg, 11.58 mmol). After stirring at rt for 15 min, the mixture was cooled to 0° C. and ethyl chloroformate (1.70 mL, 17.4 mmol) was added dropwise. The mixture was stirred at rt for 2 h. Aqueous work-up gave crude 4-[(R)-3-ethoxycarbonyl-2-oxo-5-phenyl-imidazolidin-1-yl]-piperidine-1-carboxylic acid tert-butyl ester (2.42 g, 100%).

Using general procedure C, the above carbamate (185 mg, 0.444 mmol) in CH₂Cl₂ (2.5 mL) was treated with TFA (1 mL) to give (R)-2-oxo-4-phenyl-3-piperidin-4-yl-imidazolidine-1-carboxylic acid ethyl ester (141 mg, 100%). ¹H NMR (CDCl₃) δ 1.30 (t, 3H, J=7.2 Hz), 1.37 (m, 1H), 1.53 (d, 1H, J=9.3 Hz), 1.68 (d, 1H, J=10.7 Hz), 2.01 (m, 1H), 1.65 (m, 1H), 2.51-2.65 (m, 2H), 3.00 (d, 1H, J=12.3 Hz), 3.14 (d, 1H, J=12.0 Hz), 3.60 (dd, 1H, J=10.5, 5.4 Hz), 3.64 (m, 1H), 4.11 (t, 1H, J=10.2 Hz), 4.18-4.28 (m, 1H), 4.25 (q, 2H, J=7.2 Hz), 4.64 (dd, 1H, J=9.6, 5.1 Hz), 7.26-7.39 (m, 5H).

### (R)-2-Oxo-4-phenyl-3-piperidin-4-yl-imidazolidine-1-carboxylic acid methyl ester

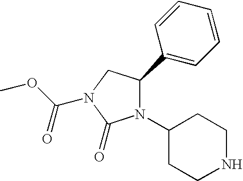

To a solution of 4-[(R)-2-oxo-5-phenyl-imidazolidin-1-yl]-piperidine-1-carboxylic acid tert-butyl ester (1.41 g, 4.09 mmol) in dry THF (23 mL) under N₂ was added NaH (60% dispersion in mineral oil, 196 mg, 4.90 mmol). After stirring at rt for 10 min, the mixture was cooled to 0° C. and methyl chloroformate (379 μL, 4.90 mmol) was added dropwise. The mixture was stirred at rt for 1 h and then at reflux for 2 h. Aqueous work-up and purification provided 4-[(R)-3-methoxycarbonyl-2-oxo-5-phenyl-imidazolidin-1-yl]-piperidine-1-carboxylic acid tert-butyl ester (906 mg, 55%).

Using general procedure C, the above carbamate (694 mg, 1.72 mmol) in CH₂Cl₂ (5 mL) was treated with TFA (2 mL) to give (R)-2-oxo-4-phenyl-3-piperidin-4-yl-imidazolidine-1-carboxylic acid methyl ester (521 mg, 100%). ¹H NMR (CDCl₃) δ 1.13-1.25 (m, 1H), 1.48 (d, 1H, J=12.6 Hz), 1.66 (d, 1H, J=12.0 Hz), 1.76-1.89 (m, 2H), 2.42-2.68 (m, 2H), 2.89 (d, 1H, J=12.6 Hz), 3.05 (d, 1H, J=12.3 Hz), 3.59 (dd, 1H, J=10.5, 5.1 Hz), 3.66 (tt, 1H, J=12.3, 3.9 Hz), 3.82 (s, 3H), 4.10 (t, 1H, J=10.2 Hz), 4.63 (dd, 1H, J=9.6, 4.8 Hz), 7.26-7.37 (m, 5H).

### (R)-2-Oxo-4-phenyl-3-piperidin-4-yl-imidazolidine-1-carboxylic acid dimethylamide

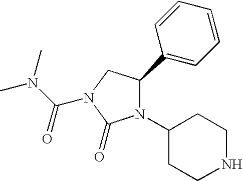

To a solution of 4-[(R)-2-oxo-5-phenyl-imidazolidin-1-yl]-piperidine-1-carboxylic acid tert-butyl ester (500 mg, 1.45 mmol) in dry THF (6 mL) under N₂ was added NaH (60% dispersion in mineral oil, 76 mg, 1.88 mmol). After stirring at rt for 15 min, the mixture was cooled to 0° C. and dimethylcarbamyl chloride (176 μL, 4.90 mmol) was added dropwise. The mixture was heated at reflux for 1 h. Aqueous work-up and purification provided 4-[(R)-3-dimethylcarbamoyl-2-oxo-5-phenyl-imidazolidin-1-yl]-piperidine-1-carboxylic acid tert-butyl ester (597 mg, 99%).

Using general procedure C, the above carbamate (519 mg, 1.25 mmol) in CH₂Cl₂ (5 mL) was treated with TFA (2 mL) to give (R)-2-oxo-4-phenyl-3-piperidin-4-yl-imidazolidine-1-carboxylic acid dimethylamide (331 mg, 84%). ¹H NMR (CDCl₃) δ 1.09-1.47 (d, 1H, J=12.6 Hz), 1.69-1.82 (m, 3H), 2.47 (td, 1H, J=12.3, 2.4 Hz), 2.58 (td, 1H, J=12.0, 3.0 Hz), 2.90 (d, 1H, J=12.3 Hz), 3.03 (s, 6H), 3.07 (d, 1H, J=12.3 Hz), 3.42 (dd, 1H, J=9.9, 4.5 Hz), 3.72 (tt, 1H, J=12.0, 4.2 Hz), 4.11 (t, 1H, J=9.6 Hz), 4.66 (dd, 1H, J=8.7, 4.2 Hz), 7.28-7.37 (m, 5H).

### (R)-2-Oxo-4-phenyl-3-piperidin-4-yl-imidazolidine-1-carboxylic acid methoxy-amide

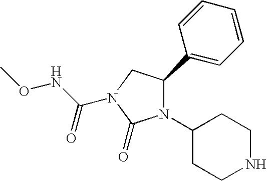

To a solution of 4-[(R)-2-oxo-5-phenyl-imidazolidin-1-yl]-piperidine-1-carboxylic acid tert-butyl ester (438 mg, 1.27 mmol) in dry THF (5 mL) under N₂ was added NaH (60% dispersion in mineral oil, 127 mg, 3.18 mmol). After stirring at rt for 10 min, a solution of methoxycarbamic acid 4-nitrophenyl ester (323 mg, 1.53 mmol) in dry THF (2 mL) was added dropwise. The mixture was stirred at rt for 2 h and then at reflux for 2 h. Standard work-up and purification provided 4-((R)-3-methoxycarbamoyl-2-oxo-5-phenyl-imidazolidin-1-yl)-piperidine-1-carboxylic acid tert-butyl ester (441 mg, 83%).

Using general procedure C, the above product (216 mg, 0.517 mmol) in CH₂Cl₂ (3 mL) was treated with TFA (1 mL) to give (R)-2-oxo-4-phenyl-3-piperidin-4-yl-imidazolidine-1-carboxylic acid methoxy-amide (164 mg, 100%). ¹H NMR (CDCl₃) δ 1.09-1.23 (m, 1H), 1.48 (d, 1H, J=12.3 Hz), 1.66 (d, 2H, J=11.1 Hz), 1.76-1.89 (m, 1H), 2.43-2.60 (m, 2H), 2.92 (d, 1H, J=12.6 Hz), 3.08 (d, 1H, J=12.3 Hz), 3.61 (tt, 1H, J=12.3, 3.9 Hz), 3.66 (dd, 1H, J=10.8, 5.4 Hz), 3.80 (s, 3H), 4.15 (t, 1H, J=10.2 Hz), 4.70 (dd, 1H, J=9.9, 5.4 Hz), 7.26-7.40 (m, 5H), 10.48 (br s, 1H).

### (R)-1-Acetyl-4-phenyl-3-piperidin-4-yl-imidazolidin-2-one

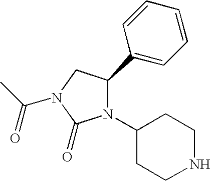

To a solution of 4-[(R)-2-oxo-5-phenyl-imidazolidin-1-yl]-piperidine-1-carboxylic acid tert-butyl ester (345 mg, 1.00 mmol) in dry THF (5 mL) under N₂ was added NaH (60% dispersion in mineral oil, 48 mg, 1.20 mmol). After stirring at rt for 10 min, the mixture was cooled to −78° C. and then acetyl chloride (102 mg, 1.30 mmol) was added dropwise. The mixture was slowly warmed to rt and stirred at rt for 2.5 h. Standard work-up and purification provided 4-[(R)-3-acetyl-2-oxo-5-phenyl-imidazolidin-1-yl]-piperidine-1-carboxylic acid tert-butyl ester (317 mg, 82%).

Using general procedure C, the above product (317 mg, 0.819 mmol) in CH₂Cl₂ (3 mL) was treated with TFA (1.5 mL) to give (R)-1-acetyl-4-phenyl-3-piperidin-4-yl-imidazolidin-2-one (198 mg, 84%). ¹H NMR (CDCl₃) δ 1.04-1.17 (m, 1 H), 1.46 (d, 1 H, J=11.4 Hz), 1.57 (br s, 1H), 1.66 (d, 2H, J=9.3 Hz), 1.71-1.85 (m, 1H), 2.39-2.58 (m, 2H), 2.51 (s, 3H), 2.87 (d, 1H, J=12.3 Hz), 3.04 (d, 1H, J=12.3 Hz), 3.61 (dd, 1H, J=11.7, 5.1 Hz), 3.68 (tt, 1H, J=12.0, 3.9 Hz), 4.08 (dd, 1H, J=11.4, 9.9 Hz), 4.63 (dd, 1H, J=9.6, 4.8 Hz), 7.22-7.33 (m, 5H).

### (R)-2-Oxo-4-phenyl-3-piperidin-4-yl-imidazolidine-1-carboxylic acid methyl amide

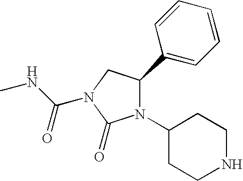

To a solution of 4-[(R)-2-oxo-5-phenyl-imidazolidin-1-yl]-piperidine-1-carboxylic acid tert-butyl ester (345 mg, 1.00 mmol) in dry THF (5 mL) under N₂ was added NaH (60% dispersion in mineral oil, 48 mg, 1.20 mmol). After stirring at rt for 10 min, the mixture was cooled to −78° C. and then a solution of methyl isocyanate (74.1 mg, 1.30 mmol) in dry THF (1 mL) was added dropwise. The mixture was slowly warmed to rt and stirred at rt for 3 h. Standard work-up and purification provided 4-((R)-3-methylcarbamoyl-2-oxo-5-phenyl-imidazolidin-1-yl)-piperidine-1-carboxylic acid tert-butyl ester (217 mg, 54%).

Using general procedure C, the above product (217 mg, 0.540 mmol) in CH₂Cl₂ (2 mL) was treated with TFA (1 mL) to give (R)-2-oxo-4-phenyl-3-piperidin-4-yl-imidazolidine-1-carboxylic acid methyl amide (140 mg, 86%). ¹H NMR (CDCl₃) δ 1.00-1.14 (m, 1H), 1.40 (d, 1H, J=13.8 Hz), 1.45 (s, 1H), 1.58 (d, 1H, J=10.8 Hz), 1.67-1.83 (m, 1H), 2.33-2.51 (m, 2H), 2.79 (d, 1H, J=4.5 Hz), 2.82 (d, 1H, J=12.3 Hz), 2.98 (d, 1H, J=12.3 Hz), 3.54 (tt, 1H, J=12.0, 3.9 Hz), 3.59 (dd, 1H, J=10.8, 5.7 Hz), 4.07 (t, 1H, J=10.5 Hz), 4.58 (dd, 1H, J=9.6, 5.4 Hz), 7.19-7.30 (m, 5H), 7.99 (q, 1H, J=4.5 Hz).

### (R)-1-tert-Butyl-4-phenyl-3-piperidin-4-yl-imidazolidin-2-one

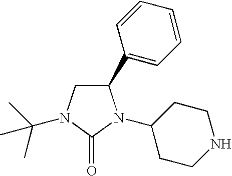

Using general procedure F, tert-butylamine (2.14 g, 20.3 mmol) and ((R)-2-amino-1-phenylethyl)carbamic acid tert-butyl ester (2.55 g, 10.1 mmol) afforded (R)-(tert-butylcarbamoylphenylmethyl)carbamic acid tert-butyl ester (2.88 g, 93%).

Using general procedure C, the above substrate (2.88 g, 9.40 mmol) afforded 2-(R)-amino-N-tert-butyl-2-phenylacetamide (1.94 g, 100%).

To a solution of 2-(R)-amino-N-tert-butyl-2-phenylacetamide (1.94 g, 9.40 mmol) in dry THF was added BH₃.THF (37.0 mL of 1.0 M in THF, 37.6 mmol). This solution was stirred at 60° C. for 18 h. The reaction mixture was then cooled to room temperature. MeOH (10 mL) was added dropwise, then 6N HCl (10 mL) was added. The mixture was stirred at 60° C. for 1 h then the mixture was concentrated under reduced pressure. CH₂Cl₂ (100 mL), saturated aqueous NaHCO₃ (100 mL) and 10N NaOH (4 mL) were added. The aqueous layer was extracted with CH₂Cl₂ (3×100 mL) and the combined organic extracts were dried (MgSO₄), filtered and concentrated under reduced pressure. (R)-N²-tert-Butyl-1-phenylethane-1,2-diamine (1.63 g, 90%) was used in the next step without further purification.

Using general procedure A, the above diamine (1.63 g, 8.05 mmol) and 1-boc-4-piperidone (1.86 g, 9.35 mmol) afforded 4-((R)-2-tert-butylamino-1-phenylethylamino)-piperidine-1-carboxylic acid tert-butyl ester (3.19 g, 100%).

To a solution of the above substrate (3.19 g, 8.05 mmol) and pyridine (1.32 mL, 16.32 mmol) in CH₂Cl₂ (50.0 mL) at 0° C. was added triphosgene (1.21 g, 4.08 mmol). After 1 h, the reaction was quenched with saturated aqueous NaHCO₃ (100 mL) and after work-up and purification afforded 4-((R)-3-tert-butyl-2-oxo-5-phenylimidazolidin-1-yl)-piperidine-1-carboxylic acid tert-butyl ester (2.91 g, 89%).

Using general procedure C, the above substrate (2.91 g, 7.25 mmol) afforded (R)-1-tert-butyl-4-phenyl-3-piperidin-4-yl-imidazolidin-2-one (1.96 g, 90%). ¹H NMR (CDCl₃) δ 1.02 (ddd, 1H, J=24.6, 12.3, 3.9 Hz), 1.36 (s, 9H), 1.42-1.47 (m, 1H), 1.64-1.80 (m, 2H), 2.46 (td, 1H, J=12, 2.4 Hz), 2.54-2.64 (m, 1H), 2.85-2.89 (m, 1H), 3.03-3.11 (m, 2H), 3.63-3.78 (m, 2H), 4.50 (dd, 1H, J=8.4, 7.5 Hz), 7.28-7.35 (m, 5H).

### 4-[4-((R)-3-Cyclohexyl-2-oxo-5-phenyl-imidazolidin-1-yl)-piperidin-1-ylmethyl]-benzoic acid

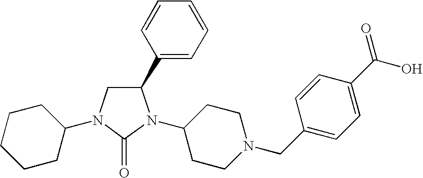

Following general procedure A, (R)-1-cyclohexyl-4-phenyl-3-piperidin-4-yl-imidazolidin-2-one (1.43 g, 4.37 mmol), methyl 4-formylbenzoate (0.79 g, 4.8 mmol) and NaBH(OAc)₃ (1.31 g, 6.18 mmol) afforded a colourless solid (1.33 g, 64%). Following general procedure H, 4-[4-((R)-3-cyclohexyl-2-oxo-5-phenyl-imidazolidin-1-yl)-piperidin-1-ylmethyl]-benzoic acid methyl ester afforded 4-[4-((R)-3-cyclohexyl-2-oxo-5-phenyl-imidazolidin-1-yl)-piperidin-1-ylmethyl]-benzoic acid as a white solid (888 mg, 69%). ¹H NMR (CDCl₃) δ 0.95-1.09 (m, 1H), 1.22-1.47 (m, 5H), 1.60-1.86 (m, 5H), 2.17-2.66 (m, 3H), 2.93-3.20 (m, 3H), 3.40 (br d, 1H, J=11.4 Hz), 3.60-3.95 (m, 5H), 4.54-4.61 (m, 2H), 7.20-7.40 (m, 7H), 7.96 (d, 2H, J=6.9 Hz).

### 4-(4-Bromomethyl-phenoxy)-benzoic acid methyl ester

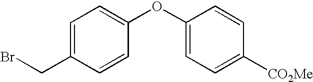

Methyl 4-hydroxybenzoate (7.6 g, 50 mmol) was dissolved in N,N-dimethylacetamide (50 mL). Anhydrous K₃CO₃ (10.4 g, 75 mmol) was added, followed by 4-fluorobenzaldehyde (6.29 g, 50.7 mmol). The mixture was heated to 150° C. and stirred under N₂ for 3 hours. The reaction mixture was cooled to room temperature and was poured onto ˜300 g of crushed ice. The aqueous was carefully adjusted to pH 1-2 with 6 M HCl, and diluted with 500 mL of water. The product was recovered by filtration and dried on high vacuum overnight. The crude material was purified by flash chromatography on silica (hexanes/EtOAc, 15% to 33%) to give 4-(4-formyl-phenoxy)-benzoic acid methyl ester (10.18 g, 80%) as a white powder. ¹H NMR (CDCl₃) δ 3.93 (s, 3H), 7.10 (d, 1H, J=8.4 Hz), 7.14 (d, 1H, J=8.7 Hz), 7.90 (d, 1H, J=8.3 Hz), 8.08 (d, 1H, J=8.4 Hz), 9.96 (s, 1H).

NaBH₄ (253 mg, 6.66 mmol) was dissolved in a mixture of NaOH (1 M, 10 mL) and MeOH (100 mL) at room temperature. The above aldehyde (5.12 g, 20 mmol) was dissolved in a minimum amount of DCM (˜10 mL) and the solution was added drop-wise to the NaBH₄ solution at room temperature with vigorous stirring. TLC showed completed reaction 2 min after the addition. The reaction was quenched by careful addition of 1 M HCl until gas evolvement had stopped and the aqueous pH was ˜3. Most of the volatiles were removed in vacuo and the residue was partitioned between water (100 mL) and DCM (100 mL). The aqueous was extracted with DCM (50 mL×2), and the combined extracts were dried (Na₂SO₄) and concentrated in vacuo to give 4-(4-hydroxymethyl-phenoxy)-benzoic acid methyl ester (5.10 g, 99%) as white crystals.

Bromine (1.26 mL, 24.6 mmol) was added slowly to a cooled (0° C.) mixture of triphenylphosphine (6.45 g, 24.6 mmol) and imidazole (2.29 g, 33.6 mmol) in dry dichloromethane (110 mL) under nitrogen and stirred for 30 min. To the cooled solution was added a solution of the above alcohol (4.88 g, 18.9 mmol) in dry dichloromethane (80 mL) via canula transfer over 5 min. The mixture was stirred at 0° C. for 1 h. Saturated sodium bicarbonate (190 mL) was added and the resulting solution separated. The resulting aqueous layer was extracted with dichloromethane (2×75 mL). The combined organic layers were dried (Na₂SO₄) and concentrated in vacuo. Purification by flash chromatography on silica gel (hexanes/EtOAc, 4:1) afforded 4-(4-bromomethyl-phenoxy)-benzoic acid methyl ester (5.27 g, 86%) as a light yellow solid. ¹H NMR (CDCl₃) δ 3.90 (s, 3H), 4.51 (s, 2H), 6.99-7.03 (m, 4H), 7.39-7.42 (m, 2H), 8.00-8.03 (m, 2H).

### 6-Chloro-2-methyl-pyridine-3-carbaldehyde

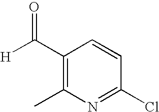

To a 2 L flask was added 2-amino-6-methylpyridine (50 g, 0.462 mole) and DCM (1.0 L) and the solution was cooled to −5° C. N,N-Dibromo-4,4,-dimethylhydantoin (DBH, 66.1 g, 0.231 mol) was added portionwise (6 portions) during a 1 h period while maintaining the pot temperature below −5° C. The reaction was stirred at −5° C. for 1 h after addition, and an aliquot NMR showed about 7% starting material left. Additional DBH was added based on the NMR integration of the remaining starting material. After stirring for another 1 h the mixture was quenched with cold 30% Na₂SO₃ (100 mL) and brine (200 mL). The layers were separated and the aqueous was extracted with CH₂Cl₂ (2×100 mL). The combined organic layers were concentrated to dryness and CH₂Cl₂ (200 mL) was added to the residue followed by hexanes (500 mL). The slurry was agitated for 20 min at rt, then cooled in an ice-water bath for 30 min. The solid was filtered and washed with hexanes to afford 2-amino-6-methyl-5-bromopyridine as white dense crystals (75.22 g, 87%).

To a 2 L 3-neck RBF was added CH₂Cl₂ (900 mL) followed by 2-amino-6-methyl-5-bromopyridine (74.23 g, 0.39 mol), pyridine.HCl (139 g, 1.2 mol), NaNO₂ (83.26 g, 1.2 mol) and CuCl (3.76 g, 5% w/w to starting material). The mixture was cooled to 0-10° C. in an ice-water bath and conc. HCl (4.5 mL, 6% v/w to starting material) was added dropwise and the mixture stirred at 0-10° C. for 30 min. The cooling bath was removed and the mixture was stirred at rt for 1 h. The reaction mixture was quenched with saturated aqueous NaHCO₃ (400 mL), the layers separated and the aqueous layer was extracted with CH₂Cl₂ (100 mL). The combined organic layers were concentrated to dryness and hexanes (750 mL) was added to the residue under stirring. The solid was filtered and washed with hexane and the filtrate was concentrated to dryness to afford pure product as a light yellow crystalline solid (61 g, 70%).

To a 3 L 3-neck flask was added Et₂O followed by 2-chloro-6-methyl-5-bromopyridine (56.97 g, 0.28 mol). The mixture was cooled to −78° C. and n-BuLi (132 mL, 0.33 mol) was added dropwise with an addition funnel while maintaining the pot temperature below −70° C. The mixture was agitated at −78° C. for 30 min, DMF (43 mL, 0.56 mol) was added dropwise at −78° C. then the cooling bath was removed and the reaction was agitated at rt for 1.5 h and quenched with brine (300 mL). The layers were separated and the aqueous layer was extracted with Et₂O (150 mL). The combined organic layers were neutralized with conc. HCl to pH˜3-4 followed by saturated aqueous NaHCO₃ (300 mL). The layers were separated and the organic layer was concentrated to dryness. The product was recrystallized from Et₂O/hexane (200 mL, 1:1) to afford a light yellow crystalline solid (19 g). The filtrate was concentrated and purified by column chromatography (EtOAc/hexane, 1:9) to give another crop of product (13 g). Combined yield was 71%. ¹H NMR (CDCl₃) δ 2.87 (s, 3H), 7.35 (d, 1H, J=9.0 Hz), 8.07 (d, 1H, J=9.0 Hz), 10.31 (s, 1H).

### 6-Bromo-2-methylpyridine-3-carboxaldehyde

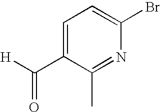

To a solution of 2-amino-6-methylpyridine (10.0 g, 92.5 mmol) in CH₂Cl₂ (200 mL), cooled to −10° C., was added 1,3-dibromo-5,5-dimethylhydantoin (13.2 g, 46.2 mmol) portion-wise. After addition the mixture was brought to room temperature and stirred for 2 h. A saturated aqueous solution of Na₂S₂O₃ (10 mL) and brine (50 mL) were then added and the organic layer was collected. The aqueous layer was extracted with CH₂Cl₂ (4×100 mL). The combined extracts were dried (Na₂SO₄), filtered and concentrated under reduced pressure. The residue was purified by recrystallization from EtOAc/hexanes or by flash chromatography on silica gel (EtOAc/hexanes, 2:3 in v/v) to afford a pale yellow solid (14.5 g, 84%). ¹H NMR (CDCl₃) δ 2.48 (s, 3H), 4.47 (br s, 2H), 6.23 (d, 1H, J=8.4 Hz), 7.47 (d, 1H, J=8.4 Hz).

To a solution of 2-amino-5-bromo-6-methylpyridine (37.6 g, 200 mmol) in aqueous HBr (48%, 200 mL), cooled at 0° C., was added bromine (64.0 g, 400 mmol), forming a yellow suspension. A solution of NaNO₂ (34.5 g, 500 mmol) in water (40 mL) was then added drop-wise. After addition the mixture was brought to room temperature and stirred for 1.5 h, and was poured into ice (200 mL). The aqueous mixture was neutralized with NaOH, and extracted with CH₂Cl₂ (4×100 mL). The combined extracts were dried (Na₂SO₄), filtered and concentrated under reduced pressure. The residue was purified by flash chromatography on silica gel (EtOAc/hexanes, 1:5 in v/v) followed by recrystallization from EtOAc/hexanes, affording a white solid (35.4 g, 71%). ¹H NMR (CDCl₃) δ 2.64 (s, 3H), 7.19 (d, 1H, J=8.4 Hz), 7.63 (d, 1H, J=8.4 Hz).

Under N₂, to a solution of 2,5-dibromo-6-methylpyridine (35.5 g, 141.1 mmol) in anhydrous Et₂O (600 mL), cooled to −78° C., was added BuLi (2.5 M in hexanes, 64.8 mL, 162 mmol) slowly, forming a yellow suspension. After addition the mixture was stirred at that temperature for 1 h, and then anhydrous DMF (18.3 g, 250 mmol) was added. After the mixture was stirred at −78° C. for 1 h, it was brought to room temperature and stirred for an additional 1 h. Aqueous HCl (0.5 N, 300 mL) was added and the organic layer was collected. The aqueous layer was extracted with EtOAc (3×150 mL). The combined extracts were dried (Na₂SO₄), filtered and the solvent was removed. The residue was purified by flash chromatography on silica gel (EtOAc/hexanes, 1:3 in v/v) followed by recrystallization form CH₂Cl₂/hexanes, affording a pale yellow solid (21.5 g, 76%). ¹H NMR (CDCl₃) δ 2.87 (s, 3H), 7.51 (d, 1H, J=8.1 Hz), 7.93 (d, 1H, J=8.1 Hz), 10.30 (s, 1H).

### 6-Bromo-4-methylpyridine-3-carboxaldehyde

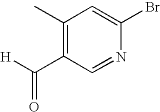

6-Bromo-4-methylpyridine-3-carboxaldehyde was prepared following the same chemistry as for 6-bromo-2-methylpyridine-3-carboxaldehyde except that 2-amino-4-methylpyridine (4.00 g, 37.0 mmol) was used in lieu of 2-amino-6-methylpyridine. 6-Bromo-4-methylpyridine-3-carboxaldehyde was isolated as a pale yellow solid (2.53 g, 35% over 3 steps). ¹H NMR (CDCl₃) δ 2.64 (s, 3H), 7.44 (s, 1H), 8.67 (s, 1H), 10.22 (s, 1H).

### 6-Bromo-5-methylpyridine-3-carboxaldehyde

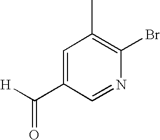

6-Bromo-5-methylpyridine-3-carboxaldehyde was prepared following the same chemistry as for 6-bromo-2-methylpyridine-3-carboxaldehyde except that 2-amino-3-methylpyridine (10.8 g, 100 mmol) was used in lieu of 2-amino-6-methylpyridine. 6-Bromo-5-methylpyridine-3-carboxaldehyde was isolated as a pale yellow solid (2.64 g, 59% over 3 steps). ¹H NMR (CDCl₃) δ 2.49 (s, 3H), 7.96 (d, 1H, J=2.1 Hz), 8.65 (d, 1H, J=8.1 Hz), 10.07 (s, 1H).

### 6-Chloro-2,4-dimethylpyridine-3-carboxaldehyde

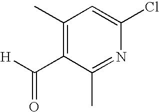

Under N₂, to a solution of 3-bromo-2,4-dimethyl-6-chloropyridine (4.40 g, 20.0 mmol) in anhydrous Et₂O (80 mL), cooled at −78° C., was added tert-BuLi (1.7 M in pentane, 14.0 mL, 24.0 mmol) slowly, forming a yellow suspension. After addition the mixture was stirred at that temperature for 15 min, and then anhydrous DMF (4.0 mL) was added. After the mixture was stirred at −78° C. for 30 min, it was brought to room temperature and stirred for another ½ h. Aqueous work-up and purification by flash chromatography on silica gel (EtOAc/hexanes, 1:4 in v/v) afforded 6-chloro-2,4-dimethylpyridine-3-carboxaldehyde as a pale yellow solid (2.00 g, 60%). ¹H NMR (CDCl₃) δ 2.60 (s, 3H), 2.81 (s, 3H), 7.11 (s, 1H), 10.57 (s, 1H).

### 4-(5-Bromomethyl-pyridin-2-yloxy)-benzonitrile

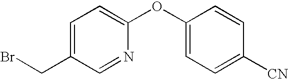

A mixture of 4-chlorophenol (12.0 g, 93.4 mmol), 2-bromo-5-methylpyridine (14.8 g, 86.0 mmol) and K₂CO₃ (20.7 g, 150 mmol) was heated to 200° C. for 5 h. After the mixture was cooled to room temperature water (50 mL) was added and the aqueous mixture was extracted with Et₂O (3×50 mL). The combined extracts were dried (MgSO₄), filtered and concentrated under reduced pressure. The residue was purified by flash chromatography on silica gel (Et₂O/hexanes, 1:6 in v/v) to afford a colorless oil (14.1 g, 75%).

Under N₂, to a dry flask charged with 2-(4-chloro-phenoxy)-5-methyl-pyridine (6.75 g, 30.8 mmol), Zn(CN)₂ (2.35 g, 20.0 mmol), Zn dust (0.400 g, 6.16 mmol), dppf (0.427 g, 0.770 mmol) and Pd₂(dba)₃ (0.284 g, 0.310 mmol) was added dry N,N-dimethylacetamide (40 mL). The mixture was heated to 145° C. for 3 days and then cooled to room temperature. Aqueous ammonia (1N, 50 mL) was added and the mixture was extracted with EtOAc (3×100 mL). The combined extracts were washed with brine (100 mL), dried (Na₂SO₄), filtered and concentrated under reduced pressure. The residue was purified by flash chromatography on silica gel (EtOAc/hexanes, 1:4 in v/v) to afford a pale yellow solid (3.15 g, 49%).

A mixture of 4-(5-methyl-pyridin-2-yloxy)-benzonitrile (0.560 g, 2.69 mmol), NBS (0.958 g, 5.38 mmol) and benzoyl peroxide (0.100 g, 0.413 mmol) in CCl₄ (30 mL) was heated to reflux overnight. After the mixture was cooled to room temperature a solution of Na₂S₂O₃ (1 g) in water (20 mL) was added and the mixture was extracted with CH₂Cl₂ (2×30 mL). The combined organic extract was dried (MgSO₄), filtered and concentrated under reduced pressure. The residue was dissolved in dry THF (10 mL), and diethyl phosphite (0.373 g, 2.70 mmol) and DIPEA (0.348 g, 2.70 mmol) was added. After the mixture was stirred at room temperature for 2 days, a saturated aqueous NaHCO₃ solution (15 mL) was added, and the mixture was extracted with EtOAc (2×20 mL). The combined extracts were dried (Na₂SO₄), filtered and concentrated under reduced pressure. The residue was purified by flash chromatography on silica gel (EtOAc/hexanes, 1:4 in v/v) to afford a pale yellow solid (0.410 g, 53%). ¹H NMR (CDCl₃) δ 4.47 (s, 3H), 7.01 (d, 1H, J=8.4 Hz), 7.22-7.27 (m, 2H), 7.67-7.72 (m, 2H), 7.81 (dd, 1H, J=8.4, 2.4 Hz), 8.18 (d, 1H, J=2.4 Hz).

### 4-(5-Bromomethyl-pyridin-2-ylsulfanyl)-benzoic acid methyl ester

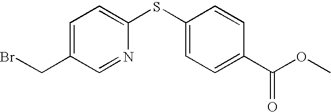

2-Bromo-5-methylpyridine (2.23 g, 13.0 mmol), 4-mercaptobenzoic acid (333 mg, 2.16 mmol), and K₂CO₃ (597 mg, 4.32 mmol) were heated to 200° C. for 2 h. The mixture was partitioned between H₂O (70 mL) and diethyl ether (20 mL). The aqueous phase was extracted with diethyl ether (20 mL) then acidified to pH 3 with 10% HCl(aq). The aqueous phase was extracted with 10% MeOH/CH₂Cl₂ (4×20 mL), and the combined organic layers were dried (MgSO₄) and concentrated to give a yellow solid (412 mg).

A solution of the yellow solid from above (412 mg) and H₂SO₄ (0.11 mL) in MeOH (16 mL) was heated to reflux for 15 h then concentrated. The residue was dissolved in CH₂Cl₂ (15 mL) and washed with H₂O (5 mL) and saturated NaHCO₃(aq) (10 mL) then dried (MgSO₄) and concentrated. Purification by chromatography on silica gel (0%-5% EtOAc/CH₂Cl₂) gave 4-(5-methyl-pyridin-2-ylsulfanyl)-benzoic acid methyl ester as colourless crystals (280 mg, 50% over 2 steps).

A mixture of 4-(5-methyl-pyridin-2-ylsulfanyl)-benzoic acid methyl ester (280 mg, 1.08 mmol), NBS (231 mg, 1.30 mmol), and benzoyl peroxide (39 mg, 0.16 mmol) in CCl₄ (2.7 mL) was heated to reflux for 4 h then filtered and concentrated. Purification by chromatography on silica gel (1% EtOAc/CH₂Cl₂) gave 4-(5-bromomethyl-pyridin-2-ylsulfanyl)-benzoic acid methyl ester as colourless crystals (161 mg, 44%). ¹H NMR (CDCl₃) δ 3.94 (s, 3H), 4.43 (s, 2H), 7.05 (d, 1H, J=8.4 Hz), 7.56 (dd, 1H, J=8.4, 2.4 Hz), 7.61 (dd, 2H, J=6.6, 1.8 Hz), 8.06 (dd, 2H, J=6.8, 1.7 Hz), 8.45 (d, 1H, J=2.1 Hz).

### 4-(5-Formyl-6-methyl-pyridin-2-yloxy)-benzoic acid methyl ester

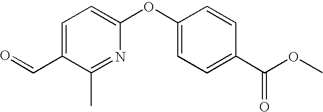

A mixture of 6-bromo-2-methylpyridine-3-carboxaldehyde (4.00 g, 20.0 mmol), 4-hydroxy-benzoic acid methyl ester (3.80 g, 25.0 mmol) and K₂CO₃ (1.73 g, 12.5 mmol) in DMF (30 mL) was heated at 130° C. for 2 h. The mixture was cooled to room temperature and DMF was removed. Aqueous work-up and purification by flash chromatography on silica gel (CH₃OH/CH₂Cl₂, 1:50 in v/v) afforded 4-(5-formyl-6-methyl-pyridin-2-yloxy)-benzoic acid methyl ester as a white solid (3.20 g, 59%). ¹H NMR (CDCl₃) δ 2.72 (s, 3H), 3.93 (s, 3H), 6.86 (d, 1H, J=8.4 Hz), 7.20-7.25 (m, 2H), 8.08-8.15 (m, 3H), 10.25 (s, 1H).

### 4-(5-Formyl-6-methyl-pyridin-2-yloxy)-benzonitrile

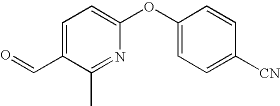

A mixture of 6-bromo-2-methylpyridine-3-carboxaldehyde (1.00 g, 5.00 mmol), 4-hydroxy-benzonitrile (0.596 g, 5.00 mmol) and K₂CO₃ (0.414 g, 3.00 mmol) in DMF (10 mL) was heated at 130° C. for 1 h. The mixture was cooled to room temperature and DMF was removed. Aqueous work-up and purification by flash chromatography on silica gel (EtOAc/hexanes, 2:3 in v/v) afforded 4-(5-formyl-6-methyl-pyridin-2-yloxy)-benzonitrile as a white solid (0.497 g, 41%). ¹H NMR (CDCl₃) δ 2.72 (s, 3H), 6.92 (d, 1H, J=8.4 Hz), 7.28-7.31 (m, 2H), 7.69-7.73 (m, 2H), 8.17 (d, 1H, J=8.4 Hz), 10.26 (s, 1H).

### 4-(5-Formyl-pyridin-2-yloxy)-benzoic acid methyl ester

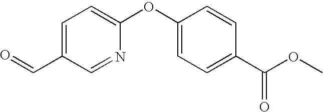

A mixture of 6-bromopyridine-3-carboxaldehyde (3.80 g, 20.4 mmol), 4-hydroxy-benzoic acid methyl ester (4.65 g, 30.6 mmol) and K₂CO₃ (2.48 g, 18.0 mmol) in DMF (30 mL) was stirred at 130° C. for 3 h. The mixture was cooled to room temperature and DMF was removed. Aqueous work-up and purification by flash chromatography on silica gel (CH₃OH/CH₂Cl₂, 1:50 in v/v) afforded 4-(5-formyl-pyridin-2-yloxy)-benzoic acid methyl ester as a white solid (5.00 g, 95%). ¹H NMR (CDCl₃) δ 3.93 (s, 3H), 7.10 (d, 1H, J=8.4 Hz), 7.20-7.26 (m, 2H), 8.10-8.16 (m, 2H), 8.23 (dd, 1H, J=8.4, 2.4 Hz), 8.62 (d, 1H, J=2.4 Hz), 10.00 (s, 1H).

### 4-(5-Formyl-pyridin-2-yloxy)-benzoic acid tert-butyl ester

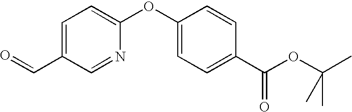

This compound was prepared following the procedure as described for 4-(5-formyl-pyridin-2-yloxy)-benzoic acid methyl ester using 6-bromopyridine-3-carboxaldehyde (2.50 g, 13.4 mmol), 4-hydroxy-benzoic acid tert-butyl ester (2.72 g, 14.0 mmol) and K₂CO₃ (1.10 g, 8.00 mmol) in DMF (25 mL). The product was purified by flash chromatography on basic Al₂O₃ gel (EtOAc/hexanes, 1:5 in v/v) to afford 4-(5-formyl-pyridin-2-yloxy)-benzoic acid tert-butyl ester as a pale yellow oil (3.38 g, 84%). ¹H NMR (CDCl₃) δ 1.60 (s, 9H), 7.19 (d, 1H, J=8.4 Hz), 7.19-7.23 (m, 2H), 8.07-8.11 (m, 2H), 8.22 (dd, 1H, J=8.4, 2.4 Hz), 8.61 (d, 1H, J=2.4 Hz), 9.99 (s, 1H).

### 4-(5-Formyl-4-methyl-pyridin-2-yloxy)-benzoic acid methyl ester

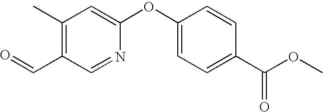

A mixture of 6-bromo-4-methylpyridine-3-carboxaldehyde (1.53 g, 7.69 mmol), 4-hydroxy-benzoic acid methyl ester (1.22 g, 8.0 mmol) and K₂CO₃ (1.06 g, 7.69 mmol) in DMF (20 mL) was stirred at 125° C. for 3 h. The mixture was cooled to room temperature and DMF was removed. Aqueous work-up and purification by flash chromatography on silica gel (CH₃OH/CH₂Cl₂, 1:50 in v/v) afforded 4-(5-formyl-4-methyl-pyridin-2-yloxy)-benzoic acid methyl ester as a white solid (0.524 g, 25%). ¹H NMR (CDCl₃) δ 2.68 (s, 3H), 3.92 (s, 3H), 6.84 (s, 1H), 7.19-7.26 (m, 2H), 8.10-8.14 (m, 2H), 8.50 (s, 1H), 10.11 (s, 1H).

### 4-(5-Formyl-3-methyl-pyridin-2-yloxy)-benzoic acid methyl ester

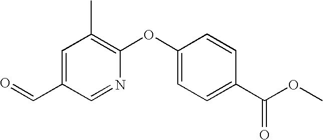

A mixture of 6-bromo-5-methylpyridine-3-carboxaldehyde (1.44 g, 7.24 mmol), 4-hydroxy-benzoic acid methyl ester (1.52 g, 10.0 mmol) and K₂CO₃ (1.00 g, 7.24 mmol) in DMF (20 mL) was stirred at 125° C. for 16 h. The mixture was cooled to room temperature and DMF was removed. Aqueous work-up and purification by flash chromatography on silica gel (EtOAc/hexanes, 1:3 in v/v) afforded 4-(5-formyl-3-methyl-pyridin-2-yloxy)-benzoic acid methyl ester as a white solid (0.98 g, 50%). ¹H NMR (CDCl₃) δ 2.44 (s, 3H), 3.93 (s, 3H), 7.22-7.25 (m, 2H), 8.05 (d, 1H, J=2.1 Hz), 8.11-8.15 (m, 2H), 8.41 (d, 1H, J=2.1 Hz), 9.96 (s, 1H).

### 4-(5-Formyl-4,6-dimethyl-pyridin-2-yloxy)-benzoic acid methyl ester

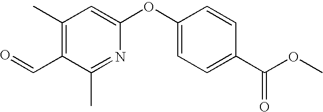

A mixture of 6-chloro-2,4-dimethylpyridine-3-carboxaldehyde (1.40 g, 8.24 mmol), 4-hydroxy-benzoic acid methyl ester (2.49 g, 16.4 mmol) and K₂CO₃ (0.853 g, 6.18 mmol) in DMF (25 mL) was stirred at 125° C. for 2 h. The mixture was cooled to room temperature and DMF was removed. Aqueous work-up and purification by flash chromatography on basic Al₂O₃ gel (EtOAc/hexanes, 1:5 in v/v) followed by recrystallization from CH₂Cl₂/hexanes afforded 4-(5-formyl-4,6-dimethyl-pyridin-2-yloxy)-benzoic acid methyl ester as a white solid (1.16 g, 49%). ¹H NMR (CDCl₃) δ 2.61 (s, 3H), 2.69 (s, 3H), 3.93 (s, 3H), 6.60 (s, 1H), 7.19-7.22 (m, 2H), 8.08-8.11 (m, 2H), 10.52 (s, 1H).

### 4-(5-Formyl-pyridin-2-yloxy)-2-methyl-benzonitrile

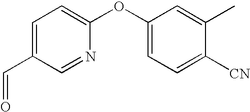

A mixture of 6-chloro-nicotinic acid methyl ester (1.50 g, 8.75 mmol), 4-bromo-3-methylphenol (1.68 g, 9.00 mmol) and K₂CO₃ (1.20 g, 8.70 mmol) in DMF (15 mL) was stirred at 130° C. for 16 h. The mixture was cooled to room temperature, DMF was removed and water (30 mL) was added. The mixture was neutralized with HCl and then extracted with CH₂Cl₂ (3×30 mL). The combined extracts were dried over anhydrous Na₂SO₄. After filtration the solvent was removed, and the residue was purified by flash chromatography on silica gel (CH₂Cl₂) to afford 6-(4-bromo-3-methyl-phenoxy)-nicotinic acid methyl ester as a white solid (2.36 g, 84%).

Under N₂, to a solution of 6-(4-bromo-3-methyl-phenoxy)-nicotinic acid methyl ester (2.36 g, 7.33 mmol) in anhydrous THF (30 mL) cooled to 0° C. was added LAH (1.0 M, THF, 8.0 mL). The mixture was stirred at 0° C. for 30 min and then quenched with water. Brine (30 mL) and saturate aqueous NH₄Cl (10 mL) were added, and the mixture was extracted with EtOAc (3×30 mL). The combined extracts were dried over anhydrous Na₂SO₄. After filtration the solvent was removed, and the residue was dissolved in CH₂Cl₂ (100 mL). MnO₂ (6.30 g, 73.3 mmol) was added, and the suspension was stirred at 40° C. for 2 h. The suspension was then filtered through a Celite® cake. The filtrate was collected and concentrated to afford 6-(4-bromo-3-methyl-phenoxy)-pyridine-3-carbaldehyde as a pale yellow oil (1.59 g, 74%).

Under N₂, to a dry flask charged with 6-(4-bromo-3-methyl-phenoxy)-pyridine-3-carbaldehyde (1.56 g, 5.34 mmol), Zn(CN)₂ (0.451 g, 3.84 mmol), dppf (0.038 g, 0.069 mmol) and Pd₂(dba)₃ (0.025 g, 0.027 mmol) was added anhydrous DMF (15 mL). The mixture was stirred at 140° C. for 16 h and then cooled to room temperature. Aqueous work-up and purification by flash chromatography on silica gel (EtOAc/hexanes, 1:2 in v/v) afforded 4-(5-formyl-pyridin-2-yloxy)-2-methyl-benzonitrile as a pale yellow solid (0.735 g, 58%). ¹H NMR (CDCl₃) δ 2.58 (s, 3H), 7.08-7.14 (m, 3H), 7.67 (d, 1H, J=8.4 Hz), 8.24 (dd, 1H, J=8.4, 2.1 Hz), 8.61 (d, 1H, J=2.1 Hz), 10.01 (s, 1H).

### 4-(5-Formyl-pyridin-2-yloxy)-3-methyl-benzonitrile

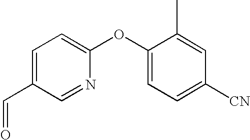

4-(5-Formyl-pyridin-2-yloxy)-3-methyl-benzonitrile was prepared using the same chemistry as for 4-(5-formyl-pyridin-2-yloxy)-2-methyl-benzonitrile except that 4-bromo-2-methylphenol (1.68 g, 9.00 mmol) was used in lieu of 4-bromo-3-methylphenol. 4-(5-Formyl-pyridin-2-yloxy)-3-methyl-benzonitrile was isolated as a pale yellow solid (1.12 g, 52% over 3 steps). ¹H NMR (CDCl₃) δ 2.21 (s, 3H), 7.14 (d, 1H, J=8.4 Hz), 7.19 (d, 1H, J=8.4 Hz), 7.56-7.62 (m, 2H), 8.25 (dd, 1H, J=8.4, 2.1 Hz), 8.57 (d, 1H, J=2.1 Hz), 10.00 (s, 1H).

### 4-(5-Formyl-6-methyl-pyridin-2-ylsulfanyl)-benzoic acid methyl ester

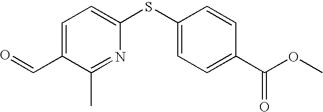

A mixture of 6-bromo-2-methylpyridine-3-carboxaldehyde (0.400 g, 2.00 mmol), 4-mercapto-benzoic acid (0.463 g, 3.00 mmol) and K₂CO₃ (0.414 g, 3.00 mmol) in DMF (5 mL) was stirred at room temperature for 3 h. CH₃I (1.13 g, 8.00 mmol) and another portion of K₂CO₃ (0.414 g, 3.00 mmol) were added, and the mixture was stirred for another 2 h. Aqueous work-up and purification by flash chromatography on silica gel (CH₂Cl₂) afforded 4-(5-formyl-6-methyl-pyridin-2-ylsulfanyl)-benzoic acid methyl ester as a pale yellow solid (0.487 g, 85%). ¹H NMR (CDCl₃) δ 2.81 (s, 3H), 3.96 (s, 3H), 6.85 (d, 1H, J=8.1 Hz), 7.67-7.70 (m, 2H), 7.85 (d, 1H, J=8.1 Hz), 8.09-8.13 (m, 2H), 10.22 (s, 1 H).

### [4-(5-Formyl-6-methyl-pyridin-2-yloxy)-phenyl]-acetic acid methyl ester

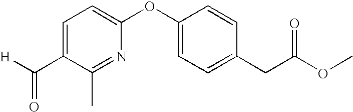

A mixture of 6-chloro-2-methyl-pyridine-3-carbaldehyde (715 mg, 4.60 mmol), methyl 4-hydroxyphenylacetate (694 mg, 4.18 mmol) and K₂CO₃ (404 mg, 2.92 mmol) in DMF (8.4 mL) was heated to 130° C. for 1 h then filtered and concentrated. The residue was dissolved in EtOAc (40 mL) and washed with brine (3×10 mL) then dried (MgSO₄) and concentrated. Purification by chromatography on silica gel (30% EtOAc/hexanes) gave [4-(5-formyl-6-methyl-pyridin-2-yloxy)-phenyl]-acetic acid methyl ester as a yellow oil (687 mg, 58%). ¹H NMR (CDCl₃) δ 2.75 (s, 3H), 3.66 (s, 2H), 3.73 (s, 3H), 6.76 (d, 1H, J=8.7 Hz), 7.13 (d, 2H, J=8.7 Hz), 7.34 (d, 2H, J=8.7 Hz), 8.09 (d, 1H, J=8.4 Hz), 10.24 (s, 1H).

### [4-(5-Formyl-6-methyl-pyridin-2-ylsulfanyl)-phenyl]-acetic acid methyl ester

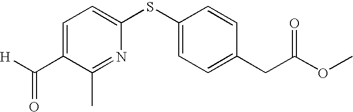

A solution of 4-mercaptophenylacetic acid (985 mg, 5.86 mmol) and H₂SO₄ (0.03 mL) in MeOH (20 mL) was heated to reflux for 45 minutes then concentrated. The residue was dissolved in CH₂Cl₂ (30 mL) and washed with H₂O (10 mL) and saturated NaHCO₃(aq) (10 mL) then dried (MgSO₄) and concentrated to give methyl 4-mercaptophenylacetate as a yellow oil (1.01 g, 94%).

A mixture of 6-chloro-2-methyl-pyridine-3-carbaldehyde (491 mg, 3.16 mmol), methyl 4-mercaptophenylacetate (575 mg, 3.16 mmol), and K₂CO₃ (436 mg, 3.15 mmol) in DMF (6.3 mL) was stirred at room temperature for 2 h then filtered and concentrated. The residue was dissolved in EtOAc (40 mL) and washed with brine (4×10 mL) then dried (MgSO₄) and concentrated to give [4-(5-formyl-6-methyl-pyridin-2-ylsulfanyl)-phenyl]-acetic acid methyl ester as a yellow oil (941 mg, 99%). ¹H NMR (CDCl₃) δ 2.82 (s, 3H), 3.70 (s, 2H), 3.74 (s, 3H), 6.73 (d, 1H, J=8.1 Hz), 7.40 (d, 2H, J=8.1 Hz), 7.58 (d, 2H, J=8.1 Hz), 7.81 (d, 1H, J=8.1 Hz), 10.20 (s, 1H).

### 4-(5-Formyl-6-methyl-pyridin-2-ylsulfanyl)-benzoic acid tert-butyl ester

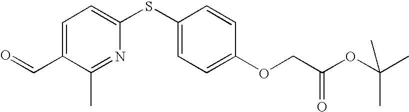

A mixture of 6-chloro-2-methylpyridine-3-carboxaldehyde (3.00 g, 19.3 mmol), 4-mercaptophenol (90% pure, 2.70 g, 19.3 mmol) and K₂CO₃ (1.66 g, 12.0 mmol) in DMF (20 mL) was stirred at room temperature for 16 h. Bromoacetic acid tert-butyl ester (6.00 g, 30.8 mmol) and K₂CO₃ (3.40 g, 24.6 mmol) were added, and the mixture was stirred for another 4 h. Aqueous work-up and purification by flash chromatography on silica gel (EtOAc/hexanes, 1:4 in v/v) afforded 4-(5-formyl-6-methyl-pyridin-2-ylsulfanyl)-benzoic acid tert-butyl ester as a pale yellow solid (7.40 g, 100%). ¹H NMR (CDCl₃) δ 1.50 (s, 9H), 2.81 (s, 3H), 4.57 (s, 2H), 6.66 (d, 1H, J=8.1 Hz), 6.97-7.01 (m, 2H), 7.51-7.55 (m, 2H), 7.79 (d, 1H, J=8.1 Hz), 10.19 (s, 1H).

### 4-(5-Formyl-pyridin-2-ylsulfanyl)-benzoic acid tert-butyl ester

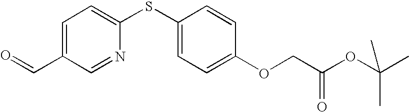

This compound was prepared following the procedure as described for 4-(5-formyl-6-methyl-pyridin-2-ylsulfanyl)-benzoic acid tert-butyl ester using 6-bromopyridine-3-carboxaldehyde (1.80 g, 10.0 mmol). The product was purified by flash chromatography on silica gel (EtOAc/hexanes, 1:4 in v/v) to afford a pale yellow solid (3.20 g, 92%). ¹H NMR (CDCl₃) δ 1.50 (s, 9H), 4.57 (s, 2H), 6.88 (d, 1H, J=8.1 Hz), 6.97-7.01 (m, 2H), 7.51-7.55 (m, 2H), 7.87 (dd, 1H, J=8.1, 2.4 Hz), 8.80 (d, 1H, J=2.4 Hz), 9.96 (s, 1H).

### 3-Fluoro-4-(5-formyl-6-methyl-pyridin-2-yloxy)-benzonitrile

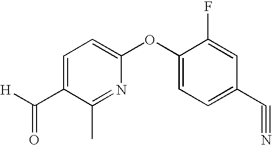

A mixture of 6-bromo-2-methyl-pyridine-3-carbaldehyde (559 mg, 2.79 mmol), 4-bromo-2-fluorophenol (445 mg, 2.33 mmol), and K₂CO₃ (225 mg, 1.63 mmol) in DMF (4.7 mL) was heated to 130° C. for 1 h then filtered and concentrated. The residue was dissolved in EtOAc (40 mL) and washed with brine (4×10 mL) then dried (MgSO₄) and concentrated. Purification by chromatography on silica gel (10% EtOAc/hexanes) gave 6-(4-bromo-2-fluoro-phenoxy)-2-methyl-pyridine-3-carbaldehyde as a yellow oil (472 mg, 65%).

A mixture of 6-(4-bromo-2-fluoro-phenoxy)-2-methyl-pyridine-3-carbaldehyde (472 mg, 1.52 mmol), Zn(CN)₂ (214 mg, 1.82 mmol), Pd₂(dba)₃ (70 mg, 0.076 mmol), and DPPF (84 mg, 0.15 mmol) were heated to 130° C. in degassed DMF (3.0 mL) for 16 h under Argon. The mixture was diluted with EtOAc (15 mL) at room temperature then filtered and concentrated. The residue was dissolved in EtOAc (40 mL) and washed with brine (4×10 mL) then dried (MgSO₄) and concentrated. Purification by chromatography on silica gel (25% EtOAc/hexanes) gave 3-fluoro-4-(5-formyl-6-methyl-pyridin-2-yloxy)-benzonitrile as yellow crystals (202 mg, 52%). ¹H NMR (CDCl₃) δ 2.66 (s, 3H), 7.00 (d, 1H, J=8.4 Hz), 7.38 (m, 1H), 7.51 (m, 2H), 8.18 (d, 1H, J=8.4 Hz), 10.25 (s, 1H).

### 4-(6-Fluoro-5-formyl-pyridin-2-yloxy)-benzoic acid methyl ester

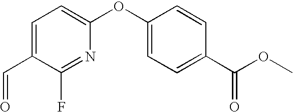

Under N₂, to a solution of 2,6-difluoropyridine (4.95 g, 43.0 mmol) in anhydrous THF (100 mL) cooled at −78° C. was added LDA (2.0 M in heptane/THF/ethylbenzene, 23.0 mL, 46.0 mmol). After the mixture was stirred at −78° C. for 30 min 1-formylpiperidine (4.98 g, 44.0 mmol) was added. The mixture was stirred at −78° C. for 20 min, and at −78° C. aqueous HCl (3 N, 60 mL) and Et₂O (50 mL) were added. The ether layer was collected and the aqueous layer was extracted with Et₂O (3×100 mL). The combined extracts were dried over anhydrous Na₂SO₄. After filtration the solvent was removed, and the residue was purified by flash chromatography on silica gel (CH₂Cl₂/hexanes, 1:1 v/v) to afford 2,6-difluoro-pyridine-3-carbaldehyde as a pale yellow liquid (1.41 g, 60%).

A mixture of 2,6-difluoro-pyridine-3-carbaldehyde (1.10 g, 7.69 mmol), 4-hydroxy-benzoic acid methyl ester (1.17 g, 7.69 mmol) and K₂CO₃ (0.552 g, 4.00 mmol) in DMF (10 mL) was stirred at 100° C. for 2 h. The mixture was cooled to room temperature and DMF was removed. Aqueous work-up and purification by flash chromatography on silica gel (EtOAc/hexanes, 1:4 in v/v) followed by recrystallization from EtOAc/hexanes afforded 4-(6-fluoro-5-formyl-pyridin-2-yloxy)-benzoic acid methyl ester as a white solid (1.48 g, 70%). ¹H NMR (CDCl₃) δ 3.94 (s, 2H), 6.94 (d, 1H, J=8.4 Hz), 7.22-7.26 (m, 2H), 8.12-8.16 (m, 2H), 8.29-8.35 (m, 1H), 10.19 (s, 1H).

### 4-(6-Chloro-5-formyl-pyridin-2-yloxy)-benzoic acid methyl ester

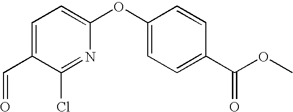

Under N₂, to a solution of 2,6-dichlorpyridine (6.00 g, 40.5 mmol) in anhydrous THF (75 mL) cooled at −78° C. was added LDA (2.0 M in heptane/THF/ethylbenzene, 20.5 mL, 41.0 mmol). After the mixture was stirred at −78° C. for 30 min, 1-formylpiperidine (4.64 g, 41.0 mmol) was added. The mixture was stirred at −78° C. for 20 min, and at −78° C. aqueous HCl (1 N, 60 mL) and Et₂O (50 mL) were added. The organic layer was collected and the aqueous layer was extracted with Et₂O (3×100 mL). The combined extracts were dried over anhydrous Na₂SO₄. After filtration the solvent was removed, and the residue was purified by flash chromatography on silica gel (CH₂Cl₂/hexanes, 1:2 v/v) to afford 2,6-dichloro-pyridine-3-carbaldehyde as a white solid (2.85 g, 40%).

A mixture of 2,6-dichloro-pyridine-3-carbaldehyde (1.53 g, 8.69 mmol), 4-hydroxy-benzoic acid methyl ester (1.37 g, 9.00 mmol) and K₂CO₃ (0.621 g, 4.50 mmol) in DMF (10 mL) was stirred at 120° C. for 2 h. The mixture was cooled to room temperature and DMF was removed. Aqueous work-up and purification by flash chromatography on silica gel (EtOAc/hexanes, 1:4 in v/v) followed by recrystallization from EtOAc/hexanes afforded 4-(6-chloro-5-formyl-pyridin-2-yloxy)-benzoic acid methyl ester as a white solid (1.70 g, 67%). ¹H NMR (CDCl₃) δ 3.94 (s, 2H), 6.98 (d, 1H, J=8.4 Hz), 7.22-7.26 (m, 2H), 8.11-8.15 (m, 2H), 8.25 (d, 1H, J=8.4 Hz), 10.32 (s, 1H).

### 4-(5-Formyl-6-methyl-pyridin-2-ylsulfanyl)-3-methyl-benzonitrile

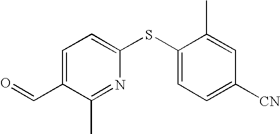

To a suspension of 4-bromo-2-methylaniline (5.40 g, 29.0 mmol) in aqueous (6 N, 14 mL) cooled at 0° C. was added a solution of NaNO₂ (2.27 g, 36.2 mmol) in water (5 mL) slowly. After addition the mixture was stirred at 0° C. for 30 min to give a clear solution. The solution was then added very slowly using a pipette to a solution of O-ethylxanthic acid potassium salt (5.81 g, 36.2 mmol) in water (10 mL) preheated at 40° C. (cautions, potential explosion hazard). After addition the mixture was stirred at 45° C. for 20 min, cooled to room temperature and extracted with Et₂O (3×50 mL). The combined extract was washed with aqueous NaOH (2 N, 40 mL) and water (2×30 mL), and dried over anhydrous Na₂SO₄. After filtration the solvent was removed to give a brown oil. The oil was dissolved in ethanol (30 mL) and heated to 70° C. KOH (7 g) was then added and the mixture was heated at reflux for 16 h. The mixture was then cooled to room temperature, washed with Et₂O (30 mL) and acidified with 6 N HCl to pH=3. Extraction with EtOAc (3×30 mL) was followed by drying over anhydrous Na₂SO₄. After filtration the solvent was removed, and the residue was stirred with 6-chloro-2-methylpyridine-3-carboxaldehyde (2.00 g, 12.8 mmol) and K₂CO₃ (2.00 g, 14.5 mmol) in DMF (20 mL) for 1 h. The mixture was concentrated and aqueous HCl (1 N, 15 mL) and water (20 mL) were added. Extraction with EtOAc (3×30 mL) was performed and the extracts were dried over anhydrous Na₂SO₄. After filtration the solvent was removed, and the residue was purified by flash chromatography on basic Al₂O₃ gel (EtOAc/hexanes, 1:4 in v/v) to afford 6-(4-bromo-2-methyl-phenylsulfanyl)-2-methyl-pyridine-3-carbaldehyde as a yellow oil (2.91 g, 70%).

Under N₂, to a dry flask charged with 6-(4-bromo-2-methyl-phenylsulfanyl)-2-methyl-pyridine-3-carbaldehyde (2.75 g, 8.54 mmol), Zn(CN)₂ (0.587 g, 5.00 mmol), dppf (0.059 g, 0.107 mmol) and Pd₂(dba)₃ (0.039 g, 0.043 mmol) was added anhydrous DMF (40 mL). The mixture was stirred at 135° C. for 16 h and then cooled to room temperature. Aqueous work-up and purification by flash chromatography on silica gel (EtOAc/hexanes, 1:2 in v/v) afforded 4-(5-formyl-6-methyl-pyridin-2-ylsulfanyl)-3-methyl-benzonitrile as a pale yellow solid (0.230 g, 10%). ¹H NMR (CDCl₃) δ 2.44 (s, 3H), 2.78 (s, 3H), 6.78 (d, 1H, J=8.4 Hz), 7.52 (dd, 1H, J=8.1, 1.2 Hz), 7.65 (d, 1H, J=1.2 Hz), 7.70 (d, 1H, J=8.1 Hz), 7.87 (d, 1H, J=8.4 Hz), 10.22 (s, 1H).

### 4-(5-Formyl-6-methyl-pyridin-2-ylsulfanyl)-2-methyl-benzoic acid methyl ester

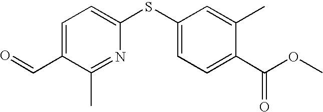

Following the procedure as described for 6-(4-bromo-2-methyl-phenylsulfanyl)-2-methyl-pyridine-3-carbaldehyde using 4-bromo-3-methylaniline (5.40 g, 29.0 mmol), 6-chloro-2-methylpyridine-3-carboxaldehyde (2.60 g, 16.7 mmol) and K₂CO₃ (3.00 g, 21.7 mmol). The product was purified by flash chromatography on silica gel (EtOAc/hexanes, 1:4 in v/v) to afford 6-(4-bromo-3-methyl-phenylsulfanyl)-2-methyl-pyridine-3-carbaldehyde as a pale yellow oil (4.46 g, 83%).

A mixture of 6-(4-bromo-3-methyl-phenylsulfanyl)-2-methyl-pyridine-3-carbaldehyde (2.80 g, 8.33 mmol) and NaBH(OAc)₃ (4.00 g, 18.9 mmol) in CH₂Cl₂ (50 mL) was stirred at room temperature for 24 h. Standard work-up and purification by flash chromatography on silica gel (EtOAc/hexanes 1:1 v/v) afforded [6-(4-bromo-3-methyl-phenylsulfanyl)-2-methyl-pyridin-3-yl]-methanol as a pale yellow solid (2.0 g, 71%).

At 0° C., to a solution of [6-(4-bromo-3-methyl-phenylsulfanyl)-2-methyl-pyridin-3-yl]-methanol (2.00 g, 6.17 mmol) in anhydrous THF (30 mL) was added NaH (60% in mineral oil, 0.48 g, 12 mmol). The mixture was stirred at 0° C. for 10 min, then at room temperature for 30 min. CH₃OCH₂Cl (0.805 g, 10.0 mmol) was then added and the mixture was stirred for 16 h. Standard work-up and purification by flash chromatography on silica gel (EtOAc/hexanes 1:5 v/v) afforded 6-(4-bromo-3-methyl-phenylsulfanyl)-3-methoxymethoxymethyl-2-methyl-pyridine as a pale yellow solid (1.84 g, 81%).

At −78° C., to a solution 6-(4-bromo-3-methyl-phenylsulfanyl)-3-methoxymethoxymethyl-2-methyl-pyridine (1.84 g, 5.00 mmol) in anhydrous THF (30 mL) was added tert-BuLi (1.7 M in pentane, 4.4 mL, 7.5 mL). After addition the mixture was stirred at −78° C. for 15 min, and CO₂ was introduced. After bubbling for 20 min, water (20 mL) was added and the mixture was acidified carefully with 1 N HCl. Extraction with CH₂Cl₂ (10×20 mL) was performed and the combined extracts were dried over anhydrous Na₂SO₄. After filtration the solvent was removed and the residue was dissolved in DMF (15 mL). MeI (1.0 mL, 15 mmol) and K₂CO₃ (1.38 g, 10.0 mmol) were added and the mixture was stirred at room temperature for 5 h. After concentration saturated aqueous NH₄Cl (20 mL) and brine (20 mL) were added and the mixture was extracted with CH₂Cl₂ (4×40 mL). The combined extracts were dried over anhydrous Na₂SO₄. After filtration the solvent was removed, and the residue was purified by flash chromatography on silica gel (EtOAc/hexanes 1:4 v/v) to afford 4-(5-methoxymethoxymethyl-6-methyl-pyridin-2-ylsulfanyl)-2-methyl-benzoic acid methyl ester as a pale yellow oil (0.694 g, 40%).

4-(5-Methoxymethoxymethyl-6-methyl-pyridin-2-ylsulfanyl)-2-methyl-benzoic acid methyl ester (0.694 g, 2.00 mmol) was stirred in aqueous HCl (6 N, 5 mL) and methanol (5 mL) for 30 min. Standard work-up and purification by flash chromatography on silica gel (EtOAc/hexanes 1:1 v/v) afforded 4-(5-hydroxymethyl-6-methyl-pyridin-2-ylsulfanyl)-2-methyl-benzoic acid methyl ester as a pale yellow solid (0.490 g, 78%).

4-(5-Hydroxymethyl-6-methyl-pyridin-2-ylsulfanyl)-2-methyl-benzoic acid methyl ester (0.490 g, 1.56 mmol) and MnO₂ (3 g) were stirred in CH₂Cl₂ (30 mL) for 16 h. The suspension was then filtered through a Celite® cake and the solvent was removed to afford 4-(5-formyl-6-methyl-pyridin-2-ylsulfanyl)-2-methyl-benzoic acid methyl ester as a pale yellow solid (0.380 g, 78%). ¹H NMR (CDCl₃) δ 2.62 (s, 3H), 2.81 (s, 3H), 3.93 (s, 3H), 6.82 (d, 1H, J=8.1 Hz), 7.46-7.51 (m, 2H), 7.84 (d, 1H, J=8.4 Hz), 7.97 (d, 1H, J=8.1 Hz), 10.22 (s, 1H).

### 4-(5-Formyl-pyrimidin-2-yloxy)-benzoic acid methyl ester

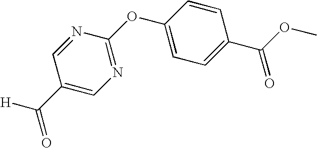

To a solution of methyl 4-hydroxybenzoate (260 mg, 1.71 mmol) in DMF (25 mL) at rt was added NaH (60%, 75 mg, 1.9 mmol) and the mixture was stirred for 30 minutes. 5-Bromo-2-chloropyrimidine (300 mg, 1.55 mmol) was added and the mixture heated to 130° C. for 0.5 hours. Standard work-up and purification afforded 4-(5-bromo-pyrimidin-2-yloxy)-benzoic acid methyl ester (428 mg, 89%).

To a solution of the above pyrimidine (428 mg, 1.38 mmol), bis(triphenylphosphine)palladium(II) dichloride (145 mg, 0.207 mmol) and LiCl (289 mg, 6.9 mmol) in degassed DMF (5 mL) was added tributyl(vinyl)tin (0.48 mL, 1.64 mmol) and the mixture was heated at 100° C. overnight. Standard work-up and purification afforded 4-(5-vinyl-pyrimidin-2-yloxy)-benzoic acid methyl ester (210 mg, 59%).

To a solution of the above substrate (210 mg, 0.819 mmol) and NMO (297 mg, 2.45 mmol) in CH₂Cl₂ (8 mL) was added OsO₄ (2.5%, 0.13 mL) and the mixture stirred at room temperature overnight. Standard work-up afforded the crude intermediate (127 mg). To a solution of the diol in acetone (2 mL) was added a solution of NaIO₄ (138 mg, 0.972 mmol) in H₂O (1 mL) and the mixture was stirred at room temperature for 2 hours. Standard work-up and purification afforded 4-(5-formyl-pyrimidin-2-yloxy)-benzoic acid methyl ester (87 mg, 37% over 2 steps). ¹H NMR (CDCl₃) δ 3.94 (s, 3H), 7.29 (d, 2H, J=9 Hz), 8.16 (d, 2H, J=9 Hz), 9.03 (s, 2H), 10.06 (s, 1H).

### 4-(5-Formyl-pyrimidin-2-ylsulfanyl)-benzoic acid methyl ester

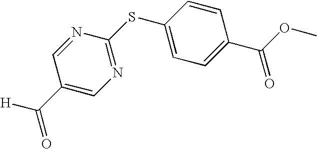

To a solution of 4-mercaptobenzoic acid (182 mg, 1.18 mmol) in DMF (10 mL) at rt was added NaH (60%, 104 mg, 2.59 mmol) and the mixture was stirred for 30 minutes. 5-Bromo-2-chloropyrimidine (228 mg, 1.18 mmol) was added and the mixture heated to 130° C. for 3 hours. Standard work-up and purification afforded the desired acid which was treated with H₂SO₄ (0.2 mL) in MeOH (5 mL) to afford 4-(5-bromo-pyrimidin-2-ylsulfanyl)-benzoic acid methyl ester (370 mg, quant).

To a solution of the above pyrimidine (370 mg, 1.13 mmol), bis(triphenylphosphine)palladium(II) dichloride (118 mg, 0.170 mmol), LiCl (189 mg) and triphenylphosphine (89 mg, 0.34 mmol) in degassed DMF (5 mL) was added tributyl(vinyl)tin (0.40 mL, 1.4 mmol) and the mixture was heated at 100° C. overnight. Standard work-up and purification afforded 4-(5-vinyl-pyrimidin-2-ylsulfanyl)-benzoic acid methyl ester (78 mg, 25%).

To a solution of the above substrate (79 mg, 0.29 mmol) and NMO (67 mg, 0.57 mmol) in CH₂Cl₂ (3 mL) was added OsO₄ (2.5%, 0.05 mL, 0.004 mmol) and the mixture stirred at room temperature overnight. Standard work-up afforded the crude intermediate. To a solution of the diol in acetone (2 mL) was added a solution of NaIO₄ (91 mg, 0.58 mmol) in H₂O (1 mL) and the mixture was stirred at room temperature for 2 hours. Standard work-up and purification afforded 4-(5-formyl-pyrimidin-2-ylsulfanyl)-benzoic acid methyl ester (40 mg, 50% over 2 steps). ¹H NMR (CDCl₃) δ 3.94 (s, 3H), 7.69 (d, 2H, J=8.4 Hz), 8.10 (d, 2H, J=8.1 Hz), 8.87 (s, 2H), 9.99 (s, 1H).

### 4-(5-Formyl-4-methyl-pyrimidin-2-ylamino)-benzonitrile

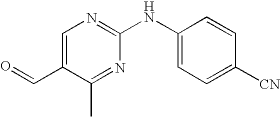

To anhydrous ethanol (200 mL) was added a small sodium chip (1.08 g, 47.0 mmol). The mixture was stirred until all the metal disappeared. To the clear solution was then added guanidine hydrochloride (4.78 g, 50.0 mmol), forming a white suspension. 2-[1-Dimethylamino-methylidene]-3-oxo-butyric acid ethyl ester (Tetrahedron Letter 39, 1998, 213-216) (7.40 g, 40.0 mmol) was then added, immediately forming a yellow thick suspension. The suspension was stirred at room temperature for 1 h, and then concentrated to dryness. The residue was collected, washed with water thoroughly and dried to afford 2-amino-4-methyl-pyrimidine-5-carboxylic acid ethyl ester as a white solid (6.20 g, 86%).

Under N₂, to a dry flask charged with 2-amino-4-methyl-pyrimidine-5-carboxylic acid ethyl ester (2.50 g, 13.8 mmol), 1,4-dibromobenzene (6.51 g, 27.6 mmol), tert-BuOK (2.02 g, 18.0 mmol), 4,5-bis(diphenylphosphino)-9,9-dimethylxanthene (0.176 g, 0.304 mmol) and Pd₂(dba)₃ (0.126 g, 0.138 mmol) was added anhydrous toluene (100 mL). The mixture was degassed and filled with N₂ twice, and then was stirred at 100° C. for 16 h. After the mixture was cooled to room temperature, saturated aqueous NH₄Cl solution (20 mL) and brine (50 mL) were added and the mixture was extracted with EtOAc/THF (3×60 mL, 5:1 v/v). The combined extracts were dried over anhydrous Na₂SO₄. After filtration through a silica gel plug the solvent was removed, and the residue was purified by recrystallization from EtOAc/hexanes to afford 2-(4-bromo-phenylamino)-4-methyl-pyrimidine-5-carboxylic acid ethyl ester as a pale yellow solid (2.80 g, 60%).

Under N₂, to a solution of 2-(4-bromo-phenylamino)-4-methyl-pyrimidine-5-carboxylic acid ethyl ester (2.80 g, 8.33 mmol) in anhydrous THF (100 mL) cooled at −78° C. was added DIBAL-H (1.0 M, toluene, 21.0 mL, 21.0 mmol). After the mixture was stirred at −78° C. for 30 min the cooling bath was removed and the mixture was stirred at room temperature for 2 h. Saturated aqueous NH₄Cl (60 mL) was added and the mixture was extracted with EtOAc (3×50 mL). The combined extracts were dried over anhydrous Na₂SO₄. After filtration the solvent was removed, and the residue was purified by flash chromatography on silica gel (EtOAc) to afford [2-(4-bromo-phenylamino)-4-methyl-pyrimidin-5-yl]-methanol as a pale yellow solid (2.00 g, 82%).

[2-(4-Bromo-phenylamino)-4-methyl-pyrimidin-5-yl]-methanol (2.00 g, 6.85 mmol) was dissolved in CH₂Cl₂ (200 mL). MnO₂ (15.0 g, 174 mmol) was added, and the suspension was stirred at room temperature for 4 h. The suspension was then filtered through a Celite® cake. The filtrate was collected and concentrated to afford 2-(4-bromo-phenylamino)-4-methyl-pyrimidine-5-carbaldehyde as a pale yellow solid (1.49 g, 75%).

Under N₂, to a dry flask charged with 2-(4-bromo-phenylamino)-4-methyl-pyrimidine-5-carbaldehyde (1.52 g, 5.21 mmol), Zn(CN)₂ (0.352 g, 3.00 mmol), dppf (0.036 g, 0.065 mmol) and Pd₂(dba)₃ (0.024 g, 0.026 mmol) was added anhydrous DMF (40 mL). The mixture was stirred at 140° C. for 16 h and then cooled to room temperature. DMF was removed, the residue was taken up with EtOAc and purified by flash chromatography on silica gel (EtOAc/hexanes, 1:1 in v/v) to afford 4-(5-formyl-4-methyl-pyrimidin-2-ylamino)-benzonitrile as a yellow solid (0.711 g, 57%). ¹H NMR (CDCl₃) δ 2.79 (s, 3H), 7.64 (br s, 1H), 7.64-7.68 (m, 2H), 7.85-7.88 (m, 2H), 8.79 (s, 1H), 10.09 (s, 1H).

### [4-(5-Formyl-6-methyl-pyridin-2-yloxy)-phenoxy]-acetic acid tert-butyl ester

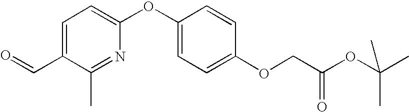

To a solution of hydroquinone (4.88 g, 44.4 mmol) and KOH (85% pure, 4.65 g, 66.6 mmol) in water (20 mL)/1,4-dioxane (50 mL) was added bromoacetic acid tert-butyl ester (8.66 g, 44.4 mmol), and the mixture was stirred at room temperature for 1 h. Acidic work-up and purification by flash chromatography on silica gel (EtOAc/hexanes, 1:2 in v/v) followed by recrystallization from EtOAc/hexanes afforded (4-hydroxy-phenoxy)-acetic acid tert-butyl ester as a white solid (5.25 g, 53%). ¹H NMR (CD₃OD) δ 1.48 (s, 9H), 4.50 (s, 2H), 6.71-6.81 (m, 4H).

A mixture of 6-bromo-2-methylpyridine-3-carboxaldehyde (2.20 g, 11.0 mmol), (4-hydroxy-phenoxy)-acetic acid tert-butyl ester (2.47 g, 11.0 mmol) and K₂CO₃ (0.911 g, 6.60 mmol) in DMF (25 mL) was stirred at 130° C. for 1.5 h. The mixture was cooled to room temperature and DMF was removed. Aqueous work-up and purification by flash chromatography on basic Al₂O₃ gel (EtOAc/hexanes, 1:4 in v/v) followed by recrystallization from CH₂Cl₂/hexanes afforded [4-(5-formyl-6-methyl-pyridin-2-yloxy)-phenoxy]-acetic acid tert-butyl ester as a pale yellow solid (2.05 g, 54%). ¹H NMR (CDCl₃) δ 1.50 (s, 9H), 2.74 (s, 3H), 4.53 (s, 2H), 6.71 (d, 1H, J=8.4 Hz), 6.92-6.97 (m, 2H), 7.06-7.10 (m, 2H), 8.07 (d, 1H, J=8.4 Hz), 10.23 (s, 1H).

### [4-(5-Formyl-6-methyl-pyridin-2-yloxy)-phenylsulfanyl]-acetic acid tert-butyl ester

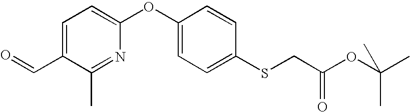

A mixture of 4-mercaptophenol (90% pure, 2.30 g, 16.4 mmol), bromoacetic acid tert-butyl ester (3.90 g, 20.0 mmol) and K₂CO₃ (1.38 g, 10.0 mmol) in DMF (20 mL) was stirred at room temperature for 3 h. Aqueous work-up and purification by flash chromatography on silica gel (EtOAc/hexanes, 1:3 in v/v) afforded (4-hydroxy-phenylsulfanyl)-acetic acid tert-butyl ester as a colorless oil (2.92 g, 77%). ¹H NMR (CDCl₃) δ 1.41 (s, 9H), 3.42 (s, 2H), 6.76-6.79 (m, 2H), 7.34-7.37 (m, 2H).

A mixture of 6-chloro-2-methylpyridine-3-carboxaldehyde (1.87 g, 12.0 mmol), (4-hydroxy-phenylsulfanyl)-acetic acid tert-butyl ester (2.48 g, 10.7 mmol) and K₂CO₃ (0.830 g, 6.00 mmol) in DMF (20 mL) was heated at 125° C. for 1 h. The mixture was cooled to room temperature and DMF was removed. Aqueous work-up and purification by flash chromatography on basic Al₂O₃ gel (CH₂Cl₂/hexanes, 1:1 in v/v) afforded [4-(5-formyl-6-methyl-pyridin-2-yloxy)-phenylsulfanyl]-acetic acid tert-butyl ester as a pale yellow solid (0.481 g, 13%). ¹H NMR (CDCl₃) δ 1.42 (s, 9H), 2.72 (s, 3H), 3.56 (s, 2H), 6.78 (d, 1H, J=8.4 Hz), 7.09-7.13 (m, 2H), 7.46-7.50 (m, 2H), 8.11 (d, 1H, J=8.4 Hz), 10.24 (s, 1H).

### 6-(4-Methoxy-phenoxy)-2-methyl-pyridine-3-carbaldehyde

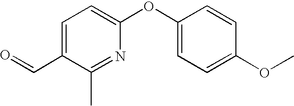

A mixture of 6-bromo-2-methylpyridine-3-carboxaldehyde (1.00 g, 5.00 mmol), 4-hydroxy-benzonitrile (0.620 g, 5.00 mmol) and K₂CO₃ (0.414 g, 3.00 mmol) in DMF (10 mL) was heated at 130° C. for 1 h. The mixture was cooled to room temperature and DMF was removed. Aqueous work-up and purification by flash chromatography on silica gel (EtOAc/hexanes, 1:3 in v/v) afforded 6-(4-methoxy-phenoxy)-2-methyl-pyridine-3-carbaldehyde as a yellow oil (0.966 g, 80%). ¹H NMR (CDCl₃) δ 2.75 (s, 3H), 3.84 (s, 3H), 6.70 (d, 1H, J=8.4 Hz), 6.92-6.97 (m, 2H), 7.06-7.10 (m, 2H), 8.06 (d, 1H, J=8.4 Hz), 10.23 (s, 1H).

### 6-(4-Methoxy-phenylsulfanyl)-2-methyl-pyridine-3-carbaldehyde

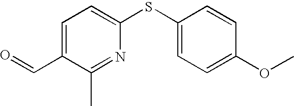

To a solution of 6-bromo-2-methyl-pyridine-3-carbaldehyde (1.005 g, 5.026 mmol), and 4-methoxy-benzenethiol (0.726 g, 5.026 mmol) in DMF (10 mL) was added K₂CO₃ (0.416 g, 3.015 mmol). The mixture was stirred at rt for 24 h. DMF was evaporated under reduced pressure and the residue was taken up in water (20 mL) and neutralized with 1 N HCl to pH˜6. The solution was extracted with CH₂Cl₂ (3×30 mL). The combined extracts were dried (Na₂SO₄), filtered and the solvent was evaporated to give the crude product. This was purified by column chromatography (6:1, hexane/EtOAc) to give 6-(4-methoxy-phenylsulfanyl)-2-methyl-pyridine-3-carbaldehyde (1.095 g, 84%). ¹H NMR (CDCl₃) δ 2.81 (s, 3H), 3.87 (s, 3H), 6.65 (d, 1H, J=8.4 Hz), 7.00 (d, 2H, J=9.0 Hz), 7.53 (d, 2H, J=9.0 Hz), 7.79 (d, 1H, J=8.4 Hz), 10.19 (s, 1H).

### N-Cyclopropyl-4-(5-formyl-pyridin-2-yloxy)-benzamide

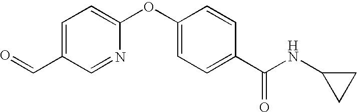

Following general procedure E: a mixture of 4-hydroxybenzoic acid (2.76 g, 20.0 mmol), cyclopropyl amine (1.71 g, 30.0 mmol), EDCI (4.80 g, 25.0 mmol), HOBT (3.38 g, 25.0 mmol) and DIPEA (3.87 g, 30.0 mmol) in DMF (20 mL) was stirred for 16 h affording N-cyclopropyl-4-hydroxy-benzamide as a white solid (3.30 g, 93%).

A mixture of 6-chloropyridine-3-carboxaldehyde (8.85 g, 62.5 mmol), N-cyclopropyl-4-hydroxy-benzamide (11.1 g, 62.5 mmol) and K₂CO₃ (5.18 g, 37.5 mmol) in DMF (120 mL) was stirred at 100° C. for 1.5 h. The mixture was cooled to room temperature and DMF was removed. Methanol (30 mL) and water (50 mL) were added and the mixture was stirred for 10 min. Methanol was then removed and the residual mixture was extracted with CH₂Cl₂ (3×100 mL). The combined extracts were washed with dilute aqueous K₂CO₃ solution (5 g) in water (50 mL) twice and water (50 mL), and dried over anhydrous Na₂SO₄. After filtration the solvent was removed, and the residue was washed with CH₂Cl₂/hexanes (2:5 in v/v) to afford N-cyclopropyl-4-(5-formyl-pyridin-2-yloxy)-benzamide as a white solid (15.1 g, 86%). ¹H NMR (CDCl₃) δ 0.59-0.65 (m, 2H), 0.86-0.92 (m, 2H), 2.88-2.94 (m, 1H), 6.28 (br s, 1H), 7.08 (d, 1H, J=8:4 Hz), 7.20-7.24 (m, 2H), 7.81-7.84 (m, 2H), 8.22 (dd, 1H, J=8.4, 2.4 Hz), 8.60 (d, 1H, J=2.4 Hz), 9.99 (s, 1H).

### N-Cyclopropyl-4-(5-formyl-6-methyl-pyridin-2-yloxy)-benzamide

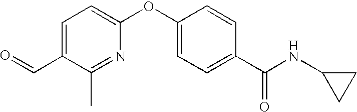

A mixture of 2-methyl-6-chloropyridine-3-carboxaldehyde (0.850 g, 4.25 mmol), N-cyclopropyl-4-hydroxy-benzamide (0.765 g, 4.32 mmol) and K₂CO₃ (0.414 g, 2.55 mmol) in DMF (8 mL) was stirred at 105° C. for 2 h. The mixture was cooled to room temperature and DMF was removed. Methanol (20 mL) and water (20 mL) were added and the mixture was stirred for 10 min. Methanol was then removed and the residual mixture was extracted with CH₂Cl₂ (3×40 mL). The combined extracts were dried over anhydrous Na₂SO₄. After filtration the solvent was removed, and the residue was purified by flash chromatography on silica gel (MeOH/CH₂Cl₂, 1:50 v/v) followed by recrystallization from EtOAc/hexanes, affording N-cyclopropyl-4-(5-formyl-6-methyl-pyridin-2-yloxy)-benzamide as a white solid (0.79 g, 63%). ¹H NMR (CDCl₃) δ 0.60-0.66 (m, 2H), 0.86-0.92 (m, 2H), 2.71 (s, 3H), 2.88-2.95 (m, 1H), 6.23 (br s, 1H), 6.84 (d, 1H, J=8.7 Hz), 7.18-7.23 (m, 2H), 7.77-7.82 (m, 2H), 8.13 (d, 1H, J=8.7 Hz), 10.24 (s, 1H).

Examples 1 to 9 were prepared following the scheme illustrated below. RCHO is as defined in the table.

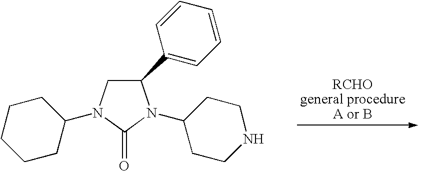

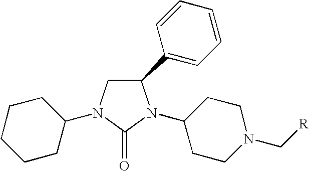

| Example | RCHO |
|---|---|
| 1 | 4-pyrimidin-5-yl-benzaldehyde |
| 2 | 6-pyrimidin-5-yl-pyridine-3-carbaldehyde |
| 3 | 4-formyl-N-isopropyl-benzamide |
| 4 | 5-methyl-1-phenyl-1H-pyrazole-4-carbaldehyde |
| 5 | 4-formyl-N-isopropyl-benzenesulfonamide |
| 6 | 1-oxo-2,3-dihydro-1H-isoindole-5-carbaldehyde |
| 7 | 5-formylpyridine-2-carboxylic acid iso-propylamide |
| 8 | 5-formyl-2-hydroxy-N-isopropyl-benzamide |
| | (see EXAMPLE 12) |
| 9 | imidazo[1,2-a]pyridine-6-carbaldehyde |
| | (Eisai Co., U.S. Patent No. 5444066 A1) |

### EXAMPLE 1

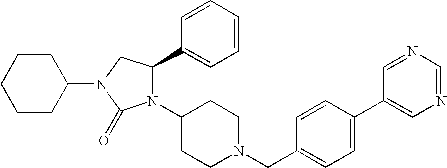

### COMPOUND 1: (R)-1-Cyclohexyl-4-phenyl-3-[1-(4-pyrimidin-5-yl-benzyl)-piperidin-4-yl]-imidazolidin-2-one

Using general procedure D, 5-bromopyrimidine (199 mg, 1.25 mmol) and 4-formylbenzene boronic acid (469 mg, 3.13 mmol) afforded 4-pyrimidin-5-yl-benzaldehyde (162 mg, 99%).

COMPOUND 1 was isolated as a white solid (40 mg, 68%). ¹H NMR (CDCl₃) δ 0.88-2.20 (m, 16H), 2.70 (d, 1H, J=11.4 Hz), 2.88 (d, 1H, J=10.4 Hz), 3.03 (dd, 1H, J=8.7, 6.9 Hz), 3.39-3.51 (m, 2H), 3.58-3.83 (m, 3H), 4.57 (dd, 1H, J=9.1, 6.6 Hz), 7.27-7.35 (m, 5H), 7.38 (d, 2H, J=8.3 Hz), 7.48 (d, 2H, J=8.1 Hz,), 8.91 (s, 2H), 9.17 (s, 1H); ¹³C NMR (CDCl₃) δ 26.0, 29.4, 30.3, 30.8, 31.3, 48.9, 51.7, 52.5, 53.6, 53.8, 56.5, 62.7, 127.1, 128.4, 129.2, 130.4, 132.4, 133.2, 134.6, 140.2, 143.6, 155.2, 157.7, 160.6; ES-MS m/z 496 (M+1).

### EXAMPLE 2

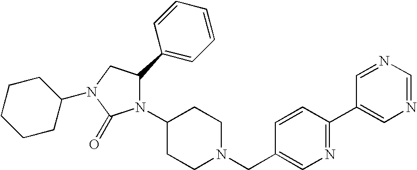

### COMPOUND 2: (R)-1-Cyclohexyl-4-phenyl-3-[1-(6-pyrimidin-5-yl-pyridin-3-ylmethyl)-piperidin-4-yl]-imidazolidin-2-one

To a solution of methyl 6-chloronicotinate (0.60 g, 3.50 mmol) in THF (10 mL) at −78° C. was added a solution of DIBAL-H (1 M in toluene, 10.5 mL, 10.5 mmol) and the reaction stirred from −78° C. to room temperature for 1 h. The reaction was diluted with a saturated aqueous solution of sodium potassium tartrate (25 mL) and CH₂Cl₂ (30 mL) and stirred vigorously overnight. The layers were separated and the aqueous layer was extracted with CH₂Cl₂ (2×15 mL). The combined organic extracts were dried (Na₂SO₄) and concentrated to afford the desired alcohol (0.48 g, 95%) as a white solid.

To a solution of the alcohol from above (481 mg, 3.35 mmol) in CHCl₃ (25 mL) was added MnO₂ (85%, 3.14 g, 30.7 mmol) and the suspension stirred at 60° C. overnight. The reaction was cooled, filtered through Celite®, washing the cake with CH₂Cl₂ and MeOH and the resultant filtrate concentrated to afford the desired aldehyde (0.41 g, 87%) as a yellow solid. To a solution of 6-chloro-3-pyridinecarboxaldehyde (210 mg, 1.48 mmol) and pyrimidine-5-boronic acid (207 mg, 1.67 mmol) in THF/DME/2 M Na₂CO₃ (1:2:1, 4 mL) was added Pd(PPh₃)₄ (154 mg, 0.13 mmol) and the reaction stirred under argon at 90° C. overnight. Standard work-up and purification by column chromatography on silica gel (CH₂Cl₂/MeOH, 95:5) afforded 6-pyrimidin-5-yl-pyridine-3-carbaldehyde (80 mg, 29%) as a yellow solid. ¹H NMR (CDCl₃) δ 7.96 (d, 1H, J=8.1 Hz), 8.32 (dd, 1H, J=8.1, 1.5 Hz), 9.20 (d, 1H, J=1.5 Hz), 9.32 (s, 1H), 9.42 (s, 2H), 10.18 (s, 1H).

COMPOUND 2 was isolated as a white foam (66 mg, 68%). ¹H NMR (CDCl₃) δ 1.01-1.04 (m, 1H), 1.21-1.40 (m, 6H), 1.61-1.76 (m, 6H), 1.90-2.07 (m, 3H), 2.66-2.71 (m, 1H), 2.84-2.89 (m, 1H), 3.04 (dd, 1H, J=8.7, 7.2 Hz), 3.42-3.51 (m, 2H), 3.60-3.80 (m, 3H), 4.57 (dd, 1H, J=9, 6 Hz), 7.29-7.34 (m, 5H), 7.67 (d, 1H J=7.8 Hz), 7.73 (dd, 1H, J=8.1, 1.8 Hz), 8.58 (d, 11H, J=1.2 Hz), 9.23 (s, 1H), 9.29 (s, 2H); ¹³C NMR (CDCl₃) δ 25.39, 25.48, 25.53, 28.88, 29.84, 30.31, 30.76, 48.39, 51.27, 51.91, 53.11, 53.26, 56.10, 59.53, 120.00, 126.64, 128.02, 128.71, 132.23, 134.30, 137.68, 142.96, 150.56, 150.83, 154.89, 158.37, 160.07; ES-MS m/z 497 (M+H). Anal. Calcd. for C₃₀H₃₆N₆O.0.2H₂O: C, 72.03; H, 7.33; N, 16.80. Found: C, 71.70; H, 7.39; N, 16.48.

### EXAMPLE 3

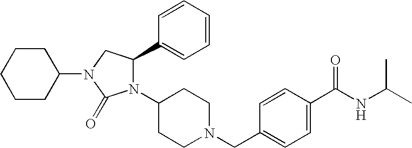

### COMPOUND 3: 4-[4-((R)-3-Cyclohexyl-2-oxo-5-phenyl-imidazolidin-1-yl)-piperidin-1-ylmethyl]-N-isopropyl-benzamide

Using general procedure E, 4-carboxybenzaldehyde (250 mg, 1.67 mmol) and isopropylamine (142 μL, 1.67 mmol) afforded 4-formyl-N-isopropyl-benzamide (300 mg, 94%).

COMPOUND 3 was isolated as a white solid (21 mg, 45%). ¹H NMR (CDCl₃) δ 0.91-2.11 (m, 16H), 1.24 (d, 6H, J=6.6 Hz), 2.67 (d, 1H, J=10.5 Hz), 2.85 (d, 1H, J=8.7 Hz), 3.04 (dd, 1H, J=8.7, 6.9 Hz), 3.45 (s, 2H), 3.59-3.70 (m, 1H), 3.63 (t, 1H, J=9.2 Hz), 3.76 (tt, 1H, J=11.4, 3.5 Hz), 4.27 (hex, 1H, J=6.2 Hz), 4.56 (dd, 1H, J=9.1, 6.4 Hz), 5.91 (d, 1H, J=7.3 Hz), 7.27-7.37 (m, 7H), 7.65 (d, 2H, J=8.5 Hz); ES-MS m/z 503 (M+1).

### EXAMPLE 4

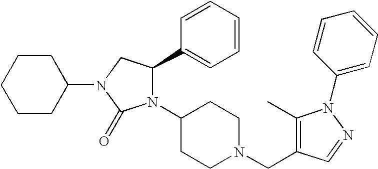

### COMPOUND 4: (R)-1-Cyclohexyl-3-[1-(5-methyl-1-phenyl-1H-pyrazol-4-ylmethyl)-piperidin-4-yl]-4-phenyl-imidazolidin-2-one

A mixture of ethyl acetoacetate (2.5 mL, 19.75 mmol) and DMF-dimethylacetal (3.15 mL, 23.69 mmol) was refluxed for 1.5 h. The excess of acetal was removed in vacuo. The residual material was purified by distillation (Kugelrohr; 200° C., 2 mm of Hg) to afford the intermediate (3.3 g, 90%) as a colorless oil.

To a solution of the above ester (500 mg, 2.69 mmol) in EtOH (6 mL) was added a solution of phenylhydrazine (280 μL, 2.82 mmol) in EtOH (6 mL). The solution was refluxed for 2 h. Standard aqueous work-up afforded the crude material that was used in the following step.

To a suspension of LAH (33 mg, 0.87 mmol) in THF (5 mL) at 0° C. was added a solution of the above ester (200 mg, 0.87 mmol) in THF (1 mL). The reaction mixture was stirred for 1 h and then treated with water (35 μL), NaOH [aq, 15%] (35 μL) and water (100 μL). The mixture was stirred for 15 min and the precipitate was filtered off. The solution was dried (Na₂SO₄), filtered and concentrated under reduced pressure. The crude material was purified by flash column chromatography on silica gel (30% ether in CH₂Cl₂ then 5% MeOH in CH₂Cl₂) to afford the alcohol (75 mg, 46%).

To a solution of the above alcohol (75 mg, 0.4 mmol) and 4-methylmorpholine-N-oxide (56 mg, 0.48 mmol) in CH₂Cl₂ (3 mL) was added TPAP (7 mg, 0.02 mmol). The mixture was stirred at rt for 1 h and the solution was filtered through a silica pad (30% ether in CH₂Cl₂) to afford 5-methyl-1-phenyl-1H-pyrazole-4-carbaldehyde (57 mg, 77%).

COMPOUND 4 was isolated as a white solid (45 mg, 74%). ¹H NMR (CDCl₃) δ 0.99-2.03 (m, 16H), 2.22 (s, 3H), 2.75 (d, 1H, J=11.0 Hz), 2.94 (d, 1H, J=10.2 Hz), 3.03 (dd, 1H, J=8.6, 6.9 Hz), 3.32 (s, 2H), 3.61-3.84 (m, 2H), 3.63 (t, 1H, J=9.2 Hz), 4.56 (dd, 1H, J=8.6, 6.9 Hz), 7.23-7.52 (m, 11H); ¹³C NMR (CDCl₃) δ 11.3, 25.9, 26.0, 30.1, 30.3, 30.8, 31.3, 48.9, 51.7, 52.4, 53.1, 53.3, 53.8, 56.4, 116.3, 125.3, 127.1, 127.9, 128.4, 129.1, 129.4, 137.6, 140.4, 141.5, 143.6, 160.6; ES-MS m/z 498 (M+1). Anal. Calcd. for C₃₁H₃₉N₅O.0.5CH₂Cl₂: C, 70.04; H, 7.46; N, 12.97. Found: C, 70.29; H, 7.55; N, 12.85.

### EXAMPLE 5

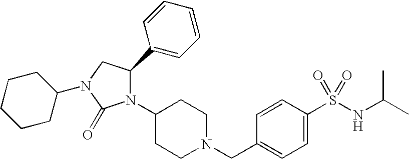

### COMPOUND 5: 4-[4-((R)-3-Cyclohexyl-2-oxo-5-phenyl-imidazolidin-1-yl)-piperidin-1-ylmethyl]-N-isopropyl-benzenesulfonamide

To a solution of p-TsCl (514 mg, 2.70 mmol) in THF (8.0 mL) was added a solution of methylamine (2.0M in THF, 4.0 mL, 8.0 mmol) and the resulting suspension was stirred at room temperature for 2 hours. Standard work-up and purification gave the sulfonamide as a white solid (409 mg, 82%).

A mixture of the sulfonamide (205 mg, 1.11 mmol), NBS (255 mg, 1.43 mmol) and Vazo®-88 (30 mg, 0.12 mmol) in CCl₄ (4.5 mL) was stirred at reflux under nitrogen for 3 hours. Standard work-up and purification by flash column chromatography on silica (CH₂Cl₂/Et₂O, 29:1) gave an approximate 7:2:1 mixture of the desired bromide, the dibromomethyl adduct and starting material (167 mg, ˜55%). Data for the bromomethyl sulfonamide: ¹H NMR (CDCl₃) δ 2.69 (d, 3H, J=5.2 Hz), 4.39 (br s, 1H), 4.50 (s, 2H), 7.55 (d, 2H, J=8.3 Hz), 7.84 (d, 2H, J=8.3 Hz).

COMPOUND 5 was isolated as a white solid (48.4 mg, 74%). ¹H NMR (CDCl₃) δ 0.94-1.10 (m, 1H), 1.07 (d, 6H, J=6.7 Hz), 1.18-1.45 (m, 6H), 1.58-1.69 (m, 2H), 1.71-1.82 (m, 4H), 1.86-2.07 (m, 3H), 2.61-2.70 (m, 1H), 2.78-2.86 (m, 1H), 3.05 (dd, 1H, J=8.4, 6.8 Hz), 3.40-3.51 (m, 1H), 3.45 (s, 2H), 3.58-3.69 (m, 1H), 3.64 (t, 1H, J=9.4 Hz), 3.77 (tt, 1H, J=11.6, 3.3 Hz), 4.23 (d, 1H, J=6.4 Hz), 4.57 (dd, 1H, J=9.4, 6.9 Hz), 7.28-7.39 (m, 5H), 7.38 (d, 2H, J=8.1 Hz), 7.76 (d, 2H, J=8.1 Hz); ¹³C NMR (CDCl₃) δ 23.70, 25.41, 25.50, 25.55, 28.94, 29.87, 30.31, 30.72, 45.98, 48.42, 51.29, 51.98, 53.13, 53.36, 56.08, 62.02, 126.64, 126.82, 128.01, 128.72, 129.19, 139.50, 143.02, 143.81, 160.13; ES-MS m/z 539 (M+1). Anal. Calcd. for C₃₀H₄₂N₄O₃S.0.2CH₂Cl₂: C, 65.27; H, 7.69; N, 10.08. Found: C, 65.02; H, 7.65; N, 9.81.

### EXAMPLE 6

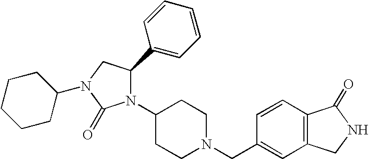

### COMPOUND 6: 5-[4-((R)-3-Cyclohexyl-2-oxo-5-phenyl-imidazolidin-1-yl)-piperidin-1-ylmethyl]-2,3-dihydro-isoindol-1-one

To a suspension of dimethyl aminoterephthalate (2.31 g, 11.0 mmol) in H₂O (14 mL) was added concentrated HCl (2.8 mL), and the mixture was cooled to 0° C. A solution of NaNO₂ (838 mg, 12.1 mmol) in H₂O (1.8 ml) was added dropwise then stirred at 0° C. for 10 minutes and neutralized with K₂CO₃(s). The mixture was added to CuCN (1.19 g, 13.3 mmol) and NaCN (1.30 g, 26.5 mmol) in H₂O (4.0 mL) at 60° C. then heated to 110° C. for 30 minutes to give 2-cyano-terephthalic acid dimethyl ester as a yellow solid (1.21 g, 50%) after work-up and purification.

A suspension of the nitrile from above (1.21 g, 5.52 mmol) and Raney® nickel (1 g) in MeOH (30 mL) was shaken under H₂ atmosphere (45 psi) for 19 h to give 1-oxo-2,3-dihydro-1H-isoindole-5-carboxylic acid methyl ester as a yellow solid (296 mg, 28%) after filtration and purification.

To a suspension of the ester from above (96 mg, 0.50 mmol) in CH₂Cl₂ (2.5 mL) was added DIBAL (1.0 M in CH₂Cl₂, 2.0 mL, 2.0 mmol) and the mixture stirred at room temperature for 30 minutes to give 5-hydroxymethyl-2,3-dihydro-isoindol-1-one as a brown solid (72 mg, 88%) after acidic work-up.

To a solution of the alcohol from above (72 mg, 0.44 mmol) in 10% MeOH/CH₂Cl₂ (5 mL) was added 90% MnO₂ (1.08 g, 11.2 mmol) and the mixture stirred at room temperature for 20 h to give 1-oxo-2,3-dihydro-1H-isoindole-5-carbaldehyde as a yellow solid (68 mg, 96%) after filtration.

COMPOUND 6 was isolated as a yellow foam (33 mg, 17%). ¹H NMR (CDCl₃) δ 1.02 (m, 1H), 1.17-1.44 (m, 6H), 1.62-1.76 (m, 6H), 1.88-2.07 (m, 3H), 2.69 (m, 1H), 2.86 (m, 1H), 3.04 (dd, 1H, J=8.7, 6.9 Hz), 3.49 (s, 2H), 3.62 (m, 2H), 3.77 (m, 1H), 4.40 (s, 2H), 4.57 (dd, 1H, J=9.3, 6.9 Hz), 6.55 (s, 1H), 7.33 (m, 7H), 7.76 (d, 1H, J=7.8 Hz); ¹³C NMR (CDCl₃) δ 25.86, 25.96, 26.00, 29.43, 30.32, 30.77, 31.11, 45.92, 48.88, 51.74, 52.53, 53.65, 53.83, 56.60, 63.06, 123.79, 123.84, 127.07, 128.43, 129.15, 131.35, 143.48, 143.56, 144.28, 160.59, 172.18; ES-MS m/z 473 (M+1). Anal. Calcd. for C₂₉H₃₆N₄O₂.0.2CH₂Cl₂: C, 71.63; H, 7.49; N, 11.44. Found: C, 71.34; H, 7.57; N, 11.19.

### EXAMPLE 7

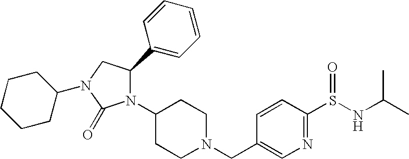

### COMPOUND 7: 5-[4-((R)-3-Cyclohexyl-2-oxo-5-phenyl-imidazolidin-1-yl)-piperidin-1-ylmethyl]-pyridine-2-carboxylic acid isopropylamide

Using general procedure F, 5-bromopicolic acid (550 mg, 0.2.72 mmol) and iso-propylamine (460 μL, 5.44 mmol) afforded 5-bromopyridine-2-carboxylic acid iso-propylamide (548 mg, 83%).

The above amide (178 mg, 0.783 mmol) was dissolved in dry THF (2.2 mL) and the solution was cooled to −78° C. Methyl lithium in a lithium bromide complex (780 μL, 1.5 M solution in diethyl ether, 1.17 mmol) was added and the solution was stirred for 5 minutes at −78° C. sec-Butyllithium (840 μL, 1.4 M solution in cyclohexane, 1.17 mmol) was added dropwise and the mixture was stirred another 5 minutes at −78° C. Finally, dry DMF (110 mL, 1.41 mmol) was added. The mixture was stirred for 5 minutes at −78° C. before the cooling bath was removed. The reaction was then stirred for 1 h at room temperature. Aqueous work-up and purification by flash column chromatography on silica gel (5% Et₂O/CH₂Cl₂) afforded 5-formylpyridine-2-carboxylic acid iso-propylamide (25 mg, 17%) as a yellow oil.

COMPOUND 7 was isolated as a white solid (21 mg, 32%). ¹H NMR (CDCl₃) δ 0.93-2.10 (m, 21H), 2.64 (d, 1H, J=11.8 Hz), 2.81 (d, 1H, J=10.5 Hz), 3.05 (t, 1H, J=8.4 Hz), 3.45 (s, 2H), 3.62 (t, 2H, J=8.9 Hz), 3.65-3.84 (m, 2H), 4.19-4.33 (m, 1H), 4.56 (dd, 1H, J=9.3, 6.8 Hz), 7.29-7.39 (m, 5H), 7.71 (dd, 1H, J=8.0, 2.1 Hz), 7.81 (d, 1H, J=8.7 Hz), 8.09 (d, 1H, J=7.8 Hz), 8.38 (br s, 1H); ¹³C NMR (CDCl₃) δ 22.8, 25.5, 25.6, 28.9, 29.9, 30.39, 30.9, 41.3, 46.1, 48.5, 51.4, 52.0, 53.2, 56.1, 59.7, 121.8, 126.7, 128.1, 128.8, 137.0, 137.7, 143.1, 148.4, 149.1; ES-MS m/z 526 (M+1). Anal. Calcd. for C₃₀H₄₁N₅O₂.0.46CH₄O: C, 70.58; H, 8.33; N, 13.51. Found: C, 70.70; H, 8.44; N, 13.25.

### EXAMPLE 8

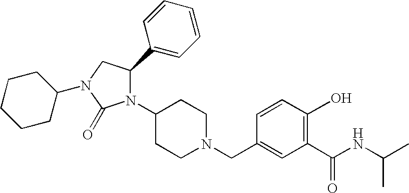

### COMPOUND 8: 5-[4-((R)-3-Cyclohexyl-2-oxo-5-phenyl-imidazolidin-1-yl)-piperidin-1-ylmethyl]-2-hydroxy-N-isopropyl-benzamide

COMPOUND 8 was isolated as a white solid (48 mg, 60%). ¹H NMR (CDCl₃) δ 1.05 (m, 1H), 1.28 (d, 6H, J=6.6 Hz), 1.23-1.46 (m, 6H), 1.65 (br m, 2H), 1.67-1.98 (br m, 9H), 2.70 (br d, 1H), 2.85 (br d, 1H), 3.03 (m, 1H), 3.30 (s, 2H), 3.55 (br m, 1H), 3.62 (t, 1H, J=9.0 Hz), 3.73 (m, 1H), 4.25 (m, 1H), 4.56 (m, 1H), 6.37 (br d, 1H, J=7.2 Hz), 6.85 (d, 1H, J=8.4 Hz), 7.18 (dd, 1H, J=8.4, 1.8 Hz), 7.33 (m, 5H); ES-MS m/z 519 (M+1).

### EXAMPLE 9

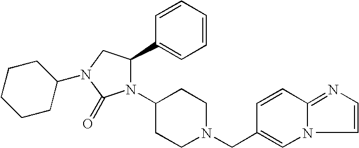

### COMPOUND 9: (R)-1-Cyclohexyl-3-(1-imidazo[1,2-a]pyridin-6-ylmethyl-piperidin-4-yl)-4-phenyl-imidazolidin-2-one

A solution of 2-bromoacetaldehyde diethylacetal (2.52 mL, 16.76 mmol) and HCl (12N, aq) (850 μL, 10.1 mmol) in water (10 mL) was heated at 90° C. for 2 h. The reaction mixture was cooled to room temperature and methanol (5 mL), 2-amino-5-bromopyridine (1.0 g, 5.78 mmol) and NaHCO₃ (1.36 g, 16.18 mmol) were added. The resulting mixture was stirred at 60° C. for and additional 30 min. The volatiles were removed in vacuo and the aqueous residue was extracted with CH₂Cl₂. The organic layer was dried (Na₂SO₄), filtered and concentrated. The crude material was purified by flash chromatography on silica gel (5% MeOH in CH₂Cl₂) to afford 6-bromoimidazo[1,2-a]pyridine (741 mg, 65%).

COMPOUND 9 was isolated as a white solid (27 mg, 39%). ¹H NMR (CDCl₃) δ 0.90-2.08 (m, 26H), 2.72 (d, 1H, J=11.0 Hz), 2.87 (d, 1H, J=9.1 Hz), 3.04 (t, 1H, J=7.6 Hz), 3.36 (s, 2H), 3.54-3.66 (m, 1H), 3.63 (t, 1H, J=9.3 Hz), 3.69-3.84 (m, 1H), 4.56 (dd, 1H, J=9.2, 7.0 Hz), 7.09 (d, 1H, J=9.1 Hz), 7.27-7.39 (m, 5H), 7.45-7.62 (m, 3H), 7.98 (s, 1H); ES-MS m/z 480 (M+Na).

### EXAMPLE 10

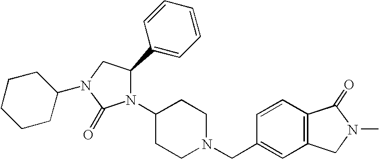

### COMPOUND 10: 5-[4-((R)-3-Cyclohexyl-2-oxo-5-phenyl-imidazolidin-1-yl)-piperidin-1-ylmethyl]-2-methyl-2.3-dihydro-isoindol-1-one

To a suspension of NaH (60% in mineral oil, 23 mg, 0.58 mmol) in DMF (0.4 mL) at 0° C. was added a suspension of 1-oxo-2,3-dihydro-1H-isoindole-5-carboxylic acid methyl ester (93 mg, 0.49 mmol) in DMF (2.0 mL). The mixture was stirred at 0° C. for 10 minutes then iodomethane (0.15 mL, 2.4 mmol) was added. The mixture was stirred at 0° C. for 1 h then concentrated in vacuo to give 2-methyl-1-oxo-2,3-dihydro-1H-isoindole-5-carboxylic acid methyl ester as yellow crystals (76 mg, 76%) after purification.

To a solution of the ester from above (76 mg, 0.37 mmol) in CH₂Cl₂ (1.9 mL) at 0° C. was added DIBAL (1.0 M in CH₂Cl₂, 2.2 mL, 2.2 mmol) then stirred at room temperature for 30 minutes to give 5-hydroxymethyl-2-methyl-2,3-dihydro-isoindol-1-one as yellow crystals (8 mg, 12%) after aqueous work-up and purification.

To a solution of the alcohol from above (28 mg, 0.16 mmol) and DIPEA (0.030 mL, 0.17 mmol) in CH₂Cl₂ (1.6 mL) at 0° C. was added MsCl (0.012 mL, 0.16 mmol). The solution was stirred at 0° C. for 20 minutes then concentrated in vacuo.

Using general procedure G, the residue from above and (R)-1-cyclohexyl-4-phenyl-3-piperidin-4-yl-imidazolidin-2-one (63 mg, 0.19 mmol) afforded COMPOUND 10 as a yellow foam (13 mg, 17%). ¹H NMR (CDCl₃) δ 1.02 (m, 1H), 1.17-1.44 (m, 6H), 1.61-2.05 (m, 9H), 2.69 (m, 1H), 2.85 (m, 1H), 3.04 (dd, 1H, J=8.6, 7.1 Hz), 3.18 (s, 3H), 3.47 (s, 2H), 3.62 (m, 2H), 3.77 (m, 1H), 4.32 (s, 2H), 4.57 (dd, 1H, J=9.3, 6.6 Hz), 7.31 (m, 7H), 7.72 (d, 1H, J=7.5 Hz); ES-MS m/z 487 (M+H).

### EXAMPLE 11

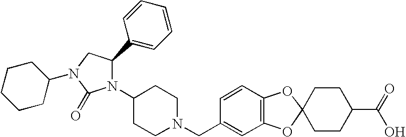

### COMPOUND 11: 5-[4-((R)-3-Cyclohexyl-2-oxo-5-phenyl-imidazolidin-1-yl)-piperidin-1-ylmethyl]-spiro[1,3-benzodioxole-2,1′-cyclohexane]-4′-carboxylic acid

A solution of ethyl 4-cyclohexanonecarboxylate (631 mg, 3.71 mmol), 4-methyl-catechol (576 mg, 4.64 mmol) and p-TSA.H₂O (71 mg, 0.37 mmol) in CH₂Cl₂ (20 mL) was stirred at reflux for 17 hours. Standard work-up followed by purification by flash column chromatography on silica (hexane/EtOAc, 9:1) gave 5-methyl-spiro[1,3-benzodioxole-2,1′-cyclohexane]-4′-carboxylic acid ethyl ester as a colourless liquid (679 mg, 66%). ¹H NMR (CDCl₃) δ 1.27 (t, 3H, J=7.2 Hz), 1.74-1.84 (m, 2H), 1.89-2.08 (m, 4H), 2.09-2.17 (m, 2H), 2.26 (s, 3H), 2.38-2.48 (m, 1H), 4.16 (q, 2H, J=7.2 Hz), 6.54-6.64 (m, 3H).

A mixture of the benzodioxolane (289 mg, 1.05 mmol), NBS (238 mg, 1.34 mmol) and Vazo®-88 (34 mg, 0.14 mmol) in CCl₄ (5 mL) was stirred at reflux under nitrogen for 70 minutes. Standard work-up and purification gave an approximately 7:1 mixture of 5-(bromomethyl)-spiro[1,3-benzodioxole-2,1′-cyclohexane]-4′-carboxylic acid ethyl ester and the α,α-dibromo species, contaminated with trace amounts of the radical initiator (395 mg).

Following general procedure G: a solution of the impure bromide (˜85%, 132 mg, 031 mmol), (R)-1-cyclohexyl-4-phenyl-3-piperidin-4-yl-imidazolidin-2-one (115 mg, 0.35 mmol), BHT (11 mg, 0.05 mmol) and DIPEA (0.10 mL, 0.57 mmol) in CH₃CN (2.0 mL) was stirred at 55° C. for 18.5 hours. Standard work-up and purification gave a diastereomeric mixture of 5-[4-((R)-3-cyclohexyl-2-oxo-5-phenyl-imidazolidin-1-yl)-piperidin-1-ylmethyl]-spiro[1,3-benzodioxole-2,1′-cyclohexane]-4′-carboxylic acid ethyl ester as a white foam (104 mg, 56%).

Following general procedure H, the ethyl ester (86 mg, 0.14 mmol) and 10M NaOH (0.30 mL, 3.0 mmol) in MeOH (3.0 mL) was stirred at 60° C. for 2.5 hours giving COMPOUND 11 as a light yellow solid (80.9 mg, 99%). ¹H NMR (MeOH-d₄) δ 1.03-1.18 (m, 1H), 1.26-1.48 (m, 5H), 1.55-1.93 (m, 1H), 1.96-2.11 (m, 4H), 2.16-2.39 (m, 2H), 2.48-2.69 (m, 2H), 3.06-3.27 (m, 2H), 3.11 (dd, 1H, J=8.4, 7.5 Hz), 3.53-3.91 (m, 5H), 4.70 (dd, 1H, J=8.7, 7.5 Hz), 6.69-6.83 (m, 3H), 7.29-7.41 (m, 5H); ¹³C NMR (MeOH-d₄) δ 26.48, 26.65, 26.75, 27.27, 27.63 and 28.02, 29.14 and 29.19, 30.87, 31.23, 34.92, 35.07, 43.73, 49.48, 51.52 and 51.73, 52.26 and 52.65, 52.87 and 52.98, 53.02, 57.63 and 57.84, 61.25 and 61.52, 108.95 and 109.28, 111.50 and 111.66, 119.98 and 120.17, 125.15 and 125.36, 125.48 and 125.65, 127.99, 129.48 and 129.50, 130.08 and 130.10, 143.47 and 143.54, 149.18 and 149.22, 149.44 and 149.72, 161.71, 181.04 and 181.46; ES-MS m/z 574 (M+H).

### EXAMPLE 12

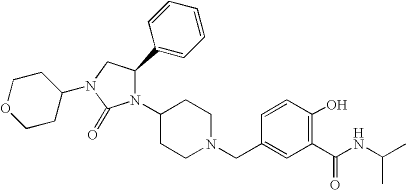

### COMPOUND 12: 2-Hydroxy-N-isopropyl-5-{4-[(R)-2-oxo-5-phenyl-3-(tetrahydro-pyran-4-yl)-imidazolidin-1-yl]-piperidin-1-ylmethyl}-benzamide

Following General Procedure E: a mixture of 5-formylsalicylic acid (0.332 g 2.00 mmol), isopropylamine (0.124 g, 2.10 mmol), DIPEA (0.361 g, 2.80 mmol), EDCI (0.481 g, 2.50 mmol), HOBT (0.338 g, 2.50 mmol) in DMF (4 mL) was stirred at room temperature for 2 days. Standard work-up and purification afforded 5-formyl-2-hydroxy-N-isopropyl-benzamide as an off-white solid (0.180 g, 43%). ¹H NMR (CDCl₃) δ 1.32 (d, 6H, J=6.31 Hz), 4.31 (septet, 1H, J=6.3 Hz), 6.36 (br s, 1H), 7.09 (d, 1H, J=8.7 Hz), 7.87 (dd, 1H, J=8.4, 1.5 Hz), 7.99 (d, 1H, J=1.5 Hz), 9.87 (s, 1H).

Following General Procedure A: to a solution of (R)-4-phenyl-3-piperidin-4-yl-1-(tetrahydro-pyran-4-yl)-imidazolidin-2-one (0.066 g, 0.20 mmol) and the above aldehyde (0.063 g, 0.30 mmol) in CH₂Cl₂ (3 mL) was added NaBH(OAc)₃ (0.074 mg, 0.35 mmol) and the mixture stirred at room temperature overnight. Standard work-up and purification gave COMPOUND 12 as a white foam (0.070 g, 67%). ¹H NMR (CDCl₃) δ 1.28 (d, 6H, J=6.3 Hz), 1.42-1.48 (m, 1H), 1.60-1.72 (m, 5H), 1.82-2.05 (m, 4H), 2.68-2.73 (m, 1H), 2.83-2.88 (m, 1H), 3.05 (dd, 1H, J=8.4, 6.9 Hz), 3.31 (s, 2H), 3.44-3.50 (m, 2H), 3.50-3.67 (m, 2H), 3.96-4.05 (m, 3H), 4.26 (septet, 1H, J=6.3 Hz), 4.59 (dd, 1H, J=9.0, 6.9 Hz), 6.27 (br s, 1H), 6.85 (d, 1H, J=8.4 Hz), 7.18-7.23 (m, 2H), 7.28-7.35 (m, 5H); ¹³C NMR (CDCl₃) δ 22.83, 29.03, 29.96, 30.44, 41.96, 48.58, 48.89, 52.32, 53.28, 56.72, 62.36, 67.38, 67.49, 114.55, 118.14, 126.22, 126.96, 128.51, 129.08, 135.10, 142.61, 160.24, 160.80, 169.34; ES-MS m/z 521 (M+1). Anal. Calcd. for C₃₀H₄₀N₄O₄.0.5CH₂Cl₂: C, 65.05; H, 7.34; N, 9.95. Found: C, 64.90; H, 7.42; N, 9.91.

### EXAMPLE 13

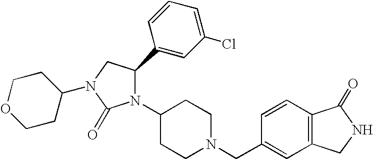

### COMPOUND 13: 5-{4-[5-(3-Chloro-phenyl)-2-oxo-3-(tetrahydro-pyran-4-yl)-imidazolidin-1-yl]-piperidin-1-ylmethyl}-2,3-dihydro-isoindol-1-one

Following general procedure A, 1-oxo-2,3-dihydro-1H-isoindole-5-carbaldehyde (see EXAMPLE 6) (50.0 mg, 0.311 mmol) was reacted with 4-(3-chlorophenyl)-3-piperidin-4-yl-1-(tetrahydro-pyran-4-yl)-imidazolidin-2-one (113 mg, 0.311 mmol) to provide COMPOUND 13 as a white foam (39.0 mg, 56%). ¹H NMR (CDCl₃) δ 1.19-1.31 (m, 1H), 1.43 (d, 1H, J=12.0 Hz), 1.53-1.72 (m, 4H), 1.83-2.07 (m, 4H), 2.71 (d, 1H, J=11.1 Hz), 2.86 (d, 1H, J=10.5 Hz), 3.01 (dd, 1H, J=8.7, 6.6 Hz), 3.42-3.49 (m, 2H), 3.49 (s, 2H), 3.59-3.66 (m, 1H), 3.65 (t, 1H, J=9.0 Hz), 3.96-4.08 (m, 3H), 4.40 (s, 2H), 4.56 (dd, 1H, J=9.0, 6.6 Hz), 7.17-7.38 (m, 7H), 7.76 (d, 1H, J=7.8 Hz); ¹³C NMR (CDCl₃) δ 29.51, 30.30, 30.44, 31.27, 45.98, 48.66, 49.11, 52.55, 53.48, 53.72, 55.92, 63.03, 67.54, 67.60, 123.78, 123.87, 125.06, 127.07, 128.80, 129.21, 130.62, 131.49, 135.11, 143.33, 144.33, 145.38, 160.28, 172.27; ES-MS m/z 509 (M+H). Anal. Calcd. for C₂₈H₃₃ClN₄O₃.0.7CH₂Cl₂: C, 60.64; H, 6.10; N, 9.86. Found: C, 60.56; H, 6.03; N, 9.77.

Examples 14 to 21 were prepared following the scheme illustrated below. RNH₂ is as defined in the table.

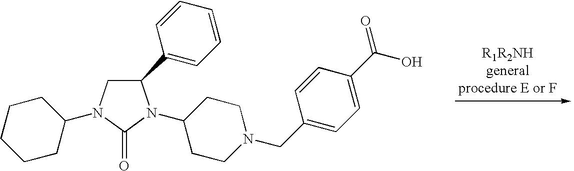

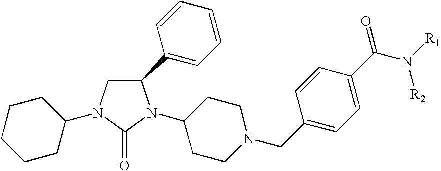

| Example | R₁R₂NH |
|---|---|
| 14 | cyclopentylamine |
| 15 | 2-methoxy-ethylamine |
| 16 | 2,2,2-trifluoro-ethylamine |
| 17 | 4-amino-cyclohexanol hydrochloride |
| 18 | 4-methoxybenzylamine |
| 19 | (±)-alanine methyl ester hydrochloride |
| 20 | tetrahydropyran-4-ylamine (Renhowe, Paul A, U.S. Pat. No. 2002/137939 A1) |
| 21 | 4-hydroxypiperidine |

### EXAMPLE 14

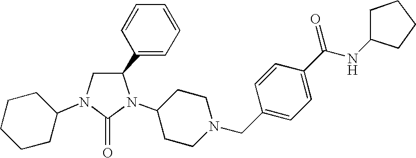

### COMPOUND 14: 4-[4-((R)-3-Cyclohexyl-2-oxo-5-phenyl-imidazolidin-1-yl)-piperidin-1-ylmethyl]-N-cyclopentyl-benzamide

COMPOUND 14 was isolated as a colourless foam (42 mg, 57%). ¹H NMR (CDCl₃) δ 0.91-2.09 (m, 25H), 2.65 (m, 1H), 2.82 (m, 1H), 3.04 (dd, 1H, J=7.7, 1.5 Hz), 3.42 (s, 2H), 3.63 (m, 2H), 3.77 (m, 1H), 4.38 (m, 1H), 4.57 (dd, 1H, J=6.9, 2.6 Hz), 6.00 (d, 1H, J=7.3 Hz), 7.32 (m, 7H), 7.64 (d, 2H, J=8.5 Hz).

### EXAMPLE 15

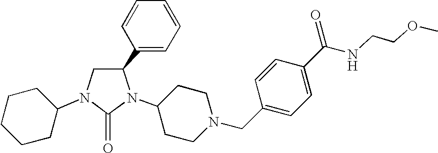

### COMPOUND 15: 4-[4-((R)-3-Cyclohexyl-2-oxo-5-phenyl-imidazolidin-1-yl)-piperidin-1-ylmethyl]-N-(2-methoxy-ethyl)-benzamide

COMPOUND 15 was isolated as a colourless foam (22 mg, 30%). ¹H NMR (CDCl₃) δ 0.99-2.00 (m, 16H), 2.67 (m, 1H), 2.83 (m, 1H), 3.04 (m, 1H), 3.38 (s, 3H), 3.43 (s, 2H), 3.55 (m, 2H), 3.62 (m, 4H), 3.77 (m, 1H), 4.57 (dd, 1H, J=7.4, 1.7 Hz), 6.46 (m, 1H), 7.30 (m, 7H), 7.68 (d, 2H, J=8.4 Hz).

### EXAMPLE 16

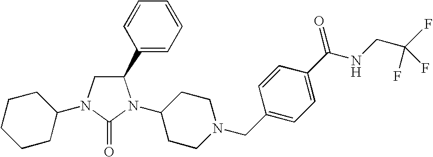

### COMPOUND 16: 4-[4-((R)-3-Cyclohexyl-2-oxo-5-phenyl-imidazolidin-1-yl)-piperidin-1-ylmethyl]-N-(2,2,2-trifluoro-ethyl)-benzamide

COMPOUND 16 was isolated as a colourless foam (12 mg, 16%). ¹H NMR (CDCl₃) δ 0.85-2.03 (m, 17H), 2.64 (m, 1H), 2.82 (m, 1H), 3.04 (dd, 1H, J=8.0, 1.2 Hz), 3.43 (s, 2H), 3.63 (m, 2H), 3.76 (m, 1H), 4.12 (m, 2H), 4.57 (dd, 1H, J=7.0, 2.2 Hz), 6.50 (t, 1H, J=6.3 Hz), 7.30 (m, 7H), 7.71 (d, 2H, J=8.2 Hz).

### EXAMPLE 17

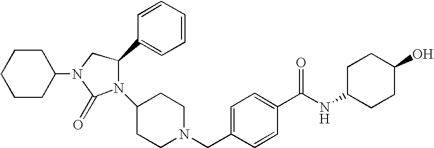

### COMPOUND 17: 4-[4-((R)-3-Cyclohexyl-2-oxo-5-phenylimidazolidin-1-yl)-piperidin-1-ylmethyl]-N-(4-hydroxycyclohexyl)-benzamide

COMPOUND 17 was isolated as a white solid (108 mg, 60%). ¹H NMR (CDCl₃) δ 0.85-2.17 (m, 25H), 2.30-2.40 (m, 2H), 2.63 (d, 1H, J=11.6 Hz), 2.81 (d, 11H, J=8.4 Hz), 3.02 (t, 1H, J=7.1. Hz), 3.41 (s, 2H), 3.55-4.01 (m, 3H), 4.55 (dd, 1H, J=9.2, 7.0 Hz), 6.13 (d, 11H, J=7.8 Hz), 7.27 (d, 2H, J=7.7 Hz), 7.28-7.30 (m, 5H), 7.64 (d, 2H, J=8.4 Hz); ES-MS m/z 559 (M+1).

### EXAMPLE 18

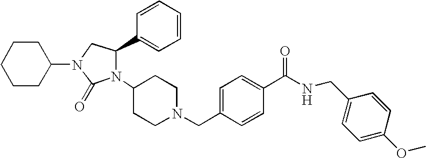

### COMPOUND 18: 4-[4-((R)-3-Cyclohexyl-2-oxo-5-phenyl-imidazolidin-1-yl)-piperidin-1-ylmethyl]-N-(4-methoxybenzyl)-benzamide

COMPOUND 18 was isolated as a white solid (13 mg, 21%). ¹H NMR (CDCl₃) δ 0.91-2.09 (m, 16H), 2.66 (d, 1H, J=11.0 Hz), 2.83 (d, 1H, J=9.6 Hz), 3.03 (t, 1H, J=8.3 Hz), 3.43 (s, 2H), 3.63 (t, 2H, J=9.2 Hz), 3.67-3.81 (m, 1H), 3.80 (s, 3H), 4.51-4.61 (m, 3H), 6.30 (t, 1H, J=4.7 Hz), 6.87 (d, 2H, J=8.2 Hz), 7.23-7.37 (m, 9H), 7.68 (d, 2H, J=8.0 Hz); ES-MS m/z 603 (M+Na).

### EXAMPLE 19

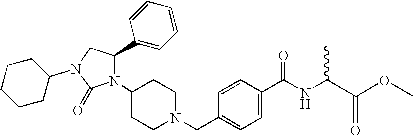

### COMPOUND 19: 2-{4-[4-((R)-3-Cyclohexyl-2-oxo-5-phenyl-imidazolidin-1-yl)-piperidin-1-ylmethyl]-benzoylamino}-propionic acid methyl ester

COMPOUND 19 was isolated as a white foam (52.9 mg, 70%). ¹H NMR (CDCl₃) δ 0.94-1.09 (m, 1H), 1.14-1.45 (m, 6H), 1.51 (s, 3H, J=7.1 Hz), 1.59-1.81 (m, 6H), 1.84-2.06 (m, 3H), 2.61-2.70 (m, 1H), 2.79-2.88 (m, 1H), 3.04 (dd, 1H, J=8.4, 6.9 Hz), 3.44 (s, 2H), 3.60-3.82 (m, 3H), 3.79 (s, 3H), 4.57 (dd, 1H, J=9.5, 6.9 Hz), 4.79 (dq, 1H, J=7.2, 7.1 Hz), 6.68 (d, 1H, J=6.5 Hz), 7.28-7.37 (m, 7H), 7.71 (d, 2H, J=8.4 Hz); ES-MS m/z 547 (M+H).

### EXAMPLE 20

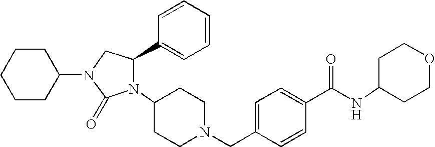

### COMPOUND 20: 4-[4-((R)-3-Cyclohexyl-2-oxo-5-phenylimidazolidin-1-yl)-piperidin-1-ylmethyl]-N-(tetrahydro-pyran-4-yl)-benzamide

COMPOUND 20 was isolated as a white solid (22 mg, 37%). ¹H NMR (CDCl₃) δ 0.92-2.10 (m, 19H), 2.66 (d, 1H, J=10.9 Hz), 2.84 (d, 1H, J=9.9 Hz), 3.04 (t, 1H, J=8.4 Hz), 3.37-3.82 (m, 8H), 3.99 (d, 2H, J=10.9 Hz), 4.11-4.29 (m, 1H), 4.56 (dd, 1H, J=9.2, 7.1 Hz), 5.99 (d, 1H, J=7.5 Hz), 7.27-7.38 (m, 7H), 7.66 (d, 2H, J=7.8 Hz); ES-MS m/z 545 (M+1).

### EXAMPLE 21

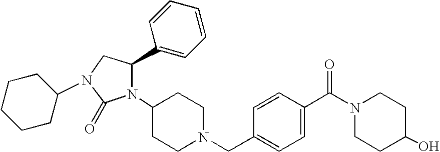

### COMPOUND 21: (R)-1-Cyclohexyl-3-{1-[4-(4-hydroxy-piperidine-1-carbonyl)-benzyl]-piperidin-4-yl}-4-phenyl-imidazolidin-2-one

COMPOUND 21 was isolated as a white foam (122 mg, 67%). ¹H NMR (CDCl₃) δ 0.94-1.09 (m, 1H), 1.16-1.45 (m, 6H), 1.47-2.04 (m, 14H), 2.63-2.72 (m, 1H), 2.82-2.90 (m, 1H), 3.03 (dd, 1H, J=8.6, 7.1 Hz), 3.13-3.47 (m, 2H), 3.41 (s, 2H), 3.58-3.81 (m, 4H), 3.91-4.01 (m, 1H), 4.19 (br s, 1H), 4.57 (dd, 1H, J=9.4, 6.9 Hz), 7.24-7.37 (m, 9H); ES-MS m/z 545 (M+H).

### EXAMPLE 22

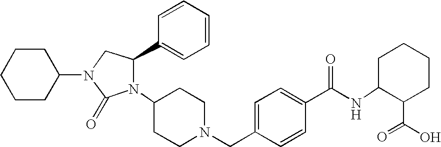

### COMPOUND 22: (trans)-2-{4-[4-((R)-3-Cyclohexyl-2-oxo-5-phenyl-imidazolidin-1-yl)-piperidin-1-ylmethyl]-benzoylamino}-cyclohexanecarboxylic acid

Using general procedure E, 4-[4-((R)-3-cyclohexyl-2-oxo-5-phenyl-imidazolidin-1-yl)-piperidin-1-ylmethyl]-benzoic acid (50 mg, 0.11 mmol) and (trans)-2-amino-cyclohexanecarboxylic acid ethyl ester (21 mg, 0.12 mmol) in DMF (2 mL) afforded (trans)-2-{4-[4-((R)-3-cyclohexyl-2-oxo-5-phenylimidazolidin-1-yl)-piperidin-1-ylmethyl]-benzoylamino}-cyclohexanecarboxylic acid ethyl ester as a white solid (54 mg, 80%).

Using general procedure H with the above ethyl ester (50 mg, 0.08 mmol) gave COMPOUND 22 (38 mg, 79%) as a white solid. ¹H NMR (CDCl₃) (mixture of diastereoisomers) δ 0.85-2.63 (m, 26H), 2.68-3.20 (m, 3H), 3.28-4.20 (m, 5H), 4.56-4.63 (m, 1H), 6.88-7.00 (m, 1H), 7.10-7.55 (m, 9H); ES-MS m/z 587 (M+1).

### EXAMPLE 23

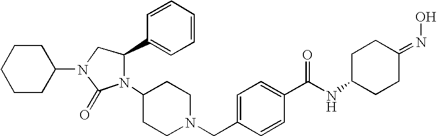

### COMPOUND 23: 4-[4-((R)-3-Cyclohexyl-2-oxo-5-phenylimidazolidin-1-yl)-piperidin-1-ylmethyl]-N-(4-hydroxyimino-cyclohexyl)-benzamide

To a solution of 4-[4-((R)-3-cyclohexyl-2-oxo-5-phenylimidazolidin-1-yl)-piperidin-1-ylmethyl]-N-(4-hydroxycyclohexyl)-benzamide (COMPOUND 17) in dichloromethane (5 mL) was added 4-methylmorpholine-N-oxide (24 mg, 0.21 mmol) and TPAP (6 mg, 0.02 mmol). The reaction was stirred at room temperature for 1 h. The solvent was removed in vacuo and the residue was purified on a silica gel pad (100% CH₂Cl₂ then 5% MeOH in CH₂Cl₂) to afford the corresponding ketone (80 mg, 80%) as a white solid.

A solution of 4-[4-((R)-3-cyclohexyl-2-oxo-5-phenylimidazolidin-1-yl)-piperidin-1-ylmethyl]-N-(4-oxocyclohexyl)-benzamide (74 mg, 0.13 mmol), hydroxylamine hydrochloride (92 mg, 69.49 mmol) and sodium acetate (213 mg, 82.03 mmol) in methanol (2 mL) was stirred at reflux for 18 h. The solvent was removed and the residue was purified by flash column chromatography on silica gel (5% MeOH in CH₂Cl₂, 2% NH₄OH) to give COMPOUND 23 (23 mg, 31%) as a white solid. ¹H NMR (CDCl₃) δ 0.92-2.51 (m, 25H), 2.68 (d, 1H, J=10.6 Hz), 2.87 (d, 1H, J=10.1 Hz), 3.04 (t, 1H, J=8.2 Hz), 3.28 (d, 1H, J=14.8 Hz), 3.46 (s, 2H), 3.63 (t, 1H, J=9.4 Hz), 3.67-3.84 (m, 1H), 4.14-4.29 (m, 1H), 4.57 (dd, 1H, J=9.1, 6.6 Hz), 6.09 (d, 1H, J=7.4 Hz), 7.27-7.40 (m, 7H), 7.67 (d, 2H, J=8.5 Hz); ES-MS m/z 572 (M+1).

### EXAMPLE 24

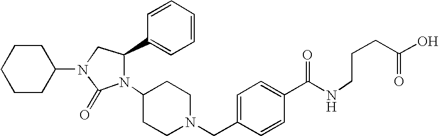

### COMPOUND 24: 4-{4-[4-((R)-3-Cyclohexyl-2-oxo-5-phenyl-imidazolidin-1-yl)-piperidin-1-ylmethyl]-benzoylamino}-butyric acid

Using general procedure E, 4-[4-((R)-3-cyclohexyl-2-oxo-5-phenylimidazolidin-1-yl)-piperidin-1-ylmethyl]-benzoic acid (50 mg, 0.11 mmol) and 4-aminobutyric acid ethyl ester hydrochloride (20 mg, 0.12 mmol) in DMF (2 mL) afforded 4-{4-[4-((R)-3-cyclohexyl-2-oxo-5-phenylimidazolidin-1-yl)-piperidin-1-ylmethyl]-benzoylamino}-butyric acid ethyl ester as a white solid (33 mg, 52%).

Using general procedure H, the above ethyl ester (28 mg, 0.05 mmol) gave COMPOUND 24 (22 mg, 81%) as a white solid. ¹H NMR (CD₃OD) δ 0.98-2.27 (m, 19H), 2.31 (t, 2H, J=7.6 Hz), 2.84 (d, 1H, J=10.9 Hz), 2.99 (d, 1H, J=11.6 Hz), 3.09 (dd, 1H, J=8.8, 7.1 Hz), 3.40 (t, 2H, J=6.7 Hz), 3.46-3.69 (m, 2H), 3.63 (s, 2H), 3.75 (t, 1H, J=9.2 Hz), 4.71 (dd, 1H, J=9.7, 7.0 Hz), 7.24-7.44 (m, 7H), 7.77 (d, 2H, J=7.7 Hz); ES-MS m/z 547 (M+1).

### EXAMPLE 25

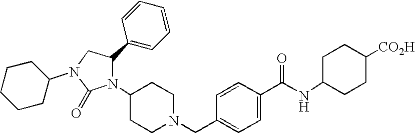

### COMPOUND 25: 4-{4-[4-((R)-3-Cyclohexyl-2-oxo-5-phenyl-imidazolidin-1-yl)-piperidin-1-ylmethyl]-benzoylamino}-cyclohexanecarboxylic acid

To a suspension of 4-aminocyclohexanecarboxylic acid (495 mg, 3.46 mmol) in CH₂Cl₂ (20 mL) was added thionyl chloride (0.75 mL, 10.3 mmol) and the resulting mixture was stirred at room temperature for 40 minutes. The solvent was removed and the residue was dried under reduced pressure. Methanol (20 mL) was added and the solution was stirred at reflux for 2.25 hours. Once cooled, the solvent was removed under reduced pressure, the residue was made basic with 0.5M NaOH (25 mL) and was extracted with CH₂Cl₂ (25 mL×3). The organic solution was dried (Na₂SO₄), filtered and concentrated under reduced pressure, giving crude methyl 4-aminocyclohexanecarboxylate as a yellow liquid (529 mg, 97%). ¹H NMR (CDCl₃) δ 1.02-2.07 (m, 8H), 1.21 (s, 2H), 2.17-2.27 and 2.43-2.51 (m, 1H), 2.60-2.69 and 2.79-2.88 (m, 1H), 3.65 and 3.67 (s, 3H).

Following general procedure E: a mixture of the 4-amino ester (29 mg, 0.18 mmol), 4-[4-((R)-3-cyclohexyl-2-oxo-5-phenyl-imidazolidin-1-yl)-piperidin-1-ylmethyl]-benzoic acid (65 mg, 0.14 mmol), EDCI (35 mg, 0.18 mmol), HOBT (32 mg, 0.24 mmol) and NMM (30 μL, 0.27 mmol) in DMF (1.0 mL) was stirred at room temperature for 16 hours. Standard work-up and purification gave a diastereomeric mixture of 4-{4-[4-((R)-3-cyclohexyl-2-oxo-5-phenyl-imidazolidin-1-yl)-piperidin-1-ylmethyl]-benzoylamino}-cyclohexanecarboxylic acid methyl ester as an off-white foam (69.1 mg, 82%).

Following general procedure H, a solution of the methyl ester (55 mg, 0.092 mmol) and 10M NaOH (0.20 mL, 2.0 mmol) in MeOH (1.8 mL) was stirred at 60° C. for 2.5 hours giving COMPOUND 25 as a pale yellow solid (59.6 mg, quantitative). ¹H NMR (MeOH-d₄) δ 1.03-1.19 (m, 1H), 1.214-1.58 (m, 8H), 1.60-1.85 (m, 1OH), 1.98-2.27 (m, 5H), 2.39-2.56 (m, 1H), 2.81-2.90 (m, 1H), 2.95-3.04 (m, 1H), 3.10 (dd, 1H, J=8.3, 7.0 Hz), 3.45-3.58 (m, 1H), 3.59-3.71 (m, 1H), 3.64 (s, 2H), 3.75 (t, 1H, J=9.1 Hz), 3.80-4.01 (m, 1H), 4.70 (dd, 1H, J=9.2, 7.0 Hz), 7.28-7.40 (m, 7H), 7.76 (d, 2H, J=8.2 Hz); ES-MS m/z 587 (M+H).

### EXAMPLE 26

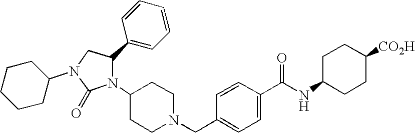

### COMPOUND 26: cis-4-{4-[4-((R)-3-Cyclohexyl-2-oxo-5-phenyl-imidazolidin-1-yl)-piperidin-1-ylmethyl]-benzoylamino}-cyclohexanecarboxylic acid

To a suspension of cis-4-aminocyclohexanecarboxylic acid (165 mg, 1.15 mmol) in CH₂Cl₂ (6 mL) was added thionyl chloride (0.25 mL, 3.4 mmol) and the resulting mixture was stirred at room temperature for 45 minutes. The solvent was removed and the residue was dried under reduced pressure. Methanol (6 mL) was added and the solution was stirred at reflux for 2 hours. Standard work-up gave the crude methyl cis-4-aminocyclohexanecarboxylate as a yellow liquid (121 mg, 67%). ¹H NMR (CDCl₃) δ 1.25 (s, 2H), 1.30-1.41 (m, 2H), 1.52-1.70 (m, 4H), 1.94-2.05 (m, 2H), 2.41-2.49 (m, 1H), 2.78-2.87 (m, 1H), 3.66 (s, 3H).

Following general procedure E: a mixture of the 4-amino ester (25 mg, 0.16 mmol), 4-[4-((R)-3-cyclohexyl-2-oxo-5-phenyl-imidazolidin-1-yl)-piperidin-1-ylmethyl]-benzoic acid (63 mg, 0.16 mmol), EDCI (34 mg, 0.18 mmol), HOBT (27 mg, 0.20 mmol) and NMM (30 μL, 0.27 mmol) in DMF (1.0 mL) was stirred at room temperature for 18 hours to give cis-4-{4-[4-((R)-3-cyclohexyl-2-oxo-5-phenyl-imidazolidin-1-yl)-piperidin-1-ylmethyl]-benzoylamino}-cyclohexanecarboxylic acid methyl ester as a white foam (74.8 mg, 92%).

Following general procedure H: a solution of the methyl ester (26.7 mg, 0.044 mmol) and 10M NaOH (0.10 mL, 1.0 mmol) in MeOH (0.90 mL) was stirred at 60° C. for 2 hours giving COMPOUND 26 as a white solid (21.3 mg, 82%). ¹H NMR (MeOH-d₄) δ 1.03-1.20 (m, 1H), 1.25-1.55 (m, 7H), 1.58-1.85 (m, 11H), 1.98-2.23 (m, 5H), 2.48-2.57 (m, 1H), 2.79-2.88 (m, 1H), 2.92-3.02 (m, 1H), 3.09 (dd, 1H, J=8.7, 7.2 Hz), 3.45-3.78 (m, 3H), 3.62 (s, 2H), 3.90-4.02 (m, 1H), 4.70 (dd, 1H, J=9.5, 7.2 Hz), 7.28-7.40 (m, 7H), 7.75 (d, 2H, J=7.9 Hz); ES-MS m/z 587 (M+H).

### EXAMPLE 27

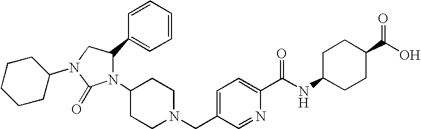

### COMPOUND 27: cis-4-({5-[4-((R)-3-Cyclohexyl-2-oxo-5-phenyl-imidazolidin-1-yl)-piperidin-1-ylmethyl]-pyridine-2-carbonyl}-amino)-cyclohexanecarboxylic acid

To a solution of 5-methylpyridine-2-carboxylic acid methyl ester (140 mg, 0.93 mmol) in CCl₄ (8 mL) was added NBS (181 mg, 1.02 mmol) and VAZO™ (23 mg, 0.09 mmol). The reaction was stirred at reflux for 18 h. Standard work-up and purification by flash chromatography on silica gel (10% ether in CH₂Cl₂) afforded 5-bromomethylpyridine-2-carboxylic acid methyl ester (107 mg, 50%).

Using general procedure G, (R)-1-cyclohexyl-4-phenyl-3-piperidin-4-ylimidazolidin-2-one (160 mg, 0.49 mmol) and the above bromide (107 mg, 0.47 mmol) afforded 5-[4-((R)-3-cyclohexyl-2-oxo-5-phenylimidazolidin-1-yl)-piperidin-1-ylmethyl]-pyridine-2-carboxylic acid methyl ester (175 mg, 78%).

Using general procedure H, the above methyl ester (175 mg, 0.37 mmol) gave 5-[4-((R)-3-cyclohexyl-2-oxo-5-phenylimidazolidin-1-yl)-piperidin-1-ylmethyl]-pyridine-2-carboxylic acid (137 mg, 80%) as a white solid.

Using general procedure A, 5-[4-((R)-3-cyclohexyl-2-oxo-5-phenylimidazolidin-1-yl)-piperidin-1-ylmethyl]-pyridine-2-carboxylic acid (45 mg, 0.10 mmol) and methyl (cis)-4-aminocyclohexanecarboxylate (see EXAMPLE 25) (15 mg, 0.10 mmol) gave (cis)-4-({5-[4-((R)-3-cyclohexyl-2-oxo-5-phenylimidazolidin-1-yl)-piperidin-1-ylmethyl]-pyridine-2-carbonyl}-amino)-cyclohexanecarboxylic acid methyl ester (43 mg, 71%).

Using general procedure H with the above methyl ester (36 mg, 0.06 mmol) gave COMPOUND 27 (30 mg, 86%) as a white solid. ¹H NMR (CD₃OD) δ 1.01-2.09 (m, 22H), 2.24-2.44 (m, 1H), 2.50-2.61 (m, 1H), 2.79-2.97 (m, 2H), 3.12 (dd, 1H, J=8.7, 7.1 Hz), 3.31-3.44 (m, 1H), 3.50-3.70 (m, 2H), 3.76 (t, 1H, J=9.2 Hz), 3.96-4.09 (m, 1H), 4.25 (s, 2H), 4.71 (dd, 1H, J=9.2, 7.0 Hz), 7.29-7.43 (m, 5H), 8.04 (dd, 1H, J=8.3, 2.2 Hz), 8.12 (d, 1H, J=7.9 Hz), 8.57 (d, 1H, J=8.3 Hz), 8.67 (s, 1H); ES-MS m/z 588 (M+1).

### EXAMPLE 28

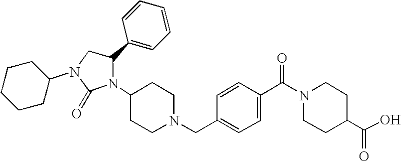

### COMPOUND 28: 1-{4-[4-((R)-3-Cyclohexyl-2-oxo-5-phenyl-imidazolidin-1-yl)-piperidin-1-ylmethyl]-benzoyl}-piperidine-4-carboxylic acid

Following General Procedure E: to a solution of 4-(hydroxymethyl)benzoic acid (310 mg, 2.04 mmol) and ethyl isonipecotate (312 mg, 1.98 mmol) in DMF (3.5 mL) was added DIPEA (0.4 mL, 2.30 mmol), HOBt (284 mg, 2.10 mmol) and EDC (441 mg, 2.30 mmol) and the reaction stirred overnight. Purification of the crude product gave the desired amide (0.43 g, 75%) as a pale brown oil. Following General Procedure G: to a solution of the alcohol from above (60 mg, 0.21 mmol) and DIPEA (0.2 mL, 1.15 mmol) in CH₂Cl₂ (5 mL) at −78° C. was added MsCl (0.05 mL, 0.65 mmol) and the reaction stirred at −78° C. for 15 min before warming to room temperature and stirring for an additional 15 min. The reaction was worked up as usual to afford 1-(4-methanesulfonyloxymethyl-benzoyl)-piperidine-4-carboxylic acid ethyl ester (108 mg) as a brown oil. ¹H NMR (CDCl₃) δ 1.23 (t, 3H, J=6 Hz), 1.55-2.08 (m, 4H), 2.50-2.57 (m, 1H), 2.97 (s, 3H), 3.02-3.09 (m, 2H), 3.62-3.67 (m, 2H), 4.14 (q, 2H, J=6 Hz), 5.24 (s, 2H), 7.38-7.46 (m, 4H).

Following General Procedure G: a suspension of (R)-1-cyclohexyl-4-phenyl-3-piperidin-4-yl-imidazolidin-2-one (68 mg, 0.21 mmol), K₂CO₃ (116 mg, 0.84 mmol) and the above mesylate (108 mg, 0.2 mmol) in CH₃CN (5 mL) was stirred at 60° C. overnight. Purification gave 1-{4-[4-((R)-3-cyclohexyl-2-oxo-5-phenyl-imidazolidin-1-yl)-piperidin-1-ylmethyl]-benzoyl}-piperidine-4-carboxylic acid ethyl ester (95 mg, 75%) as a white foam.

Following general procedure H, the above ester (133 mg, 0.22 mmol) in THF/H₂O (1:1, 4 mL) and LiOH—H₂O (115 mg, 2.74 mmol) afforded COMPOUND 28 (92 mg, 73%) as a white solid. ¹H NMR (CD₃OD) δ 1.05-1.09 (m, 1H), 1.34-1.95 (m, 17H), 2.30-2.36 (m, 1H), 2.57-2.67 (m, 1H), 2.97-3.31 (m, 4H), 3.35-3.47 (m, 2H), 3.62-3.80 (m, 4H), 4.26 (s, 2H), 4.37-4.48 (m, 1H), 4.73 (dd, 1H, J=9.3, 7.2 Hz), 7.33-7.40 (m, 5H), 7.47 (d, 2H, J=8.1 Hz), 7.57 (d, 2H, J=8.1 Hz); ¹³C NMR (CDCl₃) δ 26.89, 27.09, 27.18, 27.96, 28.65, 29.47, 30.22, 31.30, 31.64, 42.21, 43.11, 49.83, 51.24, 53.35, 53.43, 58.34, 60.78, 128.48, 128.98, 129.98, 130.61, 132.64, 133.32, 139.09, 143.73, 162.04, 171.83, 178.31; ES-MS m/z 573 (M+1). Anal. Calcd. for C₃₄H₄₄N₄O₄.13H₂O.2.4CH₂Cl₂: C, 43.10; H, 7.47; N, 5.52. Found: C, 43.17; H, 7.42; N, 5.80.

### EXAMPLE 29

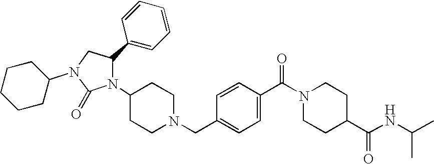

### COMPOUND 29: 1-{4-[4-((R)-3-Cyclohexyl-2-oxo-5-phenyl-imidazolidin-1-yl)-piperidin-1-ylmethyl]-benzoyl}-piperidine-4-carboxylic acid isopropylamide

1-(4-Hydroxymethyl-benzoyl)-piperidine-4-carboxylic acid ethyl ester (287 mg, 1.0 mmol) was dissolved in CH₂Cl₂ (10 mL) and cooled to 0° C. Triethylamine (0.16 mL, 1.4 mmol) and methanesulfonyl chloride (84 μL, 1.1 mmol) were added. The reaction was kept stirring at 0° C. for 40 minutes and then a saturated aqueous NaCl solution (10 mL) was added. Standard work-up afforded the residue, which was immediately dissolved in CH₃CN (8 mL) and treated with (R)-1-cyclohexyl-4-phenyl-3-piperidin-4-yl-imidazolidin-2-one (0.32 g, 1.0 mmol), as described in general procedure G. This gave, after chromatographic purification on silica gel (NH₃/Et₂O), 1-{4-[4-((R)-3-cyclohexyl-2-oxo-5-phenyl-imidazolidin-1-yl)-piperidin-1-ylmethyl]-benzoyl}-piperidine-4-carboxylic acid ethyl ester as a white solid (0.42 g, 71%).

Following general procedure H, the ester above (0.42 g, 0.70 mmol) afforded 1-{4-[4-((R)-3-cyclohexyl-2-oxo-5-phenyl-imidazolidin-1-yl)-piperidin-1-ylmethyl]-benzoyl}-piperidine-4-carboxylic acid as a white solid (0.39 g, 98%).

Following general procedure F, the above acid (40 mg, 70 μmol) was coupled with isopropylamine (16 μL, 0.18 mmol). After standard work-up and chromatographic purification on silica gel (NH₃/Et₂O), COMPOUND 29 was obtained as white solid (25 mg, 58%). ¹H NMR (CDCl₃) δ 1.00 (q, 1H, J=12.0 Hz), 1.14 (d, 6H, J=6.9 Hz), 1.20-1.40 (m, 7H), 1.63 (m, 2H), 1.73 (m, 6H), 1.75-2.04 (m, 4H), 2.28 (m, 1H), 2.67 (d, 1H, J=10.8 Hz), 2.83 (d, 1H, J=10.8 Hz), 2.90 (m, 2H), 3.04 (m, 1H), 3.41 (s, 2H), 3.63 (t, 2H, J=9.0 Hz), 3.76 (m, 1H), 4.09 (sept, 1H, J=7.5 Hz), 4.57 (m, 1H), 4.65 (s, 1H), 5.26 (m, 1H), 7.20-7.36 (m, 9H); ES-MS m/z 614 (M+H).

### EXAMPLE 30

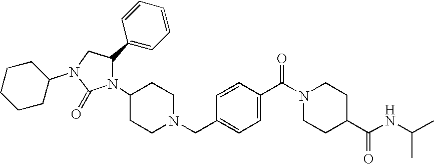

### COMPOUND 30: 4-{4-[4-((R)-3-Cyclohexyl-2-oxo-5-phenyl-imidazolidin-1-yl)-piperidin-1-ylmethyl]-benzoyl}-piperazine-1-carboxylic acid isopropylamide

Following general procedure E: a solution of 4-[4-((R)-3-cyclohexyl-2-oxo-5-phenyl-imidazolidin-1-yl)-piperidin-1-ylmethyl]-benzoic acid (183 mg, 0.40 mmol), 1-Boc-piperazine (94 mg, 0.50 mmol), EDCI (105 mg, 0.55 mmol), HOBT (87 mg, 0.64 mmol) and NMM (0.10 mL, 0.91 mmol) in DMF (2.5 mL) was stirred at room temperature for 17.5 hours to give 4-{4-[4-((R)-3-cyclohexyl-2-oxo-5-phenyl-imidazolidin-1-yl)-piperidin-1-ylmethyl]-benzoyl}-piperazine-1-carboxylic acid tert-butyl ester as a white foam (194 mg, 78%).

Following general procedure C, the tert-butyl carbamate (174 mg, 0.28 mmol) gave (R)-1-cyclohexyl-4-phenyl-3-{1-[4-(piperazine-1-carbonyl)-benzyl]-piperidin-4-yl}-imidazolidin-2-one as a white foam (111 mg, 76%).

A solution of the piperazine (35 mg, 0.066 mmol) and isopropyl isocyanate (8 mg, 0.092 mmol) in CH₂Cl₂ (0.50 mL) was stirred at 50° C. for 15.5 hours. The solvent was removed under reduced pressure and the residue was purified by flash column chromatography on silica (CH₂Cl₂/MeOH, 19:1), giving COMPOUND 30 as an off-white foam (23.8 mg, 59%). ¹H NMR (CDCl₃) δ 0.93-1.09 (m, 1H), 1.16 (d, 6H, J=6.2 Hz), 1.21-1.46 (m, 6H), 1.58-1.81 (m, 6H), 1.84-2.06 (m, 3H), 2.64-2.72 (m, 1H), 2.82-2.90 (m, 1H), 3.03 (dd, 1H, J=8.4, 7.2 Hz), 3.32-3.81 (m, 12H), 3.63 (t, 1H, J=9.0 Hz), 3.90-4.05 (m, 1H), 4.23 (d, 1H, J=6.9 Hz), 4.56 (dd, 1H, J=9.2, 6.9 Hz), 7.23-7.38 (m, 9H); ES-MS m/z 615 (M+H).

### EXAMPLE 31

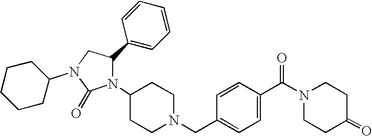

### COMPOUND 31: 1-{4-[4-((R)-3-Cyclohexyl-2-oxo-5-phenyl-imidazolidin-1-yl)-piperidin-1-ylmethyl]-benzoyl}-piperidin-4-one

A mixture of (R)-1-cyclohexyl-3-{1-[4-(4-hydroxy-piperidine-1-carbonyl)-benzyl]-piperidin-4-yl}-4-phenyl-imidazolidin-2-one (COMPOUND 21) (105 mg, 0.19 mmol), NMO (29 mg, 0.25 mmol) and TPAP (11 mg, 0.031 mmol) in CH₂Cl₂ (1.2 mL) was stirred at room temperature for 70 minutes. The reaction mixture was purified directly by flash column chromatography on silica (CH₂Cl₂/MeOH, 29:1), giving COMPOUND 31 as a white foam (83.7 mg, 80%). ¹H NMR (CDCl₃) δ 0.94-1.09 (m, 1H), 1.17-1.45 (m, 6H), 1.58-1.82 (m, 6H), 1.84-2.07 (m, 3H), 2.37-2.59 (m, 4H), 2.63-2.71 (m, 1H), 2.82-2.89 (m, 1H), 3.03 (dd, 1H, J=8.6, 7.1 Hz), 3.43 (s, 2H), 3.60-4.03 (m, 7H), 4.57 (dd, 1H, J=9.3, 6.5 Hz), 7.24-7.40 (m, 9H); ES-MS m/z 543 (M+H).

### EXAMPLE 32

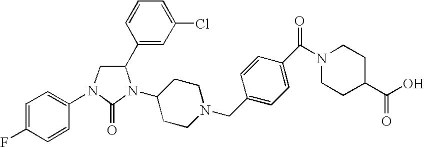

### COMPOUND 32: 1-(4-{4-[5-(3-Chloro-phenyl)-3-(4-fluoro-phenyl)-2-oxo-imidazolidin-1-yl]-piperidin-1-ylmethyl}-benzoyl)-piperidine-4-carboxylic acid

Following general procedure E, [1-(3-chloro-phenyl)-2-hydroxy-ethyl]-carbamic acid tert-butyl ester (see EXAMPLE 164) (1.00 g, 3.50 mmol) was coupled with 4-fluoro-phenylamine (0.400 g, 3.60 mmol) in the presence of EDCI (1.01 g, 5.25 mmol), HOBT (0.709 g, 5.25 mmol), and DIPEA (0.903 g, 7.00 mmol) in CH₂Cl₂ (50 mL) to give [(3-chloro-phenyl)-(4-fluoro-phenylcarbamoyl)-methyl]-carbamic acid tert-butyl ester (0.789 g, 59%).

Following general procedure C, the above product (0.789 g, 2.10 mmol), was treated with TFA (3 mL) in CH₂Cl₂ (10 mL) for 1 h to give 2-amino-2-(3-chloro-phenyl)-N-(4-fluoro-phenyl)-acetamide (0.425 g, 73%).

To a mixture of above product (0.425 g, 1.53 mmol) in dry THF (50 mL) was added BH₃-THF complex (1.0 M in THF, 4.6 mL, 4.6 mmol). The mixture was heated at reflux for 3 h. After cooling, methanol (10 mL) was added and the mixture was refluxed for another 15 min. Standard work-up gave 1-(3-chloro-phenyl)-N′2′-(4-fluoro-phenyl)-ethane-1,2-diamine (0.404 g, 100%).

Following general procedure A, a solution of above product (0.404 g, 1.53 mmol), 1-Boc-4-piperidone (0.304 g, 1.53 mmol), and NaBH(OAc)₃ (0.453 g, 2.14 mmol) in CH₂Cl₂ (5 mL) was stirred at room temperature for 17 h to give, after purifying by column chromatography, 4-[1-(3-chloro-phenyl)-2-(4-fluoro-phenylamino)-ethylamino]-piperidine-1-carboxylic acid tert-butyl ester (0.485 g, 71%).

Following general procedure K, to a solution of above product (0.485 g, 1.08 mmol) and pyridine (175 μL, 2.16 mmol) in dry dichloromethane (5 mL) at 0° C. was added triphosgene (128 mg, 0.432 mmol) in portions. After stirring at 0° C. for 30 min, the mixture was stirred at rt for another 2 h. The solvent was removed by evaporation and the residue was purified by column chromatography on silica gel (3:1, hexane/EtOAc) to give 4-[5-(3-chloro-phenyl)-3-(4-fluoro-phenyl)-2-oxo-imidazolidin-1-yl]-piperidine-1-carboxylic acid tert-butyl ester (0.303 g, 59%).

Following general procedure C, the above product (0.303 g, 0.640 mmol) was treated with TFA (1 mL) in CH₂Cl₂ (3 mL) for 1 h to give 4-(3-chloro-phenyl)-1-(4-fluoro-phenyl)-3-piperidin-4-yl-imidazolidin-2-one (0.239 g, 100%). ¹H NMR (CDCl₃) δ 1.11-1.25 (m, 1H), 1.53 (d, 1H, J=11.1 Hz), 1.71-1.87 (m, 2H), 2.00 (s, 1H), 2.47-2.65 (m, 2H), 2.93 (d, 1H, J=12.3 Hz), 3.09 (d, 1H, J=12.3 Hz), 3.79 (m, 1H), 4.11 (t, 1H, J=9.3 Hz), 4.72 (dd, 1H, J=9.6, 5.7 Hz), 6.99 (t, 2H, J=8.7 Hz), 7.23-7.30 (m, 3H), 7.36 (s, 1H), 7.45 (d, 2H, J=9.0, 4.8 Hz).

Following general procedure G: to a solution of 4-(3-chlorophenyl)-1-(4-fluoro-phenyl)-3-piperidin-4-yl-imidazolidin-2-one (80.2 mg, 0.215 mmol) in CH₃CN (3.5 mL) was added 1-(4-methanesulfonyloxymethyl-benzoyl)-piperidine-4-carboxylic acid ethyl ester (see EXAMPLE 28) (79.2 mg, 0.215 mmol) and K₂CO₃ (119 mg, 0.86 mmol). The mixture was stirred at 60° C. for 20 h. Standard work-up and purification afforded 1-(4-{4-[5-(3-chloro-phenyl)-3-(4-fluoro-phenyl)-2-oxo-imidazolidin-1-yl]-piperidin-1-ylmethyl}-benzoyl)-piperidine-4-carboxylic acid ethyl ester (73.5 mg, 53%).

Following general procedure H, the above ester (73.5 mg, 0.114 mmol) gave COMPOUND 32 as a yellow solid (70.5 mg, 100%). ¹H NMR (CD₃OD) δ 1.57-1.77 (m, 3H), 1.77-1.95 (m, 4H), 2.04 (m, 1H), 2.31 (m, 1H), 2.60-2.76 (m, 3H), 3.00-3.27 (m, 3H), 3.58-3.69 (m, 4H), 4.03 (s, 2H), 4.27(m, 1H), 4.45 (m, 1H), 4.92 (m, 1H), 7.04-7.11 (m, 2H), 7.41-7.55 (m, 9H), 7.90 (m, 1H); ¹³C NMR (CD₃OD) δ 28.61, 29.39, 29.60, 30.34, 42.66, 43.18, 53.32, 53.58, 53.67, 54.17, 57.01, 61.66, 79.89, 116.60, 116.90, 121.86, 121.96, 126.90, 128.74, 128.83, 130.30, 132.39, 132.67, 134.78, 136.40, 137.80, 138.57, 145.72, 159.07, 159.61, 172.03; ES-MS m/z 619 (M+H). Anal. Calcd. for C₃₄H₃₆ClN₄O₄F.1.6CH₂Cl₂.0.5H₂O: C, 55.96; H, 5.30; N, 7.33. Found: C, 55.78; H, 5.27; N, 7.42.

Examples 33 to 35 were prepared following the scheme illustrated below. RCHO is as defined in the table.

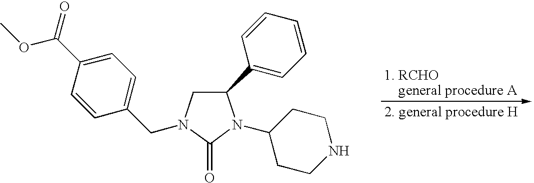

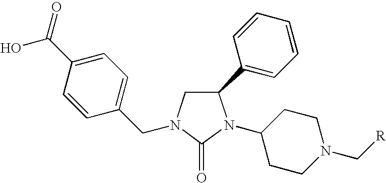

| Example | RCHO |
|---|---|
| 33 | 4-formyl-N-isopropyl-benzamide (see EXAMPLE 3) |
| 34 | 4-formyl-N-(tetrahydro-pyran-4-yl)-benzamide |
| 35 | quinoline-6-carbaldehyde |

### EXAMPLE 33

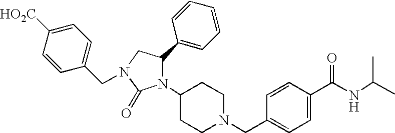

### COMPOUND 33: 4-{(R)-3-[1-(4-Isopropylcarbamoyl-benzyl)-piperidin-4-yl]-2-oxo-4-phenyl-imidazolidin-1-ylmethyl}-benzoic acid

COMPOUND 33 was isolated as a light yellow solid (49.0 mg, 72% over 2 steps). ¹H NMR (MeOH-d₄) δ 1.24 (d, 6H, J=6.6 Hz), 1.46-1.67 (m, 2H), 1.71-1.80 (m, 1H), 2.10-2.24 (m, 1H), 2.29-2.44 (m, 2H), 2.94-3.13 (m, 3H), 3.52-3.63 (m, 1H), 3.65 (t, 1H, J=9.4 Hz), 3.77 (s, 2H), 4.19 (septet, 1H, J=6.6 Hz), 4.45 (s, 2H), 4.74 (dd, 1H, J=9.1, 6.8 Hz), 7.25-7.37 (m, 7H), 7.41 (d, 2H, J=7.8 Hz), 7.78 (d, 2H, J=8.3 Hz), 7.96 (d, 2H, J=7.8 Hz); ES-MS m/z 555 (M+H).

### EXAMPLE 34

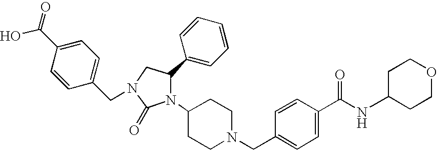

### COMPOUND 34: 4-((R)-2-Oxo-4-phenyl-3-{1-[4-(tetrahydro-pyran-4-ylcarbamoyl)-benzyl]-piperidin-4-yl}-imidazolidin-1-ylmethyl)-benzoic acid

Following general procedure E: a solution of tetrahydro-pyran-4-ylamine (prepared according to literature procedure: Renhowe, Paul A, patent, US2002/137939 A1) (100 mg, 0.99 mmol), 4-formylbenzoic acid (182 mg, 1.21 mmol), EDCI (249 mg, 1.30 mmol), HOBT (202 mg, 1.49 mmol), NMM (0.25 mL, 2.3 mmol) and DMF (1.0 mL) in CH₂Cl₂ (5.0 mL) was stirred at room temperature for 17 hours giving 4-formyl-N-(tetrahydro-pyran-4-yl)-benzamide as a yellow solid (151 mg, 65%). ¹H NMR (CDCl₃) δ 1.59 (qd, 2H, J=11.9, 4.0 Hz), 1.91-2.01 (m, 2H), 3.49 (t, 2H, J=11.9 Hz), 3.92-4.01 (m, 2H), 4.12-4.25 (m, 1H), 6.69 (br s, 1H), 7.89 (d, 2H, J=8.14 Hz), 7.93 (d, 2H, J=8.1 Hz), 10.03 (s, 1H).

COMPOUND 34 was isolated as an off-white solid (23.0 mg, 43% over 2 steps). ¹H NMR (MeOH-d₄) δ 1.42-1.78 (m, 6H), 1.83-1.93 (m, 2H), 2.08-2.38 (m, 3H), 2.90-3.10 (m, 3H), 3.46-3.60 (m, 2H), 3.65 (t, 1H, J=9.2 Hz), 3.73 (s, 2H), 3.93-4.02 (m, 2H), 4.04-4.15 (m, 1H), 4.45 (s, 2H), 4.75 (dd, 1H, J=9.4, 6.8 Hz), 7.25-7.37 (m, 7H), 7.41 (d, 2H, J=8.0 Hz), 7.79 (d, 2H, J=8.2 Hz), 7.96 (d, 2H, J=8.2 Hz); ES-MS m/z 597 (M+H).

### EXAMPLE 35

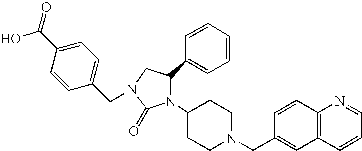

### COMPOUND 35: 4-[(R)-2-Oxo-4-phenyl-3-(1-quinolin-6-ylmethyl-piperidin-4-yl)-imidazolidin-1-ylmethyl]-benzoic acid

To a solution of 6-quinoline carboxylic acid (200 mg, 1.15 mmol) in THF at room temperature was added LAH (131 mg, 3.46 mmol). The reaction was stirred at room temperature for 2 h and then quenched with water (130 μL), NaOH (15%, aq) (130 μL) and water (275 μL). The resulting suspension was stirred at room temperature for 30 min and the white solid was filtered off. The solution was dried over Na₂SO₄, filtered and concentrated to afford a mixture of alcohol and over reduction (aromatic ring) products. The crude material was used as is for the following step.

To a solution of the above alcohol (1.15 mmol) in dichloromethane (3 mL) was added 4-methylmorpholine-N-oxide (337 mg, 2.87 mmol) and TPAP (40 mg, 0.12 mmol). The reaction was stirred at room temperature for 2 h and the solvent was removed. The residue was purified by flash chromatography on silica gel column (10% ether in CH₂Cl₂) to afford quinoline-6-carbaldehyde (97 mg, 54% over 2 steps).

COMPOUND 35 was isolated as a white solid (29 mg, 49% over 2 steps). ¹H NMR (CD₃OD) δ 0.80-2.40 (m, 5H), 2.51-2.74 (m, 2H), 2.95-3.28 (m, 3H), 3.67 (t, 1H, J=9.2 Hz), 4.08 (s, 2H), 4.45 (s, 2H), 4.73-4.85 (m, 1H), 7.22-7.43 (m, 5H), 7.57 (dd, 1H, J=8.3, 4.3 Hz), 7.81 (d, 1H, J=8.7 Hz), 7.91-8.10 (m, 5H), 8.38 (d, 1H, J=8.3 Hz), 8.46 (s, 1H), 8.87 (d, 1H, J=4.5 Hz); ES-MS m/z 521 (M+1).

### EXAMPLE 36

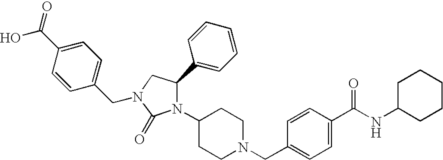

### COMPOUND 36: 4-{(R)-3-[1-(4-cyclohexylcarbamoyl-benzyl)-piperidin-4-yl]-2-oxo-4-phenyl-imidazolidin-1-ylmethyl}-benzoic acid

To a solution of 4-(bromomethyl)benzoic acid (233 mg, 1.08 mmol) and NMM (0.16 mL, 1.5 mmol) in THF (5 mL) was added isobutyl chloroformate (0.15 mL, 1.2 mmol) and the resulting suspension was stirred at room temperature for 5 minutes. A solution of cyclohexylamine (0.15 mL, 1.3 mmol) in THF (2 mL) was added and the reaction was stirred for an additional 17 hours. Standard work-up and purification by flash column chromatography on silica (CH₂Cl₂/Et₂O, 19:1) gave 4-bromomethyl-N-cyclohexyl-benzamide as a light yellow solid (164 mg, 51%). ¹H NMR (CDCl₃) δ 1.05-2.07 (m, 10H), 3.89-4.02 (m, 1H), 4.49 (s, 2H), 5.93-6.05 (m, 1H), 7.42 (d, 2H, J=7.8 Hz), 7.72 (d, 2H, J=7.8 Hz).

Following general procedure G: a solution of the bromide (0.12 mmol), 4-((R)-2-oxo-4-phenyl-3-piperidin-4-yl-imidazolidin-1-ylmethyl)-benzoic acid methyl ester (53 mg, 0.14 mmol) and DIPEA (35 μL, 0.20 mmol) in CH₃CN (1.0 mL) was stirred at 60° C. for 16.5 hours to give 4-{(R)-3-[1-(4-cyclohexylcarbamoyl-benzyl)-piperidin-4-yl]-2-oxo-4-phenyl-imidazolidin-1-ylmethyl}-benzoic acid methyl ester as an off-white foam (63.1 mg, 86%).

Following general procedure H: a solution of the methyl ester (47 mg, 0.077 mmol) and 10M NaOH (0.15 mL, 1.5 mmol) in MeOH (1.5 mL) was stirred at 60° C. for 2.5 hours giving COMPOUND 36 as an off-white solid (43.7 mg, 95%). ¹H NMR (MeOH-d₄) δ 1.15-1.86 (m, 11H), 1.89-1.97 (m, 2H), 2.09-2.24 (m, 1H), 2.27-2.43 (m, 2H), 2.92-3.14 (m, 3H), 3.51-3.63 (m, 1H), 3.65 (t, 1H, J=9.4 Hz), 3.77 (s, 2H), 3.78-3.89 (m, 1H), 4.45 (s, 2H), 4.75 (dd, 1H, J=9.0, 7.2 Hz), 7.25-7.37 (m, 7H), 7.41 (d, 2H, J=8.3 Hz), 7.77 (d, 2H, J=8.5 Hz), 7.96 (d, 2H, J=8.3 Hz); ES-MS m/z 595 (M+H).

### EXAMPLE 37

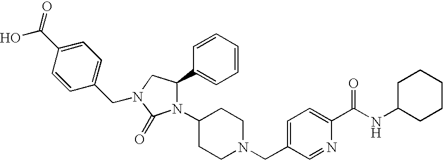

### COMPOUND 37: 4-{(R)-3-[1-(6-Cyclohexylcarbamoyl-pyridin-3-ylmethyl)-piperidin-4-yl]-2-oxo-4-phenyl-imidazolidin-1-ylmethyl}-benzoic acid

To a solution of 2-bromo-5-methylpyridine (500 mg, 2.9 mmol) in CCl₄ (15 mL) was added NBS (569 mg, 3.19 mmol) and VAZO™ (71 mg, 0.29 mmol). The reaction was stirred at reflux for 2.5 h then cooled down to room temperature and washed with saturated aqueous NaHCO₃. The organic material was extracted with CH₂Cl₂, dried over Na₂SO₄, filtered and concentrated under reduced pressure. The crude 2-bromo-5-bromomethylpyridine was used as is for the following step.

A solution of 1,4-dioxa-8-aza-spiro[4.5]decane (372 μL, 2.9 mmol) and the above bromide (2.9 mmol) in acetonitrile (15 mL) was refluxed for 30 min. The reaction was quenched with saturated aqueous NaHCO₃. The mixture was extracted with CH₂Cl₂, dried over Na₂SO₄, filtered and concentrated under reduced pressure. The crude material was purified by flash chromatography on silica gel (5% MeOH in CH₂Cl₂) to give 8-(6-bromopyridin-3-ylmethyl)-1,4-dioxa-8-azaspiro[4.5]decane (503 mg, 55% over 2 steps).

To a solution of the above bromide (257 mg, 0.82 mmol) in ether (5 mL) at −78° C. was added drop wise n-BuLi/Hexane [2.5M] (393 μL, 0.98 mmol). The reaction was stirred at −78° C. for 30 min and then gaseous CO₂ was bubbled in the solution for a 30 min period. The reaction mixture was warmed to room temperature and stirred for an additional hour. The solution was treated with HCl (1M, aq) and the aqueous layer was washed with ether. The aqueous layer was concentrated under reduced pressure and the crude 5-(1,4-dioxa-8-azaspiro[4.5]dec-8-ylmethyl)-pyridine-2-carboxylic acid hydrochloride (287 mg) was used as is for the following step.

Using general procedure E, the above acid (145 mg, 0.52 mmol) and cyclohexylamine (60 μL, 0.52 mmol) in DMF (3 mL) afforded 5-(1,4-dioxa-8-aza-spiro[4.5]dec-8-ylmethyl)-pyridine-2-carboxylic acid cyclohexylamide (85 mg, 29% over 2 steps).

A solution of the above amide (85 mg, 0.24 mmol) in a mixture of HCl (6N, aq)/acetone (1:1, 5 mL) was stirred at reflux for 18 h. The volatiles were removed under reduced pressure and the resulting aqueous solution was basified to pH 8 with NaOH (3N, aq). The solution was extracted with CH₂Cl₂, dried over Na₂SO₄, filtered and concentrated. The crude material was purified by flash chromatography on silica gel (2% MeOH in CH₂Cl₂) to afford 5-(4-oxopiperidin-1-ylmethyl)-pyridine-2-carboxylic acid cyclohexylamide (32 mg, 43%).

Using general procedure A, 4-[((R)-2-amino-2-phenyl-ethylamino)-methyl]-benzoic acid methyl ester (32 mg, 0.11 mmol) and 5-(4-oxopiperidin-1-ylmethyl)-pyridine-2-carboxylic acid cyclohexylamide (32 mg, 0.10 mmol) afforded 4-({(R)-2-[1-(6-cyclohexylcarbamoylpyridin-3-ylmethyl)-piperidin-4-ylamino]-2-phenyl-ethylamino}-methyl)-benzoic acid methyl ester (64 mg). The crude material was used as is for the following step.

Following general procedure K: to a cooled (0° C.) solution of the above diamine (64 mg, 0.11 mmol) and pyridine (18 μL, 0.22 mmol) in dry dichloromethane (3 mL) was slowly added triphosgene (16 mg, 0.05 mmol). The ice bath was removed and the mixture was stirred at ambient temperature for 2 h. Standard work-up and purification afforded 4-{(R)-3-[1-(6-cyclohexylcarbamoylpyridin-3-ylmethyl)-piperidin-4-yl]-2-oxo-4-phenylimidazolidin-1-ylmethyl}-benzoic acid methyl ester (36 mg, 54%).

Using general procedure H, the above methyl ester (30 mg, 0.05 mmol) gave COMPOUND 37 as a white solid (29 mg, 100%). ¹H NMR (CD₃OD) δ 1.19-2.06 (m, 12H), 2.22-2.44 (m, 1H), 2.64-2.84 (m, 2H), 3.04 (t, 1H, J=6.9 Hz), 3.15-3.37 (m, 2H), 3.56-3.72 (m, 1H), 3.68 (t, 1H, J=9.2 Hz), 3.80-3.95 (m, 1H), 4.11 (s, 2H), 4.46 (s, 2H), 4.73-4.96 (m, 2H), 7.26-7.47 (m, 6H), 7.95-8.14 (m, 4H), 8.56 (d, 1H, J=8.4 Hz), 8.66 (s, 1H); ES-MS m/z 596 (M+1).

Examples 38 to 43 were prepared following the scheme illustrated below. RCHO is as defined in the table.

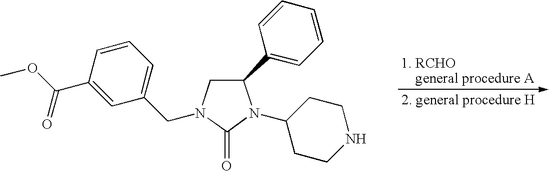

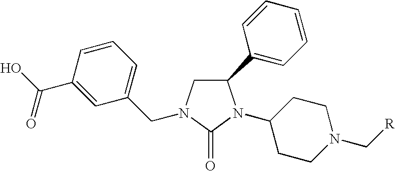

| Example | RCHO |
|---|---|
| 38 | 5,6,7,8-tetrahydroquinoline-6-carboxaldehyde |
| 39 | 6-(tert-butyl)-2-methylpyridine-3-carboxaldehyde |
| 40 | 5-methyl-imidazo[1,2-a]pyridine-6-carbaldehyde |
| 41 | 6-cyclohexyl-2-methyl-pyridine-3-carbaldehyde |
| 42 | 2-methyl-6-(tetrahydro-pyran-4-yl)-pyridine-3-carbaldehyde |
| 43 | 4-ethyl-2-isopropylthiazole-5-carbaldehyde |

### EXAMPLE 38

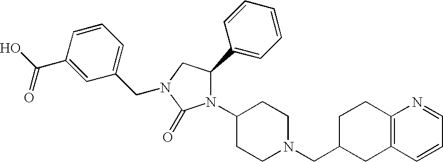

### COMPOUND 38: 3-{(R)-2-Oxo-4-phenyl-3-[1-(5,6,7,8-tetrahydro-quinolin-6-ylmethyl)-piperidin-4-yl]-imidazolidin-1-ylmethyl}-benzoic acid

A suspension of 6-quinolinecarboxylic acid (855 mg, 4.94 mmol) and SOCl₂ (1.0 mL, 13.7 mmol) in CH₂Cl₂ (20 mL) was stirred at room temperature for 45 minutes. The solvent was removed under reduced pressure, the residue was dissolved in MeOH (20 mL) and this solution was stirred at reflux under nitrogen for 75 minutes. Standard work-up gave crude methyl quinoline-6-carboxylate as a beige powder (796 mg, 86%).

To a mixture of the quinoline (394 mg, 2.10 mmol) and PtO₂.H₂O (31 mg, 0.14 mmol) under nitrogen was added TFA (6.5 mL) (McEachern, E. J.; Bridger, G. J.; Skupinska, K. A.; Skerlj, R. T. U.S. Pat. Appl. 20030114679). The flask was flushed with hydrogen and the reaction was stirred at 60° C. under hydrogen (balloon) for 5 hours. Once cooled, the mixture was carefully neutralized with 10M NaOH (8 mL), diluted with saturated aqueous NaHCO₃ (25 mL) and was extracted with CH₂Cl₂ (25 mL×3). The organic solution was dried (Na₂SO₄), filtered and concentrated under reduced pressure. Purification by flash column chromatography on silica (hexane/EtOAc, 4:1, increased to 1:1) gave methyl 5,6,7,8-tetrahydroquinoline-6-carboxylate as a yellow oil (52.3 mg, 13%) along with methyl 1,2,3,4-tetrahydroquinoline-6-carboxylate (149 mg, 37%) and recovered starting material (129 mg, 33%).

Data for methyl 5,6,7,8-tetrahydroquinoline-6-carboxylate: ¹H NMR (CDCl₃) δ 1.91-2.06 (m, 1H), 2.25-2.34 (m, 1H), 2.74-2.84 (m, 1H), 2.90-3.10 (m, 4H), 3.73 (s, 3H), 7.05 (dd, 1H, J=7.4, 4.8 Hz), 7.39 (d, 1H, J=7.5 Hz), 8.37 (d, 1H, J=4.8 Hz).

Data for methyl 1,2,3,4-tetrahydroquinoline-6-carboxylate: ¹H NMR (CDCl₃) δ 1.88-1.96 (m, 2H), 2.76 (t, 2H, J=6.2 Hz), 3.35 (t, 2H, J=5.4 Hz), 3.83 (s, 3H), 4.33 (br. s, 1H), 6.38 (d, 1H, J=9.1 Hz), 7.62-7.66 (m, 2H).

A mixture of the 5,6,7,8-tetrahydroquinoline (52.3 mg, 0.27 mmol) and LiAlH₄ (13 mg, 0.34 mmol) in THF (1.0 mL) was stirred at room temperature for 30 minutes. The reaction was quenched with EtOAc (2 mL), was diluted with saturated aqueous NaHCO₃ (25 mL) and was extracted with CH₂Cl₂ (20 mL×3). The combined organic solution was dried (Na₂SO₄), filtered and concentrated under reduced pressure, giving 6-hydroxymethyl-5,6,7,8-tetrahydroquinoline as a cloudy oil (41.9 mg, 94%).

A mixture of the alcohol (41.9 mg, 0.26 mmol), TPAP (9.0 mg, 0.026 mmol) and NMO (34 mg, 0.29 mmol) in CH₂Cl₂ (1.7 mL) was stirred at room temperature for 1.5 hours. The reaction was purified directly by flash column chromatography on silica (CH₂Cl₂/MeOH, 29:1), giving 5,6,7,8-tetrahydroquinoline-6-carboxaldehyde as a cloudy oil (20.3 mg, 49%). ¹H NMR (CDCl₃) δ 1.87-1.98 (m, 1H), 2.29-2.39 (m, 1H), 2.70-2.80 (m, 1H), 2.92-3.12 (m, 4H), 7.07 (dd, 1H, J=7.9, 4.8 Hz), 7.43 (d, 1H, J=7.9 Hz), 8.38 (d, 1H, J=4.8 Hz), 9.81 (s, 1H).

COMPOUND 38 was isolated as an off-white powder (16.4 mg, 31% over 2 steps). ¹H NMR (MeOH-d₄) δ 1.15-1.40 (m, 2H), 1.48-1.62 (m, 1H), 1.67-1.90 (m, 3H), 2.03-2.21 (m, 2H), 2.27-2.40 (m, 1H), 2.44-2.61 (m, 2H), 2.68-2.79 (m, 2H), 2.86-3.09 (m, 3H), 3.17-3.29 (m, 1H), 3.44-3.74 (m, 3H), 4.42 (d, 1H, J=15.3 Hz), 4.49 (d, 1H, J=15.3 Hz), 4.74-4.82 (m, 1H), 7.13-7.21 (m, 1H), 7.27-7.48 (m, 7H), 7.49-7.56 (m, 1H), 7.86-7.98 (m, 2H), 8.22-8.30 (m, 1H); ES-MS m/z 525 (M+H).

### EXAMPLE 39

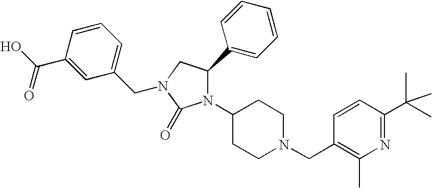

### COMPOUND 39: 3-{(R)-3-[1-(6-tert-Butyl-2-methyl-pyridin-3-ylmethyl)-piperidin-4-yl]-2-oxo-4-phenyl-imidazolidin-1-ylmethyl}-benzoic acid

To a stirred solution of ethyl 2-methylnicotinate (2.76 g, 16.71 mmol), trimethylacetic acid (8.53 g, 83.5 mmol) and silver nitrate (584 mg, 3.44 mmol) in 10% aqueous H₂SO₄ (17 mL) was added a solution of ammonium persulfate (7.72 g, 33.8 mmol) in H₂O (35 mL) (Bo, Y. Y.; Chakrabarti, P. P.; Chen, N.; Doherty, E. M.; Fotsch, C. H.; Han, N.; Kelly, M. G.; Liu, Q.; Norman, M. H.; Ognyanov, V. I.; Wang, X.; Zhu, J. U.S. Pat. Appl. 20030195201). The resulting mixture was stirred at room temperature for 2 hours. The reaction was neutralized to pH 10 with aqueous NH₄OH and was extracted with EtOAc (30 mL×3). The combined organic solution was washed with H₂O (50 mL), was dried (Na₂SO₄), filtered and concentrated under reduced pressure. Purification by flash column chromatography on silica (hexane/EtOAc, 4:1) gave ethyl 6-(tert-butyl)-2-methylpyridine-3-carboxylate as a pale yellow liquid (2.77 g, 75%).

A mixture of the ethyl ester (505 mg, 2.28 mmol) and LiAlH₄ (116 mg, 3.06 mmol) in THF (9.0 mL) was stirred at room temperature for 50 minutes. The reaction was quenched with H₂O (0.12 mL), 15% NaOH (0.12 mL) and H₂O (0.35 mL) and the resulting suspension was filtered through Celite®, washing the residue with EtOAc. The filtrate was concentrated under reduced pressure, giving crude 6-(tert-butyl)-2-methylpyridine-3-methanol as a pale yellow oil (459 mg, quantitative).

A mixture of the crude alcohol (1.14 mmol), TPAP (22 mg, 0.063 mmol) and NMO (263 mg, 2.24 mmol) in CH₂Cl₂ (8 mL) was stirred at room temperature for 30 minutes. The solvent was removed under reduced pressure and the residue was purified directly by flash column chromatography on silica (hexane/EtOAc, 4:1), giving 6-(tert-butyl)-2-methylpyridine-3-carboxaldehyde as a colourless liquid (136 mg, 67%). ¹H NMR (CDCl₃) δ 1.37 (s, 9H), 2.85 (s, 3H), 7.33 (d, 1H, J=8.2 Hz), 8.00 (d, 1H, J=8.2 Hz), 10.29 (s, 1H).

COMPOUND 39 was isolated as a white powder (26.5 mg, 65% over 2 steps). ¹H NMR (MeOH-d₄) δ 1.31 (s, 9H), 1.43-1.63 (m, 2H), 1.71-1.80 (m, 1H), 2.10-2.24 (m, 1H), 2.34-2.48 (m, 2H), 2.51 (s, 3H), 2.94-3.03 (m, 1H), 2.99 (dd, 1H, J=8.9, 7.1 Hz), 3.07-3.15 (m, 1H), 3.56-3.68 (m, 1H), 3.64 (t, 1H, J=9.2 Hz), 3.73 (s, 2H), 4.45 (s, 2H), 4.73 (dd, 1H, J=9.4, 6.9 Hz), 7.20 (d, 1H, J=8.0 Hz), 7.23-7.34 (m, 5H), 7.37-7.46 (m, 2H), 7.56 (d, 1H, J=8.2 Hz), 7.86-7.94 (m, 2H); ES-MS m/z 541 (M+H).

### EXAMPLE 40

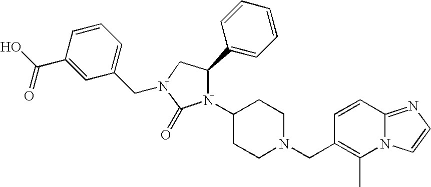

### COMPOUND 40: 3-{(R)-3-[1-(5-Methyl-imidazo[1,2-a]pyridin-6-ylmethyl)-piperidin-4-yl]-2-oxo-4-phenyl-imidazolidin-1-ylmethyl}-benzoic acid

To a solution of 2-amino-6-picoline (3.03 g, 28.0 mmol) in MeOH (85 mL) was slowly added NBS (5.07 g, 28.5 mmol) portion-wise as a solid. The resulting solution was stirred at room temperature for 30 minutes and then was concentrated under reduced pressure. Basic work-up and purification gave 6-amino-3-bromo-2-picoline as a white solid (4.49 g, 86%).

A mixture of bromoacetaldehyde diethyl acetal (2.3 mL, 15.3 mmol) in 1M HCl (10 mL) was stirred at 90° C. for 2 hours. The mixture was cooled to room temperature and NaHCO₃ (1.27 g, 15.1 mmol) was carefully added portion-wise as a solid, followed by 6-amino-3-bromo-2-picoline (935 mg, 5.00 mmol). The resulting mixture was stirred at 60° C. for another 40 minutes. Standard work-up and purification gave 6-bromo-5-methyl-imidazo[1,2-a]pyridine as a white solid (826 mg, 78%).

To a −78° C. mixture of the bromide (264 mg, 1.25 mmol) in THF (7.0 mL) under nitrogen was added dropwise n-BuLi (2.5M in hexanes, 0.50 mL, 1.3 mmol). The resulting solution was stirred at −78° C. for 15 minutes and then anhydrous DMF (0.15 mL, 1.9 mmol) was added. The reaction was stirred for an additional 1.5 hours, while slowly warming to room temperature. The solution was quenched with saturated aqueous NH₄Cl (25 mL) and the mixture was extracted with EtOAc (20 mL×3). The combined organic solution was washed with brine (30 mL), was dried (Na₂SO₄), filtered and concentrated under reduced pressure. Purification by flash column chromatography on silica (CH₂Cl₂/MeOH, 49:1) gave impure material which, following trituration with Et₂O (10 mL×3), supplied 5-methyl-imidazo[1,2-a]pyridine-6-carbaldehyde as an off-white solid (19.3 mg, 10%). ¹H NMR (CDCl₃) δ 2.97 (s, 3H), 6.89 (d, 1H, J=6.9 Hz), 7.46 (dd, 1H, J=8.6, 7.4 Hz), 7.64 (d, 1H, J=9.3 Hz), 8.40 (s, 1H), 9.90 (s, 1H).

COMPOUND 40 was isolated as an off-white powder (22.1 mg, 35% over 2 steps). ¹H NMR (MeOH-d₄) δ 1.27-1.42 (m, 1H), 1.49-1.58 (m, 1H), 1.65-1.73 (m, 1H), 1.94-2.09 (m, 3H), 2.75-2.84 (m, 1H), 2.89-2.97 (m, 1H), 3.00 (s, 3H), 3.04 (dd, 1H, J=8.6, 6.5 Hz), 3.47-3.60 (m, 1H), 3.67 (t, 1H, J=9.1 Hz), 3.77 (d, 1H, J=13.9 Hz), 3.84 (d, 1H, J=13.9 Hz), 4.45 (d, 1H, J=15.6 Hz), 4.51 (d, 1H, J=15.6 Hz), 4.77 (dd, 1H, J=9.0, 6.4 Hz), 6.73 (d, 1H, J=6.6 Hz), 7.23-7.52 (m, 10H), 7.91-7.98 (m, 2H); ES-MS m/z 524 (M+H).

### EXAMPLE 41

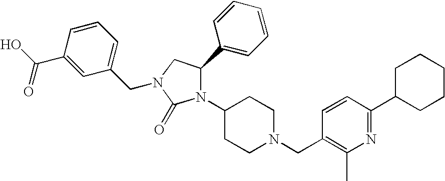

### COMPOUND 41: 3-{(R)-3-[1-(6-Cyclohexyl-2-methyl-pyridin-3-ylmethyl)-piperidin-4-yl]-2-oxo-4-phenyl-imidazolidin-1-ylmethyl}-benzoic acid

To a stirred solution of ethyl 2-methylnicotinate (425 mg, 2.57 mmol), cyclohexanecarboxylic acid (849 mg, 6.62 mmol) and silver nitrate (110 mg, 0.65 mmol) in 10% aqueous H₂SO₄ (3 mL) was added a solution of ammonium persulfate (1.21 g, 5.30 mmol) in H₂O (6 mL). The resulting mixture was stirred at room temperature for 1.5 hours. The reaction was made basic (pH 11) with aqueous NH₄OH and was extracted with EtOAc (25 mL×3). The combined organic solution was washed with brine (50 mL), was dried (Na₂SO₄), filtered and concentrated under reduced pressure. Purification by flash column chromatography on silica (hexane/EtOAc, 14:1) gave 6-cyclohexyl-2-methyl-nicotinic acid ethyl ester as colourless liquid (211 mg, 33%).

A mixture of the ethyl ester (199 mg, 0.80 mmol) and LiAlH₄ (35 mg, 0.92 mmol) in THF (4 mL) was stirred at room temperature for 60 minutes. The reaction was quenched by the addition of H₂O (0.05 mL), 15% NaOH (0.05 mL) and H₂O (0.15 mL). The resulting suspension was filtered through Celite®, washing with EtOAc and the filtrate was concentrated under reduced pressure, giving crude (6-cyclohexyl-2-methyl-pyridin-3-yl)-methanol as a white solid (168 mg, quantitative).

A mixture of the crude alcohol (0.80 mmol), NMO (155 mg, 1.32 mmol) and TPAP (25 mg, 0.071 mmol) in CH₂Cl₂ (4 mL) was stirred at room temperature for 60 minutes. The reaction mixture was purified directly by flash column chromatography on silica (CH₂Cl₂/Et₂O, 19:1), giving 6-cyclohexyl-2-methyl-pyridine-3-carbaldehyde as a colourless oil (126 mg, 77%). ¹H NMR (CDCl₃) δ 1.21-1.57 (m, 5H), 1.72-1.80 (m, 1H), 1.82-1.90 (m, 2H), 1.91-1.99 (m, 2H), 2.74 (tt, 1H, J=11.4, 3.3 Hz), 2.85 (s, 3H), 7.16 (d, 1H, J=8.2 Hz), 8.01 (d, 1H, J=8.2 Hz), 10.28 (s, 1H).

COMPOUND 41 was isolated as a white solid (43.0 mg, 69% over 2 steps). ¹H NMR (MeOH-d₄) δ 1.29-1.66 (m, 7H), 1.73-1.84 (m, 2H), 1.86-1.97 (m, 4H), 2.15 (qd, 1H, J=12.4, 3.8 Hz), 2.24-2.39 (m, 2H), 2.54 (s, 3H), 2.65-2.75 (m, 1H), 2.88-2.96 (m, 1H), 3.01-3.09 (m, 1H), 3.05 (dd, 1H, J=9.0, 6.7 Hz), 3.62 (tt, 1H, J=11.6, 3.7 Hz), 3.65 (s, 2H), 3.70 (t, 1H, J=9.4 Hz), 4.49 (s, 2H), 4.79 (dd, 1H, J=9.4, 6.8 Hz), 7.15 (d, 1H, J=7.9 Hz), 7.30-7.40 (m, 5H), 7.43-7.52 (m, 2H), 7.65 (d, 1H, J=7.9 Hz), 7.94 (d, 1H, J=7.5 Hz), 7.97 (d, 1H, J=1.8 Hz); ES-MS m/z 567 (M+H).

### EXAMPLE 42

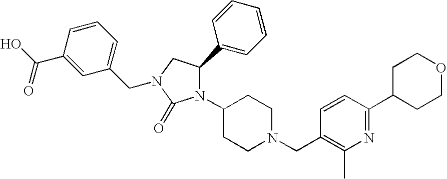

### COMPOUND 42: 3-((R)-3-{1-[2-Methyl-6-(tetrahydro-pyran-4-yl)-pyridin-3-ylmethyl]-piperidin-4-yl}-2-oxo-4-phenyl-imidazolidin-1-ylmethyl)-benzoic acid

A solution of methyl tetrahydro-2H-pyran-4-carboxylate (3.06 g, 21.2 mmol) and 4M NaOH (20 mL, 80 mmol) in MeOH (40 mL) was stirred at 60° C. for 2 hours. The organic solvent was removed under reduced pressure and the residual aqueous solution was acidified with concentrated HCl and extracted with CHCl₃ (30 mL×4). The combined organic solution was dried (Na₂SO₄), filtered and concentrated under reduced pressure, giving crude tetrahydro-2H-pyran-4-carboxylic acid as a white solid (2.55 g, 92%).

To a stirred solution of the crude carboxylic acid (2.55 g, 19.6 mmol), ethyl 2-methylnicotinate (835 mg, 5.05 mmol) and silver nitrate (262 mg, 1.54 mmol) in 10% aqueous H₂SO₄ (5 mL) was added a solution of ammonium persulfate (2.45 g, 10.7 mmol) in H₂O (10 mL). The resulting mixture was stirred at room temperature for 3 hours. The reaction was made basic (pH 11) with aqueous NH₄OH and was extracted with EtOAc (25 mL×2). The combined organic solution was washed with brine (30 mL), was dried (Na₂SO₄), filtered and concentrated under reduced pressure. Purification by flash column chromatography on silica (CH₂Cl₂/Et₂O, 4:1) gave 2-methyl-6-(tetrahydro-pyran-4-yl)-nicotinic acid ethyl ester as a colourless liquid (266 mg, 21%).

A mixture of the ethyl ester (266 mg, 1.07 mmol) and LiAlH₄ (45 mg, 1.19 mmol) in THF (5 mL) was stirred at room temperature for 60 minutes. The reaction was quenched by the addition of H₂O (0.05 mL), 15% NaOH (0.05 mL) and H₂O (0.15 mL). The resulting suspension was filtered through Celite®, washing with EtOAc and the filtrate was concentrated under reduced pressure. Purification by flash column chromatography on silica (CH₂Cl₂/MeOH, 29:1) gave [2-methyl-6-(tetrahydro-pyran-4-yl)-pyridin-3-yl]-methanol as a white solid (135 mg, 61%).

A mixture of the alcohol (135 mg, 0.65 mmol), NMO (127 mg, 1.08 mmol) and TPAP (16 mg, 0.045 mmol) in CH₂Cl₂ (3.5 mL) was stirred at room temperature for 60 minutes. The reaction mixture was purified directly by flash column chromatography on silica (CH₂Cl₂/Et₂O, 3:1), giving 2-methyl-6-(tetrahydro-pyran-4-yl)-pyridine-3-carbaldehyde as a beige solid (99.6 mg, 74%). ¹H NMR (CDCl₃) δ 1.83-1.98 (m, 4H), 2.86 (s, 3H), 2.95-3.07 (m, 1H), 3.48-3.62 (m, 2H), 4.07-4.13 (m, 2H), 7.18 (d, 1H, J=7.8 Hz), 8.05 (d, 1H, J=7.8 Hz), 10.29 (s, 1H).

COMPOUND 42 was isolated as a white solid (45.9 mg, 76% over 2 steps). ¹H NMR (MeOH-d₄) δ 1.41-1.55 (m, 1H), 1.56-1.64 (m, 1H), 1.71-1.94 (m, 5H), 2.07-2.37 (m, 3H), 2.54 (s, 3H), 2.86-3.09 (m, 3H), 3.05 (dd, 1H, J=8.9, 6.5 Hz), 3.54-3.65 (m, 3H), 3.64 (s, 2H), 3.70 (t, 1H, J=9.4 Hz), 4.04-4.12 (m, 2H), 4.47 (d, 1H, J=15.6 Hz), 4.52 (d, 1H, J=15.6 Hz), 4.79 (dd, 1H, J=9.4, 6.8 Hz), 7.15 (d, 1H, J=7.9 Hz), 7.29-7.40 (m, 5H), 7.43-7.53 (m, 2H), 7.64 (d, 1H, J=7.9 Hz), 7.92-7.98 (m, 2H); ES-MS m/z 569 (M+H).

### EXAMPLE 43

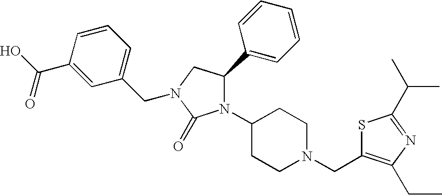

### COMPOUND 43: 3-{(R)-3-[1-(4-Ethyl-2-isopropyl-thiazol-5-ylmethyl)-piperidin-4-yl]-2-oxo-4-phenyl-imidazolidin-1-ylmethyl}-benzoic acid

A solution of isobutyramide (723 mg, 8.3 mmol) and Lawesson reagent (1.67 g, 4.2 mmol) in THF (8 mL) was stirred at reflux for 2 h. Once the thioamide formation was completed, 2-chloro-3-oxopentanoic acid methyl ester (1.15 mL, 8.3 mmol) was added and the reaction mixture was stirred at reflux for an additional 5 h. The reaction was quenched with water and the solution was extracted with ether. The organic layers were combined and washed with HCl (1N, aq), NaHCO₃ (sat, aq) and brine. The organic layer was dried (Na₂SO₄), filtered and concentrated. The crude material was purified by flash chromatography on silica gel (5% ether in CH₂Cl₂) to afford 4-ethyl-2-isopropyl-thiazole-5-carboxylic acid methyl ester (920 mg, 52%).

To a solution of the above ester (207 mg, 0.97 mmol) in THF at 0° C. was added LAH (74 mg, 1.94 mmol). The reaction mixture was warmed to room temperature, stirred for 2 h and then quenched with water (75 μL), NaOH (15%, aq) (75 μL) and water (150 μL). The resulting suspension was stirred at room temperature for 15 min and the white solid was filtered off. The solution was dried (Na₂SO₄), filtered and concentrated to afford (4-ethyl-2-isopropylthiazol-5-yl)-methanol (190 mg). The crude material was used as is for the following step.

To a solution of the above alcohol (0.97 mmol) in dichloromethane (8 mL) was added 4-methylmorpholine-N-oxide (136 mg, 1.16 mmol) and TPAP (17 mg, 0.05 mmol). The reaction was stirred at room temperature for 2 h and the solvent was removed. The residue was purified by flash chromatography on silica gel column (5% ether in CH₂Cl₂) to afford 4-ethyl-2-isopropylthiazole-5-carbaldehyde (56 mg, 32%).

COMPOUND 43 was isolated as a white solid (21 mg, 36% over 2 steps). ¹H NMR (CDCl₃) δ 1.17 (t, 3H, J=7.5 Hz), 1.22-1.51 (m, 2H), 1.32 (d, 6H, J=7.2 Hz), 1.76 (d, 1H, J=11.0 Hz), 1.99-2.21 (m, 2H), 2.26 (t, 1H, J=11.8 Hz), 2.63 (q, 2H, J=7.5 Hz), 2.86-3.02 (m, 2H), 3.12 (d, 1H, J=10.6 Hz), 3.22 (hept, 1H, J=6.9 Hz), 3.54 (t, 1H, J=9.3 Hz), 3.74 (s, 2H), 3.75-3.88 (m, 1H), 4.44 (d, 1H, J=15.0 Hz), 4.52 (d, 1H, J=15.0 Hz), 4.53-4.63 (m, 1H), 6.05-6.75 (m, 1H), 7.09-7.25 (m, 4H), 7.36-7.51 (m, 2H), 7.91-7.99 (m, 2H); ES-MS m/z 547 (M+1).

### EXAMPLE 44

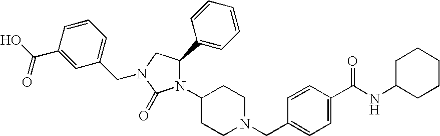

### COMPOUND 44: 3-{(R)-3-[1-(4-Cyclohexylcarbamoyl-benzyl)-piperidin-4-yl]-2-oxo-4-phenyl-imidazolidin-1-ylmethyl}-benzoic acid

Following general procedure G: a solution of 3-((R)-2-oxo-4-phenyl-3-piperidin-4-yl-imidazolidin-1-ylmethyl)-benzoic acid methyl ester (36 mg, 0.092 mmol), 4-bromomethyl-N-cyclohexyl-benzamide (see EXAMPLE 36) (27 mg, 0.091 mmol) and DIPEA (25 μL, 0.14 mmol) in CH₃CN (1.0 mL) was stirred at 55° C. for 17 hours. Standard work-up and purification gave 3-{(R)-3-[1-(4-cyclohexylcarbamoyl-benzyl)-piperidin-4-yl]-2-oxo-4-phenyl-imidazolidin-1-ylmethyl}-benzoic acid methyl ester as a white foam (38.0 mg, 69%).

Following general procedure H, a solution of the methyl ester (31 mg, 0.051 mmol) and 10M NaOH (0.10 mL, 1.0 mmol) in MeOH (1.0 mL) was stirred at 60° C. for 2.5 hours giving COMPOUND 44 as a white powder (28.3 mg, 93%). ¹H NMR (MeOH-d₄) δ 1.14-1.49 (m, 6H), 1.52-1.99 (m, 7H), 2.11-2.26 (m, 1H), 2.31-2.45 (m, 2H), 2.96-3.14 (m, 2H), 3.01 (dd, 1H, J=8.9, 7.1 Hz), 3.53-3.66 (m, 1H), 3.66 (t, 1H, J=9.4 Hz), 3.76-3.89 (m, 1H), 3.79 (s, 2H), 4.45 (s, 2H), 4.74 (dd, 1H, J=9.2, 7.0 Hz), 7.24-7.48 (m, 9H), 7.78 (d, 2H, J=8.4 Hz), 7.90 (d, 1H, J=7.5 Hz), 7.91 (s, 1H); ES-MS m/z 595 (M+H).

### EXAMPLE 45

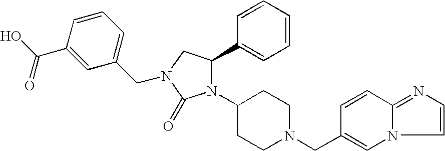

### COMPOUND 45: 3-[(R)-3-(1-Imidazo[1,2-a]pyridin-6-ylmethyl-piperidin-4-yl)-2-oxo-4-phenyl-imidazolidin-1-ylmethyl]-benzoic acid

Using general procedure A, 1,4-dioxa-8-aza-spiro[4.5]decane (76 μL, 0.59 mmol) and imidazo[1,2-a]pyridine-6-carbaldehyde (prepared according to literature procedure: Eisai Co., patent, U.S. Pat. No. 5,444,066 A1) (86 mg, 0.59 mmol) afforded 8-imidazo[1,2-a]pyridin-6-ylmethyl-1,4-dioxa-8-aza-spiro[4.5]decane (127 mg, 77%).

A solution of the above acetal (127 mg, 0.46 mmol) in a mixture of HCl (6N, aq)/acetone (1:1, 5 mL) and methanol (100 μL) was stirred at reflux for 3 h. The volatiles were removed under reduced pressure and the resulting aqueous solution was adjusted to pH˜7 with NaOH (1N, aq). The solution was extracted with CH₂Cl₂, dried over Na₂SO₄, filtered and concentrated. The crude 1-imidazo[1,2-a]pyridin-6-ylmethyl-piperidin-4-one (mixture of starting material/product 1:3) was used as is for the next step.

Using general procedure A, 3-[((R)-2-amino-2-phenylethylamino)-methyl]-benzoic acid methyl ester (50 mg, 0.18 mmol) and 1-imidazo[1,2-a]pyridin-6-ylmethyl-piperidin-4-one (54 mg, 0.24 mmol) afforded 3-{[(R)-2-(1-imidazo[1,2-a]pyridin-6-ylmethyl-piperidin-4-ylamino)-2-phenyl-ethylamino]-methyl}-benzoic acid methyl ester (42 mg, 48%).

Following general procedure K: to a cooled (0° C.) solution of the above diamine (42 mg, 0.08 mmol) and pyridine (13 μL, 0.16 mmol) in dry dichloromethane (3 mL) was slowly added triphosgene (13 mg, 0.04 mmol). The ice bath was removed and the mixture was stirred at ambient temperature for 2 h. Standard work-up and purification afforded 3-[(R)-3-(1-imidazo[1,2-a]pyridin-6-ylmethyl-piperidin-4-yl)-2-oxo-4-phenyl imidazolidin-1-ylmethyl]-benzoic acid methyl ester (19 mg, 45%).

Using general procedure H with the above methyl ester (19 mg, 0.04 mmol) gave COMPOUND 45 as a white solid (10 mg, 56%). ¹H NMR (CD₃OD) δ 0.87-2.33 (m, 10H), 2.93-3.11 (m, 3H), 3.51-3.74 (m, 4H), 4.49 (s, 2H), 3.79 (dd, 1H, J=9.3, 6.6 Hz), 7.13-7.65 (m, 9H), 7.75-7.98 (m, 4H), 7.96 (s, 1H); ES-MS m/z 510 (M+1).

### EXAMPLE 46

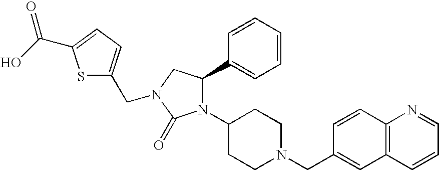

### COMPOUND 46: 5-[(R)-2-Oxo-4-phenyl-3-(1-quinolin-6-ylmethyl-piperidin-4-yl)-imidazolidin-1-ylmethyl]-thiophene-2-carboxylic acid

To a cooled (0° C.) solution of (2-hydroxy-1-phenyl-ethyl)-carbamic acid tert-butyl ester (34.59 g, 145.8 mmol), phthalimide (22.5 g, 153 mmol) and triphenylphosphine (42.1 g, 160 mmol) in dry THF (1 L) was added drop wise diethyl azodicarboxylate (24 mL, 153.1 mmol). The mixture was stirred for an additional 10 min. then warmed to ambient temperature, stirred for an additional 5 h, then concentrated under reduced pressure to afford [(R)-2-(1,3-dioxo-1,3-dihydroisoindol-2-yl)-1-phenylethyl]-carbamic acid tert-butyl ester as a white solid. A portion of this intermediate was use as is for the next step.

Using general procedure C, the above phthalimide (1.0 g, 2.7 mmol) afforded 2-((R)-2-amino-2-phenylethyl)-isoindole-1,3-dione (460 mg, 63%).

Using general procedure A, the above amine (324 mg, 1.22 mmol) and 1-boc-4-piperidone (245 mg, 1.22 mmol) afforded 4-[(R)-2-(1,3-dioxo-1,3-dihydroisoindol-2-yl)-1-phenylethylamino]-piperidine-1-carboxylic acid tert-butyl ester. The crude material was used as is for the following step.

To a solution of the above phthalimide (490 mg, 1.09 mmol) in ethanol (8 mL) was added hydrazine (530 μL, 10.6 mmol). The reaction mixture was stirred at room temperature for 4 h. The white precipitate was filtered and the filtrate was concentrated under reduced pressure. The crude material was purified by flash chromatography on silica gel (20% MeOH in CH₂Cl₂) to afford 4-((R)-2-amino-1-phenylethylamino)-piperidine-1-carboxylic acid tert-butyl ester (258 mg, 74%) as colorless oil.

A solution of the above amine (258 mg, 0.8 mmol) and 5-formylthiophene-2-carboxylic acid methyl ester (prepared according to literature procedure: Goddard, Carl J J. Heterocycl. Chem.; 1991; 28; 17-28.) (138 mg, 0.81 mmol) in MeOH (5 mL) was stirred at room temperature for 25 minutes. NaBH₄ (32 mg, 0.85 mmol) was added in one portion to the resulting suspension and the reaction was stirred for an additional 1 h. The solution was concentrated under reduced pressure, the residue was made basic (pH˜8) with 0.5M NaOH and was extracted with CH₂Cl₂. The combined organic solution was washed with brine, dried (Na₂SO₄), filtered and concentrated under reduced pressure. Purification by flash column chromatography on silica (CH₂Cl₂/MeOH, 8:1) gave 4-{(R)-2-[(5-methoxycarbonylthiophen-2-ylmethyl)-amino]-1-phenylethylamino}-piperidine-1-carboxylic acid tert-butyl ester (134 mg, 35%).

Following general procedure K: to a cooled (0° C.) solution of the above diamine (134 mg, 0.28 mmol) and pyridine (60 μL, 0.71 mmol) in dry CH₂Cl₂ (3 mL) was slowly added triphosgene (42 mg, 0.56 mmol). The ice bath was removed and the mixture was stirred at ambient temperature for 2 h. Standard work-up afforded crude 4-[(R)-3-(5-methoxycarbonylthiophen-2-ylmethyl)-2-oxo-5-phenylimidazolidin-1-yl]-piperidine-1-carboxylic acid tert-butyl ester. Using general procedure C, the above carbamate afforded 5-(®-2-oxo-4-phenyl-3-piperidin-4-yl-imidazolidin-1-ylmethyl)-thiophene-2-carboxylic acid methyl ester (105 mg, 93% over 2 steps).

Using general procedure A, the above amine (30 mg, 0.08 mmol) and quinoline-6-carbaldehyde (see EXAMPLE 35) (18 mg, 0.11 mmol) afforded 5-[(R)-2-oxo-4-phenyl-3-(1-quinolin-6-ylmethylpiperidin-4-yl)-imidazolidin-1-ylmethyl]-thiophene-2-carboxylic acid methyl ester (40 mg, 90%).

Using general procedure H, the above methyl ester (40 mg, 0.07 mmol) gave COMPOUND 46 (35 mg, 100%) as a white solid. ¹H NMR (CD₃OD) δ 1.85-2.02 (m, 3H), 2.38-2.58 (m, 1H), 3.04-3.22 (m, 3H), 3.40-3.74 (m, 3H), 3.78 (t, 1H, J=9.2 Hz), 4.50 (s, 2H), 4.56 (d, 1H, J=15.8 Hz), 4.67 (d, 1H, J=15.8 Hz), 4.82 (dd, 1H, J=9.6, 6.9 Hz), 7.04 (d, 1H, J=3.6 Hz), 7.31-7.43 (m, 5H), 7.61 (d, 1H, J=3.5 Hz), 7.67 (dd, 1H, J=8.4, 4.3 Hz), 7.91 (dd, 1H, J=8.3, 1.6 Hz), 8.11-8.20 (m, 2H), 8.49 (d, 1H, J=8.4 Hz), 8.97 (d, 1H, J=3.1 Hz); ES-MS m/z 527 (M+1).

### EXAMPLE 47

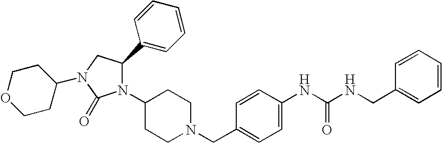

### COMPOUND 47: 1-Benzyl-3-(4-{4-[(R)-2-oxo-5-phenyl-3-(tetrahydro-pyran-4-yl)-imidazolidin-1-yl]-piperidin-1-ylmethyl}-phenyl)-urea

Following general procedure G, (R)-4-phenyl-3-piperidin-4-yl-1-(tetrahydro-pyran-4-yl)-imidazolidin-2-one (0.50 g, 1.5 mmol) was dissolved in CH₃CN (7.5 mL). Diisopropylethylamine (0.38 mL, 2.2 mmol) and 4-nitrobenzyl bromide (0.30 g, 1.4 mmol) were added to afford (R)-3-[1-(4-nitro-benzyl)-piperidin-4-yl]-4-phenyl-1-(tetrahydro-pyran-4-yl)-imidazolidin-2-one as a pale yellow oil (0.54 g, 76%).

The compound above (0.54 g, 1.2 mmol) was dissolved in ethanol (5 mL) and treated with tin (II) chloride dihydrate (1.05 g, 4.6 mmol) and concentrated HCl (17 mL), heating to 50° C. for 18 h. The reaction was cooled to 0° C., and 10N NaOH was added to basic pH. The solution was then extracted with CH₂Cl₂, dried (MgSO₄), and concentrated under reduced pressure to give (R)-3-[1-(4-amino-benzyl)-piperidin-4-yl]-4-phenyl-1-(tetrahydro-pyran-4-yl)-imidazolidin-2-one as a white solid (0.41 g, 80%). ¹H NMR (CDCl₃) δ 1.25 (m, 1H), 1.35 (m, 1H), 1.82 (br m, 5H), 1.75-1.96 (m, 3H), 2.70 (br d, 1H), 2.92 (br d, 1H), 3.03 (m, 1H), 3.29 (s, 2H), 3.47 (m, 2H), 3.62 (m, 4H), 3.97 (m, 3H), 4.58 (m, 1H), 6.59 (d, 2H, J=7.8 Hz), 7.00 (d, 2H, J=7.8 Hz), 7.30 (br m, 5H).

The above amine (50 mg, 0.11 mmol) was dissolved in isopropanol (0.5 mL) and benzylisocyanate (16 μL, 0.13 mmol) was added. The solution was stirred at room temperature for 16 hours and then concentrated under reduced pressure. The crude material was purified by flash column chromatography (50:1:1 CH₂Cl₂/MeOH/NH₄OH) on silica gel to afford COMPOUND 47 as a white solid (61 mg, 100%). ¹H NMR (CDCl₃) δ 1.25 (m, 1H), 1.36 (m, 1H), 1.65 (br m, 5H), 1.78-1.96 (m, 3H), 2.62 (br d, 1H), 2.80 (br d, 1H), 3.04 (m, 1H), 3.33 (s, 2H), 3.44 (m, 2H), 3.61 (m, 2H), 3.98 (m, 3H), 4.43 (d, 2H, J=5.7 Hz), 4.59 (m, 1H), 5.16 (m, 1H), 6.48 (s, 1H), 7.15 (m, 4H), 7.30 (br m, 10H); ES-MS m/z 568 (M+1).

### EXAMPLE 48

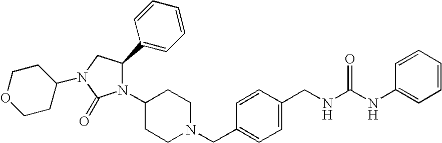

### COMPOUND 48: 1-(4-{4-[(R)-2-Oxo-5-phenyl-3-(tetrahydro-pyran4-yl)-imidazolidin-1-yl]-piperidin-1-ylmethyl}-benzyl)-3-phenyl-urea

Following general procedure G, (R)-4-phenyl-3-piperidin-4-yl-1-(tetrahydro-pyran-4-yl)-imidazolidin-2-one (0.50 g, 1.5 mmol) and 4-(bromomethyl)benzonitrile in CH₃CN afforded 4-{4-[(R)-2-oxo-5-phenyl-3-(tetrahydro-pyran-4-yl)-imidazolidin-1-yl]-piperidin-1-ylmethyl}-benzonitrile as a white solid (0.40 g, 66%).

The above nitrile (0.40 g, 0.9 mmol) was then dissolved in a saturated solution of ammonia in methanol (15 mL) in a Parr hydrogenator flask containing 1 scoop of Raney-Nickel® (pre-washed with ammonia/methanol solution). The reaction mixture was hydrogenated at 45 psi for 2 h, and then filtered through Celite® washing thoroughly with methanol. The filtrated was collected and concentrated under reduced pressure, affording ˜0.80 g of oil. Methanol (3 mL) and water (3 mL) were added and the solution treated with NaCN (0.25 g, 5.0 mmol) for 2 hours. The mixture was then partitioned between CH₂Cl₂, and brine and the organic phase dried (Na₂SO₄), filtered and concentrated under reduced pressure to afford (R)-3-[1-(4-aminomethyl-benzyl)-piperidin-4-yl]-4-phenyl-1-(tetrahydro-pyran-4-yl)-imidazolidin-2-one as a white solid (0.32 g, 80%). ¹H NMR (CDCl₃) δ 1.26 (m, 1H), 1.37 (m, 1H), 1.65 (br m, 5H), 1.85-2.03 (m, 3H), 2.70 (br d, 1H), 2.92 (br d, 1H), 3.05 (m, 1H), 3.40 (s, 2H), 3.45 (m, 2H), 3.66 (m, 2H), 3.83 (s, 2H), 3.99 (m, 3H), 4.59 (m, 1H), 7.21 (s, 4H), 7.32 (br s, 5H).

The above amine (35 mg, 78 μmol) was dissolved in isopropanol (0.5 mL) and phenylisocyanate (10 μL, 94 μmol) was added. The solution was stirred at room temperature for 16 hours and then concentrated under reduced pressure. The crude material was then purified by flash column chromatography (50:1:1, CH₂Cl₂/MeOH/NH₄OH) on silica gel to afford COMPOUND 48 as a white solid (28 mg, 66%). ¹H NMR (CDCl₃) δ 1.21 (m, 1H), 1.32 (m, 1H), 1.62 (br m, 5H), 1.72-1.96 (m, 3H), 2.64 (br d, 1H), 2.80 (br d, 1H), 3.05 (m, 1H), 3.36 (s, 2H), 3.60 (m, 1H), 3.65 (t, 1H, J=7.8 Hz), 3.96 (m, 3H), 4.37 (d, 2H, J=5.7 Hz), 4.59 (m, 1H), 5.41 (m, 1H), 6.94 (s, 1H), 7.03 (m, 1H), 7.14 (m, 4H), 7.30 (br m, 9H); ES-MS m/z 568 (M+H).

### EXAMPLE 49

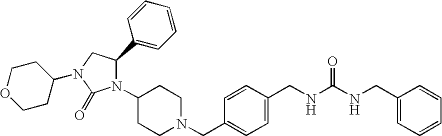

### COMPOUND 49: 1-Benzyl-3-(4-{4-[(R)-2-oxo-5-phenyl-3-(tetrahydro-pyran-4-yl)-imidazolidin-1-yl]-piperidin-1-ylmethyl}-benzyl)-urea

(R)-3-[1-(4-Aminomethyl-benzyl)-piperidin-4-yl]-4-phenyl-1-(tetrahydro-pyran-4-yl)-imidazolidin-2-one (see EXAMPLE 48) (35 mg, 78 μmol) was dissolved in isopropanol (0.5 mL) and benzylisocyanate (11 μL, 94 μmol) was added. The solution was stirred at room temperature for 16 hours and then concentrated under reduced pressure. The crude material was then purified by flash column chromatography (50:1:1 CH₂Cl₂/MeOH/NH₄OH) on silica gel to afford COMPOUND 49 as a white solid (30 mg, 67%). ¹H NMR (CDCl₃) δ 1.21 (m, 1H), 1.35 (m, 1H), 1.65 (br m, 5H), 1.75-1.99 (m, 3H), 2.64 (br d, 1H), 2.80 (br d, 1H), 3.05 (m, 1H), 3.37 (s, 2H), 3.44 (m, 2H), 3.65 (t, 1H, J=7.8 Hz), 3.65 (m, 1H), 3.98 (br m, 3H), 4.36 (m, 4H), 4.58 (m, 1H), 4.68 (m, 1H), 7.17 (s, 4H), 7.20-7.35 (m, 10H); ES-MS m/z 582 (M+H).

### EXAMPLE 50

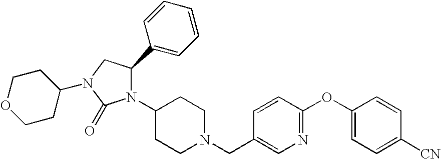

### COMPOUND 50: 4-(5-{4-[(R)-2-Oxo-5-phenyl-3-(tetrahydro-pyran-4-yl)-imidazolidin-1-yl]-piperidin-1-ylmethyl}-pyridin-2-yloxy)-benzonitrile

A mixture of 4-chlorophenol (12.0 g, 93.4 mmol), 2-bromo-5-methylpyridine (14.8 g, 86.0 mmol) and K₂CO₃ (20.7 g, 150 mmol) was stirred at 200° C. for 5 h. Aqueous work-up followed by purification by flash chromatography on silica gel (Et₂O/hexanes, 1:6 in v/v) afforded 2-(4-chloro-phenoxy)-5-methyl-pyridine as a colorless oil (14.1 g, 75%). ¹H NMR (CDCl₃) δ 2.28 (s, 3H), 6.83 (d, 1H, J=8.1 Hz), 7.02-7.08 (m, 2H), 7.26-7.36 (m, 2H), 7.49-7.53 (m, 1H), 7.99-8.01 (m, 1H).

Under N₂, to a dry flask charged with 2-(4-chloro-phenoxy)-5-methyl-pyridine (6.75 g, 30.8 mmol), Zn(CN)₂ (2.35 g, 20.0 mmol), Zn dust (0.400 g, 6.16 mmol), dppf (0.427 g, 0.770 mmol) and Pd₂(dba)₃ (0.284 g, 0.310 mmol) was added dry N,N-dimethylacetamide (40 mL). The mixture was stirred at 145° C. for 3 days and then cooled to room temperature. Aqueous ammonia (1 N, 50 mL) was added and the mixture was extracted with EtOAc (3×100 mL). The combined extract was washed with brine (100 mL) and dried over anhydrous Na₂SO₄. After filtration the solvent was removed, and the residue was purified by flash chromatography on silica gel (EtOAc/hexanes, 1:4 in v/v) to afford 4-(5-methyl-pyridin-2-yloxy)-benzonitrile as a pale yellow solid (3.15 g, 49%). ¹H NMR (CDCl₃) δ 2.32 (s, 3H), 6.91 (d, 1H, J=8.1 Hz), 7.16-7.22 (m, 2H), 7.58 (dd, 1H, J=2.4, 8.1 Hz), 7.64-7.68 (m, 2H), 8.04 (d, 1H, J=2.4 Hz).

A mixture of 4-(5-methyl-pyridin-2-yloxy)-benzonitrile (0.560 g, 2.69 mmol), NBS (0.958 g, 5.38 mmol) and benzoyl peroxide (0.100 g, 0.413 mmol) in CCl₄ (30 mL) was heated at reflux overnight. After the mixture was cooled to room temperature a solution of Na₂S₂O₃ (1 g) in water (20 mL) was added and the mixture was extracted with CH₂Cl₂ (2×30 mL). The combined organic extract was dried over anhydrous MgSO₄. After filtration the solvent was removed to afford a residue. The residue was dissolved in dry THF (10 mL), and diethyl phosphite (0.373 g, 2.70 mmol) and DIPEA (0.348 g, 2.70 mmol) was added. After the mixture was stirred at room temperature for 2 days, a saturated aqueous NaHCO₃ solution (15 mL) was added, and the mixture was extracted with EtOAc (2×20 mL). The combined extract was dried over anhydrous Na₂SO₄. After filtration the solvent was removed, and the residue was purified by flash chromatography on silica gel (EtOAc/hexanes, 1:4 in v/v) to afford 4-(5-bromomethyl-pyridin-2-yloxy)-benzonitrile as a pale yellow solid (0.410 g, 53%). ¹H NMR (CDCl₃) δ 4.47 (s, 3H), 7.01 (d, 1H, J=8.4 Hz), 7.22-7.27 (m, 2H), 7.67-7.72 (m, 2H), 7.81 (dd, 1H, J=8.4, 2.4 Hz), 8.18 (d, 1H, J=2.4 Hz).

Following General Procedure G: A mixture of (R)-4-phenyl-3-piperidin-4-yl-1-(tetrahydro-pyran-4-yl)-imidazolidin-2-one (0.329 g, 1.00 mmol), 4-(5-bromomethyl-pyridin-2-yloxy)-benzonitrile (0.287 g, 1.00 mmol) and DIPEA (0.193 g, 1.50 mmol) in CH₃CN (4 mL) was stirred at 70° C. overnight. Purification of the crude product by flash chromatography on silica gel (CH₂Cl₂/MeOH, 20:1 in v/v) gave COMPOUND 50 as a white foam (0.290 g, 54%). ¹H NMR (CDCl₃) δ 1.18-1.27 (m, 1H), 1.40-1.45 (m, 1H), 1.64-1.72 (m, 5H), 1.85-2.02 (m, 3H), 2.64-2.68 (m, 1H), 2.82-2.87 (m, 1H), 3.06 (dd, 1H, J=8.4, 6.9 Hz), 3.34-3.45 (m, 2H), 3.45-3.52 (m, 2H), 3.60-3.68 (m, 2H), 3.97-4.07 (m, 3H), 4.59 (dd, 1H, J=9.3, 6.9 Hz), 6.92 (d, 1H, J=8.4 Hz), 7.17-7.22 (m, 2H), 7.28-7.38 (m, 5H), 7.64-7.69 (m, 3H), 8.02 (d, 1H, J=2.4 Hz); ES-MS m/z 538 (M+H).

### EXAMPLE 51

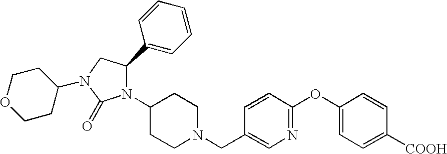

### COMPOUND 51: 4-(5-{4-[(R)-2-Oxo-5-phenyl-3-(tetrahydro-pyran-4-yl)-imidazolidin-1-yl]-piperidin-1-ylmethyl}-pyridin-2-yloxy)-benzoic acid

Following General Procedure 1: 4-(5-{4-[(R)-2-oxo-5-phenyl-3-(tetrahydro-pyran-4-yl)-imidazolidin-1-yl]-piperidin-1-ylmethyl}-pyridin-2-yloxy)-benzonitrile (COMPOUND 50) (0.100 g, 0.187 mmol) was suspended in a EtOH/10 N NaOH (5 mL, 3:2 in v/v). The mixture was stirred at 75° C. overnight. Purification of the crude product by flash chromatography on silica gel (CH₂Cl₂/MeOH, 10:1 in v/v) gave COMPOUND 51 as a pale yellow solid (0.079 g, 76%). ¹H NMR (CD₃OD) δ 1.61-1.87 (m, 7H), 2.33-2.40 (m, 1H), 2.90-3.05 (m, 2H), 3.13-3.18 (m, 1H), 3.38-3.58 (m, 5H), 3.75-3.82 (m, 1H), 3.87-4.00 (m, 3H), 4.24 (s, 2H), 4.70-4.86 (m, 1H), 7.12 (d, 1H, J=8.4 Hz), 7.19-7.22 (m, 2H), 7.30-7.41 (m, 51H), 7.93 (dd, 1H, J=8.4, 1.8 Hz), 8.05-8.10 (m, 2H), 8.20 (d, 1H, J=1.8 Hz); ¹³C NMR (CD₃OD) δ 28.69, 29.25, 31.40, 31.92, 50.86, 51.48, 54.11, 58.81, 68.74, 68.85, 114.25, 122.58, 128.91, 130.23, 130.89, 133.28, 143.49, 145.69, 152.35, 159.50, 165.54, 169.36; ES-MS m/z 557 (M+H). Anal. Calcd. for C₃₂H₃₆N₄O₅.1.6CH₂Cl₂.2.2H₂O: C, 55.12; H, 6.00; N, 7.65. Found: C, 55.07; H, 5.94; N, 7.90.

### EXAMPLE 52

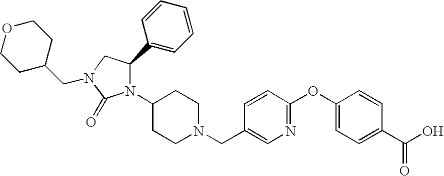

### COMPOUND 52: 4-(5-{4-[(R)-2-Oxo-5-phenyl-3-(tetrahydro-pyran-4-ylmethyl)-imidazolidin-1-yl]-piperidin-1-ylmethyl}-pyridin-2-yloxy)-benzoic acid

To a solution of methyltetrahydro-2-H-pyran-4-carboxylate (300 μL, 2.25 mmol) in methanol (500 μL) was added saturated aqueous 10N sodium hydroxide (250 μL). The mixture was heated at 60° C. for 3 h. The solution was concentrated under reduced pressure. Water (20 mL) was added and the pH of the solution was acidified to 1. The resulting acid was extracted with CH₂Cl₂ (5×20 mL) and the combined organic layers were dried over MgSO₄ and filtered. The filtrate was concentrated under reduced pressure and the crude tetrahydropyran-4-carboxylic acid was used in the next reaction without further purification.

Using general procedure F, tetrahydropyran-4-carboxylic acid (184 mg, 1.41 mmol) and (R)-(aminophenylmethyl)carbamic acid tert-butyl ester (368 mg, 1.55 mmol) afforded (R)-{phenyl-[(tetrahydropyran-4-carbonyl)amino]methyl}carbamic acid tert-butyl ester (432 mg, 80%).

Using general procedure C, the above substrate (432 mg, 1.24 mmol) afforded (R)-tetrahydropyran-4-carboxylic acid (aminophenylmethyl)amide (296 mg, 96%).

To a solution of (R)-tetrahydropyran-4-carboxylic acid (aminophenylmethyl)amide (296, 1.19) in dry THF was added BH₃.THF (4.8 mL of 1.0 M in THF, 4.77 mmol). This solution was stirred at 60° C. for 18 h. The reaction mixture was then cooled to room temperature. Methanol (3 mL) was added dropwise, then 6N HCl (2.4 mL) was added. The mixture was stirred at 60° C. for 1 h before THF was evaporated under reduced pressure. CH₂Cl₂ (40 mL) and saturated aqueous NaHCO₃ (40 mL) were added. The aqueous layer was extracted with CH₂Cl₂ (3×40 mL) and the combined organic extracts were dried over MgSO₄, filtered and concentrated under reduced pressure to afford (R)-C-phenyl-N-(tetrahydropyran-4-ylmethyl)methanediamine (239 mg, 86%), which was used in the next step without further purification.

Using general procedure A, the above diamine (239 mg, 1.02 mmol) and 1-boc-4-piperidone (223 mg, 1.12 mmol) afforded 4-({(R)-phenyl-[(tetrahydropyran-4-ylmethyl)amino]methyl}amino)piperidine-1-carboxylic acid tert-butyl ester (295 mg, 69%).

To a solution of the above substrate (295 mg, 0.705 mmol) and pyridine (90.0 μL, 1.06 mmol) in CH₂Cl₂ (7 mL) at 0° C. was added triphosgene (105 mg, 0.353 mmol). After 1 h, the reaction was quenched with saturated aqueous NaHCO₃ (20 mL) and after work-up and purification afforded 4-[(R)-2-oxo-5-phenyl-3-(tetrahydropyran-4-ylmethyl)imidazolidin-1-yl]piperidine-1-carboxylic acid tert-butyl ester (242 mg, 77%).

Using general procedure C, the above substrate (242 mg, 0.546 mmol) afforded (R)-4-phenyl-3-piperidin-4-yl-1-(tetrahydropyran-4-ylmethyl)-imidazolidin-2-one (188 mg, 99%).

Using general procedure G, (R)-4-phenyl-3-piperidin-4-yl-1-(tetrahydropyran-4-ylmethyl)-imidazolidin-2-one (42.7 mg, 0.124 mmol) and 4-(5-bromomethylpyridin-2-yloxy)benzonitrile (38.3 mg, 0.113 mmol) afforded 4-(5-{4-[(R)-2-oxo-5-phenyl-3-(tetrahydropyran-4-ylmethyl)imidazolidin-1-yl]piperidin-1-ylmethyl}pyridin-2-yloxy)benzonitrile (61.6 mg, 96%).

Using general procedure I, the above substrate (61.6 mg, 0.112 mmol) afforded COMPOUND 52 as beige solid (14.1 mg, 22%). ¹H NMR (CDCl₃) δ 1.17-1.67 (m, 4H), 1.70-1.87 (m, 3H), 2.11-2.44 (m, 3H), 2.94-3.19 (m, 4H), 3.34 (t, 2H, J=11.5 Hz), 3.35-3.66 (m, 3H), 3.67 (t, 1H, J=9.2 Hz), 3.81-4.01 (m, 3H), 4.61 (dd, 1H, J=8.7, 6.5 Hz), 6.85 (d, 1H, J=8.8 Hz), 7.09-7.32 (m, 7H), 7.68 (d, 1H, J=8.5 Hz), 8.00 (d, 2H, J=8.3 Hz), 8.06 (s, 1H); ¹³C NMR (CDCl₃) δ 27.5, 29.7, 30.1, 31.1, 34.4, 50.5, 51.2, 52.4, 52.7, 54.2, 56.2, 58.5, 68.0, 111.9, 120.9, 125.1, 127.1, 128.7, 129.0, 129.3 132.1, 142.3, 142.5, 149.8, 158.0, 161.4, 163.6, 169.5; ES-MS m/z 571 (M+H). Anal. Calcd. for C₃₃H₃₈N₄O₅.1.08H₂O.0.51CH₂Cl₂: C, 63.53; H, 6.55; N, 8.84. Found: C, 63.59; H, 6.64; N, 8.49.

### EXAMPLE 53

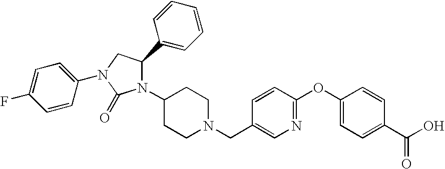

### COMPOUND 53: 4-(R)-(5-{4-[3-(4-Fluoro-phenyl)-2-oxo-5-phenyl-imidazolidin-1-yl]-piperidin-1-ylmethyl}-pyridin-2-yloxy)-benzoic acid

Following general procedure G: a solution of (R)-1-(4-fluoro-phenyl)-4-phenyl-3-piperidin-4-yl-imidazolidin-2-one (see EXAMPLE 77) (91.4 mg, 0.269 mmol), 4-(5-bromomethyl-pyridin-2-yloxy)-benzonitrile (70.8 mg, 0.245 mmol) and DIPEA (0.064 mL, 0.37 mmol) in CH₃CN (2.7 mL) was heated to 60° C. for 22 hours. Standard work-up and purification afforded 4-(5-{4-[(R)-3-(4-fluoro-phenyl)-2-oxo-5-phenyl-imidazolidin-1-yl]-piperidin-1-ylmethyl}-pyridin-2-yloxy)-benzonitrile (97 mg, 66%). Following general procedure I, the above nitrile (97 mg, 0.18 mmol) afforded COMPOUND 53 as a yellow solid (41 mg, 44%). ¹H NMR (CD₃OD) δ 1.88-1.99 (m, 3H), 2.37-2.49 (m, 1H), 2.93-3.01 (m, 2H), 3.23-3.52 (m, 4H), 3.61-3.75 (m, 2H), 4.17-4.29 (m, 3H), 7.04-7.11 (m, 3H), 7.21 (d, 2H, J=8.4 Hz), 7.23-7.56 (m, 7H), 7.99 (d, 1H, J=9.0 Hz), 8.08 (d, 2H, J=8.4 Hz), 8.20 (s, 1H); ¹³C NMR (CDCl₃) δ 28.2, 28.5, 51.7, 53.3, 54.3, 58.0, 58.2, 113.9, 116.6, 116.9, 121.8, 121.9, 122.3, 122.9, 128.7, 129.1, 130.3, 130.8, 133.1, 137.8, 142.8, 144.7, 151.7, 159.5, 159.7, 165.7, 169.6; ES-MS m/z 567 (M+1). Anal. Calcd. for C₃₃H₃₁N₄O₄F.0.81H₂O.1.03CH₂Cl₂: C, 61.10; H, 5.22; N, 8.37. Found: C, 61.13; H, 5.27; N, 8.20.

### EXAMPLE 54

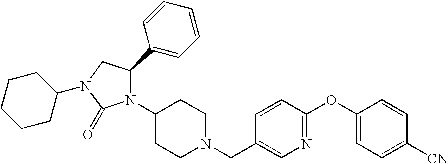

### COMPOUND 54: 4-{5-[4-((R)-3-Cyclohexyl-2-oxo-5-phenyl-imidazolidin-1-yl)-piperidin-1-ylmethyl]-pyridin-2-yloxy}-benzonitrile

Following general procedure G, (R)-1-cyclohexyl-4-phenyl-3-piperidin-4-yl-imidazolidin-2-one (0.16 g, 0.49 mmol) was dissolved in CH₃CN (3 mL). Diisopropylethylamine (0.13 mL, 0.74 mmol) and 4-(5-bromomethyl-pyridin-2-yloxy)-benzonitrile (0.15 g, 0.52 mmol) were then added, and the reaction stirred at 60° C. for 18 h. Standard work-up and purification afforded COMPOUND 54 as a white solid (0.17 g, 64%). ¹H NMR (CDCl₃) δ 1.00 (dq, 1H, J=10.8, 3.6 Hz), 1.15-1.45 (m, 6H), 1.65 (d, 2H, J=12.3 Hz), 1.75 (m, 4H), 1.84-2.05 (m, 3H), 2.67 (d, 1H, J=10.8 Hz), 2.83 (d, 1H, J=10.8 Hz), 3.05 (m, 1H), 3.38 (s, 2H), 3.62 (t, 1H, J=9.0 Hz), 3.64 (m, 1H), 4.55 (m, 1H), 6.92 (d, 1H, J=7.5 Hz), 7.20 (d, 2H, J=9.0 Hz), 7.33 (br s, 5H), 7.67 (d, 3H, J=7.8 Hz), 8.01 (s, 1H); ES-MS m/z 536 (M+H).

### EXAMPLE 55

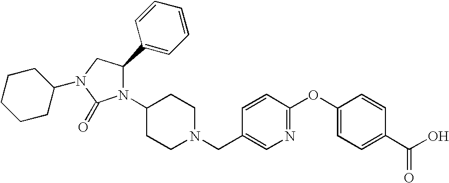

### COMPOUND 55: 4-{5-[4-((R)-3-Cyclohexyl-2-oxo-5-phenyl-imidazolidin-1-yl)-piperidin-1-ylmethyl]-pyridin-2-yloxy}-benzoic acid

Following general procedure I, 4-{5-[4-((R)-3-cyclohexyl-2-oxo-5-phenyl-imidazolidin-1-yl)-piperidin-1-ylmethyl]-pyridin-2-yloxy}-benzonitrile (COMPOUND 54) (77 mg, 0.14 mmol) was dissolved in ethanol (1 mL) and 10N NaOH (0.5 mL) to give COMPOUND 55 as a white solid (76 mg, 95%). ¹H NMR (CDCl₃) δ 1.04 (m, 1H), 1.20-1.51 (m, 7H), 1.63 (br m, 1H), 1.74 (br m, 4H), 2.12 (br t, 2H), 2.31 (br t, 1H), 2.97 (br d, 1H), 3.07 (t, 1H, J=7.5 Hz), 3.27 (br d, 1H), 3.47 (d, 1H, J=9.6 Hz), 3.65 (m, 2H), 3.79 (m, 2H), 4.55 (m, 1H), 6.79 (d, 1H, J=8.1 Hz), 7.12 (br m, 5H), 7.23 (br m, 2H), 7.57 (d, 1H, J=8.4 Hz), 7.97 (br d, 2H, J=8.7 Hz), 8.07 (s, 1H); ES-MS m/z 555 (M+H).

### EXAMPLE 56

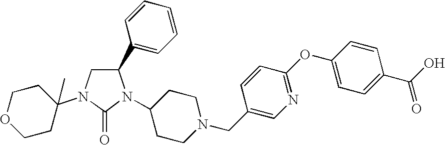

### COMPOUND 56: 4-(5-{4-[(R)-3-(4-Methyl-tetrahydro-pyran-4-yl)-2-oxo-5-phenyl-imidazolidin-1-yl]-piperidin-1-ylmethyl}-pyridin-2-yloxy)-benzoic acid

To a solution of methyl tetrahydro-2-H-pyran-4-carboxylate (2.00 g, 13.9 mmol) in THF (28 mL) at −78° C. was added NaHMDS (1.0M in THF, 20.8 mL, 20.8 mmol) slowly. MeI (2.60 mL, 41.7 mmol) was added and the mixture stirred at room temperature for 3 hours. Aqueous work-up afforded the crude ester (5.07 g) which was used with general procedure H to afford the crude acid (1.49 g). To a solution of the above acid (1.29 g) and Et₃N (1.31 mL, 9.40 mmol) in t-BuOH (9.9 mL) was added DPPA (2.12 mL, 9.84 mmol) and the mixture was heated at reflux for 8 hours. Standard work-up and purification afforded the carbamate (299 mg, 10% over 4 steps).

Following general procedure C, the above carbamate afforded 4-methyl-tetrahydro-pyran-4-ylamine (197 mg, 99%).

Following general procedure F: to a solution of (R)-tert-butoxycarbonylamino-phenyl-acetic acid (330 mg, 1.31 mmol) in THF (6.6 mL) at 0° C. was added NMM (133 mg, 1.31 mmol) in THF (0.5 mL) followed by IBCF (0.17 mL, 1.3 mmol) and the mixture stirred for 20 minutes. The above amine (197 mg, 1.71 mmol) was added and the mixture stirred at 0° C. for 30 min and at room temperature overnight. Standard work-up and purification gave [(R)-(4-methyl-tetrahydro-pyran-4-ylcarbamoyl)-phenyl-methyl]-carbamic acid tert-butyl ester (192 mg, 42%).

Following general procedure C with the above carbamate (192 mg, 0.551 mmol) gave the crude intermediate. Reduction with BH₃.THF (1.0M in THF, 2.2 mL, 2.2 mmol) in THF (5.4 mL) at reflux for 18 hours was followed by treatment with MeOH (3×) and subsequently 6N HCl (5 mL) at reflux for 1 hour. Basic work-up afforded (R)-N²-(4-methyl-tetrahydro-pyran-4-yl)-1-phenyl-ethane-1,2-diamine as a colourless oil (230 mg, 88% over 2 steps).

Following general procedure A, the above amine (114 mg, 0.102 mmol) and 1-BOC-4-piperidone (102 mg, 0.512 mmol) afforded the desired piperidine as a yellow solid (114 mg, 56%). Following general procedure K: to a solution of the piperidine (114 mg, 0.273 mmol) and Et₃N (0.076 mL, 0.55 mmol) in CH₂Cl₂ (5.5 mL) at 0° C. was added a solution of triphosgene (32 mg, 0.11 mmol) in CH₂Cl₂ (0.5 mL) and the mixture was stirred at room temperature for 2 hours. Standard work-up and purification afforded 4-{acetyl-[(R)-2-(4-methyl-tetrahydro-pyran-4-ylamino)-1-phenyl-ethyl]-amino}-piperidine-1-carboxylic acid tert-butyl ester (121 mg, quant). Following general procedure C, the above substrate (121 mg, 0.273 mmol) gave N-[(R)-2-(4-methyl-tetrahydro-pyran-4-ylamino)-1-phenyl-ethyl]-N-piperidin-4-yl-acetamide as a colourless oil (89 mg, 95%).

Following general procedure G: a solution of the above amine (52 mg, 0.15 mmol), 4-(5-bromomethyl-pyridin-2-yloxy)-benzonitrile (53 mg, 0.18 mmol) and DIPEA (0.042 mL, 0.24 mmol) in CH₃CN (1.0 mL) was heated to 60° C. for 20 hours. Standard work-up and purification afforded the nitrile (63 mg, 75%).

Following general procedure I, the above nitrile (63 mg, 0.11 mmol) afforded COMPOUND 56 as a yellow foam (65 mg, quant). ¹H NMR (CD₃OD) δ 1.30 (s, 3H), 1.58-1.99 (m, 5H), 2.21-2.38 (m, 3H), 2.88-3.00 (m, 2H), 3.10 (dd, 1H, J=9.0, 6.9 Hz), 3.31-3.36 (m, 1H), 3.43-3.47 (m, 1H), 3.54-3.77 (m, 6H), 4.20 (s, 2H), 4.69 (dd, 1H, J=9.0, 6.9 Hz), 7.12 (d, 1H, J=8.4 Hz), 7.19-7.22 (m, 2H), 7.33-7.45 (m, 5H), 7.92 (dd, 1H, J=8.4, 2.4 Hz), 8.06-8.09 (m, 2H), 8.19 (d, 1H, J=2.4 Hz); ¹³C NMR (CD₃OD) δ 22.31, 28.11, 28.87, 37:83, 37.99, 51.16, 51.45, 53.22, 53.30, 57.43, 58.01, 65.40, 65.53, 113.92, 122.29, 122.76, 128.53, 129.14, 130.07, 130.64, 133.11, 143.49, 144.86, 151.85, 159.49, 163.07, 165.63, 169.61; ES-MS m/z 571 (M+1). Anal. Calcd. for C₃₃H₃₈N₄O₅.1.1CH₂Cl₂: C, 61.67; H, 6.10; N, 8.44. Found: C, 61.53; H, 6.26; N, 8.42.

### EXAMPLE 57

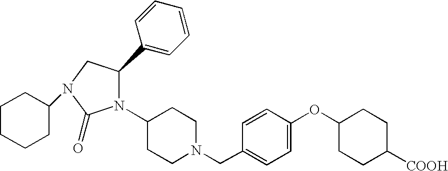

### COMPOUND 57: 4-{4-[4-((R)-3-Cyclohexyl-2-oxo-5-phenyl-imidazolidin-1-yl)-piperidin-1-ylmethyl]-phenoxy}-cyclohexanecarboxylic acid

To a solution of 4-hydroxy-cyclohexanecarboxylic acid ethyl ester (0.7 mL, 4.34 mmol), p-cresol (464.3 mg, 4.29 mmol) and triphenylphosphine (1.14 g, 4.34 mmol) in THF (25 mL) at 0° C. was added diisopropyl-azodicarboxylate (0.85 mL, 4.34 mmol) and the mixture was stirred for 18 hours at room temperature and quenched by adding a saturated NaHCO₃ solution. The aqueous layer was extracted three times with CH₂Cl₂, and the combined organic extracts were dried over MgSO₄ filtered and concentrated. The crude was purified by flash column chromatography on silica gel to give the desired product (700 mg, 69%).

A mixture of the above product (153 mg, 0.58 mmol), NBS (142 g, 0.58 mmol) and benzoyl peroxide (10 mg, 0.058 mmol) in CCl₄ (6 mL) was heated at reflux for one hour and quenched by adding a saturated NaHCO₃ solution. The aqueous layer was extracted three times with CH₂Cl₂, and the combined organic extracts were dried over MgSO₄, filtered and concentrated. The crude bromide (180 mg, 95%) was used in the next step without purification.

Following general procedure G and then H, the crude material above (180 mg, 0.55 mmol) and (R)-1-cyclohexyl-4-phenyl-3-piperidin-4-yl-imidazolidin-2-one (197 mg, 0.6 mmol) afforded COMPOUND 57 (100 mg, 59% in two steps). ¹H NMR (CDCl₃) δ 0.99-1.07 (m, 1H), 1.18-1.55 (m, 11H), 1.55-1.73 (m, 5H), 1.93-2.21 (m, 4H), 2.26-2.44 (m, 4H), 3.08 (dd, 2H, J=7.5, 7.5 Hz), 3.35 (d, 1H, J=11.1 Hz), 3.62-3.92 (m, 6H), 4.14-4.60 (m, 1H), 4.61 (dd, 1H, J=9, 6 Hz), 6.85 (dd, 2H, J=7.5, 7.5 Hz), 7.26-7.33 (m, 7H); ¹³C NMR (CDCl₃) δ 23.92, 25.48, 25.54, 26.99, 28.96, 30.04, 30.26, 30.81, 41.75, 42.52, 48.39, 49.64, 51.19, 51.45, 53.44, 55.62, 59.37, 115.93, 116.24, 126.70, 128.33, 129.02, 132.50, 142.52, 158.50, 159.87, 179.03; ES-MS m/z 560 (M+H).

### EXAMPLE 58

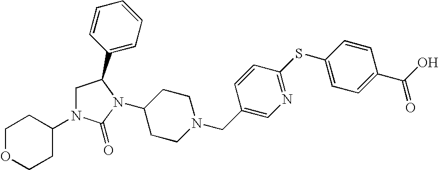

### COMPOUND 58: 4-(5-{4-[(R)-2-Oxo-5-phenyl-3-(tetrahydro-pyran-4-yl)-imidazolidin-1-yl]-piperidin-1-ylmethyl}-pyridin-2-ylsulfanyl)-benzoic acid

Following general procedure G: to a solution of the (R)-4-phenyl-3-piperidin-4-yl-1-(tetrahydropyran-4-yl)-imidazolidin-2-one (80 mg, 0.24 mmol) dissolved in acetonitrile (2.2 mL) was added DIPEA (61 μL, 0.331 mmol) followed by 4-(5-bromomethyl-pyridin-2-ylsulfanyl)-benzoic acid methyl ester (75 mg) and the mixture was heated to 75° C. for 4.5 hours. Standard work-up and purification afforded 4-(5-{4-[(R)-2-oxo-5-phenyl-3-(tetrahydro-pyran-4-yl)-imidazolidin-1-yl]-piperidin-1-ylmethyl}-pyridin-2-ylsulfanyl)-benzoic acid methyl ester as a white foam (105 mg, 81%).

Following general procedure H, the ester prepared above (105 mg, 0.18 mmol) afforded COMPOUND 58 as a white precipitate (64 mg, 62%). ¹H NMR (CDCl₃) δ 1.48 (br s, 1H), 1.64-1.74 (m, 4H), 1.86-1.90 (m, 1H), 2.53 (br s, 2H), 2.72 (br s, 1H), 3.12-3.21 (m, 2H), 3.43-3.52 (m, 4H), 3.70 (t, 1H, J=9.3 Hz), 3.83-4.20 (m, 6H), 4.61-4.65 (m, 1H), 6.98 (d, 1H, J=6.0 Hz), 7.19-7.31 (m, 6H), 7.60 (d, 2H, J=8.1 Hz), 8.01 (d, 2H, J=8.1 Hz), 8.30 (s, 1H); ¹³C NMR (CDCl₃) δ 25.7, 27.3, 29.8, 30.1, 48.3, 48.9, 49.3, 51.5, 53.5, 55.7, 67.1, 67.2, 122.0, 126.7, 128.6, 129.2, 131.0, 132.1, 134.4, 135.4, 140.0, 141.8, 151.1, 159.7, 162.5, 168.5; ES-MS m/z 573 (M+H). Anal. Calcd. for C₃₂H₃₆N₄O₄S.1.6CH₂Cl₂: C, 56.95; H, 5.58; N, 7.91. Found: C, 56.91; H, 5.90; N, 8.00.

### EXAMPLE 59

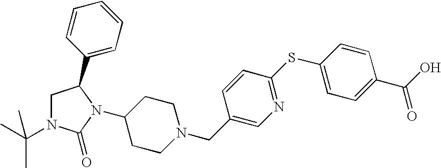

### COMPOUND 59: 4-{5-[4-((R)-3-tert-Butyl-2-oxo-5-phenyl-imidazolidin-1-yl)-piperidin-1-ylmethyl]-pyridin-2-ylsulfanyl}-benzoic acid

Following general procedure G: to a solution of (R)-1-tert-butyl-4-phenyl-3-piperidin-4-yl-imidazolidin-2-one (67 mg, 0.22 mmol) in acetonitrile (2 mL) was added DIPEA (0.055 mL, 0.30 mmol) followed by 4-(5-bromomethyl-pyridin-2-ylsulfanyl)-benzoic acid methyl ester (68 mg, 0.20 mmol) and the mixture was heated to 75° C. for 3 hours. Standard work-up and purification afforded 4-{5-[4-((R)-3-tert-butyl-2-oxo-5-phenyl-imidazolidin-1-yl)-piperidin-1-ylmethyl]-pyridin-2-ylsulfanyl}-benzoic acid methyl ester as a white foam (49 mg, 44%).

Following general procedure H, the ester prepared above (49 mg, 0.088 mmol) afforded COMPOUND 59 as a white solid (18 mg, 38%). ¹H NMR (CDCl₃) δ 1.35 (s, 9H), 1.34-1.45 (m, 1H), 1.74 (br s, 1H), 1.88 (d, 1H, J=12.3 Hz) 2.16 (s, 1H), 2.54-2.64 (m, 2H), 2.81-2.89 (m, 1H), 3.14-3.22 (m, 2H), 3.52 (d, 1H, J=8.7), 3.71 (t, 1H, J=9.0 Hz), 3.97-4.16 (m, 2H), 4.51-4.53 (m, 1H), 7.01 (d, 1H, J=8.1 Hz), 7.23-7.36 (m, 5H), 7.57 (d, 2H, J=8.1 Hz), 7.99 (d, 2H, J=8.4 Hz), 8.10 (d, 1H, J=7.5 Hz), 8.34 (s, 1H); ¹³C NMR (CDCl₃) δ 25.8, 27.9, 49.4, 51.1, 52.5, 53.9, 55.1, 122.0, 122.6, 127.2, 128.9, 129.5, 131.3, 131.8, 134.6, 136.2, 140.6, 142.3, 151.6, 160.9, 162.8, 168.7; ES-MS m/z 545 (M+H). Anal. Calcd. for C₃₁H₃₆N₄O₃S.1.7CH₂Cl₂: C, 57.00; H, 5.76; N, 8.13. Found: C, 57.20; H, 5.95; N, 8.16.

Examples 60 to 72 were prepared following the scheme illustrated below. RCHO is as defined in the table and X is as defined in the individual examples.

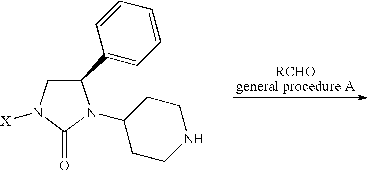

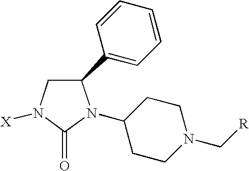

| Exam- | |
|---|---|
| ple | RCHO |
| 60 | 4-(4-formyl-phenoxy)-benzoic acid |
| 61 | 6-(6-chloro-pyridin-3-yloxy)-pyridine-3-carbaldehyde |
| 62 | 2-methyl-6-(1-oxo-2,3-dihydro-1H-isoindol-5-yloxy)-pyridine-3- |
| | carbaldehyde |
| 63 | 6-(benzo[1,3]dioxol-5-yloxy)-2-methyl-pyridine-3-carbaldehyde |
| | (see EXAMPLE 276) |
| 64 | 6-(4-methoxy-phenylsulfanyl)-2-methyl-pyridine-3-carbaldehyde |
| 65 | N-cyclopropyl-4-(5-formyl-6-methyl-pyridin-2-yloxy)- |
| | benzamide |
| 66 | N-cyclopropyl-4-(5-formyl-6-methyl-pyridin-2-yloxy)- |
| | benzamide |
| 67 | N-cyclopropyl-4-(5-formyl-6-methyl-pyridin-2-yloxy)- |
| | benzamide |
| 68 | 4-(5-formyl-pyridin-2-ylsulfanyl)-benzoic acid |
| 69 | 4-(5-formyl-pyridin-2-ylsulfanyl)-benzoic acid |
| 70 | 4-(5-formyl-pyridin-2-ylsulfanyl)-benzoic acid |
| 71 | N-cyclopropyl-4-(5-formyl-6-methyl-pyridin-2-yloxy)- |
| | benzamide |
| 72 | 6-(4-methoxy-phenoxy)-2-methyl-pyridine-3-carbaldehyde |

### EXAMPLE 60

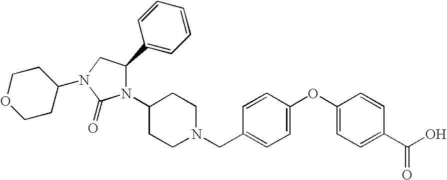

### COMPOUND 60: 4-(4-{4-[(R)-2-Oxo-5-phenyl-3-(tetrahydro-pyran-4-yl)-imidazolidin-1-yl]-piperidin-1-ylmethyl}-phenoxy)-benzoic acid

COMPOUND 60 was isolated as a white powder (28.8 mg, 39%). ¹H NMR (CDCl₃) δ 1.47-1.83 (m, 7H), 2.18 (qd, 1H, J=12.7, 3.5 Hz), 2.37-2.51 (m, 2H), 3.02-3.22 (m, 3H), 3.43-3.53 (m, 2H), 3.59 (tt, 1H, J=11.8, 4.0 Hz), 3.74-3.81 (m, 1H), 3.78 (s, 2H), 3.88-4.02 (m, 3H), 4.74 (dd, 1H, J=9.2, 7.0 Hz), 6.96 (d, 2H, J=8.3 Hz), 7.01 (d, 2H, J=8.3 Hz), 7.28-7.41 (m, 7H), 7.97 (d, 2H, J=8.5 Hz); ¹³C NMR (CDCl₃) δ 28.58, 29.80, 30.80, 31.16, 49.53, 50.45, 52.26, 53.21, 53.44, 57.91, 61.60, 68.16, 68.25, 118.69, 120.43, 128.04, 129.54, 129.76, 130.11, 131.44, 132.65, 133.38, 143.35, 158.34, 161.18, 161.77, 170.93; ES-MS m/z 556 (M+1). Anal. Calcd. for C₃₃H₃₇N₃O₅.0.7CH₂Cl₂: C, 65.80; H, 6.29; N, 6.83. Found: C, 65.88; H, 6.40; N, 6.82.

### EXAMPLE 61

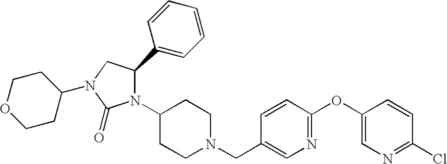

### COMPOUND 61: (R)-3-{1-[6-(6-Chloro-pyridin-3-yloxy)-pyridin-3-ylmethyl]-piperidin-4-yl}-4-phenyl-1-(tetrahydro-pyran-4-yl)-imidazolidin-2-one

A mixture of 6-bromo-pyridine-3-carbaldehyde (0.930 g, 5.00 mmol), 6-chloropyridin-3-ol (0.650 g, 5.00 mmol) and K₂CO₃ (0.690 g, 5.00 mmol) in DMF (10 mL) was stirred at 130° C. for 2 h. The mixture was cooled to room temperature, DMF was removed and water (30 mL) was added. The mixture was extracted with CH₂Cl₂ (3×30 mL) and the combined extract was dried over anhydrous Na₂SO₄. After filtration the solvent was removed, and the residue was purified by flash chromatography on silica gel (Et₂O/CH₂Cl₂, 1:20 v/v) to afford 6-(6-chloro-pyridin-3-yloxy)-pyridine-3-carbaldehyde as a white solid (1.15 g, 98%). ¹H NMR (CDCl₃) δ 7.14 (d, 1H, J=8.7 Hz), 7.41 (d, 1H, J=8.7 Hz), 7.55 (dd 1H, J=8.7, 3.0 Hz), 8.24 (dd, 1H, J=8.7, 2.1 Hz), 8.31 (d, 1H, J=3.0 Hz), 8.58 (d, 1H, J=2.1 Hz).

COMPOUND 61 was isolated as a white foam (0.088 g, 70%). ¹H NMR (CDCl₃) δ 1.16-1.25 (m, 1H), 1.38-1.44 (m, 1H), 1.63-1.71 (m, 5H), 1.84-2.04 (m, 3H), 2.64-2.67 (m, 1H), 2.82-2.85 (m, 1H), 3.05 (dd, 1H, J=8.4, 6.9 Hz), 3.36 (s, 2H), 3.44-3.52 (m, 2H), 3.61-3.68 (m, 2H), 3.97-4.05 (m, 3H), 4.58 (dd, 1H, J=9.3, 6.3 Hz), 6.91 (d, 1H, J=8.4 Hz), 7.30-7.37 (m, 6H), 7.48 (dd, 1H, J=8.7, 2.7 Hz), 7.64 (dd, 1H, J=8.4, 2.1 Hz), 7.94 (d, 1H, J=2.1 Hz), 8.25 (d, 1H, J=2.7 Hz); ¹³C NMR (CDCl₃) δ 29.20, 30.01, 30.39, 31.00, 48.62, 48.90, 52.28, 53.15, 53.29, 56.34, 59.30, 67.40, 67.49, 111.50, 124.77, 126.87, 128.44, 129.06, 129.87, 132.01, 141.03, 142.86, 143.21, 146.43, 147.46, 149.92, 160.25, 161.84. ES-MS m/z 605 (M+H). Anal. Calcd. for C₃₀H₃₄ClN₅O₃.0.15CH₂Cl₂: C, 64.57; H, 6.16; N, 12.49; Cl, 8.22. Found: C, 64.59; H, 6.15; N, 12.38; Cl, 7.99.

### EXAMPLE 62

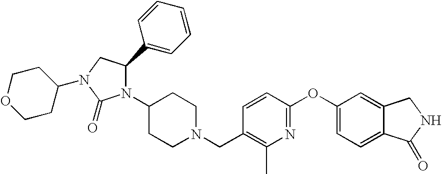

### COMPOUND 62: 5-(6-Methyl-5-{4-[(R)-2-oxo-5-phenyl-3-(tetrahydro-pyran-4-yl)-imidazolidin-1-yl]-piperidin-1-ylmethyl}-pyridin-2-yloxy)-2,3-dihydro-isoindol-1-one

A suspension of 2-methyl-4-methoxy-benzoic acid (3.32 g, 20.0 mmol) and concentrated H₂SO₄ (1 mL) in methanol (20 mL) was heated at reflux for 5 h. Standard work-up and purification by flash chromatography on silica gel (EtOAc/hexanes, 1:2 in v/v) afforded 2-methyl-4-methoxy-benzoic acid methyl ester as a liquid (3.38 g, 94%).

A mixture of 2-methyl-4-methoxy-benzoic acid methyl ester (3.38 g, 18.8 mmol), NBS (5.02 g, 28.2 mmol) and benzoylperoxide (0.910 g, 3.76 mmol) in CCl₄ (50 mL) was heated at reflux for 16 h. The mixture was cooled to room temperature and saturated aqueous NaHCO₃ (30 mL) was added. Extraction with CH₂Cl₂ (3×50 mL) was performed and the extract was dried over anhydrous Na₂SO₄. After filtration the solvent was removed, and the residue was dissolved in methanol (40 mL). The solution was saturated with NH₃ gas, and then heated at 60° C. for 2 h. Aqueous work-up and purification by flash chromatography on silica gel (EtOAc) afforded 5-methoxy-2,3-dihydro-isoindol-1-one as a white solid (0.830 g, 27%).

To a solution of 5-methoxy-2,3-dihydro-isoindol-1-one (0.830 g, 5.09 mmol) in CH₂Cl₂ (75 mL) was added BBr₃ (1.0 M in CH₂Cl₂, 15.3 mL, 15.3 mmol). The mixture was stirred at room temperature for 16 h, forming a suspension. Methanol (30 mL) was added and the mixture was stirred for 10 min. The solvent was then removed and the residue was washed with water to afforded 5-hydroxy-2,3-dihydro-isoindol-1-one as an off-white solid (0.717 g, 95%).

A mixture of 6-bromo-2-methylpyridine-3-carboxaldehyde (0.350 g, 1.75 mmol), 5-hydroxy-2,3-dihydro-isoindol-1-one (0.223 g, 1.50 mmol) and K₂CO₃ (0.124 g, 0.900 mmol) in DMF (5 mL) was stirred at 115° C. for 1.5 h. The mixture was cooled to room temperature and DMF was removed. Aqueous work-up and purification by flash chromatography on silica gel (CH₂Cl₂/CH₃OH, 20:1 in v/v) afforded 2-methyl-6-(1-oxo-2,3-dihydro-1H-isoindol-5-yloxy)-pyridine-3-carbaldehyde as a pale yellow solid (0.10 g, 20%). ¹H NMR (CDCl₃) δ 2.71 (s, 3H), 4.49 (s, 2H), 6.87 (d, 1H, J=8.4 Hz), 3H), 7.24-7.28 (m, 2H), 7.70 (br s, 1H), 7.89 (d, 1H, J=8.1 Hz), 8.13 (d, 1H, J=8.4 Hz), 10.24 (s, 1H).

COMPOUND 62 was isolated as a pale yellow solid (0.103 g, 54%). ¹H NMR (CDCl₃) δ 1.18-1.26 (m, 1H), 1.41-1.44 (m, 1H), 1.60-1.70 (m, 5H), 1.88-2.06 (m, 3H), 2.38 (s, 3H), 2.64-2.67 (m, 1H), 2.81-2.85 (m, 1H), 3.06 (dd, 1H, J=8.4, 6.9 Hz), 3.32-3.38 (m, 2H), 3.44-3.52 (m, 2H), 3.62-3.68 (m, 2H), 3.98-4.04 (m, 3H), 4.42 (s, 2H), 4.58 (dd, 1H, J=9.3, 6.3 Hz), 6.63-6.66 (m, 3H), 7.15-7.18 (m, 2H), 7.28-7.37 (m, 5H), 7.51 (d, 1H, J=8.4 Hz), 7.82-7.85 (m, 1H); ¹³C NMR (CDCl₃) δ 22.03, 29.32, 29.99, 30.37, 30.91, 45.64, 48.57, 48.89, 52.39, 53.36, 53.45, 56.40, 59.06, 67.37, 67.47, 108.86, 114.68, 120.45, 125.20, 126.87, 127.78, 128.09, 128.41, 129.02, 141.23, 142.79, 145.78, 156.86, 158.63, 160.23, 160.95, 171.58; ES-MS m/z 582 (M+H). Anal. Calcd. for C₃₄H₃₉N₅O₄.0.6CH₂Cl₂: C, 65.69; H, 6.40; N, 11.07. Found: C, 65.64; H, 6.43; N, 11.05.

### EXAMPLE 63

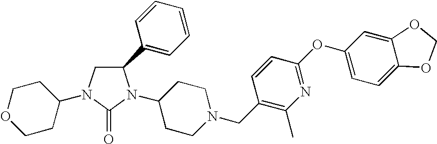

### COMPOUND 63: (R)-3-{1-[6-(Benzo[1,3]dioxol-5-yloxy)-2-methyl-pyridin-3-ylmethyl]-piperidin-4-yl}-4-phenyl-1-(tetrahydro-pyran-4-yl)-imidazolidin-2-one

COMPOUND 63 was isolated as a white solid (82 mg, 64%). ¹H NMR (CDCl₃) δ 1.13-1.24 (m, 1H), 1.37-1.42 (m, 1H), 1.62-1.68 (m, 5H), 1.81-2.04 (m, 3H), 2.38 (s, 3H), 2.61-2.64 (m, 1H), 2.79-2.82 (m, 1H), 3.06-3.30 (m, 1H), 3.28 (s, 2H), 3.43-3.51 (m, 2H), 3.61-3.67 (m, 2H), 3.97-4.06 (m, 3H), 4.58 (dd, 1H, J=8.7, 6.9 Hz), 5.96 (s, 2H), 6.45 (d, 1H, J=8.1 Hz), 6.55 (dd, 1H, J=8.4, 1.8 Hz), 6.63 (d, 1H, J=1.8 Hz), 6.76 (d, 1H, J=8.4 Hz), 7.26-7.41 (m, 3H); ¹³C NMR (CDCl₃) δ 22.28, 29.51, 30.21, 30.58, 31.23, 48.78, 49.10, 52.61, 53.47, 53.63, 56.54, 59.31, 67.59, 67.69, 101.91, 103.74, 107.05, 108.57, 113.70, 126.99, 127.09, 128.62, 129.23, 141.18, 143.06, 144.68, 148.55, 149.42, 156.90, 160.43, 162.65; ES-MS m/z 571 (M+1). Anal. Calcd. for C₃₃H₃₈N₄O₅.0.58CH₄O.0.09CH₂Cl₂: C, 67.79; H, 6.84; N, 9.39. Found: C, 67.83; H, 6.75; N, 9.21.

### EXAMPLE 64

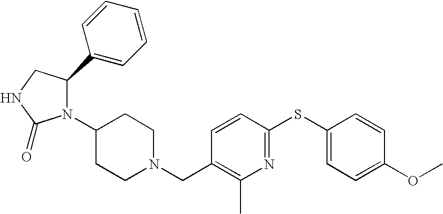

### COMPOUND 64: (R)-1-{1-[6-(4-Methoxy-phenylsulfanyl)-2-methyl-pyridin-3-ylmethyl]-piperidin-4-yl}-5-phenyl-imidazolidin-2-one

COMPOUND 64 was isolated as a white powder (286 mg, 67%). ¹H NMR (CDCl₃) δ 1.05-1.19 (m, 1H), 1.38 (d, 1H, J=12.3 Hz), 1.64 (d, 1H, J=12.3 Hz), 1.79-2.02 (m, 3H), 2.43 (s, 3H), 2.59 (d, 1H, J=11.4 Hz), 2.77 (d, 1H, J=10.8 Hz), 3.21 (t, 1H, J=7.2 Hz), 3.24 (s, 2H), 3.64 (tt, 1H, J=12.0, 3.6 Hz), 3.74 (t, 1H, J=9.0 Hz), 3.85 (s, 3H), 4.48 (br s, 1H), 4.71 (dd, 1H, J=9.0, 6.6 Hz), 6.45 (d, 1H, J=8.1 Hz), 6.94 (d, 2H, J=8.7 Hz), 7.18 (d, 1H, J=8.1 Hz), 7.29-7.36 (m, 5H), 7.51 (d, 2H, J=8.7 Hz); ¹³C NMR (CDCl₃) δ 22.4, 29.6, 31.4, 48.6, 52.2, 53.4, 53.6, 55.8, 58.9, 59.6, 115.6, 117.7, 122.0, 127.2, 128.2, 128.6, 129.2, 137.6, 138.3, 142.9, 158.1, 160.5, 160.9, 163.0; ES-MS m/z 489 (M+H). Anal. Calcd. for C₂₈H₃₂N₄O₂S.0.2CH₂Cl₂: C, 66.99; H, 6.46; N, 11.08. Found: C, 66.60; H, 6.44; N, 10.86.

### EXAMPLE 65

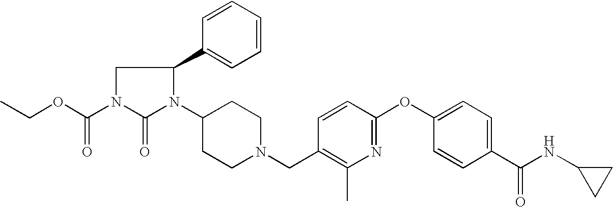

### COMPOUND 65: (R)-3-{1-[6-(4-Cyclopropylcarbamoyl-phenoxy)-2-methyl-pyridin-3-ylmethyl]-piperidin-4-yl}-2-oxo-4-phenyl-imidazolidine-1-carboxylic acid ethyl ester

COMPOUND 65 was isolated as a white solid (139 mg, 74%). ¹H NMR (CDCl₃) δ 0.58-0.63 (m, 2H), 0.82-0.88 (m, 2H), 1.31-1.36 (m, 1H), 1.33 (t, 3H, J=7.2 Hz), 1.45 (d, 1H, J=12.0 Hz), 1.66 (d, 1H, J=9.3 Hz), 1.91-1.99 (m, 2H), 2.35 (s, 3H), 2.66 (d, 1H, J=10.5 Hz), 2.81-2.90 (m, 2H), 3.31 (s, 2H), 3.60 (dd, 1H, J=10.5, 5.1 Hz), 3.62 (m, 1H), 4.11 (t, 1H, J=10.2 Hz), 4.28 (q, 2H, J=7.2 Hz), 4.62 (dd, 1H, J=9.3, 4.8 Hz), 6.33 (br s, 1H), 6.59 (d, 1H, J=8.1 Hz), 7.09 (d, 2H, J=8.7 Hz), 7.27-7.40 (m, 5H), 7.48 (d, 1H, J=8.1 Hz), 7.73 (d, 2H, J=8.7 Hz); ¹³C NMR (CDCl₃) δ 7.09, 14.75, 22.18, 23.53, 29.52, 30.48, 50.73, 52.93, 53.28, 53.35, 55.17, 59.18, 62.93, 108.80, 120.31, 126.83, 127.89, 128.98, 129.10, 129.51, 130.34, 141.39, 141.78, 152.45, 154.01, 156.99, 157.99, 161.23, 168.69; ES-MS m/z 598 (M+H). Anal. Calcd. for C₃₄H₃₉N₅O₅.0.2CH₂Cl₂: C, 66.83; H, 6.46; N, 11.39. Found: C, 66.69; H, 6.54; N, 11.04.

### EXAMPLE 66

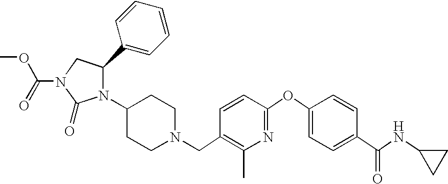

### COMPOUND 66: (R)-3-{1-[6-(4-Cyclopropylcarbamoyl-phenoxy)-2-methyl-pyridin-3-ylmethyl]-piperidin-4-yl}-2-oxo-4-phenyl-imidazolidine-1-carboxylic acid methyl ester

COMPOUND 66 was isolated as a white powder (31.4 mg, 33%). ¹H NMR (CDCl₃) δ 0.58-0.64 (m, 2H), 0.83-0.89 (m, 2H), 1.23-1.35 (m, 2H), 1.45 (d, 1H, J=11.1 Hz), 1.66 (d, 1H, J=9.3 Hz), 1.88-2.04 (m, 3H), 2.36 (s, 3H), 2.67 (d, 1H, J=11.4 Hz), 2.81-2.92 (m, 2H), 3.32 (s, 2H), 3.62 (dd, 1H, J=10.8, 4.8 Hz), 3.63 (m, 1H), 3.84 (s, 3H), 4.11 (t, 1H, J=9.9 Hz), 4.63 (dd, 1H, J=9.6, 4.8 Hz), 6.27 (s, 1H), 6.59 (d, 1H, J=8.1 Hz), 7.10 (d, 2H, J=8.4 Hz), 7.25-7.30 (m, 2H), 7.32-7.39 (m, 3H), 7.48 (d, 1H, J=8.1 Hz), 7.73 (d, 2H, J=8.4 Hz); ¹³C NMR (CDCl₃) δ 7.1, 22.2, 23.5, 29.5, 30.4, 50.8, 52.9, 53.2, 53.3, 53.8, 55.2, 59.1, 108.8, 120.4, 126.8, 127.8, 129.0, 129.2, 129.6, 130.3, 141.5, 141.7, 153.0, 153.9, 157.0, 158.0, 161.3, 168.7; ES-MS m/z 584 (M+H). Anal. Calcd. for C₃₃H₃₇N₅O₅.0.3CH₂Cl₂: C, 65.66; H, 6.22; N, 11.50. Found: C, 65.89; H, 6.34; N, 11.23.

### EXAMPLE 67

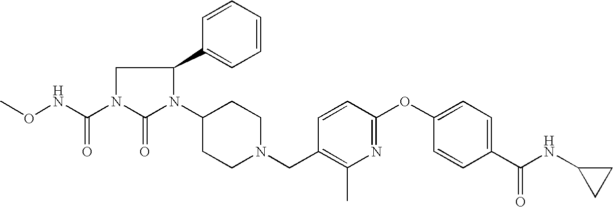

### COMPOUND 67: (R)-3-{1-[6-(4-Cyclopropylcarbamoyl-phenoxy)-2-methyl-pyridin-3-ylmethyl]-piperidin-4-yl}-2-oxo-4-phenyl-imidazolidine-1-carboxylic acid methoxy-amide

COMPOUND 67 was isolated as a white solid (25.5 mg, 43%). ¹H NMR (CDCl₃) δ 0.58-0.63 (m, 2H), 0.82-0.88 (m, 2H), 1.24-1.39 (m, 1H), 1.44 (d, 1H, J=11.1 Hz), 1.63 (m, 1H), 1.86-1.99 (m, 3H), 2.36 (s, 3H), 2.68 (d, 1H, J=10.2 Hz), 2.82-2.90 (m, 2H), 3.31 (s, 2H), 3.51 (m, 1H), 3.66 (dd, 1H, J=10.8,5.4 Hz), 4.89 (s, 3H), 4.14 (t, 1H, J=10.2 Hz), 4.69 (dd, 1H, J=9.9,5.4 Hz), 6.34 (s, 1H), 6.59 (d, 1H, J=7.5 Hz), 7.10 (d, 2H, J=9 Hz), 7.26-7.40 (m, 5H), 7.48 (d, 1H, J=7.5 Hz), 7.74 (d, 2H, J=9 Hz), 10.46 (s, 1H); ¹³C NMR (CDCl₃) δ 5.74, 20.81, 22.12, 28.14, 29.02, 47.87, 51.82, 51.85, 54.82, 57.77, 63.85, 107.42, 118.97, 125.55, 126.35, 127.55, 127.91, 128.18, 128.96, 139.74, 140.00, 152.88, 154.79, 155.60, 156.57, 159.87, 167.28; ES-MS m/z 599 (M+H). Anal. Calcd. for C₃₃H₃₈N₆O₅.0.2CH₂Cl₂.0.8CH₄O: C, 63.68; H, 6.54; N, 13.10. Found: C, 64.04; H, 6.30; N, 12.80.

### EXAMPLE 68

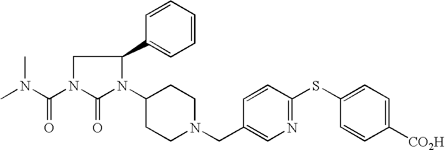

### COMPOUND 68: 4-{5-[4-((R)-3-Dimethylcarbamoyl-2-oxo-5-phenyl-imidazolidin-1-yl)-piperidin-1-ylmethyl]-pyridin-2-ylsulfanyl}-benzoic acid

COMPOUND 68 was isolated as a white foam (67.9 mg, 49%). ¹H NMR (CDCl₃) δ 1.50 (s, 2H), 1.80 (d, 1H, J=11.1 Hz), 2.21 (m, 2H), 2.36 (t, 1H, J=11.3 Hz), 3.03 (s, 7H), 3.31 (d, 1H, J=10.5 Hz), 3.42 (dd, 1H, J=9.8, 4.4 Hz), 3.62 (d, 1H, J=12.9 Hz), 3.68 (d, 1H, J=12.9 Hz), 3.88 (s, 1H), 4.10 (t, 1H, J=9.5 Hz), 4.64 (dd, 1H, J=8.9, 4.5 Hz), 6.67 (d, 1H, J=8.1 Hz), 7.17-7.26 (m, 5H), 7.46 (d, 1H, J=8.1 Hz), 7.60 (d, 2H, J=7.8 Hz), 7.98 (d, 2H, J=7.8 Hz), 8.25 (s, 1H); ¹³C NMR (CDCl₃) δ 27.69, 29.03, 38.32, 51.03, 51.54, 52.18, 52.25, 55.45, 58.19, 121.71, 126.19, 126.85, 128.99, 129.42, 131.29, 134.37, 134.85, 135.19, 139.31, 141.43, 151.10, 155.89, 156.39, 161.94, 169.97; ES-MS m/z 560 (M+H); Anal. Calcd. for C₃₀H₃₃N₅O₄S.1.4CH₂Cl₂: C, 55.50; H, 5.46; N, 10.31. Found: C, 55.34; H, 5.15; N, 10.33.

### EXAMPLE 69

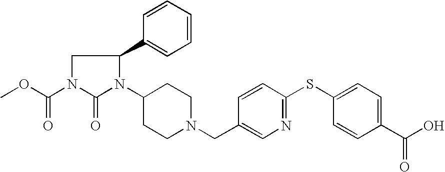

### COMPOUND 69: (R)-3-{1-[6-(4-Carboxy-phenylsulfanyl)-pyridin-3-ylmethyl]-piperidin-4-yl}-2-oxo-4-phenyl-imidazolidine-1-carboxylic acid methyl ester

COMPOUND 69 was isolated as a white powder (24.0 mg, 38%). ¹H NMR (CDCl₃) δ 1.48-1.58 (m, 2H), 1.81 (d, 1H, J=11.7 Hz), 2.19-2.45 (m, 3H), 3.08 (d, 1H, J=10.5 Hz), 3.35 (d, 1H, J=9.9 Hz), 3.70-3.89 (m, 3H), 3.85 (s, 3H), 3.92 (m, 1H), 4.12 (t, 1H, J=10.2 Hz), 4.63 (dd, 1H, J=9.6, 4.8 Hz), 6.64 (d, 1H, J=8.4 Hz), 7.15-7.23 (m, 5H), 7.46 (d, 1H, J=8.4 Hz), 7.62 (d, 2H, J=8.1 Hz), 7.97 (d, 1H, J=8.1 Hz), 8.26 (s, 1H); ¹³C NMR (CDCl₃) δ 27.20, 28.61, 30.08, 50.77, 51.27, 51.99, 52.14, 53.92, 54.91, 58.02, 121.50, 125.78, 126.84, 129.30, 129.62, 131.32, 134.57, 135.05, 139.41, 141.24, 151.20, 152.77, 154.00, 162.32, 169.97; ES-MS m/z 547 (M+H). Anal. Calcd. for C₂₉H₃₀N₄O₅S.0.9CH₂Cl₂: C, 57.64; H, 5.14; N, 8.99. Found: C, 57.50; H, 5.12; N, 8.95.

### EXAMPLE 70

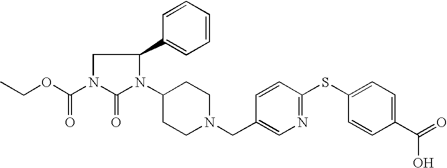

### COMPOUND 70: (R)-3-{1-[6-(4-Carboxy-phenylsulfanyl)-pyridin-3-ylmethyl]-piperidin-4-yl}-2-oxo-4-phenyl-imidazolidine-1-carboxylic acid ethyl ester

COMPOUND 70 was isolated as a white foam (127.3 mg, 96%). ¹H NMR (CDCl₃) δ 1.31 (t, 3H, J=6.0 Hz), 1.44-1.53 (m, 2H), 1.81 (d, 1H, J=10.5 Hz), 2.22-2.46 (m, 3H), 3.09 (d, 1H, J=9.9 Hz), 3.35 (d, 1H, J=9.9 Hz), 3.59-3.76 (m, 3H), 3.95 (br s, 1H), 4.11 (t, 1H, J=9.6 Hz), 4.27 (q, 2H, J=6.0 Hz), 4.62 (m, 1H), 6.65 (d, 1H, J=7.8 Hz), 7.07-7.26 (m, 5H), 7.46 (d, 1H, J=7.8 Hz), 7.60 (d, 2H, J=6.9 Hz), 7.96 (d, 2H, J=6.9 Hz), 8.27 (s, 1H); ¹³C NMR (CDCl₃) δ 14.73, 27.18, 28.67, 50.75, 51.01, 51.93, 54.78, 57.80, 62.99, 121.61, 125.58, 126.85, 129.25, 129.58, 131.31, 133.05, 134.91, 139.51, 141.30, 151.22, 152.09, 154.19, 162.26, 170.18; ES-MS m/z 561 (M+H); Anal. Calcd. for C₃₀H₃₂N₄O₅S.1.5CH₂Cl₂: C, 54.99; H, 5.13; N, 8.14. Found: C, 55.01; H, 4.78; N, 7.87.

### EXAMPLE 71

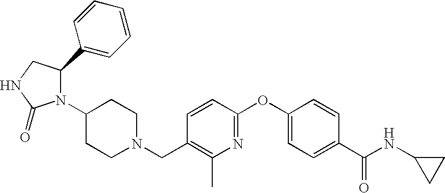

### COMPOUND 71: N-Cyclopropyl-4-{6-methyl-5-[4-((R)-2-oxo-5-phenyl-imidazolidin-1-yl)-piperidin-1-ylmethyl]-pyridin-2-yloxy}-benzamide

COMPOUND 71 was isolated as a yellow powder (28.9 mg, 35%). ¹H NMR (CDCl₃) δ 0.58-0.63 (m, 2H), 0.82-0.88 (m, 2H), 1.12-1.25 (m, 1H), 1.41 (d, 1H, J=11.7 Hz), 1.65 (d, 1H, J=10.5 Hz), 1.85-2.10 (m, 3H), 2.35 (s, 3H), 2.64 (d, 1H, J=10.8 Hz), 2.75-2.95 (m, 2H), 3.20 (t, 1H, J=7.5 Hz), 3.30 (s, 2H), 3.60 (m, 1H), 3.74 (t, 1H, J=9.0 Hz), 4.71 (dd, 1H, J=9.0, 6.6 Hz), 4.82 (s, 1H), 6.35 (br s, 1H), 6.58 (d, 1H, J=8.1 Hz), 7.09 (d, 2H, J=8.7 Hz), 7.28-7.40 (m, 5H), 7.48 (d, 1H, J=8.1 Hz), 7.73 (d, 2H, J=8.7 Hz); ¹³C NMR (CDCl₃) δ 7.1, 22.2, 23.5, 29.6, 31.2, 48.6, 52.2, 53.5, 53.6, 59.0, 59.2, 108.8, 120.3, 127.2, 128.0, 128.7, 129.0, 129.2, 130.3, 141.4, 142.8, 157.0, 158.1, 161.2, 162.9, 168.7; ES-MS m/z 526 (M+H). Anal. Calcd. for C₃₁H₃₅N₅O₃.0.5CH₂Cl₂.0.3C₆H₁₄: C, 67.34; H, 6.82; N, 11.79. Found: C, 67.21; H, 6.60; N, 11.75.

### EXAMPLE 72

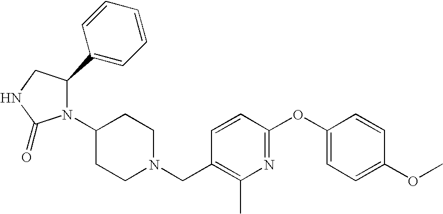

### COMPOUND 72: (R)-1-{1-[6-(4-Methoxy-phenoxy)-2-methyl-pyridin-3-ylmethyl]-piperidin-4-yl}-5-phenyl-imidazolidin-2-one

COMPOUND 72 was isolated as a white powder (460.9 mg, 67%). ¹H NMR (CDCl₃) δ 1.08-1.22 (m, 1H), 1.41 (d, 1H, J=12.3 Hz), 1.66 (d, 1H, J=11.4 Hz), 1.80-2.02 (m, 3H), 2.38 (s, 3H), 2.63 (d, 1H, J=12.3 Hz), 2.82 (d, 1H, J=9.9 Hz), 3.21 (t, 1H, J=8.1 Hz), 3.29 (s, 2H), 3.65 (tt, 1H, J=12.0, 3.6 Hz), 3.75 (t, 1H, J=9.0 Hz), 3.80 (s, 3H), 4.72 (dd, 1H, J=9.3, 6.3 Hz), 4.80 (s, 1H), 6.41 (d, 1H, J=8.1 Hz), 6.88 (dt, 2H, J=9.3, 2.4 Hz), 7.04 (dt, 2H, J=9.3 2.4 Hz), 7.27-7.41 (m, 6H); ¹³C NMR (CDCl₃) δ 20.9, 28.1, 29.8, 47.2, 50.8, 52.0, 52.2, 54.6, 57.5, 57.9, 105.4, 113.7, 121.0, 125.1, 125.8, 127.2, 127.8, 139.8, 141.5, 146.9, 155.3, 155.5, 161.5, 161.7; ES-MS m/z 473 (M+H). Anal. Calcd. for C₂₉H₃₂N₄O₃.0.4CH₂Cl₂: C, 67.34; H, 6.53; N, 11.06. Found: C, 67.43; H, 6.75; N, 10.91.

Examples 73 to 101 were prepared following the scheme illustrated below. RCHO is as defined in the table and X is as defined in the individual examples.

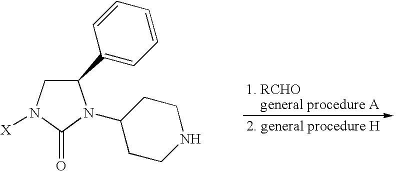

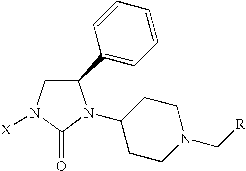

| Example | RCHO |
|---|---|
| 73 | 4-(4-formyl-3-methyl-phenoxy)-benzoic acid methyl ester |
| 74 | 4-(5-formyl-6-methyl-pyridin-2-yloxy)-benzoic acid methyl |
| | ester |
| 75 | 4-(5-formyl-pyrimidin-2-ylsulfanyl)-benzoic acid methyl ester |
| 76 | 4-(5-formyl-6-methyl-pyridin-2-yloxy)-benzoic acid methyl |
| | ester |
| 77 | 4-(5-formyl-pyrimidin-2-yloxy)-benzoic acid methyl ester |
| 78 | 4-(5-formyl-pyrimidin-2-ylsulfanyl)-benzoic acid methyl ester |
| 79 | 4-(5-formyl-6-methyl-pyridin-2-yloxy)-benzoic acid methyl |
| | ester |
| 80 | 4-(5-formyl-pyrimidin-2-ylsulfanyl)-benzoic acid methyl ester |
| 81 | 4-(5-formyl-6-methyl-pyridin-2-yloxy)-benzoic acid methyl |
| | ester |
| 82 | 4-(6-fluoro-5-formyl-pyridin-2-yloxy)-benzoic acid methyl |
| | ester |
| 83 | 4-(5-formyl-6-methyl-pyridin-2-yloxy)-benzoic acid methyl |
| | ester |
| 84 | 4-(5-formyl-6-methyl-pyridin-2-yloxy)-benzoic acid methyl |
| | ester |
| 85 | 4-(5-formyl-6-methyl-pyridin-2-yloxy)-benzoic acid methyl |
| | ester |
| 86 | 4-(5-formyl-6-methyl-pyridin-2-ylsulfanyl)-benzoic acid |
| | methyl ester |
| 87 | 4-(5-formyl-6-methyl-pyridin-2-yloxy)-benzoic acid methyl |
| | ester |
| 88 | [4-(5-formyl-6-methyl-pyridin-2-yloxy)-phenyl]-acetic acid |
| | methyl ester |
| 89 | 4-(5-formyl-pyridin-2-ylsulfanyl)-benzoic acid methyl ester |
| 90 | 4-(5-formyl-pyridin-2-ylsulfanyl)-benzoic acid methyl ester |
| 91 | 4-(5-formyl-6-methyl-pyridin-2-ylsulfanyl)-benzoic acid |
| | methyl ester |
| 92 | 4-(5-formyl-4,6-dimethyl-pyrimidin-2-yloxy)-benzoic acid |
| | methyl ester |
| 93 | 4-(5-formyl-4-methyl-pyrimidin-2-yloxy)-benzoic acid methyl |
| | ester |
| 94 | 4-(5-formyl-6-methyl-pyridin-2-ylsulfanyl)-benzoic acid |
| | methyl ester |
| 95 | 4-(5-formyl-6-methyl-pyridin-2-ylsulfanyl)-2-methyl-benzoic |
| | acid methyl ester |
| 96 | 4-(5-formyl-6-methyl-pyridin-2-ylsulfanyl)-benzoic acid |
| 97 | 4-(5-formyl-6-methyl-pyridin-2-ylsulfanyl)-benzoic acid |
| 98 | 4-(5-formyl-6-methyl-pyridin-2-yloxy)-benzoic acid methyl |
| | ester |
| 99 | 4-(5-formyl-6-methyl-pyridin-2-yloxy)-benzoic acid methyl |
| | ester |
| 100 | 4-(5-formyl-6-methyl-pyridin-2-ylsulfanyl)-benzoic acid |
| | methyl ester |
| 101 | 4-(5-formyl-6-methyl-pyridin-2-ylsulfanyl)-benzoic acid |
| | methyl ester |

### EXAMPLE 73

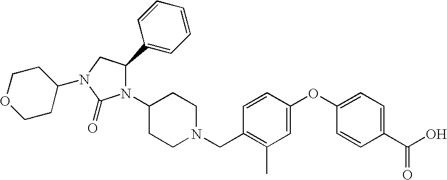

### COMPOUND 73: 4-(3-Methyl-4-{4-[(R)-2-oxo-5-phenyl-3-(tetrahydro-pyran-4-yl)-imidazolidin-1-yl]-piperidin-1-ylmethyl}-phenoxy)-benzoic acid

COMPOUND 73 was isolated as a white solid (53 mg, 45% over 2 steps). ¹H NMR (CD₃OD) δ 1.60-1.85 (m, 7H), 2.34 (dq, 1H, J=12.0, 3.6 Hz), 2.37 (s, 3H), 2.92 (dq, 2H, J=11.4, 3.3 Hz), 3.16 (m, 1H), 3.30-3.60 (m, 5H), 3.79 (t, 1H, J=9.0 Hz), 3.98 (m, 3H), 4.11 (s, 2H), 4.74 (m, 1H), 6.93 (dd, 1H, J=8.4, 2.6 Hz), 6.98 (d, 1H, J=2.4 Hz), 7.01 (d, 2H, J=6.9 Hz), 7.40 (m, 6H), 8.00 (d, 2H, J=6.9 Hz); ¹³C NMR (CD₃OD) δ 22.82, 30.71, 31.37, 33.71, 34.10, 52.44, 53.40, 54.16, 56.02, 56.10, 60.94, 61.10, 71.08, 71.18, 121.35, 121.93 (2C), 125.75, 128.56, 131.03 (2C), 132.59, 133.14 (2C), 135.93 (2C), 137.81, 145.47, 145.92, 161.50, 164.56, 164.92, 172.73; ES-MS m/z 570 (M+H). Anal. Calcd. for C₃₄H₃₉N₃O₅.0.9CH₂Cl₂: C, 64.88; H, 6.36; N, 6.50. Found: C, 64.90; H, 6.70; N, 6.51.

### EXAMPLE 74

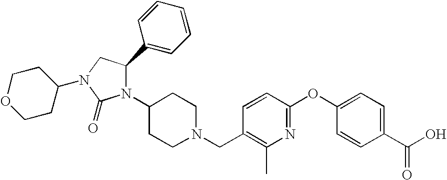

### COMPOUND 74: 4-(6-Methyl-5-{4-[(R)-2-oxo-5-phenyl-3-(tetrahydro-pyran-4-yl)-imidazolidin-1-yl]-piperidin-1-ylmethyl}-pyridin-2-yloxy)-benzoic acid

COMPOUND 74 was isolated as a white solid (164 mg, 51% over 2 steps). ¹H NMR (CD₃OD) δ 1.32 (dq, 1H, J=12.0, 3.6 Hz), 1.48 (br d, 1H, J=12.0 Hz), 1.60-1.80 (m, 5H), 1.95-2.20 (m, 3H), 2.39 (s, 3H), 2.78 (d, 1H, J=11.4 Hz), 2.94 (d, 1H, J=11.1 Hz), 3.13 (m, 1H), 3.40-3.60 (m, 5H), 3.77 (t, 1H, J=9.0 Hz), 3.93 (m, 3H), 4.73 (m, 1H), 6.69 (d, 1H, J=8.1 Hz), 7.07 (d, 2H, J=8.7 Hz), 7.30-7.40 (m, 5H), 7.64 (d, 1H, J=8.4 Hz), 8.01 (d, 2H, J=8.7 Hz); ¹³C NMR (CD₃OD) δ 20.71, 28.44, 29.82 (2C), 30.14, 48.57, 49.44, 52.08, 52.85, 53.05, 56.74, 57.98, 67.18, 67.27, 109.02, 119.53 (2C), 125.88, 127.02 (2C), 128.46, 129.03 (2C), 130.67, 131.50 (2C), 142.62, 142.91, 157.37, 158.09, 160.87, 162.01, 171.02; ES-MS m/z 571 (M+H). Anal. Calcd. for C₃₃H₃₈N₄O₅.0.7CH₂Cl₂.0.3NH₃: C, 63.38; H, 6.45; N, 9.87. Found: C, 63.20; H, 6.60; N, 9.85.

### EXAMPLE 75

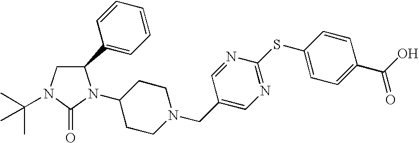

### COMPOUND 75: 4-{5-[4-((R)-3-tert-Butyl-2-oxo-5-phenyl-imidazolidin-1-yl)-piperidin-1-ylmethyl]-pyrimidin-2-ylsulfanyl}-benzoic acid

COMPOUND 75 was isolated as a white solid (21 mg, 35% over 2 steps). ¹H NMR (CD₃OD) δ 1.35-1.52 (m, 10H), 1.59-1.63 (m, 1H), 1.75-1.79 (m, 1H), 2.02-2.14 (m, 1H), 2.41-2.54 (m, 2H), 3.02-3.06 (m, 1H), 3.12-3.17 (m, 2H), 3.53-3.61 (m, 1H), 3.75-3.84 (m, 3H), 4.60 (dd, 1H, J=8.7, 7.2 Hz), 7.28-7.39 (m, 5H), 7.66 (br s, 2H), 8.04 (br s, 2H), 8.49 (s, 2H); ES-MS m/z 546 (M+1).

### EXAMPLE 76

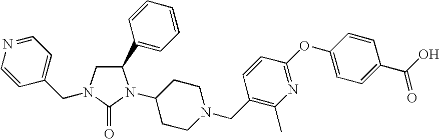

### COMPOUND 76: 4-{6-Methyl-5-[4-((R)-2-oxo-5-phenyl-3-pyridin-4-ylmethyl-imidazolidin-1-yl)-piperidin-1-ylmethyl]-pyridin-2-yloxy}-benzoic acid

A solution of ((R)-2-amino-1-phenyl-ethyl)-carbamic acid tert-butyl ester (489, 2.07 mmol) and 4-pyridinecarboxaldehyde (222 mg, 2.07 mmol) in MeOH (10 mL) was stirred at room temperature for 1.3 hours. NaBH₄ (78 mg, 2.1 mmol) was added portionwise and the mixture stirred for 10 minutes at room temperature. Aqueous work-up afforded the carbamate. Following general procedure C, the carbamate afforded (R)-1-phenyl-N²-pyridin-4-ylmethyl-ethane-1,2-diamine as a colourless oil (283 mg, 60% over 3 steps).

Following general procedure A, the above amine (281 mg, 1.24 mmol) and 1-BOC-4-piperidone (259 mg, 1.30 mmol) followed by standard work-up and purification afforded the desired substrate (224 mg, 44%). Following general procedure K: to a solution of the above diamine (224 mg, 0.546 mmol) and Et₃N (0.15 mL, 1.1 mmol) in CH₂Cl₂ (10 mL) at 0° C. was added a solution of triphosgene (81 mg, 0.27 mmol) in CH₂Cl₂ (0.5 mL). The mixture was stirred at room temperature for 2 hours. Standard work-up and purification afforded 4-((R)-2-oxo-5-phenyl-3-pyridin-4-ylmethyl-imidazolidin-1-yl)-piperidine-1-carboxylic acid tert-butyl ester (105 mg, 44%). Following general procedure C the above substrate afforded (R)-4-phenyl-3-piperidin-4-yl-1-pyridin-4-ylmethyl-imidazolidin-2-one as a yellow foam (74 mg, 91%).

COMPOUND 76 was isolated as a yellow foam (22 mg, 34% over 2 steps). ¹H NMR (CD₃OD) δ 1.88-2.03 (m, 3H), 2.38-2.46 (m, 4H), 3.06-3.16 (m, 3H), 3.37-3.54 (m, 2H), 3.61-3.70 (m, 1H), 3.77 (t, 1H, J=9.0 Hz), 4.29 (s, 2H), 4.50 (dd, 2H, J=24.6, 16.5 Hz), 4.81-4.87 (m, 1H), 6.91 (d, 1H, J=8.1 Hz), 7.19 (d, 2H, J=8.7 Hz), 7.33-7.46 (m, 7H), 7.85 (d, 1H, J=8.4 Hz), 8.07 (d, 2H, J=8.4 Hz), 8.54 (br s, 2H); ES-MS m/z 578 (M+1). Anal. Calcd. for C₃₄H₃₅N₅O₄.1.2CH₂Cl₂.1.8CH₄O: C, 60.28; H, 6.10; N, 9.50. Found: C, 60.48; H, 6.02; N, 9.35.

### EXAMPLE 77

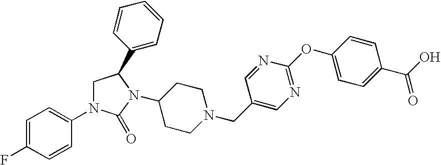

### COMPOUND 77: 4-(5-{4-[(R)-3-(4-Fluoro-phenyl)-2-oxo-5-phenyl-imidazolidin-1-yl]-piperidin-1-ylmethyl}-pyrimidin-2-yloxy)-benzoic acid

Following general procedure E: to a solution of tert-butoxycarbonylamino-phenyl-acetic acid (450 mg, 1.80 mmol) and 4-fluoroaniline (0.188 mL, 1.96 mmol) in DMF (18 mL) was added EDCI (374 mg, 1.96 mmol), HOBt (265 mg, 1.96 mmol) and NMM (519 μL, 3.6 mmol). The reaction mixture was allowed to stir over 3 days at room temperature to afford [(R)-(4-fluoro-phenylcarbamoyl)-phenyl-methyl]-carbamic acid tert-butyl ester (575 mg, 93%) after work-up and purification.

Following general procedure C, the Boc-protected amine above (575 mg, 1.7 mmol) afforded (R)-2-amino-N-(4-fluoro-phenyl)-2-phenyl-acetamide (296 mg, 71%).

To a solution of the amide above (296 mg, 1.21 mmol) in THF (6 mL) was added BH₃.THF (1.0 M in THF, 3.6 mL, 3.6 mmol). The reaction mixture was allowed to stir overnight at 60° C., cooled to room temperature, and then quenched with MeOH (4 mL). The solvents were evacuated in vacuo and 6N HCl (5 mL) was added and the reaction mixture was heated to 80° C. for 2 hours. Basic work-up afforded the diamine as a brown oil (242 mg, 87%).

Following general procedure A: to a solution of the above amine (242 mg, 1.05 mmol) in dichloromethane (10.5 mL) was added N-boc-piperidone (239 mg, 1.2 mmol) followed by NaBH(OAc)₃ (318 mg, 1.5 mmol) and the reaction mixture was allowed to stir overnight at room temperature. Standard work-up and purification afforded 4-[(R)-2-(4-fluoro-phenylamino)-1-phenyl-ethylamino]-piperidine-1-carboxylic acid tert-butyl ester (346 mg, 88%).

Following general procedure K: to a solution of the above diamine (346 mg, 0.84 mmol) in dichloromethane (8.4 mL) at 0° C. under argon was added pyridine (135 μL, 1.67 mmol) followed by triphosgene (124 mg, 0.42 mmol) and the mixture was stirred at 0° C. for 1 hour. Standard work-up and purification afforded 4-[(R)-3-(4-fluoro-phenyl)-2-oxo-5-phenyl-imidazolidin-1-yl]-piperidine-1-carboxylic acid tert-butyl ester (327 mg, 88%).

Following general procedure C, the above substrate (327 mg, 0.74 mmol) afforded (R)-1-(4-fluoro-phenyl)-4-phenyl-3-piperidin-4-yl-imidazolidin-2-one (271 mg, quant).

COMPOUND 77 was isolated as a white solid (38 mg, 45% over 2 steps). ¹H NMR (CDCl₃) δ 1.31-1.47 (m, 1H), 1.55 (br d, 1H, J=11.4 Hz), 1.85 (br d, 1H, J=11.1 Hz), 2.19-2.26 (m, 2H), 2.33-2.41 (m, .1H), 3.00 (br d, 1H, J=10.5 Hz), 3.26 (br d, 1H, J=9.9 Hz), 3.46 (s, 2H), 3.55-3.68 (m, 1H), 3.90-3.99 (m, 1H), 4.13 (t, 1H, J=9.3 Hz), 4.75 (dd, 1H, J=9.0, 6.0 Hz), 6.35 (br s, 1H), 6.98-7.04 (m, 2H), 7.18-7.22 (m, 5H), 7.31-7.34 (m, 2H), 7.44-7.49 (m, 2H), 8.02 (d, 2H, J=8.4 Hz), 8.48 (s, 2H); ¹³C NMR (CDCl₃) δ 27.9, 29.7, 51.1, 51.4, 52.8, 53.1, 55.4, 56.1, 115.7, 116.0, 119.6, 119.7, 121.8, 127.1, 129.1, 129.5, 132.1, 136.5, 142.0, 156.8, 157.9, 161.6, 165.3, 169.3; ES-MS m/z 568 (M+H). Anal. Calcd. for C₃₂H₃₀N₅FO₄.0.5CH₂Cl₂.1.0CH₃OH: C, 62.66; H, 5.49; N, 10.91. Found: C, 62.63; H, 5.49; N, 10.94.

### EXAMPLE 78

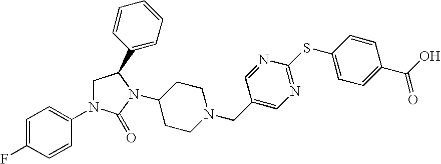

### COMPOUND 78: 4-(5-{4-[(R)-3-(4-Fluoro-phenyl)-2-oxo-5-phenyl-imidazolidin-1-yl]-piperidin-1-ylmethyl}-pyrimidin-2-ylsulfanyl)-benzoic acid

See EXAMPLE 77 for preparation of 1-(4-fluoro-phenyl)-4-phenyl-3-piperidin-4-yl-imidazolidin-2-one. COMPOUND 78 was isolated as a white solid (50 mg, 55% over 2 steps). ¹H NMR (DMSO) δ 1.08-1.17 (m, 1H), 1.30-1.35 (m, 1H), 1.50-1.56 (m, 1H), 1.75-2.04 (m, 3H), 2.36-2.80 (m, 2H), 3.13-3.50 (m, 4H), 4.09-4.20 (m, 1H), 4.84-4.89 (m, 1H), 7.10-7.16 (m, 2H), 7.32-7.41 (m, 5H), 7.54-7.62 (m, 2H), 7.70 (d, 2H, J=8.1 Hz), 7.96 (d, 2H, J=8.4 Hz), 8.45 (s, 2H); ES-MS m/z 584 (M+H). Anal. Calcd. for C₃₂H₃₀N₅O₃F.0.9CH₂Cl₂.0.7CH₃OH: C, 59.13; H, 5.11; N, 10.26. Found: C, 59.03; H, 5.12; N, 10.30.

### EXAMPLE 79

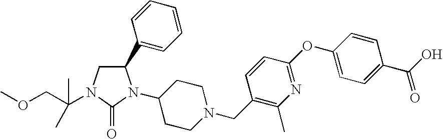

### COMPOUND 79: 4-(5-{4-[(R)-3-(2-Methoxy-1,1-dimethyl-ethyl)-2-oxo-5-phenyl-imidazolidin-1-yl]-piperidin-1-ylmethyl}-6-methyl-pyridin-2-yloxy)-benzoic acid

Following general procedure F: to a solution of ((R)-2-amino-1-phenyl-ethyl)-carbamic acid tert-butyl ester (732 mg, 2.91 mmol) in THF (20 mL) at 0° C. was added NMM (0.36 mL, 3.5 mmol) followed by IBCF (0.38 mL, 2.9 mmol) and the mixture stirred for 5 minutes. A solution of 2-amino-2-methyl-1-propanol (310 mg, 3.48 mmol) in THF (10 mL) was added and the mixture stirred at 0° C. for 30 min and at room temperature overnight. Standard work-up and purification gave [(R)-2-(2-hydroxy-1,1-dimethyl-ethylamino)-1-phenyl-ethyl]-carbamic acid tert-butyl ester (632 mg, 98%).

To a solution of the above alcohol (632 mg, 2.0 mmol) in THF (30 mL) at 0° C. was added NaH (60%, 98 mg, 2.5 mmol) slowly. The mixture was stirred for 15 minutes then MeI (0.16 mL, 2.5 mmol) was added at 0° C. Standard work-up and purification afforded [(R)-2-(2-methoxy-1,1-dimethyl-ethylamino)-1-phenyl-ethyl]-carbamic acid tert-butyl ester (200 mg, 31%).

Following general procedure C with the above carbamate (200 mg) gave the crude intermediate. Reduction with BH₃.THF (1.0M in THF, 3.0 mL, 3.0 mmol) in THF (4 mL) at reflux followed by treatment with 6N HCl (2 mL) and subsequent basic work-up and purification afforded the amine (165 mg). Following general procedure A, the above amine and 1-BOC-4-piperidone (150 mg, 0.75 mmol) afforded 4-[(R)-2-(2-methoxy-1,1-dimethyl-ethylamino)-1-phenyl-ethylamino]-piperidine-1-carboxylic acid tert-butyl ester (230 mg, 91% over 3 steps).

Following general procedure K: to a solution of the diamine (256 mg, 0.631 mmol) and pyridine (0.110 mL) in CH₂Cl₂ (6 mL) at 0° C. was added triphosgene (97 mg, 0.32 mmol) and the mixture was stirred a room temperature for 2 hours. Standard work-up and purification afforded 4-[(R)-3-(2-methoxy-1,1-dimethyl-ethyl)-2-oxo-5-phenyl-imidazolidin-1-yl]-piperidine-1-carboxylic acid tert-butyl ester. Following general procedure C, the above substrate gave (R)-1-(2-methoxy-1,1-dimethyl-ethyl)-4-phenyl-3-piperidin-4-yl-imidazolidin-2-one (184 mg, 87% over 2 steps).

COMPOUND 79 was isolated as a white solid (89 mg, 92% over 2 steps). ¹H NMR (CDCl₃) δ 1.34 (d, 6H, J=7.2 Hz), 1.35-1.42 (m, 1H), 1.76-1.89 (m, 2H), 2.39 (s, 3H), 2.54-2.66 (m, 1H), 2.81-8.86 (m, 1H), 3.20-3.34 (m, 5H), 3.48-3.56 (m, 3H), 3.81 (t, 1H, J=9.0 Hz), 4.00-4.14 (m, 3H), 4.51-4.59 (m, 1H), 6.76 (d, 1H, J=9.0 Hz), 7.12 (d, 2H, J=9.0 Hz), 7.21-7.38 (m, 5H), 8.02 (d, 2H, J=9.0 Hz), 8.26 (br s, 1H); ¹³C NMR (CDCl₃) δ 22.40, 23.45, 49.17, 51.65, 53.45, 56.09, 59.17, 78.33, 109.90, 120.60, 126.63, 126.96, 128.45, 129.05, 131.74, 144.27, 157.02, 157.84, 160.47, 162.45, 168.76; ES-MS m/z 573 (M+1). Anal. Calcd. for C₃₁H₃₇N₅O₄S.0.13H₂O.1.22CH₂Cl₂: C, 60.54; H, 6.34; N, 8.25. Found: C, 60.53; H, 6.32; N, 8.33.

### EXAMPLE 80

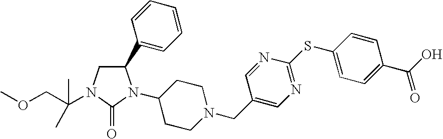

### COMPOUND 80: 4-(5-{4-[(R)-3-(2-Methoxy-1,1-dimethyl-ethyl)-2-oxo-5

-phenyl-imidazolidin-1-yl]-piperidin-1-ylmethyl}-pyrimidin-2-ylsulfanyl)-benzoic acid

See EXAMPLE 79 for preparation of (R)-1-(2-methoxy-1,1-dimethyl-ethyl)-4-phenyl-3-piperidin-4-yl-imidazolidin-2-one. COMPOUND 80 was isolated as a white solid (67 mg, 73% over 2 steps). ¹H NMR (CDCl₃) δ 1.18-1.35 (m, 8H), 1.78-1.85 (m, 1H), 2.30-2.73 (m, 3H), 3.11-3.15 (m, 1H), 3.25-3.30 (m, 2H), 3.34 (s, 3H), 3.46-3.56 (m, 3H), 3.74-3.93 (m, 3H), 4.48-4.54 (m, 1H), 7.17-7.24 (m, 3H), 7.61 (d, 2H, J=8.1 Hz), 7.98 (d, 2H, J=8.1 Hz), 8.60 (br s, 2H); ¹³C NMR (CDCl₃) δ 23.90, 26.36, 28.81, 49.92, 51.93, 55.69, 56.43, 59.57, 78.76, 127.37, 128.81, 129.34, 130.93, 132.42, 134.47, 135.15, 142.37, 160.14, 160.84, 168.91, 173.47; ES-MS m/z 576 (M+1). Anal. Calcd. for C₃₁H₃₇N₅O₄S.0.79CH₄O.0.56CH₂Cl₂: C, 59.90; H, 6.41; N, 10.80. Found: C, 59.89; H, 6.43; N, 10.82.

### EXAMPLE 81

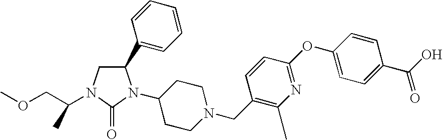

### COMPOUND 81: 4-(5-{4-[(R)-3-((S)-2-Methoxy-1-methyl-ethyl)-2-oxo-5-phenyl-imidazolidin-1-yl]-piperidin-1-ylmethyl}-6-methyl-pyridin-2-yloxy)-benzoic acid

A solution of (S)-2-amino-1-propanol (1.03 g, 13.7 mmol), phthalic anhydride (2.03 g, 13.7 mmol) and Et₃N (2.10 mL, 15.1 mmol) in toluene (17 mL) was heated to reflux for 21 hours. The mixture was concentrated under reduced pressure and filtered through a silica gel plug (7:3, CH₂Cl₂/EtOAc) to afford 2-((S)-2-hydroxy-1-methyl-ethyl)-isoindole-1,3-dione as colourless crystals (2.55 g, 91%).

A solution of the above alcohol (2.55 g, 12.4 mmol), MeI (2.33 mL, 37.3 mmol) and Ag₂O (8.64 g, 37.3 mmol) in CH₃CN (25 mL) was heated to reflux for 12 hours in the dark. Filtration and purification afforded 2-((S)-2-methoxy-1-methyl-ethyl)-isoindole-1,3-dione as a yellow oil (1.91 g, 70%).

The above phthalimide (1.91 g, 8.71 mmol) and hydrazine hydrate (0.30 mL, 9.6 mmol) in EtOH (2.0 mL) was heated to 75° C. for 4.5 hours in a sealed tube. The mixture was cooled to room temperature and crude (S)-2-methoxy-1-methyl-ethylamine was used in the next reaction without purification. Following general procedure F: to a solution of (R)-tert-butoxycarbonylamino-phenyl-acetic acid (575 mg, 2.29 mmol) in THF (11 mL) at 0° C. was added NMM (231 mg, 2.28 mmol) in THF (0.5 mL) followed by IBCF (0.30 mL, 2.3 mmol). The above amine was added and the mixture was stirred at room temperature for 15 hours. Standard work-up and purification afforded [(R)-((S)-2-methoxy-1-methyl-ethylcarbamoyl)-phenyl-methyl]-carbamic acid tert-butyl ester as a yellow solid (725 mg, 98%).

Following general procedure C, the above carbamate (725 mg, 2.25 mmol) afforded the amine which was subsequently reduced with BH₃.THF (1.0M in THF, 8.3 mL, 8.3 mmol) in THF (20 mL) at reflux. The mixture was treated with MeOH followed by 6N HCl. Basic work-up afforded the diamine as a yellow oil (375 mg, 80% over 2 steps).

Following general procedure A, the above diamine (375 mg, 1.80 mmol) and 1-BOC-4-piperidone (377 mg, 1.87 mmol) afforded the desired substrate (572 mg, 81%). Following general procedure K: to a solution of above substrate (572 mg, 1.46 mmol) and Et₃N (0.41 mL, 2.9 mmol) in CH₂Cl₂ (20 mL) at 0° C. was added triphosgene (217 mg, 0.731 mmol) in CH₂Cl₂ (0.5 mL). Standard work-up and purification afforded 4-[(R)-3-((S)-2-methoxy-1-methyl-ethyl)-2-oxo-5-phenyl-imidazolidin-1-yl]-piperidine-1-carboxylic acid tert-butyl ester (613 mg, quant).

COMPOUND 81 was isolated as a colourless foam (67 mg, 70% over 2 steps). ¹H NMR (CD₃OD) δ 1.10 (d, 3H, J=6.9 Hz), 1.76-1.94 (m, 3H), 2.33-2.44 (m, 1H), 2.45 (s, 3H), 3.03-3.19 (m, 3H), 3.30-3.65 (m, 8H), 3.76 (t, 1H, J=9.3 Hz), 4.11-4.23 (m, 1H), 4.27 (s, 2H), 4.74 (dd, 1H, J=9.3, 7.5 Hz), 6.90 (d, 1H, J=8.4 Hz), 7.17-7.20 (m, 2H), 7.33-7.41 (m, 5H), 7.85 (d, 1H, J=8.4 Hz), 8.04-8.08 (m, 2H); ¹³C NMR (CDCl₃) δ 14.31, 22.45, 25.90, 27.93, 47.21, 48.64, 49.00, 51.74, 52.24, 54.99, 56.18, 58.86, 73.95, 110.04, 117.99, 120.73, 126.39, 126.70, 128.45, 129.18, 131.75, 142.56, 144.56, 157.06, 157.82, 160.33, 162.57, 168.57; ES-MS m/z 559 (M+1). Anal. Calcd. for C₃₂H₃₈N₄O₅.0.8CH₂Cl₂.0.9CH₄O: C, 61.75; H, 6.64; N, 8.55. Found: C, 61.98; H, 6.72; N, 8.73.

### EXAMPLE 82

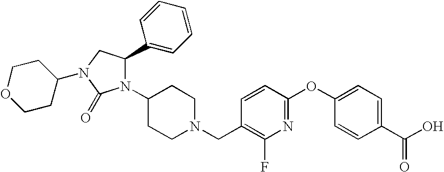

### COMPOUND 82: 4-(6-Fluoro-5-{4-[(R)-2-oxo-5-phenyl-3-(tetrahydro-pyran-4-yl)-imidazolidin-1-yl]-piperidine-1-ylmethyl}-pyridin-2-yloxy)-benzoic acid

COMPOUND 82 was isolated as a white solid (58 mg, 48% over 2 steps). ¹H NMR (CDCl₃) δ 1.27 (dq, 1H, J=12.0, 3.6 Hz), 1.48 (d, 1H, J=12.0 Hz), 1.66 (m, 4H), 1.75 (d, 1H, J=11.7 Hz), 2.18 (m, 2H), 2.35 (t, 1H, J=11.4 Hz), 2.97 (d, 1H, J=11.4 Hz), 3.09 (m, 1H), 3.25 (d, 1H, J=11.4 Hz), 3.49 (m, 3H), 3.64 (m, 2H), 3.86 (m, 1H), 4.03 (m, 3H), 4.58 (m, 1H), 6.62 (d, 1H, J=8.1 Hz), 7.13 (m, 4H), 7.24 (m, 1H), 7.69 (t, 1H, J=8.7 Hz), 7.97 (d, 2H, J=8.7 Hz); ES-MS m/z 575 (M+H).

### EXAMPLE 83

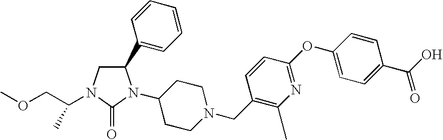

### COMPOUND 83: 4-(5-{4-[(R)-3-((R)-2-Methoxy-1-methyl-ethyl)-2-oxo-5-phenyl-imidazolidin-1-yl]-piperidin-1-ylmethyl}-6-methyl-pyridin-2-yloxy)-benzoic acid

4-[(R)-3-((R)-2-methoxy-1-methyl-ethyl)-2-oxo-5-phenyl-imidazolidin-1-yl]-piperidine-1-carboxylic acid tert-butyl ester was prepared using the same chemistry as 4-[(R)-3-((S)-2-methoxy-1-methyl-ethyl)-2-oxo-5-phenyl-imidazolidin-1-yl]-piperidine-1-carboxylic acid tert-butyl ester (see EXAMPLE 81) except that (R)-2-amino-1-propanol was used in lieu of (S)-2-amino-1-propanol. COMPOUND 83 was isolated as a colourless foam (80 mg, 80% over 2 steps). ¹H NMR (CD₃OD) δ 1.12 (d, 3H, J=6.9 Hz), 1.74-1.90 (m, 3H), 2.31-2.44 (m, 1H), 2.45 (s, 3H), 3.00-3.12 (m, 2H), 3.17 (dd, 1H, J=9.0, 7.2 Hz), 3.30-3.51 (m, 7H), 3.53-3.70 (m, 1H), 3.80 (t, 1H, J=9.3 Hz), 4.14-4.23 (m, 1H), 4.27 (s, 2H), 4.72 (dd, 1H, J=9.6, 7.2 Hz), 6.90 (d, 1H, J=8.4 Hz), 7.16-7.20 (m, 2H), 7.31-7.46 (m, 5H), 7.86 (d, 1H, J=8.7 Hz), 8.04-8.08 (m, 2H); ¹³C NMR (CDCl₃) δ 0.38, 14.62, 22.85, 26.13, 28.16, 47.44, 48.65, 49.35, 52.35, 55.48, 59.14, 74.18, 110.29, 118.62, 121.01, 126.94, 127.28, 128.81, 129.46, 132.09, 143.01, 144.82, 157.57, 158.13, 160.55, 162.90, 168.78; ES-MS m/z 559 (M+1). Anal. Calcd. for C₃₂H₃₈N₄O₅.0.8CH₂Cl₂.0.9H₂O: C, 61.28; H, 6.49; N, 8.72. Found: C, 61.44; H, 6.47; N, 8.83.

### EXAMPLE 84

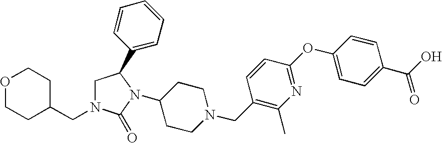

### COMPOUND 84: 4-(6-Methyl-5-{4-[(R)-2-oxo-5-phenyl-3-(tetrahydro-pyran-4-ylmethyl)-imidazolidin-1-yl]-piperidin-1-ylmethyl}-pyridin-2-yloxy)-benzoic acid

See EXAMPLE 52 for preparation of (R)-4-phenyl-3-piperidin-4-yl-1-(tetrahydro-pyran-4-ylmethyl)-imidazolidin-2-one. COMPOUND 84 was isolated as a white solid (47 mg, 57% over 2 steps). ¹H NMR (CDCl₃) δ 1.23-1.38 (m, 3H), 1.49-1.53 (m, 2H), 1.72-1.93 (m, 3H), 2.40 (s, 3H), 2.56-2.71 (m, 2H), 2.80-2.95 (m, 1H), 2.91-3.03 (m, 1H), 3.08-3.19 (m, 2H), 3.29-3.36 (m, 3H), 3.60 (br d, 1H, J=9.3 Hz), 3.72 (t, 1H, J=9.2 Hz), 3.95 (d, 2H, J=10.8 Hz), 4.12-4.22 (m, 3H), 4.72 (dd, 1H, J=9.0, 5.5 Hz), 6.78 (d, 1H, J=9.0 Hz), 7.11 (d, 2H, J=9 Hz), 7.22-7.37 (m, 5H), 8.00 (d, 2H, J=9.0 Hz), 8.34 (d, 1H, J=9.0 Hz); ¹³C NMR (CDCl₃) δ 22.3, 22.8, 23.7, 26.2, 28.2, 30.1, 31.0, 34.2, 34.4, 49.1, 49.5, 50.2, 51.4, 52.1, 52.5, 53.6, 54.0, 55.2, 56.6, 59.4, 67.9, 110.4, 118.3, 118.9, 121.1, 126.8, 127.0, 127.4, 128.9, 129.6, 129.8, 132.1, 139.9, 142.7, 144.1, 145.0, 157.5, 158.1, 161.3, 163.0, 168.8; ES-MS m/z 585 (M+H). Anal. Calcd. for C₃₄H₄₀N₄O_{5.}1.7CH₂Cl₂: C, 58.81; H, 6.00, N, 7.68. Found: C, 58.90; H, 6.27; N, 7.72.

### EXAMPLE 85

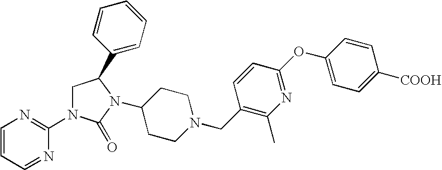

### COMPOUND 85: 4-{6-Methyl-5-[4-((R)-2-oxo-5-phenyl-3-pyrimidin-2-yl-imidazolidin-1-yl)-piperidin-1-ylmethyl]-pyridin-2-yloxy)-benzoic acid

Following general procedure G: to a solution of ((R)-2-amino-1-phenyl-ethyl)-carbamic acid tert-butyl ester (150 mg, 0.63 mmol) in DMF (1.26 mL) was added 2-bromopyrimidine (111 mg, 0.70 mmol) followed by DIPEA (132 μL, 0.76 mmol). The reaction mixture was allowed to stir at 85° C. overnight. Standard work-up and purification afforded [(R)-1-phenyl-2-(pyrimidin-2-ylamino)-ethyl]-carbamic acid tert-butyl ester (113 mg, 54%).

Following general procedure C, the above substrate (113 mg, 0.34 mmol) afford (R)-1-phenyl-N²-pyrimidin-2-yl-ethane-1,2-diamine (67 mg, 92%).

Following general procedure A: to a solution of the above amine (67 mg, 0.31 mmol) in DCM (3.1 mL) was added N-boc piperidone (68 mg, 0.34 mmol) followed by NaBH(OAc)₃ (91 mg, 0.43 mmol) and the mixture was stirred at rt for 19 hours. Standard work-up and purification afforded 4-[(R)-1-phenyl-2-pyrimidin-2-ylamino-ethylamino]-piperidine-1-carboxylic acid tert-butyl ester (96 mg, 78%).

Following general procedure K, to a solution of the above diamine (96 mg, 0.24 mmol) in DCM (2.4 mL) at 0° C. was added pyridine (39 μL, 0.48 mmol) followed by triphosgene (36 mg, 0.12 mmol) and the mixture was stirred at 0° C. for 1 hour. Standard work-up and purification afforded 4-((R)-2-oxo-5-phenyl-3-pyrimidin-2-yl-imidazolidin-1-yl)-piperidine-1-carboxylic acid tert-butyl ester (23 mg, 23%).

Following general procedure C, the above substrate (23 mg, 0.054 mmol) in DCM (0.22 mL) afforded (R)-4-phenyl-3-piperidin-4-yl-1-pyrimidin-2-yl-imidazolidin-2-one (20 mg, quant).

COMPOUND 85 was isolated as a white solid (11 mg, 32% over 2 steps). ¹H NMR (CDCl₃) δ 0.83-0.98 (m, 1H), 1.00-1.50 (m, 2H), 2.03-2.11 (m, 1H), 2.45-2.84 (m, 5H), 3.25-3.35 (m, 1H), 3.60-3.65 (m, 1H), 3.80-4.43 (m, 5H), 4.85-4.88 (m, 1H), 6.82 (d, 1H, J=8.4 Hz), 6.90-7.00 (m, 1H), 7.16 (d, 2H, J=8.4 Hz), 7.26-7.40 (m, 5H), 8.06 (d, 2H, J=8.4 Hz), 8.33-8.36 (m, 1H), 8.61 (d, 2H, J=4.5 Hz); ES-MS m/z 565 (M+H). Anal. Calcd. for C₃₂H₃₂N₆O₄.1.9CH₂Cl₂.0.4H₂O.1.1C₄H₈O: C, 56.62; H, 5.63; N,10.34. Found: C, 56.78; H, 5.64; N, 10.37.

### EXAMPLE 86

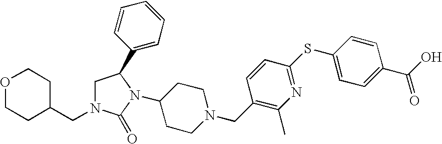

### COMPOUND 86: 4-(6-Methyl-5-{4-[(R)-2-oxo-5-phenyl-3-(tetrahydro-pyran-4-ylmethyl)-imidazolidin-1-yl]-piperidin-1-ylmethyl}-pyridin-2-ylsulfanyl)-benzoic acid

See EXAMPLE 52 for preparation of (R)-4-phenyl-3-piperidin-4-yl-1-(tetrahydro-pyran-4-ylmethyl)-imidazolidin-2-one. COMPOUND 86 was isolated as a white solid (39 mg, 51% over 2 steps). ¹H NMR (CDCl₃) δ 1.20-1.51 (m, 7H), 1.76-1.90 (m, 3H), 2.52 (s, 3H), 2.55-2.70 (m, 1H), 2.80-2.90 (m, 1H), 3.02-3.36 (m, 6H), 3.50-3.60 (m, 1H), 3.70 (t, 1H, J=9.3 Hz), 3.95 (d, 2H, J=10.5 Hz), 4.04-4.13 (m, 2H), 4.64-4.67 (m, 1H), 6.77 (d, 1H, J=9.0 Hz), 7.22-7.31 (m, 5H), 7.57 (d, 2H, J=6.0 Hz), 7.97-8.00 (m, 3H); ¹³C NMR (CDCl₃) δ 21.7, 24.7, 26.8, 29.6, 32.8, 48.3, 48.9, 50.8, 52.6, 54.1, 55.2, 66.5, 118.8, 125.6, 127.5, 128.2, 129.9, 130.4, 133.1, 135.1, 140.0, 141.0, 156.9, 159.9, 160.5, 167.3; ES-MS m/z 601 (M+H). Anal. Calcd. for C₃₄H₄₀N₄O₄S.0.5CH₂Cl₂.1.6H₂O: C, 61.66; H, 6.63; N, 8.34. Found: C, 61.60; H, 6.69; N, 8.04.

### EXAMPLE 87

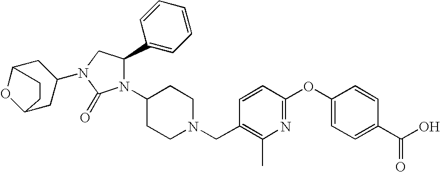

### COMPOUND 87: 4-(6-Methyl-5-{4-[(R)-3-(8-oxa-bicyclo[3.2.1]oct-3-yl)-2-oxo-5-phenyl-imidazolidin-1-yl]-piperidin-1-ylmethyl}-pyridin-2-yloxy)-benzoic acid

COMPOUND 87 was isolated as a white powder (48.0 mg, 54% over 2 steps). ¹H NMR (CD₃OD) δ 1.48-1.63 (m, 4H), 1.63-1.75 (m, 3H), 1.80-2.00 (m, 2H), 2.18 (br s, 3H), 2.42 (s, 3H), 2.58 (br s, 2H), 3.09 (br s, 2H), 3.22 (br s, 1H), 3.60 (br s, 1H), 3.71 (br s, 1H), 3.87 (s, 2H), 4.02 (br s, 1H), 4.40 (br s, 2H), 4.71 (br s, 1H), 6.79 (br s, 1H), 7.13 (br s, 2H), 7.36 (br s, 5H), 7.75 (br s, 1H), 8.03 (br s, 2H); ¹³C NMR (CD₃OD) δ 21.1, 27.5, 28.5, 29.8, 31.5, 31.6, 33.4, 33.7, 43.6, 49.7, 50.9, 52.4, 52.5, 56.6, 57.0, 67.2, 72.2, 109.5, 120.1, 122.4, 127.0, 127.5, 128.5, 129.1, 131.7, 142.3, 143.8, 158.0, 158.2, 161.2, 162.6; ES-MS m/z 597 (M+H). Anal. Calcd. for C₃₅H₄₀N₄O_{5.}0.8CH₂Cl₂: C, 64.69; H, 6.31; N, 8.43. Found: C, 64.45; H, 6.66; N, 8.18.

### EXAMPLE 88

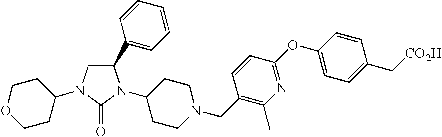

### COMPOUND 88: [4-(6-Methyl-5-{4-[(R)-2-oxo-5-phenyl-3-(tetrahydro-pyran-4-yl)-imidazolidin-1-yl]-piperidin-1-ylmethyl}-pyridin-2-yloxy)-phenyl]-acetic acid

A solution of 4-hydroxyphenylacetate (249 mg, 1.50 mmol), 6-chloro-2-methyl-pyridine-3-carbaldehyde (256 mg, 1.65 mmol) and K₂CO₃ (145 mg, 1.05 mmol) in DMF (3.0 mL) was heated to 130° C. for 1 hour. Aqueous work-up and purification afforded [4-(5-formyl-6-methyl-pyridin-2-yloxy)-phenyl]-acetic acid methyl ester (223 mg).

COMPOUND 88 was isolated as a pale pink foam (74 mg, 8% over 3 steps). ¹H NMR (CD₃OD) δ 1.50-1.87 (m, 7H), 2.21 (ddd, 1H, J=25.2, 12.6, 3.6 Hz), 2.43-2.59 (m, 5H), 3.05-3.09 (m, 1H), 3.15-3.20 (m, 2H), 3.48-3.63 (m, 5H), 3.78-3.84 (m, 3H), 3.92-4.04 (m, 3H), 4.77 (dd, 1H, J=9.3, 6.9 Hz), 6.67 (d, 1H, J=8.4 Hz), 7.04-7.07 (m, 2H), 7.35-7.43 (m, 7H), 7.68 (d, 1H, J=8.4 Hz); ¹³C NMR (CD₃OD) δ 21.68, 28.53, 29.57, 30.63, 31.01, 42.23, 50.30, 52.06, 53.28, 53.46, 57.84, 58.08, 68.01, 68.10, 109.10, 121.60, 123.22, 127.92, 129.42, 129.97, 131.74, 133.54, 143.14, 144.21, 154.20, 158.52, 161.60, 164.27, 176.45; ES-MS m/z 585 (M+1). Anal. Calcd. for C₃₄H₄₀N₄O₅.0.4CH₂Cl_{2.}0.6H₂O: C, 65.64; H, 6.72; N, 8.90. Found: C, 65.62; H, 6.73; N, 8.88.

### EXAMPLE 89

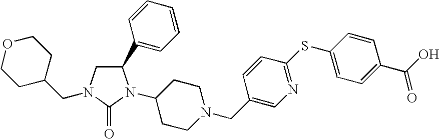

### COMPOUND 89: 4-(5-{4-[(R)-2-Oxo-5-phenyl-3-(tetrahydro-pyran-4-ylmethyl)-imidazolidin-1-yl]-piperidin-1-ylmethyl}-pyridin-2-ylsulfanyl)-benzoic acid

COMPOUND 89 was isolated as a colourless foam (166 mg, 75% over 2 steps). ¹H NMR (CD₃OD) δ 1.25-1.44 (m, 2H), 1.61-1.94 (m, 6H), 2.28 (ddd, 1H, J=25.5, 12.9, 3.6 Hz), 2.64-2.75 (m, 2H), 3.12-3.46 (m, 7H), 3.54-3.63 (m, 1H), 3.83 (t, 1H, J=9.3 Hz), 3.96-4.05 (m, 4H), 4.79 (dd, 1H, J=9.6, 7.2 Hz), 7.16 (d, 1H, J=8.4 Hz), 7.35-7.47 (m, 5H), 7.65 (d, 2H, J=8.4 Hz), 7.70 (dd, 1H, J=8.4, 2.4 Hz), 8.09 (d, 2H, J=8.4 Hz), 8.41 (d, 1H, J=1.8 Hz); ES-MS m/z 587 (M+1). Anal. Calcd. for C₃₃H₃₈N₄SO₄.0.4CH₂Cl₂1.4H₂O: C, 62.11; H, 6.49; N, 8.67. Found: C, 62.31; H, 6.48; N, 8.81.

### EXAMPLE 90

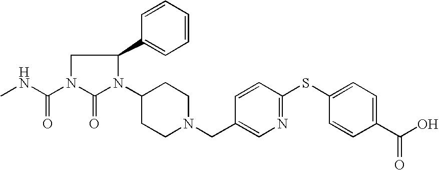

### COMPOUND 90: 4-{5-[4-((R)-3-Methylcarbamoyl-2-oxo-5-phenyl-imidazolidin-1-yl)-piperidin-1-ylmethyl]-pyridin-2-ylsulfanyl}-benzoic acid

COMPOUND 90 was isolated as a white powder (57.4 mg, 67% over 2 steps). ¹H NMR (CD₃OD) δ 1.61-1.70 (m, 2H), 1.82 (d, 1H, J=11.1 Hz), 2.21-2.35 (m, 1H), 2.46-2.57 (m, 2H), 2.86 (s, 3H), 3.12 (d, 1H, J=11.7 Hz), 3.22 (d, 1H, J=11.1 Hz), 3.57 (dd, 1H, J=10.5, 5.7 Hz), 3.63 (m, 1H), 3.86 (s, 2H), 4.17 (t, 1H, J=10.2 Hz), 4.83 (dd, 1H, J=9.6, 5.7 Hz), 7.05 (d, 1H, J=8.1 Hz), 7.31-7.38 (m, 5H), 7.61 (d, 2H, J=7.5 Hz), 7.67 (dd, 1H, J=8.4, 1.8 Hz), 8.05 (d, 1H, J=7.5 Hz), 8.38 (s, 1H); ES-MS m/z 546 (M+H). Anal. Calcd. for C₂₉H₃₁N₅O₄S.0.5CH₂Cl₂: C, 60.25; H, 5.48; N, 11.91. Found: C, 60.05; H, 5.56; N, 12.05.

### EXAMPLE 91

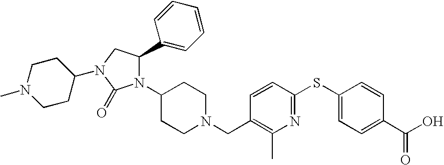

### COMPOUND 91: 4-(6-Methyl-5-{4-[(R)-3-(1-methyl-piperidin-4-yl)-2-oxo-5-phenyl-imidazolidin-1-yl]-piperidin-1-ylmethyl}-pyridin-2-ylsulfanyl)-benzoic acid

Following General Procedure A, ((R)-2-amino-1-phenyl-ethyl)-carbamic acid tert-butyl ester (1.15 g, 4.91 mmol) and 1-methylpiperidine (0.679 g, 6.00 mmol) gave the crude intermediate. The crude product was treated with TFA in CH₂Cl₂ following General Procedure C. The product was purified by flash chromatography on silica gel (CH₂Cl₂/MeOH/NH₄OH, 20:1:1 v/v/v) to give an oil (0.25 g, 22% two steps).

Following general procedure A, the product (0.25 g, 1.1 mmol) obtained in the last step and 1-boc-4-piperidone (0.30 g, 1.5 mmol) gave the desired carbamate. The crude product was dissolved in CH₂Cl₂ (5 mL) and cooled in an ice bath. To the cooled solution was added DIPEA (0.26 g, 2.0 mmol) and triphosgene (0.15 g, 0.51 mmol). The mixture was warmed to room temperature and stirred for 1 h. A saturated aqueous NaHCO₃ solution (15 mL) was added, and the mixture was extracted with CH₂Cl₂ (3×15 mL). The combined extract was dried over anhydrous Na₂SO₄. After filtration the solvent was removed, and the residue was purified by flash chromatography on silica gel (CH₂Cl₂/MeOH/NH₄OH, 100:5:1 v/v/v) to give 4-[(R)-3-(1-methyl-piperidin-4-yl)-2-oxo-5-phenyl-imidazolidin-1-yl]-piperidine-1-carboxylic acid tert-butyl ester as an oil (0.22 g, 46% two steps). ¹H NMR (CDCl₃) δ 1.35-1.44 (m, 11H), 1.65-1.72 (m, 5H), 2.05-2.08 (m, 2H), 2.26 (s, 2H), 2.85-2.90 (m, 2H), 3.04-3.09 (m, 2H), 3.07 (dd, 1H, J=9.0, 7.2 Hz), 3.63 (t, 1H, J=9.0 Hz), 3.76-3.87 (m, 3H), 4.09 (br s, 1H), 4.53 (dd, 1H, J=9.0, 6.9 Hz), 7.27-7.36 (m, 5H).

Following General Procedure C: BOC-deprotection was carried out to afford crude (R)-1-(1-methyl-piperidin-4-yl)-4-phenyl-3-piperidin-4-yl-imidazolidin-2-one.

COMPOUND 91 was isolated as a white foam (0.068 g, 22% over 3 steps). ¹H NMR (CD₃OD) δ 1.55-1.63 (m, 2H), 1.75-1.80 (m, 1H), 2.00-2.24 (m, 5H), 2.46-2.58 (m, 5H), 2.89 (s, 3H), 3.02-3.07 (m, 1H), 3.16-3.21 (m, 4H), 3.59-3.64 (m, 3H), 3.81-3.86 (m, 3H), 3.96-4.02 (m, 1H), 4.78-4.84 (m, 1H), 6.90 (d, 1H, J=8.1 Hz), 7.35-7.43 (m, 5H), 7.58-7.61 (m, 3H), 8.04-8.07 (m, 2H); ¹³C NMR (CD₃OD) δ 22.27, 27.45, 27.72, 28.71, 29.61, 43.73, 52.18, 53.60, 53.78, 54.82, 54.89, 58.05, 58.43, 121.33, 125.61, 128.21, 129.73, 130.28, 131.83, 134.69, 135.96, 136.90, 141.82, 143.21, 160.27, 161.11, 161.73, 171.60; ES-MS m/z 600 (M+H).

### EXAMPLE 92

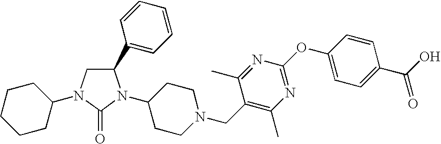

### COMPOUND 92: 4-{5-[4-((R)-3-Cyclohexyl-2-oxo-5-phenyl-imidazolidin-1-yl)-piperidin-1-ylmethyl]-4,6-dimethyl-pyrimidin-2-yloxy}-benzoic acid

To a solution of 4,6-dimethyl-2-hydroxypyrimidine (1.416 g, 11.42 mmol) in H₂O (20 mL) at 0° C. was added Br₂ (0.644 mL, 12.6 mmol). The mixture was warmed to room temperature, filtered and the filtrant was concentrated under reduced pressure to afford crude 5-bromo-4,6-dimethyl-pyrimidin-2-ol. A mixture of the bromide and POCl₃ (12 mL) was heated at reflux overnight. Basic work-up and purification afforded crude 5-bromo-2-chloro-4,6-dimethyl-pyrimidine (717 mg). To a solution of methyl 4-hydroxybenzoate (495 mg, 3.24 mmol) in DMF (20 mL) was added NaH (60%, 155 mg, 3.89 mmol) and the mixture was stirred at room temperature for 30 minutes. The above chloride (717 mg) was added and the mixture was heated to 80° C. for 30 minutes and at 120° C. for 2 hours. Standard work-up and purification afforded 4-(5-bromo-4,6-dimethyl-pyrimidin-2-yloxy)-benzoic acid methyl ester (304 mg, 8% over 2 steps).

A mixture of the bromide (304 mg, 0.902 mmol), tributyl(vinyl)tin (0.315 mL, 1.08 mmol) and bis(triphenylphosphine)palladium(II) dichloride (94 mg, 0.13 mmol) in degassed DMF (15 mL) was heated to 80° C. overnight. The mixture was concentrated under reduced pressure and the resulting solid was washed with hexanes and dried in vacuo to afford 4-(4,6-dimethyl-5-vinyl-pyrimidin-2-yloxy)-benzoic acid methyl ester (171 mg, 67%).

To AD-mix-α (820 mg) was added a solution of tert-butanol (3.3 mL) and H₂O (0.6 mL) and the mixture was stirred at room temperature for 15 minutes. A solution of the above substrate (171 mg, 0.602 mmol) in THF (1.0 mL) was added followed by OsO₄ (2.5%, 0.12 mL) and the mixture stirred at room temperature overnight. Standard work-up afforded the desired intermediate. To a solution of the diol in acetone (4 mL) was added a solution of NaIO₄ (256 mg, 1.20 mmol) in H₂O (2 mL) and the mixture was stirred at room temperature for 2 hours. Aqueous work-up and purification afforded 4-(5-formyl-4,6-dimethyl-pyrimidin-2-yloxy)-benzoic acid methyl ester (66 mg, 38% over 2 steps).

COMPOUND 92 was isolated as a white solid (66 mg, 49% over 2 steps). ¹H NMR (CD₃OD) δ 1.14-1.52 (m, 6H), 1.67-1.91 (m, 5H), 1.95-2.06 (m, 1H), 2.19-2.33 (m, 2H), 2.47 (s, 6H), 2.79-2.83 (m, 1H), 2.94-2.98 (m, 1H), 3.12-3.18 (m, 1H), 3.51-3.81 (m, 5H), 4.66-4.84 (m, 3H), 7.26-7.29 (m, 2H), 7.36-7.41 (m, 5H), 8.09-8.12 (m, 2H); ES-MS m/z 584 (M+1). Anal. Calcd. for C₃₄H₄₁N₅O₄.0.08CH₂Cl₂.0.8CH₄O: C, 68.02; H, 7.26; N, 11.37. Found: C, 68.03; H, 7.23; N, 11.32.

### EXAMPLE 93

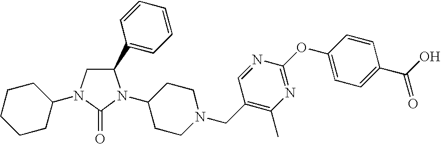

### COMPOUND 93: 4-{5-[4-((R)-3-Cyclohexyl-2-oxo-5-phenyl-imidazolidin-1-yl)-piperidin-1-ylmethyl]-4-methyl-pyrimidin-2-yloxy}-benzoic acid

A solution of acetylacetaldehyde dimethyl acetal (5.091 g, 38.52 mmol), urea (2.300 g, 38.52 mmol), conc. HCl (5 mL) and EtOH (20 mL) was heated to 85° C. overnight. Standard work-up afforded the pyrimidine. To a solution of the pyrimidine in H₂O (61 mL) was added Br₂ (2 mL) slowly. The mixture was stirred at room temperature for 30 minutes and heated to 80° C. for an additional 30 minutes. The mixture was cooled to room temperature, filtered and the filtrant was concentrated under reduced pressure. H₂O was added, the mixture filtered and the filtrant was neutralized with 10N NaOH and concentrated under reduced pressure. A solution of the crude bromide in POCl₃ (excess) was heated at reflux overnight. Standard work-up and purification afforded 5-bromo-2-chloro-4-methyl-pyrimidine (790 mg, 10% over 3 steps).

To a solution of methyl 4-hydroxybenzoate (579 mg, 3.81 mmol) in DMF (20 mL) was added NaH (60%, 182 mg, 4.56 mmol) and the mixture was stirred at room temperature for 30 minutes. The above pyrimidine (790 mg, 3.81 mmol) was added and the mixture was heated to 60° C. for 30 minutes and at 120° C. for 2 hours. Standard work-up and purification afforded 4-(5-bromo-4-methyl-pyrimidin-2-yloxy)-benzoic acid methyl ester (362 mg, 29%).

A mixture of the bromide (362 mg, 1.12 mmol), tributyl(vinyl)tin (0.44 mL, 1.5 mmol) and bis(triphenylphosphine)palladium(II) dichloride (120 mg, 0.17 mmol) in degassed DMF (10 mL) was heated to 85° C. for 5 hours. Standard work-up and purification afforded 4-(4-methyl-5-vinyl-pyrimidin-2-yloxy)-benzoic acid methyl ester (303 mg, quant).

To AD-mix-α (1.6 g) was added a solution of tert-butanol (5 mL) and H₂O (5 mL) and the mixture was stirred at room temperature for 15 minutes. A solution of the above substrate (303 mg, 1.12 mmol) in THF (0.5 mL) was added followed by OsO₄ (2.5%, 0.1 mL) and the mixture stirred at room temperature overnight. A second aliquot of OsO₄ (0.1 mL) was added and the mixture stirred overnight. Standard work-up afforded the desired intermediate (240 mg). To a solution of the diol in acetone (4 mL) was added a solution of NaIO₄ (336 mg, 1.58 mmol) in H₂O (2 mL) and the mixture was stirred at room temperature for 2 hours. Aqueous work-up and purification afforded 4-(5-formyl-4-methyl-pyrimidin-2-yloxy)-benzoic acid methyl ester (174 mg, 57% over 2 steps).

COMPOUND 93 was isolated as a white solid (79 mg, 63% over 2 steps). ¹H NMR (CDCl₃) δ 1.00-1.07 (m, 1H), 1.25-1.85 (m, 13H), 2.36-2.76 (m, 5H), 3.09-3.14 (m, 2H), 3.39 (br s, 1H), 3.65-3.97 (m, 5H), 4.56-4.60 (m, 1H), 7.17-7.30 (m, 7H), 8.02 (d, 2H, J=8.1 Hz), 8.57 (br s, 1H); ¹³C NMR (CDCl₃) δ 23.01, 25.83, 25.92, 26.77, 29.50, 30.41, 30.68, 48.76, 50.11, 51.91, 52.61, 54.78, 55.90, 121.86, 127.13, 128.07, 128.79, 129.42, 132.06, 142.72, 156.80, 160.34, 162.58, 164.76, 168.93, 171.56; ES-MS m/z 570 (M+1).

### EXAMPLE 94

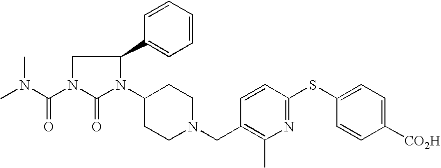

### COMPOUND 94: 4-{5-[4-((R)-3-Dimethylcarbamoyl-2-oxo-5-phenyl-imidazolidin-1-yl)-piperidin-1-ylmethyl]-6-methyl-pyridin-2-ylsulfanyl}-benzoic acid

COMPOUND 94 was isolated as a white foam (98.9 mg, 83% over 2 steps). ¹H NMR (CDCl₃) δ 1.54 (br s, 1H), 1.71 (br s, 1H), 1.86 (br s, 1H), 2.40 (br s, 2H), 2.52 (s, 3H), 3.04 (s, 6H), 3.10 (m, 2H), 3.40-3.45 (m, 2H), 3.84-3.91 (m, 3H), 4.15 (t, 1H, J=9.3 Hz), 4.68 (br s, 1H), 6.60 (br s, 1H), 7.26 (m, 5H), 7.59 (d, 2H, J=7.5 Hz), 7.67 (br s, 1H), 7.99 (d, 2H, J=7.5 Hz); ¹³C NMR (CDCl₃) δ 21.05, 25.59, 26.66, 36.56, 48.68, 49.88, 50.50, 53.60, 55.23, 118.11, 121.21, 125.02, 127.28, 127.75, 129.52, 131.49, 132.83, 134.29, 138.85, 139.60, 154.07, 154.68, 156.40, 159.46, 167.88; ES-MS m/z 574 (M+H); Anal. Calcd. for C₃₁H₃₅N₅O₄S.2.0CH₂Cl₂: C, 53.31; H, 5.29; N, 9.42. Found: C, 53.05; H, 5.15; N, 9.49.

### EXAMPLE 95

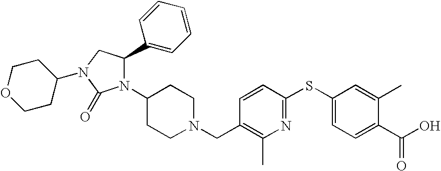

### COMPOUND 95: 2-Methyl-4-(6-methyl-5-{4-[(R)-2-oxo-5-phenyl-3-(tetrahydro-pyran-4-yl)-imidazolidin-1-yl]-piperidin-1-ylmethyl}-pyridin-2-ylsulfanyl)-benzoic acid

Following the procedure as described for 6-(4-bromo-2-methyl-phenylsulfanyl)-2-methyl-pyridine-3-carbaldehyde (see EXAMPLE 108) using 4-bromo-3-methylaniline (5.40 g, 29.0 mmol), 6-chloro-2-methylpyridine-3-carboxaldehyde (2.60 g, 16.7 mmol) and K₂CO₃ (3.00 g, 21.7 mmol). The product was purified by flash chromatography on silica gel (EtOAc/hexanes, 1:4 in v/v) to afford 6-(4-bromo-3-methyl-phenylsulfanyl)-2-methyl-pyridine-3-carbaldehyde as a pale yellow oil (4.46 g, 83%). ¹H NMR (CDCl₃) δ 2.43 (s, 3H), 2.81 (s, 3H), 6.75 (d, 1H, J=8.4 Hz), 7.29 (dd, 1H, J=8.1, 2.1 Hz), 7.48 (d, 1H, J=2.1 Hz), 7.63 (d, 1H, J=8.1 Hz), 7.83 (d, 1H, J=8.4 Hz), 10.21 (s, 1H).

A mixture of 6-(4-bromo-3-methyl-phenylsulfanyl)-2-methyl-pyridine-3-carbaldehyde (2.80 g, 8.33 mmol) and NaBH(OAc)₃ (4.00 g, 18.9 mmol) in CH₂Cl₂ (50 mL) was stirred at room temperature for 24 h. Saturated aqueous NaHCO₃ (50 mL) and brine (50 mL) were added and the mixture was extracted with CH₂Cl₂ (3×60 mL). The combined extract was dried over anhydrous Na₂SO₄. After filtration the solvent was removed, and the residue was purified by flash chromatography on silica gel (EtOAc/hexanes 1:1 v/v) to afford [6-(4-bromo-3-methyl-phenylsulfanyl)-2-methyl-pyridin-3-yl]-methanol as a pale yellow solid (2.0 g, 71%). ¹H NMR (CDCl₃) δ 2.40 (s, 3H), 2.51 (s, 3H), 4.66 (s, 2H), 6.73 (d, 1H, J=8.1 Hz), 7.25 (dd, 1H, J=8.1, 2.1 Hz), 7.44 (d, 1H, J=2.1 Hz), 7.47 (d, 1H, J=8.1 Hz), 7.55 (d, 1H, J=8.1 Hz).

At 0° C., to a solution of [6-(4-bromo-3-methyl-phenylsulfanyl)-2-methyl-pyridin-3-yl]-methanol (2.00 g, 6.17 mmol) in anhydrous THF (30 mL) was added NaH (60% in mineral oil, 0.48 g, 12 mmol). The mixture was stirred at 0° C. for 10 min, then at room temperature for 30 min. CH₃OCH₂Cl (0.805 g, 10.0 mmol) was then added and the mixture was stirred for 16 h. Saturated aqueous NaHCO₃ (20 mL) and brine (20 mL) were added and the mixture was extracted with CH₂Cl₂ (4×40 mL). The combined extract was dried over anhydrous Na₂SO₄. After filtration the solvent was removed, and the residue was purified by flash chromatography on silica gel (EtOAc/hexanes 1:5 v/v) to afford 6-(4-bromo-3-methyl-phenylsulfanyl)-3-methoxymethoxymethyl-2-methyl-pyridine as a pale yellow solid (1.84 g, 81%). ¹H NMR (CDCl₃) δ 2.40 (s, 3H), 2.52 (s, 3H), 3.39 (s, 3H), 4.53 (s, 2H), 4.69 (s, 2H), 6.71 (d, 1H, J=8.1 Hz), 7.25 (dd, 1H, J=8.1, 2.1 Hz), 7.42-7.45 (m, 2H), 7.54 (d, 1H, J=8.1 Hz).

At −78° C., to a solution 6-(4-bromo-3-methyl-phenylsulfanyl)-3-methoxymethoxymethyl-2-methyl-pyridine (1.84 g, 5.00 mmol) in anhydrous THF (30 mL) was added tert-BuLi (1.7 M in pentane, 4.4 mL, 7.5 mL). After addition the mixture was stirred at −78° C. for 15 min, and CO₂ was introduced. After bubbling for 20 min, water (20 mL) was added and the mixture was acidified carefully with 1 N HCl. Extraction with CH₂Cl₂ (10×20 mL) was performed and the combined extracts were dried over anhydrous Na₂SO₄. After filtration the solvent was removed and the residue was dissolved in DMF (15 mL). MeI (1.0 mL, 15 mmol) and K₂CO₃ (1.38 g, 10.0 mmol) were added and the mixture was stirred at room temperature for 5 h. After concentration saturated aqueous NH₄Cl (20 mL) and brine (20 mL) were added and the mixture was extracted with CH₂Cl₂ (4×40 mL). The combined extract was dried over anhydrous Na₂SO₄. After filtration the solvent was removed, and the residue was purified by flash chromatography on silica gel (EtOAc/hexanes 1:4 v/v) to afford 4-(5-methoxymethoxymethyl-6-methyl-pyridin-2-ylsulfanyl)-2-methyl-benzoic acid methyl ester as a pale yellow oil (0.694 g, 40%). ¹H NMR (CDCl₃) δ 2.53 (s, 3H), 2.58 (s, 3H), 3.40 (s, 3H), 4.55 (s, 2H), 4.71 (s, 2H), 6.88 (d, 1H, J=8.1 Hz), 7.35 (dd, 1H, J=8.1, 1.5 Hz), 7.40 (d, 1H, J=1.5 Hz), 7.49 (d, 1H, J=8.1 Hz), 7.89 (d, 1H, J=8.1 Hz).

4-(5-Methoxymethoxymethyl-6-methyl-pyridin-2-ylsulfanyl)-2-methyl-benzoic acid methyl ester (0.694 g, 2.00 mmol) was stirred in aqueous HCl (6 N, 5 mL) and methanol (5 mL) for 30 min. Saturated aqueous NaHCO₃ (20 mL) was added and the mixture was extracted with CH₂Cl₂ (4×40 mL). The combined extract was dried over anhydrous Na₂SO₄. After filtration the solvent was removed, and the residue was purified by flash chromatography on silica gel (EtOAc/hexanes 1:1 v/v) to afford 4-(5-hydroxymethyl-6-methyl-pyridin-2-ylsulfanyl)-2-methyl-benzoic acid methyl ester as a pale yellow solid (0.490 g, 78%). ¹H NMR (CDCl₃) δ 2.51 (s, 3H), 2.57 (s, 3H), 3.89 (s, 3H), 4.68 (s, 2H), 6.89 (d, 1H, J=8.1 Hz), 7.35 (dd, 1H, J=8.1, 1.5 Hz), 7.39 (d, 1H, J=1.5 Hz), 7.53 (d, 1H, J=8.1 Hz), 7.89 (d, 1H, J=8.1 Hz).

4-(5-Hydroxymethyl-6-methyl-pyridin-2-ylsulfanyl)-2-methyl-benzoic acid methyl ester (0.490 g, 1.56 mmol) and MnO₂ (3 g) were stirred in CH₂Cl₂ (30 mL) for 16 h. The suspension was then filtered through a Celite® cake and the solvent was removed to afford 4-(5-formyl-6-methyl-pyridin-2-ylsulfanyl)-2-methyl-benzoic acid methyl ester as a pale yellow solid (0.380 g, 78%). ¹H NMR (CDCl₃) δ 2.62 (s, 3H), 2.81 (s, 3H), 3.93 (s, 3H), 6.82 (d, 1H, J=8.1 Hz), 7.46-7.51 (m, 2H), 7.84 (d, 1H, J=8.4 Hz), 7.97 (d, 1H, J=8.1 Hz), 10.22 (s, 1H).

COMPOUND 95 was isolated as a brown solid (0.430 g, 71% over 2 steps). ¹H NMR (CD₃OD) δ 1.45-1.81 (m, 7H), 2.11-2.16 (m, 1H), 2.28-2.41 (m, 2H), 2.51 (s, 3H), 2.56 (s, 3H), 2.91-2.96 (m, 2H), 3.04-3.09 (m, 2H), 3.16 (dd, 1H, J=8.7, 6.6 Hz), 3.47-3.58 (m, 3H), 3.66 (s, 2H), 3.80 (t, 1H, J=9.3 Hz), 3.95-4.03 (m, 3H), 4.76 (dd, 1H, J=9.3, 6.6 Hz), 6.82 (d, 1H, J=8.1 Hz), 7.33-7.42 (m, 7H), 7.51 (d, 1H, J=8.1 Hz), 7.80 (d, 1H, J=7.8 Hz); ¹³C NMR (CD₃OD) δ 20.88, 21.59, 22.23, 28.81, 30.05, 30.91, 31.22, 50.48, 52.37, 53.48, 53.73, 55.03, 57.79, 58.44, 68.21, 68.29, 120.48, 125.93, 128.05, 129.58, 130.17, 131.74, 132.53, 133.87, 134.62, 137.72, 138.02, 140.59, 141.40, 143.52, 159.77, 161.54, 174.58; ES-MS m/z 601 (M+H). Anal. Calcd. for C₃₄H₄₀N₄O₄S.0.2CH₂Cl₂.1.2H₂O: C, 65.73; H, 6.64, N, 8.96; S, 5.13. Found: C, 65.47; H, 6.63; N, 8.88; S, 5.04.

### EXAMPLE 96

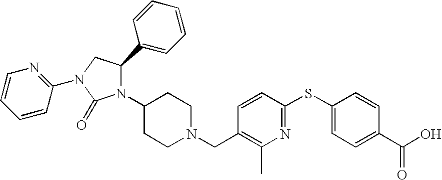

### COMPOUND 96: 4-{6-Methyl-5-[4-((R)-2-oxo-5-phenyl-3-pyridin-2-yl-imidazolidin-1-yl)-piperidin-1-ylmethyl]-pyridin-2-ylsulfanyl}-benzoic acid

To an Argon-purged suspension of 4-((R)-2-oxo-5-phenyl-imidazolidin-1-yl)-piperidine-1-carboxylic acid tert-butyl ester (208 mg, 0.60 mmol), 2-bromopyridine (0.10 mL, 1.05 mmol) and Cs₂CO₃ (249 mg, 0.76 mmol) in dioxane (4 mL) was added catalytic 4,5-bis(diphenylphosphino)-9,9-dimethylxanthene (11 mg, 0.019 mmol) and Pd₂(dba)₃ (10 mg, 0.011 mmol) and the reaction stirred at 100° C. overnight. Aqueous work-up and purification afforded 4-((R)-2-oxo-5-phenyl-3-pyridin-2-yl-imidazolidin-1-yl)-piperidine-1-carboxylic acid tert-butyl ester (0.11 g, 43%) as a clear oil.

Following general procedure C: a solution of the Boc-protected material from above (0.11 g, 0.26 mmol) in CH₂Cl₂/TFA (2:1, 4.5 mL) was stirred at room temperature for 5 h then worked up as usual to give (R)-4-phenyl-3-piperidin-4-yl-1-pyridin-2-yl-imidazolidin-2-one (108 mg) as a yellow oil.

COMPOUND 96 was isolated as a white solid (51 mg, 26% over 2 steps). ¹H NMR (CD₃OD) δ 1.71-1.90 (m, 3H), 2.28-2.34 (m, 1H), 2.51 (s, 3H), 2.69-2.76 (m, 2H), 3.15-3.31 (m, 2H), 3.66-3.83 (m, 2H), 3.95 (s, 2H), 4.40 (t, 1H, J=10 Hz), 4.86-4.92 (m, 1H), 6.91 (d, 1H, J=8.1 Hz), 6.98-7.02 (m, 1H), 7.33-7.44 (m, 5H), 7.57-7.61 (m, 3H), 7.72 (t, 1H, J=7.8 Hz), 8.04 (d, 2H, J=7.5 Hz), 8.24 (d, 2H, J=7.5 Hz); ¹³C NMR (CDCl₃) δ 22.47, 26.53, 27.88, 29.62, 49.96, 51.16, 51.84, 54.71, 56.38, 112.96, 117.89, 119.51, 121.97, 126.54, 128.56, 129.09, 130.90, 134.14, 135.71, 137.34, 140.36, 141.63, 147.33, 152.07, 156.79, 157.80, 160.96, 168.81; ES-MS m/z 580 (M+H). Anal. Calcd. for C₃₃H₃₃N₅O₃S.1.3CH₂Cl₂: C, 59.70; H, 5.20; N, 10.15. Found: C, 59.57; H, 5.17; N, 10.08.

### EXAMPLE 97

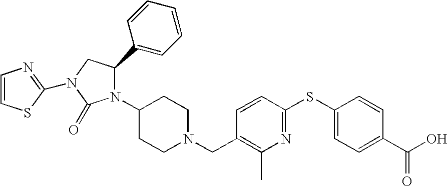

### COMPOUND 97: 4-{6-Methyl-5-[4-((R)-2-oxo-5-phenyl-3-thiazol-2-yl-imidazolidin-1-yl)-piperidin-1-ylmethyl]-pyridin-2-ylsulfanyl}-benzoic acid

To an Argon-purged suspension of 4-((R)-2-oxo-5-phenyl-imidazolidin-1-yl)-piperidine-1-carboxylic acid tert-butyl ester (224 mg, 0.65 mmol), 2-bromothiazole (70 μL, 0.79 mmol) and Cs₂CO₃ (276 mg, 0.85 mmol) in dioxane (5 mL) was added catalytic 4,5-bis(diphenylphosphino)-9,9-dimethylxanthene (15 mg, 0.026 mmol) and Pd₂(dba)₃ (11 mg, 0.012 mmol) and the reaction stirred at 100° C. overnight. Aqueous work-up and purification afforded 4-((R)-2-oxo-5-phenyl-3-thiazol-2-yl-imidazolidin-1-yl)-piperidine-1-carboxylic acid tert-butyl ester (0.23 g, 83%) as a white foam.

Following general procedure C: a solution of the Boc-protected material from above (0.23 g, 0.54 mmol) in CH₂Cl₂/TFA (2:1, 4.5 mL) was stirred at room temperature for 2 h then worked up as usual to give (R)-4-phenyl-3-piperidin-4-yl-1-thiazol-2-yl-imidazolidin-2-one (178 mg) as a white foam.

COMPOUND 97 was isolated as a white solid (74 mg, 43% over 2 steps). ¹H NMR (CDCl₃) δ 1.52-1.62 (m, 2H), 2.31-2.43 (m, 3H), 2.50 (s, 3H), 2.60-2.68 (m, 2H), 2.98-3.05 (m, 1H), 3.20-3.28 (m, 1H), 3.71 (br s, 2H), 3.90-3.95 (m, 1H), 4.42 (t, 1H, J=10 Hz), 4.82-4.87 (m, 1H), 6.65 (d, 1H, J=7.2 Hz), 6.90 (d, 1H, J=3.6 Hz), 7.26-7.30 (m, 5H), 7.33 (d, 1H, J=3.6 Hz), 7.50-7.55 (m, 1H), 7.57 (d, 2H, J=7.2 Hz), 8.00 (d, 2H, J=7.2 Hz); ¹³C NMR (CDCl₃+2 drops CD₃OD) δ 22.77, 27.24, 28.41, 30.07, 50.95, 52.47, 56.37, 57.04, 113.12, 120.13, 122.98, 127.06, 129.39, 129.72, 131.33, 132.70, 134.36, 136.45, 137.84, 140.68, 141.09, 156.04, 158.40, 159.49, 160.97, 169.30; ES-MS m/z 586 (M+H). Anal. Calcd. for C₃₁H₃₁N₅O₃S₂.0.8CH₂Cl₂: C, 58.43; H, 5.03; N, 10.71. Found: C, 58.50; H, 5.27; N, 10.32.

### EXAMPLE 98

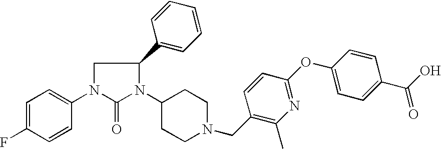

### COMPOUND 98: 4-(5-{4-[(R)-3-(4-Fluoro-phenyl)-2-oxo-5-phenyl-imidazolidin-1-yl]-piperidin-1-ylmethyl}-6-methyl-pyridin-2-yloxy)-benzoic acid

See EXAMPLE 77 for the preparation of (R)-1-(4-fluoro-phenyl)-4-phenyl-3-piperidin-4-yl-imidazolidin-2-one. COMPOUND 98 was isolated as a white foam (77 mg, 32% over 2 steps). ¹H NMR (CD₃OD) δ 1.46-1.63 (m, 2H), 1.75-1.79 (m, 1H), 2.10-2.32 (m, 3H), 2.41 (s, 3H), 2.88-2.91 (m, 1H), 3.02-3.06 (m, 1H), 3.57-3.68 (m, 5H), 4.24 (t, 1H, J=9.3 Hz), 6.75 (d, 1H, J=8.4 Hz), 7.03-7.12 (m, 4H), 7.29-7.46 (m, 5H), 7.50-7.55 (m, 2H), 7.69 (d, 1H, J=8.1 Hz), 8.04 (d, 2H, J=8.7 Hz).

### EXAMPLE 99

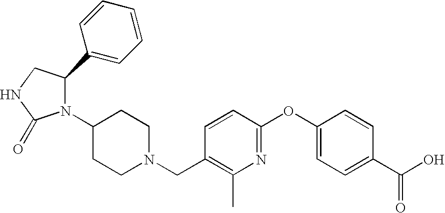

### COMPOUND 99: 4-{6-Methyl-5-[4-((R)-2-oxo-5-phenyl-imidazolidin-1-yl)-piperidin-1-ylmethyl]-pyridin-2-yloxy}-benzoic acid

COMPOUND 99 was isolated as a white powder (159 mg, 62% over 2 steps). ¹H NMR (CD₃OD) δ 1.75-1.95 (m, 3H), 2.30-2.40 (m, 1H), 2.45 (s, 3H), 2.90-3.10 (m, 2H), 3.15-3.23 (m, 1H), 3.36-3.38 (m, 1H), 3.44 (d, 1H, J=12.0 Hz), 3.59 (m, 1H), 3.77 (t, 1H, J=9.3 Hz), 4.22 (s, 2H), 4.81-4.84 (m, 1H), 6.89 (d, 1H, J=8.4 Hz), 7.18 (d, 2H, J=8.7 Hz), 7.25-7.45 (m, 5H), 7.81-7.87 (m, 1H), 8.06 (d, 2H, J=8.7 Hz); ¹³C NMR (CD₃OD) δ 20.8, 26.0, 26.7, 48.9, 51.4, 51.5, 55.6, 58.8, 109.2, 119.0, 119.9, 126.5, 128.0, 128.6, 131.1, 141.5, 144.0, 157.6, 157.9, 162.4; ES-MS m/z 487 (M+H). Anal. Calcd. for C₂₈H₃₀N₄O₄.0.6CH₂Cl₂.1.6H₂O: C, 60.65; H, 6.12; N, 9.89. Found: C, 60.83; H, 6.14; N, 9.96.

### EXAMPLE 100

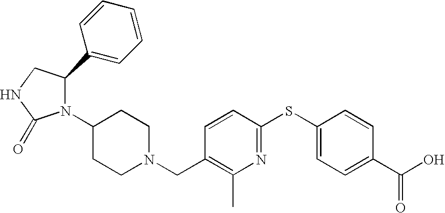

### COMPOUND 100: 4-{6-Methyl-5-[4-((R)-2-oxo-5-phenyl-imidazolidin-1-yl)-piperidin-1-ylmethyl]-pyridin-2-ylsulfanyl}-benzoic acid

COMPOUND 100 was isolated as a white powder (149 mg, 63% over 2 steps). ¹H NMR (CD₃OD) δ 1.75 (br s, 2H), 1.84 (d, 1H, J=13.2 Hz), 2.33-2.41 (m, 1H), 2.52 (s, 3H), 2.80-2.96 (m, 2H), 3.18 (t, 1H, J=7.8 Hz), 3.26-3.41 (m, 2H), 3.61 (br s, 1H), 3.76 (t, 1H, J=9.0 Hz), 4.13 (s, 2H), 4.82 (t, 1H, J=7.8 Hz), 4.88 (m, 1H), 6.88 (d, 1H, J=7.8 Hz), 7.30-7.43 (m, 5H), 7.60 (d, 2H, J=7.2 Hz), 7.65 (d, 1H, J=7.8 Hz), 8.02 (d, 2H, J=7.2 Hz); ¹³C NMR (CD₃OD) δ 23.0, 28.2, 29.0, 51.1, 53.6, 53.7, 57.9, 60.8, 121.5, 123.2, 128.5, 130.0, 130.6, 132.3, 134.2, 135.3, 137.7, 142.8, 143.7, 160.8, 162.4, 164.8, 170.2; ES-MS m/z 503 (M+H). Anal. Calcd. for C₂₈H₃₀N₄O₃S.0.4CH₂Cl₂.1.4H₂O: C, 60.72; H, 6.03; N, 9.97. Found: C, 60.62; H, 6.04; N, 9.87.

### EXAMPLE 101

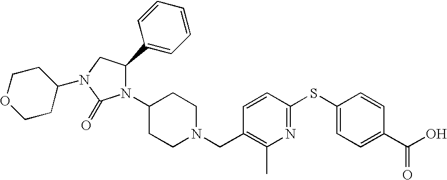

### COMPOUND 101: 4-(6-Methyl-5-{4-[(R)-2-oxo-5-phenyl-3-(tetrahydro-pyran-4-yl)-imidazolidin-1-yl]-piperidin-1-ylmethyl}-pyridin-2-ylsulfanyl)-benzoic acid

COMPOUND 101 was isolated as a white foam (0.065 g, 44% over 2 steps). ¹H NMR (CD₃OD) δ 1.62-1.81 (m, 7H), 2.19-2.25 (m, 1H), 2.50 (s, 3H), 2.56-2.68 (m, 2H), 3.11-3.32 (m, 4H), 3.43-3.56 (m, 3H), 3.77 (t, 1H, J=9.0 Hz), 3.89 (s, 2H), 3.90-3.99 (m, 3H), 4.88 (dd, 1H, J=9.3, 7.2 Hz), 6.90 (d, 1H, J=7.8 Hz), 7.31-7.38 (m, 5H), 7.54-7.60 (m, 3H), 8.02-8.05 (m, 2H); ¹³C NMR (CD₃OD) δ 22.25, 28.44, 29.38, 30.91, 31.31, 50.60, 51.89, 53.48, 53.74, 58.20, 68.30, 68.39, 121.35, 124.79, 128.21, 129.74, 130.28, 131.95, 134.15, 134.80, 137.67, 141.91, 143.26, 160.35, 161.37, 161.83, 170.14; ES-MS m/z 605 (M+H). Anal. Calcd. for C₃₃H₃₈N₄O₄S.0.1CH₂Cl₂.1.6H₂O: C, 63.71; H, 6.69; N, 8.98; S, 5.14. Found: C, 63.68; H, 6.77; N, 8.74; S, 5.00.

Examples 102 to 108 were prepared following the scheme illustrated below. RCHO is as defined in the table and X is as defined in the individual examples.

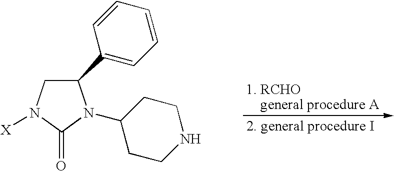

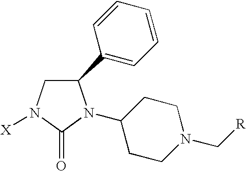

| Example | RCHO |
|---|---|
| 102 | 4-(5-formyl-6-methyl-pyridin-2-yloxy)-3-methyl-benzonitrile |
| 103 | 4-(5-formyl-6-methyl-pyridin-2-yloxy)-2-methyl-benzonitrile |
| 104 | 4-(5-formyl-6-methyl-pyridin-2-yloxy)-3-methyl-benzonitrile |
| 105 | 3-fluoro-4-(5-formyl-6-methyl-pyridin-2-yloxy)-benzonitrile |
| 106 | 4-(5-formyl-4-methyl-pyrimidin-2-ylamino)-benzonitrile |
| 107 | 4-(5-formyl-4-methyl-pyrimidin-2-ylamino)-benzonitrile |
| 108 | 4-(5-formyl-6-methyl-pyridin-2-ylsulfanyl)-3-methyl- |
| | benzonitrile |

### EXAMPLE 102

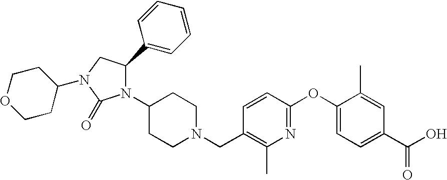

### COMPOUND 102: 3-Methyl-4-(6-methyl-5-{4-[(R)-2-oxo-5-phenyl-3-(tetrahydro-pyran-4-yl)-imidazolidin-1-yl]-piperidin-1-ylmethyl}-pyridin-2-yloxy)-benzoic acid

COMPOUND 102 was isolated as a white powder. (35.5 mg, 49% over 2 steps). ¹H NMR (CDCl₃) δ 1.46 (m, 1H), 1.65-1.81 (m, 5H), 2.22-2.43 (m, 2H), 2.24 (s, 3H), 2.41 (s, 3H), 3.00 (m, 1H), 3.11 (t, 1H, J=7.5 Hz), 3.30 (m, 1H), 3.44-3.53 (m, 2H), 3.67 (t, 2H, J=9.0 Hz), 3.63-4.10 (m, 7H), 4.61 (t, 1H, J=7.5 Hz), 6.50 (br s, 1H), 7.01 (d, 1H, J=8.1 Hz), 7.23-7.29 (m, 6H), 7.81 (d, 1H, J=8.1 Hz), 7.93 (s, 1H); ¹³C NMR (CDCl₃) δ 17.1, 23.0, 27.8, 29.5, 30.6, 30.8, 49.1, 49.5, 51.3, 53.0, 56.6, 57.7, 67.9, 68.0, 108.8, 121.3, 127.4, 128.9, 129.1, 129.7, 130.9, 134.0, 142.9, 143.8, 156.8, 157.7, 160.6, 162.8, 170.4; ES-MS m/z 585 (M+H). Anal. Calcd. for C₃₄H₄₀N₄O₅.1.1CH₂Cl₂: C, 62.17; H, 6.27; N, 8.26. Found: C, 62.14; H, 6.31; N, 8.03.

### EXAMPLE 103

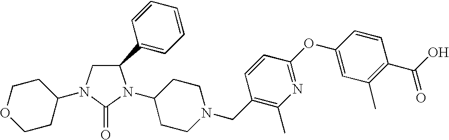

### COMPOUND 103: 2-Methyl-4-(6-methyl-5-{4-[(R)-2-oxo-5-phenyl-3-(tetrahydro-pyran-4-yl)-imidazolidin-1-yl]-piperidin-1-ylmethyl}-pyridin-2-yloxy)-benzoic acid

A solution of 4-bromo-3-methylphenol (255 mg, 1.36 mmol), 6-bromo-2-methyl-pyridine-3-carbaldehyde (298 mg, 1.49 mmol) and K₂CO₃ (226 mg, 1.64 mmol) in DMF (2.7 mL) was heated to 140° C. for 2 hours. Acidic work-up and purification afforded 6-(4-bromo-3-methyl-phenoxy)-2-methyl-pyridine-3-carbaldehyde (141 mg, 34%).

A mixture of the above bromide (110 mg, 0.359 mmol), Zn(CN)₂ (46 mg, 0.39 mmol), Pd₂(dba)₃ (33 mg, 0.036 mmol) and DPPF (40 mg, 0.072 mmol) in degassed DMF (1.5 mL) was heated to 140° C. for 12 hours. Aqueous work-up and purification afforded 4-(5-formyl-6-methyl-pyridin-2-yloxy)-2-methyl-benzonitrile (36 mg, 40%).

COMPOUND 103 was isolated as a yellow foam (33 mg, 41% over 2 steps). ¹H NMR (CD₃OD) δ 1.61-1.99 (m, 7H), 2.29-2.42 (m, 1H), 2.45 (s, 3H), 2.58 (s, 3H), 2.88-2.99 (m, 2H), 3.16 (t, 1H, J=7.8 Hz), 3.45-3.64 (m, 5H), 3.79 (t, 1H, J=9.0 Hz), 3.90-4.00 (m, 3H), 4.16 (s, 2H), 7.74 (dd, 1H, J=9.0, 7.5 Hz), 6.84 (d, 1H, J=8.1 Hz), 6.96-7.00 (m, 2H), 7.34-7.41 (m, 5H), 7.81 (d, 1H, J=8.4 Hz), 7.97 (d, 1H, J=8.4 Hz); ¹³C NMR (CDCl₃) δ 22.29, 22.46, 25.99, 27.81, 29.93, 30.02, 48.39, 48.87, 49.30, 51.79, 55.23, 67.09, 67.16, 109.67, 117.96, 123.61, 125.69, 126.66, 128.58, 129.19, 133.28, 142.05, 143.38, 144.17, 156.82, 157.15, 159.76, 162.61, 170.13; ES-MS m/z 585 (M+1). Anal. Calcd. for C₃₄H₄₀N₄O₅.0.8CH₂Cl₂.0.9CH₄O: C, 62.92; H, 6.68; N, 8.22. Found: C, 63.15; H, 6.65; N, 8.13.

### EXAMPLE 104

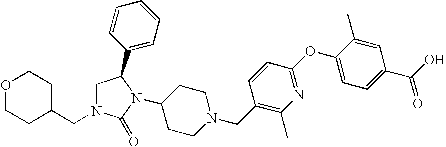

### COMPOUND 104: 3-Methyl-4-(6-methyl-5-{4-[(R)-2-oxo-5-phenyl-3-(tetrahydro-pyran-4-ylmethyl)-imidazolidin-1-yl]-piperidin-1-ylmethyl}-pyridin-2-yloxy)-benzoic acid

See EXAMPLE 52 for the preparation of (R)-4-phenyl-3-piperidin-4-yl-1-(tetrahydro-pyran-4-ylmethyl)-imidazolidin-2-one. COMPOUND 104 was isolated as a brown solid (50 mg, 64% over 2 steps). ¹H NMR (CDCl₃) δ 1.04-1.53 (m, 6H), 1.60-1.89 (m, 4H), 2.17 (s, 3H), 2.37 (s, 3H), 2.66-2.70 (m, 2H), 2.80-2.91 (m, 1H), 3.01-3.37 (m, 5H), 3.51-3.57 (m, 1H), 3.72 (t, 1H, J=9.3 Hz), 3.96 (br d, 2H, J=10.2 Hz), 4.05-4.21 (m, 2H), 4.69-4.62 (m, 1H), 6.72 (d, 1H, J=9.0 Hz), 7.01 (d, 1H, J=9.0 Hz), 7.29-7.34 (m, 5H), 7.83 (d, 1H, J=6.0 Hz), 7.93 (s, 1H), 8.29 (d, 1H, J=9.0 Hz); ¹³C NMR (CDCl₃) δ 17.3, 21.7, 23.3, 26.8, 28.7, 30.6, 31.5, 34.7, 50.0, 50.7, 52.6, 53.0, 54.5, 55.7, 57.1, 68.4, 110.0, 118.3, 122.2, 127.5, 129.4, 130.0, 130.1, 131.6, 134.2, 143.2, 145.4, 157.0, 158.1, 161.8, 163.6, 170.1, 176.3; ES-MS m/z 599 (M+H). Anal. Calcd. for C₃₅H₄₂N₄O₅1.8CH₂Cl₂.0.3CH₃OH.1.2C₂H₆O: C, 58.10; H, 6.67; N, 6.86. Found: C, 57.94; H, 6.40; N, 6.48.

### EXAMPLE 105

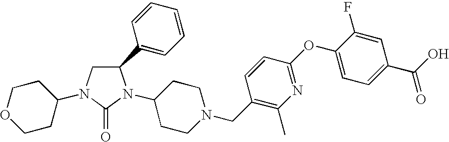

### COMPOUND 105: 3-Fluoro-4-(6-methyl-5-{4-[(R)-2-oxo-5-phenyl-3-(tetrahydro-pyran-4-yl)-imidazolidin-1-yl]-piperidin-1-ylmethyl}-pyridin-2-yloxy)-benzoic acid

3-Fluoro-4-(5-formyl-6-methyl-pyridin-2-yloxy)-benzonitrile was prepared using the same chemistry as 4-(5-formyl-6-methyl-pyridin-2-yloxy)-2-methyl-benzonitrile (see EXAMPLE 103) except that 4-bromo-2-fluorophenol was used in lieu of 4-bromo-3-methylphenol. COMPOUND 105 was isolated as a yellow foam (51% over 2 steps). ¹H NMR (CD₃OD) δ 1.35 (ddd, 1H, J=24.6, 12.3, 3.9 Hz), 1.47-1.51 (m, 1H), 1.65-1.86 (m, 5H), 1.97-2.14 (m, 3H), 2.36 (s, 3H), 2.74-2.78 (m, 1H), 2.90-2.93 (m, 1H), 3.16 (dd, 1H, J=8.7, 6.6 Hz), 3.43 (s, 2H), 3.47-3.59 (m, 3H), 3.80 (t, 1H, J=9.3 Hz), 3.92-4.03 (m, 3H), 4.77 (dd, 1H, J=9.3, 6.6 Hz), 6.72 (d, 1H, J=8.1 Hz), 7.18-7.24 (m, 1H), 7.32-7.42 (m, 5H), 7.63 (d, 1H, J=8.4 Hz), 7.83-7.89 (m, 2H); ES-MS m/z 611 (M+Na). Anal. Calcd. for C₃₃H₃₇N₄FO₅.0.6CH₂Cl₂: C, 63.09; H, 6.02; N, 8.76. Found: C, 63.28; H, 6.17; N, 8.47.

### EXAMPLE 106

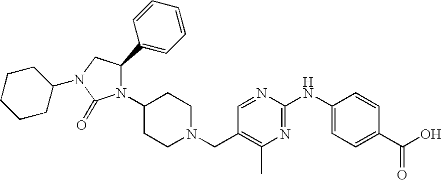

### COMPOUND 106: 4-{5-[4-((R)-3-Cyclohexyl-2-oxo-5-phenyl-imidazolidin-1-yl)-piperidin-1-ylmethyl]-4-methyl-pyrimidin-2-ylamino}-benzoic acid

COMPOUND 106 was isolated as a brown solid (0.078 g, 33% over 2 steps). ¹H NMR (CD₃OD) δ 1.13-1.18 (m, 1H), 1.33-1.56 (m, 5H), 1.63-1.84 (m, 7H), 2.09-2.24 (m, 1H), 2.33-2.42 (m, 2H), 2.49 (s, 3H), 3.00-3.04 (m, 1H), 3.15 (dd, 1H, J=9.0, 7.2 Hz), 3.54-3.63 (m, 1H), 3.69-3.77 (m, 3H), 3.79 (t, 1H, J=9.0 Hz), 4.74 (dd, 1H, J=9.0, 6.9 Hz), 7.35-7.42 (m, 5H), 7.84-7.88 (m, 2H), 7.96-8.00 (m, 2H), 8.27 (s, 1H); ¹³C NMR (CD₃OD) δ 20.40, 21.35, 25.53, 25.71, 25.80, 27.64, 28.99, 29.93, 30.29, 51.17, 52.07, 52.18, 52.52, 55.19, 56.79, 116.79, 118.06, 124.87, 127.03, 128.52, 129.12, 130.79, 142.61, 144.77, 159.67, 160.13, 168.87; ES-MS m/z 569 (M+H). Anal. Calcd. for C₃₃H₄₀N₆O₃.0.8CH₂Cl₂.1.3H₂O: C, 63.58; H, 6.99; N, 11.80. Found: C, 63.77; H, 7.06; N, 11.91.

### EXAMPLE 107

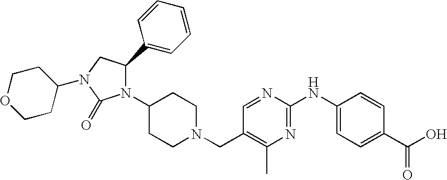

### COMPOUND 107: 4-(4-Methyl-5-5-{4-[(R)-2-oxo-5-phenyl-3-(tetrahydro-pyran-4-yl)-imidazolidin-1-yl]-piperidin-1-ylmethyl}-pyrimidin-2-ylamino)-benzoic acid

COMPOUND 107 was isolated as a red-orange powder (200 mg, 60% over 2 steps). ¹H NMR (CD₃OD) δ 1.64-1.91 (m, 7H), 2.27-2.39 (m, 1H), 2.52 (s, 3H), 2.78-2.96 (m, 2H), 3.17 (dd, 1H, J=8.7, 7.2 Hz), 3.33-3.54 (m, 2H), 3.50 (t, 2H, J=11.7 Hz), 3.65 (m, 1H), 3.81 (t, 1H, J=9.0 Hz), 3.90-4.03 (m, 3H), 4.06 (s, 2H), 4.76 (dd, 1H, J=9.3, 7.2 Hz), 7.34-7.42 (m, 5H), 7.86 (d, 2H, J=8.1 Hz), 7.97 (d, 2H, J=8.1 Hz), 8.40 (s, 1H); ¹³C NMR (CD₃OD) δ 21.6, 27.1, 27.8, 29.8, 30.2, 49.5, 50.4, 52.1, 52.2, 53.9, 54.8, 57.1, 67.2, 67.3, 114.5, 118.3, 124.6, 127.1, 128.7, 129.2, 130.9, 142.1, 144.7, 160.0, 160.6, 161.0, 169.2; ES-MS m/z 571 (M+H). Anal. Calcd. for C₃₂H₃₈N₆O₄.1.0CH₂Cl₂: C, 60.46; H, 6.15; N, 12.82. Found: C, 60.46; H, 6.47; N, 12.51.

### EXAMPLE 108

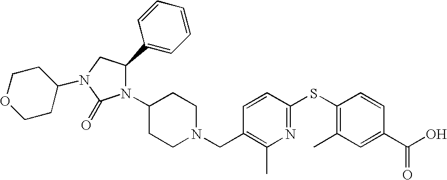

### COMPOUND 108: 3-Methyl-4-(6-methyl-5-{4-[(R)-2-oxo-5-phenyl-3-(tetrahydro-pyran-4-yl)-imidazolidin-1-yl]-piperidin-1-ylmethyl}-pyridin-2-ylsulfanyl)-benzoic acid

To a suspension of 4-bromo-2-methylaniline (5.40 g, 29.0 mmol) in aqueous (6 N, 14 mL) cooled at 0° C. was added a solution of NaNO₂ (2.27 g, 36.2 mmol) in water (5 mL) slowly. After addition the mixture was stirred at 0° C. for 30 min to give a clear solution. The solution was then added very slowly using a pipette to a solution of O-ethylxanthic acid potassium salt (5.81 g, 36.2 mmol) in water (10 mL) preheated at 40° C. (cautions, potential explosion hazard). After addition the mixture was stirred at 45° C. for 20 min, cooled to room temperature and extracted with Et₂O (3×50 mL). The combined extract was washed with aqueous NaOH (2 N, 40 mL) and water (2×30 mL), and dried over anhydrous Na₂SO₄. After filtration the solvent was removed to give a brown oil. The oil was dissolved in ethanol (30 mL) and heated to 70° C. KOH (7 g) was then added and the mixture was heated at reflux for 16 h. The mixture was then cooled to room temperature, washed with Et₂O (30 mL) and acidified with 6 N HCl to pH=3. Extraction with EtOAc (3×30 mL) was followed by drying over anhydrous Na₂SO₄. After filtration the solvent was removed, and the residue was stirred with 6-chloro-2-methylpyridine-3-carboxaldehyde (2.00 g, 12.8 mmol) and K₂CO₃ (2.00 g, 14.5 mmol) in DMF (20 mL) for 1 h. The mixture was concentrated and aqueous HCl (1 N, 15 mL) and water (20 mL) were added. Extraction with EtOAc (3×30 mL) was performed and the extracts were dried over anhydrous Na₂SO₄. After filtration the solvent was removed, and the residue was purified by flash chromatography on basic Al₂O₃ gel (EtOAc/hexanes, 1:4 in v/v) to afford 6-(4-bromo-2-methyl-phenylsulfanyl)-2-methyl-pyridine-3-carbaldehyde as a yellow oil (2.91 g, 70%). ¹H NMR (CDCl₃) δ 2.38 (s, 3H), 2.80 (s, 3H), 6.60 (d, 1H, J=8.1 Hz), 7.42 (dd, 1H, J=8.1, 1.8 Hz), 7.47 (d, 1H, J=8.1 Hz), 7.55 (d, 1H, J=1.8 Hz), 7.81 (d, 1H, J=8.1 Hz), 10.20 (s, 1H).

Under N₂, to a dry flask charged with 6-(4-bromo-2-methyl-phenylsulfanyl)-2-methyl-pyridine-3-carbaldehyde (2.75 g, 8.54 mmol), Zn(CN)₂ (0.587 g, 5.00 mmol), dppf (0.059 g, 0.107 mmol) and Pd₂(dba)₃ (0.039 g, 0.043 mmol) was added anhydrous DMF (40 mL). The mixture was stirred at 135° C. for 16 h and then cooled to room temperature. DMF was removed, water (30 mL) was added and the mixture was extracted with CH₂Cl₂ (3×30 mL). The combined extract was dried over anhydrous Na₂SO₄. After filtration the solvent was removed, and the residue was purified by flash chromatography on silica gel (EtOAc/hexanes, 1:2 in v/v) to afford 4-(5-formyl-6-methyl-pyridin-2-ylsulfanyl)-3-methyl-benzonitrile as a pale yellow solid (0.230 g, 10%). ¹H NMR (CDCl₃) δ 2.44 (s, 3H), 2.78 (s, 3H), 6.78 (d, 1H, J=8.4 Hz), 7.52 (dd, 1H, J=8.1, 1.2 Hz), 7.65 (d, 1H, J=1.2 Hz), 7.70 (d, 1H, J=8.1 Hz), 7.87 (d, 1H, J=8.4 Hz), 10.22 (s, 1H).

COMPOUND 108 was isolated as a brown solid (0.106 g, 44% over 2 steps). ¹H NMR (CD₃OD) δ 1.36-1.42 (m, 1H), 1.49-1.54 (m, 1H), 1.67-1.81 (m, 5H), 2.05-2.23 (m, 3H), 2.43 (s, 3H), 2.49 (s, 3H), 2.82-2.68 (m, 2H), 2.96-3.00 (m, 2H), 3.15 (dd, 1H, J=8.7, 6.6 Hz), 3.47-3.58 (m, 5H), 3.77-3.83 (m, 1H), 3.95-4.02 (m, 3H), 4.73 (dd, 1H, J=9.3, 6.6 Hz), 6.58 (d, 1H, J=8.1 Hz), 7.33-7.39 (m, 5H), 7.45 (d, 1H, J=8.1 Hz), 7.54 (d, 1H, J=7.8 Hz), 7.83-7.86 (m, 1H), 7.98 (s, 1H); ¹³C NMR (CD₃OD) δ 21.12, 21.97, 29.34, 30.87, 30.97, 31.27, 50.58, 53.00, 53.76, 54.16, 57.79, 59.07, 68.31, 68.39, 119.97, 127.75, 128.14, 129.30, 129.57, 130.15, 133.10, 134.82, 136.55, 138.17, 141.14, 142.92, 143.72, 159.84, 160.13, 161.96, 172.77; ES-MS m/z 601 (M+H). Anal. Calcd. for C₃₄H₄₀N₄O₄S.0.3NH₃.1.2H₂O: C, 65.08; H, 6.95; N, 9.60; S, 5.11. Found: C, 65.12; H, 6.76; N, 9.58; S, 5.07.

Examples 109 to 124 were prepared following the scheme illustrated below. RCHO is as defined in the table and X is as defined in the individual examples.

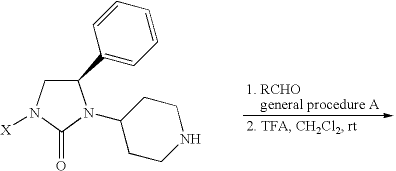

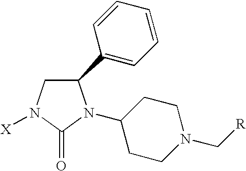

| Example | RCHO |
|---|---|
| 109 | [4-(5-formyl-6-methyl-pyridin-2-yloxy)-phenoxy]-acetic acid |
| | tert-butyl ester |
| 110 | 4-(5-formyl-6-methyl-pyridin-2-yloxy)-benzoic acid tert-butyl |
| | ester |
| 111 | [4-(5-formyl-6-methyl-pyridin-2-ylsulfanyl)-phenoxy]-acetic |
| | acid tert-butyl ester |
| 112 | [4-(5-formyl-6-methyl-pyridin-2-yloxy)-phenylsulfanyl]-acetic |
| | acid tert-butyl ester |
| 113 | 4-(5-formyl-6-methyl-pyridin-2-yloxy)-benzoic acid tert-butyl |
| | ester |
| 114 | 4-(5-formyl-6-methyl-pyridin-2-yloxy)-benzoic acid tert-butyl |
| | ester |
| 115 | [4-(5-formyl-pyridin-2-ylsulfanyl)-phenoxy]-acetic acid |
| | tert-butyl ester |
| 116 | [4-(5-formyl-pyridin-2-ylsulfanyl)-phenoxy]-acetic acid |
| | tert-butyl ester |
| 117 | 4-(6-ethyl-5-formyl-pyridin-2-yloxy)-benzoic acid tert-butyl |
| | ester |
| 118 | [4-(5-formyl-6-methyl-pyridin-2-ylsulfanyl)-phenoxy]-acetic |
| | acid tert-butyl ester |
| 119 | [4-(5-formyl-6-methyl-pyridin-2-ylsulfanyl)-phenoxy]-acetic |
| | acid tert-butyl ester |
| 120 | 4-(6-ethyl-5-formyl-pyridin-2-yloxy)-benzoic acid tert-butyl |
| | ester |
| 121 | [4-(5-formyl-6-methyl-pyridin-2-yloxy)-phenoxy]-acetic acid |
| | tert-butyl ester |
| 122 | [4-(5-formyl-pyridin-2-ylsulfanyl)-phenoxy]-acetic acid |
| | tert-butyl ester |
| 123 | [4-(5-formyl-6-methyl-pyridin-2-yloxy)-phenoxy]-acetic acid |
| | tert-butyl ester |
| 124 | 4-(5-formyl-6-methyl-pyridin-2-yloxy)-benzoic acid tert-butyl |
| | ester |

### EXAMPLE 109

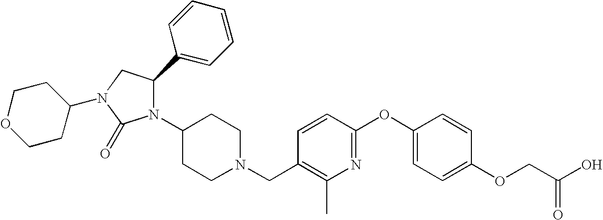

### COMPOUND 109: [4-(6-Methyl-5-{4-[(R)-2-oxo-5-phenyl-3-(tetrahydro-pyran-4-yl)-imidazolidin-1-yl]-piperidin-1-ylmethyl}-pyridin-2-yloxy)-phenoxy]-acetic acid

COMPOUND 109 was isolated as a white solid (0.455 g, 41% over 2 steps). ¹H NMR (CD₃OD) δ 1.57-1.82 (m, 7H), 2.21-2.27 (m, 1H), 2.44 (s, 3H), 2.56-2.68 (m, 2H), 3.11-3.20 (m, 2H), 3.23-3.27 (m, 1H), 3.46-3.55 (m, 2H), 3.60-3.68 (m, 1H), 3.81 (t, 1H, J=9.0 Hz), 3.89 (s, 2H), 3.95-4.03 (m, 3H), 4.44 (s, 2H), 4.76 (dd, 1H, J=9.0, 6.9 Hz), 6.58 (d, 1H, J=8.4 Hz), 6.94-7.03 (m, 4H), 7.35-7.42 (m, 5H), 7.67 (d, 1H, J=8.4 Hz); ES-MS m/z 601 (M+H). Anal. Calcd. for C₃₄H₄₀N₄O₆.0.8CH₂Cl₂.0.5TFA: C, 59.25; H, 5.85; N, 7.72. Found: C, 59.37; H, 5.88; N, 7.68.

### EXAMPLE 110

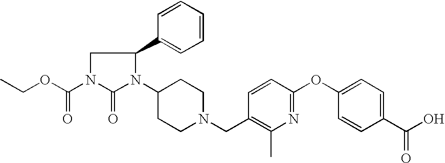

### COMPOUND 110: (R)-3-{1-[6-(4-Carboxy-phenoxy)-2-methyl-pyridin-3-ylmethyl]-piperidin-4-yl}-2-oxo-4-phenyl-imidazolidine-1-carboxylic acid ethyl ester

COMPOUND 110 was isolated as a white powder (62 mg, 94%). ¹H NMR (CDCl₃) δ 1.29 (t, 3H, J=7.2 Hz), 1.53 (br s, 2H), 1.84 (d, 1H, J=11.7 Hz), 2.31 (d, 1H, J=11.1 Hz), 2.42 (s, 3H), 2.59-2.70 (m, 2H), 3.19 (br s, 1H), 3.41 (br s, 1H), 3.63 (dd, 1H, J=10.5, 4.5 Hz), 3.94 (br s, 2H), 3.96-4.06 (m, 1H), 4.13 (t, 1H, J=10.2 Hz), 4.24 (q, 2H, J=7.2 Hz), 4.65 (dd, 1H, J=9.3, 4.8 Hz), 6.67 (d, 1H, J=8.1 Hz), 7.12 (d, 2H, J=8.4 Hz), 7.23-7.28 (m, 5H), 7.72 (d, 1H, J=8.1 Hz), 8.01 (d, 2H, J=8.4 Hz); ¹³C NMR (CDCl₃) δ 14.14, 21.95, 26.95, 28.16, 50.26, 51.73, 54.25, 56.90, 62.57, 109.15, 118.44, 119.84, 126.26, 127.41, 128.83, 129.16, 131.64, 140.67, 142.82, 151.70, 153.92, 157.09, 157.91, 161.79, 169.61; ES-MS m/z 518 (M+H). Anal. Calcd. for C₃₁H₃₄N₄O₆.1.1CH₂Cl₂: C, 59.13; H, 5.60; N, 8.59. Found: C, 59.42; H, 5.58; N, 8.50.

### EXAMPLE 111

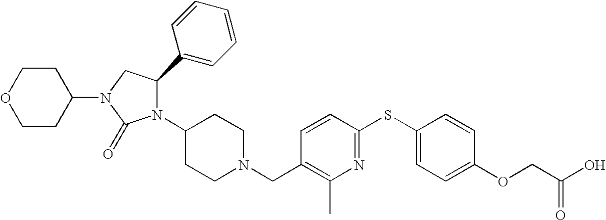

### COMPOUND 111: [4-(6-Methyl-5-{4-[(R)-2-oxo-5-phenyl-3-(tetrahydro-pyran-4-yl)-imidazolidin-1-yl]-piperidin-1-ylmethyl}-pyridin-2-ylsulfanyl)-phenoxy]-acetic acid

COMPOUND 111 was isolated as a white powder (94.4 mg, 90% over 2 steps). ¹H NMR (CD₃OD) δ 1.60-1.93 (m, 7H), 2.30-2.43 (m, 1H), 2.55 (s, 3H), 3.03-3.08 (m, 2H), 3.18 (t, 1H, J=8.1 Hz), 3.35-3.46 (m, 4H), 3.65 (m, 1H), 3.82 (t, 1H, J=9.0 Hz), 3.93-4.02 (m, 3H), 4.24 (s, 2H), 4.74 (s, 2H), 4.75 (t, 1H, J=8.1 Hz), 6.65 (d, 1H, J=8.4 Hz), 7.08 (d, 2H, J=8.7 Hz), 7.28-7.43 (m, 514), 7.51-7.56 (m, 3H); ¹³C NMR (CD₃OD) δ 21.5, 26.8, 27.6, 30.0, 30.5, 48.8, 49.7, 50.1, 52.3, 52.5, 56.7, 57.5, 65.4, 67.4, 67.5, 116.7, 118.4, 120.0, 121.3, 12.4, 129.0, 129.5, 137.8, 141.4, 142.1, 159.2, 160.3, 160.8, 165.0, 171.8; ES-MS m/z 617 (M+H). Anal. Calcd. for C₃₄H₄₀N₄O₅S.1.3CH₂Cl₂: C, 58.31; H, 5.90; N, 7.70. Found: C, 58.53; H, 5.78; N, 7.55.

### EXAMPLE 112

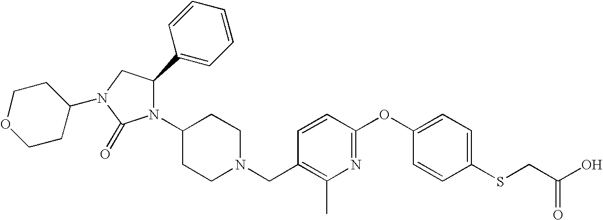

### COMPOUND 112: [4-(6-Methyl-5-{4-[(R)-2-oxo-5-phenyl-3-(tetrahydro-pyran-4-yl)-imidazolidin-1-yl]-piperidine-1-ylmethyl}-pyridin-2-yloxy)-phenylsulfanyl]-acetic acid

COMPOUND 112 was isolated as a white solid (32 mg, 36% over 2 steps). ¹H NMR (CD₃OD) δ 1.60-1.90 (m, 7H), 2.35 (dq, 1H, J=12.0, 3.6 Hz), 2.47 (s, 3H), 3.00 (m, 2H), 3.19 (t, 1H, J=8.1 Hz), 3.35-3.70 (m, 5H), 3.72 (s, 2H), 3.83 (t, 1H, J=9.0 Hz), 4.00 (m, 3H), 4.22 (s, 2H), 4.76 (t, 1H, J=7.5 Hz), 6.80 (d, 1H, J=8.4 Hz), 7.10 (d, 2H, J=8.7 Hz), 7.35-7.45 (m, 5H), 7.50 (d, 2H, J=8.7 Hz), 7.78 (d, 1H, J=8.4 Hz); ES-MS m/z 617 (M+H).

### EXAMPLE 113

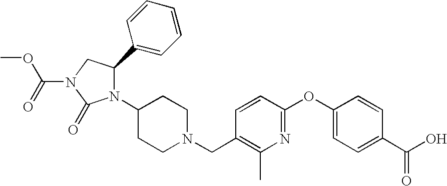

### COMPOUND 113: (R)-3-{1-[6-(4-Carboxy-phenoxy)-2-methyl-pyridin-3-ylmethyl]-piperidin-4-yl}-2-oxo-4-phenyl-imidazolidine-1-carboxylic acid methyl ester

COMPOUND 113 was isolated as a pale brown powder (31.4 mg, 30% over 2 steps). ¹H NMR (CD₃OD) δ 1.85 (m, 3H), 2.30-2.43 (m, 1H), 2.46 (s, 3H), 2.81 (t, 2H, J=11.7 Hz), 3.28-3.38 (m, 2H), 3.63-3.70 (m, 1H), 3.66 (dd, 1H, J=10.5, 5.4 Hz), 3.84 (s, 3H), 4.07 (s, 2H), 4.27 (t, 1H, J=10.2 Hz), 4.88 (dd, 1H, J=9.6, 5.4 Hz), 6.86 (d, 1H, J=8.4 Hz), 7.18 (d, 2H, J=7.2 Hz), 7.38-7.48 (m, 5H), 7.81 (d, 1H, J=8.4 Hz), 8.08 (d, 2H, J=7.2 Hz); ¹³C NMR (CD₃OD) δ 21.1, 27.0, 27.5, 50.5, 51.0, 52.1, 52.3, 53.1, 56.1, 57.0, 109.7, 120.3, 121.4, 127.2, 129.2, 129.5, 131.8, 140.9, 144.1, 153.0, 155.1, 158.3, 162.9, 163.5; ES-MS m/z 545 (M+H). Anal. Calcd. for C₃₀H₃₂N₄O₆.2.0CH₂Cl₂: C, 53.80; H, 5.08; N, 7.84. Found: C, 53.66; H, 4.93; N, 7.61.

### EXAMPLE 114

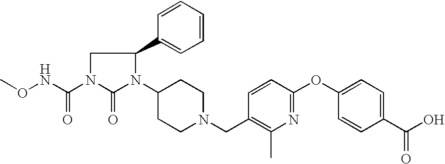

### COMPOUND 114: 4-{5-[4-((R)-3-Methoxycarbamoyl-2-oxo-5-phenyl-imidazolidin-1-yl)-piperidin-1-ylmethyl]-6-methyl-pyridin-2-yloxy}-benzoic acid

COMPOUND 114 was isolated as a white powder (91 mg, 84% over 2 steps). ¹H NMR (CD₃OD) δ 1.58-1.81 (m, 3H), 2.17-2.31 (m, 1H), 2.45 (s, 3H), 2.47 (m, 2H), 3.06 (d, 1H, J=11.1 Hz), 3.17 (d, 1H, J=11.1 Hz), 3.55-3.62 (m, 2H), 3.78 (s, 3H), 3.79 (m, 2H), 4.19 (t, 1H, J=10.2 Hz), 4.89 (m, 1H), 6.80 (d, 1H, J=8.1 Hz), 7.15 (d, 2H, J=6.9 Hz), 7.37-7.46 (m, 5H), 7.74 (d, 1H, J=8.1 Hz), 8.06 (br s, 2H); ¹³C NMR (CD₃OD) δ 22.49, 28.91, 29.67, 50.59, 52.93, 53.67, 53.81, 55.37, 57.74, 58.66, 65.57, 110.84, 121.41, 124.53, 128.49, 130.48, 130.84, 133.20, 142.58, 145.00, 156.17, 157.37, 159.29, 159.58, 163.89; ES-MS m/z 560 (M+H). Anal. Calcd. for C₃₀H₃₃N₅O₆.1.0CH₂Cl₂: C, 57.77; H, 5.47; N, 10.86. Found: C, 57.82; H, 5.46; N, 10.65.

### EXAMPLE 115

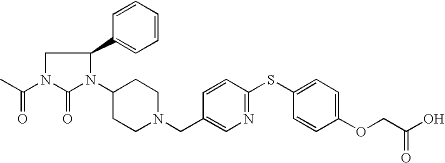

### COMPOUND 115: (4-{5-[4-((R)-3-Acetyl-2-oxo-5-phenyl-imidazolidin-1-yl)-piperidin-1-ylmethyl]-pyridin-2-ylsulfanyl}-phenoxy)-acetic acid

COMPOUND 115 was isolated as a white solid (57.7 mg, 68% over 2 steps). ¹H NMR (CD₃OD) δ 1.83-1.90 (m, 3H), 2.32-2.44 (m, 1H), 2.52 (s, 3H), 2.80-2.88 (m, 2H), 3.28-3.38 (m, 2H), 3.59 (dd, 1H, J=11.4, 5.4 Hz), 3.17 (d, 1H, J=11.1 Hz), 3.70 (m, 1H), 4.07 (s, 2H), 4.18 (t, 1H, J=10.5 Hz), 4.55 (s, 2H), 4.82 (dd, 1H, J=9.6, 5.4 Hz), 6.82 (d, 1H, J=8.4 Hz), 7.02 (d, 2H, J=8.4 Hz), 7.37-7.43 (m, 5H), 7.48 (d, 2H, J=8.4 Hz), 7.60 (d, 1H, J=8.4 Hz), 8.36 (s, 1H); ¹³C NMR (CD₃OD) δ 24.10, 27.91, 28.56, 50.95, 51.91, 52.95, 53.23, 56.92, 58.37, 67.98, 117.86, 121.78, 123.89, 128.47, 130.46, 130.88, 138.91, 141.43, 142.74, 152.78, 156.38, 161.87, 166.47, 172.85, 175.06; ES-MS m/z 561 (M+H); Anal. Calcd. for C₃₀H₃₂N₄O₅S.0.8CH₂Cl₂: C, 58.85; H, 5.39; N, 8.91; Found: C, 59.11; H, 5.40; N, 8.82.

### EXAMPLE 116

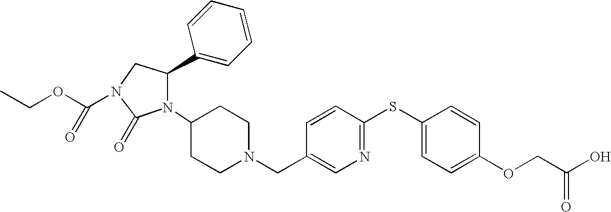

### COMPOUND 116: (R)-3-{1-[6-(4-Carboxymethoxy-phenylsulfanyl)-pyridin-3-ylmethyl]-piperidin-4-yl}-2-oxo-4-phenyl-imidazolidine-1-carboxylic acid ethyl ester

COMPOUND 116 was isolated as a white powder (64.8 mg, 88% over 2 steps). ¹H NMR (CD₃OD) δ 1.28-1.35 (m, 3H), 1.50-1.76 (m, 3H), 2.14-2.26 (m, 3H), 2.93 (d, 1H, J=11.4 Hz), 3.02 (d, 1H, J=10.2 Hz), 3.50-3.60 (m, 2H), 3.63 (s, 2H), 4.19-4.29 (m, 3H), 4.49 (s, 2H), 4.84 (dd, 1H, J=9.3, 5.1 Hz), 6.76 (t, 1H, J=7.2 Hz), 7.05 (m, 2H), 7.40-7.55 (m, 8H), 8.26 (s, 1H); ¹³C NMR (CDCl₃) δ 14.7, 23.0, 25.5, 28.0, 30.1, 49.6, 50.7, 53.8, 55.1, 63.0, 65.8, 116.4, 120.8, 121.2, 127.0, 129.6, 129.8, 137.8, 139.6, 140.8, 151.5, 151.9, 154.4, 159.8, 165.3, 171.8; ES-MS m/z 591 (M+H).

### EXAMPLE 117

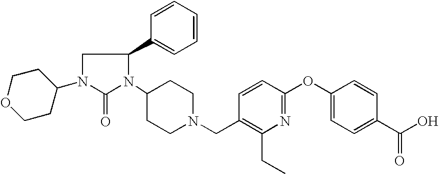

### COMPOUND 117: 4-(6-Ethyl-5-{4-[(R)-2-oxo-5-phenyl-3-(tetrahydro-pyran-4-yl)-imidazolidin-1-yl]-piperidin-1-ylmethyl}-pyridin-2-yloxy)-benzoic acid

COMPOUND 117 was isolated as a white powder (71 mg, 59% over 2 steps). ¹H NMR (CDCl₃) δ 1.11 (t, 3H, J=7.5 Hz), 1.25-1.42 (m, 2H), 1.66 (m, 5H), 2.14-2.28 (m, 3H), 1.11 (q, 2H, J=7.5 Hz), 2.88 (br s, 1H), 3.08 (m, 2H), 3.48-3.81 (m, 5H), 4.01 (m, 3H), 4.59 (t, 1H, J=7.5 Hz), 6.62 (d, 1H, J=9 Hz), 7.05-7.26 (m, 5H), 7.14 (d, 2H, J=9 Hz), 7.65 (br s, 1H), 8.02 (br s, 2H); ¹³C NMR (CDCl₃) δ 13.74, 28.01, 30.20, 30.52,48.74, 49.14, 51.52, 52.96, 56.33, 57.64, 67.53, 67.62, 109.12, 120.24, 122.03, 127.09, 128.39, 128.72, 129.30, 131.97, 142.66, 142.88, 158.56, 160.33, 161.79, 162.07, 170.32; ES-MS m/z 585 (M+H). Anal. Calcd. for C₃₃H₄₀N₄O_{5.}0.5CH₂Cl₂: C, 66.07; H, 6.59; N, 8.93. Found: C, 66.02; H, 6.65; N, 8.91.

### EXAMPLE 118

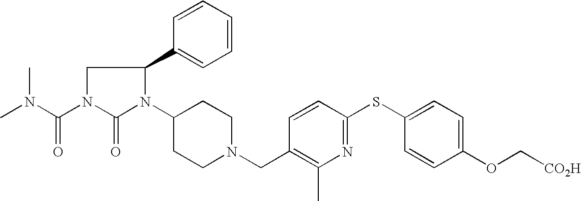

### COMPOUND 118: (4-{5-[4-((R)-3-Dimethylcarbamoyl-2-oxo-5-phenyl-imidazolidin-1-yl)-piperidin-1-ylmethyl]-6-methyl-pyridin-2-ylsulfanyl}-phenoxy)-acetic acid

COMPOUND 118 was isolated as a white foam (111 mg, 75% over 2 steps). ¹H NMR (CDCl₃) δ 1.56-1.69 (m, 2H), 1.84 (d, 1H, J=12.6 Hz), 2.30-2.45 (m, 1H), 2.48 (s, 3H), 2.56-2.71 (m, 2H), 3.03 (s, 6H), 3.32 (d, 1H, J=10.8 Hz), 3.46 (dd, 2H, J=9.8, 3.8 Hz), 3.91-4.05 (m, 3H), 4.12 (t, 1H, J=9.5 Hz), 4.48 (s, 2H), 4.65 (dd, 1H, J=8.6, 4.4 Hz), 6.40 (d, 1H, J=8.4 Hz), 6.97 (d, 2H, J=7.8 Hz), 7.26-7.31 (m, 5H), 7.43-7.48 (m, 3H); ¹³C NMR (CDCl₃) 22.73, 26.68, 27.80, 38.27, 49.78, 51.54, 51.97, 55.43, 56.33, 66.39, 116.54, 118.35, 119.89, 121.01, 126.80, 129.20, 129.60, 137.84, 140.48, 141.08, 155.71, 156.32, 157.86, 159.90, 164.71, 172.68; ES-MS m/z 604 (M+H); Anal. Calcd. for C₃₂H₃₇N₅O₅S.2.4CH₂Cl₂: C, 51.16; H, 5.22; N, 8.67. Found: C, 50.91; H, 5.10; N, 8.66.

### EXAMPLE 119

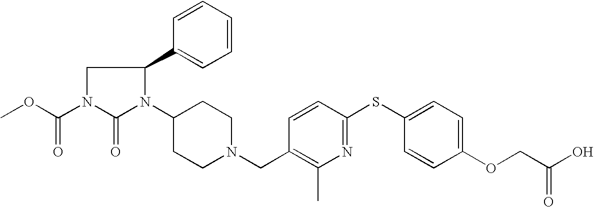

### COMPOUND 119: (R)-3-{1-[6-(4-Carboxymethoxy-phenylsulfanyl)-2-methyl-pyridin-3-ylmethyl]-piperidin-4-yl}-2-oxo-4-phenyl-imidazolidine-1-carboxylic acid methyl ester

COMPOUND 119 was isolated as a yellow powder (53.2 mg, 64% over 2 steps). ¹H NMR (CD₃OD) δ 1.97 (br s, 3H), 2.42-2.51 (m, 1H), 2.59 (s, 3H), 3.11 (t, 2H, J=11.1 Hz), 3.51 (t, 2H, J=11.1 Hz), 3.65-3.75 (m, 2H), 3.85 (s, 3H), 4.26-4.34 (m, 1H), 4.30 (s, 2H), 4.80 (s, 2H), 4.89 (dd, 1H, J=9.6, 5.7 Hz), 6.70 (d, 1H, J=8.1 Hz), 7.12 (d, 2H, J=8.7 Hz), 7.44-7.52 (m, 5H), 7.57 (d, 2H, J=8.7 Hz), 7.60 (d, 1H, J=8.1 Hz); ES-MS m/z 591 (M+H). Anal. Calcd. for C₃₁H₃₄N₄O₆S.1.5CH₂Cl₂: C, 54.36; H, 5.19; N, 7.80. Found: C, 54.53; H, 4.99; N, 7.52.

### EXAMPLE 120

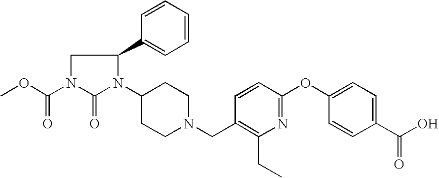

### COMPOUND 120: (R)-3-{1-[6-(4-Carboxy-phenoxy)-2-ethyl-pyridin-3-ylmethyl]-piperidin-4-yl}-2-oxo-4-phenyl-imidazolidine-1-carboxylic acid methyl ester

COMPOUND 120 was isolated as a white powder (74.4 mg, 72% over 2 steps). ¹H NMR (CDCl₃) δ 1.09 (t, 3H, J=7.5 Hz), 1.93 (br s, 1H), 2.45 (br s, 1H), 2.65-2.88 (m, 2H), 2.67 (q, 2H, J=7.5 Hz), 3.36 (m, 1H), 3.48 (s, 2H), 3.57-3.68 (m, 2H), 3.82 (s, 3H), 4.15 (br s, 4H), 4.69 (br s, 1H), 6.74 (d, 1H, J=8.1 Hz), 7.15 (d, 2H, J=8.1 Hz), 7.25-7.33 (m, 5H), 7.80 (d, 1H, J=8.1 Hz), 8.03 (d, 2H, J=8.1 Hz); ¹³C NMR (CD₃OD) δ 14.02, 27.89, 28.38, 28.86, 51.78, 51.88, 53.31, 53.55, 54.33, 55.31, 57.48, 111.05, 120.19, 122.16, 128.55, 128.82, 130.60, 130.90, 133.00, 142.18, 145.79, 154.12, 156.23, 159.73, 164.16, 164.70, 169.79; ES-MS m/z 559 (M+H). Anal. Calcd. for C₃₁H₃₄N₄O₆.1.7CH₂Cl₂: C, 55.87; H, 5.36; N, 7.97. Found: C, 56.09; H, 5.15; N, 7.93.

### EXAMPLE 121

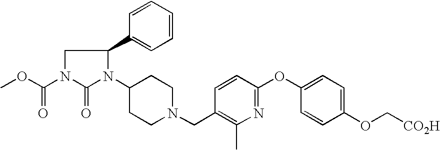

### COMPOUND 121: (R)-3-{1-[6-(4-Carboxymethoxy-phenoxy)-2-methyl-pyridin-3-ylmethyl]-piperidin-4-yl}-2-oxo-4-phenyl-imidazolidine-1-carboxylic acid methyl ester

COMPOUND 121 was isolated as a white powder (29 mg, 32% over 2 steps). ¹H NMR (CDCl₃) δ 1.73 (m, 3H), 2.20-2.55 (m, 4H), 2.33 (s, 3H), 3.00-3.30 (m, 2H), 3.51-3.80 (m, 4H), 3.77 (s, 3H), 4.10 (br s, 1H), 4.42 (br s, 1H), 4.63 (br s, 1H), 6.39 (br s, 1H), 6.89 (br s, 4H), 7.31 (br s, 5H), 7.55 (s, 1H); ¹³C NMR (CDCl₃) δ 22.47, 27.21, 28.27, 50.76, 52.22, 53.86, 55.03, 57.10, 67.55, 107.85, 116.09, 122.54, 126.84, 129.39, 129.71, 141.14, 143.11, 147.96, 152.72, 154.17, 155.72, 157.39, 163.81; ES-MS m/z 575 (M+H); Anal. Calcd. for C₃₁H₃₄N₄O₇.2.0CH₂Cl₂: C, 53.24; H, 5.14; N, 7.53. Found: C, 53.26; H, 5.13; N, 7.62.

### EXAMPLE 122

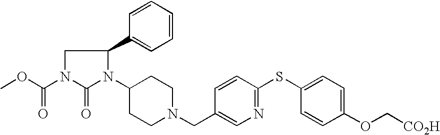

### COMPOUND 122: (R)-3-{1-[6-(4-Carboxymethoxy-phenylsulfanyl)-pyridin-3-ylmethyl]-piperidin-4-yl}-2-oxo-4-phenyl-imidazolidine-1-carboxylic acid methyl ester

COMPOUND 122 was isolated as a white foam (69.4 mg, 82% over 2 steps). ¹H NMR (CDCl₃) δ 1.50 (br s, 2H), 1.82 (d, 1H, J=11.1 Hz), 2.35-2.42 (m, 1H), 2.66-2.73 (m, 2H), 3.25-3.31 (m, 1H), 3.37-3.40 (m, 1H), 3.66 (dd, 1H, J=10.5, 4.8 Hz), 3.83 (s, 3H), 3.85-4.02 (m, 2H), 4.14 (t, 1H, J=10.2 Hz), 4.48 (s, 2H), 4.66 (dd, 1H, J=9.5, 5.0 Hz), 6.62 (d, 1H, J=8.4 Hz), 6.97 (d, 2H, J=8.4 Hz), 7.26-7.36 (m, 5H), 7.43 (d, H, J=8.4 Hz), 7.51 (d, 1H, J=7.2 Hz), 8.25 (s, 1H); ¹³C NMR (CDCl₃) δ 27.45, 28.65, 51.13, 51.93, 52.24, 52.85, 55.03, 56.08, 58.05, 67.73, 117.71, 121.82, 123.14, 128.06, 130.66, 130.95, 139.06, 140.73, 142.15, 152.50, 153.66, 155.27, 161.19, 166.52, 174.14; ES-MS m/z 577 (M+H); Anal. Calcd. for C₃₀H₃₂N₄O₆S.1.6CH₂Cl₂: C, 53.27; H, 4.98; N, 7.86. Found: C, 53.27; H, 5.09; N, 7.89.

### EXAMPLE 123

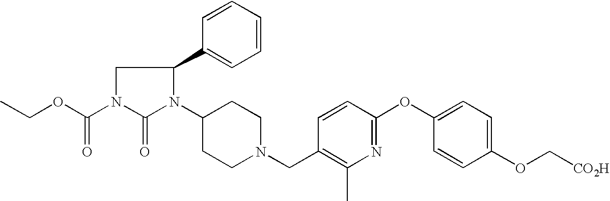

### COMPOUND 123: (R)-3-{1-[6-(4-Carboxymethoxy-phenoxy)-2-methyl-pyridin-3-ylmethyl]-piperidin-4-yl}-2-oxo-4-phenyl-imidazolidine-1-carboxylic acid ethyl ester

COMPOUND 123 was isolated as a yellow foam (106 mg, 69% over 2 steps). ¹H NMR (CDCl₃) δ 1.29 (t, 3H, J=7.1 Hz), 1.52-1.60 (m, 2H), 1.79 (d, 1H, J=11.1 Hz), 2.24-2.36 (m, 1H), 2.38 (s, 3H), 2.48-2.65 (m, 2H), 3.15 (d, 1H, J=10.5 Hz), 3.33 (d, 1H, J=9.9 Hz), 3.60 (dd, 1H, J=10.8, 4.5 Hz), 3.83-3.94 (m, 1H), 3.86 (s, 2H), 4.11 (t, 1H, J=9.9 Hz), 4.24 (q, 2H, J=7.1 Hz), 4.30 (s, 2H), 4.65 (dd, 1H, J=9.3, 4.5 Hz), 6.41 (d, 1H, J=8.4 Hz), 6.86 (d, 2H, J=8.9 Hz), 6.95 (d, 2H, J=8.9 Hz), 7.26-7.36 (m, 5H), 7.61 (d, 1H, J=8.4 Hz), 8.80 (br s, 1H); ¹³C NMR (CDCl₃) δ 14.71, 22.58, 27.00, 28.23, 50.41, 50.82, 51.65, 51.98, 54.67, 56.63, 62.94, 67.13, 107.94, 115.95, 120.12, 122.59, 126.79, 129.30, 129.68, 141.41, 143.35, 147.82, 151.99, 154,22, 155.85, 157.49, 163.99, 173.46; ES-MS m/z 589 (M+H). Anal. Calcd. For C₃₂H₃₆N₄O₇.1.0CH₂Cl₂: C, 58.84; H, 5.69; N, 8.32. Found: C, 58.99; H, 5.71; N, 8.22.

### EXAMPLE 124

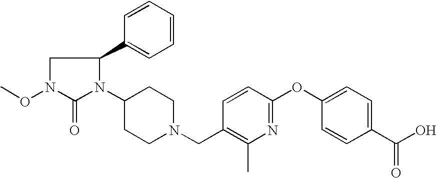

### COMPOUND 124: (R)-4-{5-[4-(3-Methoxy-2-oxo-5-phenyl-imidazolidin-1-yl)-piperidin-1ylmethoxy]-6-methyl-pyridin-2-yloxy}-benzoic acid

COMPOUND 124 was isolated as a white solid (72.2 mg, 53% over 2 steps). ¹H NMR (CD₃OD) δ 1.85-1.97 (m, 3H), 2.34-2.50 (m, 1H), 2.43 (s, 3H), 2.86 (t, 2H, J=11.7 Hz), 3.24 (t, 1H, J=7.8 Hz), 3.35-3.52 (m, 3H), 3.76 (s, 3H), 3.90 (t, 1H, J=7.8 Hz), 4.11 (s, 2H), 4.67 (t, 1H, J=7.8 Hz), 6.86 (d, 1H, J=8.4 Hz), 7.17 (d, 2H, J=8.4 Hz), 7.35-7.44 (m, 5H), 7.79 (d, 1H, J=8.4 Hz), (d, 2H, J=8.4 Hz); ¹³C NMR (CD₃OD) δ 22.52, 28.25, 28.35, 52.06, 53.39, 53.45, 56.61, 57.97, 64.16, 111.16, 121.72, 121.81, 129.07, 129.41, 130.48, 130.67, 133.10, 140.76, 145.57, 159.73, 159.77, 164.36, 164.64; ES-MS m/z 517 (M+H).

Examples 125 to 142 were prepared following the scheme illustrated below. RNH₂ is as defined in the table and X and Y are as defined in the individual examples.

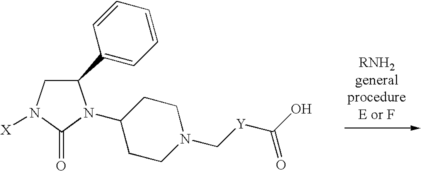

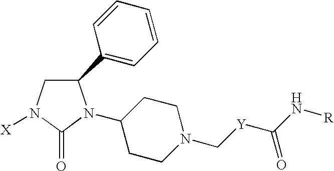

| Example | RNH₂ |
|---|---|
| 125 | methoxylamine hydrochloride |
| 126 | Isopropylamine |
| 127 | 1-aminopropane |
| 128 | Isopropylamine |
| 129 | Cyclopropylamine |
| 130 | methylamine hydrochloride |
| 131 | Cyclopropylamine |
| 132 | methylamine hydrochloride |
| 133 | methoxylamine hydrochloride |
| 134 | Cyclopropylamine |
| 135 | Cyclopropylamine |
| 136 | Cyclopropylamine |
| 137 | methoxylamine hydrochloride |
| 138 | Isopropylamine |
| 139 | methylamine hydrochloride |
| 140 | methylamine hydrochloride |
| 141 | methylamine hydrochloride |
| 142 | Cyclopropylamine |

### EXAMPLE 125

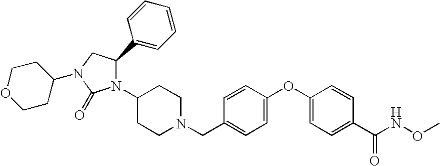

### COMPOUND 125: N-Methoxy-4-(4-{4-[(R)-2-oxo-5-phenyl-3-(tetrahydro-pyran-4-yl)-imidazolidin-1-yl]-piperidin-1-ylmethyl}-phenoxy)-benzamide

Following general procedure E: 4-(4-{4-[(R)-2-oxo-5-phenyl-3-(tetrahydro-pyran-4-yl)-imidazolidin-1-yl]-piperidin-1-ylmethyl}-phenoxy)-benzoic acid (COMPOUND 60) afforded COMPOUND 125 as a white solid (35 mg, 48%). ¹H NMR (CDCl₃) δ 1.22 (m, 1H), 1.41 (br m, 1H), 1.65 (br m, 5H), 1.83-2.04 (m, 3H), 2.70 (br d, 1H), 2.88 (br d, 1H), 3.05 (m, 1H), 3.40 (s, 2H), 3.47 (m, 2H), 3.65 (m, 2H), 3.88 (s, 3H), 4.00 (m, 3H), 4.60 (m, 1H), 6.95 (m, 4H), 7.22 (d, 2H, J=8.4 Hz), 7.33 (br s, 5H), 7.71 (d, 2H, J=8.4 Hz), 8.75 (br m, 1H); ES-MS m/z 585 (M+H).

### EXAMPLE 126

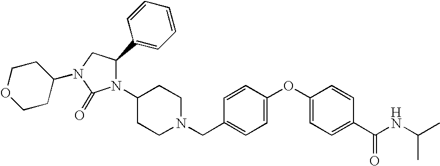

### COMPOUND 126: N-Isopropyl-4-(4-{4-[(R)-2-oxo-5-phenyl-3-(tetrahydro-pyran-4-yl)-imidazolidin-1-yl]-piperidin-1-ylmethyl}-phenoxy)-benzamide

Following general procedure F: 4-(4-{4-[(R)-2-oxo-5-phenyl-3-(tetrahydro-pyran-4-yl)-imidazolidin-1-yl]-piperidin-1-ylmethyl}-phenoxy)-benzoic acid (COMPOUND 60) afforded COMPOUND 126 as a white solid (40 mg, 51%). ¹H NMR (CDCl₃) δ 1.25 (m, 1H), 1.26 (d, 6H, J=6.9 Hz), 1.43 (d, 1H, J=10.8 Hz), 1.65 (m, 5H), 1.90 (m, 3H), 2.67 (d, 1H, J=10.8 Hz), 2.83 (d, 1H, J=10.8 Hz), 3.05 (m, 1H), 3.39 (s, 2H), 3.47 (m, 2H), 3.63 (t, 1H, J=9.0 Hz), 3.64 (m, 1H), 4.00 (m, 3H), 4.25 (sept, 1H, J=7.5 Hz), 4.60 (m, 1H), 5.83 (d, 1H, J=6.3 Hz), 6.95 (m, 4H), 7.22 (d, 2H, J=9.0 Hz), 7.33 (br s, 5H), 7.71 (d, 1H, J=7.8 Hz); ES-MS m/z 597 (M+H).

### EXAMPLE 127

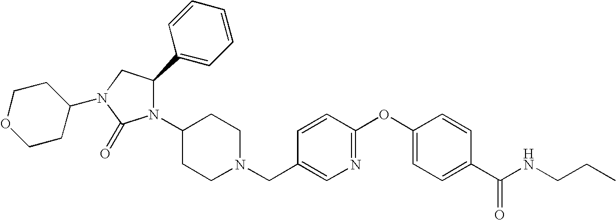

### COMPOUND 127: 4-(5-{4-[(R)-2-Oxo-5-phenyl-3-(tetrahydro-pyran-4-yl)-imidazolidin-1-yl]-piperidin-1-ylmethyl}-pyridin-2-yloxy)-N-propyl-benzamide

Following general procedure F: 4-(5-{4-[(R)-2-oxo-5-phenyl-3-(tetrahydro-pyran-4-yl)-imidazolidin-1-yl]-piperidin-1-ylmethyl}-pyridin-2-yloxy)-benzoic acid (COMPOUND 51) afforded COMPOUND 127 as a light beige solid (41 mg, 62%). ¹H NMR (CDCl₃) δ 0.99 (t, 3H, J=7.2 Hz), 1.22 (m, 1H), 1.41 (br m, 1H), 1.63 (br m, 7H), 1.86-2.06 (m, 3H), 2.67 (br d, 1H), 2.85 (br d, 1H), 3.06 (m, 1H), 3.37 (s, 2H), 3.38-3.49 (m, 4H), 3.65 (m, 2H), 4.00 (m, 3H), 4.59 (m, 1H), 6.07 (br m, 1H), 6.87 (d, 1H, J=8.1 Hz), 7.15 (d, 2H, J=8.1 Hz), 7.33 (br s, 5H), 7.62 (d, 1H, J=7.8 Hz), 7.78 (d, 2H, J=8.1 Hz), 8.00 (s, 1H); ES-MS m/z 598 (M+H).

### EXAMPLE 128

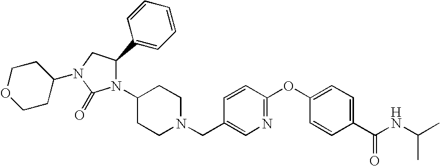

### COMPOUND 128: N-Isopropyl-4-(5-{4-[(R)-2-oxo-5-phenyl-3-(tetrahydro-pyran-4-yl)-imidazolidin-1-yl]-piperidin-1-ylmethyl}-pyridin-2-yloxy)-benzamide

Following general procedure F: 4-(5-{4-[(R)-2-oxo-5-phenyl-3-(tetrahydro-pyran-4-yl)-imidazolidin-1-yl]-piperidin-1-ylmethyl}-pyridin-2-yloxy)-benzoic acid (COMPOUND 51) afforded COMPOUND 128 as a light beige solid (41 mg, 61%). ¹H NMR (CDCl₃) δ 1.20 (m, 1H), 1.26 (d, 6H, J=6.3 Hz), 1.41 (br m, 1H), 1.65 (m, 5H), 1.83-2.02 (m, 3H), 2.65 (br d, 1H), 2.85 (br d, 1H), 3.06 (m, 1H), 3.36 (s, 2H), 3.46 (m, 2H), 3.65 (m, 2H), 3.98 (m, 3H), 4.27 (m, 1H), 4.58 (m, 1H), 5.85 (br d, 1H, J=7.2 Hz), 6.87 (d, 1H, J=8.1 Hz), 7.15 (d, 2H, J=8.1 Hz), 7.33 (br s, 5H), 7.61 (d, 1H, J=7.8 Hz), 7.77 (d, 2H, J=8.1 Hz), 7.99 (s, 1H); ES-MS m/z 598 (M+H).

### EXAMPLE 129

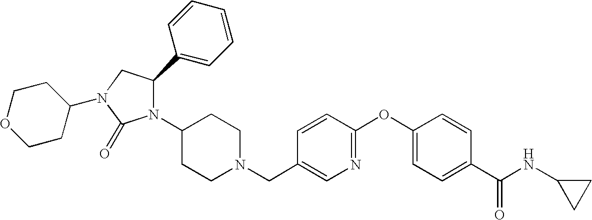

### COMPOUND 129: N-Cyclopropyl-4-(5-{4-[(R)-2-oxo-5-phenyl-3-(tetrahydro-pyran-4-yl)-imidazolidin-1-yl]-piperidin-1-ylmethyl}-pyridin-2-yloxy)-benzamide

Following general procedure F: 4-(5-{4-[(R)-2-oxo-5-phenyl-3-(tetrahydro-pyran-4-yl)-imidazolidin-1-yl]-piperidin-1-ylmethyl}-pyridin-2-yloxy)-benzoic acid (COMPOUND 51) gave COMPOUND 129 as a white solid (30 mg, 46%). ¹H NMR (CDCl₃) δ 0.61 (m, 2H), 0.87 (m, 2H), 1.20 (m, 1H), 1.41 (br m, 1H), 1.65 (br m, 5H), 1.83-2.02 (m, 3H), 2.65 (br d, 1H), 2.88 (m, 2H), 3.06 (m, 1H), 3.36 (s, 2H), 3.46 (m, 2H), 3.65 (m, 2H), 4.00 (m, 3H), 4.58 (m, 1H), 6.18 (br m, 1H), 6.86 (d, 1H, J=8.1 Hz), 7.15 (d, 2H, J=8.1 Hz), 7.33 (br s, 5H), 7.61 (d, 1H, J=7.8 Hz), 7.75 (d, 2H, J=8.1 Hz), 7.99 (s, 1H); ES-MS m/z 596 (M+H).

### EXAMPLE 130

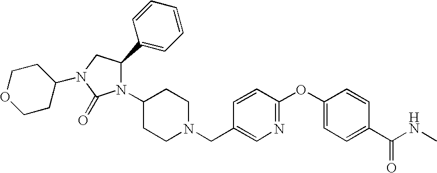

### COMPOUND 130: N-Methyl-4-(5-{4-[(R)-2-oxo-5-phenyl-3-(tetrahydro-pyran-4-yl)-imidazolidin-1-yl]-piperidin-1-ylmethyl}-pyridin-2-yloxy)-benzamide

Following general procedure E: 4-(5-{4-[(R)-2-oxo-5-phenyl-3-(tetrahydro-pyran-4-yl)-imidazolidin-1-yl]-piperidin-1-ylmethyl}-pyridin-2-yloxy)-benzoic acid (COMPOUND 51) afforded COMPOUND 130 as a white solid (39 mg, 77%). ¹H NMR (CDCl₃) δ 1.20 (m, 1H), 1.41 (br m, 1H), 1.65 (m, 5H), 1.83-2.02 (m, 3H), 2.67 (br d, 1H), 2.85 (br d, 1H), 3.01 (d, 3H, J=5.1 Hz), 3.06 (m, 1H), 3.36 (s, 2H), 3.46 (m, 2H), 3.65 (m, 2H), 4.00 (m, 3H), 4.59 (m, 1H), 6.09 (br m, 1H), 6.87 (d, 1H, J=8.1 Hz), 7.15 (d, 2H, J=8.1 Hz), 7.33 (br s, 5H), 7.61 (d, 1H, J=7.8 Hz), 7.78 (d, 2H, J=8.1 Hz), 8.01 (s, 1H); ES-MS m/z 570 (M+H).

### EXAMPLE 131

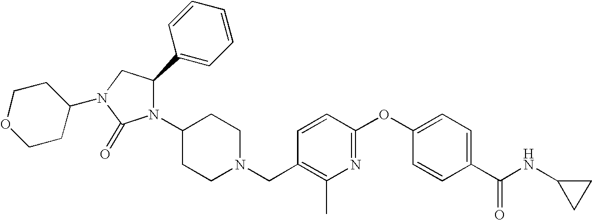

### COMPOUND 131: N-Cyclopropyl-4-(6-methyl-5-{4-[(R)-2-oxo-5-phenyl-3-(tetrahydro-pyran-4-yl)-imidazolidin-1-yl]-piperidin-1-ylmethyl}-pyridin-2-yloxy)-benzamide

Following general procedure F: 4-(6-methyl-5-{4-[(R)-2-oxo-5-phenyl-3-(tetrahydro-pyran-4-yl)-imidazolidin-1-yl]-piperidine-1-ylmethyl}-pyridin-2-yloxy)-benzoic acid (COMPOUND 74) afforded COMPOUND 131 as white solid (40 mg, 48%). ¹H NMR (CDCl₃) δ 0.62 (m, 2H), 0.87 (q, 2H, J=6.6 Hz), 1.21 (m, 1H), 1.40 (d, 1H, J=10.8 Hz), 1.66 (m, 5H), 1.92 (m, 2H), 2.37 (s, 3H), 2.64 (d, 1H, J=11.4 Hz), 2.82 (d, 1H, J=11.4 Hz), 2.90 (sept, 1H, J=3.6 Hz), 3.07 (m, 1H), 3.31 (s, 2H), 3.47 (m, 2H), 3.76 (t, 1H, J=9.0 Hz), 3.76 (m, 1H), 4.02 (m, 3H), 4.59 (m, 1H), 6.18 (s, 1H), 6.60 (d, 1H, J=8.1 Hz), 7.11 (d, 2H, J=8.7 Hz), 7.33 (s, 5H), 7.49 (d, 1H, J=8.4 Hz), 7.72 (d, 2H, J=8.7 Hz); ES-MS m/z 610 (M+H).

### EXAMPLE 132

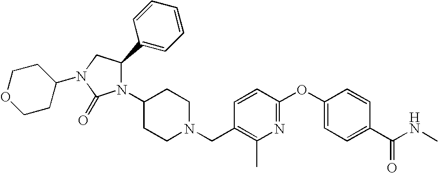

### COMPOUND 132: N-Methyl-4-(6-methyl-5-{4-[(R)-2-oxo-5-phenyl-3-(tetrahydro-pyran-4-yl)-imidazolidin-1-yl]-piperidin-1-ylmethyl}-pyridin-2-yloxy)-benzamide

Following general procedure E: 4-(6-methyl-5-{4-[(R)-2-oxo-5-phenyl-3-(tetrahydro-pyran-4-yl)-imidazolidin-1-yl]-piperidine-1-ylmethyl}-pyridin-2-yloxy)-benzoic acid (COMPOUND 74) afforded COMPOUND 132 as white solid (69 mg, 84%). ¹H NMR (CDCl₃) δ 1.19 (dq, 1H, J=12.0, 3.6 Hz), 1.42 (d, 1H, J=12.0 Hz), 1.66 (m, 5H), 1.92 (m, 2H), 1.99 (m, 1H), 2.38 (s, 3H), 2.64 (d, 1H, J=11.4 Hz), 2.83 (d, 1H, J=11.4 Hz), 3.02 (d, 3H, J=4.8 Hz), 3.07 (m, 1H), 3.31 (s, 2H), 3.46 (m, 2H), 3.65 (t, 1H, J=9.0 Hz), 3.65 (m, 11H), 4.01 (m, 3H), 4.59 (m, 1H), 6.07 (s, 1H), 6.60 (d, 1H, J=8.1 Hz), 7.12 (d, 2H, J=8.4 Hz), 7.33 (s, 5H), 7.49 (d, 1H, J=8.1 Hz), 7.74 (d, 2H, J=8.7 Hz); ES-MS m/z 584 (M+H). Anal. Calcd. for C₃₄H₄₁N₅O₄.0.9CH₂Cl₂: C, 63.50; H, 6.53; N, 10.61. Found: C, 63.46; H, 6.69; N, 10.53.

### EXAMPLE 133

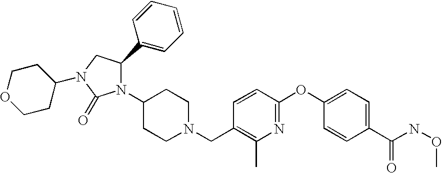

### COMPOUND 133: N-Methoxy-4-(6-methyl-5-{4-[(R)-2-oxo-5-phenyl-3-(tetrahydro-pyran-4-yl)-imidazolidin-1-yl]-piperidin-1-ylmethyl}-pyridin-2-yloxy)-benzamide

Following general procedure E: 4-(6-methyl-5-{4-[(R)-2-oxo-5-phenyl-3-(tetrahydro-pyran-4-yl)-imidazolidin-1-yl]-piperidine-1-ylmethyl}-pyridin-2-yloxy)-benzoic acid (COMPOUND 74) afforded COMPOUND 133 as white solid (39 mg, 46%). ¹H NMR (CDCl₃) δ 1.19 (dq, 1H, J=12.0, 3.6 Hz), 1.42 (d, 1H, J=12.0 Hz), 1.66 (m, 5H), 1.92 (m, 2H), 1.99 (q, 1H, J=11.4 Hz), 2.37 (s, 3H), 2.65 (d, 1H, J=11.4 Hz), 2.82 (d, 1H, J=11.4 Hz), 3.07 (m, 1H), 3.31 (s, 2H), 3.45 (m, 2H), 3.64 (t, 1H, J=9.0 Hz), 3.64 (m, 1H), 3.89 (s, 3H), 4.00 (m, 3H), 4.59 (m, 1H), 6.62 (d, 1H, J=8.1 Hz), 7.12 (d, 2H, J=8.4 Hz), 7.33 (s, 5H), 7.50 (d, 1H, J=8.4 Hz), 7.74 (d, 2H, J=8.7 Hz), 8.69 (s, 1H); ¹³C NMR (CDCl₃) δ 22.14, 29.51, 30.18, 30.54, 31.09, 48.77, 49.13, 52.62, 53.52, 53.65, 56.62, 59.21, 64.65, 67.54, 67.64, 108.96, 120.24 (2C), 127.07 (2C), 128.19, 128.68, 129.27 (2C), 129.36 (2C), 141.45, 142.88, 156.96, 158.41, 160.49, 161.03; ES-MS m/z 600 (M+H). Anal. Calcd. for C₃₄H₄₁N₅O₅.0.3CH₂Cl₂.0.3C₃H₇NO: C, 65.33; H, 6.81; N, 11.47. Found: C, 65.30; H, 6.84; N, 11.46.

### EXAMPLE 134

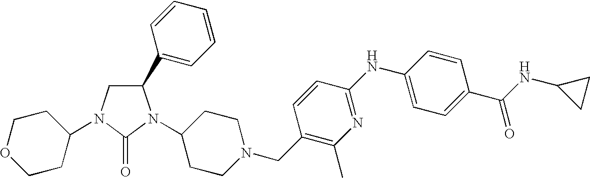

### COMPOUND 134: N-Cyclopropyl-4-(6-methyl-5-{4-[(R)-2-oxo-5-phenyl-3-(tetrahydro-pyran-4-yl)-imidazolidin-1-yl]-piperidin-1-ylmethyl}-pyridin-2-ylamino)-benzamide

Following general procedure E: 4-(6-methyl-5-{4-[(R)-2-oxo-5-phenyl-3-(tetrahydro-pyran-4-yl)-imidazolidin-1-yl]-piperidin-1-ylmethyl}-pyridin-2-ylamino)-benzoic acid (COMPOUND 152) afforded COMPOUND 134 as a yellow foam (45 mg, 44%). ¹H NMR (CDCl₃) δ 0.58-0.63 (m, 2H), 0.83-0.89 (m, 2H), 1.18 (ddd, 1H, J=25.2, 13.2, 4.8 Hz), 1.38-1.42 (m, 1H), 1.57-1.42 (m, 5H), 1.85-2.04 (m, 3H), 2.40 (s, 3H), 2.63-2.67 (m, 1H), 2.82-2.92 (m, 2H), 3.04-3.09 (m, 1H), 3.23-3.33 (m, 2H), 3.43-3.52 (m, 2H), 3.60-3.68 (m, 2H), 3.98-4.10 (m, 3H), 4.59 (dd, 1H, J=9.0, 6.6 Hz), 6.15 (br s, 11H), 6.59 (s, 1H), 6.69 (d, 1H), 7.33-7.41 (m, 8H), 7.68 (d, 2H, J=8.7 Hz); ¹³C NMR (CDCl₃) δ 6.77, 21.96, 23.07, 29.18, 29.82, 30.19, 30.82, 48.39, 48.71, 52.31, 53.03, 53.18, 56.18, 59.19, 67.18, 67.28, 106.78, 117.24, 124.00, 126.59, 126.69, 128.23, 128.83, 139.43, 142.63, 144.35, 152.75, 156.42, 160.08, 168.48; ES-MS m/z 609 (M+1). Anal. Calcd. for C₃₆H₄₄N₆O₃.0.2CH₂Cl₂.0.9CH₄O: C, 68.07; H, 7.39; N, 12.84. Found: C, 67.80; H, 7.35; N, 12.74.

### EXAMPLE 135

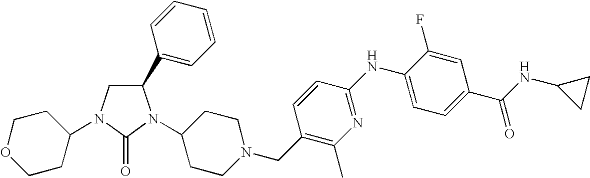

### COMPOUND 135: N-Cyclopropyl-3-fluoro-4-(6-methyl-5-{4-[(R)-2-oxo-5-phenyl-3-(tetrahydro-pyran-4-yl)-imidazolidin-1-yl-piperidin-1-ylmethyl}-pyridin-2-yloxy)-benzamide

Following general procedure E: 3-fluoro-4-(6-methyl-5-{4-[(R)-2-oxo-5-phenyl-3-(tetrahydro-pyran-4-yl)-imidazolidin-1-yl]-piperidin-1-ylmethyl}-pyridin-2-yloxy)-benzoic acid (COMPOUND 105) afforded COMPOUND 135 as a yellow foam (24 mg, 32%). ¹H NMR (CDCl₃) δ 0.60-0.65 (m, 2H), 0.85-0.92 (m, 2H), 1.11-1.25 (m, 1H), 1.39-1.43 (m, 1H), 1.61-1.72 (m, 5H), 1.82-2.08 (m, 3H), 2.29 (s, 3H), 2.61-2.65 (m, 1H), 2.79-2.83 (m, 1H), 2.86-2.94 (m, 1H), 3.06 (dd, 1H, J=8.4, 6.9 Hz), 3.24-3.33 (m, 2H), 3.41-3.52 (m, 2H), 3.59-3.68 (m, 2H), 3.97-4.10 (m, 3H), 4.59 (dd, 1H, J=9.0, 6.6 Hz), 6.66 (d, 1H, J=9.3 Hz), 7.19-7.24 (m, 1H), 7.29-7.37 (m, 5H), 7.48-7.50 (m, 2H), 7.58 (dd, 1H, J=10.8, 1.8 Hz); ¹³C NMR (CDCl₃) δ 6.75, 21.72, 23.25, 29.15, 29.83, 30.19, 30.79, 48.40, 48.73, 52.24, 53.12, 53.29, 56.20, 58.86, 67.20, 67.30, 107.26, 115.74, 116.01, 123.00, 123.27, 126.69, 127.43, 128.25, 128.85, 140.99, 142.62, 144.36, 144.52, 156.07, 160.05, 160.38, 167.28; ES-MS m/z 628 (M+1). Anal. Calcd. for C₃₆H₄₂N₅FO_{4.}0.7CH₄O: C, 67.80; H, 6.94; N, 10.77. Found: C, 67.84; H, 6.84; N, 10.72.

### EXAMPLE 136

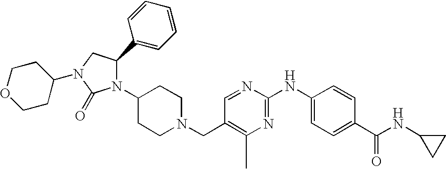

### COMPOUND 136: N-Cyclopropyl-4-(4-methyl-5-{4-[(R)-2-oxo-5-phenyl-3-(tetrahydro-pyran-4-yl)-imidazolidin-1-yl]-piperidin-1-ylmethyl}-pyrimidin-2-ylamino)-benzamide

Following general procedure E: 4-(4-methyl-5-{4-[(R)-2-oxo-5-phenyl-3-(tetrahydro-pyran-4-yl)-imidazolidin-1-yl]-piperidin-1-ylmethyl}-pyrimidin-2-ylamino)-benzoic acid (COMPOUND 107) afforded COMPOUND 136 as an orange powder (32.1 mg, 60%). ¹H NMR (CDCl₃) δ 0.60-0.63 (m, 2H), 0.82-0.88 (m, 2H), 1.35-1.47 (m, 1H), 1.41 (d, 1H, J=12.3 Hz), 1.64-2.00 (m, 8H), 2.39 (s, 3H), 2.64 (d, 1H, J=11.1 Hz), 2.81 (d, 1H, J=12.3 Hz), 2.86-2.92 (m, 1H), 3.06 (dd, 1H, J=8.4, 6.9 Hz), 3.25 (s, 2H), 3.40-3.51 (m, 2H), 3.58-3.63 (m, 1H), 3.64 (t, 2H, J=9.0 Hz), 3.97-4.04 (m, 3H), 4.57 (dd, 1H, J=8.1, 6.6 Hz), 6.25 (d, 1H, J=2.4 Hz), 7.27-7.37 (m, 6H), 7.66-7.73 (m, 4H), 8.10 (s, 1H); ¹³C NMR (CDCl₃) δ 6.8, 21.8, 23.1, 29.1, 29.8, 30.2, 30.7, 48.3, 48.7, 52.2, 52.9, 53.1, 56.3, 56.8, 67.2, 67.3, 117.7, 121.3, 126.7, 127.3, 128.0, 128.3, 128.9, 142.5, 143.0, 157.9, 158.5, 160.0, 168.1, 168.4; ES-MS m/z 610 (M+H). Anal. Calcd. for C₃₅H₄₃N₇O_{3.}0.8CH₂Cl₂: C, 63.45; H, 6.63; N, 14.47. Found: C, 63.32; H. 6.66; N, 14.41.

### EXAMPLE 137

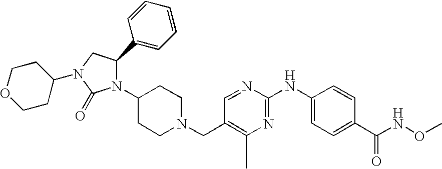

### COMPOUND 137: N-Methoxy-4-(4-methyl-5-{4-[(R)-2-oxo-5-phenyl-3-(tetrahydro-pyran-4-yl)-imidazolidin-1-yl]-piperidin-1-ylmethyl}-pyrimidin-2-ylamino)-benzamide

Following general procedure E: 4-(4-methyl-5-{4-[(R)-2-oxo-5-phenyl-3-(tetrahydro-pyran-4-yl)-imidazolidin-1-yl]-piperidin-1-ylmethyl}-pyrimidin-2-ylamino)-benzoic acid (COMPOUND 107) afforded COMPOUND 137 as a pale orange powder (28.9 mg, 55%). ¹H NMR (CDCl₃) δ 1.20-1.31 (m, 1H), 1.44 (d, 1H, J=10.8 Hz), 1.63-1.76 (m, 5H), 1.85-1.98 (m, 3H), 2.39 (s, 3H), 2.67 (d, 1H, J=10.5 Hz), 2.82 (d, 1H, J=4.8 Hz), 3.07 (t, 1H, J=7.8 Hz), 3.27 (s, 2H), 3.42-3.52 (m, 3H), 3.64 (t, 1H, J=9.0 Hz), 3.88 (s, 3H), 3.96-4.08 (m, 3H), 4.58 (dd, 1H, J=9.0, 6.0 Hz), 7.28-7.37 (m, 5H), 7.71 (d, 2H, J=8.7 Hz), 7.76 (d, 2H, J=8.7 Hz), 8.15 (s, 1H), 9.42 (br s, 1H); ¹³C NMR (CDCl₃) δ 21.9, 29.3, 29.7, 30.3, 48.3, 48.6, 50.8, 52.4, 53.0, 53.1, 56.7, 64.4, 67.1, 67.3, 117.9, 121.4, 124.6, 126.8, 128.3, 128.4, 128.9, 142.2, 143.6, 157.9, 158.5, 160.1, 166.2, 168.0; ES-MS m/z 600 (M+H). Anal. Calcd. for C₃₃H₄₁N₇O₄.0.6CH₂Cl₂: C, 62.02; H, 6.54; N, 15.07. Found: C, 62.40; H, 6.64; N, 14.88.

### EXAMPLE 138

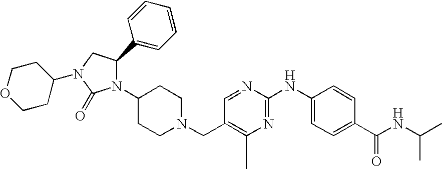

### COMPOUND 138: N-Isopropyl-4-(4-methyl-5-{4-[(R)-2-oxo-5-phenyl-3-(tetrahydro-pyran-4-yl)-imidazolidin-1-yl]-piperidin-1-ylmethyl}-pyrimidin-2-ylamino)-benzamide

Following general procedure E: 4-(4-methyl-5-{4-[(R)-2-oxo-5-phenyl-3-(tetrahydro-pyran-4-yl)-imidazolidin-1-yl]-piperidin-1-ylmethyl}-pyrimidin-2-ylamino)-benzoic acid (COMPOUND 107) afforded COMPOUND 138 as an orange powder (25.0 mg, 58%). ¹H NMR (CDCl₃) δ 1.15-1.26 (m, 1H), 1.25 (d, 6H, J=6.6 Hz), 1.41 (d, 1H, J=11.4 Hz), 1.60-1.71 (m, 4H), 1.86-2.01 (m, 4H), 2.39 (s, 3H), 2.65 (d, 1H, J=10.5 Hz), 2.82 (d, 1H, J=9.9 Hz), 3.07 (t, 1H, J=7.6 Hz), 3.26 (s, 2H), 3.42-3.50 (m, 2H), 3.58-3.64 (m, 1H), 3.64 (t, 1H, J=9.0 Hz), 3.97-4.07 (m, 3H), 4.22-4.33 (m, 1H), 4.58 (dd, 1H, J=9.3, 6.9 Hz), 5.89 (m, 1H), 7.27-7.37 (m, 5H), 7.69 (d, 2H, J=9.0 Hz), 7.72 (d, 2H, J=9.0 Hz), 8.10 (s, 1H); ¹³C NMR (CDCl₃) δ 20.8, 21.9, 28.0, 28.8, 29.2, 29.7, 40.7, 47.3, 47.7, 51.1, 51.9, 52.1, 55.2, 55.8, 66.2, 66.3, 116.8, 120.3, 125.7, 126.8, 127.3, 127.8, 141.5, 141.7, 156.9, 157.5, 159.0, 165.2, 167.1; ES-MS m/z 612 (M+H). Anal. Calcd. for C₃₅H₄₅N₇O₃.0.8CH₂Cl₂: C, 63.26; H, 6.91; N, 14.42. Found: C, 63.56; H, 6.96; N, 14.34.

### EXAMPLE 139

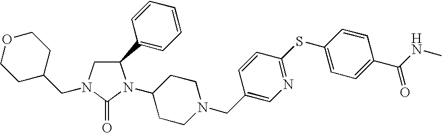

### COMPOUND 139: N-Methyl-4-(5-{4-[(R)-2-oxo-5-phenyl-3-(tetrahydro-pyran-4-ylmethyl)-imidazolidin-1-yl]-piperidin-1-ylmethyl}-pyridin-2-ylsulfanyl)-benzamide

Following general procedure E: 4-(5-{4-[(R)-2-oxo-5-phenyl-3-(tetrahydro-pyran-4-ylmethyl)-imidazolidin-1-yl]-piperidin-1-ylmethyl}-pyridin-2-ylsulfanyl)-benzoic acid (COMPOUND 89) afforded COMPOUND 139 as a colourless foam (80 mg, 74%). ¹H NMR (CDCl₃) δ 1.10-1.41 (m, 4H), 1.55-1.69 (m, 3H), 1.69-2.03 (m, 4H), 2.61-2.65 (m, 1H), 2.80-2.83 (m, 1H), 3.02 (d, 3H, J=4.8 Hz), 3.05-3.15 (m, 3H), 3.30-3.38 (m, 4H), 3.61-3.69 (m, 2H), 3.94-3.98 (m, 2H), 4.59 (dd, 1H, J=9.3, 6.6 Hz), 6.17-6.19 (m, 1H), 6.96 (d, 1H, J=8.4 Hz), 7.28-7.42 (m, 6H), 7.56 (d, 2H, J=8.4 Hz), 7.75 (d, 2H, J=8.4 Hz), 8.29 (d, 1H, J=1.8 Hz); ¹³C NMR (CDCl₃) δ 26.92, 29.00, 30.69, 30.74, 30.88, 34.06, 50.07, 52.14, 52.99, 53.16, 53.93, 55.96, 59.37, 67.60, 122.50, 126.64, 127.85, 128.23, 128.88, 131.30, 133.42, 134.48, 136.14, 137.73, 142.64, 150.31, 157.75, 161.17, 167.51; ES-MS m/z 600 (M+1). Anal. Calcd. for C₃₄H₄₁N₅SO₃.0.3CH₂Cl₂.0.1H₂O: C, 65.70; H, 6.72; N, 11.17. Found: C, 65.69; H, 6.71; N, 11.15.

### EXAMPLE 140

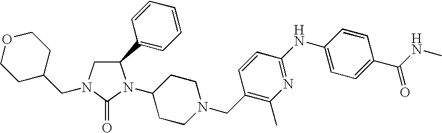

### COMPOUND 140: N-Methyl-4-(6-methyl-5-{4-[(R)-2-oxo-5-phenyl-3-(tetrahydro-pyran-4-ylmethyl)-imidazolidin-1-yl]-piperidin-1-ylmethyl}-pyridin-2-ylamino)-benzamide

Following general procedure E: 4-(6-methyl-5-{4-[(R)-2-oxo-5-phenyl-3-(tetrahydro-pyran-4-ylmethyl)-imidazolidin-1-yl]-piperidin-1-ylmethyl}-pyridin-2-ylamino)-benzoic acid (COMPOUND 156) afforded COMPOUND 140 as a yellow foam (64 mg, 78%). ¹H NMR (CDCl₃) δ 1.11-1.42 (m, 4H), 1.55-2.04 (m, 7H), 2.40 (s, 3H), 2.63-2.67 (m, 1H), 2.81-2.85 (m, 1H), 3.00 (d, 3H, J=4.8 Hz), 3.03-3.16 (m, 3H), 3.27-3.38 (m, 4H), 3.61-3.69 (m, 2H), 3.94-3.97 (m, 2H), 4.60 (dd, 1H, J=9.3, 6.6 Hz), 6.08-6.10 (m, 1H), 6.63 (s, 1H), 6.69 (d, 1H, J=8.1 Hz), 7.28-7.39 (m, 8H), 7.70 (d, 2H, J=8.7 Hz); ¹³C NMR (CDCl₃) δ 21.96, 26.78, 29.20, 30.71, 30.75, 30.88, 34.07, 50.09, 52.42, 53.03, 53.19, 53.94, 56.02, 59.20, 67.59, 106.74, 117.34, 123.95, 126.65, 126.90, 128.20, 128.84, 139.46, 142.67, 144.21, 152.81, 156.41, 161.21, 167.83; ES-MS m/z 597 (M+1). Anal. Calcd. for C₃₅H₄₄N₆O₃.0.6CH₂Cl₂.0.1CH₄O: C, 65.87; H, 7.06; N, 12.91. Found: C, 65.78; H, 7.00; N, 12.85.

### EXAMPLE 141

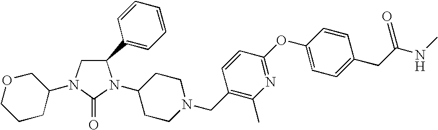

### COMPOUND 141: N-Methyl-2-[4-(6-methyl-5-{4-[(R)-2-oxo-5-phenyl-3-(tetrahydro-pyran-4-yl)-imidazolidin-1-yl]-piperidin-1-ylmethyl}-pyridin-2-yloxy)-phenyl]-acetamide

Following general procedure E: [4-(6-methyl-5-{4-[(R)-2-oxo-5-phenyl-3-(tetrahydro-pyran-4-yl)-imidazolidin-1-yl]-piperidin-1-ylmethyl}-pyridin-2-yloxy)-phenyl]-acetic acid (COMPOUND 88) afforded COMPOUND 141 as a colourless foam (61 mg, 79%). ¹H NMR (CDCl₃) δ 1.18 (ddd, 1H, J=24.3, 12.0, 3.6 Hz), 1.39-1.43 (m, 1H), 1.58-1.76 (m, 5H), 1.83-2.06 (m, 3H), 2.38 (s, 3H), 2.62-2.66 (m, 1H), 2.77-2.84 (m, 4H), 3.06 (dd, 1H, J=8.4, 6.9 Hz), 3.25-3.35 (m, 2H), 3.41-3.52 (m, 2H), 3.57 (s, 2H), 3.60-3.68 (m, 2H), 3.97-4.10 (m, 3H), 4.59 (dd, 1H, J=9.3, 6.9 Hz), 5.43 (br s, 1H), 6.55 (d, 1H, J=8.4 Hz), 7.07-7.10 (m, 2H), 7.22-7.25 (m, 2H), 7.28-7.37 (m, 5H), 7.46 (d, 1H, J=8.1 Hz); ¹³C NMR (CDCl₃) δ 22.22, 26.89, 29.52, 30.19, 30.56, 31.14, 43.35, 48.77, 49.10, 52.61, 53.51, 53.63, 56.58, 59.28, 67.57, 67.67, 108.13, 121.25, 127.08, 127.52, 128.63, 129.23, 131.00, 131.08, 141.30, 142.98, 154.42, 156.88, 160.43, 161.81, 172.03; ES-MS m/z 598 (M+1). Anal. Calcd. for C₃₅H₄₃N₅O₄.0.2CH₂Cl₂.0.1H₂O: C, 68.57; H, 7.13; N, 11.36. Found: C, 68.56; H, 7.17; N, 11.16.

### EXAMPLE 142

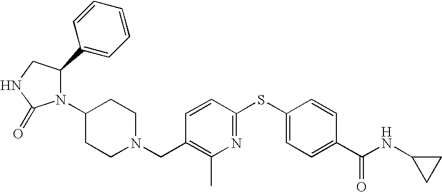

### COMPOUND 142: N-Cyclopropyl-4-{6-methyl-5-[4-((R)-2-oxo-5-phenyl-imidazolidin-1-yl)-piperidin-1-ylmethyl]-pyridin-2-ylsulfanyl}-benzamide

Following general procedure E: 4-{6-methyl-5-[4-((R)-2-oxo-5-phenyl-imidazolidin-1-yl)-piperidin-1-ylmethyl]-pyridin-2-ylsulfanyl}-benzoic acid (COMPOUND 100) (45.8 mg, 0.0912 mmol) gave COMPOUND 142 as a white powder (38.5 mg, 78%). ¹H NMR (CDCl₃) δ 0.59-0.64 (m, 2H), 0.82-0.89 (m, 2H), 1.08-1.25 (m, 1H), 1.39 (d, 1H, J=11.4 Hz), 1.63 (d, 1H, J=9.9 Hz), 1.85-2.03 (m, 3H), 2.42 (s, 3H), 2.60 (d, 1H, J=10.2 Hz), 2.78 (d, 1H, J=9.6 Hz), 2.86-2.92 (m, 1H), 3.20 (t, 1H, J=7.5 Hz), 3.27 (s, 2H), 3.56-3.65 (m, 1H), 3.73 (t, 1H, J=9.0 Hz), 4.70 (dd, 1H, J=9.3, 6.6 Hz), 4.80 (s, 1H), 6.45 (s, 1H), 6.72 (m, 1H), 7.27-7.34 (m, 6H), 7.51 (d, 2H, J=8.4 Hz), 7.71 (d, 2H, J=8.4 Hz); ¹³C NMR (CDCl₃) δ 7.1, 22.4, 23.6, 29.5, 31.2, 48.6, 52.2, 53.5, 53.7, 59.0, 59.5, 120.5, 127.2, 128.2, 128.7, 129.2, 129.8, 133.5, 134.4, 137.2, 138.5, 142.7, 156.8, 158.7, 162.9, 168.6; ES-MS m/z 542 (M+H). Anal. Calcd. for C₃₁H₃₅N₅O₂S.1.0CH₂Cl₂: C, 61.34; H, 5.95; N, 11.18. Found: C, 61.43; H, 5.92; N, 11.15.

### EXAMPLE 143

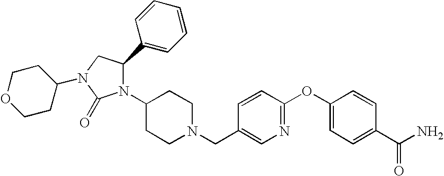

### COMPOUND 143: 4-(5-{4-[(R)-2-Oxo-5-phenyl-3-(tetrahydro-pyran-4-yl)-imidazolidin-1-yl]-piperidin-1-ylmethyl}-pyridin-2-yloxy)-benzamide

To a solution of 4-(5-{4-[(R)-2-oxo-5-phenyl-3-(tetrahydro-pyran-4-yl)-imidazolidin-1-yl]-piperidin-1-ylmethyl}-pyridin-2-yloxy)-benzonitrile (COMPOUND 50) (0.135 g, 0.252 mmol) in TFA (3 mL) was added 6 drops concentrated H₂SO₄. The mixture was stirred and heated at reflux overnight. After cooled to room temperature the mixture was neutralized with 10 N NaOH and extracted with CH₂Cl₂ (3×10 mL). The combined extract was dried over anhydrous Na₂SO₄. After filtration the solvent was removed, and the residue was purified by flash chromatography on silica gel (CH₂Cl₂/MeOH, 10:1 in v/v) to give COMPOUND 143 as an off-white solid (0.065 g, 41%). ¹H NMR (CDCl₃) δ 1.17-1.23 (m, 1H), 1.39-1.44 (m, 1H), 1.60-1.70 (m, 5H), 1.84-2.01 (m, 3H), 2.64-2.69 (m, 1H), 2.82-2.86 (m, 1H), 3.04 (dd, 1H, J=6.9, 8.4 Hz), 3.34-3.44 (m, 2H), 3.45-3.52 (m, 2H), 3.60-3.68 (m, 2H), 3.96-4.07 (m, 3H), 4.59 (dd, 1H, J=6.9, 9.3 Hz), 5.68 (br s, 1H), 6.06 (br s, 1H), 6.88 (d, 1H, J=8.4 Hz), 7.14-7.20 (m, 2H), 7.30-7.37 (m, 5H), 7.62 (dd, 1H, J=2.4, 8.4 Hz), 7.81-7.87 (m, 2H), 8.01 (d, 1H, J=2.4 Hz); ¹³C NMR (CDCl₃) δ 29.12, 29.93, 30.30, 30.84, 48.55, 48.86, 52.24, 53.07, 53.20, 56.34, 59.28, 67.30, 67.40, 111.87, 120.73, 126.82, 128.39, 129.00, 129.38, 129.52, 140.85, 142.71, 147.88, 157.61, 160.20, 162.25, 169.01; ES-MS m/z 556 (M+1). Anal. Calcd. for C₃₂H₃₇N₅O₄.0.8CH₂Cl₂: C, 63.17; H, 6.24; N, 11.23. Found: C, 63.12; H, 6.19; N, 11.37.

### EXAMPLE 144

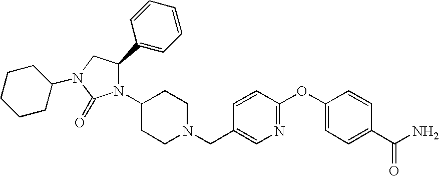

### COMPOUND 144: 4-{5-[4-((R)-3-Cyclohexyl-2-oxo-5-phenyl-imidazolidin-1-yl)-piperidin-1-ylmethyl]-pyridin-2-yloxy}-benzamide

To a solution of 4-{5-[4-((R)-3-cyclohexyl-2-oxo-5-phenyl-imidazolidin-1-yl)-piperidin-1-ylmethyl]-pyridin-2-yloxy}-benzonitrile (COMPOUND 54) (77 mg, 0.14 mmol) in TFA (2 mL) was added c. H₂SO₄ (4 drops). The reaction was heated to 100° C. for 16 h, and then concentrated to dryness under reduced pressure. The crude residue was then purified by column chromatography on silica gel (NH₃/Et₂O) to afford COMPOUND 144 as a white solid (52 mg, 67%). ¹H NMR (CDCl₃) δ 1.04 (q, 1H, J=10.8 Hz), 1.15-1.45 (m, 6H), 1.63 (d, 2H, J=12.3 Hz), 1.75 (m, 4H), 1.84-2.05 (m, 3H), 2.67 (d, 1H, J=10.8 Hz), 2.81 (d, 1H, J=10.8 Hz), 3.05 (m, 1H), 3.36 (s, 2H), 3.60 (t, 1H, J=9.0 Hz), 3.63 (m, 1H), 3.75 (m, 1H), 4.55 (m, 1H), 6.88 (d, 1H, J=9.0 Hz), 7.18 (d, 2H, J=9.0 Hz), 7.33 (br s, 5H), 7.63 (d, 1H, J=9.0 Hz), 7.85 (d, 2H, J=9.0 Hz), 8.01 (s, 1H); ES-MS m/z 554 (M+H).

### EXAMPLE 145

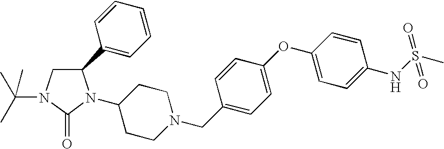

### COMPOUND 145: N-(4-{4-[4-((R)-3-tert-Butyl-2-oxo-5-phenyl-imidazolidin-1-yl)-piperidin-1-ylmethyl]-phenoxy}-phenyl)-methanesulfonamide

A mixture of 4-hydroxybenzaldehyde (3.0 g, 25 mmol), 1-fluoro-4-nitrobenzene (2.35 mL, 22.5 mmol) and K₂CO₃ (10.4 g, 75 mmol) was heated at 80° C. for 3 days. Aqueous work-up afforded the crude aldehyde. The aldehyde (415 mg) was subsequently reduced with NaBH₄ (65 mg, 1.7 mmol) in MeOH (17 mL) to afford [4-(4-nitro-phenoxy)-phenyl]-methanol (424 mg, quant).

To a solution of the above alcohol (418 mg, 1.71 mmol) and Et₃N (0.26 mL, 1.9 mmol) in CH₂Cl₂ (17 mL) at 0° C. was added MsCl (0.13 mL, 1.7 mmol) and the mixture was stirred at 0° C. for 20 minutes. Aqueous work-up afforded the desired crude mesylate (592 mg). A solution of the mesylate (282 mg, 0.87 mmol), (R)-1-tert-butyl-4-phenyl-3-piperidin-4-yl-imidazolidin-2-one (219 mg, 0.727 mmol) and DIPEA (0.20 mL, 1.1 mmol) in CH₃CN (4.8 mL) was heated at 60° C. for 18 hours. Standard work up and purification afforded the desired product as a colourless foam (200 mg). To a solution of the above compound (200 mg) in MeOH (10 mL) in a Parr flask was added 10% Pd/C (30 mg) under N₂. The mixture was hydrogenated at room temperature under 45 psi H₂ for 1 hour and after purification afforded (R)-3-{1-[4-(4-amino-phenoxy)-benzyl]-piperidin-4-yl}-1-tert-butyl-4-phenyl-imidazolidin-2-one (127 mg, 35% over 2 steps).

A solution of the above aniline (53 mg, 0.11 mmol), MsCl (0.010 mL, 0.13 mmol), Et₃N (0.022 mL, 0.16 mmol) and DMAP (catalytic) in DCE (1.1 mL) was heated at 50° C. for 19 hours. Standard work-up and purification afforded COMPOUND 145 as a yellow foam (17 mg, 28%). ¹H NMR (CD₃OD) δ 1.09-1.23 (m, 1H), 1.36 (s, 9H), 1.37-1.43 (m, 1H), 1.63-1.67 (m, 1H), 1.78-1.91 (m, 2H), 1.95-2.02 (m, 1H), 2.66-2.70 (m, 1H), 2.87 (d, 1H, J=11.1 Hz), 2.99 (s, 3H), 3.06 (dd, 1H, J=8.7, 7.2 Hz), 3.31-3.41 (m, 2H), 3.63-3.72 (m, 2H), 4.49 (dd, 1H, J=8.1, 7.2 Hz), 6.88-6.90 (m, 2H), 6.94-6.99 (m, 2H), 7.17-7.21 (m, 4H), 7.28-7.35 (m, 5H); ¹³C NMR (CD₃OD) δ 27.47, 28.87, 31.05, 39.17, 50.89, 52.11, 53.15, 53.26, 55.33, 62.15, 118.62, 119.57, 123.96, 126.82, 128.05, 128.71, 130.53, 131.49, 133.66, 142.92, 155.54, 155.73, 161.13; ES-MS m/z 577 (M+1). Anal. Calcd. for C₃₂H₄₀N₄SO₄.0.3CH₂Cl₂: C, 64.42; H, 6.79; N, 9.30. Found: C, 64.68; H, 6.81; N, 9.34.

### EXAMPLE 146

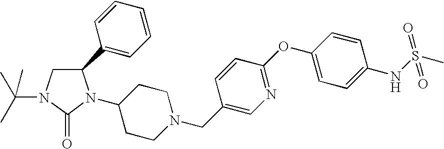

### COMPOUND 146: N-(4-{5-[4-((R)-3-tert-Butyl-2-oxo-5-phenyl-imidazolidin-1-yl)-piperidin-1-ylmethyl]-pyridin-2-yloxy}-phenyl)-methanesulfonamide

A mixture of 2-bromo-5-methylpyridine (876 mg, 5.09 mmol), 4-nitrophenol (354 mg, 2.54 mmol) and K₂CO₃ (352 mg, 2.55 mmol) in DMSO (1.3 mL) was heated at 190° C. for 12 hours. Aqueous work-up and purification afforded the desired product as yellow crystals (111 mg, 19%).

A solution of the above substrate (140 mg, 0.608 mmol), NBS (130 mg, 0.730 mmol) and (BzO)₂ (15 mg, 0.062 mmol) in CCl₄ (1.5 mL) was heated to reflux for 3 hours. Standard work-up and purification afforded 5-bromomethyl-2-(4-nitro-phenoxy)-pyridine (57 mg, 30%).

A solution of the bromide (107 mg, 0.346 mmol), (R)-1-tert-butyl-4-phenyl-3-piperidin-4-yl-imidazolidin-2-one (125 mg, 0.415 mmol) and DIPEA (0.084 mL, 0.48 mmol) in CH₃CN (2.3 mL) was heated at 60° C. for 22 hours. Standard work up and purification afforded the desired product as a yellow foam (142 mg, 78%). To a solution of the above compound (129 mg, 0.244 mmol) in MeOH (8 mL) in a Parr flask was added 10% Pd/C (20 mg) under N₂. The mixture was hydrogenated at room temperature under 45 psi H₂ for 1.5 hours and after purification afforded (R)-3-{1-[6-(4-amino-phenoxy)-pyridin-3-ylmethyl]-piperidin-4-yl}-1-tert-butyl-4-phenyl-imidazolidin-2-one (95 mg, 78%).

To solution of the above aniline (95 mg, 0.19 mmol) and Et₃N (0.027 mL, 0.19 mmol) in CH₂Cl₂ (1.9 mL) at 0° C. was added a solution of MsCl (0.010 mL, 0.13 mmol) in CH₂Cl₂ (2 mL) dropwise. The mixture was stirred at 0° C. for 15 minutes. Standard work-up and purification afforded COMPOUND 146 as a colourless foam (21 mg, 28%). ¹H NMR (CDCl₃) δ 1.06-1.20 (m, 1H), 1.36 (s, 9H), 1.37-1.41 (m, 1H), 1.59-1.66 (m, 1H), 1.76-1.89 (m, 2H), 1.95-2.02 (m, 2H), 2.64 (d, 1H, J=9.9 Hz), 2.83 (d,1H, J=10.8 Hz), 3.02 (s, 3H), 3.07 (dd, 1H, J=7.5, 7.5 Hz), 3.28-3.38 (m, 2H), 3.63-3.70 (m, 2H), 4.48 (dd, 1H, J=8.7, 7.2 Hz), 6.44 (br s, 1H), 6.84 (d, 1H, J=8.4 Hz), 7.10-7.14 (m, 2H), 7.23-7.35 (m, 7H), 7.59 (dd, 1H, J=8.4, 2.4 Hz), 7.97 (d, 1H, J=2.1 Hz); ¹³C NMR (CDCl₃) δ 27.46, 28.80, 30.94, 39.29, 50.84, 52.00, 53.02, 53.17, 55.44, 59.18, 111.20, 122.15, 123.05, 126.86, 128.11, 128.74, 128.93, 132.99, 140.62, 142.76, 147.64, 151.93, 161.08, 162.60; ES-MS m/z 578 (M+1). Anal. Calcd. for C₃₁H₃₉N₅SO₄.0.2CH₂Cl₂: C, 63.01; H, 6.68; N, 11.78. Found: C, 62.76; H, 6.73; N, 11.47.

### EXAMPLE 147

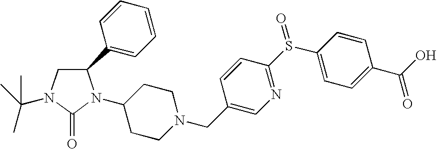

### COMPOUND 147: 4-{5-[4-((R)-3-tert-Butyl-2-oxo-5-phenyl-imidazolidin-1-yl)-piperidin-1-ylmethyl]-pyridine-2-sulfinyl}-benzoic acid

Following general procedure G: a solution of (R)-1-tert-butyl-4-phenyl-3-piperidin-4-yl-imidazolidin-2-one (69 mg, 0.23 mmol), 4-(5-bromomethyl-pyridin-2-ylsulfanyl)-benzoic acid methyl ester (77 mg, 0.23 mmol) and N,N-diisopropylethylamine (0.1 mL) in CH₃CN (2.3 mL) was heated to 60° C. overnight. Standard work-up and purification afforded the ester (50 mg, 39%).

To a solution of the above ester (50 mg, 0.089 mmol) in MeOH (2 mL) at 0° C. was added 1N HCl (0.3 mL) followed by a solution of OXONE® (83 mg, 0.13 mmol) in H₂O (0.44 mL). Standard work-up afforded the desired 4-{5-[4-((R)-3-tert-butyl-2-oxo-5-phenyl-imidazolidin-1-yl)-piperidin-1-ylmethyl]-pyridine-2-sulfinyl}-benzoic acid methyl ester. Following general procedure H, the above methyl ester and a mixture of NaOH and LiOH afforded COMPOUND 147 as a white solid (36 mg, 71% over 2 steps). ¹H NMR (CD₃OD) δ 1.35 (s, 9H), 1.55-1.84 (m, 3H), 2.10-2.21 (m, 1H), 2.75-2.87 (m, 2H), 3.11-3.23 (m, 2H), 3.32-3.35 (m, 1H), 3.56-3.63 (m, 1H), 3.77 (t, 1H, J=9.0 Hz), 4.16 (s, 2H), 4.55-4.61 (m, 1H), 7.27-7.37 (m, 5H), 7.86-7.90 (m, 2H), 7.03-8.11 (m, 4H), 8.64 (s, 1H); ES-MS m/z 561 (M+1).

### EXAMPLE 148

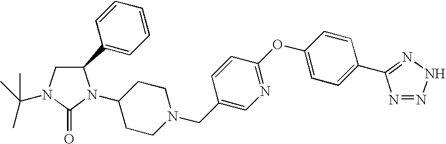

### COMPOUND 148: (R)-1-tert-Butyl-4-phenyl-3-(1-{6-[4-(2H-tetrazol-5-yl)-phenoxy]-pyridin-3-ylmethyl}-piperidin-4-yl)-imidazolidin-2-one

Following general procedure G: a solution of (R)-1-tert-butyl-4-phenyl-3-piperidin-4-yl-imidazolidin-2-one (150 mg, 0.498 mmol), 4-(5-bromomethyl-pyridin-2-yloxy)-benzonitrile (144 mg, 0.498 mmol) and N,N-diisopropylethylamine (0.14 mL, 0.747 mmol) in CH₃CN (5.0 mL) was heated to 60° C. overnight. Standard work-up and purification afforded 4-{5-[4-((R)-3-tert-butyl-2-oxo-5-phenyl-imidazolidin-1-yl)-piperidin-1-ylmethyl]-pyridin-2-yloxy}-benzonitrile (123 mg). A solution of the above nitrile, NH₄Cl (65 mg, 1.2 mmol) and NaN₃ (78 mg, 1.2 mmol) in DMF (2 mL) was heated to 130° C. for 4 days. Standard work-up and purification afforded COMPOUND 148 as a yellow solid (37 mg, 13% over 2 steps). ¹H NMR (CD₃OD) δ 1.35 (s, 9H), 1.63-1.92 (m, 3H), 2.22-2.33 (m, 1H), 2.89-3.01 (m, 1H), 3.12-3.18 (m, 1H), 3.35-3.38 (m, 1H), 3.45-3.49 (m, 1H), 3.59-3.67 (m, 1H), 3.78 (t, 1H, J=9.0 Hz), 4.20 (s, 2H), 4.55-4.61 (m, 1H), 7.06 (d, 1H, J=8.7 Hz), 7.24 (d, 2H, J=9.3 Hz), 7.28-7.39 (m, 5H), 7.89 (dd, 1H, J=8.4, 2.1 Hz), 8.05 (d, 2H, J=8.7 Hz), 8.17 (d, 1H, J=1.8 Hz); ES-MS m/z 553 (M+1). Anal. Calcd. for C₃₁H₃₆N₈O₂.1.74H₂O.0.31CH₂Cl₂: C, 61.59; H, 6.62; N, 18.35. Found: C, 61.48; H, 6.45; N, 18.66.

### EXAMPLE 149

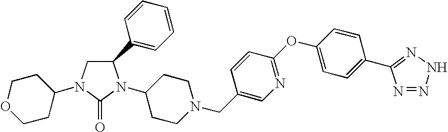

### COMPOUND 149: (R)-4-Phenyl-1-(tetrahydro-pyran-4-yl)-3-(1-{6-[4-(2H-tetrazol-5-yl)-phenoxy]-pyridin-3-ylmethyl}-piperidin-4-yl)-imidazolidin-2-one

Following general procedure A, (R)-4-phenyl-3-piperidin-4-yl-1-(tetrahydro-pyran-4-yl)-imidazolidin-2-one (313 mg, 0.951 mmol) and 4-(5-formyl-6-methyl-pyridin-2-yloxy)-benzonitrile (214 mg, 0.955 mmol) afforded 4-(5-{4-[(R)-2-oxo-5-phenyl-3-(tetrahydro-pyran-4-yl)-imidazolidin-1-yl]-piperidin-1-ylmethyl}-pyridin-2-yloxy)-benzonitrile (376 mg, 74%).

A solution of the above nitrile (150 mg, 0.278 mmol), NH₄Cl (60 mg, 1.1 mmol) and NaN₃ (54 mg, 0.83 mmol) in DMF (2 mL) was heated to 100° C. for 4 hours. Standard work-up and purification afforded COMPOUND 149 as a white solid (153 mg, 95%). ¹H NMR (CD₃OD) δ 1.61-1.92 (m, 7H), 2.27-2.40 (m, 1H), 2.85-2.96 (m, 1H), 3.13-3.19 (m, 1H), 3.47-3.57 (m, 4H), 3.76-3.82 (m, 1H), 3.89-3.98 (m, 3H), 4.19 (s, 2H), 4.70-4.76 (m, 2H), 7.10 (d, 1H, J=8.4 Hz), 7.28 (d, 2H, J=9.0 Hz), 7.36-7.41 (m, 5H), 7.90 (dd, 1H, J=8.4, 2.4 Hz), 8.08 (d, 2H, J=8.7 Hz), 8.18 (d, 1H, J=2.4 Hz); ES-MS m/z 581 (M+1). Anal. Calcd. for C₃₂H₃₆N₈O₃.3.17H₂O.0.76CH₂Cl₂: C, 56.03; H, 6.30; N, 15.96. Found: C, 55.99; H, 6.23; N, 16.08.

### EXAMPLE 150

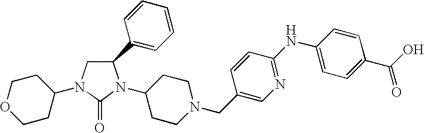

### COMPOUND 150: 4-(5-{4-[(R)-2-Oxo-5-phenyl-3-(tetrahydro-pyran-4-yl)-imidazolidin-1-yl]-piperidin-1-ylmethyl}-pyridin-2-ylamino)-benzoic acid

Following general procedure A, (R)-4-phenyl-3-piperidin-4-yl-1-(tetrahydro-pyran-4-yl)-imidazolidin-2-one (228 mg, 0.692 mmol) and 6-chloro-pyridine-3-carbaldehyde (98 mg, 0.69 mmol) afforded (R)-3-[1-(6-chloro-pyridin-3-ylmethyl)-piperidin-4-yl]-4-phenyl-1-(tetrahydro-pyran-4-yl)-imidazolidin-2-one as a yellow amorphous solid (215 mg, 68%).

A mixture of the above chloride (215 mg, 0.473 mmol), 4-aminobenzonitrile (167 mg, 1.41 mmol), Pd₂(dba)₃ (43 mg, 0.047 mmol), DPPF (52 mg, 0.094 mmol) and Cs₂CO₃ (231 mg, 0.709 mmol) in degassed dioxane (1 mL) was heated to 120° C. for 15 hours under Ar. Standard work-up and purification afforded the nitrile (119 mg). Following general procedure I, the above nitrile afforded COMPOUND 150 as a yellow solid (64 mg, 24% over 2 steps). ¹H NMR (CD₃OD) δ 1.52-1.81 (m, 7H), 2.19 (ddd, 1H, J=25.5, 12.9, 3.9 Hz), 2.44-2.56 (m, 2H), 3.08-3.16 (m, 2H), 3.21 (d, 1H, J=11.7 Hz), 3.42-3.60 (m, 3H), 3.74-3.82 (m, 3H), 3.87-3.99 (m, 3H), 4.72 (dd, 1H, J=9.3, 7.2 Hz), 6.87 (d, 1H, J=8.7 Hz), 7.31-7.39 (m, 5H), 7.57 (dd, 1H, J=8.4, 2.1 Hz), 7.68 (d, 2H, J=8.4 Hz), 7.91 (d, 2H, J=8.7 Hz), 8.13 (d, 1H, J=1.8 Hz); ¹³C NMR (CD₃OD) δ 27.26, 28.33, 29.83, 30.19, 49.49, 50.81, 51.78, 52.04, 56.93, 57.89, 67.19, 67.28, 111.44, 117.50, 118.71, 125.32, 127.08, 128.62, 129.19, 130.90, 140.07, 142.23, 145.43, 149.88, 156.37, 160.69, 170.70; ES-MS m/z 556 (M+1). Anal. Calcd. for C₃₂H₃₇N₅O₄.0.7CH₂Cl₂: C, 63.85; H, 6.29; N, 11.39. Found: C, 63.66; H, 6.50; N, 11.31.

### EXAMPLE 151

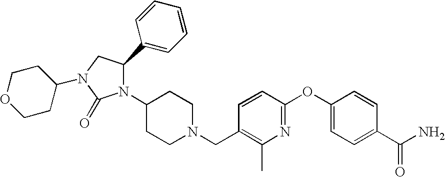

### COMPOUND 151: 4-(6-Methyl-5-{4-[(R)-2-oxo-5-phenyl-3-(tetrahydro-pyran-4-yl)-imidazolidin-1-yl]-piperidine-1-ylmethyl}-pyridin-2-yloxy)-benzamide

Following general procedure A: (R)-4-phenyl-3-piperidin-4-yl-oxazolidin-2-one (63 mg, 0.19 mmol) and 4-(5-formyl-6-methyl-pyridin-2-ylsulfanyl)-benzonitrile (55 mg, 0.23 mmol) were combined in CH₂Cl₂ (1.5 mL) and treated with sodium triacetoxyborohydride (65 mg, 0.30 mmol) at room temperature for 16 h. After standard work-up, the crude material was purified by flash column chromatography on silica gel (50:1:0.1, CH₂Cl₂/MeOH/NH₄OH) to afford 4-(6-methyl-5-{4-[(R)-2-oxo-5-phenyl-3-(tetrahydro-pyran-4-yl)-imidazolidin-1-yl]-piperidine-1-ylmethyl}-pyridin-2-yloxy)-benzonitrile as a white solid (56 mg, 53%).

To a solution of the above nitrile (53 mg, 96 μmol) in TFA (2 mL) was added c. H₂SO₄ (4 drops). The reaction was heated to 95° C. for 16 h, and then concentrated to dryness under reduced pressure. The crude residue was then purified by column chromatography on silica gel (25:1:0.1, CH₂Cl₂/MeOH/NH₄OH) to afford COMPOUND 151 as a white solid (49 mg, 89%). ¹H NMR (CDCl₃) δ 1.22 (dq, 1H, J=12.0, 3.6 Hz), 1.42 (d, 1H, J=12.0 Hz), 1.67 (m, 5H), 1.92 (m, 2H), 2.04 (m, 1H), 2.38 (s, 3H), 2.64 (d, 1H, J=10.5 Hz), 2.83 (d, 1H, J=9.3 Hz), 3.07 (m, 1H), 3.32 (s, 2H), 3.46 (m, 2H), 3.61 (m, 1H), 3.63 (t, 1H, J=9.0 Hz), 4.02 (m, 3H), 4.59 (m, 1H), 6.63 (d, 1H, J=8.1 Hz), 7.14 (d, 2H, J=8.7 Hz), 7.34 (s, 5H), 7.50 (d, 1H, J=8.1 Hz), 7.82 (d, 2H, J=8.1 Hz); ES-MS m/z 570 (M+H).

### EXAMPLE 152

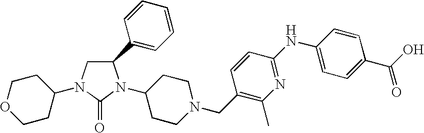

### COMPOUND 152: 4-(6-Methyl-5-{4-[(R)-2-oxo-5-phenyl-3-(tetrahydro-pyran-4-yl)-imidazolidin-1-yl]-piperidin-1-ylmethyl}-pyridin-2-ylamino)-benzoic acid

COMPOUND 152 was prepared using the same chemistry as for COMPOUND 150 except that 6-bromo-2-methyl-pyridine-3-carbaldehyde was used in lieu of 6-chloro-pyridine-3-carbaldehyde. COMPOUND 152 was isolated as a yellow foam. ¹H NMR (CD₃OD) δ 1.51-1.78 (m, 7H), 2.14-2.26 (m, 1H), 2.47-2.59 (m, 5H), 3.07-3.15 (m, 2H), 3.21 (br d, 1H, J=12.0 Hz), 3.38-3.61 (m, 3H), 3.61-3.79 (m, 3H), 3.88-3.99 (m, 3H), 4.73 (dd, 1H, J=9.3, 6.9 Hz), 6.72 (d, 1H, J=8.4 Hz), 7.29-7.38 (m, 5H), 7.48 (d, 1H, J=8.7 Hz), 7.69 (d, 2H, J=8.7 Hz), 7.91 (d, 2H, J=8.7 Hz); ¹³C NMR (CD₃OD) 622.82, 28.86, 29.77, 31.21, 31.55, 50.84, 52.42, 53.59, 53.70, 55.23, 58.33, 58.98, 68.57, 68.66, 110.31, 118.58, 128.44, 129.96, 130.54, 132.24, 142.41, 143.70, 147.05, 156.78, 158.33, 162.12; ES-MS m/z 570 (M+1). Anal. Calcd. for C₃₃H₃₉N₅O₄.1.0CH₂Cl₂.1.8H₂O: C, 59.44; H, 6.54; N, 10.19. Found: C, 59.46; H, 6.57; N, 10.15.

### EXAMPLE 153

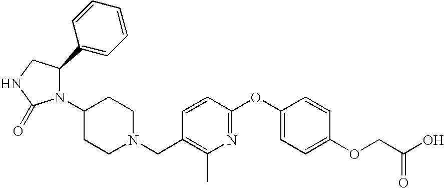

### COMPOUND 153: (4-{6-Methyl-5-[4-((R)-2-oxo-5-phenyl-imidazolidin-1-yl)-piperidin-1-ylmethyl]-pyridin-2-yloxy}-phenoxy)-acetic acid

To a solution of (R)-1-{1-[6-(4-hydroxy-phenoxy)-2-methyl-pyridin-3-ylmethyl]-piperidin-4-yl}-5-phenyl-imidazolidin-2-one (COMPOUND 159) (100.0 mg, 0.218 mmol) in dry THF (1.5 mL) was added NaH (8.7 mg, 0.218 mmol). The solution was stirred at rt for 10 min then t-butyl-bromoacetate (39.4 μL, 0.262 mmol) was added. After stirring at rt for 4 h, the mixture was diluted with NaHCO₃ (30 mL) and extracted with CH₂Cl₂ (3×30 mL). The extracts were dried over Na₂SO₄ and concentrated in vacuo to give the crude product. This was purified by column chromatography on silica gel, eluted with EtOAc then 3% MeOH in CH₂Cl₂ to give (4-{6-methyl-5-[4-((R)-2-oxo-5-phenyl-imidazolidin-1-yl)-piperidin-1-ylmethyl]-pyridin-2-yloxy}-phenoxy)-acetic acid tert-butyl ester (44.4 mg, 36%).

The above product (44.4 mg, 0.078 mmol) was treated with TFA (0.3 mL) in CH₂Cl₂ (0.5 mL) at rt for 5.5 h to give COMPOUND 153 as a white powder (45.3 mg, 100%). ¹H NMR (CD₃OD) δ 1.75-2.00 (m, 3H), 2.35-2.43 (m, 1H), 2.44 (s, 3H), 3.00-3.11 (m, 2H), 3.20 (dd, 1H, J=9.3, 7.5 Hz), 3.40-3.50 (m, 2H), 3.51-3.70 (m, 1H), 3.77 (t, 1H, J=9.3 Hz), 4.24 (s, 2H), 4.66 (s, 2H), 4.82 (dd, 1H, J=9.3, 7.5 Hz), 6.69 (d, 1H, J=8.7 Hz), 6.96-7.05 (m, 4H), 7.31-7.44 (m, 5H), 7.73 (d, 1H, J=8.7 Hz); ¹³C NMR (CD₃OD) δ 22.5, 28.0, 28.7, 50.9, 53.4, 53.5, 57.7, 61.0, 66.9, 109.7, 117.2, 119.5, 123.7, 128.5, 130.1, 130.6, 143.4, 145.6, 149.5, 157.2, 159.8, 164.8, 166.0, 173.1; ES-MS m/z 517 (M+H). Anal. Calcd. for C₂₉H₃₂N₄O₅.1.5CH₂Cl₂.0.1MeOH: C, 56.79; H, 5.51; N, 8.66. Found: C, 56.91; H, 5.35; N, 8.35.

### EXAMPLE 154

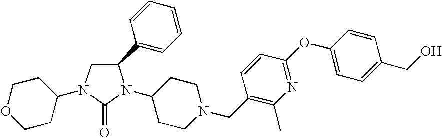

### COMPOUND 154: (R)-3-{1-[6-(4-Hydroxymethyl-phenoxy)-2-methyl-pyridin-3-ylmethyl]-piperidin-4-yl}-4-phenyl-1-(tetrahydro-pyran-4-yl)-imidazolidin-2-one

To a solution of 4-(6-methyl-5-{4-[(R)-2-oxo-5-phenyl-3-(tetrahydro-pyran-4-yl)-imidazolidin-1-yl]-piperidin-1-ylmethyl}-pyridin-2-yloxy)-benzoic acid methyl ester (see EXAMPLE 74) (641 mg, 1.10 mmol) in THF (7 mL) at 0° C. was added LAH (1.0M in THF, 2.2 mL, 2.2 mmol) and the mixture stirred at 0° C. for 50 minutes. H₂O (0.08 mL) was added followed by 15% NaOH (0.08 mL) and H₂O (0.24 mL) and the mixture stirred for 1 hour at room temperature. Standard work-up and purification afforded COMPOUND 154 as a colourless foam (585 mg, 96%). ¹H NMR (CDCl₃) δ 1.17 (ddd, 1H, J=24.3, 12.0, 3.6 Hz), 1.38-1.43 (m, 1H), 1.64-1.74 (m, 5H), 1.82-2.05 (m, 4H), 2.38 (s, 3H), 2.62-2.66 (m, 1H), 2.80-2.83 (m, 1H), 3.06 (dd, 1H, J=8.4, 6.9 Hz), 3.25-3.35 (m, 2H), 3.44-3.52 (m, 2H), 3.59-3.68 (m, 2H), 3.97-4.05 (m, 3H), 4.59 (dd, 1H, J=9.3, 6.9 Hz), 4.68 (d, 2H, J=4.8 Hz), 6.50 (d, 1H, J=8.1 Hz), 7.07-7.10 (m, 2H), 7.30-7.37 (m, 7H), 7.43 (d, 1H, J=8.4 Hz); ES-MS m/z 557 (M+1).

### EXAMPLE 155

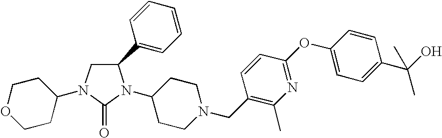

### COMPOUND 155: (R)-3-(1-{6-[4-(1-Hydroxy-1-methyl-ethyl)-phenoxy]-2-methyl-pyridin-3-ylmethyl}-piperidin-4-yl)-4-phenyl-1-(tetrahydro-pyran-4-yl)-imidazolidin-2-one

To a solution of 4-(6-methyl-5-{4-[(R)-2-oxo-5-phenyl-3-(tetrahydro-pyran-4-yl)-imidazolidin-1-yl]-piperidin-1-ylmethyl}-pyridin-2-yloxy)-benzoic acid methyl ester (see EXAMPLE 74) (85.5 mg, 0.146 mmol) in THF (1 mL) cooled to 0° C. was added MeMgBr (3M in Et₂O, 0.2 mL, 0.6 mmol) and the mixture was stirred at 0° C. for 2 hours. Standard work-up and purification afforded COMPOUND 155 as a white solid (32 mg, 38%). ¹H NMR (CDCl₃) δ 1.14-1.25 (m, 1H), 1.38-1.43 (m, 1H), 1.59-1.70 (m, 11H), 1.83-2.01 (m, 4H), 2.39 (s, 3H), 2.62-2.66 (m, 1H), 2.80-2.83 (m, 1H), 3.06 (dd, 1H, J=8.4, 6.9 Hz), 3.29 (s, 2H), 3.43-3.52 (m, 2H), 3.61-3.67 (m, 2H), 3.97-4.04 (m, 3H), 4.59 (dd, 1H, J=8.4, 6.6 Hz), 6.49 (d, 1H, J=8.4 Hz), 7.06 (d, 2H, J=8.4 Hz), 7.31-7.33 (m, 5H), 7.41-7.49 (m, 3H); ¹³C NMR (CDCl₃) δ 21.89, 29.13, 29.82, 30.18, 30.85, 31.82, 48.39, 48.70, 52.22, 53.10, 53.25, 53.43, 56.15, 58.93, 67.21, 67.30, 72.29, 107.24, 120.16, 125.81, 126.70, 126.81, 128.24, 128.84, 140.79, 142.67, 144.98, 153.39, 156.58, 160.05, 161.84; ES-MS m/z 585 (M+1). Anal. Calcd. for C₃₅H₄₄N₄O₄.0.27CH₄O.0.18CH₂Cl₂: C, 69.98; H, 7.53; N, 9.21. Found: C, 69.99; H, 7.50; N, 9.15.

### EXAMPLE 156

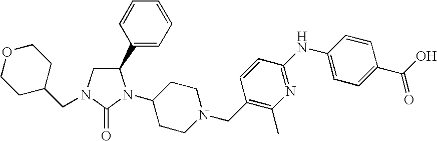

### COMPOUND 156: 4-(6-Methyl-5-{4-[(R)-2-oxo-5-phenyl-3-(tetrahydro-pyran-4-ylmethyl)-imidazolidin-1-yl-piperidin-1-ylmethyl}-pyridin-2-ylamino)-benzoic acid

COMPOUND 156 was prepared using the same chemistry as COMPOUND 152 except that (R)-4-phenyl-3-piperidin-4-yl-1-(tetrahydro-pyran-4-ylmethyl)-imidazolidin-2-one was used in lieu of (R)-4-phenyl-3-piperidin-4-yl-1-(tetrahydro-pyran-4-yl)-imidazolidin-2-one. COMPOUND 156 was isolated as a yellow foam. ¹H NMR (CD₃OD) δ 1.23-1.41 (m, 2H), 1.62-1.66 (m, 2H), 1.83-1.97 (m, 4H), 2.35-2.48 (m, 1H), 2.57 (s, 3H), 3.02-3.18 (m, 4H), 3.22 (dd, 1H, J=8.7, 7.2 Hz), 3.38-3.55 (m, 4H), 3.61-3.69 (m, 1H), 3.84 (t, 1H, J=9.3 Hz), 3.95-3.99 (m, 2H), 4.24 (s, 2H), 4.80 (dd, 1H, J=9.0, 7.2 Hz), 6.81 (d, 1H, J=8.7 Hz), 7.37-7.45 (m, 5H), 7.61 (d, 1H, J=8.7 Hz), 7.83 (d, 2H, J=8.7 Hz), 7.96 (d, 2H, J=8.7 Hz); ¹³C NMR (CD₃OD) δ 23.66, 28.72, 29.19, 32.86, 35.87, 51.70, 52.15, 53.90, 54.02, 55.42, 59.27, 69.64, 111.38, 116.19, 119.48, 124.88, 129.20, 130.75, 131.30, 132.98, 143.45, 143.93, 148.25, 158.11, 159.74, 163.69, 171.11; ES-MS m/z 584 (M+1). Anal. Calcd. for C₃₄H₄₁N₅O₄.0.7CH₂Cl₂.1.6H₂O: C, 62.02; H, 6.84; N, 10.42. Found: C, 61.85; H, 6.81; N, 10.46.

### EXAMPLE 157

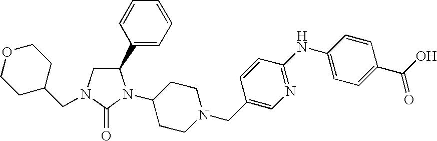

### COMPOUND 157: 4-(5-{4-[(R)-2-Oxo-5-phenyl-3-(tetrahydro-pyran-4-ylmethyl)-imidazolidin-1-yl]-piperidin-1-ylmethyl}-pyridin-2-ylamino)-benzoic acid

COMPOUND 157 was prepared using the same chemistry as COMPOUND 150 except that (R)-4-phenyl-3-piperidin-4-yl-1-(tetrahydro-pyran-4-ylmethyl)-imidazolidin-2-one and 6-bromo-pyridine-3-carbaldehyde were used in lieu of (R)-4-phenyl-3-piperidin-4-yl-1-(tetrahydro-pyran-4-yl)-imidazolidin-2-one and 6-chloro-pyridine-3-carbaldehyde, respectively. COMPOUND 157 was isolated as a brown foam. ¹H NMR (CDCl₃) δ 1.25-1.41 (m, 2H), 1.62-1.66 (m, 2H), 1.83-1.97 (m, 4H), 2.35-2.47 (m, 1H), 2.96-3.10 (m, 2H), 3.13 (dd, 1H, J=7.2, 2.7 Hz), 3.22 (dd, 1H, J=8.7, 7.5 Hz), 3.38-3.65 (m, 5H), 3.84 (t, 1H, J=9.3 Hz), 3.95-3.99 (m, 2H), 4.19 (s, 2H), 4.80 (dd, 1H, J=9.3, 7.5 Hz), 6.96 (d, 1H, J=8.4 Hz), 7.38-7.45 (m, 5H), 7.68 (dd, 1H, J=8.7, 2.1 Hz), 7.79 (d, 2H, J=8.7 Hz), 7.97 (d, 2H, J=8.7 Hz), 8.27 (d, 1H, J=2.1 Hz); ES-MS m/z 570 (M+1).

### EXAMPLE 158

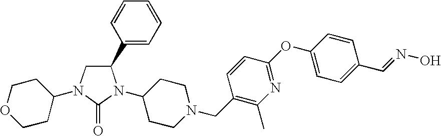

### COMPOUND 158: 4-(6-Methyl-5-{4-[(R)-2-oxo-5-phenyl-3-(tetrahydro-pyran-4-yl)-imidazolidin-1-yl]-piperidin-1-ylmethyl}-pyridin-2-yloxy)-benzaldehyde oxime

To a solution of 4-(6-methyl-5-{4-[(R)-2-oxo-5-phenyl-3-(tetrahydro-pyran-4-yl)-imidazolidin-1-yl]-piperidin-1-ylmethyl}-pyridin-2-yloxy)-benzoic acid methyl ester (see EXAMPLE 74) (549 mg, 0.939 mmol) in THF (9 ml) at 0° C. was added LAH (1.0M in THF, 1.9 mL, 1.9 mmol). The mixture was stirred at 0° C. for 30 minutes. H₂O (0.07 mL) was added followed by 15% NaOH (0.07 mL) and H₂O (0.2 mL) and the mixture was stirred at room temperature for 10 minutes. Standard work-up afforded the alcohol (497 mg, 95%). To the alcohol (497 mg, 0.893 mmol) in CH₂Cl₂ (9 mL) was added MnO₂ (85%, 1.4 g, 14 mmol) and the mixture stirred at room temperature for 20 hours. Standard work-up afforded 4-(6-methyl-5-{4-[(R)-2-oxo-5-phenyl-3-(tetrahydro-pyran-4-yl)-imidazolidin-1-yl]-piperidin-1-ylmethyl}-pyridin-2-yloxy)-benzaldehyde as a green foam (457 mg, 92%).

A solution of the above aldehyde (78 mg, 0.14 mmol) and hydroxylamine hydrochloride (20 mg, 0.29 mmol) in MeOH (2.0 mL) was stirred at room temperature for 1 hour. Aqueous work-up and purification afforded COMPOUND 158 as a colourless foam (48 mg, 60%). ¹H NMR (CDCl₃) δ 1.13-1.25 (m, 1H), 1.40-1.44 (m, 1H), 1.57-1.72 (m, 5H), 1.84-2.08 (m, 3H), 2.39 (s, 3H), 2.64-2.68 (m, 1H), 2.82-2.86 (m, 1H), 3.07 (dd, 1H, J=8.4, 6.6 Hz), 3.27-3.37 (m, 2H), 3.44-3.53 (m, 2H), 3.62-3.71 (m, 2H), 3.97-4.11 (m, 3H), 4.59 (dd, 1H, J=9.0, 6.6 Hz), 6.57 (d, 1H, J=8.4 Hz), 7.09 (d, 2H, J=8.7 Hz), 7.28-7.37 (m, 5H), 7.48 (d, 1H, J=8.1 Hz), 7.54-7.57 (m, 2H), 7.90 (br s, 1H), 8.11 (s, 1H); ¹³C NMR (CDCl₃) δ 21.88, 28.87, 29.82, 30.13, 30.68, 48.41, 48.69, 52.06, 53.11, 53.24, 56.03, 58.74, 67.20, 67.29, 108.04, 120.52, 126.67, 127.01, 128.22, 128.50, 128.84, 141.27, 142.64, 148.88, 156.01, 156.62, 160.13, 161.23; ES-MS m/z 570 (M+1). Anal. Calcd. for C₃₃H₃₉N₅O₄.0.4CH₂Cl₂: C, 66.45; H, 6.65; N, 11.60. Found: C, 66.46; H, 6.65; N, 11.46.

### EXAMPLE 159

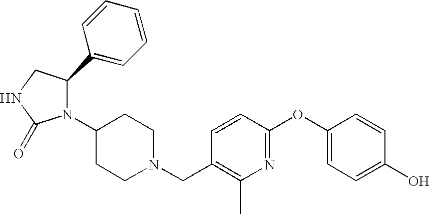

### COMPOUND 159: (R)-1-{1-[6-(4-Hydroxy-phenoxy)-2-methyl-pyridin-3-ylmethyl]-piperidin-4-yl}-5-phenyl-imidazolidin-2-one

To a solution of (R)-1-{1-[6-(4-methoxy-phenoxy)-2-methyl-pyridin-3-ylmethyl]-piperidin-4-yl}-5-phenyl-imidazolidin-2-one (COMPOUND 72) (357.6 mg, 0.758 mmol) in CH₂Cl₂ (16.3 mL) was added a solution of BBr₃ (1 M in CH₂Cl₂, 3.788 mL, 3.788 mmol) at 0° C. The mixture was stirred at rt for 18.5 h. MeOH (5 mL) was added to the mixture. Standard work-up and purification afforded COMPOUND 159 as a yellow powder (204.9 mg, 75%). ¹H NMR (CDCl₃) δ 1.05-1.18 (m, 1H), 1.39 (d, 1H, J=12.0 Hz), 1.69 (d, 1H, J=11.7 Hz), 1.89-2.04 (m, 2H), 2.12 (t, 1H, J=10.8 Hz), 2.43 (s, 3H), 2.69 (d, 1H, J=10.8 Hz), 2.94 (d, 1H, J=10.5 Hz), 3.20 (t, 1H, J=10.5 Hz), 3.30 (d, 1H, J=13.2 Hz), 3.40 (d, 1H, J=13.2 Hz), 3.66-3.76 (m, 1H), 3.73 (t, 1H, J=9.0 Hz), 4.67 (dd, 1H, J=9.0, 6.6 Hz), 4.80 (br s, 1H), 6.22 (d, 1H, J=8.4 Hz), 6.60 (d, 2H, J=8.7 Hz), 6.85 (d, 2H, J=8.7 Hz), 7.16-7.25 (m, 5H), 7.41 (d, 1H, J=8.4 Hz); ¹³C NMR (CDCl₃) δ 22.3, 29.0, 31.0, 48.6, 51.9, 53.0, 53.7, 58.8, 106.3, 116.9, 122.5, 125.5, 127.1, 128.6, 129.1, 142.1, 142.5, 146.9, 154.3, 157.0, 163.1, 163.7; ES-MS m/z 459 (M+H). Anal. Calcd. for C₂₇H₃₀N₄O₃.0.5CH₂Cl₂.0.6EtOAc: C, 64.84; H, 6.51; N, 10.12. Found: C, 65.16; H, 6.35; N, 10.11.

### EXAMPLE 160

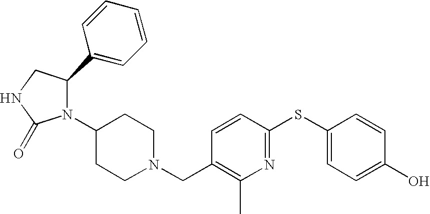

### COMPOUND 160: (R)-1-{1-[6-(4-Hydroxy-phenylsulfanyl)-2-methyl-pyridin-3-ylmethyl]-piperidin-4-yl}-5-phenyl-imidazolidin-2-one

To a solution of (R)-1-{1-[6-(4-methoxy-phenylsulfanyl)-2-methyl-pyridin-3-ylmethyl]-piperidin-4-yl}-5-phenyl-imidazolidin-2-one (COMPOUND 64) (250.0 mg, 0.512 mmol) in CH₂Cl₂ (11.0 mL) was added a solution of BBr₃ in CH₂Cl₂ (1 M, 2.56 mL, 2.56 mmol) at 0° C. The mixture was stirred at rt for 17 h. MeOH (5 mL) was added to the mixture. The mixture was concentrated in vacuo. Standard work-up and purification gave COMPOUND 160 as a white powder (176 mg, 72%). ¹H NMR (CDCl₃) δ 1.08-1.22 (m, 1H), 1.39 (d, 1H, J=12.8 Hz), 1.67 (d, 1H, J=11.7 Hz), 1.80-2.01 (m, 2H), 2.08 (t, 1H, J=10.8 Hz), 2.46 (s, 3H), 2.68 (d, 1H, J=11.1 Hz), 2.89 (d, 1H, J=9.9 Hz), 3.19 (t, 1H, J=7.5 Hz), 3.29 (d, 1H, J=13.5 Hz), 3.35 (d, 1H, J=13.5 Hz), 3.60-3.70 (m, 1H), 3.73 (t, 1H, J=9.0 Hz), 4.63 (dd, 1H, J=9.0, 6.6 Hz), 4.68 (s, 1H), 6.39 (d, 1H, J=8.1 Hz), 6.71 (d, 2H, J=8.7 Hz), 7.22 (br s, 5H), 7.27 (d, 1H, J=8.1 Hz), 7.36 (d, 2H, J=8.7 Hz); ¹³C NMR (CDCl₃) δ 20.8, 27.7, 29.5, 47.2, 50.6, 51.9, 52.1, 57.5, 57.8, 116.2, 116.3, 118.1, 125.7, 125.8, 127.3, 127.8, 136.6, 137.6, 141.0, 156.4, 157.6, 160.5, 161.8; ES-MS m/z 475 (M+H). Anal. Calcd. for C₂₇H₃₀N₄O₂S.0.2CH₂Cl₂: C, 66.46; H, 6.23; N, 11.40. Found: C, 66.79; H, 6.48; N, 11.09.

### EXAMPLE 161

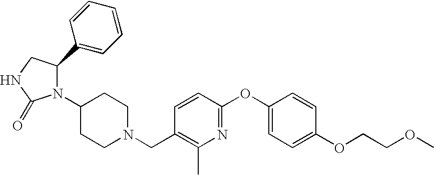

### COMPOUND 161: (R)-1-(1-{6-[4-(2-Methoxy-ethoxy)-phenoxy]-2-methyl-pyridin-3-ylmethyl}-piperidin-4-yl)-5-phenyl-imidazolidin-2-one

To a solution of (R)-1-{1-[6-(4-hydroxy-phenoxy)-2-methyl-pyridin-3-ylmethyl]-piperidin-4-yl}-5-phenyl-imidazolidin-2-one (COMPOUND 159) (100.0 mg, 0.218 mmol) in dry THF (1.5 mL) was added NaH (10.5 mg, 0.262 mmol). After stirring at rt for 10 min, 2-bromo-ethyl-methyl ether (24.6 μL, 0.262 mmol) was added to the mixture. The mixture was heated at 50° C. for 23 h. Aqueous work-up and purification gave COMPOUND 161 as a white powder (44.1 mg, 39%). ¹H NMR (CDCl₃) δ 1.09-1.25 (m, 1H), 1.40 (d, 1H, J=12.0 Hz), 1.66 (d, 1H, J=10.8 Hz), 1.85-1.92 (m, 2H), 2.00 (t, 1H, J=10.8 Hz), 2.37 (s, 3H), 2.62 (d, 1H, J=10.5 Hz), 2.81 (d, 1H, J=10.2 Hz), 3.21 (t, 1H, J=7.5 Hz), 3.28 (s, 2H), 3.45 (s, 3H), 3.69 (m, 1H), 3.71-3.76 (m, 3H), 4.11 (t, 2H, J=4.8 Hz), 4.71 (dd, 1H, J=8.7, 6.6 Hz), 4.78 (br s, 1H), 6.40 (d, 1H, J=8.1 Hz), 6.91 (d, 2H, J=8.7 Hz), 7.02 (d, 2H, J=8.7 Hz), 7.30-7.40 (m, 6H); ¹³C NMR (CDCl₃) δ 22.3, 29.6, 31.3, 48.6, 52.2, 53.4, 53.6, 58.9, 59.3, 59.6, 68.1, 71.5, 106.8, 115.9, 122.3, 126.7, 127.2, 128.6, 129.2, 141.2, 142.9, 148.6, 155.9, 156.9, 162.9, 163.0; ES-MS m/z 517 (M+H). Anal. Calcd. for C₃₀H₃₆N₄O₄.0.1CH₂Cl₂.0.1H₂O: C, 68.61; H, 6.96; N, 10.63. Found: C, 68.43; H, 6.93; N, 10.57.

### EXAMPLE 162

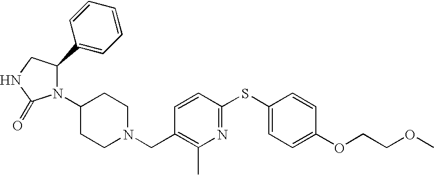

### COMPOUND 162: (R)-1-(1-{6-[4-(2-Methoxy-ethoxy)-phenylsulfanyl]-2-methyl-pyridin-3-ylmethyl}-piperidin-4-yl)-5-phenyl-imidazolidin-2-one

To a solution of (R)-1-{1-[6-(4-hydroxy-phenylsulfanyl)-2-methyl-pyridin-3-ylmethyl]-piperidin-4-yl}-5-phenyl-imidazolidin-2-one (COMPOUND 160) (63.0 mg, 0.133 mmol) in DMF (1.0 mL) was added NaH (6.4 mg, 0.160 mmol). The mixture was stirred at rt for 10 min then 2-bromo-ethyl-methyl ether (15 μL, 0.160 mmol) was added. The mixture was stirred at 60° C. for 4 h. The mixture was concentrated in vacuo to remove DMF. Aqueous work-up and purification gave COMPOUND 162 as a white powder (42.6 mg, 60%). ¹H NMR (CDCl₃) δ 1.04-1.15 (m, 1H), 1.36 (d, 1H, J=12.0 Hz), 1.62 (d, 1H, J=10.8 Hz), 1.84 (t, 2H, J=11.4 Hz), 1.97 (t, 1H, J=11.4 Hz), 2.41 (s, 3H), 2.57 (d, 1H, J=10.8 Hz), 2.76 (d, 1H, J=10.2 Hz), 3.18-3.27 (m, 3H), 3.45 (s, 3H), 3.63 (t, 1H, J=12.0 Hz), 3.70-3.83 (m, 3H), 4.14 (m, 2H), 4.69 (t, 1H, J=7.6 Hz), 5.15 (s, 1H), 6.42 (d, 1H, J=8.1 Hz), 6.95 (d, 2H, J=8.1 Hz), 7.17 (d, 1H, J=8.1 Hz), 7.27-3.33 (m, 5H), 7.48 (d, 2H, J=8.1 Hz); ¹³C NMR (CDCl₃) δ 22.4, 29.5, 31.4, 48.6, 52.2, 53.4, 53.6, 58.9, 59.7, 67.8, 71.3, 116.2, 117.7, 122.2, 127.2, 128.2, 128.6, 129.2, 137.6, 138.3, 142.9, 158.1, 160.2, 160.5, 163.0; ES-MS m/z 533 (M+H). Anal. Calcd. for C₃₀H₃₆N₄O₃S.0.3CH₂Cl₂: C, 65.20; H, 6.61; N, 10.04. Found: C, 65.24; H, 6.65; N, 9.96.

### EXAMPLE 163

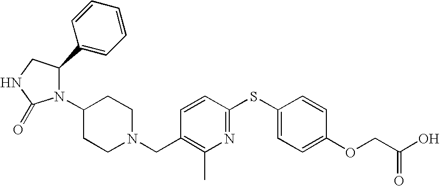

### COMPOUND 163: (4-{6-Methyl-5-[4-((R)-2-oxo-5-phenyl-imidazolidin-1-yl)-piperidin-1-ylmethyl]-pyridin-2-ylsulfanyl}-phenoxy)-acetic acid

To a solution of (R)-1-{1-[6-(4-hydroxy-phenylsulfanyl)-2-methyl-pyridin-3-ylmethyl]-piperidin-4-yl}-5-phenyl-imidazolidin-2-one (COMPOUND 160) (70.0 mg, 0.148 mmol) in dry THF (1.5 mL) was added NaH (7.09 mg, 0.177 mmol). The solution was stirred at rt for 10 min then t-butyl-bromoacetate (26.1 μL, 0.177 mmol) was added. The mixture was stirred at rt for 3 days. Standard work-up and purification gave (4-{6-methyl-5-[4-((R)-2-oxo-5-phenyl-imidazolidin-1-yl)-piperidin-1-ylmethyl]-pyridin-2-ylsulfanyl}-phenoxy)-acetic acid tert-butyl ester (53.2 mg, 61%).

The above product (53.2 mg, 0.091 mmol) was treated with TFA (0.3 mL) in CH₂Cl₂ (0.5 mL) at rt for 4.5 h to give COMPOUND 163 as a yellow powder (47.9 mg, 100%). ¹H NMR (CD₃OD) δ 1.31 (m, 1H), 1.82 (m, 3H), 2.39 (m, 1H), 2.51 (s, 3H), 3.05 (m, 2H), 3.20 (m, 1H), 3.64 (m, 1H), 3.79 (m, 1H), 4.23 (s, 2H), 4.74 (s, 2H), 4.82 (m, 1H), 5.50 (m, 1H), 6.64 (br s, 1H), 7.07 (br s, 2H), 7.30-7.40 (m, 5H), 7.48-7.54 (m, 3H); ¹³C NMR (CD₃OD) δ 21.2, 26.5, 27.2, 29.8, 49.4, 52.0, 52.2, 53.9, 56.5, 59.6, 116.4, 118.1, 119.6, 121.0, 127.1, 128.7, 129.2, 137.5, 141.1, 142.0, 158.9, 160.0, 163.3, 164.8, 171.5; ES-MS m/z 533 (M+H). Anal. Calcd. for C₂₉H₃₂N₄O₄S.1.5CH₂Cl₂: C, 55.50; H, 5.34; N, 8.49. Found: C, 55.52; H, 5.28; N, 8.26.

Examples 164 to 173 were prepared following the scheme illustrated below. RCH₂Br is as defined in the table and X is as defined in the individual examples.

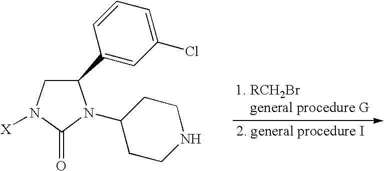

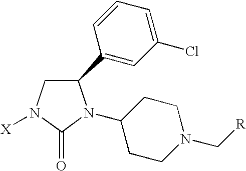

| Example | RCH₂Br |
|---|---|
| 164 | 4-(5-bromomethyl-pyridin-2-yloxy)-benzonitrile |
| 165 | 4-(5-bromomethyl-pyridin-2-yloxy)-benzonitrile |
| 166 | 4-(5-bromomethyl-pyridin-2-yloxy)-benzonitrile |
| 167 | 4-(5-bromomethyl-pyridin-2-yloxy)-benzonitrile |
| 168 | 4-(5-bromomethyl-pyridin-2-yloxy)-benzonitrile |
| 169 | 4-(5-bromomethyl-pyridin-2-yloxy)-benzonitrile |
| 170 | 4-(5-bromomethyl-pyridin-2-yloxy)-benzonitrile |
| 171 | 4-(5-bromomethyl-pyridin-2-yloxy)-benzonitrile |
| 172 | 4-(5-bromomethyl-pyridin-2-yloxy)-benzonitrile |
| 173 | 4-(5-bromomethyl-pyridin-2-yloxy)-benzonitrile |

### EXAMPLE 164

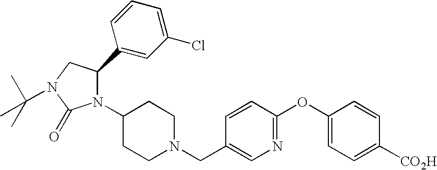

### COMPOUND 164: 4-(5-{4-[(R)-3-tert-Butyl-5-(3-chloro-phenyl)-2-oxo-imidazolidin-1-yl]piperidin-1-ylmethyl}-pyridin-2-yloxy)-benzoic acid

To a solution of NaCN (2.72 g, 55.5 mmol), NH₄Cl (2.97 g, 55.5 mmol) in ammonium hydroxide (28%, 28 mL) was added a solution of 3-chlorobenzaldehyde (3.90 g, 27.7 mmol) in methanol (14 mL). The mixture was stirred at rt for 2 h. The solvents were removed by vacuum vaporation. To the residue was added 6 N HCl (20 mL) and the mixture was heated at reflux for 90 min. The solvents were removed by vacuum evaporation. The residue was dissolved in THF (20 mL) and aqueous NaOH (5.5 N, 21 mL). Boc₂O (6.04 g, 27.7 mmol) was added and the mixture was stirred at rt for 2 h. The solution was neutralized with 3 N HCl to pH=3˜4 and then extracted with CHCl₃ (3×100 mL). The extracts were dried over Na₂SO₄ and the solvents were evaporated to give the crude product. This was purified by silica gel column chromotography, eluted with 3-10% MeOH in dichloromethane to give (+/−)-tert-butoxycarbonylamino-(3-chlorophenyl)-acetic acid (4.71 g, 59%).

Following general procedure E, (+/−)-tert-butoxycarbonylamino-(3-chlorophenyl)-acetic acid (4.34 g, 15.2 mmol) was reacted with (R)-1-(4-methoxy-phenyl)-ethylamine (2.30 g, 15.2 mmol) to provide a 1:1 mixture of the title compounds. This was separated by SiO₂ column chromatography (toluene-EtOAc, 9:1 to 3:1) to afford {(3S)-chlorophenyl)-[(1R)-(4-methoxyphenyl)-ethylcarbamoyl]-methyl}-carbamic acid tert-butyl ester (2.20 g, de>98%, 35%) and {(3R)-chlorophenyl)-[(1R)-(4-methoxyphenyl)-ethylcarbamoyl]-methyl}-carbamic acid tert-butyl ester (2.30 g, de>98%, 36%) successively. The 1:1 mixture of the two compounds could also be obtained from either pure compound by treatment with 10 N NaOH in MeOH.

The above {(3R)-chlorophenyl)-[1(R)-(4-methoxyphenyl)-ethylcarbamoyl]-methyl}-carbamic acid tert-butyl ester (2.12 g, 5.07 mmol) was heated in 6 N HCl (8.5 mL) at reflux for 2 h. The solvents were removed by vacuum evaporation. The residue was dissolved in THF (20 mL) and water (10 mL). Boc₂O (2.213 g, 10.15 mmol) and Et₃N (1.77 mL) were added and the mixture was stirred at rt for 3 h. The solution was neutralized with 3 N HCl to pH=3˜4 and then extracted with CHCl₃ (3×30 mL). The extracts were dried over Na₂SO₄ and the solvents were evaporated to give the crude product. This was purified by silica gel column chromatography (3-10% MeOH in CH₂Cl₂) to give (R)-tert-butoxycarbonylamino-(3-chlorophenyl)-acetic acid (1.403 g, 97%, ee=93%). Once crystallization of this from CH₂Cl₂-hexane gave crystal (954 mg, ee>99%).

Following general procedure F, (R)-tert-butoxycarbonylamino-(3-chlorophenyl)-acetic acid (664 mg, 2.33 mmol, ee=99%) was reacted with t-butylamine (221 mg, 3.03 mmol) to provide {tert-butylcarbamoyl-[(3R)-chlorophenyl]-methyl}-carbamic acid tert-butyl ester (602 mg, ee=91%, 76%). This was crystallized from ethyl acetate-hexane to give the product from the mother liquid (540 mg, ee=98%).

Following general procedure C, {tert-butylcarbamoyl-[(3R)-chlorophenyl]-methyl}-carbamic acid tert-butyl ester (525 mg, 1.54 mmol, ee=98%) was treated with TFA (1.5 mL) in dichloromethane (5 mL) to provide 2-amino-N-tert-butyl-2-[(3R)-chlorophenyl]-acetamide (372 mg, 100%).

To a solution of above product (372 mg, 1.54 mmol) in dry THF (5 mL) was added a solution of BH₃-THF complex in THF (1.0 M, 4.63 mL). The mixture was heated at reflux for 3 h. After cooling to rt, methanol (2 mL) was added and the mixture was heated at reflux for 15 min. The solvents were removed by evaporation under reduced pressure. The residue was redissolved in methanol (5 mL) and ethylenediamine (2 mL) was added. The mixture was heated at 60° C. for 15 min. Standard work-up and purification by column chromatography on silica gel (94:4:2, CH₂Cl₂/MeOH/NH₄OH) afforded N²-tert-butyl-1-[(3R)-chlorophenyl]-ethane-1,2-diamine (274 mg, 79%).

Following general procedure A, N²-tert-butyl-1-[(3R)-chlorophenyl]-ethane-1,2-diamine (70.0 mg, 0.31 mmol) was reacted with 1-Boc-piperidone (61.5 mg, 0.31 mmol) to provide 4-{2-tert-butylamino-1-[(3R)-chlorophenyl]-ethylamino}-piperidine-1-carboxylic acid tert-butyl ester (112 mg, 88%).

To a solution of above product (112 mg, 0.273 mmol) and pyridine (55 μL, 0.68 mmol)) in dry dichloromethane (2 mL) at 0° C. was added triphosgene (32.4 mg, 0.109 mmol). The mixture was stirred at rt for 1.5 h. The solvent was removed by evaporation and the residue was purified by column chromatography on silica gel (3:1, hexane/EtOAc) to give 4-{3-tert-butyl-5-[(3R)-chlorophenyl]-2-oxo-imidazolidin-1-yl}-piperidine-1-carboxylic acid tert-butyl ester (76 mg, 64%).

Following general procedure C, the above product (76 mg, 0.18 mmol) was treated with TFA (1 mL) in CH₂Cl₂ (2 mL) to provide 1-tert-butyl-4-[(3R)-chlorophenyl]-3-piperidin-4-yl-imidazolidin-2-one (56 mg, 96%).

COMPOUND 164 was isolated as a pale yellow powder (24.5 mg, 74% over 2 steps). ¹H NMR (CDCl₃) δ 1.36 (s, 9H), 1.56-1.89 (m, 3H), 2.17-2.28 (m, 1H), 2.78-2.89 (m, 2H), 3.14 (t, 1H, J=8.0 Hz), 3.27-3.42 (m, 2H), 3.64 (m, 1H), 3.81 (t, 1H, J=9.0 Hz), 4.11 (s, 2H), 4.63 (t, 1H, J=8.0 Hz), 7.08 (d, 1H, J=8.4 Hz), 7.18 (br s, 2H), 7.31-7.38 (m, 3H), 7.42 (s, 1H), 7.93 (d, 1H, J=6.9 Hz), 8.02 (br s, 2H), 8.18 (s, 1H); ¹³C NMR (CDCl₃) δ 26.75, 26.93, 27.97, 50.25, 50.25, 50.54, 51.96, 52.12, 53.50, 55.62, 56.98, 112.38, 120.65, 122.46, 125.49, 127.18, 128.62, 130.78, 131.73, 134.82, 143.17, 144.73, 150.06, 157.85, 161.27, 164.11; ES-MS m/z 563 (M+H). Anal. Calcd. for C₃₁H₃₅ClN₄O₄.1.1H₂O.1.0CH₂Cl₂: C, 57.55; H, 5.92; N, 8.39. Found: C, 57.40; H, 5.92; N, 8.32.

### EXAMPLE 165

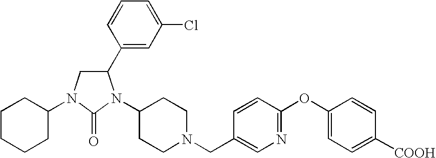

### COMPOUND 165: 4-(5-{4-[5-(3-Chloro-phenyl)-3-cyclohexyl-2-oxo-imidazolidin-1-yl]-piperidin-1-ylmethyl}-pyridin-2-yloxy)-benzoic acid

COMPOUND 165 was isolated as a yellow solid (84 mg, 50% in two steps). ¹H NMR (CDCl₃) δ 0.99-1.07 (m, 1H), 1.32-1.38 (m, 4H), 1.51-1.61 (m, 4H), 1.67-1.82 (m, 2H), 2.15-2.21 (m, 1H), 2.40-2.61 (m, 1H), 3.04-3.31 (m, 4H), 3.65-3.98 (m, 3H), 3.88 (s, 2H), 4.70 (dd, 1H, J=6, 3 Hz), 4.90 (s, 3H), 7.01 (d, 1H, J=8.4 Hz), 7.13 (d, 2H, J=8.4 Hz), 7.27-7.30 (m, 3H), 7.36 (s, 1H), 7.85 (dd, 1H, J=6, 2.4 Hz), 8.02 (d, 2H, J=8.7 Hz), 8.12 (d, 1H, J=2.1 Hz); ¹³C NMR (d-MeOH) δ 29.40, 29.59, 29.67, 31.44, 32.88, 33.87, 34.10, 54.96, 56.01, 56.27, 57.76, 60.06, 61.45, 116.00, 123.85, 124.32, 128.60, 129.29, 130.94, 132.48, 133.47, 134.67, 135.51, 138.74, 146.74, 149.04, 150.15, 153.46, 161.78, 164.61, 167.57, 173.68; ES-MS m/z 589 (M+1). Anal. Calcd. for C₃₃H₃₇N₄O₄Cl.0.57CH₂Cl₂.0.08H₂O: C, 63.12; H, 6.04; N, 8.77. Found: C, 63.14; H, 6.07; N, 8.66.

### EXAMPLE 166

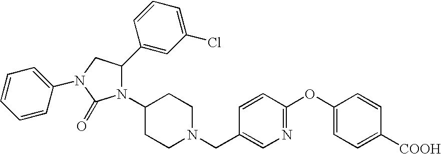

### COMPOUND 166: 4-(5-{4-[5-(3-Chloro-phenyl)-2-oxo-3-phenyl-imidazolidin-1-yl]-piperidin-1-ylmethyl}-pyridin-2-yloxy)-benzoic acid

Following general procedure F, tert-butoxycarbonylamino-(3-chloro-phenyl)-acetic acid (0.342 g, 1.20 mmol), N-methylmorpholine (0.131 g, 1.30 mmol), isobutyl chloroformate (0.178 g, 1.30 mmol) and aniline (0.0279 g, 3.00 mmol) afforded the amide as a white solid (0.31 g).

Following general procedure C, the above substrate afforded a sticky oil (0.23 g). The oil was dissolved in dry THF (6 mL), and BH₃-THF (1.0 M) (4.0 mL, 4.0 mmol) was added. After stirring at 65° C. overnight, the mixture was cooled to room temperature and methanol (1 mL) was added. The mixture was concentrated under reduced pressure and methanol (5 mL) was added, and the mixture was stirred at 60° C. for 1 h. Ethylenediamine (1 mL) was then added and the mixture was stirred for 30 min. A saturated aqueous NaHCO₃ solution (20 mL) was added, and the mixture was extracted with CH₂Cl₂ (3×20 mL). The combined extract was dried over anhydrous Na₂SO₄. After filtration the solvent was removed to afford a sticky oil (0.207 g, 70% in 3 steps).

Following general procedure A, 4-(3-chloro-phenyl)-1-phenyl-3-piperidin-4-yl-imidazolidin-2-one (0.200 g, 0.811 mmol) and 1-boc-4-piperidone (0.179 g, 0.900 mmol) in CH₂Cl₂ (5 mL) and NaBH(OAc)₃ (0.074 mg, 0.35 mmol) gave the intermediate.

The product was dissolved in CH₂Cl₂ (5 mL) and cooled to 0° C. To the cooled solution was added pyridine (0.096 g, 1.22 mmol) and triphosgene (0.096 g, 0.324 mmol). The mixture was warmed to room temperature and stirred for 1 h. Standard work-up afforded a white foam.

Following general procedure C, the above carbamate afforded 4-(3-chloro-phenyl)-3-piperidin-4-yl-1-phenyl-3-yl-imidazolidin-2-one as a pale yellow foam (0.277 g, 96% in three steps).

COMPOUND 166 was isolated as an off-white solid (0.060 g, 64%). ¹H NMR (CD₃OD) δ 1.51-1.56 (m, 1H), 1.63-1.69 (m, 1H), 1.80-1.84 (m, 1H), 2.09-2.22 (m, 1H), 2.29-2.42 (m, 2H), 2.99-3.04 (m, 1H), 3.11-3.16 (m, 1H), 3.5 (dd, 1H, J=6.0, 9.0 Hz), 3.67-3.76 (m, 3H), 4.26 (t, 1H, J=9.0 Hz), 4.87-4.92 (m, 1H), 6.99 (d, 1H, J=8.4 Hz), 7.02-7.07 (m, 1H), 7.11-7.16 (m, 2H), 7.28-7.37 (m, 5H), 7.45-7.53 (m, 3H), 7.82 (dd, 1H, J=8.4, 2.4, Hz), 8.00-8.06 (m, 2H), 8.09 (d, 1H, J=2.4 Hz); ¹³C NMR (CD₃OD) δ 29.29, 30.74, 53.06, 53.55, 53.76, 54.95, 56.34, 59.25, 113.28, 119.66, 121.35, 124.34, 126.55, 127.86, 128.33, 129.89, 130.02, 130.53, 132.00, 132.75, 136.08, 141.35, 143.64, 145.99, 150.13, 159.23, 159.46, 164.41, 170.89; ES-MS m/z 558 (M+H). Anal. Calcd. for C₃₃H₃₁ClN₄O₄.0.2CH₂Cl₂: C, 66.45; H, 5.27; N, 9.34. Found: C, 66.65; H, 5.30; N, 9.36.

### EXAMPLE 167

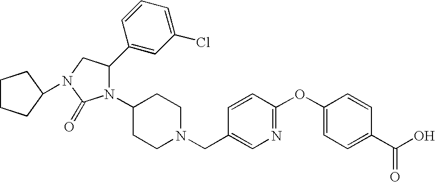

### COMPOUND 167: 4-(5-{4-[5-(3-Chloro-phenyl)-3-cyclopentyl-2-oxo-imidazolidin-1-yl]-piperidin-1-ylmethyl}-pyridin-2-yloxy)-benzoic acid

Following General Procedure E: using tert-butoxycarbonylamino-(3-chloro-phenyl)-acetic acid (0.340 g, 1.20 mmol), cyclopentylamine (0.129 g, 1.30 mmol), EDCI (0.289 g, 1.50 mmol), HOBT (0.203 g, 1.50 mmol) and DIPEA (0.258 g, 2.00 mmol). The crude product was purified by flash chromatography on silica gel (CH₂Cl₂) to afford the amide as a white solid (0.220 g).

Following General Procedure C: BOC-deprotection on the amide was carried out to afford a sticky oil (0.160 g).

The oil was dissolved in dry THF (6 mL), and BH₃-THF (1.0 M) (3.0 mL, 3.0 mmol) was added. After stirred at 65° C. overnight, the mixture was cooled to room temperature and methanol (1 mL) was added. After concentration, methanol (5 mL) was added, and the mixture was stirred at 60° C. for 1 h. Ethylenediamine (1 mL) was then added and the mixture was stirred for 30 min. A saturated aqueous NaHCO₃ solution (20 mL) was added, and the mixture was extracted with CH₂Cl₂ (3×20 mL). The combined extract was dried over anhydrous Na₂SO₄. After filtration the solvent was removed to afford 1-(3-chloro-phenyl)-N²-cyclopentyl-ethane-1,2-diamine as a sticky oil (0.098 g, 34% in 3 steps). ¹H NMR (CDCl₃) δ 1.26-1.32 (m, 2H), 1.50-1.72 (m, 7H), 1.77-1.85 (m, 2H), 2.65 (dd, 1H, J=12.0, 8.7 Hz), 2.80 (dd, 1H, J=12.0, 4.8 Hz), 3.06 (quintet, 1H, J=6.6 Hz), 3.98 (dd, 1H, J=9.7, 4.8 Hz), 7.20-7.26 (m, 2H), 7.35 (s, 1H).

Following General Procedure A: to a solution of 1-(3-chloro-phenyl)-N²-cyclopentyl-ethane-1,2-diamine (0.098 g, 0.41 mmol) and 1-boc-4-piperidone (0.100 g, 0.500 mmol) in CH₂Cl₂ (3 mL) was added NaBH(OAc)₃ (0.13 g, 0.60 mmol) and 1 drop AcOH, and the mixture stirred at room temperature overnight. The crude product was dissolved in CH₂Cl₂ (5 mL) and cooled at 0° C.

To the cooled solution was added pyridine (0.047 g, 0.60 mmol) and triphosgene (0.061 g, 0.20 mmol). The mixture was warmed to room temperature and stirred for 1 h. A saturated aqueous NaHCO₃ solution (15 mL) was added, and the mixture was extracted with CH₂Cl₂ (3×15 mL). The combined extract was dried over anhydrous Na₂SO₄. After filtration the solvent was removed, and the residue was purified by flash chromatography on silica gel (EtOAc/hexanes, 2:3 v/v) to afford the urea as a colorless oil (0.145 g).

Following General Procedure C: BOC-deprotection was carried out on the product to afford 4-(3-chloro-phenyl)-1-cyclopentyl-3-piperidin-4-yl-imidazolidin-2-one as a pale yellow foam (0.128 g, 88% in three steps). ¹H NMR (CDCl₃) δ 1.41-1.61 (m, 6H), 1.80-1.86 (m, 3H), 2.01-2.06 (m, 1H), 2.64-2.78 (m, 2H), 3.01-3.11 (m, 2H), 3.22-3.27 (m, 1H), 3.67 (t, 1H, J=9.0 Hz), 3.74-3.82 (m, 1H), 4.31 (quintet, 1H, J=7.8 Hz), 4.53-4.59 (m, 4H), 7.20-7.34 (m, 4H).

COMPOUND 167 was isolated as a white foam (0.047 g, 45% over 2 steps). ¹H NMR (CD₃OD) δ 1.57-1.82 (m, 11H), 2.24-2.36 (m, 1H), 2.77-2.88 (m, 2H), 3.10 (dd, 1H, J=9.0, 6.9 Hz), 3.35-3.41 (m, 2H), 3.36-3.64 (m, 1H), 3.79 (t, 1H, J=9.0 Hz), 4.11 (s, 2H), 4.23-4.30 (m, 1H), 4.73 (dd, 1H, J=9.0, 6.9 Hz), 7.08 (d, 1H, J=8.4 Hz), 7.17-7.20 (m, 2H), 7.32-7.41 (m, 4H), 7.91-7.94 (m, 1H), 8.05-8.08 (m, 2H), 8.18 (br s, 1H); ¹³C NMR (CD₃OD) δ 25.51, 28.47, 29.26, 29.82, 29.97, 51.79, 53.37, 53.49, 55.61, 57.84, 58.39, 113.89, 122.21, 123.76, 126.93, 128.62, 130.16, 132.35, 133.23, 136.37, 144.74, 146.22, 151.66, 159.46, 165.58; ES-MS m/z 575 (M+H).

### EXAMPLE 168

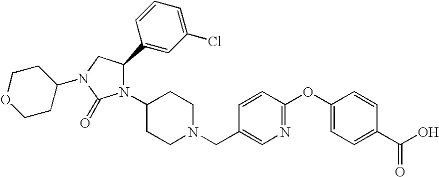

### COMPOUND 168: 4-(5-{4-[(R)-5-(3-Chloro-phenyl)-2-oxo-3-(tetrahydro-pyran-4-yl)-imidazolidin-1-yl]-piperidin-1-ylmethyl}-pyridin-2-yloxy)-benzoic acid

COMPOUND 168 was isolated as a white foam (0.165 g, 81% over 2 steps). ¹H NMR (CD₃OD) δ 1.47-1.75 (m, 7H), 2.04-2.15 (m, 1H), 2.30-2.35 (m, 2H), 2.98-3.14 (m, 3H), 3.42-3.50 (m, 2H), 3.60-3.80 (m, 4H), 3.94-3.98 (m, 3H), 4.73 (dd, 1H, J=9.0, 6.6 Hz), 6.95 (d, 1H, J=8.4 Hz), 7.09-7.12 (m, 2H), 7.26-7.32 (m, 3H), 7.37 (br s, 1H), 7.78-7.83 (m, 1H), 7.96-8.03 (m, 2H), 8.08 (br s, 1H); ¹³C NMR (CD₃OD) δ 29.12, 30.78, 31.02, 31.18, 50.62, 52.78, 53.44, 53.72, 54.97, 57.15, 59.13, 68.31, 113.15, 121.30, 126.51, 127.55, 128.15, 129.69, 131.85, 132.67, 135.94, 143.65, 146.25, 150.19, 158.88, 161.85, 164.50; ES-MS m/z 591 (M+H). Anal. Calcd. for C₃₂H₃₅ClN₄O₅.0.3CH₂Cl₂.0.2H₂O: C, 62.37; H, 5.87; N, 9.01. Found: C, 62.41; H, 5.94; N, 8.78.

### EXAMPLE 169

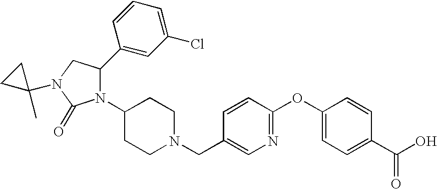

### COMPOUND 169: 4-(5-{4-[5-(3-Chloro-phenyl)-3-(1-methyl-cyclopropyl)-2-oxo-imidazolidin-1-yl]-piperidin-1-ylmethyl}-pyridin-2-yloxy)-benzoic acid

A solution of 1-methylcyclopropanecarboxylic acid (1.00 g, 10 mmol), diphenyl phosphoryl azide (4.31 mL, 20 mmol), Et₃N (2.8 mL, 20 mmol) and t-BuOH (5 mL) was heated at reflux overnight. Aqueous work-up afforded crude (1-methyl-cyclopropyl)-carbamic acid tert-butyl ester (2.93 g). The above substrate in MeOH.HCl (2 mL) was stirred at room temperature for 1 hour. The mixture was concentrated under reduced pressure and dried in vacuo for 1 hour to afford crude 1-methyl-cyclopropylamine hydrochloride (2.10 g).

Following general procedure F: to a solution of tert-butoxycarbonylamino-(3-chloro-phenyl)-acetic acid (429 mg, 1.50 mmol) in THF (16 mL) at 0° C. was added NMM (0.16 mL, 1.5 mmol) in THF (0.5 mL) followed by IBCF (0.19 mL, 1.5 mmol) and the mixture stirred for 15 minutes. The above amine (178 mg, 1.65 mmol) was added and the mixture stirred at 0° C. for 30 min and at room temperature overnight. Standard work-up and purification gave [(3-chloro-phenyl)-(1-methyl-cyclopropylcarbamoyl)-methyl]-carbamic acid tert-butyl ester (224 mg, 44%).

Following general procedure C with the above carbamate (224 mg, 0.662 mmol) gave the crude intermediate. Reduction with BH₃.THF (1.0M in THF, 2.5 mL, 2.5 mmol) in THF (6.7 mL) at reflux followed by treatment with 6N HCl (1 mL) and subsequent basic work-up and purification afforded 1-(3-chloro-phenyl)-N²-(1-methyl-cyclopropyl)-ethane-1,2-diamine (127 mg, 85% over 2 steps).

Following general procedure A, the above amine (127 mg, 0.566 mmol) and 1-BOC-4-piperidone (124 mg, 0.623 mmol) afforded the desired piperidine (88 mg, 38%). Following general procedure K: to a solution of the piperidine (88 mg, 0.22 mmol) and DIPEA (0.06 mL, 0.32 mmol) in CH₂Cl₂ (2 mL) at 0° C. was added a solution of triphosgene (30 mg, 0.11 mmol) in CH₂Cl₂ (1 mL) and the mixture was stirred at 0° C. for 1 hour and at 80° C. overnight. Standard work-up and purification afforded 4-[5-(3-chloro-phenyl)-3-(1-methyl-cyclopropyl)-2-oxo-imidazolidin-1-yl]-piperidine-1-carboxylic acid tert-butyl ester (16 mg, 17%). Following general procedure C, the above substrate (16 mg, 0.036 mmol) gave 4-(3-chloro-phenyl)-1-(1-methyl-cyclopropyl)-3-piperidin-4-yl-imidazolidin-2-one (13 mg, quant).

COMPOUND 169 was isolated as an orange solid (6.6 mg, 31% over 2 steps). ¹H NMR (CDCl₃) δ 0.52-0.74 (m, 2H), 0.78-0.92 (m, 2H), 1.01-1.98 (m, 6H), 2.12-2.81 (m, 2H), 3.06-3.30 (m, 2H), 3.41-4.12 (m, 3H), 4.50-4.69 (m, 1H), 6.96 (d, 1H, J=6.0 Hz), 7.13-7.35 (m, 6H), 8.03-8.09 (m, 4H); ¹³C NMR (CDCl₃) δ 12.3, 12.9, 18.1, 25.1, 27.3, 30.4, 48.5, 50.2, 50.6, 53.9, 56.0, 111.3, 119.9, 124.1, 125.4, 126.4, 127.7, 129.4, 130.8, 133.9, 141.6, 143.3, 148.5, 156.5, 159.1, 162.7, 167.7; ES-MS m/z 561 (M+1).

### EXAMPLE 170

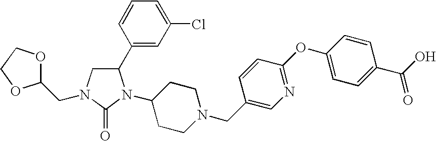

### COMPOUND 170: 4-(5-{4-[5-(3-Chloro-phenyl)-3-[3]dioxolan-2-ylmethyl-2-oxo-imidazolidin-1-yl]-piperidin-1-ylmethyl}-pyridin-2-yloxy)-benzoic acid

Following general procedure G: a solution of 2-bromomethyl-1,3-dioxolane (538 mg, 3.22 mmol), [2-amino-1-(3-chloro-phenyl)-ethyl]-carbamic acid tert-butyl ester (485 mg, 1.79 mmol), DIPEA (0.44 mL, 2.5 mmol) and KI (catalytic) in DMSO (2 mL) was heated to 90° C. for 25 hours. Standard work-up and purification afforded the desired product as a yellow oil (247 mg, 39%).

Following general procedure C with the above carbamate (247 mg, 0.692 mmol) and subsequently general procedure A with the resulting amine (166 mg, 0.647 mmol) and 1-BOC-4-piperidone (135 mg, 0.678 mmol) afforded the crude substrate (212 mg). Following general procedure K: to a solution of the above diamine (212 mg) and Et₃N (0.13 mL, 0.93 mmol) in CH₂Cl₂ (9 mL) at 0° C. was added a solution of triphosgene (57 mg, 0.19 mmol) in CH₂Cl₂ (1 mL). The mixture was stirred at room temperature for 1.5 hours. Standard work-up and purification afforded 4-[5-(3-chloro-phenyl)-3-[1,3]dioxolan-2-ylmethyl-2-oxo-imidazolidin-1-yl]-piperidine-1-carboxylic acid tert-butyl ester (218 mg). Following general procedure C afforded 4-(3-chloro-phenyl)-1-[1,3]dioxolan-2-ylmethyl-3-piperidin-4-yl-imidazolidin-2-one as a yellow foam (120 mg, 51% over 3 steps).

COMPOUND 170 was isolated as a yellow foam (56 mg, 63% over 2 steps). ¹H NMR (CD₃OD) δ 1.52-1.82 (m, 3H), 2.12-2.27 (m, 1H), 2.54-2.66 (m, 2H), 3.14-3.18 (m, 1H), 3.24-3.36 (m, 3H), 3.45 (dd, 1H, J=14.4, 3.9 Hz), 3.52-3.63 (m, 1H), 3.82-4.02 (m, 7H), 4.74 (dd, 1H, J=9.3, 6.3 Hz), 4.93-4.96 (m, 1H), 7.07 (d, 1H, J=8.4 Hz), 7.18 (d, 2H, J=8.7 Hz), 7.32-7.44 (m, 4H), 7.87 (dd, 1H, J=8.4, 2.4 Hz), 8.06 (d, 2H, J=9.0 Hz), 8.14 (d, 1H, J=2.1 Hz); ¹³C NMR (CD₃OD) δ 28.83, 29.86, 47.51, 52.27, 53.46, 53.64, 55.18, 57.68, 58.75, 66.39, 66.55, 104.09, 113.76, 121.97, 125.24, 126.92, 128.54, 129.77, 130.04, 132.15, 133.05, 136.28, 144.34, 146.32, 151.12, 159.52, 162.55, 165.20, 170.23; ES-MS m/z 593 (M+1). Anal. Calcd. for C₃₁H₃₃N₄ClO₆.0.5CH₂Cl₂.0.8CH₄O: C, 58.68; H, 5.67; N, 8.47. Found: C, 58.80; H, 5.51; N, 8.16.

### EXAMPLE 171

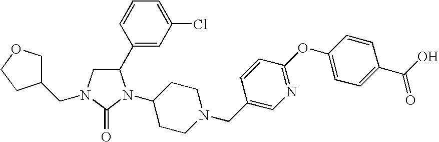

### COMPOUND 171: 4-(5-{4-[5-(3-Chloro-phenyl)-2-oxo-3-(tetrahydro-furan-4-ylmethyl)-imidazolidin-1-yl]-piperidin-1-ylmethyl}-pyridin-2-yloxy)-benzoic acid

See EXAMPLE 175 for preparation of 4-(3-chloro-phenyl)-3-piperidin-4-yl-1-(tetrahydro-furan-4-ylmethyl)-imidazolidin-2-one. COMPOUND 171 was isolated as a yellow foam (80 mg, 67% over 2 steps). ¹H NMR (CD₃OD) δ 1.58-2.08 (m, 10H), 2.25-2.38 (m, 2H), 2.46-2.57 (m, 2H), 2.81-2.93 (m, 4H), 3.14-3.23 (m, 6H), 3.30-3.92 (m, 16H), 4.15 (s, 4H), 4.74-4.79 (m, 2H), 7.11 (d, 2H, J=8.7 Hz), 7.19-7.22 (m, 4H), 7.35-7.43 (m, 8H), 7.93 (dd, 2H, J=8.4, 2.4 Hz), 8.05-8.09 (m, 4H), 8.19 (d, 2H, J=2.1 Hz); ¹³C NMR (CD₃OD) δ 28.24, 28.96, 31.16, 39.49, 48.06, 51.54, 53.20, 53.28, 54.22, 57.61, 57.68, 58.14, 69.05, 72.57, 113.88, 122.22, 123.14, 126.85, 128.51, 129.44, 130.11, 132.30, 133.08, 136.32, 144.80, 146.00, 146.06, 151.76, 159.43, 162.71, 165.55, 169.85; ES-MS m/z 591 (M+1). Anal. Calcd. for C₃₂H₃₅N₄ClO₅.0.8CH₂Cl₂: C, 59.78; H, 5.60; N, 8.50. Found: C, 59.60; H, 5.82; N, 8.40.

### EXAMPLE 172

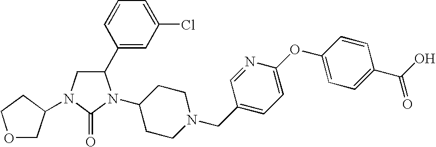

### COMPOUND 172: 4-(5-{4-[5-(3-Chloro-phenyl)-2-oxo-3-(tetrahydro-furan-3-yl)-imidazolidin-1-yl]-piperidin-1-ylmethyl}-pyridin-2-yloxy)-benzoic acid

COMPOUND 172 was prepared using the same chemistry as COMPOUND 167 except that 3-aminotetrahydrofuran hydrochloride was used in lieu of cyclopentylamine. COMPOUND 172 was isolated as a yellow solid. ¹H NMR (CDCl₃) δ 1.45-1.55 (m, 1H), 1.82-1.91 (m, 3H), 2.17-2.25 (m, 1H), 2.58-2.68 (m, 2H), 2.80-2.88 (m, 1H), 3.11-3.29 (m, 2H), 3.50-3.54 (m, 1H), 3.68-3.79 (m, 4H), 3.91-4.16 (m, 4H), 4.63-4.71 (m, 2H), 7.01 (d, 1H, J=8.4 Hz), 7.13 (d, 2H, J=8.4 Hz), 7.24-7.26 (m, 3H), 7.30-7.34 (m, 1H), 8.04 (d, 2H, J=8.4 Hz), 8.12 (br s, 1H), 8.30 (br d, 1H, J=8.1 Hz); ¹³C NMR (CDCl₃) δ 26.12, 27.90, 29.83, 30.14, 49.50, 51.89, 53.26, 67.88, 70.80, 71.15, 113.02, 120.56, 121.51, 125.49, 125.62, 126.64, 127.34, 129.26, 131.04, 132.26, 135.44, 143.54, 150.02, 157.82, 160.24, 164.36, 169.98; ES-MS m/z 578 (M+1). Anal. Calcd. for C₃₁H₃₃ClN₄O₅.1.02CH₂Cl₂: C, 57.93; H, 5.32; N, 8.44. Found: C, 57.93; H, 5.51; N, 8.39.

### EXAMPLE 173

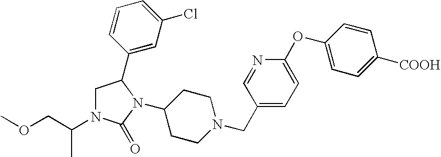

### COMPOUND 173: 4-(5-{4-[5-(3-Chloro-phenyl)-3-(2-methoxy-1-methyl-ethyl)-2-oxo-imidazolidin-1-yl]-piperidin-1-ylmethyl}-pyridin-2-yloxy)-benzoic acid

Following general procedure A: to a solution of [2-amino-1-(3-chloro-phenyl)-ethyl]-carbamic acid tert-butyl ester (350 mg, 1.3 mmol) in DCM (9 mL) was added 1-methoxy-propan-2-one (0.15 mL, 1.7 mmol). After 30 minutes of stirring at room temperature, NaBH(OAc)₃ (381 mg, 1.8 mmol) was added and the reaction mixture was allowed to stir overnight (˜19 hours).

Standard work-up and purification afforded [1-(3-chloro-phenyl)-2-(2-methoxy-1-methyl-ethylamino)-ethyl]-carbamic acid tert-butyl ester (295 mg, 66%).

Following general procedure C, the above carbamate (295 mg, 0.86 mmol) afforded 1-(3-chloro-phenyl)-N²-(2-methoxy-1-methyl-ethyl)ethane-1,2-diamine as a yellow oil (153 mg, 73%).

Following general procedure A: to a solution of the above amine (153 mg, 0.63 mmol) in DCM (3 mL) was added N-boc piperidone (163 mg, 0.819 mmol) followed by NaBH(OAc)₃ (266 mg, 1.3 mmol) and the mixture was stirred at rt for 19 hours. Standard work-up and purification afforded 4-[1-(3-chloro-phenyl)-2-(2-methoxy-1-methyl-ethylamino)-ethylamino]-piperidine-1-carboxylic acid tert-butyl ester (167 mg, 62%).

Following general procedure K: to a solution of the above diamine (167 mg, 0.39 mmol) in DCM (4 mL) at 0° C. was added pyridine (63 μL, 0.78 mmol) followed by triphosgene (59.4 mg, 0.20 mmol) and the mixture was stirred at 0° C. for 40 minutes. Standard work-up and purification afforded 4-[5-(3-chloro-phenyl)-3-(2-methoxy-1-methyl-ethyl)-2-oxo-imidazolidin-1yl]-piperidine-1-carboxylic acid tert-butyl ester (110 mg, 62%).

Following general procedure C, the above substrate (110 mg, 0.24 mmol) afforded 4-(3-chloro-phenyl)-1-(2-methoxy-1-methyl-ethyl)-3-piperidin-4-yl-imidazolidin-2-one (80 mg, 94%).

COMPOUND 173 was isolated as a yellow/brown solid (35.5 mg, 30% over 2 steps). ¹H NMR (CDCl₃) δ 1.10 (d, 3H, J=6.0 Hz), 1.20-1.39 (m, 1H), 1.44-1.53 (m, 1H), 1.75-1.79 (m, 1H), 2.08-2.17 (m, 2H), 2.24-2.28 (m, 1H), 2.99-3.08 (m, 2H), 3.27-3.50 (m, 7H), 3.59 (d, 1H, J=12.9 Hz), 3.68 (t, 1H, J=9.1 Hz), 4.23-4.38 (m, 1H), 4.50-4.60 (m, 1H), 6.82 (d, 1H, J=8.4 Hz), 7.05-7.15 (m, 5H), 7.29 (s, 1H), 7.60 (dd, 1H, J=8.4, 2.1 Hz), 7.99 (d, 2H, J=8.4 Hz), 8.08 (s, 1H); ¹³C NMR (CDCl₃) δ 14.6, 27.8, 30.3, 47.3, 47.5, 48.6, 49.0, 51.2, 51.4, 50.6, 52.7, 55.5, 55.7, 58.8, 49.2, 74.4, 74.7, 111.7, 120.8, 125.3, 125.5, 125.9, 127.0, 127.2, 128.8, 129.3, 130.4, 130.5, 132.0, 135.1, 142.1, 145.3, 145.7, 149.7, 157.9, 160.4, 160.6, 163.5, 169.7; ES-MS m/z 579 (M+H). Anal. Calcd. for C₃₁H₃₅N₄ClO₅.0.7CH₂Cl₂: C, 59.93; H, 5.77; N, 8.83. Found: C, 59.97; H, 5.71; N, 8.66.

Examples 174 to 179 were prepared following the scheme illustrated below. RCH₂Br is as defined in the table and X is as defined in the individual examples.

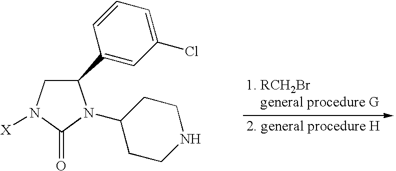

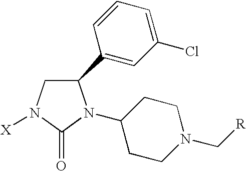

| Example | RCH₂Br |
|---|---|
| 174 | 4-(4-bromomethyl-phenoxy)-benzoic acid methyl ester |
| 175 | 4-(4-bromomethyl-phenoxy)-benzoic acid methyl ester |
| 176 | 4-(5-bromomethyl-pyridin-2-yloxy)-cyclohexanecarboxylic |
| | acid ethyl ester |
| 177 | 4-(5-bromomethyl-pyridine-2-carbonyl)-benzoic acid methyl |
| | ester |
| 178 | 4-(5-bromomethyl-pyridin-2-ylsulfanyl)-benzoic acid methyl |
| | ester |
| 179 | 4-(5-bromomethyl-pyridin-2-ylsulfanyl)-benzoic acid methyl |
| | ester |

### EXAMPLE 174

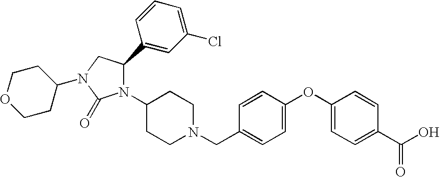

### COMPOUND 174: 4-(4-{4-[(R)-5-(3-Chloro-phenyl)-2-oxo-3-(tetrahydro-pyran-4-yl)-imidazolidin-1-yl]-piperidin-1-ylmethyl}-phenoxy)-benzoic acid

COMPOUND 174 was isolated as a white foam (0.039 g, 56% over 2 steps). ¹H NMR (CD₃OD) δ 1.45-1.78 (m, 7H), 2.12-2.18 (m, 1H), 2.39-2.52 (m, 2H), 3.07-3.21 (m, 3H), 3.43-3.51 (m, 2H), 3.60-3.71 (m, 1H), 3.76-3.82 (m, 3H), 3.93-3.99 (m, 3H), 4.74 (dd, 1H, J=9.0, 6.6 Hz), 6.95-7.03 (m, 4H), 7.29-7.40 (m, 6H), 7.94-7.98 (m, 2H); ¹³C NMR (CD₃OD) δ 28.45, 29.80, 31.01, 31.20, 50.63, 52.10, 53.09, 53.37, 57.28, 61.44, 68.29, 68.35, 118.96, 120.60, 126.53, 128.18, 129.00, 129.75, 131.66, 131.91, 132.80, 133.78, 135.98, 146.04, 158.69, 161.21, 161.75; ES-MS m/z 590 (M+H).

### EXAMPLE 175

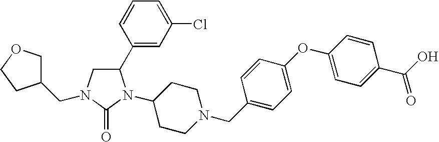

### COMPOUND 175: 4-(4-{4-[5-(3-Chloro-phenyl)-2-oxo-3-(tetrahydro-furan-3-ylmethyl)-imidazolidin-1-yl]-piperidin-1-ylmethyl}-phenoxy)-benzoic acid

Following general procedure F: to a solution of tetrahydro-furan-4-carboxylic acid (0.08 mL, 0.84 mmol) in THF (4.2 mL) at 0° C. was added NMM (85 mg, 0.84 mmol) in THF (0.5 mL) followed by IBCF (0.11 mL, 0.84 mmol) and the mixture stirred for 15 minutes. [2-Amino-1-(3-chloro-phenyl)-ethyl]-carbamic acid tert-butyl ester (450 mg, 1.66 mmol) was added and the mixture stirred at 0° C. for 30 min and at room temperature for 3 hours. Standard work-up and purification gave {1-(3-chloro-phenyl)-2-[(tetrahydro-furan-4-carbonyl)-amino]-ethyl}-carbamic acid tert-butyl ester (295 mg, 95%).

Following general procedure C with the above carbamate (295 mg, 0.80 mmol) gave the crude intermediate. Reduction with BH₃.THF (1.0M in THF, 3.2 mL, 3.2 mmol) in THF (8 mL) at reflux followed by treatment with MeOH (8 mL) and then 6N HCl (4.5 mL) and subsequent basic work-up and purification afforded 1-(3-chloro-phenyl)-N²-(tetrahydro-furan-4-ylmethyl)-ethane-1,2-diamine as a colourless oil (179 mg, 88% over 2 steps).

Following general procedure A, the above amine (179 mg, 0.70 mmol) and 1-BOC-4-piperidone (154 mg, 0.77 mmol) afforded the desired piperidine as a yellow oil (204 mg, 66%). Following general procedure K: to a solution of the piperidine (204 mg, 0.61 mmol) and Et₃N (0.17 mL, 1.2 mmol) in CH₂Cl₂ (12 mL) at 0° C. was added a solution of triphosgene (67 mg, 0.22 mmol) in CH₂Cl₂ (1 mL) and the mixture was stirred a room temperature for 3 hours. Standard work-up and purification afforded 4-[5-(3-chloro-phenyl)-2-oxo-3-(tetrahydro-furan-4-ylmethyl)-imidazolidin-1-yl]-piperidine-1-carboxylic acid tert-butyl ester (209 mg, 97%).

Following general procedure C, the above substrate (209 mg, 0.57 mmol) gave crude 4-(3-chloro-phenyl)-3-piperidin-4-yl-1-(tetrahydro-furan-4-ylmethyl)-imidazolidin-2-one (144 mg).

COMPOUND 175 was isolated as a yellow foam (70 mg, 56% over 2 steps). ¹H NMR (CD₃OD) δ 1.58-2.07 (m, 10H), 2.27-2.40 (m, 2H), 2.46-2.59 (m, 2H), 2.85-2.97 (m, 4H), 3.14-3.23 (m, 6H), 3.30-3.92 (m, 16H), 4.16 (s, 4H), 4.75-4.81 (m, 2H), 7.01-7.06 (m, 4H), 7.11 (d, 4H, J=8.7 Hz), 7.33-7.49 (m, 12H), 8.00-8.03 (m, 4H); ¹³C NMR (CD₃OD) δ 29.18, 28.91, 31.15, 39.54, 48.05, 51.61, 53.13, 53.27, 54.20, 57.78, 60.97, 69.04, 72.57, 119.48, 121.39, 126.85, 126.97, 128.55, 130.13, 132.29, 133.41, 134.72, 136.34, 145.93, 159.33, 162.35, 162.73, 170.22; ES-MS m/z 590 (M+1). Anal. Calcd. for C₃₃H₃₆N₃ClO₅.0.5CH₂Cl₂.0.7H₂O: C, 62.36; H, 6.00; N, 6.51. Found: C, 62.30; H, 6.04; N, 6.46.

### EXAMPLE 176

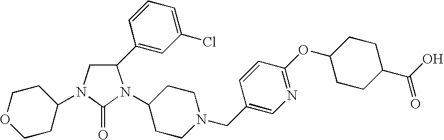

### COMPOUND 176: 4-(5-{4-[5-(3-Chloro-phenyl)-2-oxo-3-(tetrahydro-pyran-4-yl)-imidazolidin-1-yl]-piperidin-1-ylmethyl}-pyridin-2-yloxy)-cyclohexanecarboxylic acid

To a mixture of 2-hydroxy-5-methylpyridine (411 mg, 3.77 mmol), ethyl 4-hydroxycyclohexanecarboxylate (0.607 mL, 3.77 mmol) and triphenylphosphine (988 mg, 3.77 mmol) in THF (25 mL) cooled to 0° C. was added DIAD (0.73 mL, 3.8 mmol). The mixture was stirred at room temperature for 3 days. Standard work-up and purification afforded 4-(5-methyl-pyridin-2-yloxy)-cyclohexanecarboxylic acid ethyl ester (339 mg, 34%).

N₂ was bubbled through a solution of the above ether (115 mg, 0.437 mmol) in CCl₄ (3 mL), then benzoyl peroxide (11 mg, 0.045 mmol) and N-bromosuccinimide (77 mg, 0.45 mmol) were added. The mixture was heated to 90° C. for 2 hours. Hexanes was added, the mixture was filtered and the filtrant was concentrated under reduced pressure to afford crude 4-(5-bromomethyl-pyridin-2-yloxy)-cyclohexanecarboxylic acid ethyl ester (188 mg).

COMPOUND 176 was isolated as a white solid (61 mg, 23% over 2 steps). ¹H NMR (CDCl₃) δ 1.39-2.50 (m, 19H), 3.03-3.12 (m, 2H), 3.30-3.32 (m, 1H), 3.41-3.50 (m, 2H), 3.65-4.07 (m, 7H), 4.61 (dd, 1H, J=9.0, 6.0 Hz), 4.90-4.99 and 5.17-5.20 (m, 1H), 6.64 and 6.70 (d, 1H, J=8.7 Hz), 7.19-7.30 (m, 4H), 7.64 and 7.70 (d, 1H, J=8.1 Hz), 7.99-8.02 (m, 1H); ¹³C NMR (CDCl₃) δ 21.69, 24.38, 26.81, 27.45, 28.85, 29.43, 30.34, 31.22, 42.79, 48.64, 49.20, 50.46, 51.87, 53.83, 55.40, 57.93, 64.48, 67.52, 73.59, 111.99, 120.07, 125.32, 126.88, 128.62, 129.05, 130.80, 135.28, 141.43, 144.89, 149.32, 160.10, 164.08; ES-MS m/z 597 (M+1). Anal. Calcd. for C₃₂H₄₁ClN₄O₅.0.76CH₂Cl₂: C, 59.45; H, 6.48; N, 8.42. Found: C, 59.48; H, 6.69; N, 8.20.

### EXAMPLE 177

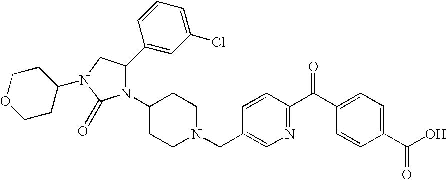

### COMPOUND 177: 4-(5-{4-[5-(3-Chloro-phenyl)-2-oxo-3-(tetrahydro-pyran-4-yl-imidazolidin-1-yl]-piperidin-1-ylmethyl}-pyridine-2-carbonyl)-benzoic acid

To a solution of 2-bromo-5-methylpyridine (1.06 g, 6.1 mmol) in diethyl ether (30 mL) at −78° C. under an atmosphere of argon was added s-butyl lithium (5.25 mL, 1.4 M in hexanes, 7.4 mmol). The solution was stirred for 30 minutes at this temperature, and then transferred by canula to a solution of 4-formyl-benzoic acid methyl ester (0.91 g, 5.5 mmol) in 1:1 diethyl ether/THF (20 mL) also at −78° C. The reaction mixture was stirred for 1 h, while gradually warming to ambient temperature. Aqueous work-up and isolation afforded crude 4-[hydroxyl-(5-methyl-pyridin-2-yl)-methyl]-benzoic acid methyl ester (1.45 g) which was used immediately in the next reaction.

The above alcohol (1.42 g, 5.5 mmol) was dissolved in CH₂Cl₂ (30 mL) and treated with manganese dioxide (2.40 g, 27.6 mmol) for 16 h at 40° C. The mixture was filtered through a Celite® pad washing with an adequate volume of CH₂Cl₂. The solvent was removed under reduced pressure and the crude residue purified by column chromatography on silica gel (1:5, EtOAc/hexanes) to afford 4-(5-methyl-pyridine-2-carbonyl)-benzoic acid methyl ester (0.86 g, 60% 2 steps). ¹H NMR (CDCl₃) δ 2.45 (s, 3H), 3.95 (s, 3H), 7.71 (d, 1H, J=7.5 Hz), 8.02 (d, 1H, J=7.5 Hz), 8.11 (m, 4H), 8.54 (s, 1H).

A solution of the above ketone (0.86 g, 3.4 mmol) and N-bromosuccinimide (0.66 g, 3.7 mmol) in CCl₄ (17 mL) was treated with benzoyl peroxide (82 mg, 0.34 mmol) and stirred at reflux for 16 h. The reaction was cooled to room temperature and partitioned in a separatory funnel between saturated aqueous sodium chloride solution (20 mL) and CH₂Cl₂ (25 mL). The aqueous phase was washed with CH₂Cl₂ (3×20 mL) and the combined organic dried (Na₂SO₄), filtered and concentrated under reduced pressure to afford, after column chromatography on silica gel (50:1 hexane/EtOAc), 4-(5-bromomethyl-pyridine-2-carbonyl)-benzoic acid methyl ester (0.89 g, 79%).

COMPOUND 177 was isolated as a white solid (44 mg, 47% over 2 steps). ¹H NMR (CD₃OD) δ 1.39 (q, 1H, J=10.8, 3.3 Hz), 1.50-1.80 (m, 6H), 1.95-2.25 (m, 3H), 2.85 (d, 1H, J=10.8 Hz), 2.98 (br d, 1H, J=10.8 Hz), 3.10 (m, 1H), 3.47 (t, 2H, J=11.4 Hz), 3.57 (m, 1H), 3.66 (s, 2H), 3.78 (t, 1H, J=9.0 Hz), 3.94 (m, 3H), 4.74 (m, 1H), 7.25-7.45 (m, 4H), 7.85-8.00 (m, 2H), 8.00 (d, 2H, J=8.4 Hz), 8.09 (d, 2H, J=8.4 Hz), 8.59 (s, 1H); ES-MS m/z 603 (M+H).

### EXAMPLE 178

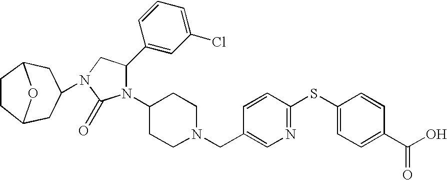

### COMPOUND 178: 4-(5-{4-[5-(3-Chloro-phenyl)-3-(8-oxa-bicyclo[3.2.1]oct-3-yl)-2-oxo-imidazolidin-1-yl]-piperidin-1-ylmethyl}-pyridin-2-ylsulfanyl)-benzoic acid

Following General Procedure A: using [2-amino-1-(3-chloro-phenyl)-ethyl]-carbamic acid tert-butyl ester (0.690 g, 2.55 mmol), 8-oxa-bicyclo[3.2.1]octan-3-one (0.330 g, 2.62 mmol) (Kim, H., Hoffmann, H. M. R., Eur. J. Org. Chem. 12, 2000, 2195-2202. Ansell, Martin F., Mason, Jonathan S., Caton, Michael P. L., J. Chem. Soc. Perkin Trans. 1, 5, 1984, 1061-1068.) and 5 drops AcOH. The crude product was purified by flash chromatography on silica gel (CH₃OH/CH₂Cl₂, 1:20 v/v) to afford a pale yellow oil (0.730 g, impure).

Following General Procedure C: BOC-deprotection was carried out on the above product to afford a sticky oil (0.425 g, impure).

Following General Procedure A: to a solution of the above oil (0.425 g, impure) and 1-boc-4-piperidone (0.400 g, 2.00 mmol) in CH₂Cl₂ (5 mL) was added NaBH(OAc)₃ (0.480 g, 2.25 mmol) and 2 drops AcOH, and the mixture stirred at room temperature overnight. The crude product was dissolved in CH₂Cl₂ (5 mL) and cooled to 0° C. To the cooled solution was added DIPEA (0.258 g, 2.00 mmol) and triphosgene (0.333 g, 1.10 mmol). The mixture was warmed to room temperature and stirred for 1 h. A saturated aqueous NaHCO₃ solution (15 mL) was added, and the mixture was extracted with CH₂Cl₂ (3×20 mL). The combined extract was dried over anhydrous Na₂SO₄. After filtration the solvent was removed, and the residue was purified by flash chromatography on silica gel (CH₃OH/CH₂Cl₂, 1:20 v/v).

Following General Procedure C: BOC-deprotection was carried out on the product in the last step to afford 4-(3-chloro-phenyl)-1-(8-oxa-bicyclo[3.2.1]oct-3-yl)-3-piperidin-4-yl-imidazolidin-2-one as a pale yellow foam (0.340 g, 34% over five steps). ¹H NMR (CDCl₃) δ 1.08-1.14 (m, 1H), 1.44-1.80 (m, 7H), 1.91-1.96 (m, 2H), 2.10-2.29 (m, 2H), 2.52 (dt, 1H, J=12.3, 2.4 Hz), 2.63 (dt, 1H, J=12.3, 3.0 Hz), 2.92-3.00 (m, 2H), 3.02-3.13 (m, 1H), 3.60 (t, 1H, J=8.7 Hz), 3.72-3.80 (m, 1H), 3.98-4.03 (m, 1H), 4.40-4.45 (m, 2H), 4.54 (dd, 1H, J=9.3, 6.9 Hz), 7.19-7.32 (m, 4H).

COMPOUND 178 was isolated as a white foam (0.099 g, 85% over 2 steps). ¹H NMR (CD₃OD) δ 1.47-1.55 (m, 2H), 1.59-1.92 (m, 7H), 2.13-2.27 (m, 3H), 2.71-2.82 (m, 2H), 3.09 (dd, 1H, J=9.0, 6.6 Hz), 3.22-3.34 (m, 2H), 3.4-3.62 (m, 1H), 3.73 (t, 1H, J=9.3 Hz), 3.97-4.03 (m, 1H), 4.04 (s, 2H), 4.38-4.02 (m, 2H), 4.71 (dd, 1H, J=9.3, 6.6 Hz), 7.13 (d, 1H, J=8.4 Hz), 7.30-7.40 (m, 4H), 7.62 (d, 1H, J=7.2 Hz), 7.69 (dd, 1H, J=8.4, 2.1 Hz), 8.05 (d, 2H, J=7.2 Hz), 8.40 (d, 1H, J=12.1 Hz); ES-MS m/z 633 (M+H). Anal. Calcd. for C₃₄H₃₇ClN₄O₄S.0.4CH₂Cl₂.2.0H₂O: C, 58.76; H, 5.99; N, 7.97; Cl, 9.07. Found: C, 59.05; H, 6.10; N, 7.83; Cl, 8.87.

### EXAMPLE 179

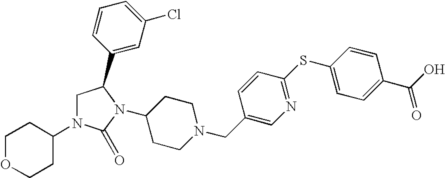

### COMPOUND 179: 4-(5-{4-[(R)-5-(3-Chloro-phenyl)-2-oxo-3-(tetrahydro-pyran-4-yl)-imidazolidin-1-yl]-piperidin-1-ylmethyl}-pyridin-2-ylsulfanyl)-benzoic acid

COMPOUND 179 was isolated as a white solid (412 mg, 86% over 2 steps). ¹H NMR (CDCl₃) δ 1.44 (br d, 1H, J=12.3 Hz), 1.63-1.67 (m, 3H), 1.90-2.04 (m, 2H), 2.60-2.80 (m, 4H), 3.12 (dd, 1H, J=9.3, 5.6 Hz), 3.28 (br d, 1H, J=11.4 Hz), 3.44-3.54 (m, 3H), 3.73 (t, 1H, J=9.6 Hz), 3.99-4.10 (m, 6H), 4.69 (dd, 1H, J=9.0, 6.0 Hz), 7.10 (d, 1H, J=8.4 Hz), 7.23-7.30 (m, 3H), 7.34 (s, 1H), 7.60 (d, 2H, J=8.4 Hz), 8.03 (d, 2H, J=8.4 Hz), 8.19 (d, 1H, J=7.5 Hz), 8.33 (s, 1H); ES-MS m/z 607 (M+H). Anal. Calcd. for C₃₂H₃₅N₄ClO₄S.0.8CH₂Cl₂.0.8CH₃OH: C, 57.59; H, 5.72; N, 8.00. Found: C, 57.64; H, 5.88; N, 8.34.

Examples 180 to 185 were prepared following the scheme illustrated below. RCHO is as defined in the table and X is as defined in the individual examples.

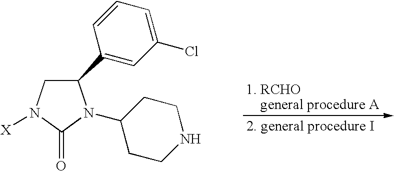

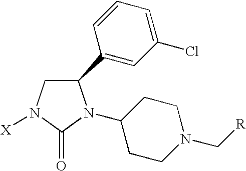

| Example | RCHO |
|---|---|
| 180 | 4-(5-formyl-pyridin-2-yloxy)-3-methyl-benzonitrile |
| 181 | 4-(5-formyl-pyridin-2-yloxy)-2-methyl-benzonitrile |
| 182 | 5-(5-formyl-pyridin-2-yloxy)-pyridine-2-carbonitrile |
| 183 | 4-(5-formyl-6-methyl-pyridin-2-yloxy)-3-methyl-benzonitrile |
| 184 | 4-(5-formyl-4-methyl-pyrimidin-2-ylamino)-benzonitrile |
| 185 | 4-(5-formyl-4-methyl-pyrimidin-2-ylamino)-benzonitrile |

### EXAMPLE 180

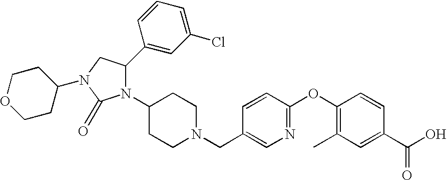

### COMPOUND 180: 4-(5-{4-[5-(3-Chloro-phenyl)-2-oxo-3-(tetrahydro-pyran-4-yl)-imidazolidin-1-yl]-piperidin-1-ylmethyl}-pyridin-2-yloxy)-3-methyl-benzoic acid

COMPOUND 180 was isolated as a white foam (0.120 g, 72% over 2 steps). ¹H NMR (CD₃Cl) δ 1.36-1.53 (m, 2H), 1.60-1.67 (m, 3H), 1.71-1.79 (m, 1H), 2.16-2.30 (m, 5H), 3.01-3.07 (m, 2H), 3.24-3.28 (m, 1H), 3.43-3.58 (m, 6H), 3.65 (t, 1H, J=9.3 Hz), 3.87-4.10 (m, 4H), 4.57 (dd, J=9.0, 6.6 Hz), 6.78 (d, 1H, J=8.4 Hz), 7.03 (d, 1H, J=8.1 Hz), 7.09-7.15 (m, 3H), 7.29 (s, 1H), 7.63 (br s, 1H), 7.79 (dd, 1H, J=8.4, 1.8 Hz), 7.89 (s, 1H), 8.03 (d, 1H, J=1.8 Hz); ¹³C NMR (CD₃OD) δ 16.59, 29.05, 30.56, 31.02, 31.21, 50.65, 52.67, 53.42, 53.70, 57.28, 59.06, 68.32, 68.37, 112.27, 122.28, 126.54, 128.20, 129.72, 130.18, 131.66, 131.88, 134.17, 135.97, 143.72, 146.16, 150.28, 157.00, 161.85, 164.72; ES-MS m/z 605 (M+H). Anal. Calcd. for C₃₃H₃₇ClN₄O₅.0.35CH₂Cl₂.0.9H₂O: C, 61.52; H, 6.11; N, 8.60; Cl, 9.26. Found: C, 61.52; H, 6.02; N, 8.45; Cl, 9.42.

### EXAMPLE 181

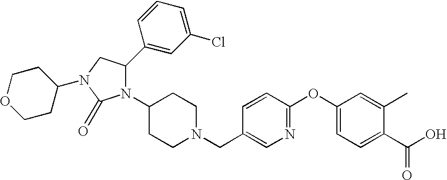

### COMPOUND 181: 4-(5-{4-[5-(3-Chloro-phenyl)-2-oxo-3-(tetrahydro-pyran-4-yl)-imidazolidin-1-yl]-piperidin-1-ylmethyl}-pyridin-2-yloxy)-2-methyl-benzoic acid

COMPOUND 181 was isolated as a white foam (0.053 g, 36% over 2 steps). ¹H NMR (CD₃OD) δ 1.63-1.87 (m, 7H), 2.22-2.34 (m, 1H), 2.56 (s, 3H), 2.71-2.82 (m, 2H), 3.11 (dd, 1H, J=8.7, 7.2 Hz), 3.23-3.38 (m, 2H), 3.46-3.50 (m, 2H), 3.58-3.66 (m, 1H), 3.79 (t, 1H, J=9.3 Hz), 3.88-3.99 (m, 3H), 4.06 (s, 2H), 4.75 (dd, 1H, J=9.3, 6.9 Hz), 6.95-7.05 (m, 3H), 7.31-7.37 (m, 2H), 7.41 (s, 1H), 7.90-7.94 (m, 2H), 8.17 (s, 1H); ¹³C NMR (CD₃OD) δ 22.19, 28.31, 29.11, 30.95, 31.23, 50.63, 51.67, 53.09, 53.23, 57.64, 58.23, 68.28, 68.35, 113.38, 119.13, 123.87, 124.69, 126.58, 128.29, 129.08, 129.85, 132.00, 133.79, 136.03, 143.72, 144.21, 145.73, 151.10, 157.87, 161.68, 165.22, 171.08; ES-MS m/z 605 (M+H). Anal. Calcd. for C₃₃H₃₇ClN₄O₅.0.55CH₂Cl₂.1.3H₂O: C, 59.68; H, 6.07; N, 8.30; Cl, 11.03. Found: C, 59.60; H, 6.00; N, 8.07; Cl, 11.06.

### EXAMPLE 182

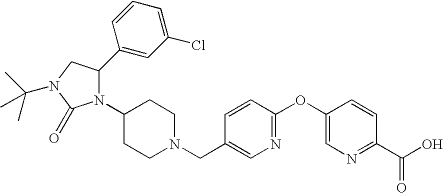

### COMPOUND 182: 5-(5-{4-[3-tert-Butyl-5-(3-chloro-phenyl)-2-oxo-imidazolidin-1-yl]-piperidin-1-ylmethyl}-pyridin-2-yloxy)-pyridine-2-carboxylic acid

Under N₂, to a dry flask charged with 6-(6-chloro-pyridin-3-yloxy)-pyridine-3-carbaldehyde (0.769 g, 3.27 mmol), Zn(CN)₂ (0.315 g, 2.68 mmol), dppf (0.075 g, 0.135 mmol), Pd₂(dba)₃ (0.050 g, 0.055 mmol) and Zn dust (0.042 g, 0.65 mmol) was added anhydrous N,N-dimethylacetamide (15 mL). The mixture was stirred at 130° C. for 3 h and then cooled to room temperature. The solvent was removed, brine (50 mL) was added and the mixture was extracted with EtOAc (2×50 mL). The combined extract was dried over anhydrous Na₂SO₄. After filtration the solvent was removed, and the residue was purified by flash chromatography on silica gel (EtOAc/hexanes, 1:1 in v/v) to afford 5-(5-formyl-pyridin-2-yloxy)-pyridine-2-carbonitrile as a yellow solid (0.505 g, 68%). ¹H NMR (CDCl₃) δ 7.21 (d, 1H, J=8.4 Hz), 7.74 (dd 1H, J=8.4, 2.4 Hz), 7.80 (d, 1H, J=8.4 Hz), 8.29 (dd, 1H, J=8.4, 2.4 Hz), 8.60 (d, 1H, J=2.4 Hz), 8.65 (d, 1H, J=2.4 Hz).

COMPOUND 182 was isolated as a white foam (0.023 g, 38% over 2 steps). ¹H NMR (CD₃OD) δ 1.27-1.30 (m, 1H), 1.35 (s, 9H), 1.45-1.53 (m, 1H), 1.67-1.71 (m, 1H), 1.92-2.02 (m, 1H), 2.13-2.28 (m, 2H), 2.86-2.90 (m, 1H), 3.03-3.14 (m, 2H), 3.55-3.62 (m, 3H), 3.75-3.81 (m, 1H), 4.61 (dd, 1H, J=9.0, 6.6 Hz), 6.98 (d, 1H, J=8.1 Hz), 7.29 (br s, 3H), 7.37 (s, 1H), 7.59 (br s, 1H), 7.76 (d, 1H, J=8.1 Hz), 7.97 (br s, 1H), 8.04 (br s, 1H), 8.39 (br s, 1H); ES-MS m/z 564 (M+H).

### EXAMPLE 183

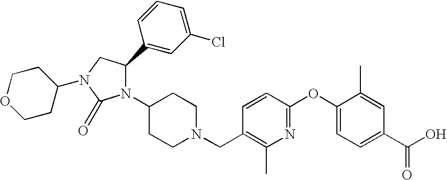

### COMPOUND 183: 4-(5-{4-[(R)-5-(3-Chloro-phenyl)-2-oxo-3-(tetrahydro-pyran-4-yl)-imidazolidin-1-yl]-piperidin-1-ylmethyl}-6-methyl-pyridin-2-yloxy)-3-methyl-benzoic acid

COMPOUND 183 was isolated as a pale orange powder (44.9 mg, 52% over 2 steps). ¹H NMR (CDCl₃) δ 1.47 (m, 1H), 1.59-1.70 (m, 4H), 1.76-1.80 (m, 1H), 2.13-2.50 (m, 2H), 2.24 (s, 3H), 2.41 (s, 3H), 2.92 (m, 1H), 2.98 (t, 1H, J=7.5 Hz), 3.23 (m, 1H), 3.35-3.52 (m, 3H), 3.68 (t, 2H, J=8.7 Hz), 3.71-4.10 (m, 6H), 4.60 (m, 1H), 6.51 (br s, 1H), 7.01 (d, 1H, J=7.5 Hz), 7.19 (br s, 3H), 7.31 (s, 1H), 7.82 (d, 1H, J=7.2 Hz), 7.94 (s, 1H); ¹³C NMR (CDCl₃) δ 16.8, 22.6, 27.5, 30.3, 48.6, 49.2, 50.9, 52.7, 55.5, 57.7, 67.5, 108.5, 121.0, 125.4, 126.8, 128.4, 129.0, 129.5, 130.6, 130.8, 133.7, 135.3, 143.4, 144.9, 156.6, 157.4, 160.2, 162.5; ES-MS m/z 619 (M+H). Anal. Calcd. for C₃₄H₃₉N₄O₅Cl.1.7CH₂Cl₂: C, 56.16; H, 5.60; N, 7.34. Found: C, 56.1 1; H, 5.47; N, 7.22.

### EXAMPLE 184

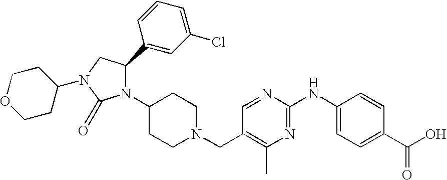

### COMPOUND 184: 4-(5-{4-[(R)-5-(3-Chloro-phenyl)-2-oxo-3-(tetrahydro-pyran-4-yl)-imidazolidin-1-yl]-piperidin-1-ylmethyl}-4-methyl-pyrimidin-2-ylamino)-benzoic acid

COMPOUND 184 was isolated as a brown solid (0.036 g, 22% over 2 steps). ¹H NMR (CD₃OD) δ 1.65-1.94 (m, 7H), 2.32-2.37 (m, 1H), 2.55 (s, 3H), 2.92-2.99 (m, 2H), 3.17 (dd, 1H, J=9.0, 7.2 Hz), 3.36-3.39 (m, 1H), 3.47-3.55 (m, 3H), 3.61-3.65 (m, 1H), 3.83 (t, 1H, J=9.0 Hz), 3.95-4.03 (m, 3H), 4.14 (s, 2H), 4.79 (dd, 1H, J=9.0, 6.9 Hz), 7.38-7.46 (m, 4H), 7.86-7.89 (m, 2H), 7.97-8.00 (m, 2H), 8.42 (s, 1H); ¹³C NMR (CD₃OD) δ 22.66, 28.09, 28.74, 30.94, 31.22, 50.62, 51.36, 53.07, 53.20, 55.84, 57.65, 68.26, 68.34, 115.11, 119.39, 126.59, 128.28, 129.86, 131.87, 132.01, 136.02, 145.64, 145.89, 161.26, 161.62, 162.26, 170.39; ES-MS m/z 605 (M+H). Anal. Calcd. for C₃₂H₃₇N₆O₄.0.9CH₂Cl₂.1.9H₂O: C, 55.21; H, 6.00; N, 11.74. Found: C, 55.49; H, 6.13; N, 11.35.

### EXAMPLE 185

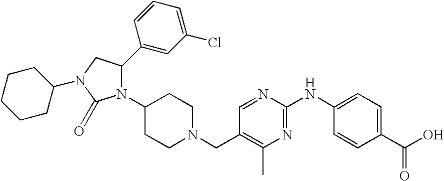

### COMPOUND 185: 4-(5-{4-[5-(3-Chloro-phenyl)-3-cyclohexyl-2-oxo-imidazolidin-1-yl]-piperidin-1-ylmethyl}-4-methyl-pyrimidin-2-ylamino)-benzoic acid

COMPOUND 185 was isolated as a brown solid (13 mg, 11% over 2 steps). ¹H NMR (CDCl₃) δ 1.02 (q, 1H, J=12.0 Hz), 1.15-1.45 (m, 7H), 1.63 (d, 2H, J=15.3 Hz), 1.76 (m, 3H), 1.99 (m, 2H), 2.10 (m, 1H), 2.47 (s, 3H), 2.76 (m, 1H), 2.94 (m, 1H), 3.03 (m, 1H), 3.36 (s, 2H), 3.65 (t, 1H, J=9.0 Hz), 3.77 (m, 2H), 4.55 (m, 1H), 7.15-7.26 (m, 3H), 7.33 (s, 1H, 7.87 (d, 2H, J=8.7 Hz), 8.09 (d, 2H, J=8.7 Hz), 8.19 (s, 1H), 8.99 (s, 1H); ES-MS m/z 603 (M+H).

Examples 186 to 204 were prepared following the scheme illustrated below. RCHO is as defined in the table and X is as defined in the individual examples.

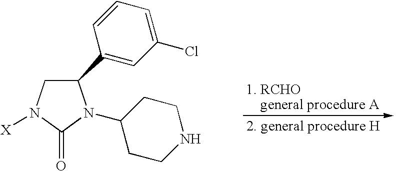

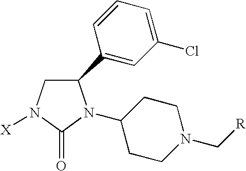

| Exam- | |
|---|---|
| ple | RCHO |
| 186 | 4-(5-formyl-4-methyl-pyridin-2-yloxy)-benzoic acid methyl ester |
| 187 | 4-(5-formyl-6-methyl-pyridin-2-yloxy)-benzoic acid methyl ester |
| 188 | 4-(4-formyl-3,5-dimethyl-pyrazol-1-ylmethyl)-benzoic acid |
| | methyl ester |
| 189 | 4-(5-formyl-pyridin-2-yloxy)-benzoic acid methyl ester |
| 190 | 4-(5-formyl-pyridin-2-yloxy)-benzoic acid methyl ester |
| 191 | 4-(5-formyl-6-methyl-pyridin-2-ylsulfanyl)-benzoic acid methyl |
| | ester |
| 192 | 4-(5-formyl-pyrimidin-2-yloxy)-benzoic acid methyl ester |
| 193 | 4-(5-formyl-6-methyl-pyridin-2-yloxy)-benzoic acid methyl ester |
| 194 | 4-(5-formyl-pyrimidin-2-ylsulfanyl)-benzoic acid methyl ester |
| 195 | 4-(5-formyl-6-methyl-pyridin-2-yloxy)-benzoic acid methyl ester |
| 196 | 4-(6-chloro-5-formyl-pyridin-2-yloxy)-benzoic acid methyl ester |
| 197 | 4-(5-formyl-6-methyl-pyridin-2-yloxy)-benzoic acid methyl ester |
| 198 | 4-(6-fluoro-5-formyl-pyridin-2-yloxy)-benzoic acid methyl ester |
| 199 | 4-(5-formyl-6-methyl-pyridin-2-ylsulfanyl)-benzoic acid methyl |
| | ester |
| 200 | 4-(5-formyl-pyrimidin-2-yloxy)-benzoic acid methyl ester |
| 201 | 4-(5-formyl-pyrimidin-2-yloxy)-benzoic acid methyl ester |
| 202 | 4-(5-formyl-pyrimidin-2-yloxy)-benzoic acid methyl ester |
| 203 | 4-(5-formyl-4,6-dimethyl-pyridin-2-yloxy)-benzoic acid methyl |
| | ester |
| 204 | 4-(5-formyl-4-methyl-pyrimidin-2-yloxy)-benzoic acid methyl |
| | ester (see EXAMPLE 93) |

### EXAMPLE 186

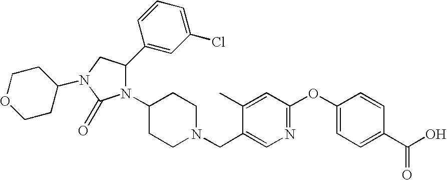

### COMPOUND 186: 4-(5-{4-[5-(3-Chloro-phenyl)-2-oxo-3-(tetrahydro-pyran-4-yl)-imidazolidin-1-yl]-piperidin-1-ylmethyl}-4-methyl-pyridin-2-yloxy)-benzoic acid

COMPOUND 186 was isolated as a white foam (0.086 g, 52% over 2 steps). ¹H NMR (CD₃OD) δ 1.60-1.88 (m, 7H), 2.20-2.35 (m, 1H), 2.43 (s, 3H), 2.85-2.93 (m, 2H), 3.11-3.17 (m, 1H), 3.35-3.63 (m, 5H), 3.80 (t, 1H, J=9.3 Hz), 3.88-4.00 (m, 3H), 4.12 (s, 2H), 4.74 (dd, 1H, J=9.3, 7.2 Hz), 6.97 (s, 1H), 7.18 (d, 2H, J=8.7 Hz), 7.33-7.42 (m, 4H), 8.06 (d, 2H, J=8.7 Hz), 8.09 (s, 1H); ¹³C NMR (CD₃OD) δ 19.74, 28.13, 28.83, 30.95, 31.24, 50.63, 51.45, 53.34, 55.75, 57.68, 68.28, 68.35, 114.77, 121.72, 123.03, 126.60, 128.30, 129.86, 132.01, 132.84, 136.03, 145.67, 151.86, 154.63, 159.34, 161.65, 165.18; ES-MS m/z 605 (M+H). Anal. Calcd. for C₃₃H₃₇ClN₄O₅.1.0CH₂Cl₂.1.7H₂O: C, 56.6; H, 5.93; N, 7.77; Cl, 14.76. Found: C, 56.65; H, 5.76; N, 7.84; Cl, 14.82.

### EXAMPLE 187

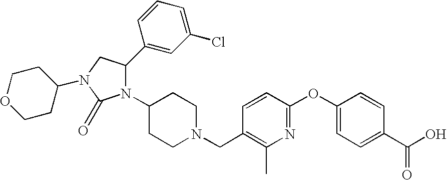

### COMPOUND 187: 4-(5-{4-[5-(3-Chloro-phenyl)-2-oxo-3-(tetrahydro-pyran-4-yl)-imidazolidin-1-yl]-piperidin-1-ylmethyl}-6-methyl-pyridin-2-yloxy)-benzoic acid

COMPOUND 187 was isolated as a white foam (0.022 g, 14% over 2 steps). ¹H NMR (CD₃OD) δ 1.61-1.87 (m, 7H), 2.21-2.33 (m, 1H), 2.44 (s, 3H), 2.75-2.86 (m, 2H), 3.13 (dd, 1H, J=8.7, 6.9 Hz), 3.23-3.37 (m, 2H), 3.44-3.51 (m, 2H), 3.56-3.64 (m, 1H), 3.80 (t, 1H, J=9.3 Hz), 3.88-4.00 (m, 3H), 4.05 (s, 2H), 4.75 (dd, 1H, J=9.3, 6.9 Hz), 6.86 (d, 1H, J=8.1 Hz), 7.16 (d, 2H, J=7.5 Hz), 7.33-7.40 (m, 3H), 7.42 (s, 1H), 7.79 (d, 1H, J=8.1 Hz), 8.05 (d, 2H, J=7.5 Hz); ¹³C NMR (CD₃OD) δ 22.17, 28.39, 29.14, 30.95, 31.25, 50.64, 51.78, 53.44, 53.56, 57.72, 58.01, 68.29, 68.36, 110.83, 121.44, 122.39, 126.60, 128.32, 129.87, 132.00, 132.91, 136.05, 145.10, 145.72, 159.37, 159.59, 161.71, 163.97; ES-MS m/z 605 (M+H). Anal. Calcd. for C₃₃H₃₇ClN₄O₅.0.6CH₂Cl₂.1.1H₂O: C, 59.71; H, 6.02; N, 8.29; Cl, 11.54. Found: C, 59.37; H, 5.63; N, 8.14; Cl, 11.64.

### EXAMPLE 188

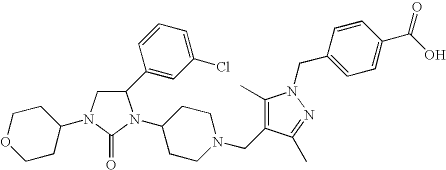

### COMPOUND 188: 4-(4-{4-[5-(3-Chloro-phenyl)-2-oxo-3-(tetrahydro-pyran-4-yl)-imidazolidin-1-yl]-piperidin-1-ylmethyl}-3,5-dimethyl-pyrazol-1-ylmethyl)-benzoic acid

To a solution of ethyl diacetoacetate (1.03 g, 5.98 mmol) in AcOH (20 mL) was added hydrazine hydrate (0.28 mL, 9.0 mmol) and the mixture was stirred at room temperature overnight. Standard aqueous work-up afforded the pyrazole (959 mg, 95%) which was subsequently reduced with LiAlH₄ (11.4 mL, 11.4 mmol) in THF (28 mL). Aqueous work-up followed by filtration afforded (3,5-dimethyl-1H-pyrazol-4-yl)-methanol as a colourless solid (441 mg, 61%).

A solution of the above alcohol (153 mg, 1.21 mmol), methyl 4-(bromomethyl)benzoate (278 mg, 1.21 mmol) and DIPEA (0.30 mL, 1.7 mmol) in CH₃CN (6.0 mL) was heated to reflux for 20 hours. Standard work-up and purification afforded the desired alcohol (237 mg, 71%) which was subsequently oxidized with MnO₂ (85%, 884 mg, 8.64 mmol) in CH₂Cl₂ (8.6 mL) at 40° C. Purification afforded 4-(4-formyl-3,5-dimethyl-pyrazol-1-ylmethyl)-benzoic acid methyl ester (119 mg, 51%).

COMPOUND 188 was isolated as a colourless foam (84 mg, 41% over 2 steps). ¹H NMR (DMSO-d₆) δ 1.51-1.68 (m, 7H), 2.12 (s, 3H), 2.16 (s, 3H), 2.18-2.30 (m, 1H), 2.52-2.80 (m, 1H), 2.94 (t, 1H, J=8.1 Hz), 3.15-3.41 (m, 6H), 3.66 (t, 1H, J=8.7 Hz), 3.74-3.86 (m, 5H), 4.69 (t, 1H, J=7.8 Hz), 5.27 (s, 2H), 7.17 (d, 2H, J=8.1 Hz), 7.32-7.44 (m, 4H), 7.87 (d, 2H, J=8.1 Hz); ¹³C NMR (DMSO-d₆) δ 10.23, 12.40, 29.58, 30.01, 48.09, 48.90, 49.53, 50.99, 51.95, 55.27, 55.39, 66.60, 66.69, 125.77, 127.11, 127.37, 128.36, 129.96, 130.25, 131.12, 133.61, 142.65, 145.29, 148.00, 159.48, 167.37; ES-MS m/z 606 (M+1). Anal. Calcd. for C₃₃H₄₀N₅ClO₄.1.0CH₂Cl₂: C, 59.09; H, 6.13; N, 10.13. Found: C, 58.95; H, 6.37; N, 10.23.

### EXAMPLE 189

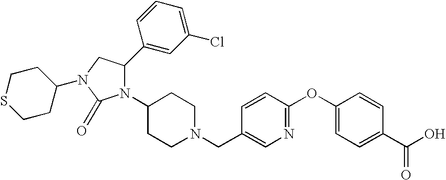

### COMPOUND 189: 4-(5-{4-[5-(3-Chloro-phenyl)-2-oxo-3-(tetrahydro-thiopyran-4-yl)-imidazolidin-1-yl]-piperidin-1-ylmethyl}-pyridin-2-yloxy)-benzoic acid

Following general procedure A: a solution of [2-amino-1-(3-chloro-phenyl)-ethyl]-carbamic acid tert-butyl ester (1.2 g, 4.4 mmol) and tetrahydro-4H-thiopyran-4-one (0.57 g, 4.8 mmol) were stirred in CH₂Cl₂ (25 mL) for 5 min, then NaBH(OAc)₃ (1.41 g, 6.6 mmol) was added and the mixture was stirred at room temperature under N₂ overnight. The crude [1-(3-chloro-phenyl)-2-(tetrahydro-thiopyran-4-ylamino)-ethyl]-carbamic acid tert-butyl ester was used in the next reaction without purification.

Following general procedure C: to a solution of the boc-protected amine (4.4 mmol) in CH₂Cl₂ (10 mL) was added TFA (4 mL). The reaction was stirred at room temperature for 2 h then concentrated to remove excess TFA. The crude 1-(3-chloro-phenyl)-N²-(tetrahydro-thiopyran-4-yl)-ethane-1,2-diamine was used in the next reaction without purification.

Following general procedure A: a solution of the above amine (4.4 mmol) and 1-boc-4-piperidone (0.97 g, 6.6 mmol) was stirred in CH₂Cl₂ (15 mL) for 5 min, then NaBH(OAc)₃ (1.41 g, 6.6 mmol) was added and the mixture was stirred at room temperature under N₂ overnight. The crude 4-[1-(3-chloro-phenyl)-2-(tetrahydro-thiopyran-4-ylamino)-ethylamino]-piperidine-1-carboxylic acid tert-butyl ester was used in the next reaction without purification.

To a solution of the above diamine (4.4 mmol) in CH₂Cl₂ (20 mL) was added DIPEA (1.54 mL, 8.8 mmol). The solution was cooled to 0° C., then triphosgene (0.65 g, 2.2 mmol) was added. After stirring at 0° C. for 1 h, saturated NaHCO₃ (20 mL) was added and the aqueous layer was extracted with CH₂Cl₂ (1×25 mL, 3×10 mL). The combined organic extracts were dried over Na₂SO₄ then concentrated. The crude product was purified by flash chromatography (CH₂Cl₂, 5% MeOH) to afford 4-[5-(3-chloro-phenyl)-2-oxo-3-(tetrahydro-thiopyran-4-yl)-imidazolidin-1-yl]-piperidine-1-carboxylic acid tert-butyl ester (1.56 g, 73% over 4 steps) as a white foam. ¹H NMR (CDCl₃) δ 0.97-1.19 (m, 1H), 1.39 (s, 9H), 1.42-1.50 (m, 1H), 1.57-1.80 (m, 4H), 1.99-2.09 (m, 2H), 2.48-2.74 (m, 4H), 2.75-2.91 (m, 2H), 3.04 (dd, 1H, J=6, 9 Hz), 3.61-3.78 (m, 3H), 3.88-4.20 (m, 2H), 4.52 (dd, 1H, J=6, 9 Hz), 7.15-7.21 (m, 1H), 7.25-7.29 (m, 3H).

To a solution of the boc-protected amine (451 mg, 0.94 mmol) in THF (3 mL) was added 6N HCl (3 mL). The reaction was stirred at 50° C. for 1 h, cooled, concentrated to remove THF, neutralized with 10N NaOH, and extracted with CH₂Cl₂ (3×20 mL). The organic extracts were dried over Na₂SO₄ and concentrated. The crude 4-(3-chloro-phenyl)-3-piperidin-4-yl-1-(tetrahydro-thiopyran-4-yl)-imidazolidin-2-one was used in the next reaction without purification (280 mg, 78%).

COMPOUND 189 was isolated as a white solid (232 mg, 52% over 2 steps) as a racemate. ¹H NMR (CDCl₃) δ 1.28-1.54 (m, 2H), 1.56-1.82 (m, 3H), 1.97-2.19 (m, 5H), 2.59-2.73 (m, 2H), 2.75-2.92 (m, 2H), 2.99-3.16 (m, 2H), 3.24-3.32 (m, 1H), 3.47-3.98 (m, 5H), 4.52-4.65 (m, 1H), 6.86 (d, 1H, J=9 Hz), 7.07-7.15 (m, 5H), 7.27 (s, 1H), 7.66 (d, 1H, J=9 Hz), 8.00 (d, 2H, J=6 Hz), 8.08 (s, 1H); ¹³C NMR (CDCl₃) δ 26.26, 27.52, 27.59, 28.47, 30.65, 30.72, 47.22, 49.81, 51.06, 54.19, 56.95, 110.77, 119.53, 123.86, 125.51, 127.60, 127.68, 129.30, 130.69, 133.82, 140.79, 143.58, 148.11, 156.46, 158.62, 162.14, 168.28; ES-MS m/z 607 (M+H).

### EXAMPLE 190

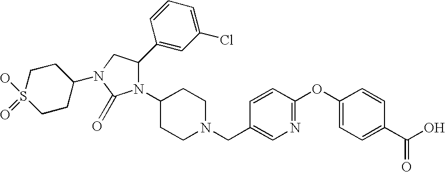

### COMPOUND 190: 4-(5-{4-[5-(3-Chloro-phenyl)-3-(1,1-dioxo-hexahydro-1λ⁶-thiopyran-4-yl)-2-oxo-imidazolidin-1-yl]-piperidin-1-ylmethyl}-pyridin-2-yloxy)-benzoic acid

A solution of 4-[5-(3-chloro-phenyl)-2-oxo-3-(tetrahydro-thiopyran-4-yl)-imidazolidin-1-yl]-piperidine-1-carboxylic acid tert-butyl ester (see EXAMPLE 189) (389 mg, 0.81 mmol) in MeOH (10 mL) was cooled to 0° C., and a solution of OXONE® (0.75 g, 1.2 mmol) in H₂O was added. The mixture was stirred at 0° C. for 15 min and at room temperature for another 30 min, then diluted with 1N NaOH (20 mL), which was extracted with CH₂Cl₂ (3×25 mL). The combined organic extracts were dried over Na₂SO₄ then concentrated. The crude 4-[5-(3-chloro-phenyl)-3-(1,1-dioxo-hexahydro-1λ⁶-thiopyran-4-yl)-2-oxo-imidazolidin-1-yl]-piperidine-1-carboxylic acid tert-butyl ester was used in the next reaction without purification.

To a solution of the boc-protected amine (0.81 mmol) in THF (3 mL) was added 6N HCl (3 mL). The reaction was stirred at 50° C. for 1 h, cooled, concentrated to remove THF, neutralized to pH 12 with 10N NaOH, and extracted with CH₂Cl₂ (3×10 mL). The organic extracts were dried over Na₂SO₄ and concentrated. The crude 4-(3-chloro-phenyl)-1-(1,1-dioxo-hexahydro-1λ⁶-thiopyran-4-yl)-3-piperidin-4-yl-imidazolidin-2-one was used in the next reaction without purification.

COMPOUND 190 was isolated as a white solid (167 mg, 32% over 4 steps) as a racemate. ¹H NMR (CDCl₃) δ 1.31-1.54 (m, 2H), 1.71-1.80 (m, 1H), 2.01-2.35 (m, 7H), 2.98-3.32 (m, 7H), 3.47-3.71 (m, 3H), 3.77-3.90 (m, 1H), 4.09-4.16 (m, 1H), 4.62 (dd, 1H, J=9, 6 Hz), 6.87 (d, 1H, J=9 Hz), 7.09-7.16 (m, 5H), 7.26 (s, 1H), 7.67 (d, 1H, J=9 Hz), 8.00 (d, 2H, J=6 Hz), 8.08 (s, 1H); ¹³C NMR (CDCl₃) δ 27.37, 29.07, 31.58, 36.66, 48.29, 48.75, 50.22, 50.81, 52.12, 55.30, 58.01, 111.95, 120,74, 124.92, 126.48, 128.96, 130.58, 131.83, 135.08, 142.06, 143.99, 149.33, 157.65, 159.52, 162.78, 163.34, 169.01; ES-MS m/z 661 (M+Na).

### EXAMPLE 191

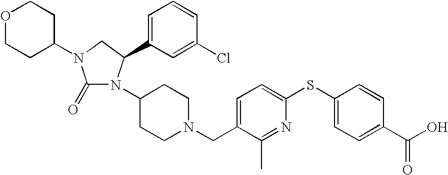

### COMPOUND 191: 4-(5-{4-[(R)-5-(3-Chloro-phenyl)-2-oxo-3-(tetrahydro-pyran-4-yl)-imidazolidin-1-yl]-piperidin-1-ylmethyl}-6-methyl-pyridin-2-ylsulfanyl)-benzoic acid

COMPOUND 191 was isolated as a pale yellow solid (39.5 mg, 65% over 2 steps). ¹H NMR (CDCl₃) δ 1.53-1.82 (m, 8H), 2.20-2.50 (m, 2H), 2.52 (s, 3H), 3.03 (t, 1H, J=7.5 Hz), 3.13 (br s, 1H), 3.30-3.52 (m, 3H), 3.65 (t, 1H, J=9.0 Hz), 3.76 (br s, 1H), 3.96-4.09 (m, 5H), 4.57 (br s, 1H), 6.37 (br s, 1H), 7.00-7.20 (m, 3H), 7.28 (br s, 1H), 7.49 (br s, 1H), 7.61 (d, 2H, J=7.8 Hz), 7.99 (d, 2H, J=7.8 Hz); ¹³C NMR (CDCl₃) δ 22.79, 27.03, 29.31, 30.29, 30.44, 48.63, 49.21, 50.83, 52.40, 55.54, 57.06, 67.49, 67.56, 119.23, 123.90, 125.28, 126.89, 129.07, 130.67, 131.37, 134.60, 135.00, 135.26, 140.45, 144.76, 157.82, 160.08, 170.22; ES-MS m/z 621 (M+H). Anal. Calcd. for C₃₃H₃₇ClN₄O₄S.1.1CH₂Cl₂: C, 57.31; H, 5.53; N, 7.84. Found: C, 57.32; H, 5.55; N, 7.91.

### EXAMPLE 192

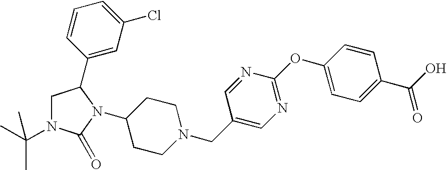

### COMPOUND 192: 4-(5-{4-[3-tert-Butyl-5-(3-chloro-phenyl)-2-oxo-imidazolidin-1-yl]-piperidin-1-ylmethyl}-pyrimidin-2-yloxy)-benzoic acid

Following general procedure F: to a solution of tert-butoxycarbonylamino-(3-chloro-phenyl)-acetic acid (400 mg, 1.4 mmol) in THF (6 mL) at 0° C. was added N-methylmorpholine (134 μL, 1.4 mmol). After allowing the reaction mixture to stir for 5 minutes at 0° C., IBCF (181 μL, 1.4 mmol) was added. After stirring for an additional 10 minutes at 0° C., t-butylamine (222 μL, 2.1 mmol) was added and the reaction mixture was left to stir at room temperature overnight. Standard work-up and recrystallization with EtOAc afforded [tert-butylcarbamoyl-(3-chloro-phenyl)-methyl]-carbamic acid tert-butyl ester as white crystals (265 mg, 55%).

Following general procedure C, the above substrate afforded 2-amino-N-tert-butyl-2-(3-chloro-phenyl)-acetamide as a light yellow solid (178 mg, 68%).

To a solution of the above amide (178 mg, 0.74 mmol) in THF (3.7 mL) was added BH₃.THF (1.0M in THF, 2.2 mL, 2.2 mmol). The reaction mixture was allowed to stir overnight at 60° C., cooled to room temperature and then quenched with MeOH (3 mL). The solvents were evacuated in vacuo and 6N HCl (3 mL) was added and the reaction mixture was heated to 80° C. for 1.5 hours. Basic work-up and purification afforded N²-tert-butyl-1-(3-chloro-phenyl)-ethane-1,2-diamine as a yellow oil (163 mg, 96%).

Following general procedure A: to a solution of the above amine (162 mg, 0.71 mmol) in dichloromethane (7 mL) was added N-boc-piperidone (157 mg, 0.79 mmol). After stirring at room temperature for 10 minutes, NaBH(OAc)₃ was added and the reaction mixture was allowed to stir for 3 days. Standard work-up and purification afforded 4-[2-tert-butylamino-1-(3-chloro-phenyl)-ethylamino]-piperidine-1-carboxylic acid tert-butyl ester as a pale yellow foam (133 mg, 46%).

Following general procedure K: to a solution of the above diamine (133 mg, 0.32 mmol) in dichloromethane (3.2 mL) at 0° C. under argon was added pyridine (52 μL, 0.65 mmol) followed by triphosgene (48 mg, 0.162 mmol) and the mixture was stirred at 0° C. for one hour. Standard work-up and purification afforded 4-[3-tert-butyl-5-(3-chloro-phenyl)-2-oxo-imidazolidin-1-yl]-piperidine-1-carboxylic acid tert-butyl ester (90 mg, 64%).

Following general procedure C, the above substrate (90 mg, 0.21 mmol) afforded 1-tert-butyl-4-(3-chloro-phenyl)-3-piperidin-4-yl-imidazolidin-2-one (69 mg, 100%).

COMPOUND 192 was isolated as a white solid (23 mg, 23% over 2 steps). ¹H NMR (CD₃OD) δ 1.22-1.51 (m, 1H), 1.35 (s, 9H), 1.47 (br d, 1H, J=25.2 Hz), 1.66 (br d, 1H, J=20.2 Hz), 1.90-2.03 (m, 1H), 2.14-2.31 (m, 2H), 2.91 (br d, 1H, J=11.4 Hz), 3.05 (br d, 1H, J=11.4 Hz), 3.13 (dd, 1H, J=9.0, 6.6 Hz), 3.55-3.62 (m, 3H), 3.79 (t, 1H, J=9.3 Hz), 4.63 (dd, 1H, J=8.8, 6.6 Hz), 7.25 (d, 2H, J=8.7 Hz), 7.30-7.35 (m, 4H), 7.40 (s, 1H), 8.07 (d, 2H, J=8.4 Hz), 8.52 (s, 2H); ES-MS m/z 564 (M+H). Anal. Calcd. for C₃₀H₃₄N₅ClO₄.0.8CH₂Cl₂: C, 58.53; H, 5.68; N, 11.08. Found: C, 58.85; H, 5.96; N, 10.83.

### EXAMPLE 193

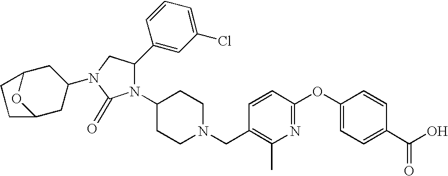

### COMPOUND 193: 4-(5-{4-[5-(3-Chloro-phenyl)-3-(8-oxa-bicyclo[3.2.1]oct-3-yl)-2-oxo-imidazolidin-1-yl]-piperidin-1-ylmethyl}-6-methyl-pyridin-2-yloxy)-benzoic acid

See EXAMPLE 178 for the preparation of 4-(3-chloro-phenyl)-1-(8-oxa-bicyclo[3.2.1]oct-3-yl)-3-piperidin-4-yl-imidazolidin-2-one. COMPOUND 193 was isolated as a pale yellow powder (ca. 9:1 cis-/trans-mixture) (25.0 mg, 35% over 2 steps). ¹H NMR (CDCl₃) δ 1.35-1.55 (m, 4H), 1.56-1.68 (m, 3H), 1.73-1.85 (m, 1H), 1.85-2.00 (m, 3H), 2.24-2.36 (m, 3H), 2.43 (s, 3H), 3.01-3.11 (m, 1H), 3.03 (dd, 1H, J=8.4, 6.0 Hz), 3.20-3.40 (m, 1H), 3.63 (t, 1H, J=9.0 Hz), 3.69-4.00 (m, 2H), 4.43 (m, 2H), 4.60 (m, 1H), 6.70 (d, 1H, J=5.7 Hz), 7.11-7.15 (m, 1H), 7.13 (d, 2H, J=8.7 Hz), 7.19 (s, 3H), 7.32 (s, 1H), 8.02 (d, 2H, J=8.7 Hz); ¹³C NMR (CDCl₃) δ 22.7, 27.1, 29.0, 30.1, 32.07, 32.11, 34.0, 34.1, 35.0, 43.5, 44.0, 50.5, 52.5, 52.7, 55.0, 57.2, 72.0, 72.1, 74.7, 109.8, 120.5, 125.4, 126.7, 127.7, 129.0, 130.8, 132.1, 135.3, 143.8, 145.0, 157.5, 158.4, 160.7, 162.5, 169.5; ES-MS m/z 631 (M+H). Anal. Calcd. for C₃₅H₃₉N₄O₅Cl.0.9CH₂Cl₂: C, 60.94; H, 5.81; N, 7.92. Found: C, 61.11; H, 5.97; N, 7.59.

### EXAMPLE 194

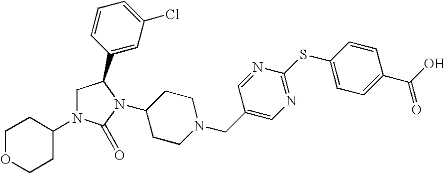

### COMPOUND 194: 4-(5-{4-[(R)-5-(3-Chloro-phenyl)-2-oxo-3-(tetrahydro-pyran-4-yl)-imidazolidin-1-yl]-piperidin-1-ylmethyl}-pyrimidin-2-ylsulfanyl)-benzoic acid

COMPOUND 194 was isolated as a white solid (484 mg, 70% over 2 steps). ¹H NMR (CDCl₃) δ 1.51 (br s, 2H), 1.64-1.71 (m, 3H), 1.77 (br d, 1H, J=13.2 Hz), 2.30 (br s, 2H), 2.39 (br s, 1H), 3.06 (dd, 2H, J=8.4, 6.3 Hz), 3.34 (br d, 1H, J=9.3 Hz), 3.41-3.52 (m, 3H), 3.64-3.75 (m, 3H), 3.98-4.02 (m, 4H), 4.60 (dd, 1H, J=9.0, 6.0 Hz), 7.08-7.16 (m, 3H), 7.29 (s, 1H), 7.65 (d, 2H, J=8.4 Hz), 8.01 (d, 2H, J=8.1 Hz), 8.48 (s, 2H); ES-MS m/z 608 (M+H). Anal. Calcd. for C₃₁H₃₄N₅ClO₄S.0.5CH₂Cl₂: C, 58.15; H, 5.42; N, 10.76. Found: C, 58.09; H, 5.62; N, 10.69.

### EXAMPLE 195

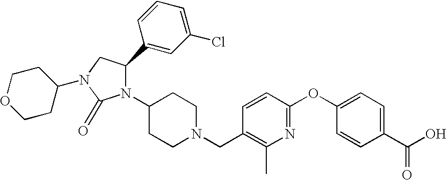

### COMPOUND 195: 4-(5-{4-[(R)-5-(3-Chloro-phenyl)-2-oxo-3-(tetrahydro-pyran-4-yl)-imidazolidin-1-yl]-piperidine-1-ylmethyl}-6-methyl-pyridin-2-yloxy)-benzoic acid

COMPOUND 195 was isolated as a white solid (368 mg, 88% over 2 steps). ¹H NMR (CD₃OD) δ 1.42 (dq, 1H, J=12.6, 3.9 Hz), 1.50-1.80 (m, 6H), 2.05 (dq, 1H, J=12.6, 3.6 Hz), 2.30 (m, 2H), 2.41 (s, 3H), 2.93 (d, 1H, J=11.7 Hz), 3.05 (d, 1H, J=11.7 Hz), 3.11 (m, 1H), 3.47 (t, 2H, J=11.7 Hz), 3.55 (m, 1H), 3.63 (s, 2H), 3.78 (t, 1H, J=9.3 Hz), 3.94 (m, 3H), 4.56 (m, 1H), 6.75 (d, 1H, J=8.1 Hz), 7.10 (d, 2H, J=8.4 Hz), 7.30-7.42 (m, 4H), 7.69 (d, 1H, J=8.4 Hz), 8.03 (d, 2H, J=8.7 Hz); ¹³C NMR (CD₃OD) δ 22.48, 28.01, 30.27, 30.31, 30.44, 48.64, 49.19, 51.49, 53.07 (2C), 55.68, 58.16, 67.59 (2C), 109.31, 120.14 (2C), 125.27, 126.94, 128.03, 128.97, 130.65, 132.10 (2C), 135.22, 142.94, 144.99, 157.38, 158.77, 160.24, 161.99, 169.83, 174.11; ES-MS m/z 605 (M+H). Anal. Calcd. for C₃₃H₃₇ClN₄O₅.0.6CH₂Cl₂: C, 61.51; H, 5.87; N, 8.54. Found: C, 61.56; H, 5.82; N, 8.44.

### EXAMPLE 196

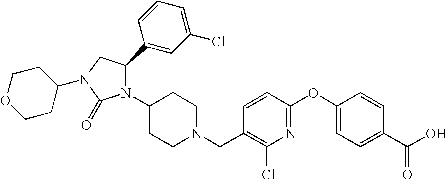

### COMPOUND 196: 4-(6-Chloro-5-{4-[(R)-5-(3-chloro-phenyl)-2-oxo-3-(tetrahydro-pyran-4-yl)-imidazolidin-1-yl]-piperidin-1-ylmethyl}-pyridin-2-yloxy)-benzoic acid

COMPOUND 196 was isolated as a yellow solid (70.8 mg, 37% over 2 steps). ¹H NMR (CDCl₃) δ 1.50 (br s, 2H), 1.66 (br s, 4H), 1.77 (d, 1H, J=13.2 Hz), 2.10-2.53 (m, 3H), 3.02-3.08 (m, 2H), 3.23 (br s, 1H), 3.44-3.52 (m, 2H), 3.67 (t, 1H, J=9.0 Hz), 3.70-3.91 (m, 3H), 3.98-4.10 (m, 3H), 4.59 (d, 1H, J=7.2 Hz), 6.75 (d, 1H, J=7.5 Hz), 7.12-7.9 (m, 5H), 7.30 (s, 1H), 7.88 (br s, 1H), 8.03 (d, 2H, J=5.7 Hz); ¹³C NMR (CDCl₃) δ 26.5, 28.6, 29.0, 29.1, 47.3, 47.8, 49.9, 51.5, 54.4, 55.8, 66.1, 66.2, 109.5, 119.4, 122.8, 123.9, 125.6, 127.7, 127.9, 129.4, 130.8, 133.9, 143.6, 148.2, 156.0, 158.9, 160.8, 168.4; ES-MS m/z 625 (M+H). Anal. Calcd. for C₃₂H₃₄N₄O₅Cl₂.1.4CH₂Cl₂: C, 53.89; H, 4.98; N, 7.53. Found: C, 53.70; H, 4.81; N, 7.50.

### EXAMPLE 197

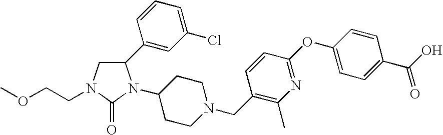

### COMPOUND 197: 4-(5-{4-[5-(3-Chloro-phenyl)-3-(2-methoxy-ethyl)-2-oxo-imidazolidin-1-yl]-piperidin-1-ylmethyl}-6-methyl-pyridin-2-yloxy)-benzoic acid

Following general procedure F: to a solution of tert-butoxycarbonylamino-(3-chloro-phenyl)-acetic acid (122 mg, 3.33 mmol) in THF (20 mL) cooled to 0° C. was added NMM (0.35 mL, 3.3 mmol) followed by IBCF (0.43 mL, 3.3 mmol). After stirring for 5 minutes, a solution of 2-methoxyethylamine (0.29 mL, 3.3 mmol) in THF (10 mL) was added. The mixture was stirred at room temperature overnight to afford the amide. Following general procedure C, the above substrate afforded crude 2-amino-2-(3-chloro-phenyl)-N-(2-methoxy-ethyl)-acetamide (899 mg). The above amide was treated with BH₃-THF (1.0 M in THF, 10 mL, 10 mmol) in THF (20 mL) and heated to 65° C. overnight. 6N HCl (10 mL) was added and the mixture heated to 85° C. for 2 hours. Standard work-up and purification afforded 1-(3-chloro-phenyl)-N²-(2-methoxy-ethyl)-ethane-1,2-diamine (427 mg, 57% over 3 steps).

Following general procedure A, the above amine (427 mg, 1.87 mmol) and 1-BOC-4-piperidone (372 mg, 1.87 mmol) afforded 4-[1-(3-chloro-phenyl)-2-(2-methoxy-ethylamino)-ethylamino]-piperidine-1-carboxylic acid tert-butyl ester after work-up and purification (588 mg, 76%).

Following general procedure K: to a solution of the above diamine (580 mg, 1.41 mmol) and pyridine (0.25 mL) in CH₂Cl₂ (14 mL) cooled to 0° C. was added triphosgene (208 mg, 0.700 mmol). The mixture was stirred at room temperature for 2 hours. Standard work-up afforded crude 4-[5-(3-chloro-phenyl)-3-(2-methoxy-ethyl)-2-oxo-imidazolidin-1-yl]-piperidine-1-carboxylic acid tert-butyl ester. Following general procedure C, the above carbamate afforded 4-(3-chloro-phenyl)-1-(2-methoxy-ethyl)-3-piperidin-4-yl-imidazolidin-2-one (324 mg, 68% over 2 steps).

COMPOUND 197 was isolated as a white solid (122 mg, 73% over 2 steps). ¹H NMR (CD₃OD) δ 1.75-1.89 (m, 3H), 2.30-2.43 (m, 1H), 2.46 (s, 3H), 2.97-3.02 (m, 2H), 3.23 (dd, 1H, J=9.3, 6.6 Hz), 3.32-3.52 (m, 9H), 3.62-3.75 (m, 1H), 3.86 (t, 1H, J=9.3 Hz), 4.22 (s, 2H), 4.76 (dd, 1H, J=9.3, 6.6 Hz), 6.86 (d, 1H, J=8.4 Hz), 7.16 (d, 2H, J=8.7 Hz), 7.30-7.35 (m, 3H), 7.45 (br s, 1H), 7.90 (d, 1H, J=8.4 Hz), 8.03 (d, 2H, J=8.7 Hz); ES-MS m/z 579 (M+1). Anal. Calcd. for C₃₁H₃₅ClN₄O₅.1.52H₂O.0.34CH₂Cl₂: C, 59.26; H, 6.14; N, 8.82. Found: C, 59.26; H, 6.14; N, 8.83.

### EXAMPLE 198

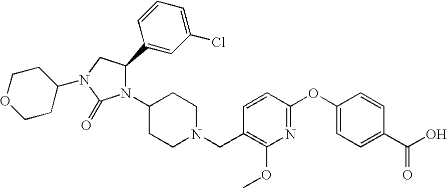

### COMPOUND 198: 4-(5-{4-[(R)-5-(3-Chloro-phenyl)-2-oxo-3-(tetrahydro-pyran-4-yl)-imidazolidin-1-yl]-piperidin-1-ylmethyl}-6-methoxy-pyridin-2-yloxy)-benzoic acid

COMPOUND 198 was isolated as an off-white solid (0.053 g, 56% over 2 steps). ¹H NMR (CD₃OD) δ 1.61-1.86 (m, 7H), 2.30-2.45 (m, 1H), 2.95-3.05 (m, 2H), 3.11-3.17 (m, 1H), 3.35-3.65 (m, 5H), 3.75 (s, 3H), 3.80 (t, 1H, J=9.3 Hz), 3.75-4.00 (m, 3H), 4.17 (s, 2H), 4.74 (dd, 1H, J=9.3, 7.2 Hz), 6.64 (d, 1H, J=8.1 Hz), 7.22-7.25 (m, 2H), 7.34-7.43 (m, 4H), 8.00 (d, 1H, J=8.1 Hz), 8.06-8.09 (m, 2H); ES-MS m/z 621 (M+H).

### EXAMPLE 199

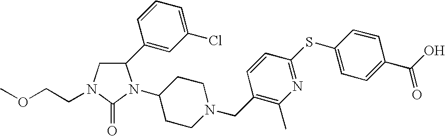

### COMPOUND 199: 4-(5-{4-[5-(3-Chloro-phenyl)-3-(2-methoxy-ethyl)-2-oxo-imidazolidin-1-yl]-piperidin-1-ylmethyl}-6-methyl-pyridin-2-ylsulfanyl)-benzoic acid

See EXAMPLE 197 for the preparation of 4-(3-chloro-phenyl)-1-(2-methoxy-ethyl)-3-piperidin-4-yl-imidazolidin-2-one. COMPOUND 199 was isolated as a white solid (115 mg, 93% over 2 steps). ¹H NMR (CDCl₃) δ 1.48-1.52 (m, 1H), 1.63-1.84 (m, 2H), 2.35-2.74 (m, 6H), 3.17-3.22 (m, 1H), 3.30 (s, 3H), 3.34-3.51 (m, 5H), 3.79 (t, 1H, J=9.3 Hz), 3.88-3.98 (m, 3H), 4.59 (dd, 1H, J=9.0, 6.6 Hz), 6.59 (d, 1H, J=7.8 Hz), 7.08-7.16 (m, 3H), 7.32 (s, 1H), 7.58 (d, 2H, J=8.1 Hz), 7.73 (br s, 1H), 7.99 (d, 2H, J=8.1 Hz); ¹³C NMR (CDCl₃) δ 23.17, 26.85, 29.00, 44.26, 50.67, 52.53, 54.12, 55.74, 56.96, 59.39, 71.78, 120.02, 125.83, 127.23, 129.28, 130.95, 131.56, 133.81, 135.12, 135.52, 135.91, 141.12, 145.17, 158.39, 161.07, 165.07, 169.88; ES-MS m/z 595 (M+1). Anal. Calcd. for C₃₁H₃₅ClN₄O₄S.1.07CH₂Cl₂.0.4H₂O: C, 55.79; H, 5.59; N, 8.01. Found: C, 55.80; H, 5.57; N, 7.98.

### EXAMPLE 200

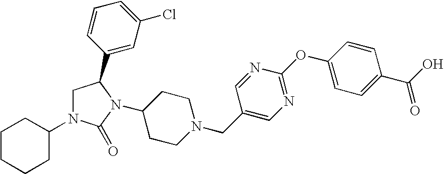

### COMPOUND 200: 4-(5-{4-[(R)-5-(3-Chloro-phenyl)-3-cyclohexyl-2-oxo-imidazolidin-1-yl]-piperidin-1-ylmethyl}-pyrimidin-2-yloxy)-benzoic acid

Following general procedure A: to a solution of [(R)-2-amino-1-(3-chloro-phenyl)-ethyl]-carbamic acid tert-butyl ester (656 mg, 2.42 mmol) in dichloromethane (25 mL) was added cyclohexanone (280 μL, 2.7 mmol) followed by NaBH(OAc)₃ (719 mg, 3.4 mmol) and the mixture was allowed to stir overnight. Standard work-up and purification afforded [(R)-1-(3-chloro-phenyl)-2-cyclohexylamino-ethyl]-carbamic acid tert-butyl ester (678 mg, 80%).

Following general procedure C, the Boc-protected amine above (678 mg, 1.92 mmol) afforded (R)-1-(3-chloro-phenyl)-N²-cyclohexyl-ethane-1,2-diamine (611 mg, quant).

Following general procedure A: to a solution of the amine above (611 mg, 2.42 mmol) in dichloromethane (25 mL) was added N-Boc piperidone (530 mg, 2.7 mmol) followed by NaBH(OAc)₃ and the mixture was allowed to stir overnight at room temperature. Standard work-up and purification afforded 4-[(R)-1-(3-chloro-phenyl)-2-cyclohexylamino-ethylamino]-piperidine-1-carboxylic acid tert-butyl ester (685 mg, 65%).

Following general procedure K: to a solution of the amine prepared above (685 mg, 1.57 mmol) in dichloromethane (16 mL) at 0° C. was added pyridine (254 μL. 3.14 mmol) followed by triphosgene (234 mg, 0.79 mmol) and the reaction mixture was allowed to stir at 0° C. for 45 minutes. Standard work-up and purification afforded 4-[(R)-5-(3-chloro-phenyl)-3-cyclohexyl-2-oxo-imidazolidin-1-yl]-piperidine-1-carboxylic acid tert-butyl ester (612 mg, 84%).

Following general procedure C, the cyclic urea prepared above (612 mg, 1.32 mmol) afforded (R)-2-(3-chloro-phenyl)-1-cyclohexyl-3-piperidin-4-yl-imidazolidin-2-one as a white solid (481 mg, quant).

COMPOUND 200 was isolated as a white solid (372 mg, 48% over 2 steps). ¹H NMR (CDCl₃) δ 0.94-1.06 (m, 1H), 1.18-1.74 (m, 12H), 2.17-2.45 (m, 3H), 3.01-3.06 (m, 2H), 3.29 (br d, 1H, J=9.9 Hz), 3.62-3.79 (m, 4H), 3.91 (br s, 1H), 4.55 (dd, 1H, J=9.0, 6.0 Hz), 7.07-7.20 (m, 5H), 7.28 (s, 1H), 8.02 (d, 2H, J=8.4 Hz), 8.54 (s, 2H), 10.20 (br s, 1H); ¹³C NMR (CDCl₃) δ 25.8, 29.6, 30.5, 30.6, 48.7, 50.8, 51.9, 52.6, 53.9, 55.7, 121.8, 125.4, 127.0, 128.9, 129.1, 130.7, 132.1, 135.2, 145.2, 156.7, 160.3, 161.9, 165.3, 169.2; ES-MS m/z 590 (M+H).

### EXAMPLE 201

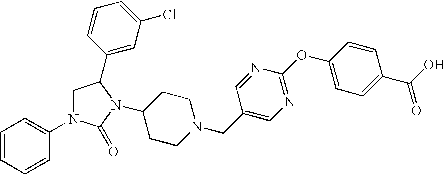

### COMPOUND 201: 4-(5-{4-[5-(3-Chloro-phenyl)-2-oxo-3-phenyl-imidazolidin-1-yl]-piperidin-1-ylmethyl}-pyrimidin-2-yloxy)-benzoic acid

See EXAMPLE 166 for the preparation of 4-(3-chloro-phenyl)-1-phenyl-3-piperidin-4-yl-imidazolidin-2-one. COMPOUND 201 was isolated as a white solid (16 mg, 27% over 2 steps). ¹H NMR (CDCl₃) δ 1.58 (br s, 2H), 1.89 (br d, 1H, J=10.5 Hz), 2.24-2.40 (m, 2H), 2.42-2.50 (m, 1H), 3.00-3.20 (m, 1H), 3.22-3.30 (m, 1H), 3.60-3.76 (m, 3H), 3.90-3.97 (br s, 1H), 4.20 (t, 1H, J=9.3 Hz), 4.77 (dd, 1H, J=9.3, 6.9 Hz), 7.06 (t, 1H, J=7.5 Hz), 7.14-7.24 (m, 5H), 7.31-7.36 (m, 3H), 7.52 (d, 2H, J=8.1 Hz), 8.05 (d, 2H, J=8.7 Hz), 8.56 (br s, 2H); ES-MS m/z 584 (M+H). Anal. Calcd. for C₃₂H₃₀N₅ClO₄.0.6CH₂Cl₂.1.2CH₃OH: C, 60.28; H, 5.39; N, 10.40. Found: C, 60.30; H, 5.26; N, 10.22.

### EXAMPLE 202

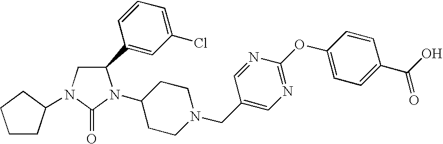

### COMPOUND 202: 4-(5-{4-[(R)-5-(3-Chloro-phenyl)-3-cyclopentyl-2-oxo-imidazolidin-1-yl]-piperidin-1-ylmethyl}-pyrimidin-2-yloxy)-benzoic acid

COMPOUND 202 was isolated as a white solid (352 mg, 67% over 2 steps). ¹H NMR (CD₃OD) δ 1.50-1.82 (m, 8H), 2.07-2.20 (m, 1H), 2.37-2.49 (m, 1H), 3.04-3.15 (m, 3H), 3.57-3.61 (m, 1H), 3.79-3.85 (m, 3H), 4.27-4.31 (m, 1H), 4.42-4.83 (m, 2H), 4.98-5.01 (m, 1H), 7.29-7.44 (m, 6H), 8.13 (d, 2H, J=8.7 Hz), 8.59 (s, 2H); ES-MS m/z 576 (M+1). Anal. Calcd. for C₃₁H₃₄ClN₅O₄.0.61H₂O.0.45CH₂Cl₂: C, 60.42; H, 5.82; N, 11.20. Found: C, 60.40; H, 5.79; N, 11.30.

### EXAMPLE 203

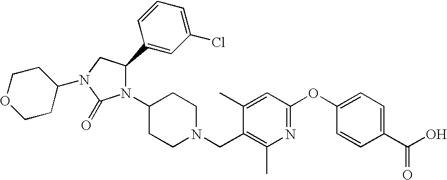

### COMPOUND 203: 4-(5-{4-[(R)-5-(3-Chloro-phenyl)-2-oxo-3-(tetrahydro-pyran-4-yl)-imidazolidin-1-yl]-piperidine-1-ylmethyl}-4,6-dimethyl-pyridin-2-yloxy)-benzoic acid

COMPOUND 203 was isolated as a white solid (54 mg, 25% over 2 steps). ¹H NMR (CD₃OD) δ 1.33 (dq, 1H, J=12.6, 3.9 Hz), 1.52 (d, 1H, J=12.0 Hz), 1.60-1.85 (m, 5H), 2.01 (dq, 1H, J=12.6, 3.6 Hz), 2.24 (m, 2H), 2.38 (s, 3H), 2.46 (s, 3H), 2.82 (d, 1H, J=11.7 Hz), 2.95 (d, 1H, J=11.1 Hz), 3.14 (m, 1H), 3.51 (t, 2H, J=11.4 Hz), 3.55 (m, 1H), 3.58 (s, 2H), 3.81 (t, 1H, J=9.0 Hz), 3.98 (m, 3H), 4.77 (m, 1H), 6.65 (s, 1H), 7.12 (d, 2H, J=8.7 Hz), 7.30-7.45 (m, 4H), 8.07 (d, 2H, J=8.7 Hz); ES-MS m/z 619 (M+H).

### EXAMPLE 204

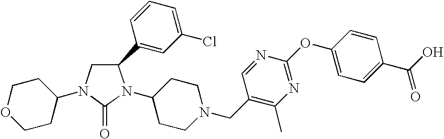

### COMPOUND 204: 4-(5-{4-[(R)-5-(3-Chloro-phenyl)-2-oxo-3-(tetrahydro-pyran-4-yl)-imidazolidin-1-yl]-piperidin-1-ylmethyl}-4-methyl-pyrimidin-2-yloxy)-benzoic acid

COMPOUND 204 was isolated as a white solid (66 mg, 46% over 2 steps). ¹H NMR (CDCl₃) δ 1.25-1.47 (m, 2H), 1.65-1.85 (m, 5H), 2.17-2.31 (m, 3H), 2.50 (s, 3H), 2.87-2.92 (m, 1H), 3.03-3.15 (m, 2H), 3.43-3.82 (m, 6H), 3.98-4.10 (m, 3H), 4.56-4.61 (m, 1H), 7.17-7.30 (m, 6H), 8.06 (d, 2H, J=8.4 Hz), 8.32 (br s, 1H); ¹³C NMR (CDCl₃) δ 22.53, 28.25, 30.31, 30.44, 48.62, 49.21, 51.51, 53.03, 55.79, 67.52, 67.57, 121.74, 125.26, 126.98, 127.89, 129.01, 130.68, 132.11, 135.26, 144.93, 157.18, 160.23, 160.99, 164.34, 169.33, 171.00; ES-MS m/z 606 (M+1). Anal. Calcd. for C₃₂H₃₆ClN₅O₅.0.78CH₂Cl₂.0.26CH₄O: C, 58.32; H, 5.72; N, 10.29. Found: C, 58.37; H, 5.61; N, 10.13.

Examples 205 to 208 were prepared following the scheme illustrated below. RCHO is as defined in the table and X is as defined in the individual examples.

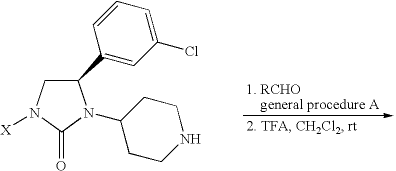

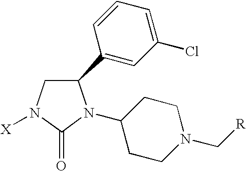

| Example | RCHO |
|---|---|
| 205 | [4-(5-formyl-6-methyl-pyridin-2-yloxy)-phenoxy]-acetic acid |
| | tert-butyl ester |
| 206 | [4-(5-formyl-6-methyl-pyridin-2-yloxy)-phenylsulfanyl]-acetic |
| | acid tert-butyl ester |
| 207 | [4-(5-formyl-pyridin-2-ylsulfanyl)-phenoxy]-acetic acid |
| | tert-butyl ester |
| 208 | 4-(6-ethyl-5-formyl-pyridin-2-yloxy)-benzoic acid tert-butyl |
| | ester |

### EXAMPLE 205

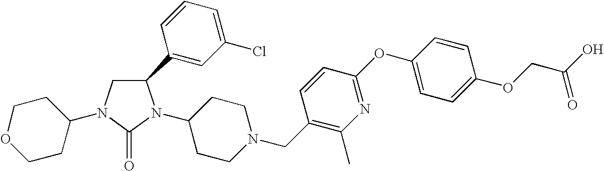

### COMPOUND 205: [4-(5-{4-[(R)-5-(3-Chloro-phenyl)-2-oxo-3-(tetrahydro-pyran-4-yl)-imidazolidin-1-yl]-piperidin-1-ylmethyl}-6-methyl-pyridin-2-yloxy)-phenoxy]-acetic acid

COMPOUND 205 was isolated as a white powder (106 mg, 59% over 2 steps). ¹H NMR (CDCl₃) δ 1.36-1.75 (m, 7H), 1.84 (d, 1H, J=12.3 Hz), 2.18-2.43 (m, 1H), 2.38 (s, 3H), 2.63 (m, 1H), 2.77 (t, 1H, J=12.0 Hz), 3.08 (dd, 1H, J=8.7, 6.3 Hz), 3.39 (d, 1H, J=11.7 Hz), 3.46-3.50 (m, 2H), 3.55 (d, 1H, J=10.8 Hz), 3.69 (t, 1H, J=9.0 Hz), 3.98-4.01 (m, 3H), 4.06 (s, 2H), 4.55 (s, 2H), 4.58 (dd, 1H, J=9, 6 Hz), 6.53 (d, 1H, J=8.4 Hz), 6.86 (d, 2H, J=9.0 Hz), 6.97 (d, 2H, J=9.0 Hz), 7.15-7.18 (m, 1H), 7.23-7.29 (m, 3H), 7.66 (d, 1H, J=8.4 Hz); ¹³C NMR (CDCl₃) δ 22.2, 25.6, 27.8, 29.9, 30.0, 48.3, 48.9, 49.1, 51.4, 51.8, 54.9, 55.9, 65.9, 67.0, 67.1, 108.4, 115.6, 117.2, 122.3, 125.0, 126.3, 128.9, 130.6, 135.1, 143.3, 144.1, 147.5, 155.2, 157.3, 159.5, 164.0, 171.6; ES-MS m/z 635 (M+H). Anal. Calcd. for C₃₄H₃₉N₄O₆Cl.2.4CH₂Cl₂: C, 52.44; H, 5.26; N, 6.68. Found: C, 52.31; H, 4.96; N, 6.63.

### EXAMPLE 206

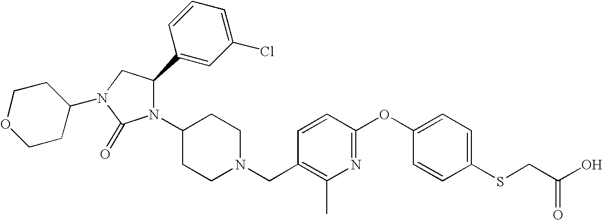

### COMPOUND 206: [4-(5-{4-[(R)-5-(3-Chloro-phenyl)-2-oxo-3-(tetrahydro-pyran-4-yl)-imidazolidin-1-yl]-piperidin-1-ylmethyl}-6-methyl-pyridin-2-yloxy)-phenylsulfanyl]-acetic acid

COMPOUND 206 was isolated as a white solid (40 mg, 43% over 2 steps). ¹H NMR (CD₃OD) δ 1.60-1.97 (m, 7H), 2.37 (dq, 1H, J=12.0, 3.6 Hz), 2.48 (s, 3H), 3.05 (m, 2H), 3.18 (m, 1H), 3.35-3.55 (m, 4H), 3.63 (m, 1H), 3.72 (s, 2H), 3.84 (t, 1H, J=9.0 Hz), 4.00 (m, 3H), 4.25 (s, 2H), 4.78 (m, 1H), 6.82 (d, 1H, J=8.4 Hz), 7.10 (d, 2H, J=8.7 Hz), 7.35-7.50 (m, 4H), 7.51 (d, 2H, J=8.7 Hz), 7.80 (d, 1H, J=8.7 Hz); ES-MS m/z 651 (M+H).

### EXAMPLE 207

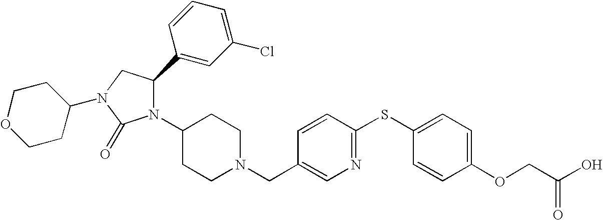

### COMPOUND 207: [4-(5-{4-[(R)-5-(3-Chloro-phenyl)-2-oxo-3-(tetrahydro-pyran-4-yl)-imidazolidin-1-yl]-piperidin-1-ylmethyl}-pyridin-2-ylsulfanyl)-phenoxy]-acetic acid

COMPOUND 207 was isolated as a white solid (35 mg, 55% over 2 steps). ¹H NMR (CD₃OD) δ 1.60-1.90 (m, 7H), 2.26 (dq, 1H, J=12.0, 3.6 Hz), 2.76 (q, 2H, J=12.0 Hz), 3.17 (m, 1H), 3.25 (d, 1H, J=14.4 Hz), 3.36 (m, 1H), 3.47 (t, 2H, J=14.1 Hz), 3.62 (tt, 1H, J=12.6, 3.6 Hz), 3.83 (t, 1H, J=9.3 Hz), 3.93 (m, 1H), 4.00 (m, 2H), 4.02 (s, 2H), 4.58 (s, 2H), 4.76 (m, 1H), 6.85 (d, 1H, J=8.4 Hz), 7.06 (d, 2H, J=8.7 Hz), 7.30-7.45 (m, 4H), 7.52 (d, 2H, J=8.7 Hz), 7.60 (dd, 1H, J=8.4, 2.1 Hz), 8.35 (s, 1H); ES-MS m/z 637 (M+H).

### EXAMPLE 208

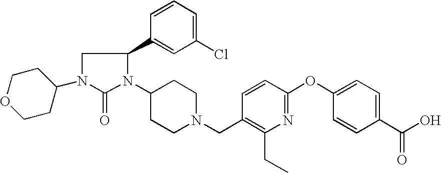

### COMPOUND 208: 4-(5-{4-[(R)-5-(3-Chloro-phenyl)-2-oxo-3-(tetrahydro-pyran-4-yl)-imidazolidin-1-yl]-piperidin-1-ylmethyl}-6-ethyl-pyridin-2-yloxy)-benzoic acid

COMPOUND 208 was isolated as a white powder (69.2 mg, 59% over 2 steps). ¹H NMR (CDCl₃) δ 1.10 (br s, 3H), 1.44 (br s, 2H), 1.64 (br s, 5H), 2.16-2.30 (m, 3H), 2.68 (br s, 2H), 2.93-3.14 (m, 3H), 3.46-4.00 (m, 7H), 4.58 (br s, 1H), 6.63 (br s, 1H), 7.16-7.29 (m, 6H), 7.70 (br s, 11H), 8.04 (br s, 2H); ¹³C NMR (CDCl₃) δ 11.91, 26.21, 28.49, 46.79, 47.35, 49.64, 51.10, 52.03, 53.78, 55.92, 65.71, 107.42, 118.54, 123.47, 125.11, 126.41, 127.15, 128.89, 130.16, 133.37, 141.06, 143.21, 156.71, 158.33, 159.98, 160.10, 168.82; ES-MS m/z 619 (M+H). Anal. Calcd. for C₃₃H₃₉ClN₄O₅.0.6CH₂Cl₂: C, 62.02; H, 6.05; N, 8.36. Found: C, 61.84; H, 6.05; N, 8.33.

### EXAMPLE 209

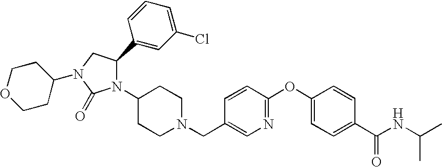

### COMPOUND 209: 4-(5-{4-[(R)-5-(3-Chloro-phenyl)-2-oxo-3-(tetrahydro-pyran-4-yl)-imidazolidin-1-yl]-piperidin-1-ylmethyl}-pyridin-2-yloxy)-N-isopropyl-benzamide

Following general procedure E: using 4-(5-{4-[(R)-5-(3-chloro-phenyl)-2-oxo-3-(tetrahydro-pyran-4-yl)-imidazolidin-1-yl]-piperidin-1-ylmethyl}-pyridin-2-yloxy)-benzoic acid (COMPOUND 168) (0.047 g, 0.080 mmol) and isopropylamine (0.0094 g, 0.16 mmol). Purification of the crude product by flash chromatography on silica gel (CH₂Cl₂/MeOH, 25:1 in v/v) gave COMPOUND 209 as a white foam (0.035 g, 67%). ¹H NMR (CDCl₃) δ 1.16-1.22 (m, 1H), 1.26 (d, 6H, J=7.2 Hz), 1.41-1.46 (m, 1H), 1.61-1.71 (m, 5H), 1.82-1.94 (m, 2H), 1.94-2.05 (m, 1H), 2.68-2.72 (m, 1H), 2.84-2.88 (m, 1H), 3.00 (dd, 1H, J=8.7, 6.3 Hz), 3.37 (s, 2H), 3.44-3.52 (m, 2H), 3.63-3.69 (m, 2H), 3.97-4.04 (m, 3H), 4.23-5.31 (m, 1H), 4.56 (dd, 1H, J=9.3, 6.3 Hz), 5.85 (d, 1H, J=7.5 Hz), 6.87 (d, 1H, J=8.4 Hz), 7.12-7.14 (m, 2H), 7.16-7.23 (m, 1H), 7.27-7.32 (m, 3H), 7.63 (dd, 1H, J=8.4, 2.4 Hz), 7.76-7.79 (m, 2H), 8.01 (d, 1H, J=2.4 Hz); ¹³C NMR (CDCl₃) δ 23.08, 29.24, 30.11, 30.26, 31.19, 42.08, 48.47, 48.92, 52.28, 53.04, 53.23, 55.69, 59.38, 67.36, 67.42, 111.77, 120.94, 124.90, 126.88, 128.71, 129.31, 130.44, 131.24, 134.96, 140.83, 145.17, 147.99, 157.07, 160.07, 162.53, 166.25; ES-MS m/z 632 (M+H); Anal. Calcd. for C₃₅H₄₂ClN₅O₄.0.2CH₂Cl₂: C, 65.13; H, 6.58; N, 10.79. Found: C, 65.03; H, 6.69; N, 10.65.

### EXAMPLE 210

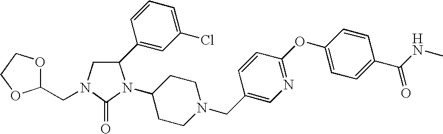

### COMPOUND 210: 4-(5-{4-[5-(3-Chloro-phenyl)-3-[1,3]dioxolan-2-ylmethyl-2-oxo-imidazolidin-1-yl]-piperidin-1-ylmethyl}-pyridin-2-yloxy)-N-methyl-benzamide

Following general procedure E: 4-(5-{4-[5-(3-chloro-phenyl)-3-[1,3]dioxolan-2-ylmethyl-2-oxo-imidazolidin-1-yl]-piperidin-1-ylmethyl}-pyridin-2-yloxy)-benzoic acid (COMPOUND 170) (35 mg, 0.059 mmol) and methylamine hydrochloride (8.0 mg, 0.12 mmol) afforded COMPOUND 210 as a white foam (28 mg, 78%). ¹H NMR (CDCl₃) δ 1.13-1.28 (m, 1H), 1.42-1.46 (m, 1H), 1.64-1.67 (m, 1H), 1.79-2.04 (m, 3H), 2.67-2.71 (m, 1H), 2.83-2.87 (m, 1H), 3.01 (d, 3H, J=4.8 Hz), 3.22 (dd, 1H, J=9.0, 6.0 Hz), 3.32-3.48 (m, 4H), 3.64-3.73 (m, 1H), 3.78-4.00 (m, 5H), 4.51 (dd, 1H, J=9.3, 6.0 Hz), 4.96 (t, 1H, J=4.2 Hz), 6.10-6.11 (m, 1H), 6.88 (d, 1H, J=8.4 Hz), 7.15 (d, 2H, J=8.4 Hz), 7.20-7.29 (m, 3H), 7.34 (br s, 1H), 7.63 (dd, 1H, J=8.4, 2.4 Hz), 7.78 (d, 2H, J=8.7 Hz), 8.01 (d, 1H, J=2.1 Hz); ¹³C NMR (CD₃Cl) δ 26.86, 29.10, 31.04, 46.58, 52.26, 52.87, 53.05, 53.98, 55.47, 59.18, 64.91, 65.06, 102.85, 111.67, 120.63, 124.84, 126.79, 128.37, 128.60, 129.25, 130.15, 130.65, 134.69, 140.66, 145.08, 147.75, 156.96, 160.58, 162.25, 167.58; ES-MS m/z 606 (M+1). Anal. Calcd. for C₃₂H₃₆N₅ClO₅.0.2CH₂Cl₂: C, 62.07; H, 5.89; N, 11.24. Found: C, 61.82; H, 5.93; N, 11.07.

### EXAMPLE 211

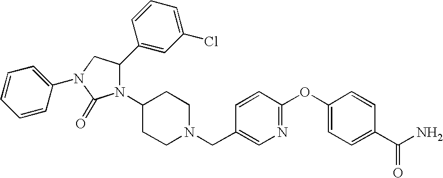

### COMPOUND 211: 4-(5-{4-[5-(3-Chloro-phenyl)-2-oxo-3-phenyl-imidazolidin-1-yl]-piperidin-1-ylmethyl}-pyridin-2-yloxy)-benzamide

To 4-(5-{4-[5-(3-chloro-phenyl)-2-oxo-3-phenyl-imidazolidin-1-yl]-piperidin-1-ylmethyl}-pyridin-2-yloxy)-benzonitrile (see EXAMPLE 166) (0.071 g, 0.13 mmol) in TFA (2.0 mL) was added 6 drops concentrated H₂SO₄. The mixture was heated at 90° C. for 3 days. After cooling, neutralization with 10 N NaOH was followed by extraction with CH₂Cl₂ (3×15 mL). The combined extract was dried over anhydrous Na₂SO₄. After filtration, the solvent was removed and the residue was purified by flash chromatography on silica gel (CH₂Cl₂/MeOH/NH₄OH, 200:10:1 v/v/v) to give COMPOUND 211 as a white foam (0.0090 g, 15%). ¹H NMR (CDCl₃) δ 1.25-1.32 (m, 1H), 1.48-1.60 (m, 1H), 1.72-1.81 (m, 1H), 1.91-2.10 (m, 2H), 2.73-2.77 (m, 1H), 2.88-2.92 (m, 1H), 3.41 (s, 2H), 3.54 (dd, 1H, J=9.0, 5.7 Hz), 3.75-3.83 (m, 1H), 4.18 (t, 1H, J=9.0 Hz), 4.70-4.75 (m, 1H), 5.61-5.96 (br m, 2H), 6.91 (d, 1H, J=8.4 Hz), 7.02-7.07 (m, 1H), 7.17-7.20 (m, 2H), 7.27-7.37 (m, 6H), 7.51-7.54 (m, 2H), 7.65-7.68 (m, 1H), 7.83-7.86 (m, 2H), 8.03 (d, 1H, J=2.1 Hz); ES-MS m/z 582 (M+H).

### EXAMPLE 212

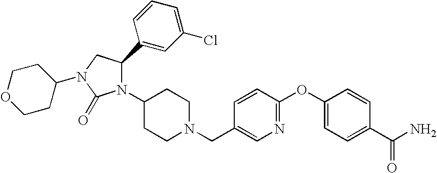

### COMPOUND 212: 4-(5-{4-[(R)-5-(3-Chloro-phenyl)-2-oxo-3-(tetrahydro-pyran-4-yl)-imidazolidin-1-yl]-piperidin-1-ylmethyl}-pyridin-2-yloxy)-benzamide

To 4-(5-{4-[(R)-5-(3-chloro-phenyl)-2-oxo-3-(tetrahydro-pyran-4-yl)-imidazolidin-1-yl]-piperidin-1-ylmethyl}-pyridin-2-yloxy)-benzonitrile (see EXAMPLE 168) (0.13 g, 0.23 mmol) in TFA (3.0 mL) was added 5 drops concentrated H₂SO₄. The mixture was heated at 85° C. for 16 h. After cooling, neutralization with 10 N NaOH was followed by extraction with CH₂Cl₂ (3×20 mL). The combined extract was dried over anhydrous Na₂SO₄. After filtration, the solvent was removed and the residue was purified by flash chromatography on silica gel (CH₂Cl₂/MeOH/NH₄OH, 500:25:1 v/v/v) to give COMPOUND 212 as a white foam (0.058 g, 44%). ¹H NMR (CDCl₃) δ 1.17-1.26 (m, 1H), 1.41-1.45 (m, 1H), 1.59-1.69 (m, 5H), 1.78-2.05 (m, 3H), 2.68-2.72 (m, 1H), 2.84-2.87 (m, 1H), 3.02 (dd, 1H, J=8.7, 6.6 Hz), 3.42 (s, 2H), 3.46-3.51 (m, 2H), 3.62-3.70 (m, 2H), 3.97-4.08 (m, 3H), 4.56 (dd, 1H, J=9.3, 6.6 Hz), 5.55 (br s, 1H), 6.00 (br s, 1H), 6.89 (d, 1H, J=8.4 Hz), 7.15-7.22 (m, 3H), 7.25-7.31 (m, 3H), 7.64 (dd, 1H, J=8.4, 2.4 Hz), 7.83-7.86 (m, 2H), 8.01 (d, 1H, J=2.4 Hz); NMR (CDCl₃) δ 29.24, 30.10, 30.26, 31.17, 48.47, 48.92, 52.28, 53.05, 53.23, 55.71, 59.38, 67.37, 67.43, 111.97, 120.89, 124.91, 126.89, 128.67, 129.38, 129.40, 130.45, 134.96, 140.92, 145.15, 147.98, 157.73, 160.09, 162.34, 168.84; ES-MS m/z 590 (M+H). Anal. Calcd. for C₃₂H₃₆ClN₅O₄.0.4CH₂Cl₂: C, 62.36; H, 5.94; N, 11.22. Found: C, 62.60; H, 6.03; N, 11.33.

### EXAMPLE 213 through 215

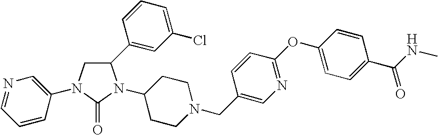

### COMPOUND 213: 4-(5-{4-[5-(3-Chloro-phenyl)-2-oxo-3-pyridin-3-yl-imidazolidin-1-yl]-piperidin-1-ylmethyl}-pyridin-2-yloxy)-N-methyl-benzamide

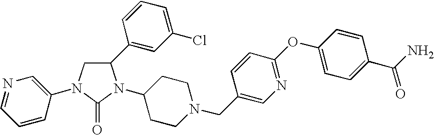

### COMPOUND 214: 4-(5-{4-[5-(3-Chloro-phenyl)-2-oxo-3-pyridin-3-yl-imidazolidin-1-yl]-piperidin-1-ylmethyl}-pyridin-2-yloxy)-benzamide

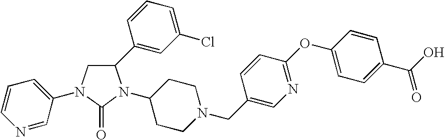

### COMPOUND 215: 4-(5-{4-[5-(3-Chloro-phenyl)-2-oxo-3-pyridin-3-yl-imidazolidin-1-yl]-piperidin-1-ylmethyl}-pyridin-2-yloxy)-benzoic acid

Following general procedure F: to a solution of tert-butoxycarbonylamino-(3-chloro-phenyl)-acetic acid (501 mg, 1.75 mmol) in THF (9 mL) at 0° C. was added NMM (177 mg, 1.75 mmol) in THF (1 mL) followed by IBCF (0.23 mL, 1.8 mmol) and the mixture stirred for 15 minutes. 2-Aminopyridine (330 mg, 3.51 mmol) was added and the mixture stirred at 0° C. for 30 min and at room temperature overnight. Standard work-up and purification gave [(3-chloro-phenyl)-(pyridin-3-ylcarbamoyl)-methyl]-carbamic acid tert-butyl ester (508 mg).

Following general procedure C with the above carbamate (508 mg) gave the crude intermediate. Reduction with BH₃.THF (1.0M in THF, 5.8 mL, 5.8 mmol) in THF (14 mL) at reflux followed by treatment with 6N HCl (6 mL) and subsequent basic work-up and purification afforded 1-(3-chloro-phenyl)-N²-pyridin-3-yl-ethane-1,2-diamine as a colourless oil (230 mg, 53% over 3 steps).

Following general procedure A, the above amine (230 mg, 0.928 mmol) and 1-BOC-4-piperidone (194 mg, 0.974 mmol) afforded the desired piperidine as a yellow solid (259 mg, 65%). Following general procedure K: to a solution of the piperidine (259 mg, 0.601 mmol) and Et₃N (0.17 mL, 1.2 mmol) in CH₂Cl₂ (11 mL) at 0° C. was added a solution of triphosgene (71 mg, 0.24 mmol) in CH₂Cl₂ (1 mL) and the mixture was stirred at room temperature for 2 hours. Standard work-up and purification afforded 4-[5-(3-chloro-phenyl)-2-oxo-3-pyridin-3-yl-imidazolidin-1-yl]-piperidine-1-carboxylic acid tert-butyl ester (114 mg, 41%). Following general procedure C, the above substrate (114 mg, 0.249 mmol) gave 4-(3-chloro-phenyl)-3-piperidin-4-yl-1-pyridin-3-yl-imidazolidin-2-one (86 mg, 97%).

Following general procedure G: a solution of the above amine (86 mg, 0.24 mmol), 4-(5-bromomethyl-pyridin-2-yloxy)-benzonitrile (84 mg, 0.29 mmol), DIPEA (0.067 mL, 0.38 mmol) and KI (catalytic) in CH₃CN (1.6 mL) was heated to 60° C. for 17 hours. Standard work-up and purification afforded 4-(5-{4-[5-(3-chloro-phenyl)-2-oxo-3-pyridin-3-yl-imidazolidin-1-yl]-piperidin-1-ylmethyl}-pyridin-2-yloxy)-benzonitrile (99 mg, 73%).

Following general procedure I, the above nitrile (67 mg, 0.12 mmol) afforded a mixture of COMPOUND 215 and the amide. Following general procedure E: a solution of the above mixture (40 mg), methylamine hydrochloride (9 mg, 0.1 mmol), EDCI (17 mg, 0.089 mmol), HOBT (12 mg, 0.089 mmol) and DIPEA (0.036 mL, 0.21 mmol) in DMF (1 mL) was stirred at room temperature overnight. Standard work-up and purification afforded COMPOUND 213 as a colourless foam (20 mg, 45% over 2 steps) and COMPOUND 214 as a yellow foam (13 mg, 13%).

COMPOUND 213: ¹H NMR (CDCl₃) δ 1.25-1.38(m, 1H), 1.49-1.54(m, 1H), 1.71-1.75 (m, 1H), 1.87-2.08 (m, 3H), 2.75 (d, 1H, J=11.7 Hz), 2.90 (d, 1H, J=9.9 Hz), 3.02 (d, 3H, J=4.8 Hz), 3.40 (s, 2H), 3.58 (dd, 1H, J=9.0, 5.4 Hz), 3.71-3.83 (m, 1H), 4.21 (t, 1H, J=9.3 Hz), 4.77 (dd, 1H, J=9.3, 5.4 Hz), 6.09-6.91 (m, 1H), 6.89 (d, 1H, J=8.4 Hz), 7.16 (d, 2H, J=8.4 Hz), 7.25-7.37 (m, 5H), 7.65 (dd, 1H, J=8.4, 2.1 Hz), 7.79 (d, 2H, J=8.4 Hz), 8.03 (d, 1H, J=1.8 Hz), 8.18 (d, 1H, J=8.4 Hz), 8.30 (d, 1H, J=4.2 Hz), 8.56 (d, 1H, J=2.4 Hz); ¹³C NMR (CDCl₃) δ 26.87, 29.23, 30.76, 51.47, 52.43, 52.77, 52.93, 54.72, 59.17, 111.71, 120.69, 123.46, 124.67, 124.76, 126.69, 128.58, 128.96, 129.12, 130.57, 130.68, 135.08, 136.68, 138.27, 140.67, 143.74, 144.06, 147.77, 156.94, 157.15, 162.31, 167.58; ES-MS m/z 519 (M+Na). Anal. Calcd. for C₃₃H₃₃N₆ClO₃.0.4CH₂Cl₂.0.2C₆H₁₄: C, 64.10; H, 5.69; N, 12.96. Found: C, 64.06; H, 5.62; N, 12.95.

COMPOUND 214: ¹H NMR (CDCl₃) δ 1.25-1.41 (m, 1H), 1.50-1.54 (m, 1H), 1.71-1.76 (m, 1H), 1.87-2.09 (m, 3H), 2.75 (d, 1H, J=12.0 Hz), 2.90 (d, 1H, J=11.4 Hz), 3.40 (s, 2H), 3.58 (dd, 1H, J=9.3, 5.4 Hz), 3.73-3.81 (m, 1H), 4.21 (t, 1H, J=9.3 Hz), 4.78 (dd, 1H, J=9.6, 5.4 Hz), 5.59-5.99 (br d, 2H), 6.91 (d, 1H, J=8.4 Hz), 7.18 (d, 2H, J=8.7 Hz), 7.25-7.37 (m, 5H), 7.66 (dd, 1H, J=8.4, 2.1 Hz), 7.84 (d, 2H, J=8.7 Hz), 8.03 (d, 1H, J=2.1 Hz), 8.16-8.19 (m, 1H), 8.30 (d, 1H, J=4.8 Hz), 8.56 (d, 1H, J=2.7 Hz); ¹³C NMR (CDCl₃) δ 29.24, 30.79, 51.47, 52.42, 52.78, 52.94, 54.71, 59.17, 111.81, 120.75, 123.44, 124.66, 124.75, 126.69, 128.97, 129.18, 130.57, 135.09, 138.27, 140.70, 143.79, 144.08, 147.49, 157.15, 157.54, 162.20, 168.49; ES-MS m/z 583 (M+1). Anal. Calcd. for C₃₂H₃₁N₆ClO₃.0.3CH₂Cl₂.0.7CH₄O: C, 62.82; H, 5.49; N, 13.32. Found: C, 62.93; H, 5.26; N, 13.04.

COMPOUND 215: ¹H NMR (CD₃OD) δ 1.62-1.89 (m, 3H), 2.18-2.31 (m, 1H), 2.45-2.58 (m, 2H), 3.13 (d, 1H, J=11.2 Hz), 3.23 (d, 1H, J=12.6 Hz), 3.64-3.73 (m, 2H), 3.86 (s, 2H), 4.30 (t, 1H, J=9.6 Hz), 4.97 (dd, 1H, J=9.3, 6.0 Hz), 7.05 (d, 1H, J=8.7 Hz), 7.17 (d, 2H, J=8.7 Hz), 7.35-7.42 (m, 4H), 7.50 (s, 1H), 7.87 (dd, 1H, J=8.4, 2.4 Hz), 7.99-8.07 (m, 3H), 8.13 (d, 1H, J=2.4 Hz), 8.21 (d, 1H, J=4.2 Hz), 8.84 (s, 1H); ¹³C NMR (CD₃OD) δ28.94, 29.92, 52.76, 52.91, 53.50, 53.65, 57.06, 58.94, 113.71, 121.89, 125.60, 126.00, 126.94, 127.03, 128.75, 130.11, 130.38, 132.39, 133.05, 136.42, 139.12, 140.30, 144.25, 145.52, 150.96, 159.10, 159.50, 165.05, 170.53; ES-MS m/z 584 (M+1). Anal. Calcd. for C₃₂H₃₀N₅ClO₄.0.4CH₂Cl₂.0.8CH₄O: C, 61.95; H, 5.32; N, 10.88. Found: C, 61.81; H, 5.24; N, 10.68.

### EXAMPLE 216

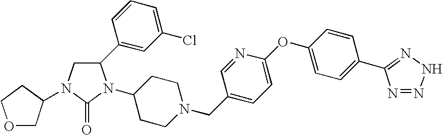

### COMPOUND 216: 4-(3-Chloro-phenyl)-1-(tetrahydro-furan-3-yl)-3-(1-{6-[4-(2H-tetrazol-5-yl)-phenoxy]-pyridin-3-ylmethyl}-piperidin-4-yl)-imidazolidin-2-one

A solution of 4-(5-{4-[5-(3-chloro-phenyl)-2-oxo-3-(tetrahydro-furan-3-yl)-imidazolidin-1-yl]-piperidin-1-ylmethyl}-pyridin-2-yloxy)-benzonitrile (see EXAMPLE 172) (60 mg, 0.11 mmol), NH₄Cl (24 mg, 0.44 mmol) and NaN₃ (21 mg, 0.33 mmol) in DMF (1 mL) was heated to 130° C. for 4 days. Standard work-up and purification afforded COMPOUND 216 as a yellow solid (22 mg, 33%). ¹H NMR (CD₃OD) δ 1.24-2.01 (m, 7H), 2.17-2.34 (m, 3H), 2.71-2.83 (m, 2H), 3.10-3.18 (m, 1H), 3.36-3.40 (m, 1H), 3.52-4.08 (m, 5H), 4.54-4.58 (m, 1H), 4.70-4.78 (m, 1H), 7.04 (d, 1H, J=8.4 Hz), 7.22 (d, 2H, J=8.4 Hz), 7.30-7.41 (m, 4H), 7.83-8.87 (m, 1H), 8.07 (d, 2H, J=8.4 Hz), 8.15 (br s, 1H); ¹³C NMR (CD₃OD) δ 11.79, 14.81, 24.43, 25.31, 25.94, 28.57, 29.15, 30.49, 30.74, 31.99, 35.17, 40.59, 48.57, 51.87, 52.39, 53.50, 54.76, 58.00, 58.10, 58.57, 68.10, 68.84, 71.71, 113.40, 123.04, 123.57, 126.86, 126.93, 127.35, 128.67, 129.82, 130.21, 132.30, 136.33, 144.41, 145.54, 145.72, 151.38, 156.50, 162.22, 165.93; ES-MS m/z 601 (M+1).

### EXAMPLE 217

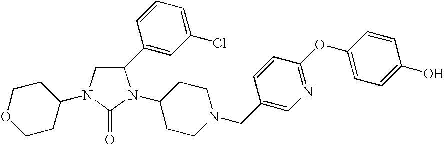

### COMPOUND 217: 4-(3-Chloro-phenyl)-3-{1-[6-(4-hydroxy-phenoxy)-pyridin-3-ylmethyl]-piperidin-4-yl}-1-(tetrahydro-pyran-4-yl)-imidazolidin-2-one

A mixture of 2-bromo-5-methylpyridine (2.38 g, 13.8 mmol), 4-methoxyphenol (430 mg, 3.46 mmol) and K₂CO₃ (479 mg, 3.47 mmol) was heated at 200° C. for 2.3 hours. Aqueous work-up and purification afforded 2-(4-methoxy-phenoxy)-5-methyl-pyridine (480 mg, 64%).

To the above methyl ether (237 mg, 1.10 mmol) in CH₂Cl₂ (2.2 mL) at 0° C. was added BBr₃ (1.0M in CH₂Cl₂, 3.30 mL, 3.30 mmol) and the mixture stirred at 0° C. for 1.5 hours. Treatment with 6N HCl followed by basic work-up afforded 4-(5-methyl-pyridin-2-yloxy)-phenol (95 mg, 43%).

To the above alcohol (95 mg, 0.47 mmol) and Et₃N (0.092 mL, 0.66 mmol) in CH₂Cl₂ (4.7 mL) was added AcCl (0.040 mL, 0.56 mmol) and the mixture stirred at room temperature for 1 hour. Aqueous work-up afforded acetic acid 4-(5-methyl-pyridin-2-yloxy)-phenyl ester (107 mg, 93%).

The above acetate (107 mg, 0.440 mmol), NBS (94 mg, 0.53 mmol) and (BzO)₂ (16 mg, 0.066 mmol) in CCl₄ (1.5 mL) were heated to reflux for 3.5 hours. Filtration and purification afforded acetic acid 4-(5-bromomethyl-pyridin-2-yloxy)-phenyl ester (63 mg, 44%).

Following general procedure G: a solution of 4-(3-chloro-phenyl)-3-piperidin-4-yl-1-(tetrahydro-pyran-4-yl)-imidazolidin-2-one (58 mg, 0.16 mmol), the above bromide (62 mg, 0.19 mmol) and DIPEA (0.044 mL, 0.25 mmol) in CH₃CN (1 mL) was heated to 60° C. for 18 hours. Standard work-up and purification afforded acetic acid 4-(5-{4-[5-(3-chloro-phenyl)-2-oxo-3-(tetrahydro-pyran-4-yl)-imidazolidin-1-yl]-piperidin-1-ylmethyl}-pyridin-2-yloxy)-phenyl ester (53 mg, 55%).

To the above acetate (52 mg, 0.086 mmol) in MeOH (3 mL) was added 1N NaOH (0.4 mL) and the mixture was stirred at room temperature for 10 minutes. Standard work-up and purification afforded COMPOUND 217 as a colourless foam (42 mg, 88%). ¹H NMR (CDCl₃) δ 1.09-1.23 (m, 1H), 1.42 (d, 1H, J=12.3 Hz), 1.53-1.73 (m, 5H), 1.80-1.95 (m, 2H), 2.02-2.09 (m, 1H), 2.73 (d, 1H, J=10.5 Hz), 2.92 (d, 1H, J=11.4 Hz), 3.01 (dd, 1H, J=8.7, 6.6 Hz), 3.30-3.53 (m, 4H), 3.61-3.78 (m, 2H), 3.97-4.09 (m, 3H), 4.52 (dd, 1H, J=9.0, 6.3 Hz), 6.63 (d, 2H, J=9.0 Hz), 6.68 (d, 1H, J=8.4 Hz), 6.89 (d, 2H, J=8.7 Hz), 7.07-7.26 (m, 4H), 7.55 (dd, 1H, J=8.4, 2.1 Hz), 8.00 (d, 1H, J=1.8 Hz); ¹³C NMR (CDCl₃) δ 28.46, 29.91, 30.05, 30.73, 48.27, 48.80, 51.79, 52.76, 52.85, 55.67, 59.21, 67.14, 67.19, 110.11, 116.56, 122.33, 124.72, 126.52, 126.85, 128.48, 130.19, 134.77, 141.01, 144.68, 146.42, 148.24, 153.85, 159.97, 164.11; ES-MS m/z 563 (M+1). Anal. Calcd. for C₃₁H₃₅N₄ClO₄.0.4CH₂Cl₂: C, 63.17; H, 6.04; N, 9.38. Found: C, 63.19; H, 6.10; N, 9.17.

### EXAMPLE 218

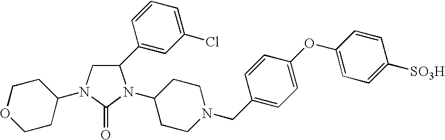

### COMPOUND 218: 4-(5-{4-[5-(3-Chloro-phenyl)-2-oxo-3-(tetrahydro-pyran-4-yl)-imidazolidin-1-yl]-piperidin-1-ylmethyl}-pyridin-2-yloxy)-benzenesulfonic acid

To a solution of 4-fluorobenzaldehyde (348 mg, 2.80 mmol) in DMA (4 mL) was added phenol (409 mg, 4.35 mmol) and K₂CO₃ (780 mg, 5.65 mmol) and the reaction stirred at 130° C. for 5.5 h. The mixture was cooled and diluted with brine (30 mL) and EtOAc (30 mL). The organic layer was washed with brine (2×20 mL), dried (Na₂SO₄), concentrated and purified by column chromatography on silica gel (EtOAc/Hexanes, 1:4) to afford the desired ether (582 mg) as a yellow oil. ¹H NMR (CDCl₃) δ 7.05-7.11 (m, 3H), 7.23-7.26 (m, 2H), 7.42 (t, 2H, J=7.5 Hz), 7.85 (d, 2H, J=9 Hz), 9.93 (s, 1H).

To a solution of 4-phenoxy-benzaldehyde (502 mg, 2.53 mmol) in MeOH (13 mL) was added NaBH₄ (96 mg, 2.5 mmol) and the mixture was stirred at room temperature for 1 hour. Standard aqueous work-up and purification afforded the alcohol (350 mg, 69%).

To a solution of the alcohol (173 mg, 0.864 mmol) in CH₂Cl₂ (4.3 mL) at 0° C. was added PBr₃ (0.081 mL, 0.86 mmol) and the mixture stirred at 0° C. for 20 minutes. Standard work-up afforded 1-bromomethyl-4-phenoxy-benzene (184 mg, 81%).

A solution of the bromide (184 mg, 0.699 mmol), 4-(3-chloro-phenyl)-3-piperidin-4-yl-1-(tetrahydro-pyran-4-yl)-imidazolidin-2-one (247 mg, 0.679 mmol) and DIPEA (0.17 mL, 0.98 mmol) in CH₃CN (4.5 mL) was heated at 60° C. for 19 hours. Standard work up and purification afforded 4-(3-chloro-phenyl)-3-[1-(6-phenoxy-pyridin-3-ylmethyl)-piperidin-4-yl]-1-(tetrahydro-pyran-4-yl)-imidazolidin-2-one as a colourless foam (197 mg, 53%).

To solution of the above substrate (183 mg, 0.335 mmol) in CH₂Cl₂ (0.3 mL) at 0° C. was added a solution of ClSO₃H (0.023 mL, 0.34 mmol) in CH₂Cl₂ (0.3 mL) (J. Med. Chem., 46, 2436-2445 (2003)). The mixture was stirred at 0° C. for 5 minutes and at room temperature for 3 hours. A second aliquot of ClSO₃H (0.046 mL, 0.69 mmol) was added at 0° C. and the mixture stirred at room temperature overnight. Standard work-up and purification afforded COMPOUND 218 as a colourless solid (77 mg, 37%). ¹H NMR (DMSO-d₆) δ 1.51-1.74 (m, 7H), 2.12-2.25 (m, 1H), 2.87-2.97 (m, 3H), 3.23-3.32 (m, 4H), 3.40-3.48 (m, 1H), 3.66 (t, 1H, J=9.0 Hz), 3.74-3.84 (m, 3H), 4.10-4.12 (m, 2H), 4.69 (t, 1H, J=8.4 Hz), 6.95 (d, 2H, J=8.1 Hz), 7.03 (d, 2H, J=8.1 Hz), 7.32-7.42 (m, 6H), 7.61 (d, 2H, J=8.1 Hz), 9.27 (br s, 1H); ¹³C NMR (DMSO-d₆) δ 26.51, 27.20, 29.52, 30.06, 48.04, 48.89, 49.65, 51.25, 55.59, 58.73, 66.58, 66.69, 118.55, 118.66, 124.70, 125.80, 127.19, 127.99, 128.47, 131.20, 133.64, 144.69, 144.96, 156.13, 158.12, 159.41; ES-MS m/z 626 (M+1). Anal. Calcd. for C₃₂H₃₆N₃ClSO₆.0.9CH₂Cl₂: C, 56.24; H, 5.42; N, 5.98. Found: C, 55.98; H, 5.55; N, 6.11.

Examples 219 to 222 were prepared following the scheme illustrated below. RCHO is as defined in the table and X is as defined in the individual examples.

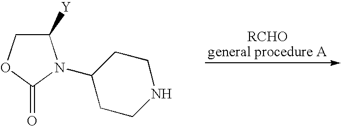

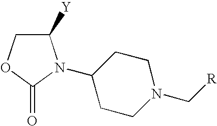

| Exam- | |
|---|---|
| ple | RCHO |
| 219 | N-cyclopropyl-4-(5-formyl-6-methyl-pyridin-2-yloxy)- |
| | benzamide |
| 220 | 6-(4-methoxy-phenylsulfanyl)-2-methyl-pyridine-3-carbaldehyde |
| 221 | 6-(4-methoxy-phenoxy)-2-methyl-pyridine-3-carbaldehyde |
| 222 | 6-(3-tert-butyl-4-hydroxy-phenoxy)-2-methyl-pyridine-3- |
| | carbaldehyde |

### EXAMPLE 219

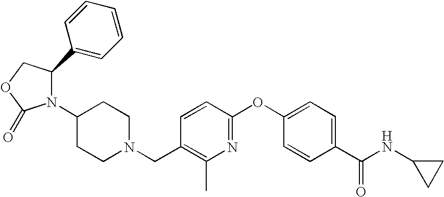

### COMPOUND 219: N-Cyclopropyl-4-{6-methyl-5-[4-((R)-2-oxo-4-phenyl-oxazolidin-3-yl)-piperidin-1-ylmethyl]-pyridin-2-yloxy}-benzamide

COMPOUND 219 was isolated as a white solid (14 mg, 14%). ¹H NMR (CDCl₃) δ 0.61 (m, 2H), 0.88 (q, 2H, J=6.6 Hz), 1.25 (m, 1H), 1.50 (d, 1H, J=12.0 Hz), 1.63 (m, 1H), 1.70-2.05 (m, 3H), 2.37 (s, 3H), 2.67 (d, 1H, J=11.1 Hz), 2.84 (d, 1H, J=11.4 Hz), 2.90 (m, 1H), 3.31 (s, 2H), 3.60 (m, 1H), 4.08 (m, 1H), 4.58 (t, 1H, J=9.0 Hz), 4.80 (m, 1H), 6.18 (s, 1H), 6.61 (d, 1H, J=8.1 Hz), 7.12 (d, 2H, J=8.7 Hz), 7.30-7.45 (m, 5H), 7.48 (d, 1H, J=8.1 Hz), 7.73 (d, 2H, J=8.7 Hz); ES-MS m/z 527 (M+H).

### EXAMPLE 220

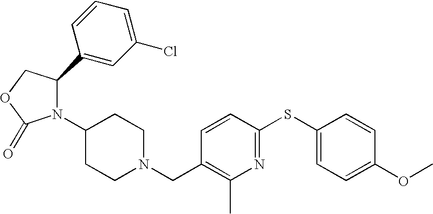

### COMPOUND 220: (R)-4-(3-Chloro-phenyl)-3-{1-[6-(4-methoxy-phenylsulfanyl)-2-methyl-pyridin-3-ylmethyl]-piperidin-4-yl}-oxazolidin-2-one

COMPOUND 220 was isolated as a white powder (162 mg, 58%). ¹H NMR (CDCl₃) δ 1.13-1.28 (m, 1H), 1.46 (d, 1H, J=12.0 Hz), 1.70 (d, 1H, J=9.9 Hz), 1.80-2.03 (m, 3H), 2.44 (s, 3H), 2.64 (d, 1H, J=9.9 Hz), 2.80 (d, 1H, J=10.8 Hz), 3.26 (s, 2H), 3.61 (m, 1H), 3.84 (s, 3H), 4.04 (dd, 1H, J=8.7, 5.4 Hz), 4.56 (t, 1H, J=8.7 Hz), 4.74 (dd, 1H, J=8.7, 5.4 Hz), 6.45 (d, 1H, J=8.1 Hz), 6.94 (d, 2H, J=8.7 Hz), 7.17-7.33 (m, 5H), 7.51 (d, 2H, J=8.7 Hz); ¹³C NMR (CDCl₃) 622.4, 29.7, 31.0, 53.1, 53.2, 53.5, 55.8, 58.2, 59.6, 70.6, 115.6, 117.7, 121.8, 125.2, 127.2, 127.9, 129.5, 131.0, 135.5, 137.6, 138.4, 143.2, 158.1, 158.3, 160.8, 161.0; ES-MS m/z 524 (M+H). Anal. Calcd. for C₂₈H₃₀N₃O₃SCl.0.3CH₂Cl₂: C, 61.85; H, 5.61; N, 7.65. Found: C, 62.02; H, 5.66; N, 7.47.

### EXAMPLE 221

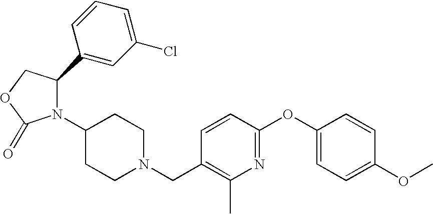

### COMPOUND 221: (R)-4-(3-Chloro-phenyl)-3-{1-[6-(4-methoxy-phenoxy)-2-methyl-pyridin-3-ylmethyl]-piperidin-4-yl}-oxazolidin-2-one

COMPOUND 221 was isolated as a white powder (178 mg, 66%). ¹H NMR (CDCl₃) δ 1.13-1.28 (m, 1H), 1.49 (d, 1H, J=11.7 Hz), 1.72 (d, 1H, J=9.9 Hz), 1.84-2.05 (m, 3H), 2.40 (s, 3H), 2.69 (d, 1H, J=11.4 Hz), 2.85 (d, 1H, J=11.4 Hz), 3.31 (s, 2H), 3.63 (m, 1H), 3.81 (s, 3H), 4.05 (dd, 1H, J=8.7, 5.4 Hz), 4.57 (t, 1H, J=8.7 Hz), 4.75 (dd, 1H, J=9.0, 5.1 Hz), 6.43 (d, 1H, J=8.1 Hz), 6.90 (d, 2H, J=9.3 Hz), 7.05 (d, 2H, J=9.3 Hz), 7.19-7.23 (m, 1H), 7.28-7.34 (m, 3H), 7.39 (d, 1H, J=8.1 Hz); ¹³C NMR (CDCl₃) δ 21.9, 29.3, 30.6, 52.7, 52.8, 53.1, 55.6, 57.8, 58.9, 70.2, 106.4, 114.7, 122.0, 124.7, 126.0, 126.8, 129.1, 130.6, 135.1, 140.8, 142.8, 147.9, 156.4, 156.5, 157.9, 162.6; ES-MS m/z 508 (M+H). Anal. Calcd. for C₂₈H₃₀N₃O₄Cl.0.1CH₂Cl₂: C, 65.34; H, 5.89; N, 8.14. Found: C, 65.21; H, 5.90; N, 8.06.

### EXAMPLE 222

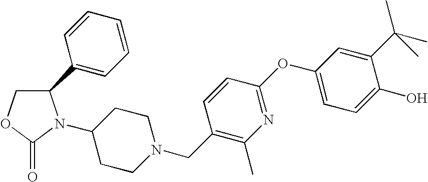

### COMPOUND 222: (R)-3-{1-[6-(3-tert-Butyl-4-hydroxy-phenoxy)-2-methyl -pyridin-3-ylmethyl]-piperidin-4-yl}-4-phenyl-oxazolidin-2-one

A mixture of tert-butylhydroquinone (410 mg, 2.46 mmol), 6-bromo-2-methyl-pyridine-3-carbaldehyde (497 mg, 2.46 mmol) and K₂CO₃ (340 mg, 2.46 mmol) in DMF (2.5 mL) was heated at 100° C. for 2 hours. Standard work-up and purification afforded 6-(3-tert-butyl-4-hydroxy-phenoxy)-2-methyl-pyridine-3-carbaldehyde (168 mg, 24%).

COMPOUND 222 was isolated as a white solid (165 mg, 57%). ¹H NMR (CDCl₃) δ 1.17-1.26 (m, 1H), 1.39 (s, 9H), 1.43-1.49 (m, 1H), 1.70-1.74 (m, 1H), 1.85-2.05 (m, 3H), 2.40 (s, 3H), 2.64-2.68 (m, 1H), 2.83-2.88 (m, 1H), 3.25-3.35 (m, 2H), 3.57-3.65 (m, 1H), 4.09 (dd, 1H, J=8.4, 5.7 Hz), 4.57 (t, 1H, J=8.7 Hz), 4.78 (dd, 1H, J=9.0, 5.7 Hz), 5.06 (br s, 1H), 6.37 (d, 1H, J=8.1 Hz), 6.60 (d, 1H, J=8.4 Hz), 6.79 (dd, 1H, J=8.1, 2.4 Hz), 7.05 (d, 1H, J=3.0 Hz), 7.29-7.39 (m, 6H); ES-MS m/z 516 (M+1).

Examples 223 to 233 were prepared following the scheme illustrated below. RCHO is as defined in the table and Y is as defined in the individual examples.

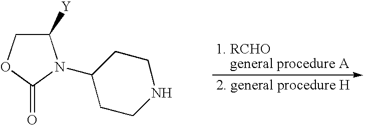

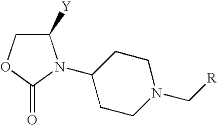

| Exam- | |
|---|---|
| ple | RCHO |
| 223 | 4-(5-formyl-6-methyl-pyridin-2-ylsulfanyl)-benzoic acid methyl |
| | ester |
| 224 | 4-(5-formyl-6-methyl-pyridin-2-yloxy)-benzoic acid methyl ester |
| 225 | 4-(5-formyl-6-methyl-pyridin-2-yloxy)-benzoic acid methyl ester |
| 226 | 4-(5-formyl-6-methyl-pyridin-2-ylsulfanyl)-benzoic acid methyl |
| | ester |
| 227 | 4-(5-formyl-6-methyl-pyridin-2-ylsulfanyl)-benzoic acid methyl |
| | ester |
| 228 | 4-(5-formyl-6-methyl-pyridin-2-yloxy)-benzoic acid methyl ester |
| 229 | [4-(5-formyl-6-methyl-pyridin-2-yloxy)-phenyl]-acetic acid |
| | methyl ester (see EXAMPLE 88) |
| 230 | 2-[4-(5-formyl-6-methyl-pyridin-2-yloxy)-phenyl)-propionic acid |
| | methyl ester |
| 231 | [4-(5-formyl-6-methyl-pyridin-2-ylsulfanyl)-phenyl]-acetic acid |
| | methyl ester |
| 232 | [3-(5-formyl-6-methyl-pyridin-2-yloxy)-phenyl]-acetic acid |
| | methyl ester |
| 233 | [4-(5-formyl-6-ethyl-pyridin-2-ylsulfanyl)-phenyl]-acetic acid |
| | methyl ester |

### EXAMPLE 223

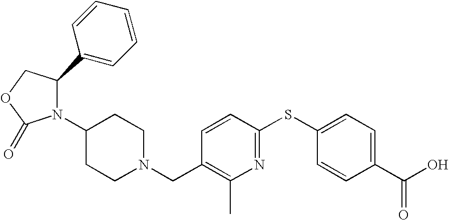

### COMPOUND 223: 4-{6-Methyl-5-[4-((R)-2-oxo-4-phenyl-oxazolidin-3-yl)-piperidin-1-yl]-pyridin-2-ylsulfanyl}-benzoic acid

COMPOUND 223 was isolated as a white solid (64 mg, 51% over 2 steps). ¹H NMR (CDCl₃) δ 1.45 (q, 1H, J=10.2 Hz), 1.57 (d, 1H, J=12.0 Hz), 1.86 (d, 1H, J=12.3 Hz), 2.21 (q, 2H, J=11.7 Hz), 2.41 (m, 1H), 2.51 (s, 3H), 3.05 (d, 1H, J=11.4 Hz), 3.31 (d, 1H, J=11.7 Hz), 3.67 (s, 2H), 3.84 (m, 1H), 4.10 (m, 1H), 4.58 (t, 1H, J=9.0 Hz), 4.78 (m, 1H), 6.40 (d, 1H, J=8.4 Hz), 7.19 (m, 3H), 7.24 (d, 2H, J=3.3 Hz), 7.41 (d, 1H, J=8.4 Hz), 7.62 (d, 2H, J=8.4 Hz), 7.98 (d, 2H, J=8.1 Hz); ES-MS m/z 504 (M+H).

### EXAMPLE 224

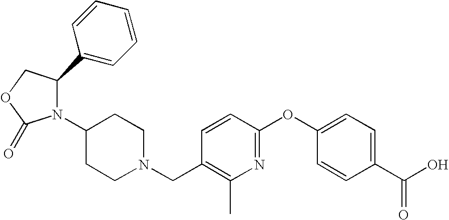

### COMPOUND 224: 4-{6-Methyl-5-[4-((R)-2-oxo-4-phenyl-oxazolidin-3-yl)-piperidin-1-ylmethyl]-pyridin-2-yloxy}-benzoic acid

COMPOUND 224 was isolated as a white solid (87 mg, 43% over 2 steps). ¹H NMR (CD₃OD) δ 1.38 (dq, 1H, J=10.2, 3.9 Hz), 1.55 (d, 1H, J=12.0 Hz), 1.70 (d, 1H, J=12.3 Hz), 2.01 (m, 3H), 2.39 (s, 3H), 2.76 (d, 1H, J=11.4 Hz), 2.90 (d, 1H, J=11.7 Hz), 3.44 (s, 2H), 3.47 (m, 1H), 4.10 (m, 1H), 4.64 (t, 1H, J=9.0 Hz), 4.87 (m, 1H), 6.68 (d, 1H, J=8.4 Hz), 7.06 (d, 2H, J=8.7 Hz), 7.39 (s, 5H), 7.63 (d, 1H, J=8.1 Hz), 8.01 (d, 2H, J=8.7 Hz); ¹³C NMR (CD₃OD) δ 20.70, 28.61, 29.62, 52.66, 52.79, 52.95, 58.11, 59.15, 71.17, 108.91, 119.44 (2C), 126.52, 127.16 (2C), 128.99, 129.29 (2C), 131.28, 131.45 (2C), 140.77, 142.71, 157.23, 157.92, 159.25, 161.92, 171.56; ES-MS m/z 488 (M+H). Anal. Calcd. for C₂₈H₂₉N₃O₅.0.4CH₂Cl₂.0.4H₃N: C, 64.56; H, 5.91; N, 9.01. Found: C, 64.22; H, 6.00; N, 8.97.

### EXAMPLE 225

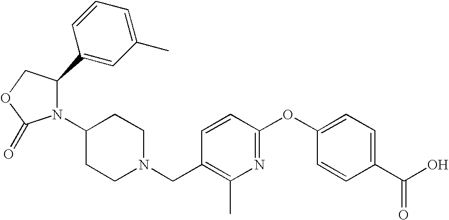

### COMPOUND 225: 4-{6-Methyl-5-[4-((R)-2-oxo-4-m-tolyl-oxazolidin-3-yl)-piperidin-1-ylmethyl]-pyridin-2-yloxy}-benzoic acid

COMPOUND 225 was isolated as a white solid (67 mg, 49% over 2 steps). ¹H NMR (CD₃OD) δ 1.95 (m, 3H), 2.36 (s, 3H), 2.45 (m, 1H), 2.45 (s, 3H), 3.00 (t, 2H, J=12.9 Hz), 3.45 (t, 2H, J=12.0 Hz), 2.58 (m, 1H), 4.13 (m, 1H), 4.24 (s, 2H), 4.66 (t, 1H, J=9.0 Hz), 4.96 (m, 1H), 6.90 (d, 1H, J=8.7 Hz), 7.20 (m, 5H), 7.33 (t, 1H, J=7.5 Hz), 7.82 (d, 1H, J=8.7 Hz), 8.07 (d, 2H, J=9.0 Hz); ES-MS m/z 502 (M+H).

### EXAMPLE 226

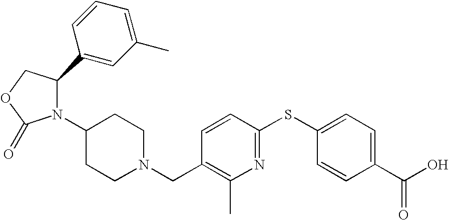

### COMPOUND 226: 4-{6-Methyl-5-[4-((R)-2-oxo-4-m-tolyl-oxazolidin-3-yl)-piperidin-1-ylmethyl]-pyridin-2-ylsulfanyl}-benzoic acid

COMPOUND 226 was isolated as a white solid (64 mg, 45% over 2 steps). ¹H NMR (CD₃OD) δ 1.57 (dq, 1H, J=12.3, 3.6 Hz), 1.74 (m, 2H), 2.14 (dq, 1H, J=9.0, 3.6 Hz), 2.35 (s, 3H), 2.38 (m, 1H), 2.48 (s, 3H), 2.98 (d, 1H, J=12.3 Hz), 3.07 (d, 1H, J=11.4 Hz), 3.50 (m, 1H), 3.69 (s, 2H), 4.09 (m, 1H), 4.63 (t, 1H, J=9.0 Hz), 4.94 (m, 1H), 6.91 (d, 1H, J=8.1 Hz), 7.18 (m, 3H), 7.29 (t, 1H, J=7.5 Hz), 7.54 (t, 1H, J=8.1 Hz), 7.55 (d, 1H, J=8.7 Hz), 8.02 (d, 2H, J=8.4 Hz); ES-MS m/z 518 (M+H).

### EXAMPLE 227

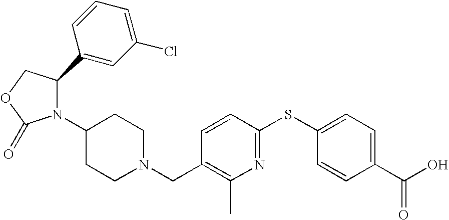

### COMPOUND 227: 4-(5-{4-[(R)-4-(3-Chloro-phenyl)-2-oxo-oxazolidin-3-yl]-piperidin-1-ylmethyl}-6-methyl-pyridin-2-ylsulfanyl)-benzoic acid

COMPOUND 227 was isolated as a white solid (57 mg, 46% over 2 steps). ¹H NMR (CD₃OD) δ 1.32 (dq, 2H, J=12.0, 3.6 Hz), 1.53 (d, 1H, J=12.3 Hz), 1.69 (d, 1H, J=11.4 Hz), 1.98 (m, 3H), 2.45 (s, 3H), 2.72 (d, 1H, J=10.5 Hz), 2.84 (d, 1H, J=8.1 Hz), 3.37 (s, 2H), 3.47 (m, 1H), 4.08 (m, 1H), 4.63 (t, 1H, J=9.0 Hz), 4.98 (m, 1H), 6.74 (d, 1H, J=7.8 Hz), 7.32 (m, 1H), 7.35-7.45 (m, 4H), 7.49 (d, 2H, J=8.1 Hz), 7.97 (d, 2H, J=8.1 Hz); ES-MS m/z 538 (M+H).

### EXAMPLE 228

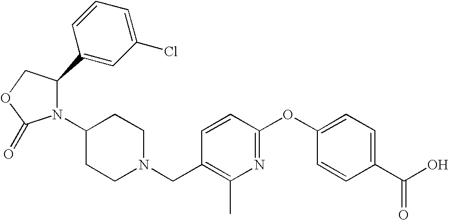

### COMPOUND 228: 4-(5-{4-[(R)-4-(3-Chloro-phenyl)-2-oxo-oxazolidin-3-yl]-piperidin-1-ylmethyl}-6-methyl-pyridin-2-yloxy)-benzoic acid

COMPOUND 228 was isolated as a white solid (27 mg, 22% over 2 steps). ¹H NMR (CD₃OD) δ 1.45 (dq, 1H, J=12.0, 3.6 Hz), 1.63 (d, 1H, J=10.8 Hz), 1.74 (d, 1H, J=12.0 Hz), 2.05 (dq, 1H, J=12.0, 3.6 Hz), 2.18 (q, 2H, J=12.0 Hz), 2.41 (s, 3H), 2.87 (d, 1H, J=12.3 Hz), 2.98 (d, 1H, J=10.8 Hz), 3.49 (m, 1H), 3.55 (s, 2H), 4.10 (m, 1H), 4.64 (t, 1H, J=9.0 Hz), 4.99 (m, 1H), 6.75 (d, 1H, J=8.1 Hz), 7.11 (d, 2H, J=8.7 Hz), 7.35 (tt, 1H, J=6.6, 2.0 Hz), 7.40 (m, 3H), 7.68 (d, 1H, J=8.1 Hz), 8.03 (d, 2H, J=8.7 Hz); ES-MS m/z 522 (M+H).

### EXAMPLE 229

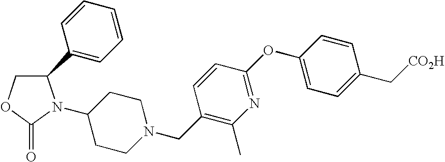

### COMPOUND 229: (4-{6-Methyl-5-[4-((R)-2-oxo-4-phenyl-oxazolidin-3-yl)-piperidin-1-ylmethyl]-pyridin-2-yloxy}-phenyl)-acetic acid

COMPOUND 229 was isolated as a colourless foam. ¹H NMR (CD₃OD) δ 1.54-1.83 (m, 3H), 2.19 (ddd, 1H, J=24.9, 12.6, 3.6 Hz), 2.35-2.47 (m, 5H), 3.00-3.04 (m, 1H), 3.11-3.15 (m, 1H), 3.49-3.60 (m, 1H), 3.63 (s, 3H), 3.73 (s, 3H), 4.15 (dd, 1H, J=8.7, 6.0 Hz), 4.69 (t, 1H, J=8.7 Hz), 5.02 (dd, 1H, J=9.0, 6.0 Hz), 6.66 (d, 1H, J=8.4 Hz), 7.06 (d, 2H, J=8.4 Hz), 7.36 (d, 2H, J=8.7 Hz), 7.42-7.49 (m, 7H), 7.66 (d, 1H, J=8.4 Hz); ES-MS m/z 502 (M+1). Anal. Calcd. for C₂₉H₃₁N₃O₅.0.4CH₂Cl₂.0.2H₂O: C, 65.50; H, 6.02; N, 7.79. Found: C, 65.43; H, 6.02; N, 7.76.

### EXAMPLE 230

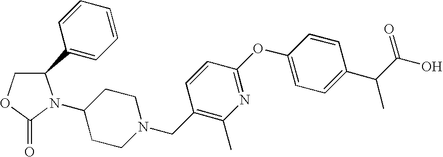

### COMPOUND 230: 2-(4-{6-Methyl-5-[4-((R)-2-oxo-4-phenyl-oxazolidin-3-yl)-piperidin-1-ylmethyl]-pyridin-2-yloxy}-phenyl)-propionic acid

A solution of (4-hydroxyphenyl)-2-propionic acid (1.43 g, 8.61 mmol) and H₂SO₄ (0.05 mL, 0.94 mmol) in MeOH (25 mL) was heated to reflux for 1.75 hours. Standard aqueous work-up afforded 2-(4-hydroxy-phenyl)-propionic acid methyl ester as a yellow oil (1.50 g, 97%).

A solution of the above phenol (606 mg, 3.36 mmol), 6-chloro-2-methyl-pyridine-3-carbaldehyde (523 mg, 3.36 mmol) and K₂CO₃ (325 mg, 2.35 mmol) in DMF (6.7 mL) was heated to 130° C. for 1 hour. Standard work-up and purification afforded 2-[4-(5-formyl-6-methyl-pyridin-2-yloxy)-phenyl]-propionic acid methyl ester (724 mg).

COMPOUND 230 was isolated as a colourless foam (61 mg, 35% over 2 steps). ¹H NMR (CD₃OD) δ 1.44-1.56 (m, 4H), 1.63-1.66 (m, 1H), 1.74-1.78 (m, 1H), 2.04-2.25 (m, 3H), 2.43 (s, 3H), 2.86-2.89 (m, 1H), 2.98-3.02 (m, 1H), 3.46-3.61 (m, 3H), 3.75 (q, 1H, J=7.2 Hz), 4.14 (dd, 1H, J=8.7, 5.7 Hz), 4.68 (t, 1H, J=9.0 Hz), 5.02 (dd, 1H, J=9.0, 5.7 Hz), 6.62 (d, 1H, J=8.1 Hz), 7.06 (d, 2H, J=8.4 Hz), 7.38-7.48 (m, 7H), 7.62 (d, 1H, J=8.4 Hz); ES-MS m/z 516 (M+1). Anal. Calcd. for C₃₀H₃₃N₃O₅.0.2CH₂Cl₂: C, 68.11; H, 6.32; N, 7.89. Found: C, 67.87; H, 6.26; N, 7.70.

### EXAMPLE 231

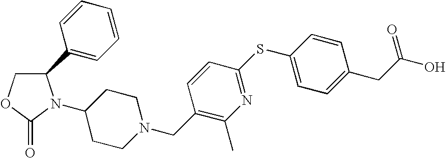

### COMPOUND 231: (4-{6-Methyl-5-[4-((R)-2-oxo-4-phenyl-oxazolidin-3-yl)-piperidin-1-ylmethyl]-pyridin-2-ylsulfanyl}-phenyl)-acetic acid

A solution of 4-methylmercaptophenylacetate (575 mg, 3.16 mmol), 6-chloro-2-methyl-pyridine-3-carbaldehyde (491 mg, 3.16 mmol) and K₂CO₃ (436 mg, 3.15 mmol) in DMF (6.3 mL) was stirred at room temperature for 2 hours. Standard work-up afforded [4-(5-formyl-6-methyl-pyridin-2-ylsulfanyl)-phenyl]-acetic acid methyl ester (941 mg, 99%).

COMPOUND 231 was isolated as a colourless foam (186 mg, 51% over 2 steps). ¹H NMR (CD₃OD) δ 1.52 (ddd, 1H, J=24.6, 12.3, 3.9 Hz), 1.64-1.68 (m, 1H), 1.74-1.78 (m, 1H), 2.02-2.32 (m, 3H), 2.49 (s, 3H), 2.88-2.92 (m, 1H), 2.99-3.03 (m, 1H), 3.47-3.57 (m, 1H), 3.59 (s, 2H), 3.67 (s, 2H), 4.14 (dd, 1H, J=8.4, 5.7 Hz), 4.68 (t, 1H, J=8.7 Hz), 5.01 (dd, 1H, J=9.0, 6.0 Hz), 6.67 (d, 1H, J=8.4 Hz), 7.38-7.54 (m, 10H). Anal. Calcd. for C₂₉H₃₁N₃SO₄.0.7H₂O: C, 65.69; H, 6.16; N, 7.92. Found: C, 65.73; H, 6.08; N, 7.88.

### EXAMPLE 232

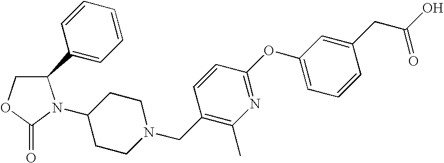

### COMPOUND 232: (3-{6-Methyl-5-[4-((R)-2-oxo-4-phenyl-oxazolidin-3-yl)-piperidin-1-ylmethyl]-pyridin-2-yloxy}-phenyl)-acetic acid

A solution of (3-hydroxy-phenyl)-acetic acid (1.26 g, 8.28 mmol) and H₂SO₄ (0.04 mL, 0.8 mmol) in MeOH (25 mL) were heated to reflux for 1.5 hours. Aqueous work-up afforded (3-hydroxy-phenyl)-acetic acid methyl ester as a colourless oil (1.33 g, 96%).

A mixture of 6-chloro-2-methyl-pyridine-3-carbaldehyde (3.0 g, 25 mmol), the above phenol (350 mg, 2.11 mmol) and K₂CO₃ (204 mg, 1.48 mmol) in DMF (4.2 mL) was heated at 130° C. for 1 hour. Aqueous work-up and purification afforded [3-(5-formyl-6-methyl-pyridin-2-yloxy)-phenyl]-acetic acid methyl ester as a yellow oil (277 mg, 46%).

COMPOUND 232 was isolated as a colourless foam (169 mg, 34% over 2 steps). ¹H NMR (CD₃OD) δ 1.46-1.66 (m, 2H), 1.73-1.77 (m, 2H), 2.13 (ddd, 1H, J=24.6, 12.3, 3.6 Hz), 2.69-2.46 (m, 5H), 2.92-2.96 (m, 1H), 3.04-3.08 (m, 1H), 3.55 (s, 3H), 3.65 (s, 2H), 4.09 (dd, 1H, J=8.7, 6.0 Hz), 4.63 (t, 1H, J=8.7 Hz), 4.96 (dd, 1H, J=9.0, 5.7 Hz), 6.56 (d, 1H, J=8.4 Hz), 6.92 (dd, 1H, J=8.1, 1.8 Hz), 7.03 (br s, 1H), 7.13 (d, 1H, J=7.5 Hz), 7.28-7.43 (m, 6H), 7.59 (d, 1H, J=8.4 Hz); ¹³C NMR (CD₃OD) δ 20.28, 27.60, 28.50, 41.80, 51.86, 52.06, 57.15, 58.82, 70.74, 107.77, 118.41, 121.16, 123.67, 125.47, 126.76, 128.62, 128.90, 129.37, 138.00, 140.17, 142.57, 154.37, 156.95, 158.75, 162.53; ES-MS m/z 502 (M+1). Anal. Calcd. for C₂₉H₃₁N₃O₅.0.1H₂O.0.1CH₂Cl₂: C, 68.28; H, 6.18; N, 8.21. Found: C, 68.32; H, 6.18; N, 8.18.

### EXAMPLE 233

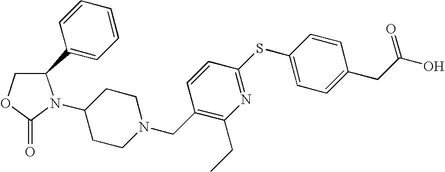

### COMPOUND 233: (4-{6-Ethyl-5-[4-((R)-2-oxo-4-phenyl-oxazolidin-3-yl)-piperidin-1-yl]-pyridin-2-ylsulfanyl}-phenyl)-acetic acid

COMPOUND 233 was isolated as a colourless foam (69 mg, 36% over 2 steps). ¹H NMR (CD₃OD) δ 1.14 (t, 3H, J=7.5 Hz), 1.40-1.52 (m, 1H), 1.61-1.65 (m, 1H), 1.71-1.75 (m, 1H), 1.99-2.30 (m, 3H), 2.74 (q, 2H, J=7.5 Hz), 2.84-2.88 (m, 1H), 2.97-3.00 (m, 1H), 3.44-3.52 (m, 1H), 3.58 (s, 2H), 3.65 (s, 2H), 4.11 (dd, 1H, J=8.7, 6.0 Hz), 4.64 (t, 1H, J=8.7 Hz), 4.97 (dd, 1H, J=8.7, 5.7 Hz), 6.66 (d, 1H, J=8.1 Hz), 7.37-7.44 (m, 8H), 7.50 (d, 2H, J=7.8 Hz); ¹³C NMR (CD₃OD) δ 14.00, 28.32, 28.65, 29.52, 42.38, 52.82, 53.01, 53.17, 58.11, 60.02, 71.87, 119.30, 125.15, 127.96, 129.65, 129.80, 130.08, 131.76, 135.97, 138.43, 140.87, 141.22, 159.83, 162.11, 163.90, 175.68; ES-MS m/z 532 (M+1).

Examples 234 to 239 were prepared following the scheme illustrated below. RCHO is as defined in the table and Y is as defined in the individual examples.

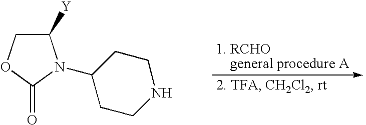

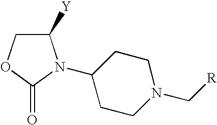

| Example | RCHO |
|---|---|
| 234 | [4-(5-formyl-6-methyl-pyridin-2-yloxy)-phenoxy]-acetic acid |
| | tert-butyl ester |
| 235 | [4-(5-formyl-6-methyl-pyridin-2-yloxy)-phenylsulfanyl]-acetic |
| | acid tert-butyl ester |
| 236 | [4-(5-formyl-pyridin-2-ylsulfanyl)-phenoxy]-acetic acid |
| | tert-butyl ester |
| 237 | [4-(5-formyl-6-methyl-pyridin-2-ylsulfanyl)-phenoxy]-acetic |
| | acid tert-butyl ester |
| 238 | 4-(6-ethyl-5-formyl-pyridin-2-yloxy)-benzoic acid tert-butyl |
| | ester |
| 239 | 4-(5-formyl-6-methyl-pyridin-2-yloxy)-benzoic acid tert-butyl |
| | ester |

### EXAMPLE 234

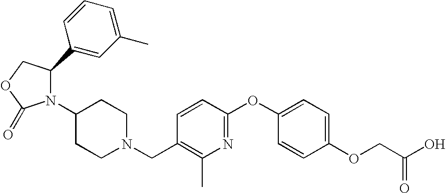

### COMPOUND 234: (4-{6-Methyl-5-[4-((R)-2-oxo-4-m-tolyl-oxazolidin-3-yl)-piperidin-1-ylmethyl]-pyridin-2-yloxy}-phenoxy)-acetic acid

COMPOUND 234 was isolated as a white solid (49 mg, 57%). ¹H NMR (CD₃OD) δ 1.97 (m, 3H), 2.40 (s, 3H), 2.44 (m, 1H), 2.48 (s, 3H), 3.08 (dt, 2H, J=12.0, 2.7 Hz), 3.49 (t, 2H, J=11.4 Hz), 3.60 (m, 1H), 4.16 (m, 1H), 4.27 (s, 2H), 4.70 (m, 1H), 4.70 (s, 2H), 4.98 (m, 1H), 6.74 (d, 1H, J=8.7 Hz), 7.04 (m, 4H), 7.26 (m, 3H), 7.36 (t, 1H, J=7.5 Hz), 7.76 (d, 1H, J=8.7 Hz); ES-MS m/z 532 (M+H).

### EXAMPLE 235

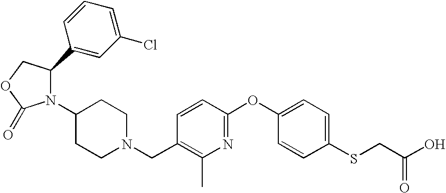

### COMPOUND 235: [4-(5-{4-[(R)-4-(3-Chloro-phenyl)-2-oxo-oxazolidin-3-yl]-piperidin-1-ylmethyl}-6-methyl-pyridin-2-yloxy)-phenylsulfanyl]-acetic acid

COMPOUND 235 was isolated as a white solid (25 mg, 30% over 2 steps). ¹H NMR (CD₃OD) δ 1.82 (dq, 1H, J=12.0, 3.6 Hz), 1.92 (d, 2H, J=9.6 Hz), 2.33 (dq, 1H, J=12.0, 3.6 Hz), 2.47 (s, 3H), 2.85 (m, 2H), 3.38 (m, 2H), 3.61 (tt, 1H, J=12.0, 3.6 Hz), 3.71 (s, 2H), 4.11 (s, 2H), 4.16 (m, 1H), 4.71 (t, 1H, J=9.0 Hz), 5.03 (m, 1H), 6.78 (d, 1H, J=8.4 Hz), 7.09 (d, 2H, J=8.4 Hz), 7.41 (tt, 1H, J=12.0, 3.6 Hz), 7.49 (t, 5H, J=8.7 Hz), 7.77 (d, 1H, J=8.4 Hz); ES-MS m/z 568 (M+H).

### EXAMPLE 236

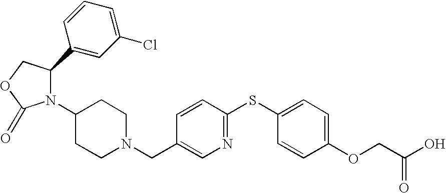

### COMPOUND 236: [4-(5-{4-[(R)-4-(3-Chloro-phenyl)-2-oxo-oxazolidin-3-yl]-piperidin-1-ylmethyl}-pyridin-2-ylsulfanyl)-phenoxy]-acetic acid

COMPOUND 236 was isolated as a white solid (34 mg, 43% over 2 steps). ¹H NMR (CD₃OD) δ 1.93 (m, 3H), 2.36 (dq, 1H, J=12.0, 3.6 Hz), 2.94 (dt, 2H, J=12.6, 2.7 Hz), 3.40 (q, 2H, J=12.0 Hz), 3.58 (m, 1H), 4.17 (m, 1H), 4.17 (s, 2H), 4.71 (t, 1H, J=9.0 Hz), 4.73 (s, 2H), 5.02 (m, 1H), 6.91 (d, 1H, J=8.1 Hz), 7.09 (d, 2H, J=8.7 Hz), 7.41 (m, 1H), 7.47 (m, 1H), 7.47 (d, 2H, J=7.5 Hz), 7.56 (d, 2H, J=9.9 Hz), 7.63 (dd, 1H, J=8.4, 2.1 Hz), 8.40 (s, 1H); ES-MS m/z 554 (M+H).

### EXAMPLE 237

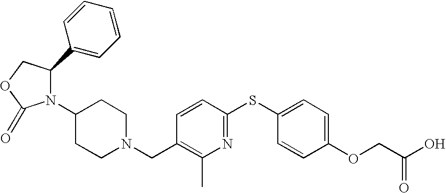

### COMPOUND 237: (4-{6-Methyl-5-[4-((R)-2-oxo-4-phenyl-oxazolidin-3-yl)-piperidin-1-ylmethyl]-pyridin-2-ylsulfanyl}-phenoxy)-acetic acid

COMPOUND 237 was isolated as a white solid (44 mg, 48% over 2 steps). ¹H NMR (CD₃OD) δ 1.83 (m, 1H), 1.90 (d, 2H, J=11.7 Hz), 2.34 (dq, 1H, J=12.0, 3.6 Hz), 2.54 (s, 3H), 2.86 (t, 2H, J=12.3 Hz), 3.58 (m, 1H), 4.07 (s, 2H), 4.14 (m, 1H), 4.65 (s, 2H), 4.70 (t, 1H, J=9.0 Hz), 5.00 (m, 1H), 6.63 (d, 1H, J=8.1 Hz), 7.07 (d, 2H, J=8.7 Hz), 7.45 (s, 5H), 7.46-7.55 (m, 3H); ES-MS m/z 534 (M+H).

### EXAMPLE 238

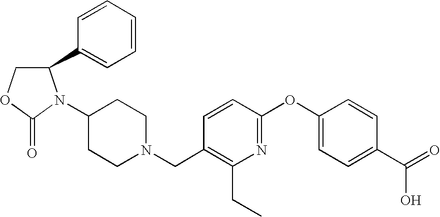

### COMPOUND 238: 4-{6-Ethyl-5-[4-((R)-2-oxo-4-phenyl-oxazolidin-3-yl)-piperidin-1-ylmethyl]-pyridin-2-yloxy}-benzoic acid

COMPOUND 238 was isolated as a yellow foam (48.5 mg, 34% over 2 steps). ¹H NMR (CD₃OD) δ 1.11 (t, 3H, J=7.5 Hz), 1.55-1.67 (m, 1H), 1.72-1.83 (m, 2H), 2.13-2.26 (m, 1H), 2.45-2.56 (m, 2H), 2.71 (q, 2H, J=7.5 Hz), 3.06 (d, 1H, J=11.1 Hz), 3.17 (d, 1H, J=11.4 Hz), 3.50-3.58 (m, 1H), 3.82 (s, 2H), 4.12 (dd, 1H, J=8.4, 6.0 Hz), 4.66 (t, 1H, J=8.9 Hz), 4.98 (dd, 1H, J=8.7, 6.0 Hz), 6.79 (d, 1H, J=8.1 Hz), 7.15 (d, 2H, J=5.7 Hz), 7.40 (m, 5H), 7.73 (d, 1H, J=8.4 Hz), 8.04 (d, 2H, J=4.5 Hz); ¹³C NMR (CD₃OD) δ 11.77, 26.40, 26.51, 27.27, 50.57, 51.18, 51.29, 55.85, 58.45, 70.19, 108.31, 119.18, 121.08, 126.25, 128.14, 128.40, 130.63, 139.30, 142.62, 157.30, 158.14, 161.36, 161.73; ES-MS m/z 502 (M+H); Anal. Calcd. for C₂₉H₃₁N₃O₅.2.5CH₂Cl₂: C, 53.00; H, 5.08; N, 5.89. Found: C, 52.77; H, 5.14; N, 6.01.

### EXAMPLE 239

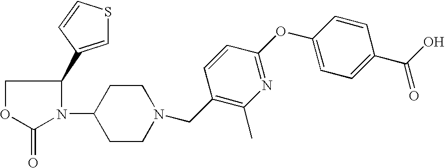

### COMPOUND 239: 4-{6-Methyl-5-[4-((R)-2-oxo-4-thiophen-3-yl-oxazolidin-3-yl)-piperidin-1-ylmethyl]-pyridin-2-yloxy}-benzoic acid

Using general procedure C, ((R)-2-hydroxy-1-thiophen-3-yl-ethyl)-carbamic acid tert-butyl ester (600 mg, 2.47 mmol) and TFA (2 mL) in CH₂Cl₂ (4 mL) gave crude (R)-2-amino-2-thiophen-3-yl-ethanol as a colorless oil (295 mg, 84%).

Using general procedure A, the above amine (410 mg, 2.06 mmol), sodium triacetoxyborohydride (212 mg, 2.88 mmol) and acetic acid (59 μL, 1.0 mmol) in CH₂Cl₂ (10 mL) gave crude 4-((R)-2-hydroxy-1-thiophen-3-yl-ethylamino)-piperidine-1-carboxylic acid tert-butyl ester as a white foam (793 mg, quant).

Following general procedure K: to the solution of the above substrate (793 mg, 2.43 mmol) in CH₂Cl₂ (10 mL) and triethylamine (677 μL, 4.86 mmol) at 0° C. under stirring was added triphosgene (360 mg, 1.22 mmol) in CH₂Cl₂ (6 mL) portion-wise. The mixture was stirred for 1.5 h to afford crude 4-((R)-2-oxo-4-thiophen-3-yl-oxazolidin-3-yl)-piperidine-1-carboxylic acid tert-butyl ester as a yellow oil (673 mg, 79%).

Using general procedure C, 4-((R)-2-oxo-4-thiophen-3-yl-oxazolidin-3-yl)-piperidine-1-carboxylic acid tert-butyl ester (673 mg, 1.91 mmol) and TFA (2 mL) in CH₂Cl₂ (4 mL) gave crude (R)-3-piperidin-4-yl-4-thiophen-3-yl-oxazolidin-2-one as a white foam (340 mg, 71%).

COMPOUND 239 was isolated as a white foam (57 mg, 23% over 2 steps). ¹H NMR (CD₃OD) δ 1.29 (s, 1H), 1.79-1.83 (m, 3H), 2.28-2.33 (m, 1H), 2.45 (s, 3H), 2.73-2.81 (m, 2H), 3.54-3.55 (m, 1H), 4.02 (s, 2H), 4.17 (dd, 1H, J=8.7, 6 Hz), 4.61 (t, 1H, J=8.7 Hz), 5.14 (dd, 1H, J=8.7, 6 Hz), 5.50 (s, 1H), 6.87 (d, 1H, J=8.4 Hz), 7.15-7.18 (m, 3H), 7.51-7.55 (m, 2H), 7.79 (d, 1H, J=8.4 Hz), 8.07 (d, 2H, J=8.7 Hz).

### EXAMPLE 240

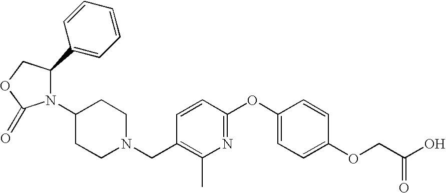

### COMPOUND 240: (4-{6-Methyl-5-[4-((R)-2-oxo-4-phenyl-oxazolidin-3-yl)-piperidin-1-ylmethyl]-pyridin-2-yloxy}-phenoxy)-acetic acid

To a solution of (R)-3-{1-[6-(4-hydroxy-phenoxy)-2-methyl-pyridin-3-ylmethyl]-piperidin-4-yl}-4-phenyl-oxazolidin-2-one (COMPOUND 250) (59.1 mg, 0.129 mmol) in dry THF (1.0 mL) was added NaH (60% dispersion in mineral oil, 7.7 mg, 0.19 mmol). After stirring for 5 min, t-butyl bromoacetate (22.9 μL, 0.155 mmol) was added to the mixture. Standard work-up and purification provided (4-{6-methyl-5-[4-((R)-2-oxo-4-phenyl-oxazolidin-3-yl)-piperidin-1-ylmethyl]-pyridin-2-yloxy}-phenoxy)-acetic acid tert-butyl ester (55.3 mg, 75%).

The above product was treated with TFA (0.4 mL) in dichloromethane (0.6 mL) at rt for 2 h to provide COMPOUND 240 as a white powder (45.6 mg, 91%). ¹H NMR (CD₃OD) δ 1.78-1.89 (m, 3H), 2.29-2.40 (m, 1H), 2.41 (s, 3H), 2.82-2.92 (m, 2H), 3.26-3.32 (m, 1H), 3.38 (d, 1H, J=11.7 Hz), 3.61 (m, 1H), 4.07-4.13 (m, 3H), 4.45 (br s, 2H), 4.65 (t, 1H, J=9.0 Hz), 4.98 (dd, 1H, J=8.7, 6.0 Hz), 6.59 (d, 1H, J=8.4 Hz), 6.90 (d, 2H, J=8.4 Hz), 6.97 (d, 2H, J=8.4 Hz), 7.33-7.44 (m, 4H), 7.70 (d, 1H, J=8.4 Hz); ¹³C NMR (CD₃OD) δ 22.51, 28.12, 28.71, 52.06, 53.17, 53.32, 57.96, 60.92, 72.62, 109.45, 117.24, 120.82, 123.62, 128.67, 130.61, 130.88, 141.55, 145.41, 149.27, 157.55, 159.52, 160.40, 165.76; ES-MS m/z 518 (M+H). Anal. Calcd. for C₂₉H₃₁N₃O₆.1.0CH₂Cl₂: C, 59.80; H, 5.52; N, 6.97. Found: C, 59.74; H, 5.60; N, 6.81.

### EXAMPLE 241

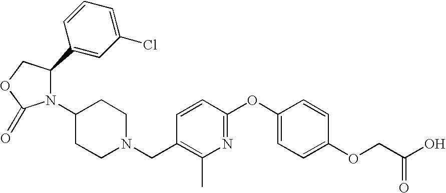

### COMPOUND 241: [4-(5-{4-[(R)-4-(3-Chloro-phenyl)-2-oxo-oxazolidin-3-yl]-piperidin-1-ylmethyl}-6-methyl-pyridin-2-yloxy)-phenoxy]-acetic acid

To a solution of (R)-4-(3-chloro-phenyl)-3-{1-[6-(4-hydroxy-phenoxy)-2-methyl-pyridin-3-ylmethyl]-piperidin-4-yl}-oxazolidin-2-one (COMPOUND 258) (66.8 mg, 0.135 mmol) in dry THF (1.0 mL) was added NaH (60%, 8.1 mg, 0.203 mmol). The solution was stirred at rt for 10 min then t-butyl-bromoacetate (23.9 μL, 0.162 mmol) was added. The mixture was stirred at rt for 2 h. Standard work-up and purification afforded [4-(5-{4-[(R)-4-(3-chloro-phenyl)-2-oxo-oxazolidin-3-yl]-piperidin-1-ylmethyl}-6-methyl-pyridin-2-yloxy)-phenoxy]-acetic acid tert-butyl ester (53.3 mg, 65%).

The above product (53.3 mg, 0.088 mmol) was treated with TFA (0.3 mL) in CH₂Cl₂ (0.5 mL) at rt for 2 h to give COMPOUND 241 as a yellow powder (50.0 mg, 100%). ¹H NMR (CD₃OD) δ 1.85-2.03 (m, 3H), 2.37-2.55 (m, 1H), 2.48 (s, 3H), 3.10-3.18 (m, 2H), 3.53 (m, 2H), 3.71 (m, 1H), 4.14 (dd, 1H, J=8.7, 6.0 Hz), 4.31 (s, 2H), 4.70 (s, 2H), 4.71 (t, 1H, J=8.7 Hz), 5.04 (dd, 1H, J=9.0, 6.0 Hz), 6.72 (d, 1H, J=8.4 Hz), 7.01 (d, 2H, J=9.0 Hz), 7.07 (d, 2H, J=9.0 Hz), 7.37-7.48 (m, 4H), 7.80 (d, 1H, J=8.4 Hz); ¹³C NMR (CD₃OD) δ 22.6, 27.8, 28.3, 51.6, 53.1, 53.2, 57.8, 60.4, 66.8, 72.4, 109.8, 117.3, 119.5, 123.8, 127.0, 128.8, 130.8, 132.6, 136.6, 143.9, 145.7, 149.5, 157.2, 159.8, 160.2, 166.0, 173.1; ES-MS m/z 552 (M+H). Anal. Calcd. for C₂₉H₃₀N₃O₆Cl.1.6CH₂Cl₂: C, 53.43; H, 4.86; N, 6.11. Found: C, 53.63; H, 4.63; N, 5.76.

### EXAMPLE 242

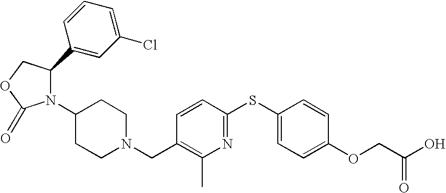

### COMPOUND 242: [4-(5-{4-[(R)-4-(3-Chloro-phenyl)-2-oxo-oxazolidin-3-yl]-piperidin-1-ylmethyl}-6-methyl-pyridin-2-ylsulfanyl)-phenoxy]-acetic acid

To a solution of (R)-4-(3-chloro-phenyl)-3-{1-[6-(4-hydroxy-phenylsulfanyl)-2-methyl-pyridin-3-ylmethyl]-piperidin-4-yl}-oxazolidin-2-one (COMPOUND 259) (70.1 mg, 0.138 mmol) in dry THF (1.0 mL) was added NaH (60%, 8.3 mg, 0.206 mmol). The solution was stirred at rt for 10 min then t-butyl-bromoacetate (24.5 μL, 0.166 mmol) was added. The mixture was stirred at rt for 1.5 h. Standard work-up and purification gave [4-(5-{4-[(R)-4-(3-chloro-phenyl)-2-oxo-oxazolidin-3-yl]-piperidin-1-ylmethyl}-6-methyl-pyridin-2-ylsulfanyl)-phenoxy]-acetic acid tert-butyl ester (61.6 mg, 72%).

The above product (61.6 mg, 0.099 mmol) was treated with TFA (0.3 mL) in CH₂Cl₂ (0.5 mL) at rt for 2 h to give COMPOUND 242 as a white powder (36.3 mg, 65%). ¹H NMR (CD₃OD) δ 1.85-2.03 (m, 3H), 2.37-2.49 (m, 1H), 2.55 (s, 3H), 3.08 (m, 2H), 3.46 (m, 2H), 3.71 (m, 1H), 4.13 (dd, 1H, J=8.7, 6.0 Hz), 4.23 (s, 2H), 4.68 (s, 2H), 4.69 (t, 1H, J=8.7 Hz), 5.02 (dd, 1H, J=9.0, 6.0 Hz), 6.62 (d, 1H, J=8.4 Hz), 7.05 (d, 2H, J=8.4 Hz), 7.36-7.57 (m, 8H); ¹³C NMR (CD₃OD) δ 21.2, 26.4, 27.0, 50.3, 51.7, 51.9, 56.5, 58.9, 65.6, 70.9, 116.4, 118.1, 119.9, 120.9, 125.6, 127.4, 129.3, 131.1, 135.2, 137.5, 141.1, 142.5, 158.8, 159.9, 160.1, 164.7, 172.1; ES-MS m/z 568 (M+H). Anal. Calcd. for C₂₉H₃₀N₃O₅SCl.0.9CH₂Cl₂: C, 55.72; H, 4.97; N, 6.52. Found: C, 55.87; H, 4.94; N, 6.53.

### EXAMPLE 243

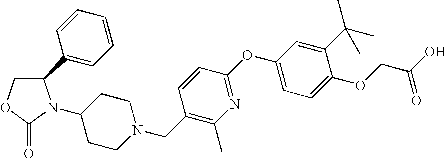

### COMPOUND 243: (2-tert-Butyl-4-{6-methyl-5-[4-((R)-2-oxo-4-phenyl-oxazolidin-3-yl)-piperidin-1-ylmethyl]-pyridin-2-yloxy}-phenoxy)-acetic acid

To a solution of (R)-3-{1-[6-(3-tert-butyl-4-hydroxy-phenoxy)-2-methyl-pyridin-3-ylmethyl]-piperidin-4-yl}-4-phenyl-oxazolidin-2-one (COMPOUND 222) (120 mg, 0.233 mmol) in THF (2.3 mL) was added NaH (60%, 14 mg, 0.35 mmol) and the mixture was stirred at room temperature for 20 minutes. A solution of tert-butyl bromoacetate (55 mg, 0.33 mmol) in THF (0.5 mL) was added and the mixture was heated at 50° C. for 1.2 hours. Standard work-up and purification afforded (2-tert-butyl-4-{6-methyl-5-[4-((R)-2-oxo-4-phenyl-oxazolidin-3-yl)-piperidin-1-ylmethyl]-pyridin-2-yloxy}-phenoxy)-acetic acid tert-butyl ester. Following general procedure C, the tert-butyl ester was treated with TFA (1 mL) in CH₂Cl₂ (1 mL) and stirred at room temperature for 2 hours. Standard work-up and purification afforded COMPOUND 243 as a white solid (98 mg, 73% over 2 steps). ¹H NMR (CDCl₃) δ 1.25-1.40 (m, 11H), 1.55-1.57 (m, 1H), 1.88-1.93 (m, 1H), 2.38-2.68 (m, 5H), 3.29-3.32 (m, 1H), 3.48-3.51 (m, 1H), 3.94-3.98 (m, 3H), 4.11-4.16 (m, 1H), 4.52-4.63 (m, 3H), 4.78-4.83 (m, 1H), 6.45 (m, 1H, J=8.1 Hz), 6.64-6.67 (m, 1H), 6.76-6.78 (m, 1H), 7.03-7.04 (m, 1H), 7.29-7.38 (m, 5H), 7.67 (d, 1H, J=8.1 Hz); ¹³C NMR (CDCl₃) δ 22.57, 27.09, 28.44, 30.09, 35.41, 51.24, 51.82, 52.06, 56.86, 58.39, 67.11, 71.17, 107.64, 113.43, 119.29, 119.84, 120.63, 127.14, 129.57, 129.76, 140.34, 140.65, 143.31, 147.32, 154.89, 157.42, 158.47, 164.29, 173.27; ES-MS m/z 574 (M+1). Anal. Calcd. for C₃₃H₃₉N₃O₆.0.79CH₂Cl₂: C, 63.33; H, 6.38; N, 6.56. Found: C, 63.33; H, 6.40; N, 6.57.

Examples 244 to 249 were prepared following the scheme illustrated below. RNH₂ is as defined in the table and Y and Z are as defined in the individual examples.

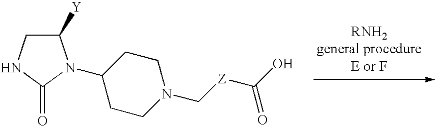

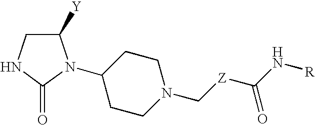

| Example | RNH₂ |
|---|---|
| 244 | Cyclopropylamine |
| 245 | Cyclopropylamine |
| 246 | methylamine hydrochloride |
| 247 | methoxylamine hydrochloride |
| 248 | methylamine hydrochloride |
| 249 | methylamine hydrochloride |

### EXAMPLE 244

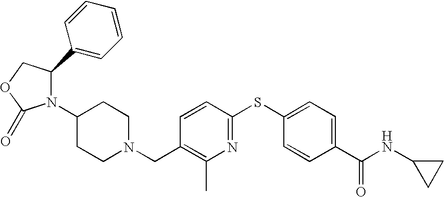

### COMPOUND 244: N-Cyclopropyl-4-{6-methyl-5-[4-((R)-2-oxo-4-phenyl-oxazolidin-3-yl)-piperidin-1-ylmethyl]-pyridin-2-ylsulfanyl}-benzamide

Following general procedure F: 4-{6-methyl-5-[4-((R)-2-oxo-4-phenyl-oxazolidin-3-yl)-piperidin-1-ylmethyl]-pyridin-2-ylsulfanyl}-benzoic acid (COMPOUND 223) afforded COMPOUND 244 as white solid (26 mg, 52%). ¹H NMR (CDCl₃) δ 0.63 (m, 2H), 0.88 (q, 2H, J=6.6 Hz), 1.21 (dq, 1H, J=12.3, 3.9 Hz), 1.47 (d, 1H, J=12.0 Hz), 1.70 (d, 1H, J=12.3 Hz), 1.89 (q, 2H, J=11.7 Hz), 1.96 (m, 1H), 2.44 (s, 3H), 2.63 (d, 1H, J=11.4 Hz), 2.80 (d, 1H, J=11.7 Hz), 2.90 (m, 1H), 3.29 (s, 2H), 3.59 (m, 1H), 4.09 (m, 1H), 4.57 (t, 1H, J=9.0 Hz), 4.78 (m, 1H), 6.23 (s, 1H), 6.74 (d, 1H, J=7.8 Hz), 7.26-7.42 (m, 6H), 7.54 (d, 2H, J=8.4 Hz), 7.72 (d, 1H, J=8.4 Hz); ES-MS m/z 543 (M+H).

### EXAMPLE 245

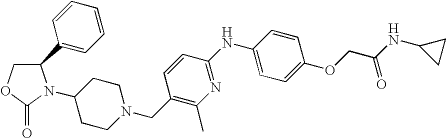

### COMPOUND 245: N-Cyclopropyl-2-(4-{6-methyl-5-[4-((R)-2-oxo-4-phenyl-oxazolidin-3-yl)-piperidin-1-ylmethyl]-pyridin-2-ylamino}-phenoxy)-acetamide

Following general procedure E: (4-{6-methyl-5-[4-((R)-2-oxo-4-phenyl-oxazolidin-3-yl)-piperidin-1-ylmethyl]-pyridin-2-ylamino}-phenoxy)-acetic acid (COMPOUND 262) afforded COMPOUND 245 as a tan foam (18 mg, 35%). ¹H NMR (CDCl₃) δ 0.55-0.61 (m, 2H), 0.81-0.87 (m, 2H), 1.14-1.28 (m, 1H), 1.44-1.49 (m, 1H), 1.68-1.72 (m, 1H), 1.77-2.04 (m, 3H), 2.37 (s, 3H), 2.65-2.69 (m, 1H), 2.75-2.86 (m, 2H), 3.20-3.30 (m, 2H), 3.55-3.64 (m, 1H), 4.08 (dd, 1H, J=8.7, 5.7 Hz), 4.45 (s, 2H), 4.57 (t, 1H, J=8.7 Hz), 4.78 (dd, 1H, J=9.0, 5.7 Hz), 6.29 (s, 1H), 6.49 (d, 1H, J=8.4 Hz), 6.64 (br s, 1H), 6.83-6.89 (m, 2H), 7.18-7.25 (m, 3H), 7.30-7.42 (m, 5H); ¹³C NMR (CDCl₃) δ 6.51, 21.88, 22.12, 29.27, 30.48, 52.69, 52.77, 53.19, 58.52, 59.21, 67.91, 70.51, 104.25, 115.43, 122.40, 122.69, 126.75, 128.90, 129.17, 135.25, 139.62, 140.53, 153.03, 154.78, 156.47, 158.07, 169.69; ES-MS m/z 578 (M+Na). Anal. Calcd. for C₃₂H₃₇N₅O₄.0.2CH₂Cl₂.0.2CH₄O: C, 67.20; H, 6.65; N, 12.09. Found: C, 67.23; H, 6.50; N, 11.82.

### EXAMPLE 246

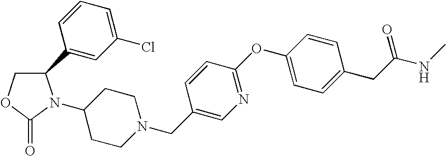

### COMPOUND 246: 2-[4-(5-{4-[(R)-4-(3-Chloro-phenyl)-2-oxo-oxazolidin-3-yl]-piperidin-1-ylmethyl}-pyridin-2-yloxy)-phenyl]-N-methyl-acetamide

A solution of 4-hydroxyphenylacetate (510 mg, 3.07 mmol), 6-bromo-pyridine-3-carbaldehyde (571 mg, 3.07 mmol) and K₂CO₃ (297 mg, 2.15 mmol) in DMF (3.0 mL) was heated to 130° C. for 1 hour. Aqueous work-up and purification afforded [4-(5-formyl-pyridin-2-yloxy)-phenyl]-acetic acid methyl ester (814 mg).

Following general procedure A, (R)-4-(3-chloro-phenyl)-3-piperidin-4-yl-oxazolidin-2-one (160 mg, 0.570 mmol) and the above aldehyde (201 mg) afforded [4-(5-{4-[(R)-4-(3-chloro-phenyl)-2-oxo-oxazolidin-3-yl]-piperidin-1-ylmethyl}-pyridin-2-yloxy)-phenyl]-acetic acid methyl ester (183 mg, 60%) following work-up and purification. Following general procedure H, the above methyl ester (183 mg, 0.341 mmol) afforded the carboxylic acid as a yellow foam (178 mg, quant).

Following general procedure E: the above acid afforded COMPOUND 246 as a colourless foam (49 mg, 80%). ¹H NMR (CDCl₃) δ 1.26 (ddd, 1H, J=24.6, 12.3, 4.2 Hz), 1.49-1.54 (m, 1H), 1.71-1.75 (m, 1H), 1.82-2.04 (m, 3H), 2.70-2.74 (m, 1H), 2.78 (d, 3H, J=4.8 Hz), 2.86-2.89 (m, 1H), 3.37 (s, 2H), 3.57-3.67 (m, 3H), 4.04 (dd, 1H, J=8.7, 5.1 Hz), 4.57 (t, 1H, J=8.7 Hz), 4.76 (dd, 1H, J=9.0, 5.4 Hz), 5.45 (br s, 1H), 6.87 (d, 1H, J=8.4 Hz), 7.09-7.12 (m, 1H), 7.20-7.34 (m, 6H), 7.61 (dd, 1H, J=8.1, 2.1 Hz), 8.00 (d, 1H, J=2.1 Hz); ¹³C NMR (CDCl₃) δ 26.52, 29.24, 30.61, 43.09, 52.57, 52.70, 53.06, 57.88, 59.13, 70.29, 111.40, 121.55, 124.74, 126.80, 128.55, 129.18, 130.62, 130.85, 130.97, 135.16, 140.53, 142.80, 147.64, 153.53, 157.92, 162.79, 171.53; ES-MS m/z 557 (M+Na). Anal. Calcd. for C₂₉H₃₁N₄ClO₄.0.3CH₂Cl₂.0.1CH₄O: C, 62.64; H, 5.72; N, 9.94. Found: C, 62.58; H, 5.74; N, 9.92.

### EXAMPLE 247

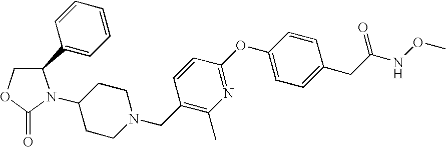

### COMPOUND 247: N-Methoxy-2-(4-{6-methyl-5-[4-((R)-2-oxo-4-phenyl-oxazolidin-3-yl)-piperidin-1-ylmethyl]-pyridin-2-yloxy}-phenyl)-acetamide

Following general procedure E: (4-{6-methyl-5-[4-((R)-2-oxo-4-phenyl-oxazolidin-3-yl)-piperidin-1-ylmethyl]-pyridin-2-yloxy}-phenyl)-acetic acid (COMPOUND 229) afforded COMPOUND 247 as a white foam (30 mg, 31%). ¹H NMR (CDCl₃) δ 1.17-1.30 (m, 1H), 1.46-1.51 (m, 1H), 1.69-1.73 (m, 1H), 1.85-2.04 (m, 3H), 2.38 (s, 3H), 2.65-2.68 (m, 1H), 2.82-2.85 (m, 1H), 3.31 (s, 2H), 3.53-3.64 (m, 3H), 3.74 (s, 3H), 4.09 (dd, 1H, J=8.7, 5.7 Hz), 4.57 (t, 1H, J=9.0 Hz), 4.79 (dd, 1H, J=8.7, 5.7 Hz), 6.55 (d, 1H, J=7.8 Hz), 7.08 (d, 2H, J=8.4 Hz), 7.25-7.46 (m, 8H), 8.23 (br s, 1H); ¹³C NMR (CDCl₃) δ 22.17, 29.61, 30.76, 40.58, 53.19, 53.30, 53.46, 58.98, 59.23, 64.81, 70.93, 108.09, 121.30, 127.15, 129.35, 129.60, 130.01, 130.90, 140.79, 141.35, 154.38, 156.92, 158.50, 162.00, 169.00; ES-MS m/z 531 (M+1). Anal. Calcd. for C₃₀H₃₄N₄O₅.0.2CH₂Cl₂: C, 66.24; H, 6.33; N, 10.23. Found: C, 66.37; H, 6.35; N, 10.15.

### EXAMPLE 248

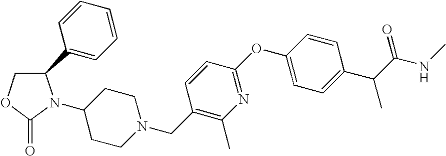

### COMPOUND 248: N-Methyl-2-(4-{6-methyl-5-[4-((R)-2-oxo-4-phenyl-oxazolidin-3-yl)-piperidin-1-ylmethyl]-pyridin-2-yloxy}-phenyl)-propionamide

Following general procedure E: 2-(4-{6-methyl-5-[4-((R)-2-oxo-4-phenyl-oxazolidin-3-yl)-piperidin-1-ylmethyl]-pyridin-2-yloxy}-phenyl)-propionic acid (COMPOUND 230) afforded COMPOUND 248 as a colourless foam (70 mg, 66%). ¹H NMR (CDCl₃) δ 1.24 (ddd, 1H, J=24.3, 12.3, 3.9 Hz), 1.47-1.54 (m, 4H), 1.69-1.73 (m, 1H), 1.85-2.04 (m, 3H), 2.39 (s, 3H), 2.65-2.68 (m, 1H), 2.76 (d, 3H, J=4.8 Hz), 2.82-2.85 (m, 1H), 3.31 (s, 2H), 3.51-3.64 (m, 2H), 4.09 (dd, 1H, J=8.4, 5.7 Hz), 4.57 (t, 1H, J=8.7 Hz), 4.79 (dd, 1H, J=8.7, 5.4 Hz), 5.38 (br s, 1H), 6.54 (d, 1H, J=8.1 Hz), 7.07 (d, 2H, J=8.7 Hz), 7.29-7.45 (m, 8H); ¹³C NMR (CDCl₃) δ 18.67, 21.83, 26.51, 29.22, 30.38, 46.43, 52.81, 52.92, 53.08, 58.60, 58.86, 70.52, 107.68, 120.76, 126.76, 126.84, 128.89, 128.94, 129.20, 137.09, 140.43, 140.91, 153.84, 156.54, 158.06, 161.60, 174.83; ES-MS m/z 529 (M+1). Anal. Calcd. for C₃₁H₃₆N₄O₄.0.3CH₂Cl₂: C, 67.84; H, 6.66; N, 10.11. Found: C, 67.96; H, 6.70; N, 10.13.

### EXAMPLE 249

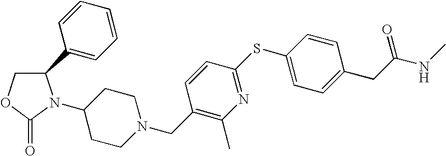

### COMPOUND 249: N-Methyl-2-(4-{6-methyl-5-[4-((R)-2-oxo-4-phenyl-oxazolidin-3-yl)-piperidin-1-ylmethyl]-pyridin-2-ylsulfanyl}-phenyl)-acetamide

Following general procedure E: (4-{6-methyl-5-[4-((R)-2-oxo-4-phenyl-oxazolidin-3-yl)-piperidin-1-ylmethyl]-pyridin-2-ylsulfanyl}-phenyl)-acetic acid (COMPOUND 231) afforded COMPOUND 249 as a colourless foam (112 mg, 87%). ¹H NMR (CDCl₃) δ 1.21 (ddd, 1H, J=24.6, 12.3, 3.9 Hz), 1.45-1.48 (m, 1H), 1.67-1.71 (m, 1H), 1.84-2.02 (m, 3H), 2.44 (s, 3H), 2.61-2.65 (m, 1H), 2.79-2.80 (m, 4H), 3.27 (s, 2H), 3.53-3.63 (m, 3H), 4.08 (dd, 1H, J=8.7, 5.7 Hz), 4.57 (t, 1H, J=8.7 Hz), 4.78 (dd, 1H, J=8.7, 7.5 Hz), 5.45 (br s, 1H), 6.65 (d, 1H, J=7.8 Hz), 7.23-7.42 (m, 8H), 7.53 (d, 1H, J=8.1 Hz); ¹³C NMR (CDCl₃) δ 22.02, 26.57, 29.20, 30.38, 43.28, 52.80, 52.93, 53.01, 58.57, 59.11, 70.51, 118.85, 126.76, 128.51, 128.96, 129.20, 130.58, 130.73, 134.92, 135.79, 138.05, 140.38, 158.01, 158.06, 158.15, 171.02; ES-MS m/z 531 (M+1). Anal. Calcd. for C₃₀H₃₄N₄SO₃.0.4CH₂Cl₂: C, 64.66; H, 6.21; N, 9.92. Found: C, 64.75; H, 6.22; N, 9.99.

### EXAMPLE 250

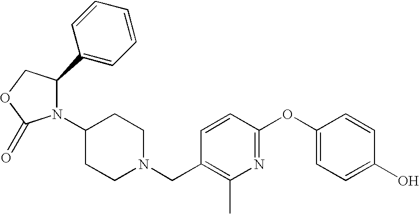

### COMPOUND 250: (R)-3-{1-[6-(4-Hydroxy-phenoxy)-2-methyl-pyridin-3-ylmethyl]-piperidin-4-yl}-4-phenyl-oxazolidin-2-one

A mixture of 6-bromo-2-methylpyridine-3-carboxaldehyde (1.00 g, 5.00 mmol), 4-hydroxy-benzonitrile (0.620 g, 5.00 mmol) and K₂CO₃ (0.414 g, 3.00 mmol) in DMF (10 mL) was stirred at 130° C. for 1 h. Aqueous work-up and purification by flash chromatography on silica gel (EtOAc/hexanes, 1:3 in v/v) afforded 6-(4-methoxy-phenoxy)-2-methyl-pyridine-3-carbaldehyde as a yellow oil (0.966 g, 80%). ¹H NMR (CDCl₃) δ 2.75 (s, 3H), 3.84 (s, 3H), 6.70 (d, 1H, J=8.4 Hz), 6.92-6.97 (m, 2H), 7.06-7.10 (m, 2H), 8.06 (d, 1H, J=8.4 Hz), 10.23 (s, 1H).

Following general procedure A, using (R)-4-phenyl-3-piperidin-4-yl-oxazolidin-2-one (0.230 g, 0.939 mmol), 6-(4-methoxy-phenoxy)-2-methyl-pyridine-3-carbaldehyde (0.285 g, 1.17 mmol), and 2 drops of AcOH. Purification of the crude product by flash chromatography on silica gel (EtOAc) gave (R)-3-{1-[6-(4-methoxy-phenoxy)-2-methyl-pyridin-3-ylmethyl]-piperidin-4-yl}-4-phenyl-oxazolidin-2-one as a colorless oil (0.215 g, 49%).

To a solution of the above substrate (0.185 g, 0.390 mmol) in CH₂Cl₂ (10 mL) was added BBr₃ (1.0 M in CH₂Cl₂, 2.0 mL, 2.0 mmol), and the mixture was stirred at room temperature for 16 h. Methanol (3.0 mL) was added and the mixture was concentrated. The addition and concentration cycle was repeated three times, and then saturated aqueous NaHCO₃ (20 mL) was added. Extraction with CH₂Cl₂ (3×20 mL) was performed and the combined extract was dried over anhydrous Na₂SO₄. After filtration the solvent was removed and the residue was purified by flash chromatography on silica gel (EtOAc) to afford COMPOUND 250 as a white solid (0.130 g, 72%). ¹H NMR (CDCl₃) δ 1.12-1.23 (m, 1H), 1.44-1.48 (m, 1H), 1.72-1.77 (m, 1H), 1.89-2.00 (m, 2H), 2.11 (t, 1H, J=11.1 Hz), 2.43 (s, 3H), 2.68-2.72 (m, 1H), 2.93-2.96 (m, 1H), 3.30 (d, 1H, J=13.2 Hz), 3.40 (d, 1H, J=13.2 Hz), 3.64-3.70 (m, 1H), 4.06-4.08 (m, 1H), 4.56 (t, 1H, J=8.7 Hz), 4.76 (dd, 1H, J=9.0, 5.7 Hz), 6.42 (d, 1H, J=8.4 Hz), 6.88-6.92 (m, 2H), 7.02-7.06 (m, 2H), 7.31-7.41 (m, 6H); ES-MS m/z 482 (M+Na).

### EXAMPLE 251

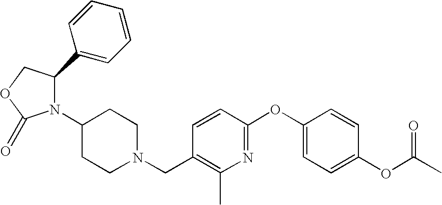

### COMPOUND 251: Acetic acid 4-{6-methyl-5-[4-((R)-2-oxo-4-phenyl-oxazolidin-3-yl)-piperidin-1-ylmethyl]-pyridin-2-yloxy}-phenyl ester

To a solution of (R)-3-{1-[6-(4-hydroxy-phenoxy)-2-methyl-pyridin-3-ylmethyl]-piperidin-4-yl}-4-phenyl-oxazolidin-2-one (COMPOUND 250) (33.3 mg, 0.0725 mmol) in dichloromethane (1.0 mL) at 0° C. was added acetyl chloride (5.4 μL). Standard work-up followed by purification by preparative TLC, eluted with 10% methanol in dichloromethane, provided COMPOUND 251 (24.4 mg, 67%). ¹H NMR (CDCl₃) δ 1.15-1.28 (m, 1H), 1.48 (d, 1H, J=12.0 Hz), 1.71 (d, 1H, J=12.9 Hz), 1.85-2.04 (m, 3H), 2.30 (s, 3H), 2.38 (s, 3H), 2.66 (d, 1H, J=10.8 Hz), 2.83 (d, 1H, J=9.9 Hz), 3.30 (br s, 2H), 3.60 (tt, 1H, J=12.0, 3.9 Hz), 4.08 (dd, 1H, J=8.7, 5.7 Hz), 4.57 (t, 1H, J=8.7 Hz), 4.79 (dd, 1H, J=9.0, 5.7 Hz), 6.52 (d, 1H, J=8.4 Hz), 7.05-7.12 (m, 4H), 7.31-7.44 (m, 6H); ¹³C NMR (CDCl₃) δ 21.51, 22.24, 29.62, 30.85, 53.18, 53.30, 53.83, 58.89, 59.23, 70.90, 107.75, 121.85, 122.94, 127.15, 129.32, 129.58, 140.90, 141.26, 147.25, 152.51, 156.97, 158.45, 162.11, 169.92; ES-MS m/z 502 (M+H). Anal. Calcd. for C₂₉H₃₁N₃O₅.0.2CH₂Cl₂: C, 67.63; H, 6.10; N, 8.10. Found: C, 67.75; H, 6.17; N, 8.18.

### EXAMPLE 252

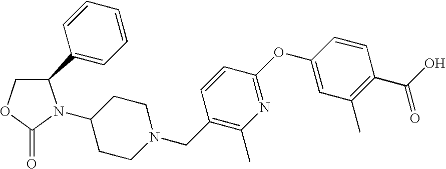

### COMPOUND 252: 2-Methyl-4-{6-methyl-5-[4-((R)-2-oxo-4-phenyl-oxazolidin-3-yl)-piperidin-1-ylmethyl]-pyridin-2-yloxy}-benzoic acid

Following general procedure A, (R)-4-phenyl-3-piperidin-4-yl-oxazolidin-2-one (104 mg, 0.422 mmol) and 4-(5-formyl-6-methyl-pyridin-2-yloxy)-2-methyl-benzonitrile (see EXAMPLE 103) (107 mg, 0.424 mmol) afforded 2-methyl-4-{6-methyl-5-[4-((R)-2-oxo-4-phenyl-oxazolidin-3-yl)-piperidin-1-ylmethyl]-pyridin-2-yloxy}-benzonitrile (74 mg, 36%) following work-up and purification.

Following general procedure I, the above nitrile (74 mg, 0.15 mmol) afforded 4-{5-[4-((R)-2-hydroxy-1-phenyl-ethylamino)-piperidin-1-ylmethyl]-6-methyl-pyridin-2-yloxy}-2-methyl-benzoic acid as a colourless foam (73 mg, 71%).

To the above acid (52 mg, 0.10 mmol) in 10% MeOH/CH₂Cl₂ (2 mL) was added freshly prepared CH₂N₂ in ether until the yellow colour persisted. The mixture was stirred overnight to afford the crude methyl ester. Following general procedure K: to a solution of the above methyl ester (64 mg) and Et₃N (0.036 mL, 0.26 mmol) in CH₂Cl₂ (2.6 mL) at 0° C. was added triphosgene (19 mg, 0.064 mmol) in CH₂Cl₂ (0.5 mL) and the mixture was stirred at room temperature for 1.5 hours. Standard work-up and purification afforded 2-methyl-4-{6-methyl-5-[4-((R)-2-oxo-4-phenyl-oxazolidin-3-yl)-piperidin-1-ylmethyl]-pyridin-2-yloxy}-benzoic acid methyl ester as a yellow oil (42 mg, 53% over 2 steps).

Following general procedure H, the above methyl ester (42 mg, 0.081 mmol) afforded COMPOUND 252 as a colourless foam (28 mg, 68%). ¹H NMR (CD₃OD) δ 1.46 (dd, 1H, J=24.9, 12.6, 4.2 Hz), 1.60-1.64 (m, 1H), 1.71-1.75 (m, 1H), 2.01-2.23 (m, 3H), 2.40 (s, 3H), 2.55 (s, 3H), 2.83-2.87 (m, 1H), 2.96-2.99 (m, 1H), 3.43-3.54 (m, 3H), 4.11 (dd, 1H, J=8.7, 5.7 Hz), 4.64 (t, 1H, J=9.0 Hz), 4.98 (dd, 1H, J=9.0, 5.7 Hz), 6.70 (d, 1H, J=8.1 Hz), 6.88-6.94 (m, 2H), 7.34-7.45 (m, 5H), 7.65 (d, 1H, J=8.4 Hz), 7.90 (d, 1H, J=8.4 Hz); ¹³C NMR (CDCl₃) δ 22.32, 22.75, 28.99, 30.30, 53.05, 58.71, 58.80, 70.99, 109.11, 117.43, 123.25, 126.56, 127.13, 129.34, 129.58, 133.76, 140.71, 142.14, 143.95, 157.23, 158.36, 158.54, 161.51; ES-MS m/z 502 (M+1). Anal. Calcd. for C₂₉H₃₁N₃O₅.0.1CH₂Cl₂: C, 68.52; H, 6.17; N, 8.24. Found: C, 68.44; H, 6.23; N, 8.06.

### EXAMPLE 253

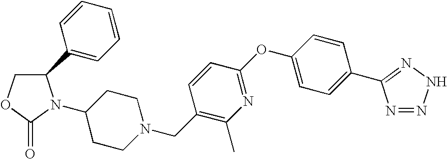

### COMPOUND 253: (R)-3-(1-{2-Methyl-6-[4-(2H-tetrazol-5-yl)-phenoxy]-pyridin-3-ylmethyl}-piperidin-4-yl)-4-phenyl-oxazolidin-2-one

Following general procedure A, (R)-4-phenyl-3-piperidin-4-yl-oxazolidin-2-one (92 mg, 0.37 mmol) and 4-(5-formyl-6-methyl-pyridin-2-yloxy)-benzonitrile (89 mg, 0.37 mmol) afforded 4-{6-methyl-5-[4-((R)-2-oxo-4-phenyl-oxazolidin-3-yl)-piperidin-1-ylmethyl]-pyridin-2-yloxy}-benzonitrile (114 mg, 65%).

A solution of the above nitrile (114 mg, 0.243 mmol), NH₄Cl (52.8 mg, 0.949 mmol) and NaN₃ (47 mg, 0.72 mmol) in DMF (3 mL) was heated to 100° C. overnight. Standard work-up and purification afforded COMPOUND 253 as a white solid (42 mg, 34%). ¹H NMR (CD₃OD) δ 1.76-1.88 (m, 3H), 2.23-2.35 (m, 1H), 2.41 (s, 3H), 2.73-2.83 (m, 2H), 3.24-3.36 (m, 1H), 3.54-3.63 (m, 1H), 4.02 (s, 2H), 4.06-4.14 (m, 2h), 4.63-4.69 (m, 1H), 4.97 (dd, 1H, J=8.7, 6.0 Hz), 6.77 (d, 1H, J=8.4 Hz), 7.20 (d, 2H, J=8.4 Hz), 7.35-7.40 (m, 5H), 7.71 (d, 1H, J=8.4 Hz), 8.03 (d, 2H, J=8.7 Hz); ¹³C NMR (CD₃OD) δ 20.14, 26.33, 26.90, 50.26, 51.36, 53.01, 56.28, 58.86, 70.36, 108.28, 120.49, 124.30, 126.45, 127.64, 128.39, 128.62, 139.24, 142.92, 154.85, 157.29, 158.25, 162.40; ES-MS m/z 512 (M+1).

### EXAMPLE 254

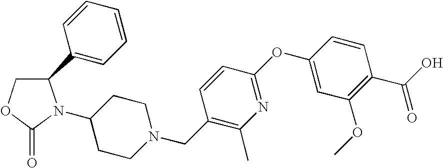

### COMPOUND 254: 2-Methoxy-4-{6-methyl-5-[4-((R)-2-oxo-4-phenyl-oxazolidin-3-yl)-piperidin-1-ylmethyl]-pyridin-2-yloxy}-benzoic acid

A solution of 2-fluoro-4-hydroxybenzonitrile (308 mg, 2.25 mmol), 6-bromo-2-methyl-pyridine-3-carbaldehyde (449 mg, 2.24 mmol) and K₂CO₃ (310 mg, 2.24 mmol) in DMF (4.5 mL) was heated to 140° C. for 20 minutes. Aqueous work-up and purification afforded 2-fluoro-4-(5-formyl-6-methyl-pyridin-2-yloxy)-benzonitrile (122 mg, 21%).

COMPOUND 254 was prepared using the same chemistry as for COMPOUND 252 except that 2-fluoro-4-(5-formyl-6-methyl-pyridin-2-yloxy)-benzonitrile was used in lieu of 4-(5-formyl-6-methyl-pyridin-2-yloxy)-2-methyl-benzonitrile. COMPOUND 254 was isolated as a yellow foam. ¹H NMR (CD₃OD) δ 1.48 (ddd, 1H, J=24.6, 12.3, 3.9 Hz), 1.61-1.65 (m, 1H), 1.73-1.77 (m, 1H), 2.03-2.28 (m, 3H), 2.41 (s, 3H), 2.86-2.90 (m, 1H), 2.99-3.03 (m, 1H), 3.45-3.57 (m, 3H), 3.85 (s, 3H), 4.11 (dd, 1H, J=8.4, 5.7 Hz), 4.65 (t, 1H, J=9.0 Hz), 4.98 (dd, 1H, J=9.0, 6.0 Hz), 6.64 (dd, 1H, J=8.4, 1.8 Hz), 6.75 (d, 1H, J=8.4 Hz), 6.85 (d, 1H, J=1.8 Hz), 7.36-7.44 (m, 5H), 7.68 (d, 1H, J=8.4 Hz), 7.81 (d, 1H, J=8.7 Hz); ES-MS m/z 518 (M+1).

### EXAMPLE 255

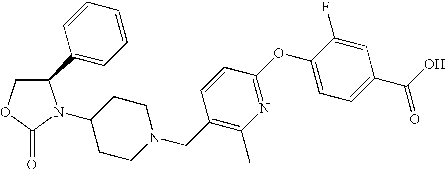

### COMPOUND 255: 3-Fluoro-4-{6-methyl-5-[4-((R)-2-oxo-4-phenyl-oxazolidin-3-yl)-piperidin-1-ylmethyl]-pyridin-2-yloxy}-benzoic acid

COMPOUND 255 was prepared using the same chemistry as for COMPOUND 252 except that 4-bromo-2-fluorophenol was used in lieu of 4-bromo-3-methylphenol. COMPOUND 255 was isolated as a yellow foam. ¹H NMR (CD₃OD) δ 1.56-1.83 (m, 3H), 2.20 (ddd, 1H, J=24.9, 12.6, 3.9 Hz), 2.35 (s, 3H), 2.41-2.54 (m, 2H), 3.05-3.09 (m, 1H), 3.15-3.18 (m, 1H), 3.44-3.57 (m, 1H), 3.79 (s, 2H), 4.11 (dd, 1H, J=8.7, 6.0 Hz), 4.66 (t, 1H, J=8.7 Hz), 4.99 (dd, 1H, J=8.7, 6.0 Hz), 6.84 (d, 1H, J=8.4 Hz), 7.23-7.29 (m, 1H), 7.35-7.40 (m, 5H), 7.72 (d, 1H, J=8.1 Hz), 7.78-7.86 (m, 2H); ¹³C NMR (CD₃OD) δ 22.39, 28.81, 29.57, 52.92, 53.51, 53.69, 58.53, 60.78, 72.60, 109.55, 119.08, 119.34, 123.86, 124.77, 127.78, 128.61, 130.51, 130.78, 141.74, 144.91, 154.22, 157.51, 159.01, 160.52, 163.55; ES-MS m/z 506 (M+1). Anal. Calcd. for C₂₈H₂₈N₃FO₅.0.1CH₂Cl₂.CH₄O: C, 64.32; H, 5.86; N, 7.79. Found: C, 64.05; H, 5.80; N, 7.59.

### EXAMPLE 256

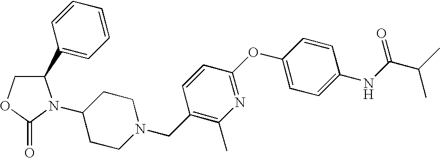

### COMPOUND 256: N-(4-{6-Methyl-5-[4-((R)-2-oxo-4-phenyl-oxazolidin-3-yl)-piperidin-1-ylmethyl]-pyridin-2-yloxy}-phenyl)-isobutyramide

A solution of 4-nitrophenol (241 mg, 1.73 mmol), 6-bromo-2-methyl-pyridine-3-carbaldehyde (415 mg, 2.07 mmol) and K₂CO₃ (239 mg, 1.73 mmol) in DMF (3.5 mL) was heated to 130° C. for 1 hour. Aqueous work-up and purification afforded 2-methyl-6-(4-nitro-phenoxy)-pyridine-3-carbaldehyde (162 mg). Following general procedure A, (R)-4-phenyl-3-piperidin-4-yl-oxazolidin-2-one (155 mg, 0.629 mmol) and the above aldehyde afforded (R)-3-{1-[2-methyl-6-(4-nitro-phenoxy)-pyridin-3-ylmethyl]-piperidin-4-yl}-4-phenyl-oxazolidin-2-one (165 mg) which was subsequently hydrogenated in MeOH over 10% Pd/C at 45 psi for 1.5 hours. Filtration and purification afforded (R)-3-{1-[6-(4-amino-phenoxy)-2-methyl-pyridin-3-ylmethyl]-piperidin-4-yl}-4-phenyl-oxazolidin-2-one (89 mg). To a solution of the above amine and Et₃N (0.027 mL, 0.19 mmol) in CH₂Cl₂ (1.9 mL) at −25° C. was added isobutyryl chloride (0.012 mL, 0.11 mmol) in CH₂Cl₂ (2 mL). The mixture was allowed to warm to room temperature overnight. Aqueous work-up and purification afforded COMPOUND 256 as a yellow foam (26 mg, 5% over 4 steps). ¹H NMR (CDCl₃) δ 1.14-1.28 (m, 7H), 1.43-1.49 (m, 1H), 1.69-1.73 (m, 1H), 1.85-2.03 (m, 3H), 2.37 (s, 3H), 2.42-2.58 (m, 1H), 2.63-2.67 (m, 1H), 2.81-2.84 (m, 1H), 2.37 (s, 3H), 2.51 (septet, 1H, J=6.9 Hz), 2.63-2.74 (m, 1H), 2.81-2.84 (m, 1H), 3.25-3.34 (m, 2H), 3.56-3.65 (m, 1H), 4.09 (dd, 1H, J=8.4, 5.7 Hz), 4.57 (t, 1H, J=8.7 Hz), 4.79 (dd, 1H, J=8.7, 5.7 Hz), 6.48 (d, 1H, J=8.1 Hz), 7.06 (d, 2H, J=9.0 Hz), 7.21 (br s, 1H), 7.31-7.42 (m, 6H), 7.52 (d, 1H, J=8.7 Hz); ¹³C NMR (CDCl₃) δ 19.65, 21.85, 29.24, 30.45, 36.55, 52.77, 52.90, 53.10, 58.53, 58.86, 70.54, 107.01, 121.24, 126.50, 128.95, 129.20, 134.58, 140.45, 140.85, 150.70, 156.54, 158.11, 162.08, 175.30; ES-MS m/z 5516 (M+Na). Anal. Calcd. for C₃₁H₃₆N₄O₄.0.1CH₂Cl₂: C, 69.54; H, 6.79; N, 10.43. Found: C, 69.55; H, 6.96; N, 10.22.

### EXAMPLE 257

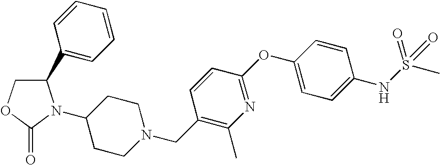

### COMPOUND 257: N-(4-{6-Methyl-5-[4-((R)-2-oxo-4-phenyl-oxazolidin-3-yl)-piperidin-1-ylmethyl]-pyridin-2-yloxy}-phenyl)-methanesulfonamide

To a solution of (R)-3-{1-[6-(4-amino-phenoxy)-2-methyl-pyridin-3-ylmethyl]-piperidin-4-yl}-4-phenyl-oxazolidin-2-one (see EXAMPLE 256) (97 mg, 0.21 mmol) and Et₃N (0.027 mL, 0.19 mmol) in CH₂Cl₂ (4 mL) at −25° C. was added MsCl (0.010 mL, 0.13 mmol) in CH₂Cl₂ (2 mL) dropwise. The mixture was allowed to warm to room temperature overnight. Aqueous work-up and purification afforded COMPOUND 257 as a colourless foam (17 mg, 25%). ¹H NMR (CDCl₃) δ 1.25 (ddd, 1H, J=24.3, 12.0, 3.9 Hz), 1.47-1.51 (m, 1H), 1.69-1.73 (m, 1H), 1.84-2.05 (m, 3H), 2.38 (s, 3H), 2.65-2.68 (m, 1H), 2.82-2.85 (m, 1H), 3.02 (s, 3H), 3.31 (s, 2H), 3.53-3.64 (m, 1H), 4.09 (dd, 1H, J=8.7, 5.7 Hz), 4.58 (t, 1H, J=9.0 Hz), 4.79 (dd, 1H, J=9.0, 5.7 Hz), 6.56 (d, 2H, J=8.1 Hz), 7.08-7.13 (m, 2H), 7.22-7.26 (m, 2H), 7.31-7.43 (m, 5H), 7.46 (d, 1H, J=8.4 Hz); ¹³C NMR (CDCl₃) δ 21.83, 29.23, 30.38, 39.20, 52.82, 52.92, 53.08, 58.62, 58.84, 70.56, 107.67, 121.65, 123.22, 126.78, 127.02, 128.96, 129.21, 132.65, 140.40, 140.99, 152.52, 156.56, 158.15, 161.45; ES-MS m/z 537 (M+1). Anal. Calcd. for C₂₈H₃₂N₄SO₅.0.3CH₂Cl₂: C, 60.47; H, 5.85; N, 9.97. Found: C, 60.70; H, 6.07; N, 9.68.

### EXAMPLE 258

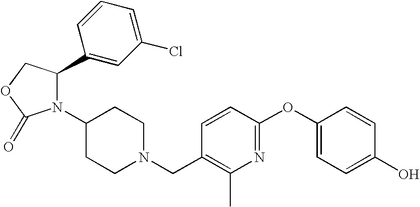

### COMPOUND 258: (R)-4-(3-Chloro-phenyl)-3-{1-[6-(4-hydroxy-phenoxy)-2-methyl-pyridin-3-ylmethyl]-piperidin-4-yl}-oxazolidin-2-one

To a solution of (R)-4-(3-chloro-phenyl)-3-{1-[6-(4-methoxy-phenoxy)-2-methyl-pyridin-3-ylmethyl]-piperidin-4-yl}-oxazolidin-2-one (COMPOUND 221) (150.0 mg, 0.296 mmol) in CH₂Cl₂ (5.0 mL) was added a solution of BBr₃ in CH₂Cl₂ (1 M, 1.48 mL, 1.48 mmol) at 0° C. The mixture was stirred at rt for 17 h. MeOH (3.0 mL) was added to the mixture. The mixture was concentrated in vacuo. Standard work-up and purification gave COMPOUND 258 (91.8 mg, 63%). ¹H NMR (CDCl₃) δ 1.16-1.30 (m, 1H), 1.50 (d, 1H, J=12.8 Hz), 1.75 (d, 1H, J=11.4 Hz), 1.90-2.00 (m, 2H), 2.13 (t, 1H, J=11.4 Hz), 2.44 (s, 3H), 2.75 (d, 1H, J=10.5 Hz), 2.96 (d, 1H, J=10.5 Hz), 3.33 (d, 1H, J=13.2 Hz), 3.41 (d, 1H, J=13.2 Hz), 3.71 (m, 1H), 4.03 (dd, 1H, J=8.4, 5.4 Hz), 4.55 (t, 1H, J=8.7 Hz), 4.71 (dd, 1H, J=9, 6 Hz), 6.25 (d, 1H, J=8.4 Hz), 6.60 (d, 2H, J=8.4 Hz), 6.85 (d, 2H, J=8.4 Hz), 7.04 (d, 1H, J=7.5 Hz), 7.13 (t, 1H, J=7.5 Hz), 7.19-7.26 (m, 2H), 7.42 (d, 1H, J=8.4 Hz), 8.33 (br s, 1H); ¹³C NMR (CDCl₃) δ 22.2, 29.1, 30.7, 52.8, 53.2, 53.3, 58.1, 58.8, 70.8, 106.6, 117.0, 122.5, 125.2, 125.4, 127.0, 129.5, 130.9, 135.5, 142.2, 142.9, 147.0, 154.1, 157.0, 158.5, 163.7; ES-MS m/z 494 (M+H). Anal. Calcd. for C₂₇H₂₈N₃O₄Cl.0.9CH₂Cl₂: C, 58.75; H, 5.27; N, 7.37. Found: C, 58.62; H, 5.42; N, 7.03.

### EXAMPLE 259

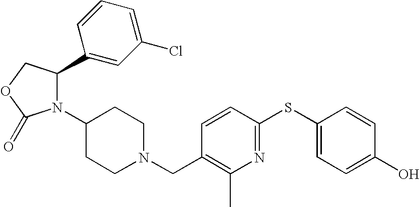

### COMPOUND 259: (R)-4-(3-Chloro-phenyl)-3-{1-[6-(4-hydroxy-phenylsulfanyl)-2-methyl-pyridin-3-ylmethyl]-piperidin-4-yl}-oxazolidin-2-one

To a solution of (R)-4-(3-chloro-phenyl)-3-{1-[6-(4-methoxy-phenylsulfanyl)-2-methyl-pyridin-3-ylmethyl]-piperidin-4-yl}-oxazolidin-2-one (COMPOUND 220) (130.0 mg, 0.248 mmol) in CH₂Cl₂ (5.0 mL) was added a solution of BBr₃ in CH₂Cl₂ (1 M, 1.24 mL, 1.24 mmol) at 0° C. The mixture was stirred at rt for 17 h. MeOH (3.0 mL) was added to the mixture. The mixture was concentrated in vacuo. Standard work-up and purification gave COMPOUND 259 (90.1 mg, 71%). ¹H NMR (CDCl₃) δ 1.17-1.30 (m, 1H), 1.49 (d, 1H, J=11.1 Hz), 1.74 (d, 1H, J=10.2 Hz), 1.86-2.00 (m, 2H), 2.10 (t, 1H, J=11.1 Hz), 2.48 (s, 3H), 2.74 (d, 1H, J=10.5 Hz), 2.92 (d, 1H, J=10.8 Hz), 3.32 (d, 1H, J=13.5 Hz), 3.38 (d, 1H, J=13.5 Hz), 3.67 (m, 1H), 4.02 (dd, 1H, J=8.4, 5.4 Hz), 4.55 (t, 1H, J=8.7 Hz), 4.67 (dd, 1H, J=9.0, 5.1 Hz), 6.41 (d, 1H, J=8.1 Hz), 6.71 (d, 2H, J=8.4 Hz), 7.03 (d, 1H, J=7.5 Hz), 7.15-7.29 (m, 4H), 7.36 (d, 2H, J=7.8 Hz); ¹³C NMR (CDCl₃) δ 20.7, 27.8, 29.3, 51.6, 51.8, 51.9, 56.8, 57.7, 69.4, 116.2, 116.5, 118.2, 123.7, 125.6, 125.7, 128.2, 129.6, 134.1, 136.6, 137.8, 141.4, 156.4, 157.2, 157.4, 160.5; ES-MS m/z 510 (M+H). Anal. Calcd. for C₂₇H₂₈N₃O₃SCl.0.4CH₂Cl₂: C, 60.49; H, 5.34; N, 7.72. Found: C, 60.56; H, 5.50; N, 7.38.

### EXAMPLE 260

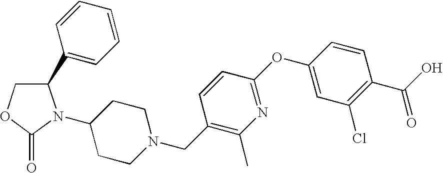

### COMPOUND 260: 2-Chloro-4-{6-methyl-5-[4-((R)-2-oxo-4-phenyl-oxazolidin-3-yl)-piperidin-1-ylmethyl]-pyridin-2-yloxy}-benzoic acid

A solution of 2-chloro-4-hydroxybenzonitrile (257 mg, 1.67 mmol), 6-bromo-2-methyl-pyridine-3-carbaldehyde (402 mg, 1.17 mmol) and K₂CO₃ (162 mg, 1.17 mmol) in DMF (3.3 mL) was heated to 130° C. for 1.25 hours. Aqueous work-up and purification afforded 2-chloro-4-(5-formyl-6-methyl-pyridin-2-yloxy)-benzonitrile (145 mg).

COMPOUND 260 was prepared using the same chemistry as COMPOUND 252 except that 2-chloro-4-(5-formyl-6-methyl-pyridin-2-yloxy)-benzonitrile was used in lieu of 4-(5-formyl-6-methyl-pyridin-2-yloxy)-2-methyl-benzonitrile. COMPOUND 260 was isolated as a colourless foam. ¹H NMR (CD₃OD) δ 1.67-1.89 (m, 3H), 2.23-2.35 (m, 1H), 2.45 (s, 3H), 2.58-2.68 (m, 2H), 3.15-3.29 (m, 1H), 3.54-3.64 (m, 1H), 3.92 (s, 2H), 4.16 (dd, 1H, J=8.7, 6.0 Hz), 4.70 (t, 1H, J=8.7 Hz), 5.03 (dd, 1H, J=9.0, 6.0 Hz), 6.86 (d, 1H, J=8.1 Hz), 7.08-7.10 (m, 1H), 7.21 (s, 1H), 7.39-7.45 (m, 5H), 7.76-7.81 (m, 2H); ES-MS m/z 522 (M+1). Anal. Calcd. for C₂₈H₂₈N₃ClO₅.0.6CH₂Cl₂.0.8CH₄O: C, 58.99; H, 5.46; N, 7.02. Found: C, 59.15; H, 5.51; N, 7.12.

### EXAMPLE 261

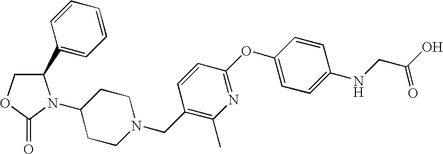

### COMPOUND 261: (4-{6-Methyl-5-[4-((R)-2-oxo-4-phenyl-oxazolidin-3-yl)-piperidin-1-ylmethyl]-pyridin-2-yloxy}-phenylamino)-acetic acid

Following general procedure G: a solution of (R)-3-{1-[6-(4-amino-phenoxy)-2-methyl-pyridin-3-ylmethyl]-piperidin-4-yl}-4-phenyl-oxazolidin-2-one (see EXAMPLE 256) (109 mg, 0.238 mmol), methyl bromoacetate (0.016 mL, 0.17 mmol) and DIPEA (0.041 mL, 0.24 mmol) in CH₃CN (2.4 mL) was heated to 60° C. for 25 hours. Purification afforded (4-{6-methyl-5-[4-((R)-2-oxo-4-phenyl-oxazolidin-3-yl)-piperidin-1-ylmethyl]-pyridin-2-yloxy}-phenylamino)-acetic acid methyl ester as a colourless foam (54 mg, 60%).

Following general procedure H, the above methyl ester (35 mg, 0.066 mmol afforded COMPOUND 261 as a yellow foam (26 mg, 76%). ¹H NMR (CD₃OD) δ 1.61-1.86 (m, 3H), 2.24 (ddd, 1H, J=24.6, 12.0, 3.6 Hz), 2.46 (s, 3H), 2.50-2.60 (m, 2H), 3.09-3.12 (m, 1H), 3.18-3.22 (m, 1H), 3.52-3.60 (m, 1H), 3.83 (br s, 4H), 4.15 (dd, 1H, J=8.7, 6.0 Hz), 4.69 (t, 1H, J=9.0 Hz), 5.02 (dd, 1H, J=8.7, 6.0 Hz), 6.54 (d, 1H, J=8.4 Hz), 6.67-6.70 (m, 2H), 6.92 (d, 2H, J=8.1 Hz), 7.39-7.49 (m, 5H), 7.64 (d, 1H, J=8.7 Hz); ¹³C NMR (CD₃OD) δ 22.31, 28.78, 29.43, 52.84, 53.48, 53.62, 58.53, 60.93, 72.61, 108.61, 115.34, 122.45, 123.31, 128.69, 130.57, 130.84, 141.76, 144.90, 146.75, 147.86, 159.13, 160.51, 166.19; ES-MS m/z 517 (M+1). Anal. Calcd. for C₂₉H₃₂N₄O₅.0.5CH₂Cl₂: C, 63.38; H, 5.95; N, 10.02. Found: C, 63.51; H, 6.11; N, 10.05.

### EXAMPLE 262

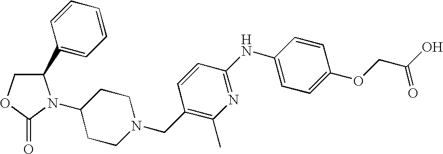

### COMPOUND 262: (4-{6-Methyl-5-[4-((R)-2-oxo-4-phenyl-oxazolidin-3-yl)-piperidin-1-ylmethyl]-pyridin-2-ylamino}-phenoxy)-acetic acid

Following general procedure A, (R)-4-phenyl-3-piperidin-4-yl-oxazolidin-2-one (282 mg, 1.14 mmol) and 6-bromo-2-methyl-pyridine-3-carbaldehyde (229 mg, 1.14 mmol) afforded (R)-3-[1-(6-bromo-2-methyl-pyridin-3-ylmethyl)-piperidin-4-yl]-4-phenyl-oxazolidin-2-one as a colourless solid (235 mg, 48%).

A solution of 4-nitrophenol (554 mg, 3.98 mmol), tert-butyl bromoacetate (0.71 mL, 4.8 mmol) and K₂CO₃ (550 mg, 3.98 mmol) in CH₃CN (20 mL) was heated at 70° C. for 1 hour. The mixture was filtered and concentrated under reduced pressure to afford the crude nitro compound which was subsequently hydrogenated in MeOH (10 mL) over 10% Pd/C (150 mg) at 45 psi for 1.5 hours. Filtration and purification afforded (4-amino-phenoxy)-acetic acid tert-butyl ester as a yellow oil (620 mg, 70% over 2 steps).

A mixture of the above bromide (235 mg, 0.546 mmol), the above amine (305 mg, 1.37 mmol), Pd₂(dba)₃ (50 mg, 0.055 mmol), DPPF (61 mg, 0.11 mmol) and Cs₂CO₃ (267 mg, 0.819 mmol) in degassed dioxane (1.1 mL) was heated to 120° C. under Ar for 18 hours. Filtration and purification afforded (4-{6-methyl-5-[4-((R)-2-oxo-4-phenyl-oxazolidin-3-yl)-piperidin-1-ylmethyl]-pyridin-2-ylamino}-phenoxy)-acetic acid tert-butyl ester (254 mg, 81%).

A solution of the above substrate (236 mg, 0.412 mmol) was stirred in 1:1 TFA/CH₂Cl₂ (6 mL) at room temperature 3 hours. Standard work-up and purification afforded COMPOUND 262 as a yellow foam (172 mg, 81%). ¹H NMR (CD₃OD) δ 1.93-1.96 (m, 3H), 2.34-2.47 (m, 4H), 2.96-3.03 (m, 2H), 3.39-3.49 (m, 2H), 3.52-3.68 (m, 1H), 3.38-3.49 (m, 2H), 3.56-3.68 (m, 1H), 4.13 (s, 2H), 4.17 (dd, 1H, J=8.1, 6.0 Hz), 4.56 (s, 2H), 4.71 (t, 1H, J=8.7 Hz), 5.03 (dd, 1H, J=8.7, 6.0 Hz), 6.61 (d, 1H, J=8.7 Hz), 6.90-6.93 (m, 2H), 7.42-7.50 (m, 8H); ¹³C NMR (CD₃OD) δ 20.82, 26.49, 26.92, 50.38, 51.47, 51.56, 57.04, 59.71, 66.52, 71.22, 107.31, 112.87, 115.25, 122.48, 127.32, 129.26, 126.52, 134.11, 140.02, 142.08, 154.77, 156.78, 156.94, 158.98; ES-MS m/z 517 (M+1). Anal. Calcd. for C₂₉H₃₂N₄O₅.1.5CH₂Cl₂: C, 56.88; H, 5.48; N, 8.70. Found: C, 56.96; H, 5.54; N, 8.66.

### EXAMPLE 263

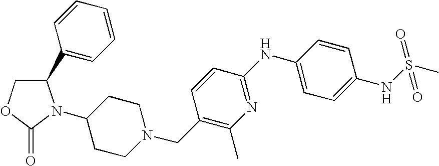

### COMPOUND 263: N-(4-{6-Methyl-5-[4-((R)-2-oxo-4-phenyl-oxazolidin-3-yl)-piperidin-1-ylmethyl]-pyridin-2-ylamino}-phenyl)-methanesulfonamide

A solution of 4-nitroaniline (937 mg, 6.78 mmol), BOC₂O (3.7 g, 17 mmol), DMAP (20 mg, 0.16 mmol) and Et₃N (1.0 mL, 7.2 mmol) in THF was heated to 80° C. for 20 hours. The mixture concentrated under reduced pressure and purified to afford the desired compound as yellow crystals (2.12 g, 92%). The above substrate (2.12 g, 6.27 mmol) was subsequently hydrogenated in MeOH (25 mL) over 10% Pd/C (300 mg) at 45 psi for 2 hours. Filtration afforded the aniline as redish-tan crystals (1.88 g, 97%).

To a solution of the above aniline (921 mg, 2.99 mmol) and Et₃N (0.42 mL, 3.0 mmol) in CH₂Cl₂ (60 mL) at −25° C. was added MsCl (0.16 mL, 2.1 mmol) in CH₂Cl₂ (8 mL) and the mixture was stirred at room temperature 3.5 hours. Standard aqueous work-up and purification afforded the methylsulfonamide. The above substrate (655 mg) in 1:1 TFA/CH₂Cl₂ (10 mL) was stirred at room temperature for 15 minutes. Aqueous work-up and purification afforded N-(4-amino-phenyl)-methanesulfonamide as a yellow solid (240 mg, 62%).

A mixture of (R)-3-[1-(6-bromo-2-methyl-pyridin-3-ylmethyl)-piperidin-4-yl]-4-phenyl-oxazolidin-2-one (see EXAMPLE 261) (222 mg, 0.516 mmol), the above amine (240 mg, 1.29 mmol), Pd₂(dba)₃ (47 mg, 0.051 mmol), DPPF (57 mg, 0.10 mmol) and Cs₂CO₃ (252 mg, 0.773 mmol) in degassed dioxane (1.0 mL) was heated to 120° C. under Ar for 17 hours. Filtration and purification afforded COMPOUND 263 as a yellow foam (68 mg, 25%). ¹H NMR (CDCl₃) δ 1.17-1.30 (m, 1H), 1.46-1.50 (m, 1H), 1.69-1.73 (m, 1H), 1.83-2.03 (m, 3H), 2.39 (s, 3H), 2.66-2.70 (m, 1H), 2.84-2.86 (m, 1H), 2.99 (s, 3H), 3.22-3.32 (m, 2H), 3.54-3.62 (m, 1H), 4.09 (dd, 1H, J=8.4, 5.7 Hz), 4.57 (t, 1H, J=9.0 Hz), 4.79 (dd, 1H, J=8.7, 5.7 Hz), 6.36 (br s, 1H), 6.46 (br s, 1H), 6.61 (d, 1H, J=8.1 Hz), 7.17-7.20 (m, 2H), 7.27-7.42 (m, 8H); ¹³C NMR (CDCl₃) δ 21.89, 29.25, 30.37, 39.05, 52.70, 52.80, 53.17, 58.65, 59.15, 70.55, 105.41, 120.38, 123.06, 123.82, 126.78, 128.93, 129.19, 130.41, 139.21, 139.66, 140.41, 153.75, 156.46, 158.15; ES-MS m/z 536 (M+1). Anal. Calcd. for C₂₈H₃₃N₅SO₄.0.4CH₂Cl₂.0.1C₆H,₄: C, 60.24; H, 6.14; N, 12.11. Found: C, 60.44; H, 6.01; N, 11.93.

### EXAMPLE 264

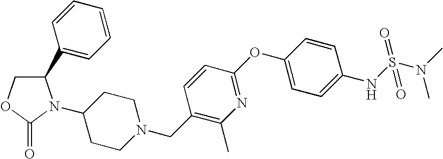

### COMPOUND 264: N,N-Dimethyl-N′-(2-methyl-4-{6-methyl-5-[4-((R)-2-oxo-4-phenyl-oxazolidin-3-yl)-piperidin-1-ylmethyl]-pyridin-2-yloxy}-phenyl)-sulfamide

To a solution of (R)-3-{1-[6-(4-amino-phenoxy)-2-methyl-pyridin-3-ylmethyl]-piperidin-4-yl}-4-phenyl-oxazolidin-2-one (see EXAMPLE 256) (103 mg, 0.225 mmol) and Et₃N (0.038 mL, 0.27 mmol) in CH₂Cl₂ (4 mL) at 0° C. was added dimethylsulfamoyl chloride (0.026 mL, 0.24 mmol) in CH₂Cl₂ (0.5 mL). The mixture was stirred at room temperature for 1 hour. A second aliquot of dimethylsulfamoyl chloride (0.05 mL) was added and the mixture heated to 40° C. for 3 hours. Aqueous work-up and purification afforded COMPOUND 264 as a colourless foam (13 mg, 10%). ¹H NMR (CDCl₃) δ 1.24 (ddd, 1H, J=24.3, 12.3,4.2 Hz), 1.47-1.51 (m, 1H), 1.69-1.73 (m, 1H), 1.86-2.04 (m, 3H), 2.37 (s, 3H), 2.64-2.68 (m, 1H), 2.82-2.86 (m, 7H), 3.55-3.64 (m, 1H), 4.09 (dd, 1H, J=8.7, 5.7 Hz), 4.57 (t, 1H, J=9 Hz), 4.79 (dd, 1H, J=9.0, 5.7 Hz), 6.51-3.53 (m, 2H), 7.05-7.08 (m, 2H), 7.18-7.22 (m, 2H), 7.31-7.45 (m, 6H); ¹³C NMR (CDCl₃) δ 22.23, 29.62, 30.79, 38.64, 53.20, 53.30, 53.47, 58.99, 59.24, 70.92, 107.83, 121.87, 122.87, 127.16, 129.33, 129.59, 133.68, 140.83, 141.30, 152.18, 156.92, 158.79, 162.03; ES-MS m/z 566 (M+1). Anal. Calcd. for C₂₉H₃₅N₅SO₅.0.1CH₂Cl₂.0.3CH₄O: C, 60.49; H, 6.28; N, 12.00. Found: C, 60.65; H, 6.12; N, 11.79.

### EXAMPLE 265

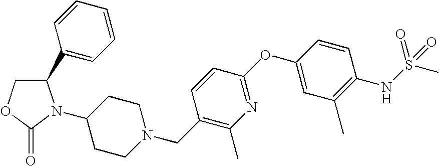

### COMPOUND 265: N-(2-Methyl-4-{6-methyl-5-[4-((R)-2-oxo-4-phenyl-oxazolidin-3-yl)-piperidin-1-ylmethyl]-pyridin-2-yloxy}-phenyl)-methanesulfonamide

COMPOUND 265 was prepared using the same chemistry as COMPOUND 257 except that 3-methyl-4-nitrophenol was used in lieu of 4-nitrophenol. COMPOUND 265 was isolated as a yellow foam. ¹H NMR (CDCl₃) δ 1.25 (ddd, 1H, J=24.3, 12.0, 3.9 Hz), 1.47-1.51 (m, 1H), 1.70-1.74 (m, 1H), 1.87-2.05 (m, 3H), 2.33 (s, 3H), 2.38 (s, 3H), 2.65-2.69 (m, 1H), 2.82-2.85 (m, 1H), 3.03 (s, 3H), 3.31 (s, 2H), 3.55-3.64 (m, 1H), 4.09 (dd, 1H, J=8.7, 6.0 Hz), 4.58 (t, 1H, J=8.7 Hz), 4.79 (dd, 1H, J=9.0, 5.7 Hz), 6.13 (br s, 1H), 6.55 (d, 1H, J=8.4 Hz), 6.94-7.00 (m, 2H), 7.31-7.47 (m, 7H); ¹³C NMR (CDCl₃) δ 18.32, 21.84, 29.23, 30.39, 39.87, 52.83, 52.93, 53.08, 58.60, 58.85, 70.53, 107.77, 119.04, 122.84, 125.92, 126.77, 127.03, 128.94, 129.20, 130.44, 134.03, 140.44, 140.93, 153.10, 156.60, 158.09, 161.42; ES-MS m/z 551 (M+1). Anal. Calcd. for C₂₉H₃₄N₄SO₅.0.4CH₂Cl₂: C, 60.40; H, 6.00; N, 9.58. Found: C, 60.10; H, 5.93; N, 9.47.

### EXAMPLE 266

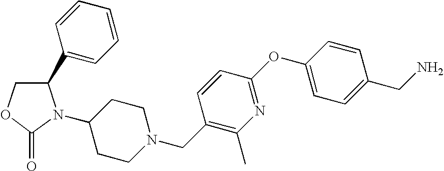

### COMPOUND 266: (R)-3-{1-[6-(4-Aminomethyl-phenoxy)-2-methyl-pyridin-3-ylmethyl]-piperidin-4-yl}-4-phenyl-oxazolidin-2-one

To a solution of 4-{6-methyl-5-[4-((R)-2-oxo-4-phenyl-oxazolidin-3-yl)-piperidin-1-ylmethyl]-pyridin-2-yloxy}-benzoic acid methyl ester (see EXAMPLE 224) (676 mg, 1.35 mmol) in CH₂Cl₂ (6.7 mL) was added DIBAL (1.0M in CH₂Cl₂, 4.0 mL, 4.0 mmol) and the mixture was stirred at room temperature for 20 minutes. The reaction was quenched with 1N NaOH (10 mL) and subsequent work-up and purification afforded (R)-3-{1-[6-(4-hydroxymethyl-phenoxy)-2-methyl-pyridin-3-ylmethyl]-piperidin-4-yl}-4-phenyl-oxazolidin-2-one as a colourless foam (244 mg, 38%).

To a solution of the above alcohol (224 mg, 0.473 mmol) and Et₃N (0.073 mL, 0.52 mmol) in CH₂Cl₂ (4.7 mL) at 0° C. was added MsCl (0.037 mL, 0.48 mmol) and the mixture was stirred at 0° C. for 30 minutes. Aqueous work-up afforded the mesylate (202 mg). To the above mesylate in DMF (4.7 mL) was added NaN₃ (92 mg, 1.4 mmol) and the mixture was stirred at room temperature for 25 hours. Aqueous work-up afforded (R)-3-{I-[6-(4-azidomethyl-phenoxy)-2-methyl-pyridin-3-ylmethyl]-piperidin-4-yl}-4-phenyl-oxazolidin-2-one (174 mg).

To the above azide (174 mg) in THF (3.2 mL) was added Ph₃P (137 mg, 0.522 mmol) and the mixture stirred at room temperature for 23 hours. Standard work-up and purification afforded COMPOUND 266 as a colourless foam (117 mg, 52%). ¹H NMR (CDCl₃) δ 1.22 (ddd, 1H, J=24.3, 12.0, 3.9 Hz), 1.46-1.51 (m, 1H), 1.69-1.73 (m, 1H), 1.84-2.04 (m, 3H), 2.38 (s, 3H), 2.64-2.68 (m, 1H), 2.81-2.85 (m, 1H), 3.30 (s, 2H), 3.56-3.65 (m, 1H), 3.87 (s, 2H), 4.08 (dd, 1H, J=8.4, 5.7 Hz), 4.57 (t, 1H, J=8.7 Hz), 4.79 (dd, 1H, J=9.0, 5.7 Hz); ES-MS m/z 473 (M+1).

### EXAMPLE 267

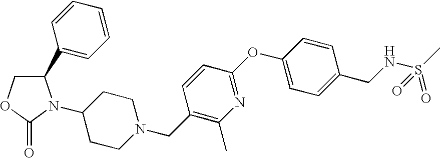

### COMPOUND 267: N-(4-{6-Methyl-5-[4-((R)-2-oxo-4-phenyl-oxazolidin-3-yl)-piperidin-1-ylmethyl]-pyridin-2-yloxy}-benzyl)-methanesulfonamide

To a solution of (R)-3-{1-[6-(4-aminomethyl-phenoxy)-2-methyl-pyridin-3-ylmethyl]-piperidin-4-yl}-4-phenyl-oxazolidin-2-one (COMPOUND 266) (97 mg, 0.21 mmol) and Et₃N (0.029 mL, 0.21 mmol) in CH₂Cl₂ (4 mL) at −25° C. was added MsCl (0.013 mL, 0.17 mmol) in CH₂Cl₂ (2 mL) over 45 minutes. The mixture was warmed to room temperature. Standard work-up and purification afforded COMPOUND 267 as a colourless foam (90 mg, 98%). ¹H NMR (CDCl₃) δ 1.24 (ddd, 1H, J=24.6, 12.3, 3.9 Hz), 1.47-1.51 (m, 1H), 1.61 (s, 3H), 1.69-1.73 (m, 1H), 1.86-2.04 (m, 3H), 2.37 (s, 3H), 2.65-2.68 (m, 1H), 2.82-2.85 (m, 1H), 2.90 (s, 3H), 3.30 (s, 2H), 3.54-3.63 (m, 1H), 4.09 (dd, 1H, J=8.7, 5.7 Hz), 4.32 (d, 2H, J=6.0 Hz), 4.57 (t, 1H, J=9.0 Hz), 4.67 (br t, 1H, J=5.1 Hz), 4.79 (dd, 1H, J=9.0, 5.7 Hz), 6.55 (d, 1H, J=8.4 Hz), 7.10 (d, 2H, J=8.4 Hz), 7.31-7.40 (m, 7H), 7.45 (d, 1H, J=8.4 Hz); ¹³C NMR (CDCl₃) δ 22.24, 29.62, 30.77, 41.52, 47.10, 53.21, 53.32, 53.47, 59.00, 59.25, 70.93, 108.14, 121.29, 127.17, 127.35, 129.34, 129.60, 129.69, 132.87, 140.83, 141.34, 154.97, 156.95, 158.50, 161.86; ES-MS m/z 573 (M+Na). Anal. Calcd. for C₂₉H₃₄N₄SO₅.0.5CH₂Cl₂: C, 59.74; H, 5.95; N, 9.45. Found: C, 60.04; H, 5.94; N, 9.44.

### EXAMPLE 268

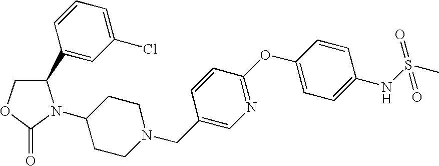

### COMPOUND 268: N-[4-(5-{4-[(R)-4-(3-Chloro-phenyl)-2-oxo-oxazolidin-3-yl]-piperidin-1-ylmethyl}-pyridin-2-yloxy)-phenyl]-methanesulfonamide

A solution of 6-bromo-pyridine-3-carbaldehyde (1.02 g, 5.48 mmol), 4-nitrophenol (761 mg, 5.47 mmol) and K₂CO₃ (529 mg, 3.83 mmol) in DMF (11 mL) was heated to 130° C. for 1 hour. Aqueous work-up and purification afforded 6-(4-nitro-phenoxy)-pyridine-3-carbaldehyde as a yellow solid (1.20 g, 90%).

To the above aldehyde (386 mg, 1.58 mmol) in MeOH (8 mL) was added NaBH₄ (120 mg, 3.17 mmol) and the mixture was stirred at room temperature for 25 minutes. Aqueous work-up afforded the alcohol which was subsequently hydrogenated in MeOH (10 mL) over 10% Pd/C (60 mg) at 45 psi for 2.3 hours. Filtration and purification afforded [6-(4-amino-phenoxy)-pyridin-3-yl]-methanol as a yellow oil (150 mg, 44% over 2 steps).

A mixture of the above alcohol (150 mg, 0.694 mmol) and MnO₂ (85%, 710 mg, 6.94 mmol) in 10% MeOH/CH₂Cl₂ (7 mL) was heated to reflux for 4 hours. The mixture was filtered to give 6-(4-amino-phenoxy)-pyridine-3-carbaldehyde (155 mg).

Following general procedure A, (R)-4-Phenyl-3-piperidin-4-yl-oxazolidin-2-one (195 mg, 0.695 mmol) and the aldehyde (155 mg) afforded (R)-3-{1-[6-(4-amino-phenoxy)-pyridin-3-ylmethyl]-piperidin-4-yl}-4-(3-chloro-phenyl)-oxazolidin-2-one (47 mg, 14% over 2 steps).

To a solution of the above amine (47 mg, 0.098 mmol) and Et₃N (0.014 mL, 0.10 mmol) in CH₂Cl₂ (2.0 mL) at −25° C. was added MsCl (0.0061 mL, 0.079 mmol) in CH₂Cl₂ (2 mL) over 1 hour. The mixture was warmed to room temperature over 2 hours and stirred for an additional 1 hour. A second aliquot of MsCl (0.012 mL, 0.16 mmol) in CH₂Cl₂ (2.0 mL) was added at −25° C. over 1 hour. The mixture was warmed to room temperature over 2 hours and stirred for an additional 1 hour. Standard work-up and purification afforded COMPOUND 268 as a yellow foam (36 mg, 82%). ¹H NMR (CDCl₃) δ 1.27 (ddd, 1H, J=24.6, 12.0, 3.9 Hz), 1.50-1.54 (m, 1H), 1.71-1.75 (m, 1H), 1.83-2.04 (m, 3H), 2.70-2.74 (m, 1H), 2.85-2.89 (m, 1H), 3.02 (s, 3H), 3.37 (s, 2H), 3.57-3.66 (m, 1H), 4.05 (dd, 1H, J=8.7, 5.4 Hz), 4.57 (t, 1H, J=8.7 Hz), 4.76 (dd, 1H, J=9.0, 5.4 Hz), 6.46 (br s, 1H), 6.87 (d, 1H, J=8.4 Hz), 7.11-7.15 (m, 2H), 7.20-7.27 (m, 3H), 7.32-7.35 (m, 3H), 7.61 (dd, 1H, J=8.4, 2.4 Hz), 7.99 (d, 1H, J=2.1 Hz); ¹³C NMR (CDCl₃) δ 29.60, 30.96, 39.64, 52.92, 53.07, 53.44, 58.28, 59.48, 70.74, 111.65, 122.55, 123.39, 125.17, 127.19, 128.99, 129.57, 131.03, 133.58, 135.51, 141.02, 143.13, 148.02, 152.17, 158.44, 163.14; ES-MS m/z 557 (M+1). Anal. Calcd. for C₂₇H₂₉N₄ClSO₅.0.3CH₂Cl₂: C, 56.29; H, 5.12; N, 9.62. Found: C, 56.45; H, 5.16; N, 9.59.

### EXAMPLE 269

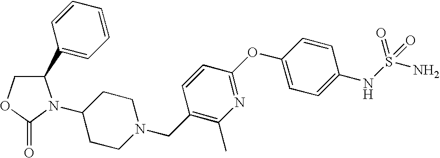

### COMPOUND 269: N-(2-Methyl-4-{6-methyl-5-[4-((R)-2-oxo-4-phenyl-oxazolidin-3-yl)-piperidin-1-ylmethyl]-pyridin-2-yloxy}-phenyl)-sulfamide

Formic acid (0.540 mL, 14.3 mmol) was added dropwise to a solution of chlorosulfonyl isocyanate (1.25 mL, 14.4 mmol) in CH₂Cl₂ (8.0 mL) and the mixture was heated to reflux for 5 hours (Glaxo Group Ltd, Patent, W02004/6923A1). The mixture was concentrated under reduced pressure to afford the desired compound as yellow crystals.

To a solution of (R)-3-{1-[6-(4-amino-phenoxy)-2-methyl-pyridin-3-ylmethyl]-piperidin-4-yl}-4-phenyl-oxazolidin-2-one (see EXAMPLE 256) (203 mg, 0.443 mmol) and Et₃N (0.062 mL, 0.44 mmol) in CH₂Cl₂ (8.9 mL) at −25° C. was added the above sulfonylchloride (51 mg, 0.44 mmol) in CH₂Cl₂ (6 mL) over 40 minutes. The mixture was allowed to warm to room temperature overnight. The mixture was concentrated under reduced pressure and redissolved in CH₂Cl₂ (8 mL). A second aliquot of the above sulfonylchloride (52 mg, 0.45 mmol) in CH₂Cl₂ (6 mL) was added over 45 minutes. The mixture was allowed to warm to room temperature over 1.5 hours. Aqueous work-up and purification afforded COMPOUND 269 as a colourless foam (122 mg, 51%). ¹H NMR(CDCl₃) δ 1.31 (ddd, 1H, J=24.3, 12.0, 3.9 Hz), 1.48-1.52 (m, 1H), 1.68-1.70 (m, 1H), 1.82-2.03 (m, 3H), 2.37 (s, 3H), 2.66-2.70 (m, 1H), 2.82-2.84 (m, 1H), 3.30 (s, 2H), 3.49-3.56 (m, 1H), 4.09 (dd, 1H, J=8.7, 6.0 Hz), 4.58 (t, 1H, J=9.0 Hz), 4.80 (dd, 1H, J=8.7, 5.7 Hz), 5.02 (br s, 2H), 6.52 (d, 1H, J=8.1 Hz), 6.80 (br s, 1H), 7.05 (d, 2H, J=8.7 Hz), 7.23-7.26 (m, 2H), 7.31-7.46 (m, 6H); ¹³C NMR (CDCl₃) δ 21.69, 29.04, 29.96, 52.78, 52.95, 58.70, 58.87, 70.59, 107.60, 121.50, 123.31, 126.73, 126.82, 128.99, 129.23, 133.60, 140.13, 141.24, 151.86, 156.50, 158.19, 161.69; ES-MS m/z 538 (M+1). Anal. Calcd. for C₂₇H₃₁N₅SO₅.0.6CH₂Cl₂: C, 56.32; H, 5.51; N 11.90. Found: C, 56.25; H, 5.49; N, 11.89.

### EXAMPLE 270

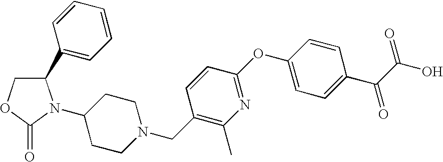

### COMPOUND 270: (4-{6-Methyl-5-[4-((R)-2-oxo-4-phenyl-oxazolidin-3-yl)-piperidin-1-ylmethyl]-pyridin-2-yloxy}-phenyl)-oxo-acetic acid

A solution of DL-4-hydroxymandelic acid (4.20 g, 25.0 mmol) and H₂SO₄ (0.67 mL, 13 mmol) in MeOH (50 mL) was stirred at room temperature for 20 hours. Standard aqueous work-up afforded hydroxy-(4-hydroxy-phenyl)-acetic acid methyl ester as a pink solid (1.98 g, 44%).

A solution of the above phenol (820 mg, 4.50 mmol), 6-chloro-2-methyl-pyridine-3-carbaldehyde (700 mg, 4.50 mmol) and K₂CO₃ (373 mg, 2.70 mmol) in DMF (9.0 mL) was heated to 130° C. for 45 minutes. Standard work-up and purification afforded [4-(5-formyl-6-methyl-pyridin-2-yloxy)-phenyl]-hydroxy-acetic acid methyl ester (198 mg).

Following general procedure A, (R)-4-phenyl-3-piperidin-4-yl-oxazolidin-2-one (162 mg, 0.658 mmol) and the above aldehyde (198 mg) afforded hydroxy-(4-{6-methyl-5-[4-((R)-2-oxo-4-phenyl-oxazolidin-3-yl)-piperidin-1-ylmethyl]-pyridin-2-yloxy}-phenyl)-acetic acid methyl ester (163 mg, 43%) following work-up and purification.

A mixture of the above alcohol (163 mg, 0.307 mmol) and MnO₂ (85%, 314 mg, 3.07 mmol) in CH₂Cl₂ (3 mL) was stirred at room temperature for 3 days. The mixture was filtered through Celite® and concentrated under reduced pressure to afford (4-{6-methyl-5-[4-((R)-2-oxo-4-phenyl-oxazolidin-3-yl)-piperidin-1-ylmethyl]-pyridin-2-yloxy}-phenyl)-oxo-acetic acid methyl ester (124 mg, 77%). Following general procedure H, the above methyl ester (124 mg, 0.234 mmol) afforded COMPOUND 270 as a yellow solid (80 mg, 66%). ¹H NMR (CD₃OD) δ 1.87-1.95 (m, 3H), 2.38-2.48 (m, 4H), 3.03-3.11 (m, 2H), 3.41-3.56 (m, 2H), 3.62-3.72 (m, 1H), 4.18 (dd, 1H, J=8.7, 6.3 Hz), 4.27 (s, 2H), 4.72 (t, 1H, J=8.7 Hz), 5.04 (dd, 1H, J=9.0, 6.3 Hz), 6.92 (d, 1H, J=8.4 Hz), 7.24 (d, 2H, J=8.7 Hz), 7.40-7.50 (m, 5H), 7.86 (d, 1H, J=8.4 Hz), 8.05 (d, 2H, J=8.7 Hz); ES-MS m/z 516 (M+1). Anal. Calcd. for C₂₉H₂₉N₃O₆.0.4CH₂Cl₂.0.5CH₄O: C, 63.50; H, 5.67; N, 7.43. Found: C, 63.33; H, 5.68; N, 7.43.

### EXAMPLE 271

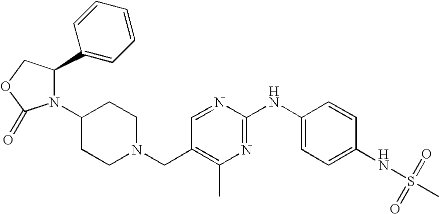

### COMPOUND 271: N-(4-{4-Methyl-5-[4-((R)-2-oxo-4-phenyl-oxazolidin-3-yl)-piperidin-1-ylmethyl]-pyrimidin-2-ylamino}-phenyl)-methanesulfonamide

A mixture of 4-bromoaniline (5.16 g, 30.0 mmol), di-tert-butyl dicarbonate (8.72 g, 40.0 mmol), and DIPEA (5.81 g, 45.0 mmol) in DMF (40 mL) was stirred at room temperature for 24 h. After concentration saturated aqueous NaHCO₃ (100 ml) was added and extraction with CH₂Cl₂ (3×100 mL) was performed. The combined extract was dried over anhydrous Na₂SO₄. After filtration the solvent was removed, and the residue was purified by flash chromatography on silica gel (CH₂Cl₂) to afford (4-bromo-phenyl)-carbamic acid tert-butyl ester as a white solid (7.60 g, 93%). ¹H NMR (CDCl₃) δ 1.52 (s, 9H), 6.47 (br s, 1H), 7.24-7.28 (m, 2H), 7.37-7.40 (m, 2H).

Under N₂, to a dry flask charged with 2-amino-4-methyl-pyrimidine-5-carboxylic acid ethyl ester (2.50 g, 13.8 mmol), (4-bromo-phenyl)-carbamic acid tert-butyl ester (4.08 g, 15.0 mmol), tert-BuOK (1.90 g, 17.0 mmol), 4,5-bis(diphenylphosphino)-9,9-dimethylxanthene (0.176 g, 0.304 mmol) and Pd₂(dba)₃ (0.126 g, 0.138 mmol) was added anhydrous toluene (100 mL). The mixture was degassed and filled with N₂ twice, and then was stirred at 100° C. for 48 h. After the mixture was cooled to room temperature, saturated aqueous NH₄Cl solution (30 mL) and brine (30 mL) were added and the mixture was extracted with EtOAc (3×50 mL). The combined extract was dried over anhydrous Na₂SO₄. After filtration the solvent was removed, and the residue was purified by flash chromatography on silica gel (CH₂Cl₂, then EtOAc/hexanes from 1:4 to 1:1 in v/v) followed by recrystallization from EtOAc/hexanes, affording 2-(4-tert-butoxycarbonylamino-phenylamino)-4-methyl-pyrimidine-5-carboxylic acid ethyl ester as a pale yellow solid (3.05 g, 60%). ¹H NMR (CDCl₃) δ 1.38 (t, 3H, J=7.2 Hz), 1.52 (s, 9H), 2.72 (s, 3H), 4.34 (q, 2H, J=7.2 Hz), 6.47 (br s, 1H), 7.31-7.36 (m, 3H), 7.54-7.58 (m, 2H), 8.90 (s, 1H).

Under N₂, to a solution of 2-(4-tert-butoxycarbonylamino-phenylamino)-4-methyl-pyrimidine-5-carboxylic acid ethyl ester (2.50 g, 6.72 mmol) in anhydrous THF (80 mL) cooled at −10° C. was added DIBAL-H (1.0 M, toluene, 47 mL, 47 mmol). After addition the cooling bath was removed and the mixture was stirred at room temperature for 6 h. Saturated aqueous NH₄Cl (40 mL) were added and the mixture was extracted with EtOAc (3×50 mL). The combined extract was dried over anhydrous Na₂SO₄. After filtration the solvent was removed to afford the crude product (1.90 g)

The crude product was dissolved in CH₂Cl₂ (150 mL). MnO₂ (6.0 g, 70 mmol) was added, and the suspension was stirred at room temperature for 5 h. The suspension was then filtered through a Celite® cake. The filtrate was collected and concentrated, and the residue was purified by flash chromatography on silica gel (EtOAc/hexanes, 1:1 in v/v) to afford [4-(5-formyl-4-methyl-pyrimidin-2-ylamino)-phenyl]-carbamic acid tert-butyl ester as a pale yellow solid (1.83 g, 83% two steps). ¹H NMR (CDCl₃) δ 1.52 (s, 9H), 2.72 (s, 3H), 6.50 (br s, 1H), 7.37 (d, 2H, J=8.7 Hz), 7.49 (br s, 1H), 7.57 (d, 2H, J=8.7 Hz), 8.69 (s, 1H), 9.99 (s, 1H).

Following General Procedure A: using (R)-4-phenyl-3-piperidin-4-yl-1-(tetrahydro-pyran-4-yl)-imidazolidin-2-one (0.630 g, 1.92 mmol), [4-(5-formyl-4-methyl-pyrimidin-2-ylamino)-phenyl]-carbamic acid tert-butyl ester (0.368 g, 1.50 mmol), and 3 drop AcOH. Purification of the crude product by flash chromatography on silica gel (EtOAc) gave the ester as a pale yellow solid. The solid was treated with TFA/CH₂Cl₂ following General Procedure C to afford (R)-3-{1-[2-(4-amino-phenylamino)-4-methyl-pyrimidin-5-ylmethyl]-piperidin-4-yl}-4-phenyl-oxazolidin-2-one as a pale yellow solid (0.273 g, 40% two steps).

To a solution of the above amine (0.100 g, 0.219 mmol) and pyridine (0.098 g, 1.2 mmol) in CH₂Cl₂ (3 mL) was added methanesulfonyl chloride (0.050 g, 0.44 mmol). After the mixture was stirred at room temperature for 2 h water (10 mL) was added. Extraction with CH₂Cl₂ (4×10 mL) was performed, and the combined extract was dried over anhydrous Na₂SO₄. After filtration the solvent was removed, and the residue was purified by flash chromatography on silica gel (CH₂Cl₂/MeOH, 20:1 in v/v) to afford COMPOUND 271 as a white solid (0.026 g, 22%). ¹H NMR (CDCl₃) δ 1.23-1.27 (m, 1H), 1.48-1.52 (m, 1H), 1.69-1.73 (m, 1H), 1.89-1.99 (m, 3H), 2.38 (s, 3H), 2.66-2.69 (m, 1H), 2.82-2.85 (m, 1H), 2.97 (s, 3H), 3.26 (s, 2H), 3.54-3.58 (m, 1H), 4.10 (dd, 1H, J=8.7,5.7 Hz), 4.57 (t, 1H, J=8.7 Hz), 4.78 (dd, 1H, J=8.7, 5.7 Hz), 6.63 (br s, 1H), 7.20-7.25 (m, 3H), 7.30-7.41 (m, 5H), 7.60-7.65 (m, 2H), 8.07 (s, 1H); ES-MS m/z 537 (M+H). Anal. Calcd. for C₂₇H₃₂N₆O₄.0.9CH₂Cl₂: C, 54.66; H, 5.56, N, 13.71. Found: C, 55.01; H, 5.47; N, 13.36.

### EXAMPLE 272

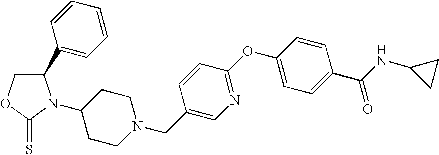

### COMPOUND 272: N-Cyclopropyl-4-{5-[4-((R)-4-phenyl-2-thioxo-oxazolidin-3-yl)-piperidin-1-ylmethyl]-pyridin-2-yloxy}-benzamide

Following general procedure A, (R)-4-phenyl-3-piperidin-4-yl-oxazolidine-2-thione (100 mg, 0.381 mmol) and N-cyclopropyl-4-(5-formyl-pyridin-2-yloxy)-benzamide (107 mg, 0.379 mmol) afforded COMPOUND 272 as a white solid (100 mg, 50%). ¹H NMR (CDCl₃) δ 0.82-1.02 (m, 5H), 1.55-1.60 (m, 1H), 1.79-2.10 (m, 4H), 2.64-2.68 (m, 1H), 2.88-2.91 (m, 2H), 3.38 (s, 2H), 4.30-4.36 (m, 2H), 4.72 (t, 1H, J=9.0 Hz), 4.94 (dd, 1H, J=9.3, 3.9 Hz), 6.32 (br s, 1H), 6.86 (d, 1H, J=8.4 Hz), 7.12 (d, 2H, J=8.4 Hz), 7.24-7.26 (m, 1H), 7.37-7.38 (m, 3H), 7.58 (dd, 1H, J=8.1, 1.8 Hz), 7.76 (d, 2H, J=8.4 Hz), 7.99 (s, 1H); ¹³C NMR (CDCl₃) δ 7.19, 23.53, 29.45, 30.50, 52.84, 52.94, 57.32, 59.43, 61.65, 75.30, 111.99, 121.11, 126.91, 128.99, 129.21, 129.65, 129.79, 130.88, 140.15, 140.98, 148.21, 157.37, 162.71, 168.59, 187.78; ES-MS m/z 529 (M+1).

### EXAMPLE 273

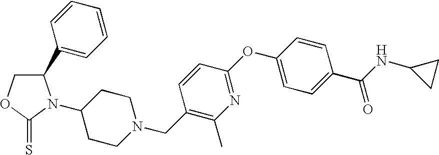

### COMPOUND 273: N-Cyclopropyl-4-{6-methyl-5-[4-((R)-4-phenyl-2-thioxo-oxazolidin-3-yl)-piperidin-1-ylmethyl]-pyridin-2-yloxy}-benzamide

Following general procedure A, (R)-4-phenyl-3-piperidin-4-yl-oxazolidine-2-thione (74 mg, 0.28 mmol) and N-cyclopropyl-4-(5-formyl-6-methyl-pyridin-2-yloxy)-benzamide (83 mg, 0.28 mmol) afforded COMPOUND 273 as a white solid (73 mg, 48%). ¹H NMR (CDCl₃) δ 0.81-0.96 (m, 3H), 1.50-1.59 (m, 2H), 1.76-1.99 (m, 4H), 2.07-2.15 (m, 1H), 2.35 (s, 3H), 2.60-2.64 (m, 1H), 2.85-2.89 (m, 2H), 3.32 (s, 2H), 4.31-4.41 (m, 2H), 4.72 (t, 1H, J=9.0 Hz), 0.94 (dd, 1H, J=9.3, 3.9 Hz), 6.36 (br s, 1H), 6.58 (d, 1H, J=8.1 Hz), 7.09 (d, 2H, J=8.7 Hz), 7.236-7.26 (m, 2H), 7.36-7.41 (m, 3H), 7.44 (d, 1H, J=8.1 Hz), 7.74 (d, 2H, J=8.4 Hz); ¹³C NMR (CDCl₃) δ 7.16, 22.21, 23.53, 29.58, 30.58, 53.04, 53.14, 57.42, 59.26, 61.66, 75.26, 108.77, 120.40, 126.95, 127.71, 128.95, 129.65, 129.76, 130.37, 140.14, 141.44, 157.11, 157.97, 161.30, 168.66, 187.73; ES-MS m/z 565 (M+Na). Anal. Calcd. for C₃₁H₃₄N₄O₃S.0.23CH₄O.0.61CH₂Cl₂: C, 63.52; H, 6.05; N, 9.31. Found: C, 63.54; H, 6.00; N, 9.21.

### EXAMPLE 274

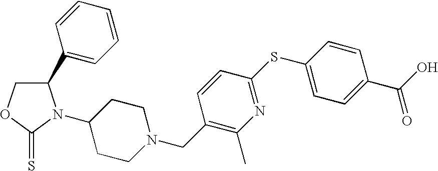

### COMPOUND 274: 4-{6-Methyl-5-[4-((R)-4-phenyl-2-thioxo-oxazolidin-3-yl)-piperidin-1-ylmethyl]-pyridin-2-ylsulfanyl}-benzoic acid

Following general procedure A, (R)-4-phenyl-3-piperidin-4-yl-oxazolidine-2-thione (66.5 mg, 0.253 mmol) and 4-(5-formyl-6-methyl-pyridin-2-ylsulfanyl)-benzoic acid methyl ester (72.5 mg, 0.253 mmol) afforded the methyl ester. Following general procedure H, the methyl ester afforded COMPOUND 274 as a white solid (70 mg, 53% over 2 steps). ¹H NMR (CD₃OD) δ 1.14-1.20 (m, 1H), 1.76-2.06 (m, 3H), 2.42 (s, 3H), 2.69-2.85 (m, 2H), 3.06-3.09 (m, 1H), 3.22-3.29 (m, 1H), 3.93-3.96 (m, 2H), 4.23-4.27 (m, 1H), 4.31-4.44 (m, 1H), 4.66-4.72 (m, 1H), 5.07-5.11 (m, 1H), 6.81-6.84 (m, 1H), 7.21-7.32 (m, 5H), 7.50-7.52 (m, 3H), 7.94-7.96 (m, 2H); ¹³C NMR (CD₃OD) δ 14.87, 21.27, 22.54, 28.39, 29.31, 53.52, 53.64, 56.32, 58.58, 61.95, 63.10, 77.04, 79.89, 121.89, 128.38, 130.74, 130.96, 132.22, 134.95, 138.65, 141.67, 142.24, 160.79, 173.30, 189.57; ES-MS m/z 520 (M+1). Anal. Calcd. for C₂₈H₂₉N₃O₃S₂.0.77CH₄O0.61CH₂Cl₂: C, 59.21; H, 5.63; N, 7.05. Found: C, 59.19; H, 5.68; N, 7.17.

### EXAMPLE 275

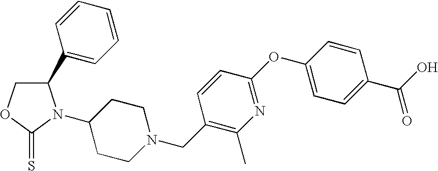

### COMPOUND 275: 4-{6-Methyl-5-[4-((R)-4-phenyl-2-thioxo-oxazolidin-3-yl)-piperidin-1-ylmethyl]-pyridin-2-yloxy}-benzoic acid

Following general procedure A, (R)-4-phenyl-3-piperidin-4-yl-oxazolidine-2-thione (78 mg, 0.30 mmol) and 4-(5-formyl-6-methyl-pyridin-2-yloxy)-benzoic acid methyl ester (81 mg, 0.30 mmol) afforded the methyl ester. Following general procedure H, the methyl ester afforded COMPOUND 275 as a white solid (85 mg, 57% over 2 steps). ¹H NMR (CD₃OD) δ 1.28-1.41 (m, 1H), 1.77-1.81 (m, 1H), 2.02-2.17 (m, 2H), 2.35 (s, 3H), 2.76-2.97 (m, 2H), 3.13-3.17 (m, 1H), 3.35-3.39 (m, 1H), 4.04 (s, 2H), 4.25 (dd, 1H, J=9.3, 3.9 Hz), 4.41-4.49 (m, 1H), 4.70 (t, 1H, J=9.0 Hz), 5.11 (dd, 1H, J=9.3, 3.9 Hz), 6.75 (d, 1H, J=8.1 Hz), 7.04-7.08 (m, 2H), 7.21-7.32 (m, 5H), 7.75 (d, 1H, J=8.1 Hz), 7.92-7.96 (m, 2H); ¹³C NMR (CD₃OD δ 22.53, 28.19, 29.09, 53.38, 56.10, 58.20, 63.07, 77.09, 79.89, 111.16, 121.76, 122.33, 128.37, 128.82, 130.74, 130.98, 133.09, 141.66, 145.50, 159.71, 159.94, 164.23, 169.72, 189.58; ES-MS m/z 504 (M+1). Anal. Calcd. for C₂₈H₂₉N₃O₄S.0.73CH₄O.0.65CH₂Cl₂: C, 60.58; H, 5.75; N, 7.21. Found: C, 60.58; H, 5.76; N, 7.24.

### EXAMPLE 276

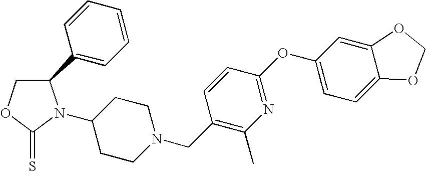

### COMPOUND 276: (R)-3-{1-[6-(Benzo[1,3]dioxol-5-yloxy)-2-methyl-pyridin-3-ylmethyl]-piperidin-4-yl}-4-phenyl-oxazolidine-2-thione

A solution of 6-bromo-2-methyl-pyridine-3-carbaldehyde (638 mg, 3.19 mmol), 3,4-(methylenedioxy)phenol (431 mg, 3.19 mmol) and K₂CO₃ (431 mg, 3.19 mmol) in DMF (20 mL) was heated to 115° C. for 2 hours. Standard work-up and purification afforded 6-(benzo[1,3]dioxol-5-yloxy)-2-methyl-pyridine-3-carbaldehyde (460 mg, 56%).

Following general procedure A, (R)-4-phenyl-3-piperidin-4-yl-oxazolidine-2-thione (50 mg, 0.19 mmol) and the above aldehyde (49 mg, 0.19 mmol) afforded COMPOUND 276 as a white solid (45 mg, 46%). ¹H NMR (CDCl₃) δ 0.86-0.99 (m, 1H), 1.55-1.59 (m, 1H), 1.75-1.98 (m, 3H), 2.06-2.14 (m, 1H), 2.38 (s, 3H), 2.61-2.64 (m, 1H), 2.85-2.88 (m, 1H), 3.31 (s, 2H), 4.32-4.21 (m, 2H), 4.73 (t, 1H, J=9.0 Hz), 4.94 (dd, 1H, J=9.0, 3.9 Hz), 5.98 (s, 2H), 6.47 (d, 1H, J=8.4 Hz), 6.56 (dd, 1H, J=8.4, 1.8 Hz), 6.64 (d, 1H, J=1.8 Hz), 6.77 (d, 1H, J=6.0 Hz), 7.25-7.27 (m, 2H), 7.36-7.38 (m, 4H); ¹³C NMR (CDCl₃) δ 22.30, 29.59, 30.61, 52.98, 53.10, 57.46, 59.32, 61.66, 75.24, 101.95, 103.78, 107.03, 108.60, 113.75, 126.61, 126.96, 129.66, 129.76, 140.16, 141.28, 144.75, 148.57, 149.32, 157.08, 162.81, 187.74; ES-MS m/z 504 (M+1). Anal. Calcd. for C₂₈H₃₉N₃O₄S.0.23CH₄O: C, 66.36; H, 5.90; N, 8.23. Found: C, 66.51; H, 5.83; N, 8.06.

Examples 277 to 297 were prepared following the scheme illustrated below. RCHO is as defined in the table and Y is as defined in the individual examples.

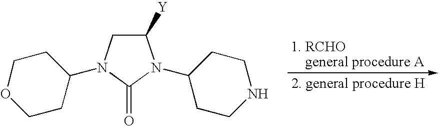

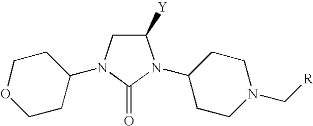

| Exam- | |
|---|---|
| ple | RCHO |
| 277 | 4-(6-chloro-5-formyl-pyridin-2-yloxy)-benzoic acid methyl ester |
| 278 | 4-(5-formyl-6-methyl-pyridin-2-yloxy)-benzoic acid methyl ester |
| 279 | 4-(5-formyl-6-methyl-pyridin-2-ylsulfanyl)-benzoic acid methyl |
| | ester |
| 280 | 4-(6-fluoro-5-formyl-pyridin-2-yloxy)-benzoic acid methyl ester |
| 281 | 4-(6-chloro-5-formyl-pyridin-2-yloxy)-benzoic acid methyl ester |
| 282 | 4-(5-formyl-pyrimidin-2-ylsulfanyl)-benzoic acid methyl ester |
| 283 | 4-(5-formyl-6-methyl-pyridin-2-ylsulfanyl)-benzoic acid methyl |
| | ester |
| 284 | 4-(5-formyl-6-methyl-pyridin-2-yloxy)-benzoic acid methyl ester |
| 285 | 4-[(5-formyl-pyrimidin-2-yl)-methyl-amino]-benzoic acid methyl |
| | ester |
| 286 | 4-(5-formyl-4-methyl-pyrimidin-2-yloxy)-benzoic acid methyl |
| | ester (see EXAMPLE 93) |
| 287 | 4-(5-formyl-6-methyl-pyridin-2-yloxy)-benzoic acid methyl ester |
| 288 | 4-(5-formyl-pyridin-2-yloxy)-benzoic acid methyl ester |
| 289 | 4-(5-formyl-6-methyl-pyridin-2-ylsulfanyl)-benzoic acid methyl |
| | ester |
| 290 | 4-(5-formyl-6-methyl-pyridin-2-yloxy)-benzoic acid methyl ester |
| 291 | 4-(5-formyl-6-methyl-pyridin-2-ylsulfanyl)-benzoic acid methyl |
| | ester |
| 292 | 4-(5-formyl-pyridin-2-ylsulfanyl)-benzoic acid methyl ester |
| 293 | 4-(5-formyl-pyrimidin-2-ylsulfanyl)-benzoic acid methyl ester |
| 294 | 4-(5-formyl-6-methyl-pyridin-2-yloxy)-benzoic acid methyl ester |
| 295 | 4-(5-formyl-6-methyl-pyridin-2-ylsulfanyl)-benzoic acid methyl |
| | ester |
| 296 | [4-(5-formyl-6-methyl-pyridin-2-ylsulfanyl)-phenyl]-acetic acid |
| | methyl ester |
| 297 | 4-(5-formyl-4-methyl-pyridin-2-yloxy)-benzoic acid methyl ester |

### EXAMPLE 277

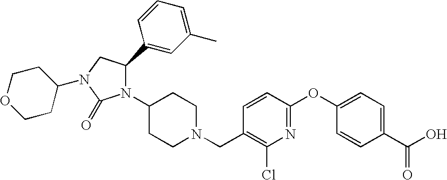

### COMPOUND 277: 4-(6-Chloro-5-{4-[(R)-2-oxo-3-(tetrahydro-pyran-4-yl)-5-m-tolyl-imidazolidin-1-yl]-piperidin-1-ylmethyl}-pyridin-2-yloxy)-benzoic acid

COMPOUND 277 was isolated as a white solid (135 mg, 74% over 2 steps). ¹H NMR (CD₃OD) δ 1.60-1.90 (m, 7H), 2.34 (m, 1H), 2.35 (s, 3H), 2.94 (q, 2H, J=12.0 Hz), 3.14 (t, 1H, J=8.1 Hz), 3.33-3.60 (m, 5H), 3.77 (t, 1H, J=9.3 Hz), 3.95 (m, 3H), 4.23 (s, 2H), 4.70 (t, 1H, J=8.1 Hz), 7.08 (d, 1H, J=8.4 Hz), 7.12-7.33 (m, 6H), 7.99 (d, 1H, J=8.1 Hz), 8.09 (dd, 2H, J=8.4, 1.2 Hz); ¹³C NMR (CD₃OD) δ 20.61, 26.79, 27.52, 29.80, 30.20, 48.53, 49.44, 50.01, 52.37 (2C), 56.14, 57.09, 67.16, 67.26, 111.24, 120.02, 120.98 (2C), 124.10, 127.74, 128.36, 129.17, 129.35, 131.72 (2C), 139.08, 141.98, 146.13, 150.26, 157.37, 160.62, 163.07, 168.24; ES-MS m/z 605 (M+H). Anal. Calcd. for C₃₃H₃₇ClN₄O₅.1.1CH₂Cl₂: C, 58.63; H, 5.66; N, 8.02. Found: C, 58.67; H, 5.84; N, 8.02.

### EXAMPLE 278

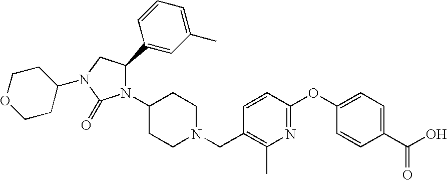

### COMPOUND 278: 4-(6-Methyl-5-{4-[(R)-2-oxo-3-(tetrahydro-pyran-4-yl)-5-m-tolyl-imidazolidin-1-yl]-piperidin-1-ylmethyl}-pyridin-2-yloxy)-benzoic acid

COMPOUND 278 was isolated as a white solid (112 mg, 65% over 2 steps). ¹H NMR (CD₃OD) δ 1.50-1.80 (m, 7H), 2.14 (dq, 1H, J=12.6, 3.6 Hz), 2.34 (s, 3H), 2.42 (s, 3H), 2.45 (q, 1H, J=12.9 Hz), 3.03 (d, 1H, J=11.4 Hz), 3.12 (t, 1H, J=8.1 Hz), 3.15 (m, 1H), 3.48 (t, 2H, J=11.7 Hz), 3.55 (m, 1H), 3.76 (t, 1H, J=9.3 Hz), 3.78 (s, 2H), 3.92 (m, 3H), 4.66 (m, 1H), 6.80 (d, 1H, J=8.4 Hz), 7.13 (d, 2H, J=8.7 Hz), 7.16-7.30 (m, 4H), 7.72 (d, 1H, J=8.4 Hz), 8.03 (d, 2H, J=8.4 Hz); ¹³C NMR (CD₃OD) δ 20.58, 21.10, 27.43, 28.60, 29.82, 30.18, 48.57, 49.44, 50.96, 52.37, 52.54, 56.86, 57.01, 67.17, 67.26, 109.46, 120.07 (3C), 122.53, 124.05, 127.61, 129.07, 129.24, 131.62 (2C), 138.96, 142.27, 143.78, 157.96, 158.20, 160.75, 162.53, 169.55; ES-MS m/z 585 (M+H). Anal. Calcd. for C₃₄H₄₀N₄O₅.0.6CH₂Cl₂: C, 65.38; H, 6.53; N, 8.81. Found: C, 65.19; H, 6.63; N, 8.71.

### EXAMPLE 279

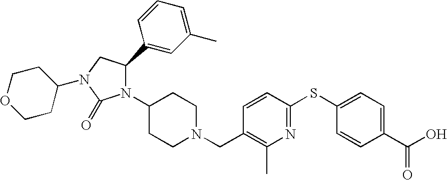

### COMPOUND 279: 4-(6-Methyl-5-{4-[(R)-2-oxo-3-(tetrahydro-pyran-4-yl)-5-m-tolyl-imidazolidin-1-yl]-piperidin-1-ylmethyl}-pyridin-2-ylsulfanyl)-benzoic acid

COMPOUND 279 was isolated as a white solid (106 mg, 60% over 2 steps). ¹H NMR (CD₃OD) δ 1.50-1.80 (m, 7H), 2.15 (dq, 1H, J=12.6, 3.6 Hz), 2.32 (s, 3H), 2.49 (s, 3H), 2.55 (q, 1H, J=12.9 Hz), 3.07 (d, 1H, J=11.4 Hz), 3.12 (t, 1H, J=8.1 Hz), 3.17 (d, 1H, J=12.3 Hz), 3.47 (t, 2H, J=11.7 Hz), 3.55 (m, 1H), 3.75 (t, 1H, J=9.3 Hz), 3.83 (s, 2H), 3.94 (m, 3H), 4.65 (m, 1H), 6.89 (d, 1H, J=8.7 Hz), 7.16 (m, 3H), 7.25 (t, 1H, J=7.5 Hz), 7.56 (m, 3H), 8.03 (d, 2H, J=8.1 Hz); ¹³C NMR (CD₃OD) δ 20.53, 21.29, 27.01, 27.78, 29.79, 30.20, 48.53, 49.45, 50.41, 52.26, 52.45, 56.77, 57.10, 67.16, 67.26, 120.12, 122.60 (2C), 124.07, 127.67, 129.11, 129.31, 130.84 (2C), 133.82 (2C), 136.43, 139.04, 141.06, 142.00, 159.35, 160.67, 160.75, 169.15; ES-MS m/z 601 (M+H). Anal. Calcd. for C₃₄H₄₀N₄O₄S.0.8CH₂Cl₂: C, 62.50; H, 6.27; N, 8.38. Found: C, 62.54; H, 6.36; N, 8.36.

### EXAMPLE 280

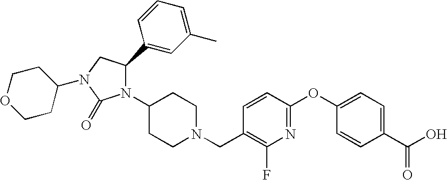

### COMPOUND 280: 4-(6-Fluoro-5-{4-[2-oxo-3-(tetrahydro-pyran-4-yl)-5-m-tolyl-imidazolidin-1-yl]-piperidin-1-ylmethyl}-pyridin-2-yloxy)-benzoic acid

COMPOUND 280 was isolated as a white solid (16 mg, 12% over 2 steps). ¹H NMR (CDCl₃) δ 1.37 (dq, 1H, J=12.0, 3.6 Hz), 1.50 (d, 1H, J=12.0 Hz), 1.66 (m, 4H), 1.77 (d, 1H, J=11.7 Hz), 2.19 (m, 2H), 2.36 (t, 1H, J=11.1 Hz), 3.00 (d, 1H, J=11.4 Hz), 3.05 (m, 1H), 3.27 (d, 1H, J=11.4 Hz), 3.49 (m, 3H), 3.63 (m, 2H), 3.85 (m, 1H), 4.03 (m, 3H), 4.54 (m, 1H), 6.60 (d, 1H, J=8.1 Hz), 6.95 (m, 1H), 7.04 (m, 2H), 7.18 (d, 2H, J=8.7 Hz), 7.70 (t, 1H, J=8.7 Hz), 7.97 (d, 2H, J=8.7 Hz); ES-MS m/z 589 (M+H).

### EXAMPLE 281

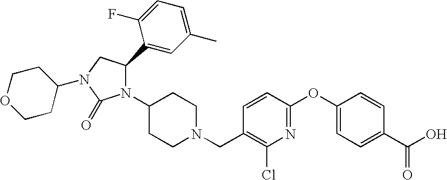

### COMPOUND 281: 4-(6-Chloro-5-{4-[(R)-5-(2-fluoro-5-methyl-phenyl)-2-oxo-3-(tetrahydro-pyran-4-yl)-imidazolidin-1-yl]-piperidin-1-ylmethyl}-pyridin-2-yloxy)-benzoic acid

COMPOUND 281 was isolated as a pale yellow powder (76.0 mg, 48% over 2 steps). ¹H NMR (CDCl₃) δ 1.40-1.82 (m, 6H), 2.00-2.50 (m, 3H), 2.21 (s, 3H), 2.99-3.18 (m, H), 3.20-3.32 (m, 1H), 3.48 (m, 2H), 3.65-3.81 (m, 4H), 3.98-4.06 (m, 3H), 4.93 (dd, 1H, J=9, 6 Hz), 6.69 (d, 1H, J=8.1 Hz), 6.77 (t, 1H, J=8.4 Hz), 6.90-6.96 (m, 1H), 7.13 (m, 1H), 7.14 (d, 2H, J=8.1 Hz), 7.80 (d, 1H, J=6.3 Hz), 8.02 (d, 2H, J=8.1 Hz); ¹³C NMR (CDCl₃) δ 21.1, 28.2, 29.9, 30.3, 30.5, 47.7, 49.1, 51.4, 52.8, 52.9, 53.9, 57.1, 67.5, 67.6, 110.7, 115.9 (d, J=21.5 Hz), 120.6, 124.7, 128.7, 128.9, 129.4, 130.7 (d, J=7.7 Hz), 132.1, 134.5, 144.5, 149.3, 157.3, 158.6 (d, J=245 Hz), 160.2, 161.9, 169.8; ES-MS m/z 623 (M+H). Anal. Calcd. for C₃₃H₃₆N₄O₅ClF.1.2CH₂Cl₂: C, 56.66; H, 5.34; N, 7.73. Found: C, 56.92; H, 5.29; N, 7.71.

### EXAMPLE 282

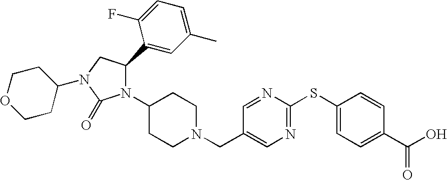

### COMPOUND 282: 4-(5-{4-[(R)-5-(2-Fluoro-5-methyl-phenyl)-2-oxo-3-(tetrahydro-pyran-4-yl)-imidazolidin-1-yl]-piperidin-1-ylmethyl}-pyrimidin-2-ylsulfanyl)-benzoic acid

COMPOUND 282 was isolated as a yellow solid (59.3 mg, 64% over 2 steps). ¹H NMR (CDCl₃) δ 1.25-1.38 (m, 1H), 1.50-1.80 (m, 6H), 2.00-2.29 (m, 3H), 2.17 (s, 3H), 3.00 (d, 1H, J=9 Hz), 3.10 (t, 1H, J=7.5 Hz), 3.24 (d, 1H, J=9 Hz), 3.46-3.60 (m, 4H), 3.66 (br t, 1H), 3.88 (m, 1H), 3.99-4.05 (m, 3H), 4.91 (t, 1H, J=7.5 Hz), 6.67 (t, 1H, J=9 Hz), 6.87 (m, 1H), 7.09 (d, 1H, J=5.7 Hz), 7.65 (d, 2H, J=8.1 Hz), 7.98 (d, 2H, J=8.1 Hz), 8.37 (s, 1H); ¹³C NMR (CDCl₃) δ 21.1, 27.8, 29.8, 30.2, 30.5, 47.6, 49.1, 49.2, 51.2, 52.6, 52.7, 56.3, 67.5, 67.6, 115.9 (d, J=21.8 Hz), 124.6, 128.7, 128.8, 130.7 (d, J=7.5 Hz), 131.0, 132.9, 134.4, 134.5, 135.1, 158.5 (d, J=242 Hz), 159.4, 160.2, 169.1, 172.3; ES-MS m/z 606 (M+H). Anal. Calcd. for C₃₂H₃₆N₅O₄FS.1.7CH₃OH: C, 61.31; H, 6.53; N, 10.61. Found: C, 61.30; H, 6.16; N, 10.35.

### EXAMPLE 283

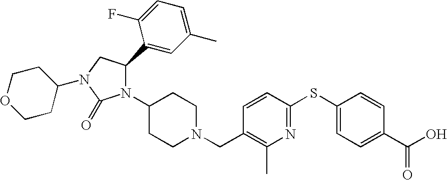

### COMPOUND 283: 4-(5-{4-[(R)-5-(2-Fluoro-5-methyl-phenyl)-2-oxo-3-(tetrahydro-pyran-4-yl)-imidazolidin-1-yl]-piperidin-1-ylmethyl}-6-methyl-pyridin-2-ylsulfanyl)-benzoic acid

COMPOUND 283 was isolated as a yellow powder (30.5 mg, 35% over 2 steps). ¹H NMR (CD₃OD) δ 1.30-1.50 (m, 1H), 1.58-1.79 (m, 6H), 1.98-2.20 (m, 1H), 2.29 (s, 3H), 2.30-2.38 (m, 2H), 2.48 (s, 3H), 2.93 (d, 1H, J=11.1 Hz), 3.06 (d, 1H, J=10.2 Hz), 3.20 (dd, 1H, J=8.7, 6.6 Hz), 3.47 (t, 2H, J=11.7 Hz), 3.59 (m, 1H), 3.66 (s, 2H), 3.79 (t, 1H, J=9.0 Hz), 3.90-3.99 (m, 3H), 4.99 (dd, 1H, J=9.9, 6.6 Hz), 6.87 (d, 1H, J=8.1 Hz), 6.98 (dd, 1H, J=10.5, 8.4 Hz), 7.10-7.17 (m, 1H), 7.20 (d, 1H, J=6.9 Hz), 7.51 (d, 1H, J=8.4 Hz), 7.55 (d, 2H, J=8.1 Hz), 8.01 (d, 2H, J=8.1 Hz); ¹³C NMR (CD₃OD) δ 21.2, 22.4, 29.2, 30.5, 31.2, 31.6, 50.9, 52.0, 52.7, 53.9, 54.2, 55.3, 58.9, 68.6, 68.7, 117.2 (d, J=21.4 Hz), 121.6, 127.0, 129.8, 130.0, 130.5, 132.0 (d, J=7.5 Hz), 132.3, 134.9, 136.1, 137.6, 141.8, 160.3, 160.5 (d, J=243 Hz), 160.9, 161.9; ES-MS m/z 619 (M+H). Anal. Calcd. for C₃₄H₃₉N₄O₄FS.0.4CH₂Cl₂: C, 63.30; H, 6.15; N, 8.58. Found: C, 63.32; H, 6.28; N, 8.39.

### EXAMPLE 284

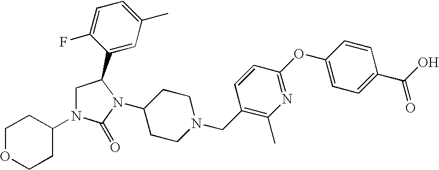

### COMPOUND 284: 4-(5-{4-[(R)-5-(2-Fluoro-5-methyl-phenyl)-2-oxo-3-(tetrahydro-pyran-4-yl)-imidazolidin-1-yl]-piperidin-1-ylmethyl}-6-methyl-pyridin-2-yloxy)-benzoic acid

COMPOUND 284 was isolated as a white solid (922 mg, 74% over 2 steps). ¹H NMR (CDCl₃) δ 1.25-1.42 (m, 1H), 1.52 (br d, 1H, J=12.0 Hz), 1.64-1.74 (m, 5H), 1.95-2.27 (m, 6H), 2.21 (s, 3H), 2.90 (br d, 1H, J=10.5 Hz), 3.06-3.10 (m, 2H), 3.44-3.55 (m, 5H), 3.67 (t, 1H, J=9.1 Hz), 3.70-3.82 (m, 1H), 3.97-4.10 (m, 3H), 4.29 (dd, 1H, J=9.3, 6.3 Hz), 6.57 (d, 11H, J=8.4 Hz), 6.66 (br s, 1H), 6.75-6.81 (m, 1H), 6.92-6.97 (m, 1H), 7.07 (d, 2H, J=8.4 Hz), 7.10-7.23 (m, 1H), 7.55 (d, 1H, J=8.1 Hz), 7.96 (d, 2H, J=8.7 Hz); ¹³C NMR (DMSO) δ 21.1, 22.4, 29.4, 30.1, 30.5, 47.4, 49.4, 50.0, 52.5, 53.4, 58.8, 67.2, 67.3, 109.9, 116.2, 120.6, 127.2, 128.4, 129.8, 131.0, 132.0, 134.7, 142.4, 156.9, 159.2, 159.8, 160.9, 167.7; ES-MS m/z 603 (M+H). Anal. Calcd. for C₃₄H₃₈N₄FO₅.1.7H₂O.0.3CH₂Cl₂: C, 60.96; H, 6.86; N, 10.36. Found: C, 60.70; H, 10.36; N, 10.75.

### EXAMPLE 285

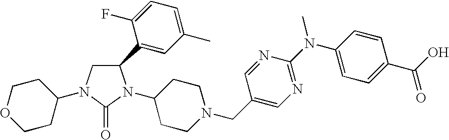

### COMPOUND 285: 4-[(5-{4-[(R)-5-(2-Fluoro-5-methyl-phenyl)-2-oxo-3-(tetrahydro-pyran-4-yl)-imidazolidin-1-yl]-piperidin-1-ylmethyl}-pyrimidin-2-yl)-methyl-amino]-benzoic acid

To a solution of 5-bromo-2-chloropyrimidine (974 mg, 5.03 mmol) and methyl 4-aminobenzoate (764 mg, 5.03 mmol) in THF (25 mL) cooled to −20° C. was added ^{t}BuOK (1.50 g, 12.7 mmol) and the mixture was stirred at −20° C. for 6 hours. The reaction was quenched with NH₄.H₂O and conc. HCl and the resulting precipitate was collected by filtration and dried in vacuo to afford 4-(5-bromo-pyrimidin-2-ylamino)-benzoic acid methyl ester (1.11 g). A mixture of the bromide, tributyl(vinyl)tin (1.3 mL, 3.9 mmol) and bis(triphenylphosphine)palladium(II) dichloride (380 mg, 0.54 mmol) in DMF (20 mL) was heated to 85° C. for 4 hours. Standard work-up and purification afforded 4-(5-vinyl-pyrimidin-2-ylamino)-benzoic acid methyl ester (321 mg, 25% over 2 steps).

To a solution of the above pyrimidine (150 mg, 0.58 mmol) in THF (5 mL) was added NaH (30 mg, 0.70 mmol) and the mixture was stirred at room temperature for 30 minutes. A solution of MeI (0.12 mL) in THF (0.5 mL) was added and the mixture stirred for an additional 1.2 hours at room temperature. Standard work-up and purification afforded 4-[methyl-(5-vinyl-pyrimidin-2-yl)-amino]-benzoic acid methyl ester (103 mg, 66%).

To AD-mix-α (530 mg) was added a solution of tert-butanol (1.9 mL) and H₂O (0.4 mL) and the mixture was stirred at room temperature for 15 minutes. A solution of the above substrate (103 mg, 0.383 mmol) in THF (0.5 mL) was added followed by OsO₄ (2.5%, 0.1 mL) and the mixture stirred at room temperature overnight. A second aliquot of OsO₄ (0.16 mL) was added followed by H₂O (4 drops) and the mixture stirred overnight. Standard work-up afforded the desired intermediate. To a solution of the diol in acetone (2 mL) was added a solution of NaIO₄ (138 mg, 0.648 mmol) H₂O (1 mL) and the mixture was stirred at room temperature for 2 hours. Standard work-up and purification afforded 4-[(5-formyl-pyrimidin-2-yl)-methyl-amino]-benzoic acid methyl ester (68 mg, 66%).

COMPOUND 285 was isolated as a yellow solid (68 mg, 45% over 2 steps). ¹H NMR (CDCl₃) δ 1.64-1.91 (m, 7H), 2.28 (s, 3H), 2.49-2.73 (m, 3H), 3.17-3.25 (m, 2H), 3.44-3.52 (m, 6H), 3.68-3.74 (m, 1H), 3.88-4.10 (m, 6H), 4.89 (dd, 1H, J=9.0, 6.3 Hz), 6.85-6.91 (m, 1H), 7.01-7.03 (m, 1H), 7.13 (d, 1H, J=8.1 Hz), 7.34 (d, 1H, J=8.4 Hz), 8.00 (d, 2H, J=8.1 Hz), 8.46 (br s, 2H); ¹³C NMR (CDCl₃) δ 20.79, 29.81, 30.13, 38.41, 46.84, 48.92, 49.32, 51.22, 53.44, 67.13, 67.21, 115.84, 116.12, 125.69, 127.85, 128.88, 130.84, 134.47, 148.80, 156.93, 159.54, 160.20, 161.51, 168.63; ES-MS m/z 603 (M+1). Anal. Calcd. for C₃₃H₃₉FN₆O₄.0.89CH₂Cl₂.0.77CH₄O: C, 59.22; H, 6.29; N, 11.95. Found: C, 59.16; H, 6.43; N, 12.14.

### EXAMPLE 286

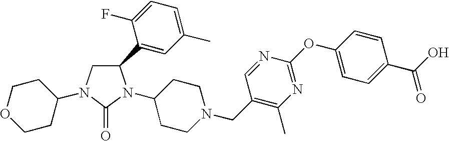

### COMPOUND 286: 4-(5-{4-[(R)-5-(2-Fluoro-5-methyl-phenyl)-2-oxo-3-(tetrahydro-pyran-4-yl)-imidazolidin-1-yl]-piperidin-1-ylmethyl}-4-methyl-pyrimidin-2-yloxy)-benzoic acid

COMPOUND 286 was isolated as a white solid (77 mg, 54% over 2 steps). ¹H NMR (CDCl₃) δ 1.55-1.87 (m, 8H), 2.16-2.27 (m, 1H), 2.25 (s, 3H), 2.49-2.52 (m, 1H), 2.53 (s, 3H), 3.02-3.17 (m, 3H), 3.43-3.51 (m, 2H), 3.66-3.76 (m, 4H), 3.98-4.05 (m, 3H), 4.90 (dd, 1H, J=9.3, 6.6 Hz), 6.81-6.87 (m, 1H), 6.96-7.01 (m, 1H), 7.13 (d, 1H, J=6.6 Hz), 7.21 (d, 2H, J=8.7 Hz), 8.04 (d, 2H, J=8.7 Hz), 8.42 (br s, 1H); ¹³C NMR (CDCl₃) δ 21.14, 22.72, 25.99, 30.23, 30.50, 47.46, 49.23, 52.89, 53.82, 67.53, 67.61, 68.34, 115.93, 116.21, 121.77, 127.94, 128.97, 130.93, 132.08, 134.68, 157.02, 160.14, 164.50, 169.14, 171.25; ES-MS m/z 604 (M+1). Anal. Calcd. for C₃₃H₃₈FN₅O₅.0.6CH₂Cl₂.0.39CH₄O: C, 61.16; H, 6.16; N, 10.49. Found: C, 61.18; H, 6.12; N, 10.43.

### EXAMPLE 287

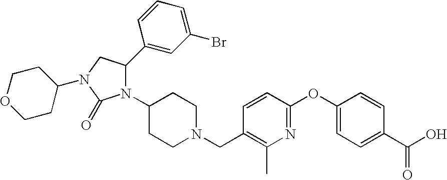

### COMPOUND 287: 4-(5-{4-[5-(3-Bromo-phenyl)-2-oxo-3-(tetrahydro-pyran-4-yl)-imidazolidin-1-yl]-piperidin-1-ylmethyl}-6-methyl-pyridin-2-yloxy)-benzoic acid

4-(3-Bromo-phenyl)-3-piperidin-4-yl-1-(tetrahydro-pyran-4-yl)-imidazolidin-2-one was prepared using the same chemistry as 4-(2-fluoro-5-methyl-phenyl)-3-piperidin-4-yl-1-(tetrahydro-pyran-4-yl)-imidazolidin-2-one (see Intermediates) except that 3-bromobenzaldehyde was used in lieu of 3-fluoro-5-methylbenzaldehyde. ¹H NMR (CDCl₃) δ 1.01-1.15 (m, 1H), 1.47 (d, 1H, J=12.3 Hz), 1.59-1.76 (m, 7H), 2.45-2.65 (m, 2H), 2.91 (d, 1H, J=12.3 Hz), 2.99-3.09 (m, 2H), 3.43-3.51 (m, 2H), 3.64 (d, 1H, J=9.0 Hz), 3.72 (m, 1H), 3.96-4.08 (m, 3H), 4.56 (dd, 1H, J=9.3, 6.6 Hz), 7.18-7.27 (m, 2H), 7.43 (d, 1H, J=7.2 Hz), 7.47 (s, 1H).

COMPOUND 287 was isolated as a pale brown powder (21.5 mg, 28%). ¹H NMR (CDCl₃) δ 1.50-1.75 (m, 5H), 1.87 (d, 2H, J=11.1 Hz), 2.43 (s, 3H), 2.47-2.75 (m, 2H), 3.09 (dd, 1H, J=9.0, 6.0 Hz), 3.20 (br s, 1H), 3.43-3.52 (m, 3H), 3.71 (t, 1H, J=9.0 Hz), 3.91-4.13 (m, 6H), 4.65 (t, 1H, J=7.5 Hz), 6.75 (d, 1H, J=7.5 Hz), 7.13 (d, 2H, J=7.5 Hz), 7.16 (m, 2H), 7.27 (m, 1H), 7.37 (d, 1H, J=7.5 Hz), 7.49 (s, 1H), 8.02 (d, 2H, J=7.5 Hz); ¹³C NMR (CDCl₃) δ 22.8, 26.7, 28.7, 30.4, 48.7, 49.3, 50.0, 52.3, 52.6, 55.3, 57.0, 67.5, 110.1, 120.8, 123.5, 126.0, 127.2, 129.6, 131.2, 132.1, 132.2, 144.2, 145.0, 157.5, 158.4, 160.1, 162.7, 169.4; ES-MS m/z 649 (M+H). Anal. Calcd. for C₃₃H₃₇N₄O₅Br.1.2CH₂Cl₂: C, 54.66; H, 5.28; N, 7.46. Found: C, 54.84; H, 5.27; N, 7.41.

### EXAMPLE 288

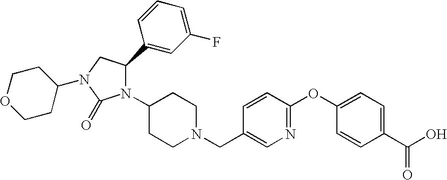

### COMPOUND 288: 4-(5-{4-[(R)-5-(3-Fluoro-phenyl)-2-oxo-3-(tetrahydro-pyran-4-yl)-imidazolidin-1-yl]-piperidin-1-ylmethyl}-pyridin-2-yloxy)-benzoic acid

COMPOUND 288 was isolated as a white solid (118 mg, 61% over 2 steps). ¹H NMR (CD₃OD) δ 1.54 (dq, 1H, J=12.6, 3.9 Hz), 1.68 (m, 4H), 1.80 (d, 2H, J=12.0 Hz), 2.15 (dq, 1H, J=12.3, 3.6 Hz), 2.39 (q, 2H, J=12.0 Hz), 3.04 (d, 1H, J=12.9 Hz), 3.17 (m, 2H), 3.51 (t, 2H, J=12.0 Hz), 3.60 (tt, 1H, J=12.0, 3.6 Hz), 3.77 (s, 2H), 3.83 (t, 1H, J=9.3 Hz), 3.97 (m, 3H), 4.80 (m, 1H), 7.07 (d, 1H, J=8.4 Hz), 7.11 (t, 1H, J=8.4 Hz), 7.18 (m, 1H), 7.19 (d, 2H, J=8.4 Hz), 7.25 (d, 1H, J=7.8 Hz), 7.43 (q, 1H, J=6.9 Hz), 7.86 (dd, 1H, J=8.4, 2.4 Hz), 8.09 (d, 2H, J=8.7 Hz), 8.13 (d, 1H, J=2.1 Hz); ES-MS m/z 575 (M+H).

### EXAMPLE 289

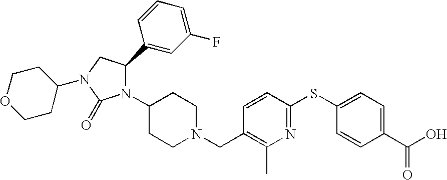

### COMPOUND 289: 4-(5-{4-[(R)-5-(3-Fluoro-phenyl)-2-oxo-3-(tetrahydro-pyran-4-yl)-imidazolidin-1-yl]-piperidin-1-ylmethyl}-6-methyl-pyridin-2-ylsulfanyl)-benzoic acid

COMPOUND 289 was isolated as a white solid (40 mg, 44% over 2 steps). ¹H NMR (CD₃OD) δ 1.60-1.95 (m, 8H), 2.30 (dq, 1H, J=12.3, 3.6 Hz), 2.55 (s, 3H), 2.80 (q, 2H, J=12.0 Hz), 3.18 (m, 1H), 3.26 (d, 1H, J=11.4 Hz), 3.51 (t, 2H, J=11.5 Hz), 3.62 (tt, 1H, J=12.6, 3.6 Hz), 3.83 (t, 1H, J=9.3 Hz), 3.97 (m, 3H), 4.06 (s, 2H), 4.79 (m, 1H), 6.97 (d, 1H, J=8.1 Hz), 7.12 (dt, 1H, J=8.7, 2.4 Hz), 7.18 (d, 1H, J=9.6 Hz), 7.26 (d, 1H, J=7.5 Hz), 7.44 (q, 1H, J=6.9 Hz), 7.63 (m, 3H), 8.08 (d, 2H, J=8.4 Hz); ES-MS m/z 605 (M+H).

### EXAMPLE 290

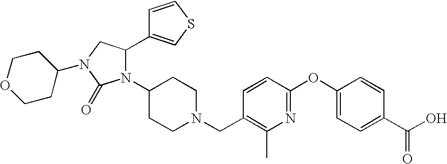

### COMPOUND 290: 4-(6-Methyl-5-{4-[2-oxo-3-(tetrahydro-pyran-4-yl)-5-thiophen-3-yl-imidazolidin-1-yl]-piperidin-1-ylmethyl}-pyridin-2-yloxy)-benzoic acid

3-Piperidin-4-yl-1-(tetrahydro-pyran-4-yl)-4-thiophen-3-yl-imidazolidin-2-one was prepared using the same chemistry as 4-(2-fluoro-5-methyl-phenyl)-3-piperidin-4-yl-1-(tetrahydro-pyran-4-yl)-imidazolidin-2-one (see Intermediates) except that 3-thiophenecarboxaldehyde was used in lieu of 3-fluoro-5-methylbenzaldehyde. ¹H NMR (CDCl₃) δ 1.08-1.22 (m, 1H), 1.49 (d, 1H, J=12.3 Hz), 1.64-1.70 (m, 5H), 1.78-1.92 (m, 1H), 2.50-2.68 (m, 2H), 2.78 (br s, 1H), 2.96 (d, 1H, J=12.8 Hz), 3.09-3.14 (m, 2H), 3.40-3.53 (m, 2H), 3.59 (t, 1H, J=9.0 Hz), 3.72 (m, 1H), 3.98-4.08 (m, 3H), 4.75 (dd, 1H, J=9.0, 6.6 Hz), 7.07 (dd, 1H, J=5.1, 1.2 Hz), 7.21 (dd 1H, J=3.0, 1.2 Hz), 7.32 (dd, 1H, J=4.8, 3.0 Hz).

COMPOUND 290 was isolated as a white solid (27.9 mg, 29% over 2 steps). ¹H NMR (CD₃OD) δ 1.29 (m, 1H), 1.65 (m, 2H), 1.70-1.90 (m, 6H), 2.34 (m, 1H), 2.46 (s, 3H), 2.89 (m, 2H), 3.17-3.25 (m, 2H), 3.48 (t, 2H, J=11.4 Hz), 3.64 (br s, 1H), 3.73 (t, 1H, J=8.7 Hz), 3.98 (m, 3H), 4.11 (s, 2H), 6.83 (d, 1H, J=8.1 Hz), 7.14 (br s, 3H), 7.45 (br s, 2H), 7.85 (d, 1H, J=8.1 Hz), 8.06 (s, 2H); ¹³C NMR (CD₃OD) δ 22.5, 28.4, 28.9, 31.3, 31.5, 50.8, 51.6, 53.7, 53.8, 54.1, 58.1, 68.6, 68.7, 110.9, 121.5, 122.0, 125.0, 127.1, 129.0, 132.9, 144.2, 145.5, 158.9, 159.7, 164.6; ES-MS m/z 577 (M+H). Anal. Calcd. for C₃₁H₃₆N₄O₂S.2.7CH₂Cl₂.4.5H₂O: C, 45.63; H, 5.73; N, 6.32. Found: C, 45.72; H, 5.72; N, 6.39.

### EXAMPLE 291

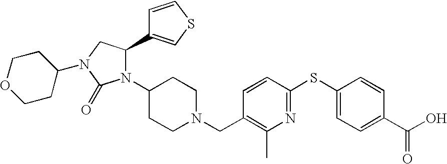

### COMPOUND 291: 4-(6-Methyl-5-{4-[(R)-2-oxo-3-(tetrahydro-pyran-4-yl)-5-thiophen-3-yl-imidazolidin-1-yl]-piperidin-1-ylmethyl}-pyridin-2-ylsulfanyl)-benzoic acid

COMPOUND 291 was isolated as a white solid (1.41 g, 81% over 2 steps). ¹H NMR (CDCl₃) δ 1.47-1.81 (m, 7H), 2.08 (m, 2H), 2.30-2.61 (m, 6H), 3.12-3.23 (m, 2H), 3.41-3.52 (m, 3H), 3.61 (t, 2H, J=9.0 Hz), 3.88-4.09 (m, 6H), 4.73 (t, 1H, J=7.6 Hz), 6.50 (br s, 1H), 7.00 (d, 1H, J=5.2 Hz), 7.16-7.23 (m, 2H), 7.62 (d, 2H, J=7.9 Hz), 8.04 (d, 2H, J=7.9 Hz); ¹³C NMR (CDCl₃) δ 20.96, 22.43, 26.38, 28.01, 29.91, 30.05, 47.36, 48.83, 49.82, 51.21, 52.05, 56.37, 67.12, 67.19, 77.23, 119.11, 122.00, 123.20, 125.31, 127.47, 130.95, 134.71, 134.82, 140.37, 142.44, 157.59, 159.44, 161.62, 170.09, 175.10; ES-MS m/z 593 (M+H). Anal. Calcd. for C₃₁H₃₆N₄O₄S₂.1.1H₂O.1.1CH₃CO₂H: C, 58.76; H, 6.33; N, 8.26; S, 9.45. Found: C, 58.76; H, 6.26; N, 8.23; S, 9.47.

### EXAMPLE 292

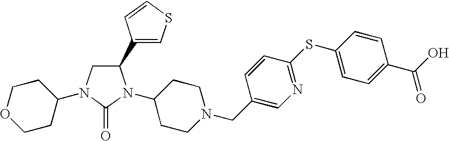

### COMPOUND 292: 4-(5-{4-[(R)-2-Oxo-3-(tetrahydro-pyran-4-yl)-5-thiophen-3-yl-imidazolidin-1-yl]-piperidin-1-ylmethyl}-pyridin-2-ylsulfanyl)-benzoic acid

COMPOUND 292 was isolated as a white solid (138 mg, 80% over 2 steps). ¹H NMR (CD₃OD) δ 1.57-1.82 (m, 7H), 2.16-2.30 (m, 1H), 2.46-2.58 (m, 2H), 3.09-3.13 (m, 1H), 3.19-3.25 (m, 2H), 3.48-3.64 (m, 3H), 3.74 (t, 1H, J=9.0 Hz), 3.86 (s, 2H), 3.91-4.03 (m, 3H), 4.88-4.92 (m, 1H), 7.11 (d, 1H, J=8.4 Hz), 7.15 (dd, 1H, J=4.8, 0.9 Hz), 7.43-7.44 (m, 1H), 7.48 (dd, 1H, J=4.8, 3.0 Hz), 7.63 (d, 2H, J=8.4 Hz), 7.69 (dd, 1H, J=8.4, 2.4 Hz), 8.08 (d, 2H, J=8.4 Hz), 8.40 (d, 1H, J=1.8 Hz); ES-MS m/z 579 (M+1). Anal. Calcd. for C₃₀H₃₄N₄O₄S₂.0.7CH₂Cl₂: C, 57.78; H, 5.59; N, 8.78. Found: C, 57.49; H, 5.85; N, 8.73.

### EXAMPLE 293

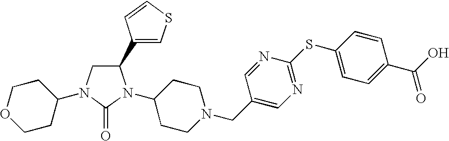

### COMPOUND 293: 4-(5-{4-[(R)-2-Oxo-3-(tetrahydro-pyran-4-yl)-5-thiophen-3-yl-imidazolidin-1-yl]-piperidin-1-ylmethyl}-pyrimidin-2-ylsulfanyl)-benzoic acid

COMPOUND 293 was isolated as a white solid (48 mg, 56% over 2 steps). ¹H NMR (CD₃OD) δ 1.34-1.82 (m, 7H), 2.05-2.26 (m, 3H), 2.91-2.94 (m, 1H), 3.02-3.06 (m, 1H), 3.21 (dd, 1H, J=8.7, 6.6 Hz), 3.48-3.61 (m, 5H), 3.73 (t, 1H, J=9.0 Hz), 3.92-4.03 (m, 3H), 4.88-4.92 (m, 1H), 7.14 (dd, 1H, J=5.1, 1.2 Hz), 7.41 (dd, 1H, J=2.7, 1.2 Hz), 7.46 (dd, 1H, J=5.1, 3.0 Hz), 7.69-7.72 (m, 2H), 8.06-8.09 (m, 2H), 8.49 (s, 2H); ES-MS m/z 580 (M+1). Anal. Calcd. for C₂₉H₃₃N₅O₄S₂.0.3CH₂Cl₂: C, 58.15; H, 5.60; N, 11.57. Found: C, 58.48; H, 5.75; N, 11.40.

### EXAMPLE 294

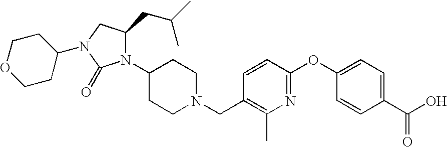

### COMPOUND 294: 4-(5-{4-[(R)-5-Isobutyl-2-oxo-3-(tetrahydro-pyran-4-yl)-imidazolidin-1-yl]-piperidin-1-ylmethyl}-6-methyl-pyridin-2-yloxy)-benzoic acid

COMPOUND 294 was isolated as a white solid (36 mg, 56% over 2 steps). ¹H NMR (CD₃OD) δ 1.00 (t, 6H, J=5.1 Hz), 1.45 (t, 1H, J=10.2 Hz), 1.55-1.90 (m, 7H), 2.00 (dq, 1H, J=12.0, 3.6 Hz), 2.18 (dq, 1H, J=12.0, 3.6 Hz), 2.35 (q, 2H, J=12.0 Hz), 2.52 (s, 3H), 3.03 (m, 1H), 3.12 (t, 2H, J=10.8 Hz), 3.50 (m, 5H), 3.72 (s, 2H), 3.75 (m, 1H), 3.81 (m, 1H), 4.01 (dd, 2H, J=11.4, 3.6 Hz), 6.82 (d, 1H, J=8.4 Hz), 7.15 (d, 2H, J=8.7 Hz), 7.80 (d, 1H, J=8.1 Hz), 8.08 (d, 2H, J=8.7 Hz); ¹³C NMR (CD₃OD) δ 20.76, 20.90, 23.60, 24.92, 28.12, 29.83, 29.97, 30.17, 44.16, 44.89, 49.36, 51.17, 51.79, 53.09, 53.87, 57.83, 67.26, 67.34, 109.25, 119.74 (2C), 124.91, 131.61 (2C), 143.36, 157.66, 158.31, 160.75, 162.26, 170.33; ES-MS m/z 551 (M+H). Anal. Calcd. for C₃₁H₄₂N₄O₅.0.3CH₂Cl₂: C, 65.25; H, 7.45; N, 9.72. Found: C, 65.30; H, 7.70; N, 9.87.

### EXAMPLE 295

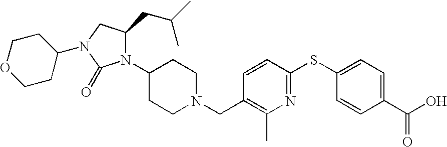

### COMPOUND 295: 4-(5-{4-[(R)-5-Isobutyl-2-oxo-3-(tetrahydro-pyran-4-yl)-imidazolidin-1-yl]-piperidin-1-ylmethyl}-6-methyl-pyridin-2-ylsulfanyl)-benzoic acid

COMPOUND 295 was isolated as a white solid (45 mg, 55% over 2 steps). ¹H NMR (CD₃OD) δ 1.01 (t, 6H, J=5.1 Hz), 1.45 (t, 1H, J=10.2 Hz), 1.55-1.90 (m, 6H), 2.00 (q, 2H, J=12.0 Hz), 2.29 (q, 1H, J=12.0 Hz), 2.56 (q, 1H, J=12.0 Hz), 2.64 (s, 3H), 3.06 (m, 1H), 3.22 (q, 2H, J=10.8 Hz), 3.40-3.65 (m, 6H), 3.74 (m, 1H), 3.85 (m, 1H), 4.01 (dd, 2H, J=11.4, 3.6 Hz), 4.39 (s, 2H), 7.02 (d, 1H, J=8.1 Hz), 7.71 (d, 2H, J=8.1 Hz), 7.77 (d, 1H, J=8.4 Hz), 8.12 (d, 2H, J=8.1 Hz); ES-MS m/z 567 (M+H).

### EXAMPLE 296

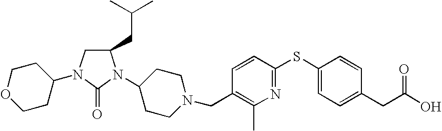

### COMPOUND 296: [4-(5-{4-[(R)-5-Isobutyl-2-oxo-3-(tetrahydro-pyran-4-yl)-imidazolidin-1-yl]-piperidin-1-ylmethyl}-6-methyl-pyridin-2-ylsulfanyl)-phenyl]-acetic acid

COMPOUND 296 was isolated as a white foam (153 mg, 54% over 2 steps). ¹H NMR (CD₃OD) δ 0.94-0.98 (m, 6H), 1.37-1.43 (m, 1H), 1.58-1.82 (m, 8H), 1.95-1.99 (m, 1H), 2.14-2.18 (m, 1H), 2.40-2.43 (m, 2H), 2.54 (s, 3H), 2.97-3.02 (m, 1H), 3.07-3.10 (m, 2H), 3.48-3.50 (m, 5H), 3.64 (s, 2H), 3.70 (s, 3H), 3.75 (m, 1H), 3.95-3.99 (m, 2H), 6.70 (d, 1H, J=7.8 Hz), 7.41 (d, 2H, J=8.1 Hz), 7.48-7.53 (m, 3H).

### EXAMPLE 297

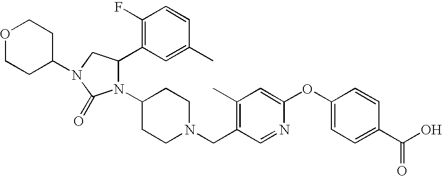

### COMPOUND 297: 4-(5-{4-[5-(2-Fluoro-5-methyl-phenyl)-2-oxo-3-(tetrahydro-pyran-4-yl)-imidazolidin-1-yl]-piperidin-1-ylmethyl}-4-methyl-pyridin-2-yloxy)-benzoic acid

COMPOUND 297 was isolated as a white solid (54.3 mg, 63% over 2 steps). ¹H NMR (CDCl₃) δ 1.25-1.74 (m, 8H), 1.98-2.13 (m, 2H), 2.21 (s, 3H), 2.32 (s, 3H), 2.89 (d, 1H, J=9.9 Hz), 3.08 (t, 2H, J=6.9 Hz), 3.45-3.50 (m, 4H), 3.67 (t, 1H, J=9.3 Hz), 3.77 (t, 1H, J=11.7 Hz), 3.98-4.14 (m, 3H), 4.91 (t, 1H, J=7.6 Hz), 6.72 (s, 1H), 6.78 (t, 1H, J=9.0 Hz), 6.92-6.96 (m, 1H), 7.11 (d, 3H, J=7.5 Hz), 7.97 (s, 1H), 8.01 (d, 2H, J=8.1 Hz); ¹³C NMR (CDCl₃) δ 19.57, 21.01, 28.50, 30.18, 30.36, 47.54, 49.04, 51.67, 52.95, 53.72, 56.59, 67.45, 67.52, 113.30, 115.61, 115.90, 120.44, 127.72 (d, J=180 Hz), 127.83, 128.66, 130.47, 130.57, 132.06, 134.40, 149.45, 151.74, 156.87, 158.47, 160.18, 162.96, 169.69; ES-MS m/z 603 (M+H). Anal. Calcd. for C₃₄H₃₉FN₄O₅.1.2CH₂Cl₂: C, 60.00; H, 5.92; N, 7.95. Found: C, 59.98; H, 5.84; N, 7.88.

Examples 298 to 303 were prepared following the scheme illustrated below. RCHO is as defined in the table and Y is as defined in the individual examples.

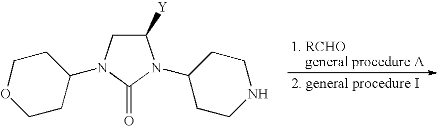

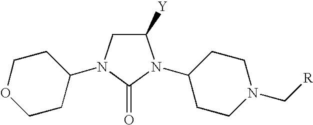

| Example | RCHO |
|---|---|
| 298 | 4-(5-formyl-6-methyl-pyridin-2-yloxy)-3-methyl-benzonitrile |
| 299 | 4-(5-formyl-4-methyl-pyrimidin-2-ylamino)-benzonitrile |
| 300 | 4-(5-formyl-pyridin-2-yloxy)-3-methyl-benzonitrile |
| 301 | 4-(5-formyl-6-methyl-pyridin-2-yloxy)-3-methyl-benzonitrile |
| 302 | 4-(5-formyl-4-methyl-pyrimidin-2-ylamino)-benzonitrile |
| 303 | 4-(5-formyl-4-methyl-pyrimidin-2-ylamino)-benzonitrile |

### EXAMPLE 298

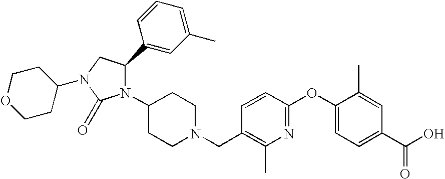

### COMPOUND 298: 3-Methyl-4-(6-methyl-5-{4-[(R)-2-oxo-3-(tetrahydro-pyran-4-yl)-5-m-tolyl-imidazolidin-1-yl]-piperidin-1-ylmethyl}-pyridin-2-yloxy)-benzoic acid

COMPOUND 298 was isolated as an orange powder (33.1 mg, 45% over 2 steps). ¹H NMR (CDCl₃) δ 1.25-1.40 (m, 1H), 1.40-1.53 (m, 2H), 1.66 (br s, 3H), 1.82 (br s, 1H), 2.21 (s, 3H), 2.30 (s, 3H), 2.38 (s, 3H), 2.35-2.60 (m, 4H), 3.11 (br s, 2H), 3.35-3.53 (m, 3H), 3.67 (m, 2H), 3.75-4.22 (m, 4H), 4.60 (br s, 1H), 6.63 (br s, 1H), 6.75-7.20(m, 4H), 7.87 (br s, 1H), 7.97-8.16 (m, 2H), 8.06 (s, 1H); ¹³C NMR (CDCl₃) δ 16.8, 21.8, 22.7, 30.3, 30.5, 48.8, 49.2, 53.8, 67.6, 121.2, 124.1, 127.7, 129.4, 130.8, 139.2, 157.4, 160.2; ES-MS m/z 599 (M+H). Anal. Calcd. for C₃₅H₄₂N₄O₅.1.5CH₂Cl₂.0.3CH₃OH: C, 60.08; H, 6.33; N, 7.61. Found: C, 60.16; H, 6.17; N, 7.25.

### EXAMPLE 299

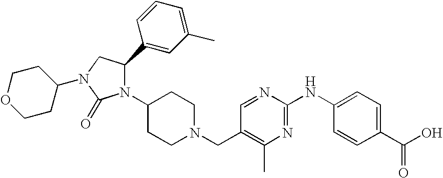

### COMPOUND 299: 4-(4-Methyl-5-{4-[(R)-2-oxo-3-(tetrahydro-pyran-4-yl)-5-m-tolyl-imidazolidin-1-yl]-piperidin-1-ylmethyl}-pyrimidin-2-ylamino)-benzoic acid

COMPOUND 299 was isolated as a brown solid (49 mg, 29% over 2 steps). ¹H NMR (CD₃OD) δ 1.39 (dq, 1H, J=12.0, 3.6 Hz), 1.54 (d, 1H, J=12.0 Hz), 1.60-1.85 (m, 5H), 2.10 (m, 3H), 2.38 (s, 3H), 2.47 (s, 3H), 2.85 (d, 1H, J=11.1 Hz), 2.99 (d, 1H, J=11.1 Hz), 3.15 (m, 1H), 3.40-3.65 (m, 5H), 3.78 (t, 1H, J=9.0 Hz), 3.97 (m, 3H), 4.72 (m, 1H), 7.20 (m, 3H), 7.28 (m, 1H), 7.82 (d, 2H, J=9.0 Hz), 7.96 (d, 2H, J=8.7 Hz), 8.21 (s, 1H); ES-MS m/z 585 (M+H).

### EXAMPLE 300

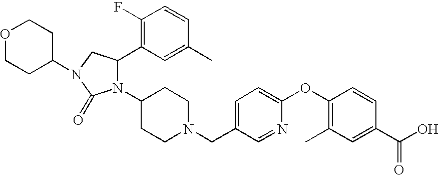

### COMPOUND 300: 4-(5-{4-[5-(2-Fluoro-5-methyl -phenyl)-2-oxo-3-(tetrahydro-pyran-4-yl)-imidazolidin-1-yl]-piperidin-1-ylmethyl}-pyridin-2-yloxy)-3-methyl-benzoic acid

COMPOUND 300 was isolated as a yellow solid (55.3 mg, 69% over 2 steps). ¹H NMR (CDCl₃) δ 1.35-1.74 (m, 4H), 2.00-2.30 (m, 4H), 2.99-3.99 (m, 14H), 4.90 (t, 1H, J=0.9 Hz), 6.76-7.12 (m, 5H), 7.66-8.01 (m, 4H); ¹³C NMR (CDCl₃) δ 16.76, 21.12, 28.03, 29.80, 30.27, 30.47, 47.59, 49.16, 51.33, 52.59, 58.62, 67.62, 110.98, 115.82, 116.10, 121.50, 128.77, 129.48, 130.65, 133.64, 134.52, 142.06, 149.47, 156.03, 156.99, 160.18, 163.53, 170.07; ES-MS m/z 603 (M+H). Anal. Calcd. for C₃₄H₃₉FN₄O₅.1.9CH₂Cl₂: C, 56.43; H, 5.65; N, 7.33. Found: C, 56.39; H, 5.56; N, 7.29.

### EXAMPLE 301

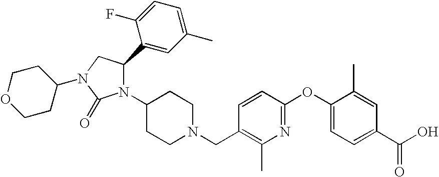

### COMPOUND 301: 4-(5-{4-[(R)-5-(2-Fluoro-5-methyl-phenyl)-2-oxo-3-(tetrahydro-pyran-4-yl)-imidazolidin-1-yl]-piperidin-1-ylmethyl}-6-methyl-pyridin-2-yloxy)-3-methyl-benzoic acid

COMPOUND 301 was isolated as a pale orange powder (34.2 mg, 44% over 2 steps). ¹H NMR (CDCl₃) δ 1.23-1.50 (m, 1H), 1.65 (br s, 3H), 1.75-1.82 (m, 2H), 2.20 (s, 3H), 2.25 (s, 3H), 2.39 (s, 3H), 2.50-3.00 (m, 3H), 3.15 (br s, 2H), 3.45 (m, 3H), 3.69 (br s, 2H), 3.75-3.90 (m, 2H), 3.99 (m, 4H), 4.89 (br s, 1H), 6.60 (br s, 1H), 6.84-7.12 (m, 5H), 7.84-7.98 (m, 2H); ¹³C NMR (CDCl₃) δ 16.3, 20.7, 22.2, 26.8, 29.8, 30.0, 46.9, 48.8, 51.9, 53.4, 67.0, 67.1, 108.3, 115.7 (d, J=20.2 Hz), 120.7, 127.6, 127.7, 128.6, 129.0, 130.2, 130.5 (d, J=7.5 Hz), 133.2, 134.2, 143.3, 143.4, 156.0, 156.9, 158.3 (d, J=242 Hz), 159.6, 162.1; ES-MS m/z 617 (M+H). Anal. Calcd. for C₃₅H₄₁N₄O₅F.1.7CH₂Cl₂: C, 57.92; H, 5.88; N, 7.36. Found: C, 57.83; H, 5.91; N, 7.26.

### EXAMPLE 302

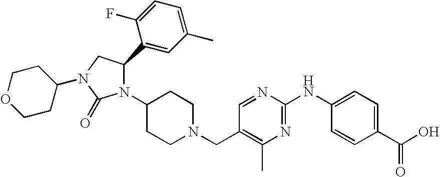

### COMPOUND 302: 4-(5-{4-[(R)-5-(2-Fluoro-5-methyl-phenyl)-2-oxo-3-(tetrahydro-pyran-4-yl)-imidazolidin-1-yl]-piperidin-1-ylmethyl}-4-methyl-pyrimidin-2-ylamino)-benzoic acid

COMPOUND 302 was isolated as a brown solid (52 mg, 30% over 2 steps). ¹H NMR (CD₃OD) δ 1.39 (dq, 1H, J=12.0, 3.6 Hz), 1.58 (d, 1H, J=12.0 Hz), 1.60-1.85 (m, 5H), 2.02 (dq, 1H, J=12.0, 3.6 Hz), 2.20 (m, 2H), 2.35 (s, 3H), 2.49 (s, 3H), 2.89 (d, 1H, J=11.4 Hz), 3.03 (d, 1H, J=10.8 Hz), 3.24 (m, 1H), 3.40-3.65 (m, 5H), 3.83 (t, 1H, J=9.0 Hz), 3.99 (m, 3H), 5.03 (m, 1H), 7.04 (m, 1H), 7.20 (m, 1H), 7.24 (d, 1H, J=9.0 Hz), 7.85 (d, 2H, J=9.0 (d, 2H, J=9.0 Hz), 8.23 (s, 1H); ES-MS m/z 603 (M+H).

### EXAMPLE 303

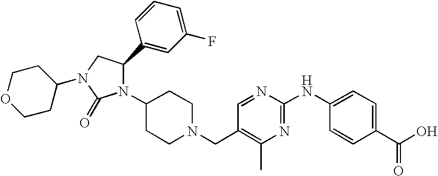

### COMPOUND 303: 4-(5-{4-[(R)-5-(3-Fluoro-phenyl)-2-oxo-3-(tetrahydro-1-pyran-4-yl)-imidazolidin-1-yl]-piperidin-1-ylmethyl}-4-methyl-pyrimidin-2-ylamino)-benzoic acid

COMPOUND 303 was isolated as a beige solid (46 mg, 31% over 2 steps). ¹H NMR (CD₃OD) δ 1.36 (m, 1H), 1.57 (d, 1H, J=11.1 Hz), 1.60-1.85 (m, 5H), 2.04 (dq, 1H, J=12.0, 3.6 Hz), 2.12 (q, 2H, J=12.0 Hz), 2.48 (s, 3H), 2.88 (d, 1H, J=11.7 Hz), 3.01 (d, 1H, J=10.2 Hz), 3.15 (m, 1H), 3.40-3.65 (m, 6H), 3.81 (t, 1H, J=9.0 Hz), 3.96 (m, 3H), 4.80 (m, 1H), 7.10 (t, 1H, J=8.4 Hz), 7.16 (d, 1H, J=9.6 Hz), 7.24 (d, 1H, J=7.8 Hz), 7.43 (q, 1H, J=7.2 Hz), 7.84 (d, 2H, J=8.7 Hz), 7.96 (d, 2H, J=8.7 Hz), 8.22 (s, 1H); ES-MS m/z 589 (M+H).

Examples 304 to 311 were prepared following the scheme illustrated below. RCHO is as defined in the table and Y is as defined in the individual examples.

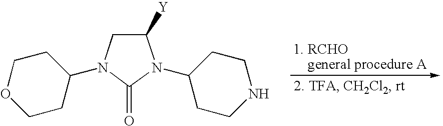

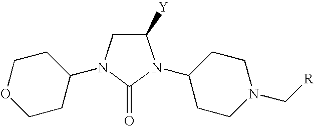

| Example | RCHO |
|---|---|
| 304 | [4-(5-formyl-pyridin-2-ylsulfanyl)-phenoxy]- |
| | acetic acid tert-butyl ester |
| 305 | 4-(6-ethyl-5-formyl-pyridin-2-yloxy)-benzoic acid tert- |
| | butyl ester |
| 306 | 4-(5-formyl-6-methyl-pyridin-2-yloxy)-benzoic acid tert- |
| | butyl ester |
| 307 | [4-(5-formyl-pyridin-2-ylsulfanyl)-phenoxy]- |
| | acetic acid tert-butyl ester |
| 308 | 4-(6-ethyl-5-formyl-pyridin-2-yloxy)-benzoic acid tert- |
| | butyl ester |
| 309 | [4-(5-formyl-6-methyl-pyridin-2-yloxy)-phenoxy]- |
| | acetic acid tert-butyl ester |
| 310 | [4-(5-formyl-6-methyl-pyridin-2-ylsulfanyl)-phenoxy]- |
| | acetic acid tert-butyl ester |
| 311 | [4-(5-formyl-6-methyl-pyridin-2-yloxy)-phenylsulfanyl]- |
| | acetic acid tert-butyl ester |

### EXAMPLE 304

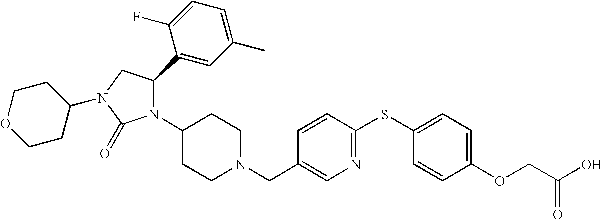

### COMPOUND 304: [4-(5-{4-[(R)-5-(2-Fluoro-5-methyl-phenyl)-2-oxo-3-(tetrahydro-pyran-4-yl)-imidazolidin-1-yl]-piperidin-1-ylmethyl}-pyridin-2-ylsulfanyl)-phenoxy]-acetic acid

COMPOUND 304 was isolated as a white solid (62 mg, 68% over 2 steps). ¹H NMR (CD₃OD) δ 1.70 (m, 2H), 1.80 (m, 3H), 1.95 (d, 2H, J=14.1 Hz), 2.36 (m, 1H), 2.36 (s, 3H), 3.03 (m, 2H), 3.28 (t, 1H, J=8.0 Hz), 3.48 (m, 2H), 3.52 (t, 2H, J=11.4 Hz), 3.64 (t, 1H, J=9.6 Hz), 3.86 (t, 1H, J=9.3 Hz), 3.93 (m, 1H), 4.00 (m, 2H), 4.24 (s, 2H), 4.80 (s, 2H), 5.02 (m, 1H), 6.93 (d, 1H, J=8.4 Hz), 7.07 (m, 1H), 7.12 (dd, 2H, J=8.7, 2.3 Hz), 7.22 (m, 1H), 7.27 (br d, 1H, J=7.5 Hz), 7.57 (dd, 2H, J=8.7, 2.3 Hz), 7.66 (dd, 1H, J=8.4, 2.4 Hz), 8.42 (s, 1H); ES-MS m/z 635 (M+H).

### EXAMPLE 305

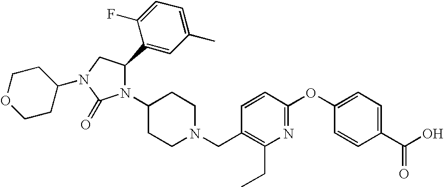

### COMPOUND 305: 4-(6-Ethyl-5-{4-[(R)-5-(2-fluoro-5-methyl-phenyl)-2-oxo-3-(tetrahydro-pyran-4-yl)-imidazolidin-1-yl]-piperidin-1-ylmethyl}-pyridin-2-yloxy)-benzoic acid

COMPOUND 305 was isolated as a white solid (41 mg, 54% over 2 steps). ¹H NMR (CD₃OD) δ 1.15 (t, 3H, J=6.9 Hz), 1.43 (dq, 1H, J=12.0, 3.6 Hz), 1.55-1.85 (m, 6H), 2.06 (dq, 1H, J=12.0, 3.6 Hz), 2.34 (q, 2H, J=12.0 Hz), 2.34 (s, 3H), 2.75 (q, 2H, J=7.5 Hz), 2.94 (d, 1H, J=8.7 Hz), 3.09 (d, 1H, J=9.6 Hz), 3.25 (m, 1H), 3.52 (t, 2H, J=11.7 Hz), 3.65 (tt, 1H, J=14.3, 4.5 Hz), 3.69 (s, 2H), 3.84 (t, 1H, J=9.3 Hz), 4.00 (m, 3H), 5.02 (m, 1H), 6.79 (d, 1H, J=8.1 Hz), 7.03 (m, 1H), 7.17 (d, 2H, J=8.4 Hz), 7.19 (m, 1H), 7.24 (d, 1H, J=6.9 Hz), 7.72 (d, 1H, J=8.4 Hz), 8.07 (d, 2H, J=8.4 Hz); ES-MS m/z 617 (M+H).

### EXAMPLE 306

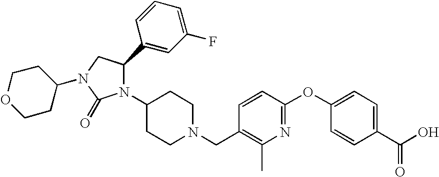

### COMPOUND 306: 4-(5-{4-[(R)-5-(3-Fluoro-phenyl)-2-oxo-3-(tetrahydro-pyran-4-yl)-imidazolidin-1-yl]-piperidin-1-ylmethyl}-6-methyl-pyridin-2-yloxy)-benzoic acid

COMPOUND 306 was isolated as a white solid (25 mg, 36% over 2 steps). ¹H NMR (CD₃OD) δ 1.60-1.90 (m, 5H), 1.95 (m, 2H), 2.41 (q, 1H, J=12.0 Hz), 2.50 (s, 3H), 3.10 (q, 2H, J=12.0 Hz), 3.23 (m, 1H), 3.50 (m, 4H), 3.63 (tt, 1H, J=12.0, 3.6 Hz), 3.85 (t, 1H, J=9.3 Hz), 3.98 (m, 3H), 4.30 (s, 2H), 4.80 (m, 1H), 6.94 (d, 1H, J=8.4 Hz), 7.14 (t, 1H, J=8.4 Hz), 7.21 (m, 1H), 7.22 (d, 2H, J=9.0 Hz), 7.28 (d, 1H, J=7.8 Hz), 7.47 (q, 1H, J=6.9 Hz), 7.86 (d, 1H, J=8.4 Hz), 8.10 (d, 2H, J=8.4 Hz); ES-MS m/z 589 (M+H).

### EXAMPLE 307

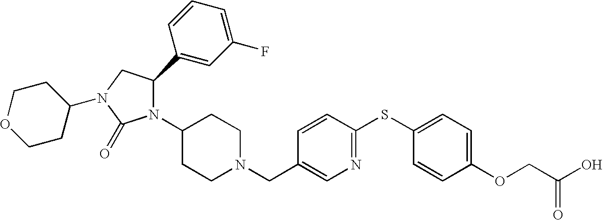

### COMPOUND 307: [4-(5-{4-[(R)-5-(3-Fluoro-phenyl)-2-oxo-3-(tetrahydro-pyran-4-yl)-imidazolidin-1-yl]-piperidin-1-ylmethyl}-pyridin-2-ylsulfanyl)-phenoxy]-acetic acid

COMPOUND 307 was isolated as a white solid (25 mg, 43% over 2 steps). ¹H NMR (CD₃OD) δ 1.36 (d, 2H, J=6.6 Hz), 1.60-1.95 (m, 6H), 2.29 (dq, 1H, J=12.0, 3.6 Hz), 2.73 (q, 2H, J=12.0 Hz), 3.18 (m, 1H), 3.25 (m, 1H), 3.51 (t, 2H, J=11.7 Hz), 3.57 (tt, 1H, J=12.0, 3.6 Hz), 3.83 (t, 1H, J=9.3 Hz), 3.94 (m, 1H), 4.00 (s, 2H), 4.01 (m, 2H), 4.58 (s, 2H), 4.78 (m, 1H), 6.85 (d, 1H, J=8.4 Hz), 7.06 (d, 2H, J=8.4 Hz), 7.13 (m, 1H), 7.18 (d, 1H, J=9.6 Hz), 7.25 (d, 1H, J=7.8 Hz), 7.43 (q, 1H, J=7.2 Hz), 7.51 (d, 2H, J=8.4 Hz), 7.59 (dd, 1H, J=8.4, 2.1 Hz), 8.35 (s, 1H); ES-MS m/z 621 (M+H).

### EXAMPLE 308

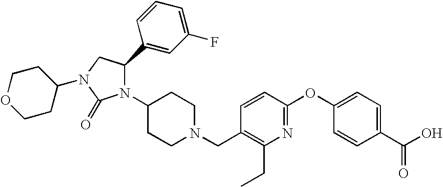

### COMPOUND 308: 4-(6-Ethyl-5-{4-[(R)-5-(3-fluoro-phenyl)-2-oxo-3-(tetrahydro-pyran-4-yl)-imidazolidin-1-yl]-piperidin-1-ylmethyl}-pyridin-2-yloxy)-benzoic acid

COMPOUND 308 was isolated as a white solid (16 mg, 21% over 2 steps). ¹H NMR (CD₃OD) δ 1.15 (t, 3H, J=6.9 Hz), 1.43 (dq, 1H, J=12.0, 3.6 Hz), 1.55-1.85 (m, 6H), 2.08 (dq, 1H, J=12.0, 3.6 Hz), 2.30 (q, 2H, J=12.0 Hz), 2.75 (q, 2H, J=7.5 Hz), 2.94 (d, 1H, J=8.7 Hz), 3.07 (d, 1H, J=10.8 Hz), 3.16 (m, 1H), 3.51 (t, 2H, J=11.6 Hz), 3.61 (tt, 1H, J=14.3, 4.5 Hz), 3.66 (s, 2H), 3.82 (t, 1H, J=9.3 Hz), 4.00 (m, 3H), 4.80 (m, 1H), 6.79 (d, 1H, J=8.1 Hz), 7.11 (dt, 1H, J=8.1, 2.7 Hz), 7.17 (m, 1H), 7.17 (d, 2H, J=8.4 Hz), 7.24 (d, 1H, J=8.1 Hz), 7.43 (q, 1H, J=9.2 Hz), 7.72 (d, 1H, J=8.4 Hz), 8.07 (d, 2H, J=8.4 Hz); ES-MS m/z 603 (M+H).

### EXAMPLE 309

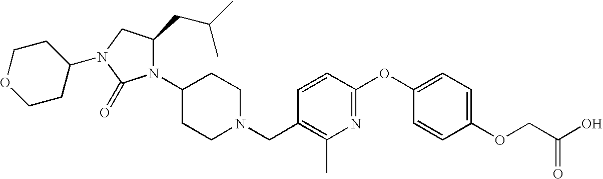

### COMPOUND 309: [4-(5-{4-[(R)-5-Isobutyl-2-oxo-3-(tetrahydro-pyran-4-yl)-imidazolidin-1-yl]-piperidin-1-ylmethyl}-6-methyl-pyridin-2-yloxy)-phenoxy]-acetic acid

COMPOUND 309 was isolated as a white solid (73 mg, 52% over 2 steps). ¹H NMR (CD₃OD) δ 1.00 (t, 6H, J=5.1 Hz), 1.45 (t, 1H, J=9.9 Hz), 1.55-2.00 (m, 8H), 2.20 (dq, 1H, J=12.0, 3.6 Hz), 2.40 (dq, 1H, J=12.0, 3.6 Hz), 2.53 (s, 3H), 2.91 (q, 2H, J=12.0 Hz), 3.05 (m, 1H), 3.38 (m, 5H), 3.60 (tt, 1H, J=12.0, 3.6 Hz), 3.74 (d, 1H, J=10.8 Hz), 3.87 (tt, 1H, J=13.2, 3.9 Hz), 4.02 (dd, 2H, J=11.7, 3.6 Hz), 4.13 (s, 2H), 4.49 (s, 2H), 6.67 (d, 1H, J=8.4 Hz), 7.00 (m, 4H), 7.78 (d, 1H, J=8.4 Hz); ES-MS m/z 581 (M+H). Anal. Calcd. for C₃₂H₄₄N₄O₆.0.4CH₂Cl₂: C, 63.3 1; H, 7.35; N, 9.11. Found: C, 63.38; H, 7.45; N, 9.00.

### EXAMPLE 310

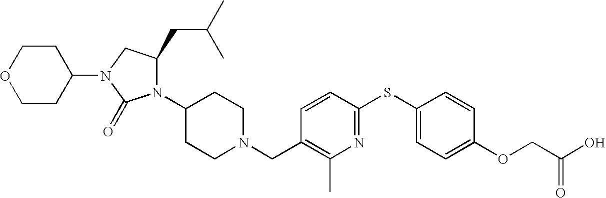

### COMPOUND 310: [4-(5-{4-[(R)-5-Isobutyl-2-oxo-3-(tetrahydro-pyran-4-yl)-imidazolidin-1-yl]-piperidin-1-ylmethyl}-6-methyl-pyridin-2-ylsulfanyl)-phenoxy]-acetic acid

COMPOUND 310 was isolated as a white powder (74.7 mg, 69% over 2 steps). ¹H NMR (CD₃OD) δ 0.97-1.02 (m, 6H), 1.40 (m, 1H), 1.54-1.82 (m, 6H), 1.89-1.99 (m, 2H), 2.20-2.32 (m, 1H), 2.40-2.52 (m, 1H), 2.62 (s, 3H), 3.02-3.14 (m, 3H), 3.44-3.80 (m, 9H), 3.99 (m, 2H), 4.28 (s, 2H), 4.63 (s, 1H), 4.66 (s, 1H), 6.68 (d, 1H, J=8.4 Hz), 7.08 (d, 2H, J=9.0 Hz), 7.53 (d, 2H, J=9.0 Hz), 7.61 (d, 1H, J=8.4 Hz); ¹³C NMR (CD₃OD) δ 22.2, 22.7, 25.0, 26.3, 28.2, 29.0, 31.2, 31.6, 45.2, 46.3, 50.7, 50.8, 53.6, 53.7, 57.9, 67.4, 68.6, 68.7, 117.9, 119.5, 121.5, 122.2, 138.9, 142.5, 160.3, 161.7, 161.9, 166.1, 174.1; ES-MS m/z 597 (M+H). Anal. Calcd. for C₃₂H₄₄N₄O₅S.0.9CH₂Cl₂: C, 58.70; H, 6.86; N, 8.32. Found: C, 58.89; H, 6.83; N, 8.07.

### EXAMPLE 311

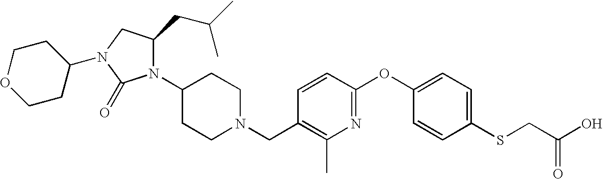

### COMPOUND 311: [4-(5-{4-[(R)-5-Isobutyl-2-oxo-3-(tetrahydro-pyran-4-yl)-imidazolidin-1-yl]-piperidin-1-ylmethyl}-6-methyl-pyridin-2-yloxy)-phenylsulfanyl]-acetic acid

COMPOUND 311 was isolated as a white solid (37 mg, 43% over 2 steps). ¹H NMR (CD₃OD) δ 1.01 (t, 6H, J=5.1 Hz), 1.45 (t, 1H, J=9.9 Hz), 1.55-1.85 (m, 6H), 2.00 (q, 2H, J=12.0 Hz), 2.37 (q, 1H, J=12.0 Hz), 2.54 (m, 1H), 2.55 (s, 3H), 3.06 (t, 1H, J=7.8 Hz), 3.24 (q, 2H, J=12.0 Hz), 3.48 (t, 2H, J=12.3 Hz), 3.59 (t, 1H, J=12.0 Hz), 3.66 (m, 2H), 3.73 (s, 2H), 3.74 (m, 1H), 3.83 (tt, 1H, J=12.0, 3.6 Hz), 4.02 (dd, 2H, J=11.7, 3.6 Hz), 4.39 (s, 2H), 6.88 (d, 1H, J=8.4 Hz), 7.12 (d, 2H, J=8.7 Hz), 7.52 (d, 2H, J=8.7 Hz), 7.88 (d, 1H, J=8.4 Hz); ES-MS m/z 597 (M+H).

### EXAMPLE 312

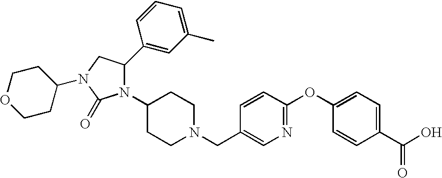

### COMPOUND 312: 4-(5-{4-[2-Oxo-3-(tetrahydro-pyran-4-yl)-5-m-tolyl-imidazolidin-1-yl]-piperidin-1-ylmethyl}-pyridin-2-yloxy)-benzoic acid

Following general procedure G, 3-piperidin-4-yl-1-(tetrahydro-pyran-4-yl)-4-m-tolyl-imidazolidin-2-one (105 mg, 0.32 mmol) was dissolved in CH₃CN (3 mL). Diisopropylethylamine (0.85 μL, 0.48 mmol) and 5-bromomethyl-pyridin-2-carbonitrile (102 mg, 0.35 mmol) were then added, and the reaction stirred at 60° C. for 18 h. Standard work-up and column chromatography on silica gel (NH₃Et₂O) gave the desired product, 4-(5-{4-[2-oxo-3-(tetrahydro-pyran-4-yl)-5-m-tolyl-imidazolidin-1-yl]-piperidin-1-ylmethyl}-pyridin-2-yloxy)-benzonitrile as a white solid (153 mg, 89%).

Following general procedure I, a portion of the above nitrile (110 mg, 0.20 mmol) was hydrolyzed using 10N NaOH in EtOH at 75° C. to afford COMPOUND 312 as a light beige powder (114 mg, 100%). ¹H NMR (CD₃OD) δ 1.45 (m, 1H), 1.50-1.75 (m, 6H), 2.12 (dq, 1H, J=10.8, 3.6 Hz), 2.25 (m, 2H), 2.33 (s, 3H), 2.88 (d, 1H, J=10.5 Hz), 3.05 (d, 1H, J=10.5 Hz), 3.12 (m, 1H), 3.47 (m, 3H), 3.63 (s, 2H), 3.74 (t, 1H, J=9.0 Hz), 3.96 (m, 3H), 4.68 (m, 1H), 6.98 (d, 1H, J=8.4 Hz), 7.15 (m, 51H), 7.23 (q, 1H, J=7.2 Hz), 7.80 (dd, 1H, J=8.4, 2.4 Hz), 8.06 (d, 2H, J=8.7 Hz); ES-MS m/z 571 (M+H).

### EXAMPLE 313

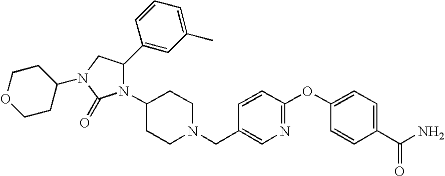

### COMPOUND 313: 4-(5-{4-[2-Oxo-3-(tetrahydro-pyran-4-yl)-5-m-tolyl-imidazolidin-1-yl]-piperidin-1-ylmethyl}-pyridin-2-yloxy)-benzamide

To a solution of 4-(5-{4-[2-oxo-3-(tetrahydro-pyran-4-yl)-5-m-tolyl-imidazolidin-1-yl]-piperidin-1-ylmethyl}-pyridin-2-yloxy)-benzonitrile (see EXAMPLE 312) (40 mg, 75 μmol) in TFA (1 mL) was added c. H₂SO₄ (2 drops). The reaction was heated to 100° C. for 16 h, and then concentrated to dryness under reduced pressure. The crude residue was then purified by column chromatography on silica gel (25:1, CH₂Cl₂/MeOH) to afford COMPOUND 313 as a white solid (25 mg, 61%). ¹H NMR (CDCl₃) δ 1.25 (m, 1H), 1.41 (d, 1H, J=11.7 Hz), 1.67 (m, 6H), 1.91 (m, 2H), 2.02 (m, 1H), 2.35 (s, 3H), 2.67 (d, 1H, J=10.2 Hz), 2.88 (d, 1H, J=10.2 Hz), 3.03 (m, 1H), 3.38 (s, 2H), 3.45 (m, 2H), 3.61 (t, 1H, J=9.0 Hz), 3.63 (m, 1H), 4.00 (m, 3H), 4.55 (m, 1H), 5.65 (s, 1H), 6.00 (s, 1H), 6.90 (d, 1H, J=8.4 Hz), 7.12 (m, 3H), 7.18 (m, 3H), 7.65 (m, 1H), 7.84 (d, 2H, J=7.2 Hz), 8.02 (s, 1H); ES-MS m/z 570 (M+H).

### EXAMPLE 314

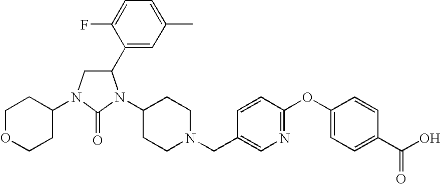

### COMPOUND 314: 4-(5-{4-[5-(2-Fluoro-5-methyl-phenyl)-2-oxo-3-(tetrahydro-pyran-4-yl)-imidazolidin-1-yl]-piperidin-1-ylmethyl}-pyridin-2-yloxy)-benzoic acid

Following general procedure G: a solution of 4-(2-fluoro-5-methyl-phenyl)-3-piperidin-4-yl-1-(tetrahydro-pyran-4-yl)-imidazolidin-2-one (406.5 mg, 1.126 mmol), 4-(5-bromomethyl-pyridin-2-yloxy)-benzonitrile (326.3 mg, 1.126 mmol) and DIPEA (195.8 μL, 1.126 mmol) in CH₃CN (6.0 mL) gave 4-(5-{4-[5-(2-fluoro-5-methyl-phenyl)-2-oxo-3-(tetrahydro-pyran-4-yl)-imidazolidin-1-yl]-piperidin-1-ylmethyl}-pyridin-2-yloxy)-benzonitrile (612.0 mg, 93%).

Following general procedure I, the above nitrile (308.3 mg, 0.530 mmol) gave COMPOUND 314 as a yellow powder (267 mg, 84%). ¹H NMR (CDCl₃) δ 1.46-1.78 (m, 6H), 1.79-1.93 (m, 1H), 2.29 (s, 3H), 2.33-2.80 (m, 2H), 3.20 (m, 2H), 3.46 (m, 3H), 3.71 (t, 1H, J=9 Hz), 3.91-4.05 (m, 6H), 4.89 (t, 1H, J=7.6 Hz), 6.88 (m, 1H), 7.00 (m, 2H), 7.13-7.16 (m, 3H), 8.01-8.08 (m, 1H), 8.04 (d, 3H, J=8.4 Hz), 8.28 (br s, 1H); ¹³C NMR (CDCl₃) δ 19.0, 24.2, 25.5, 28.0, 28.3, 45.1, 47.1, 47.6, 48.1, 49.6, 54.8, 65.3, 110.5, 114.0, 114.2, 119.0, 126.0, 126.9, 128.9, 129.9, 132.6, 141.2, 148.0, 155.0, 155.4, 157.7, 158.3, 161.9, 167.0; ES-MS m/z 589 (M+H). Anal. Calcd. for C₃₃H₃₇N₄O₅F.1.4CH₂Cl₂: C, 58.39; H, 5.67; N, 7.92. Found: C, 58.40; H, 5.91; N, 8.00.

### EXAMPLE 315

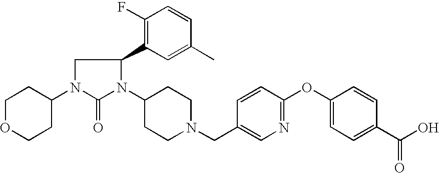

### COMPOUND 315: 4-(5-{4-[(R)-5-(2-Fluoro-5-methyl-phenyl)-2-oxo-3-(tetrahydro-pyran-4-yl)-imidazolidin-1-yl]-piperidin-1-ylmethyl}-pyridin-2-yloxy)-benzoic acid

Using general procedure A: to a stirred solution of 4-(2-fluoro-5-methyl-phenyl)-3-piperidin-4-yl-1-(tetrahydro-pyran-4-yl)-imidazolidin-2-one (1.7 g, 4.89 mmol) in CH₂Cl₂ (40 mL) at room temperature were added 4-(5-formyl-pyridine-2-yloxy)-benzoic acid tert-butyl ester (1.6 g, 5.38 mmol), glacial AcOH (10 drops) and sodium triacetoxyborohydride (1.55 g, 7.3 mmol) and the resultant solution was stirred at room temperature overnight to afford 4-(5-{4-[5-(2-fluoro-5-methyl-phenyl)-2-oxo-3-(tetrahydro-pyran-4-yl)-imidazolidin-1-yl]-piperidin-1-ylmethyl}-pyridine-2-yloxy)-benzoic acid tert-butyl ester as white foam solid (2.76 g, 87.6%).

A solution of the above tert-butyl ester (2.76 g, 4.3 mmol) and 6N HCl (30 mL) was stirred at room temperature for 3 hrs. The reaction mixture was cooled in an ice-water bath and neutralized with a 10N NaOH solution to pH=3.0-4.0 and extracted with 10% MeOH/CH₂Cl₂ (50 mL×2). The organic layer was dried with Na₂SO₄, filtered and concentrated to dryness to give COMPOUND 315 as a white foam solid (2.45 g, 100%). ¹H NMR (CD₃OD) δ 1.48 (m, 1H), 1.64-1.79 (m, 6H), 1.81-1.83 (m, 1H), 2.34 (m, 5H), 3.23-3.26 (m, 3H), 3.38 (m, 1H), 3.47-4.03 (m, 9H), 5.02-50.3 (m, 1H), 7.03-7.06 (m, 2H), 7.16-7.26 (m, 4H), 7.5 (dd, 1H, J=6.0, 3.0 Hz), 8.07-8.12 (m, 3H); 1 ¹³C NMR (CD₃OD) δ 19.88, 27.49, 28.87, 29.86, 30.21, 47.26, 47.55, 47.83, 49.53, 50.72, 50.98, 52.09, 52.3, 53.98, 57.50, 67.20, 67.29, 112.10, 115.75, 116.03, 120.38, 124.68, 128.39, 128.54, 129.22, 130.54, 130.79, 130.89, 131.59, 134.73, 142.88, 149.65, 157.50, 157.63, 160.42, 160.73, 163.69. Anal. Calcd. for C₃₃H₃₇N₄O₅F.0.55CH₂Cl₂: C, 63.42; H, 6.04; N, 8.82. Found: C, 63.34; H, 6.19; N, 9.01.

### EXAMPLE 316

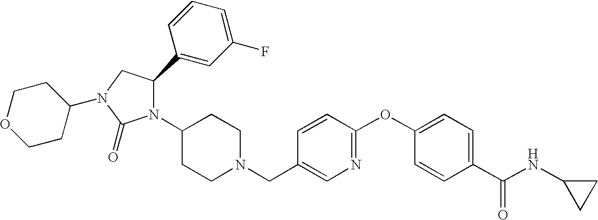

### COMPOUND 316: N-Cyclopropyl-4-(5-{4-[(R)-5-(3-fluoro-phenyl)-2-oxo-3-(tetrahydro-pyran-4-yl)-imidazolidin-1-yl]-piperidin-1-ylmethyl}-pyridin-2-yloxy)-benzamide

Following general procedure A: (R)-4-(3-Fluoro-phenyl)-3-piperidin-4-y-1-1-(tetrahydro-pyran-4-yl)-imidazolidin-2-one (42 mg, 0.12 mmol) and N-cyclopropyl-4-(5-formyl-pyridin-2-yloxy)-benzamide (44 mg, 0.16 mmol) were combined in CH₂Cl₂ (1.5 mL) and treated with sodium triacetoxyborohydride (60 mg, 0.28 mmol) at room temperature for 16 h. After standard work-up, the crude material was purified by flash column chromatography on silica gel (50:1 EtOAc/MeOH) to afford COMPOUND 316 as a white solid (58 mg, 78%). ¹H NMR (CDCl₃) δ 0.61 (m, 2H), 0.87 (q, 2H, J=6.6 Hz), 1.21 (dq, 1H, J=12.0, 3.6 Hz), 1.44 (d, 1H, J=12.0 Hz), 1.64 (m, 5H), 1.75 (m, 3H), 1.99 (m, 1H), 2.69 (d, 1H, J=10.5 Hz), 2.89 (m, 2H), 3.03 (m, 1H), 3.38 (s, 2H), 3.46 (m, 2H), 3.66 (t, 2H, J=9.0 Hz), 3.99 (m, 3H), 4.58 (m, 1H), 6.17 (s, 1H), 6.87 (d, 1H, J=8.4 Hz), 6.95-7.13 (m, 3H), 7.14 (d, 2H, J=8.7 Hz), 7.31 (q, 1H, J=7.5 Hz), 7.62 (d, 1H, J=6.9 Hz), 7.76 (d, 2H, J=8.4 Hz), 8.00 (s, 1H); ES-MS m/z 614 (M+H).

### EXAMPLE 317

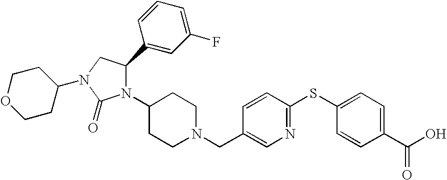

### COMPOUND 317: 4-(5-{4-[(R)-5-(3-Fluoro-phenyl)-2-oxo-3-(tetrahydro-pyran-4-yl)-imidazolidin-1-yl]-piperidine-1-ylmethyl}-pyridin-2-ylsulfanyl)-benzoic acid

Following general procedure G, (R)-4-(3-fluoro-phenyl)-3-piperidin-4-yl-1-(tetrahydro-pyran-4-yl)-imidazolidin-2-one (54 mg, 0.16 mmol) was dissolved in CH₃CN (2 mL). Diisopropylethylamine (35 μL, 0.20 mmol) and 4-(5-bromomethyl-pyridin-2-ylsulfanyl)-benzoic acid methyl ester (47 mg, 0.14 mmol) were then added, and the reaction stirred at 40° C. for 18 h. Standard work-up and purification afforded 4-(5-{4-[(R)-5-(3-fluoro-phenyl)-2-oxo-3-(tetrahydro-pyran-4-yl)-imidazolidin-1-yl]-piperidine-1-ylmethyl}-pyridin-2-ylsulfanyl)-benzoic acid methyl ester (70 mg, 84%).

Following general procedure H, the above ester (68 mg, 0.11 mmol) afforded COMPOUND 317 as a white solid (50 mg, 76%). ¹H NMR (CD₃OD) δ 1.54 (dq, 1H, J=12.6, 3.9 Hz), 1.65 (m, 4H), 1.78 (d, 2H, J=12.0 Hz), 2.15 (dq, 1H, J=12.3, 3.6 Hz), 2.42 (q, 2H, J=12.0 Hz), 3.05 (d, 1H, J=11.7 Hz), 3.15 (m, 2H), 3.51 (t, 2H, J=11.5 Hz), 3.62 (tt, 1H, J=12.6, 3.6 Hz), 3.78 (s, 2H), 3.82 (t, 1H, J=9.3 Hz), 3.97 (m, 3H), 4.79 (m, 1H), 7.06 (d, 1H, J=7.5 Hz), 7.16 (d, 1H, J=9.6 Hz), 7.23 (d, 1H, J=7.5 Hz), 7.40 (q, 1H, J=6.9 Hz), 7.61 (d, 2H, J=8.1 Hz), 7.66 (dd, 1H, J=8.4, 2.4 Hz), 8.06 (d, 2H, J=8.1 Hz), 8.37 (d, 1H, J=1.5 Hz); ES-MS m/z 591 (M+H).

### EXAMPLE 318

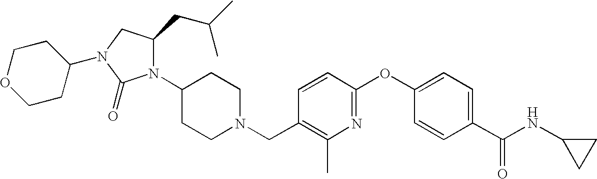

### COMPOUND 318: N-Cyclopropyl-4-(5-{4-[(R)-5-isobutyl-2-oxo-3-(tetrahydro-pyran-4-yl)-imidazolidin-1-yl]-1-piperidin-1-ylmethyl}-6-methyl-pyridin-2-yloxy)-benzamide

Following general procedure A: (R)-4-isobutyl-3-piperidin-4-yl-1-(tetrahydro-pyran-4-yl)-imidazolidin-2-one (63 mg, 0.20 mmol) and N-cyclopropyl-4-(5-formyl-6-methyl-pyridin-2-yloxy)-benzamide (79 mg, 0.26 mmol) were combined in CH₂Cl₂ (2 mL) and treated with sodium triacetoxyborohydride (86 mg, 0.41 mmol) at room temperature for 16 h. After standard work-up, the crude material was purified by flash column chromatography on silica gel (50:1, EtOAc/MeOH) to afford COMPOUND 318 as a white solid (37 mg, 31%). ¹H NMR (CDCl₃) δ 0.62 (m, 2H), 0.87 (q, 2H, J=6.6 Hz), 0.95 (t, 6H, J=6.0 Hz), 1.41 (m, 1H), 1.55-1.65 (m, 6H), 1.75 (m, 3H), 1.95 (dq, 1H, J=12.3, 3.6 Hz), 2.07 (m, 2H), 2.45 (s, 3H), 2.90 (m, 4H), 3.35 (t, 1H, J=8.4 Hz), 3.41 (s, 2H), 3.50 (t, 2H, J=9.0 Hz), 3.60 (m, 2H), 3.99 (m, 3H), 6.19 (s, 1H), 6.65 (d, 1H, J=8.4 Hz), 7.13 (d, 2H, J=8.7 Hz), 7.60 (d, 1H, J=8.1 Hz), 7.73 (d, 2H, J=8.7 Hz); ¹³C NMR (CDCl₃) δ 7.04 (2C), 21.88, 22.25, 23.54, 24.64, 25.25, 29.57, 30.10, 30.60, 31.95, 44.67, 44.98, 48.97, 51.40, 52.09, 53.78, 53.88, 59.39, 67.60, 67.71, 108.87, 120.25 (2C), 128.15, 129.06 (2C), 130.33, 141.40, 156.98, 158.04, 160.35, 161.21, 168.72; ES-MS m/z 590 (M+H). Anal. Calcd. for C₃₄H₄₇N₅O₄.0.2CH₂Cl₂: C, 67.70; H, 7.87; N, 11.54. Found: C, 67.97; H, 8.03; N, 11.54.

### EXAMPLE 319

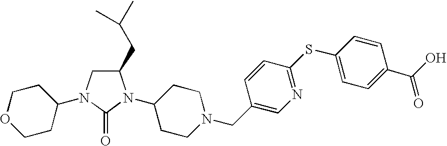

### COMPOUND 319: 4-(5-{4-[(R)-5-Isobutyl-2-oxo-3-(tetrahydro-pyran-4-yl)-imidazolidin-1-yl]-piperidin-1-ylmethyl}-pyridin-2-ylsulfanyl)-benzoic acid

Following general procedure G: a solution of (R)-4-isobutyl-3-piperidin-4-yl-1-(tetrahydro-pyran-4-yl)-imidazolidin-2-one (110 mg, 0.355 mmol), 4-(5-bromomethyl-pyridin-2-ylsulfanyl)-benzoic acid methyl ester (120 mg, 0.355 mmol) and DIPEA (0.1 mL, 0.54 mmol) in CH₃CN (4 mL) was heated to 50° C. overnight. Standard work-up and purification afforded 4-(5-{4-[(R)-5-isobutyl-2-oxo-3-(tetrahydro-pyran-4-yl)-imidazolidin-1-yl]-piperidin-1-ylmethyl}-pyridin-2-ylsulfanyl)-benzoic acid methyl ester. Following general procedure H, the methyl ester afforded COMPOUND 319 as a white solid (110 mg, 56% over 2 steps). ¹H NMR (CDCl₃) δ 0.88 (d, 6H, J=6.3 Hz), 1.31-1.38 (m, 1H), 1.49-1.95 (m, 6H), 2.34-2.43 (m, 1H), 2.51-2.61 (m, 1H), 2.78-2.92 (m, 3H), 3.34-3.65 (m, 6H), 3.89-4.10 (m, 6H), 6.93 (d, 1H, J=8.1 Hz), 7.59 (d, 2H, J=8.1 Hz), 8.00-8.05 (m, 3H), 8.40 (br s, 1H); ¹³C NMR (CDCl₃) δ 21.23, 24.14, 24.90, 25.37, 29.67, 30.24, 43.84, 44.65, 48.63, 48.79, 51.13, 51.87, 56.89, 67.12, 67.24, 77.27, 121.85, 122.34, 130.95, 132.73, 134.53, 135.01, 139.86, 151.15, 159.83, 162.70, 168.95; ES-MS m/z 553 (M+1). Anal. Calcd. for C₃₀H₄₀N₄O₄S.0.92CH₂Cl₂.0.56H₂O: C, 57.94; H, 6.75; N, 8.74. Found: C, 57.96; H, 6.78; N, 8.65.

Examples 320 to 326 were prepared following the scheme illustrated below. RNH₂ is as defined in the table and Y and Z are as defined in the individual examples.

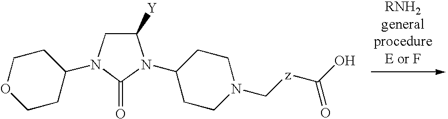

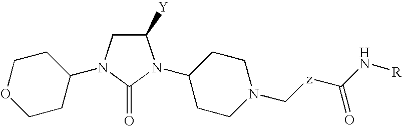

| Example | RNH₂ |
|---|---|
| 320 | Isopropylamine |
| 321 | Cyclopropylamine |
| 322 | Cyclopropylamine |
| 323 | Methoxylamine hydrochloride |
| 324 | Isopropylamine |
| 325 | methylamine hydrochloride |
| 326 | methylamine hydrochloride |

### EXAMPLE 320

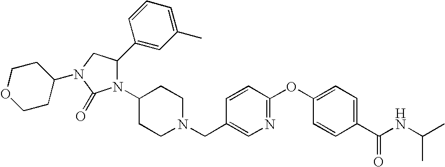

### COMPOUND 320: N-Isopropyl-4-(5-{4-[2-oxo-3-(tetrahydro-pyran-4-yl)-5-m-tolyl-imidazolidin-1-yl]-piperidin-1-ylmethyl}-pyridin-2-yloxy)-benzamide

Following general procedure F: 4-(5-{4-[2-Oxo-3-(tetrahydro-pyran-4-yl)-5-m-tolyl-imidazolidin-1-yl]-piperidin-1-ylmethyl}-pyridin-2-yloxy)-benzoic acid (COMPOUND 312) afforded COMPOUND 320 as white solid (13 mg, 38%). ¹H NMR (CDCl₃) δ 1.25 (m, 1H), 1.26 (d, 6H, J=6.9 Hz), 1.42 (m, 1H), 1.66 (m, 5H), 1.91 (m, 2H), 2.00 (m, 1H), 2.35 (s, 3H), 2.67 (d, 1H, J=10.8 Hz), 2.88 (m, 1H, J=10.8 Hz), 3.03 (m, 1H), 3.37 (s, 2H), 3.47 (m, 2H), 3.61 (t, 1H, J=9.0 Hz), 3.63 (m, 1H), 4.00 (m, 3H), 4.27 (sept, 1H, J=7.5 Hz), 4.55 (m, 1H), 5.81 (d, 1H, J=7.8 Hz), 6.87 (d, 1H, J=8.4 Hz), 7.14 (m, 5H), 7.22 (d, 1H, J=9.0 Hz), 7.63 (d, 1H, J=7.5 Hz), 7.77 (d, 2H, J=7.2 Hz), 8.00 (s, 1H); ES-MS m/z 612 (M+H).

### EXAMPLE 321

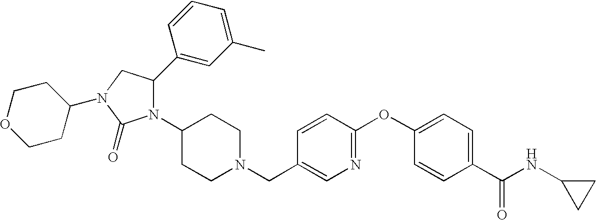

### COMPOUND 321: N-Cyclopropyl-4-(5-{4-[2-oxo-3-(tetrahydro-pyran-4-yl)-5-m-tolyl-imidazolidin-1-yl]-piperidin-1-ylmethyl}-pyridin-2-yloxy)-benzamide

Following general procedure F: 4-(5-{4-[2-oxo-3-(tetrahydro-pyran-4-yl)-5-m-tolyl-imidazolidin-1-yl]-piperidin-1-ylmethyl}-pyridin-2-yloxy)-benzoic acid (COMPOUND 312) afforded COMPOUND 321 as white solid (16 mg, 47%). ¹H NMR (CDCl₃) δ 0.61 (s, 2H), 0.88 (m, 2H), 1.25 (m, 1H), 1.42 (m, 1H), 1.66 (m, 5H), 1.90 (m, 2H), 2.00 (m, 1H), 2.34 (s, 3H), 2.67 (d, 1H, J=10.8 Hz), 2.88 (m, 2H), 3.04 (m, 1H), 3.37 (s, 2H), 3.47 (m, 2H), 3.61 (t, 1H, J=9.0 Hz), 3.63 (m, 1H), 4.00 (m, 3H), 4.55 (m, 1H), 6.17 (s, 1H), 6.87 (d, 1H, J=8.4 Hz), 7.12 (m, 5H), 7.21 (d, 1H, J=7.5 Hz), 7.62 (d, 1H, J=7.5 Hz), 7.75 (d, 2H, J=7.5 Hz), 8.00 (s, 1H); ES-MS m/z 610 (M+H).

### EXAMPLE 322

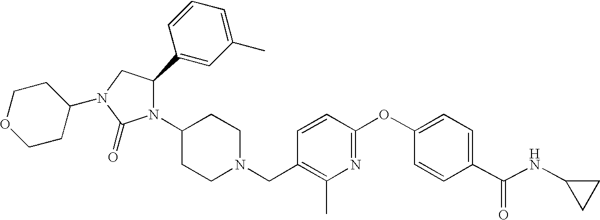

### COMPOUND 322: N-Cyclopropyl-4-(6-methyl-5-{4-[(R)-2-oxo-3-(tetrahydro-pyran-4-yl)-5-m-tolyl-imidazolidin-1-yl]-piperidin-1-ylmethyl}-pyridin-2-yloxy)-benzamide

Following general procedure F: 4-(6-Methyl-5-{4-[(R)-2-oxo-3-(tetrahydro-pyran-4-yl)-5-m-tolyl-imidazolidin-1-yl]-piperidine-1-ylmethyl}-pyridin-2-yloxy)-benzoic acid (COMPOUND 278) afforded COMPOUND 322 as white solid (9 mg, 22%). ¹H NMR (CDCl₃) δ 0.62 (m, 2H), 0.87 (m, 2H), 1.24 (m, 1H), 1.41 (m, 1H), 1.65 (m, 5H), 1.91 (m, 2H), 1.98 (q, 1H, J=11.1 Hz), 2.35 (s, 3H), 2.38 (s, 3H), 2.64 (d, 1H, J=11.4 Hz), 2.83 (d, 1H, J=11.4 Hz), 2.90 (sept, 1H, J=3.6 Hz), 3.05 (m, 1H), 3.32 (s, 2H), 3.55 (m, 2H), 3.76 (t, 1H, J=9.0 Hz), 3.77 (m, 1H), 3.99 (m, 3H), 4.55 (m, 1H), 6.18 (s, 1H), 6.60 (d, 1H, J=8.1 Hz), 7.11 (m, 5H), 7.22 (d, 1H, J=7.5 Hz), 7.50 (d, 1H, J=8.4 Hz), 7.72 (d, 2H, J=8.7 Hz); ES-MS m/z 624 (M+H).

### EXAMPLE 323

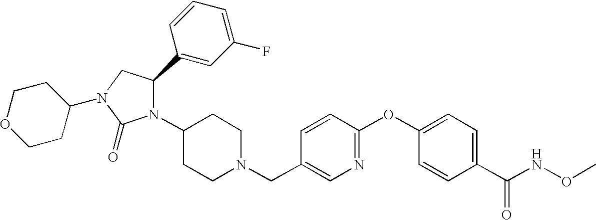

### COMPOUND 323: 4-(5-{4-[(R)-5-(3-Fluoro-phenyl)-2-oxo-3-(tetrahydro-pyran-4-yl)-imidazolidin-1-yl]-piperidine-1-ylmethyl}-pyridin-2-yloxy)-N-methoxy-benzamide

Following general procedure E: 4-(5-{4-[(R)-5-(3-fluoro-phenyl)-2-oxo-3-(tetrahydro-pyran-4-yl)-imidazolidin-1-yl]-piperidine-1-ylmethyl}-pyridin-2-yloxy)-benzoic acid (COMPOUND 288) afforded COMPOUND 323 as white solid (14 mg, 43%). ¹H NMR (CDCl₃) δ 1.19 (dq, 1H, J=12.0, 3.6 Hz), 1.42 (d, 1H, J=12.0 Hz), 1.65 (m, 5H), 1.80-2.05 (m, 3H), 2.68 (d, 1H, J=11.1 Hz), 2.85 (d, 1H, J=10.5 Hz), 3.03 (m, 1H), 3.37 (s, 2H), 3.45 (m, 2H), 3.64 (t, 1H, J=9.0 Hz), 3.65 (m, 1H), 3.89 (s, 3H), 4.02 (m, 3H), 4.58 (m, 1H), 6.89 (d, 1H, J=8.4 Hz), 7.03 (m, 2H), 7.10 (d, 1H, J=7.5 Hz), 7.16 (d, 2H, J=8.7 Hz), 7.31 (q, 1H, J=7.2 Hz), 7.63 (dd, 1H, J=8.4, 1.2 Hz), 7.77 (d, 2H, J=8.4 Hz), 8.00 (d, 1H, J=1.8 Hz), 8.75 (s, 1H); ES-MS m/z 604 (M+H).

### EXAMPLE 324

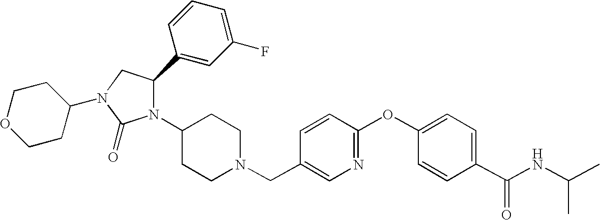

### COMPOUND 324: 4-(5-{4-[(R)-5-(3-Fluoro-phenyl)-2-oxo-3-(tetrahydro-pyran-4-yl)-imidazolidin-1-yl]-piperidin-1-ylmethyl}-pyridin-2-yloxy)-N-isopropyl-benzamide

Following general procedure E: 4-(5-{4-[(R)-5-(3-fluoro-phenyl)-2-oxo-3-(tetrahydro-pyran-4-yl)-imidazolidin-1-yl]-piperidine-1-ylmethyl}-pyridin-2-yloxy)-benzoic acid (COMPOUND 288) afforded COMPOUND 324 as white solid (29 mg, 84%). ¹H NMR (CDCl₃) δ 1.19 (dq, 1H, J=12.0, 3.6 Hz), 1.26 (d, 6H, J=6.3 Hz), 1.44 (d, 1H, J=12.0 Hz), 1.65 (m, 5H), 1.80-2.05 (m, 3H), 2.68 (d, 1H, J=10.5 Hz), 2.85 (d, 1H, J=9.3 Hz), 3.03 (m, 1H), 3.37 (s, 2H), 3.45 (m, 2H), 3.66 (t, 1H, J=9.0 Hz), 3.67 (m, 1H), 4.02 (m, 3H), 4.28 (m, 1H), 4.58 (m, 1H), 5.84 (d, 1H, J=8.1 Hz), 6.87 (d, 1H, J=8.4 Hz), 7.03 (m, 2H), 7.10 (d, 1H, J=7.5 Hz), 7.14 (d, 2H, J=8.7 Hz), 7.31 (q, 1H, J=7.2 Hz), 7.62 (dd, 1H, J=8.4, 2.1 Hz), 7.77 (d, 2H, J=8.7 Hz), 8.00 (s, 1H); ES-MS m/z 616 (M+H).

### EXAMPLE 325

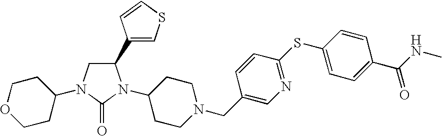

### COMPOUND 325: N-Methyl-4-(5-{4-[(R)-2-oxo-3-(tetrahydro-pyran-4-yl)-5-thiophen-3-yl-imidazolidin-1-yl]-piperidin-1-ylmethyl}-pyridin-2-ylsulfanyl)-benzamide

Following general procedure E: 4-(5-{4-[(R)-2-oxo-3-(tetrahydro-pyran-4-yl)-5-thiophen-3-yl-imidazolidin-1-yl]-piperidin-1-ylmethyl}-pyridin-2-ylsulfanyl)-benzoic acid (COMPOUND 292) afforded COMPOUND 325 as a white foam (56 mg, 92%). ¹H NMR (CDCl₃) δ 1.16-1.25 (m, 1H), 1.40-1.44 (m, 1H), 1.64-1.70 (m, 5H), 1.82-2.01 (m, 3H), 2.65-2.69 (m, 1H), 2.81-2.84 (m, 1H), 3.03 (d, 3H, J=4.8 Hz), 3.09 (dd, 1H, J=8.1, 6.3 Hz), 3.36 (s, 2H), 3.44-3.67 (m, 4H), 3.98-4.02 (m, 3H), 4.72 (dd, 1H, J=8.7, 6.3 Hz), 6.14 (br s, 1H), 6.98 (d, 1H, J=8.4 Hz), 7.04-7.06 (m, 1H), 7.18-7.19 (m, 1H), 7.31 (dd, 1H, J=4.8, 3.0 Hz), 7.43 (dd, 1H, J=8.4, 2.1 Hz), 7.57 (d, 2H, J=8.1 Hz), 7.75 (d, 2H, J=8.4 Hz), 8.30 (d, 1H, J=1.8 Hz); ¹³C NMR (CDCl₃) δ 28.94, 29.87, 30.10, 30.36, 48.47, 48.71, 51.65, 51.81, 52.99, 53.16, 59.34, 67.18, 67.25, 77.28, 122.26, 122.51, 125.73, 127.07, 127.92, 131.28, 133.41, 134.51, 136.04, 137.76, 143.45, 150.28, 157.78, 159.64, 167.52; ES-MS m/z 614 (M+Na). Anal. Calcd. for C₃₁H₃₇N₅O₃S₂.1.3H₂O: C, 60.52; H, 6.49; N, 11.38. Found: C, 60.15; H, 6.41; N, 11.72.

### EXAMPLE 326

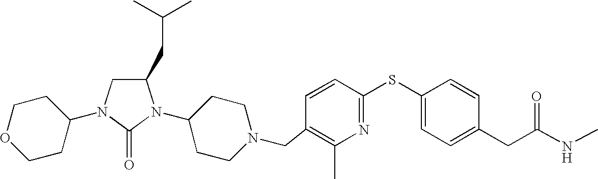

### COMPOUND 326: 2-[4-(5-{4-[(R)-5-Isobutyl-2-oxo-3-(tetrahydro-pyran-4-yl)-imidazolidin-1-yl]-piperidin-1-ylmethyl}-6-methyl-pyridin-2-ylsulfanyl)-phenyl-N-methyl-acetamide

Following general procedure E: [4-(5-{4-[(R)-5-isobutyl-2-oxo-3-(tetrahydro-pyran-4-yl)-imidazolidin-1-yl]-piperidin-1-ylmethyl}-6-methyl-pyridin-2-ylsulfanyl)-phenyl]-acetic acid (COMPOUND 296) gave COMPOUND 326 as a white foam (40 mg, 90%). ¹H NMR (CDCl₃) δ 0.94 (t, 6H, J=6.3 Hz), 1.61-1.76 (m, 10H), 2.01-2.11 (m, 3H), 2.53 (s, 3H), 2.80 (d, 3H, J=4.8 Hz), 2.85-2.90 (m, 3H), 3.32-3.50 (m, 6H), 3.57-3.60 (m, 4H), 3.91-4.02 (m, 3H), 6.72 (d, 1H, J=8.1 Hz), 7.30 (d, 2H, J=8.1 Hz), 7.38 (d, 1H, J=8.4 Hz), 7.56 (d, 2H, J=8.1 Hz).

### EXAMPLE 327

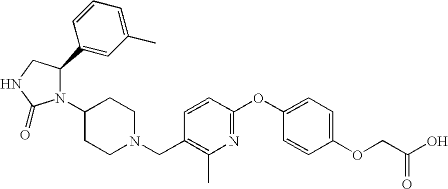

### COMPOUND 327: (4-{6-Methyl-5-[4-((R)-2-oxo-5-m-tolyl-imidazolidin-1-yl)-piperidin-1-ylmethyl]-pyridin-2-yloxy}-phenoxy)-acetic acid

To a cooled solution (0° C.) of [(R)-1-(3-methyl-phenyl)-2-hydroxy-ethyl]-carbamic acid tert-butyl ester (0.90 g, 3.9 mmol) and triethylamine (0.70 mL, 5.1 mmol) in CH₂Cl₂ (13 mL) was added methanesulfonyl chloride (0.33 mL, 4.3 mmol) and the solution stirred for 3 hours while warming to room temperature. Standard work-up afforded the desired mesylated intermediate (1.44 g, 87%). This material was then dissolved in DMF (8 mL) and potassium phthalimide added (0.87 g, 4.7 mmol). The reaction was heated to 100° C. for 16 h. The thick suspension was concentrated under reduced pressure and dried in vacuo. Aqueous work-up and purification afforded [(R)-2-(1,3-dioxo-1,3-dihydro-isoindol-2-yl)-1-m-tolyl-ethyl]-carbamic acid tert-butyl ester as a white solid (0.68 g, 50%).

Following general procedure C, the above compound (0.68 g, 1.9 mmol) was treated with TFA (1 mL) in CH₂Cl₂ (5 mL) for ˜4 h to give 2-(2-amino-2-m-tolyl-ethyl)-isoindole-1,3-dione (0.41 g, 83%). The crude amine and N-Boc-4-piperidone (0.33 g, 1.6 mmol) were then reacted according to general procedure A to afford, after standard work-up and column chromatography with silica gel (50:1:0.1, CH₂Cl₂/MeOH/NH₄OH), 4-[(R)-2-(1,3-dioxo-1,3-dihydro-isoindol-2-yl)-1-m-tolyl-ethylamino]-piperidine-1-carboxylic acid tert-butyl ester as a white solid (0.34 g, 49%).

The compound above (0.34 g, 0.76 mmol) was dissolved in ethanol (3 mL) and treated with hydrazine hydrate (0.37 mL, 7.6 mmol) for 16 h at room temperature. Standard work-up gave the crude amine (0.25 g, 100%). This material was then dissolved in DMF (1.5 mL) and treated with 1,1-carbonyldiimidazole (135 mg, 0.84 mmol) for 1 h. Standard work-up afforded 4-((R)-2-oxo-5-m-tolyl-imidazolidin-1-yl)-piperidine-1-carboxylic acid tert-butyl ester (0.31 g, excess). Following general procedure C, this crude intermediate afforded (R)-1-piperidin-4-yl-5-m-tolyl-imidazolidin-2-one (131 mg, 69% over 3 steps). ¹H NMR (CDCl₃) δ 1.14 (dq, 1H, J=12.0, 3.6 Hz), 1.50 (d, 1H, J=12.0 Hz), 1.70 (d, 1H, J=12.0 Hz), 1.82 (dq, 1H, J=12.0, 3.6 Hz), 2.35 (s, 3H), 2.53 (dt, 1H, J=9.0, 2.7 Hz), 2.63 (dt, 1H, J=9.0, 2.7 Hz), 2.92 (d, 1H, J=12.0 Hz), 3.08 (d, 1H, J=12.0 Hz), 3.20 (m, 1H), 3.66 (tt, 1H, J=12.3, 2.8 Hz), 3.73 (t, 1H, J=9.0 Hz), 4.49 (s, 1H), 4.71 (m, 1H), 7.14 (m, 2H), 7.17 (s, 1H), 7.23 (d, 1H, J=7.5 Hz).

Following general procedure A, (R)-1-piperidin-4-yl-5-m-tolyl-imidazolidin-2-one (40 mg, 0.15 mmol) and [4-(5-formyl-6-methyl-pyridin-2-yloxy)-phenoxy]-acetic acid tert-butyl ester (69 mg, 0.20 mmol) were combined in CH₂Cl₂ (1.5 mL) and treated with sodium triacetoxyborohydride (64 mg, 0.30 mmol) at room temperature for 16 h. After standard work-up, the crude material was purified by flash column chromatography on silica gel (50:1, EtOAc/MeOH) to afford (R)-(4-{6-methyl-5-[4-(2-oxo-5-m-tolyl-imidazolidin-1-yl)-piperidin-1-ylmethyl]-pyridin-2-yloxy}-phenoxy)-acetic acid tert-butyl ester as a white solid (56 mg, 63%).

Following general procedure C, the above ester (53 mg, 90 μmol) was dissolved in CH₂Cl₂ (1 mL) and treated with TFA (0.5 mL), stirring for 16 h. Standard work-up and purification by column chromatography on silica gel (10:1:0.1, CH₂Cl₂/MeOH/NH₄OH) gave COMPOUND 327 as a white solid (40 mg, 83%). ¹H NMR (CD₃OD) δ 1.89 (m, 3H), 2.39 (s, 3H), 2.43 (m, 1H), 2.48 (s, 3H), 3.09 (m, 2H), 3.50 (m, 5H), 3.77 (t, 1H, J=8.7 Hz), 4.28 (s, 2H), 4.72 (s, 2H), 6.75 (d, 1H, J=8.7 Hz), 7.04 (m, 4H), 7.24 (m, 4H), 7.77 (d, 1H, J=8.7 Hz); ¹³C NMR (CD₃OD) δ 20.48, 21.01, 26.80, 27.50, 48.10, 49.49, 52.13 (2C), 56.61, 59.69, 65.32, 108.38, 115.86 (2C), 118.15, 122.33 (2C), 124.16, 127.70, 129.13, 129.37, 139.13, 141.97, 144.30, 148.11, 155.84, 158.35, 163.43, 164.59, 171.63; ES-MS m/z 531 (M+H).

### EXAMPLE 328

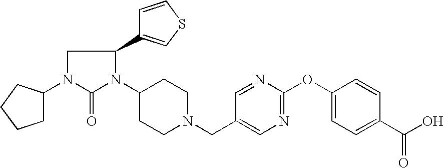

### COMPOUND 328: 4-{5-[4-((R)-3-Cyclopentyl-2-oxo-5-thiophen-3-yl-imidazolidin-1-yl)-piperidin-1-ylmethyl]-pyrimidin-2-yloxy}-benzoic acid

To a solution of ((R)-2-hydroxy-1-thiophen-3-yl-ethyl)-carbamic acid tert-butyl ester (700 mg, 2.88 mmol), phthalimide (465 mg, 3.16 mmol), triphenyl phosphine (896 mg, 2.96 mmol) in dry THF (10 mL) at 0° C. was added diethyl azodicarboxylate (0.49 mL, 3.2 mmol) dropwise. The mixture was stirred at room temperature for 2 h, then concentrated. The residue was dissolved in EtOH (20 mL) and hydrazine hydrate (1.8 mL, 1.8 mmol) was added. The mixture was stirred at room temperature overnight, filtered and evaporated to dryness. Aqueous work-up and purification afforded ((R)-2-amino-1-thiophen-3-yl-ethyl)-carbamic acid tert-butyl ester as a colorless oil (230 mg, 38%).

Using general procedure A, the above amine (230 mg, 0.95 mmol), cyclopentanone (84 μL, 0.95 mmol), sodium triacetoxyborohydride (297 mg, 1.33 mmol) and acetic acid (3 μL) in CH₂Cl₂ (15 mL) gave crude ((R)-2-cyclopentylamino-1-thiophen-3-yl-ethyl)-carbamic acid tert-butyl ester as a colorless oil (264 mg, 89%).

Using general procedure C, the above substrate (264 mg, 0.850 mmol) and TFA (2 mL) in CH₂Cl₂ (4 mL) gave crude (R)-N²-cyclopentyl-1-thiophen-3-yl-ethane-1,2-diamine as a colorless oil (157 mg, 88%).

Using general procedure A, the above amine (149 mg, 0.749 mmol), glacial AcOH (2 μL, 0.03 mmol) and NaBH(OAc)₃ (223 mg, 1.05 mmol) in CH₂Cl₂ (10 mL) gave crude 4-((R)-2-cyclopentylamino-1-thiophen-3-yl-ethylamino)-piperidine-1-carboxylic as a white foam (246 mg, 84%).

Following general procedure K: to the solution of the above diamine (246 mg, 0.630 mmol) in CH₂Cl₂ (8 ml) and pyridine (110 uL, 1.38 mmol) at 0° C. under stirring was added triphosgene (74 mg, 0.25 mmol) portion-wise. The mixture was stirred for 4 h to afford crude 4-((R)-3-cyclopentyl-2-oxo-5-thiophen-3-yl-imidazolidin-1-yl)-piperidine-1-carboxylic acid tert-butyl ester as a yellow oil (140 mg, 53%).

Using general procedure C, the above substrate (140 mg, 0.334 mmol) and TFA (2 mL) in CH₂Cl₂ (4 mL) gave crude (R)-1-cyclopentyl-3-piperidin-4-yl-4-thiophen-3-yl-imidazolidin-2-one as white foam (70 mg, 66%).

Following general procedure A: to the above amine (70 mg, 0.22 mmol) was added 4-(5-formyl-pyrimidin-2-yloxy)-benzoic acid methyl ester (76 mg, 0.26 mmol), NaBH(OAc)₃ (65 mg, 0.31 mmol), HOAc (0.6 uL, 0.01 mmol) and CH₂Cl₂ (6 mL) and the mixture was stirred at room temperature overnight. Standard work-up and purification afforded 4-{5-[4-((R)-3-cyclopentyl-2-oxo-5-thiophen-3-yl-imidazolidin-1-yl)-piperidin-1-ylmethyl]-pyrimidin-2-yloxy}-benzoic acid methyl ester as a white foam (60 mg, 49%).

Following general procedure H, the above ester (60 mg, 0.11 mmol) was dissolved in THF/1N NaOH (5 mL:2 mL) and stirred at 50° C. for 8 hours to afford COMPOUND 328 as a white foam (25 mg, 43%). ¹H NMR (CD₃OD) δ 0.93 (br s, 1H), 1.33-1.70 (m, 9H), 1.85 (m, 2H), 2.16-2.19 (m, 1H), 2.38 (q, 2H, J=12.6 Hz), 3.02 (d, 1H, J=10.8 Hz), 3.12-3.21 (m, 2H), 3.53-3.61 (m, 1H), 3.71-3.75 (m, 3H), 4.29 (t, 1H, J=7.2 Hz), 7.14 (d, 1H, J=5.1 Hz), 7.30 (d, 2H, J=8.4 Hz), 7.44 (s, 1H), 7.48 (m, 1H), 8.13 (d, 2H, J=8.4 Hz), 8.59 (s, 2H).

### EXAMPLE 329

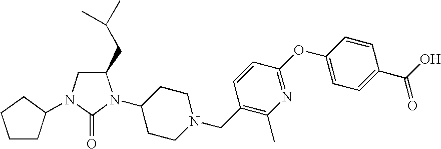

### COMPOUND 329: 4-{5-[4-((R)-3-Cyclopentyl-5-isobutyl-2-oxo-imidazolidin-1-yl)-piperidin-1-ylmethyl]-6-methyl-pyridin-2-yloxy}-benzoic acid

Following general procedure A, (R)-1-cyclopentyl-4-isobutyl-3-piperidin-4-yl-imidazolidin-2-one (89.3 mg, 0.304 mmol) and 4-(5-formyl-6-methyl-pyridin-2-yloxy)-benzoic acid tert-butyl ester (95 mg, 0.30 mmol) afforded 4-{5-[4-((R)-3-cyclopentyl-5-isobutyl-2-oxo-imidazolidin-1-yl)-piperidin-1-ylmethyl]-6-methyl-pyridin-2-yloxy}-benzoic acid tert-butyl ester. To the ester in THF (2 mL) was added 6N HCl (2 mL) and the mixture stirred at room temperature for 2 hours. Standard work-up and purification afforded COMPOUND 329 as a white solid (89 mg, 55% over 2 steps). ¹H NMR (CD₃OD) δ 1.00-1.03 (m, 6H), 1.42-1.80 (m, 11H), 1.94-2.05 (m, 2H), 2.24-2.38 (m, 1H), 2.51-2.60 (m, 4H), 3.03 (dd, 1H, J=8.7, 7.2 Hz), 3.12-3.24 (m, 2H), 3.53-3.76 (m, 5H), 4.15-4.25 (m, 1H), 4.37 (s, 2H), 6.97 (d, 1H, J=8.4 Hz), 7.24 (d, 2H, J=8.7 Hz), 7.98 (dd, 1H, J=8.4, 3.9 Hz), 8.11 (d, 2H, J=8.4 Hz); ES-MS m/z 535 (M+1). Anal. Calcd. for C₃₁H₄₂N₄O₄.0.76CH₂Cl₂.0.4CH₄O: C, 63.14; H, 7.43; N, 9.16. Found: C, 63.08; H, 7.56; N, 9.39.

### EXAMPLE 330

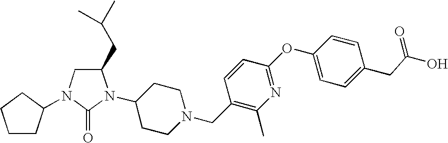

### COMPOUND 330: (4-{5-[4-((R)-3-Cyclolpentyl-5-isobutyl-2-oxo-imidazolidin-1-yl)-piperidin-1-ylmethyl]-6-methyl-pyridin-2-yloxy}-phenyl)-acetic acid

Following general procedure A, (R)-1-cyclopentyl-4-isobutyl-3-piperidin-4-yl-imidazolidin-2-one (63 mg, 0.21 mmol) and [4-(5-formyl-6-methyl-pyridin-2-yloxy)-phenyl]-acetic acid methyl ester (75%, 70 mg, 0.18 mmol) afforded (4-{5-[4-((R)-3-cyclopentyl-5-isobutyl-2-oxo-imidazolidin-1-yl)-piperidin-1-ylmethyl]-6-methyl-pyridin-2-yloxy}-phenyl)-acetic acid methyl ester. Following general procedure H, the methyl ester afforded COMPOUND 330 as a white solid (83 mg, 86% over 2 steps). ¹H NMR (CD₃OD) δ 0.96-0.99 (m, 6H), 1.37-1.98 (m, 13H), 2.15-2.27 (m, 1H), 2.38-2.46 (m, 1H), 2.51 (s, 3H), 2.97-3.07 (m, 3H), 3.46-3.71 (m, 7H), 4.13-4.18 (m, 1H), 4.22 (s, 2H), 6.77 (d, 1H, J=8.4 Hz), 7.07 (d, 2H, J=8.7 Hz), 7.35 (d, 2H, J=8.4 Hz), 7.83 (d, 1H, J=8.4 Hz); ES-MS m/z 549 (M+1). Anal. Calcd. for C₃₂H₄₄N₄O₄.0.58CH₂Cl₂.0.44CH₄O: C, 64.81; H, 7.73; N, 9.15. Found: C, 64.81; H, 7.72; N, 9.14.

### EXAMPLE 331

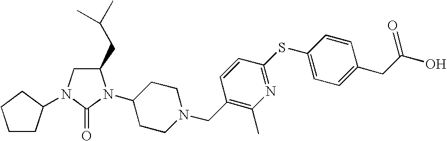

### COMPOUND 331: (4-{5-[4-((R)-3-Cyclopentyl-5-isobutyl-2-oxo-imidazolidin-1-yl)-piperidin-1-ylmethyl]-6-methyl-pyridin-2-ylsulfanyl}-phenyl)-acetic acid

Following general procedure A, (R)-1-cyclopentyl-4-isobutyl-3-piperidin-4-yl-imidazolidin-2-one (66 mg, 0.22 mmol) and [4-(5-formyl-6-methyl-pyridin-2-ylsulfanyl)-phenyl]-acetic acid (68 mg, 0.22 mmol) afforded (4-{5-[4-((R)-3-cyclopentyl-5-isobutyl-2-oxo-imidazolidin-1-yl)-piperidin-1-ylmethyl]-6-methyl-pyridin-2-ylsulfanyl}-phenyl)-acetic acid methyl ester. Following general procedure H, the methyl ester afforded COMPOUND 331 as a white solid (75 mg, 60% over 2 steps). ¹H NMR (CD₃OD) δ 0.95-0.98 (m, 6H), 1.36-1.43 (m, 1H), 1.52-1.94 (m, 11H), 2.10-2.23 (m, 1H), 2.32-2.45 (m, 1H), 2.58 (s, 3H), 2.86-3.01 (m, 3H), 3.37-3.40 (m, 2H), 3.47-3.56 (m, 2H), 3.63-3.68 (m, 3H), 4.11-4.18 (m, 3H), 4.65-4.74 (m, 1H), 6.75 (d, 1H, J=8.1 Hz), 7.42 (d, 2H, J=8.1 Hz), 7.53-7.59 (m, 3H); ES-MS m/z 565 (M+1). Anal. Calcd. for C₃₂H₄₄SN₄O_{3.}0.44CH₂Cl₂.0.73CH₄O: C, 63.67; H, 7.70; N, 8.95. Found: C, 63.70; H, 7.63; N, 8.83.

### EXAMPLE 332

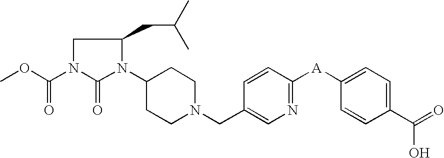

### COMPOUND 332: (R)-3-{1-[6-(4-Carboxy-phenylsulfanyl)-pyridin-3-ylmethyl]-piperidin-4-yl}-4-isobutyl-2-oxo-imidazolidine-1-carboxylic acid methyl ester

To a solution of ((R)-1-hydroxymethyl-3-methyl-butyl)-carbamic acid tert-butyl ester (4.288 g, 19.71 mmol), phthalimide (3.19 g, 21.7 mmol), Ph₃P (6.20 g, 23.7 mmol) in dry THF (135 mL) at 0° C. was added DEAD (3.41 mL, 21.7 mmol) dropwise. The mixture was then stirred at rt for 3 h. The solvent was removed by evaporation under reduced pressure.

Following general procedure C, the residue gave 2-((R)-2-amino-4-methyl-pentyl)-isoindole-1,3-dione (0.873 g, 18% over 2 steps).

Following general procedure A, the above product (698 mg, 2.84 mmol) was reacted with 1-Boc-4-piperidone (621 mg, 3.12 mmol) in the presence of NaBH(OAc)₃ (951 mg, 4.26 mmol) in CH₂Cl₂ (10 mL) for 5 h to give 4-[(R)-1-(1,3-dioxo-1,3-dihydro-isoindol-2-ylmethyl)-3-methyl-butylamino]-piperidine-1-carboxylic acid tert-butyl ester (331 mg, 27%).

To a solution of above product (331 mg, 0.772 mmol) in ethanol (5 mL) was added hydrazine hydrate (2 mL). The mixture was stirred at rt for 19 h. Standard work-up and purification by column chromatography on silica gel (CH₂Cl₂/MeOH/NH₄OH, 93:5:2) gave 4-((R)-1-aminomethyl-3-methyl-butylamino)-piperidine-1-carboxylic acid tert-butyl ester (186 mg, 81%).

To a solution of above product (186 mg, 0.622 mmol) in DMF (2 mL) was added carbonyl diimidazole (121 mg, 0.746 mmol). The mixture was stirred at rt for 2 h. Aqueous work-up and purification by column chromatography on silica gel (CH₂Cl₂/EtOAc, 1:1) gave 4-((R)-5-isobutyl-2-oxo-imidazolidin-1-yl)-piperidine-1-carboxylic acid tert-butyl ester as a white solid (198 mg, 98%).

To a solution of above product (134 mg, 0.412 mmol) in dry THF (4 mL) under N₂ was added NaH (60% dispersion in mineral oil, 49.6 mg, 1.24 mmol). After stirring at rt for 10 min, the mixture was cooled to 0° C. and methyl chloroformate (96 μL, 1.24 mmol) was added dropwise. The mixture was stirred at rt for 3 h. Aqueous work-up and purification by column chromatography on silica gel (CH₂Cl₂/EtOAc, 4:1 to 1:2) provided 4-((R)-5-isobutyl-3-methoxycarbonyl-2-oxo-imidazolidin-1-yl)-piperidine-1-carboxylic acid tert-butyl ester (122 mg, 77%).

Using general procedure C, the above product (122 mg, 0.319 mmol) in CH₂Cl₂ (3 mL) was treated with TFA (1 mL) to give (R)-4-isobutyl-2-oxo-3-piperidin-4-yl-imidazolidine-1-carboxylic acid methyl ester (90.1 mg, 100%). ¹H NMR (CDCl₃) δ 0.93 (d, 3H, J=6.6 Hz), 0.95 (d, 3H, J=6.6 Hz), 1.35-1.48 (m, 1H), 1.56-1.98 (m, 7H), 2.62-2.71 (m, 2H), 3.15 (t, 2H, J=12.9 Hz), 3.50 (dd, 1H, J=10.2, 4.2 Hz), 3.56-3.68 (m, 2H), 3.81 (dd, 1H, J=9.9, 8.7 Hz), 3.84 (s, 3H).

Following general procedure A, (R)-4-isobutyl-2-oxo-3-piperidin-4-yl-imidazolidine-1-carboxylic acid methyl ester (39.8 mg, 0.140 mmol) was coupled with 4-(5-formyl-pyridin-2-ylsulfanyl)-benzoic acid (57.2 mg, 0.211 mmol) in the presence of sodium triacetoxyborohydride (49.9 mg, 0.224 mmol) in CH₂Cl₂ (1.5 mL) to afford COMPOUND 332 as a white foam (22.8 mg, 31%). ¹H NMR (CDCl₃) δ 0.79 (d, 3H, J=6.0 Hz), 0.81 (d, 3H, J=6.0 Hz), 1.37 (t, 1H, J=10.8 Hz), 1.49-1.64 (m, 3H), 1.80 (d, 1H, J=10.8 Hz), 1.99 (d, 1H, J=11.4 Hz), 2.12-2.16 (m, 1H), 2.31-2.39 (m, 1H), 2.50-2.53 (m, 2H), 3.34 (d, 1H, J=10.8 Hz), 3.48 (dd, 2H, J=10.4, 4.4 Hz), 3.64 (m, 1H), 3.74-3.90 (m, 4H), 3.84 (s, 3H), 6.67 (d, 1H, J=8.7 Hz), 7.57 (d, 1H, J=8.1 Hz), 7.62 (d, 2H, J=8.4 Hz), 8.05 (d, 2H, J=8.4 Hz), 8.33 (d, 1H, J=1.5 Hz); ES-MS m/z 527 (M+H); Anal. Calcd. for C₂₇H₃₄N₄O₅S.0.6CH₂Cl₂: C, 57.39; H, 6.14; N, 9.70. Found: C, 57.12; H, 6.12; N, 9.39.

### EXAMPLE 333

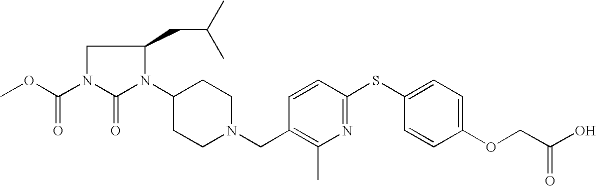

### COMPOUND 333: (R)-3-{1-[6-(4-Carboxymethoxy-phenylsulfanyl)-2-methyl-pyridin-3-ylmethyl]-piperidin-4-yl}-4-isobutyl-2-oxo-imidazolidine-1-carboxylic acid methyl ester

Following general procedure A, (R)-4-isobutyl-2-oxo-3-piperidin-4-yl-imidazolidine-1-carboxylic acid methyl ester (see EXAMPLE 332) (47.8 mg, 0.169 mmol) was coupled with [4-(5-formyl-6-methyl-pyridin-2-ylsulfanyl)-phenoxy]-acetic acid tert-butyl ester (72.7 mg, 0.202 mmol) in the presence of sodium triacetoxyborohydride (60.3 mg, 0.270 mmol) in CH₂Cl₂ (1.5 mL) to afford (R)-3-{1-[6-(4-tert-butoxycarbonylmethoxy-phenylsulfanyl)-2-methyl-pyridin-3-ylmethyl]-piperidin-4-yl}-4-isobutyl-2-oxo-imidazolidine-1-carboxylic acid methyl ester (89.4 mg, 84%).

The above ester (89.4 mg, 0.143 mmol) was treated with TFA (1 mL) in CH₂Cl₂ (2 mL) at rt for 2.5 h to yield COMPOUND 333 as a yellow foam (61.9 mg, 76%). ¹H NMR (CDCl₃) δ 0.92 (d, 3H, J=6.3 Hz), 0.94 (d, 3H, J=6.6 Hz), 1.39 (t, 1H, J=10.8 Hz), 1.59-1.69 (m, 2H), 1.82 (d, 1H, J=11.1 Hz), 1.99 (d, 1H, J=12.0 Hz), 2.14-2.27 (m, 1H), 2.36-2.47 (m, 1H), 2.56 (s, 3H), 2.76 (m, 2H), 3.42 (d, 1H, J=11.1 Hz), 3.59 (dd, 1H, J=10.2, 4.5 Hz), 3.61-3.68 (m, 2H), 3.80-3.88 (m, 1H), 3.84 (s, 3H), 3.95 (m, 1H), 3.98 (d, 1H, J=12.6 Hz), 4.10 (d, 1H, J=13.2 Hz), 4.48 (s, 2H), 6.37 (d, 1H, J=8.4 Hz), 7.01 (d, 2H, J=8.4 Hz), 7.49 (d, 2H, J=8.4 Hz), 7.57 (d, 1H, J=8.1 Hz); ¹³C NMR (CDCl₃) δ 21.52, 22.81, 24.40, 25.04, 26.27, 28.65, 44.20, 47.27, 49.77, 50.01, 51.55, 52.02, 56.40, 66.78, 116.57, 118.33, 120.92, 138.07, 140.38, 152.87, 153.71, 157.78, 159.96, 164.66, 173.51; ES-MS m/z 571 (M+H); Anal. Calcd. for C₂₉H₃₈N₄O₆S.1.0CH₂Cl₂: C, 54.96; H, 6.15; N, 8.55. Found: C, 54.74; H, 6.12; N, 8.40.

### EXAMPLE 334

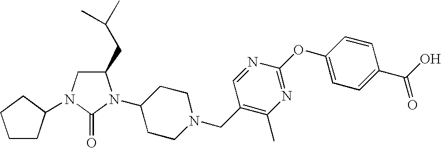

### COMPOUND 334: 4-{5-[4-((R)-3-Cyclopentyl-5-isobutyl-2-oxo-imidazolidin-1-yl)-piperidin-1-ylmethyl]-4-methyl-pyrimidin-2-yloxy}-benzoic acid

Following general procedure A, (R)-1-cyclopentyl-4-isobutyl-3-piperidin-4-yl-imidazolidin-2-one (65 mg, 0.22 mmol) and 4-(5-formyl-4-methyl-pyrimidin-2-yloxy)-benzoic acid methyl ester (see EXAMPLE 93) (60 mg, 0.22 mmol) afforded 4-{5-[4-((R)-3-cyclopentyl-5-isobutyl-2-oxo-imidazolidin-1-yl)-piperidin-1-ylmethyl]-4-methyl-pyrimidin-2-yloxy}-benzoic acid methyl ester. Following general procedure H, the methyl ester afforded COMPOUND 334 as a white solid (60 mg, 51% over 2 steps). ¹H NMR (CD₃OD) δ 0.95-0.98 (m, 6H), 1.37-1.44 (m, 1H), 1.50-1.89 (m, 14H), 2.00-2.13 (m, 1H), 2.20-2.32 (m, 1H), 2.56-2.64 (m, 5H), 2.94-3.01 (m, 1H), 3.25-3.31 (m, 2H), 3.47-3.52 (m, 2H), 3.63-3.71 (m, 1H), 3.92 (s, 2H), 4.13-4.17 (m, 1H), 4.63-4.65 (m, 1H), 7.27 (d, 2H, J=8.7 Hz), 8.09 (d, 2H, J=8.4 Hz), 8.47 (s, 1H); ES-MS m/z 536 (M+1). Anal. Calcd. for C₃₀H₄₁N₅O₄.0.45CH₂Cl₂.0.62CH₄O: C, 62.87; H, 7.53; N, 11.80. Found: C, 62.90; H, 7.46; N, 11.70.

### EXAMPLE 335

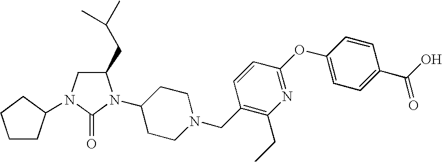

### COMPOUND 335: 4-{5-[4-((R)-3-Cyclopentyl-5-isobutyl-2-oxo-imidazolidin-1-yl)-piperidin-1-ylmethyl]-6-ethyl-pyridin-2-yloxy}-benzoic acid

Following general procedure A, (R)-1-cyclopentyl-4-isobutyl-3-piperidin-4-yl-imidazolidin-2-one (74 mg, 0.25 mmol) and 4-(6-ethyl-5-formyl-pyridin-2-yloxy)-benzoic acid tert-butyl ester (83 mg, 0.25 mmol) afforded 4-{5-[4-((R)-3-cyclopentyl-5-isobutyl-2-oxo-imidazolidin-1-yl)-piperidin-1-ylmethyl]-6-ethyl-pyridin-2-yloxy}-benzoic acid tert-butyl ester. Following general procedure C, the ester was treated with TFA (1 mL) in CH₂Cl₂ (2 mL). Standard work-up and purification afforded COMPOUND 335 as a white solid (74 mg, 53% over 2 steps). ¹H NMR (CD₃OD) δ 0.94-0.97 (m, 6H), 1.16 (t, 3H, J=7.2 Hz), 1.36-1.43 (m, 1H), 1.57-2.00 (m, 12H), 2.15-2.30 (m, 1H), 2.38-2.50 (m, 1H), 2.73-2.83 (m, 2H), 2.96-3.01 (m, 1H), 3.09-3.15 (m, 2H), 3.44-3.69 (m, 5H), 4.11-4.16 (m, 1H), 4.33 (s, 2H), 6.92 (d, 1H, J=8.4 Hz), 7.20-7.23 (m, 2H), 7.90 (d, 1H, J=8.4 Hz), 8.5-8.08 (m, 2H); ¹³C NMR (CD₃OD) δ 13.92, 22.13, 24.90, 25.42, 26.24, 28.34, 28.86, 29.48, 29.85, 45.19, 46.20, 50.93, 53.51, 53.81, 55.37, 57.41, 111.08, 120.27, 122.11, 129.08, 132.95, 145.63, 159.66, 162.34, 164.14, 164.79, 170.30; ES-MS m/z 549 (M+1). Anal. Calcd. for C₃₂H₄₄N₄O₄.1.2CH₂Cl₂: C, 56.33; H, 6.68; N, 7.71. Found: C, 56.20; H, 6.35; N, 7.46.

### EXAMPLE 336

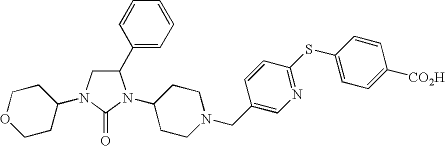

### COMPOUND 336: 4-(5-{4-[2-Oxo-5-phenyl-3-(tetrahydro-pyran-4-yl)-imidazolidin-1-yl]-piperidin-1-ylmethyl}-1-pyridin-2-ylsulfanyl)-benzoic acid

Using general procedure A, 1-phenyl-N²-(tetrahydro-pyran-4-yl)-ethane-1,2-diamine (2.89 g, 13.1 mmol), 1-boc-4-piperidone (2.61 g, 13.1 mmol), glacial AcOH (0.752 mL, 13.1 mmol) and NaBH(OAc)₃ (3.89 mg, 18.3 mmol) in CH₂Cl₂ (10 mL) gave crude 4-[1-phenyl-2-(tetrahydro-pyran-4-ylamino)-ethylamino]-piperidine-1-carboxylic acid tert-butyl ester as a yellow oil (5.29 g, quant).

Following general procedure K: to a solution of the above diamine (5.29 mg, 13.1 mmol) in CH₂Cl₂ (50 mL) and pyridine (2.32 mL, 28.8 mmol) at 0° C. under stirring was added triphosgene (1.55 g, 5.24 mmol) portion-wise. The mixture was stirred for 4 h to afford crude 4-[2-oxo-5-phenyl-3-(tetrahydro-pyran-4-yl]-imidazolidin-1-yl]-piperidine-1-carboxylic acid tert-butyl ester as a yellow oil (5.75 g, quant).

Using general procedure C, the above substrate (5.75 g, 13.3 mmol) and TFA (2 mL) in CH₂Cl₂ (4 mL) gave crude 4-phenyl-3-piperidin-4-yl-1-(tetrahydro-pyran-4-yl)-imidazolidin-2-one as yellow foam (3.89 g, 90%).

Following general procedure A: to the above amine (1.45 g, 4.53 mmol) was added 4-(5-formyl-pyridin-2-ylsulfanyl)-benzoic acid methyl ester (1.45 g, 5.43 mmol), NaBH(OAc)₃ (1.01 g, 6.34 mmol), HOAc (9 μL, 0.2 mmol) and CH₂Cl₂ (30 mL) and the mixture was stirred at room temperature overnight. Standard work-up and purification afforded 4-(5-{4-[2-oxo-5-phenyl-3-(tetrahydro-pyran-4-yl)-imidazolidin-1-yl]-piperidin-1-ylmethyl}-pyridin-2-ylsulfanyl)-benzoic acid methyl ester as a white foam (2.0 g, 75%).

Using general procedure H, the above ester (2.0 g, 3.4 mmol) and 1N NaOH (25 mL) in MeOH (25 mL) gave COMPOUND 336 as a white foam (1.75 g, 90%). ¹H NMR (CDCl₃) δ 1.50 (s, 2H), 1.65-1.71 (m, 4H), 1.79-1.83 (m, 1H), 2.30-2.50 (m, 3H), 3.07-3.12 (m, 2H), 3.42-3.51 (m, 3H), 3.62-3.78 (m, 3H), 3.96-4.04 (m, 4H), 4.55-4.61 (m, 1H), 6.67-6.69 (m, 1H), 7.18 (d, 3H, J=3 Hz), 7.25 (d, 2H, J=3 Hz), 7.61 (d, 3H, J=9 Hz), 7.99 (d, 2H, J=9 Hz), 8.26 (s, 1H).

### EXAMPLE 337

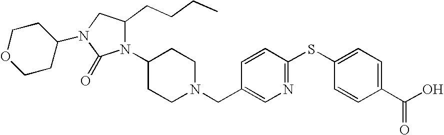

### COMPOUND 337: 4-(5-{4-[5-Butyl-2-oxo-3-(tetrahydro-pyran-4-yl)-imidazolidin-1-yl]-piperidin-1-ylmethyl}-pyridin-2-ylsulfanyl)-benzoic acid

D,L-Leucine (4.00 g, 30.5 mmol) was dissolved in THF (150 mL) and treated with BH₃.THF (150 mL), heating to 60° C. and stirring for 16 h. Methanol (10 mL) was added, and the solution stirred another 10 minutes. The solvent was then removed under reduced pressure and the residue dissolved in methanol (30 mL). Ethylene diamine (5 mL) in methanol (5 mL) was added and the solution was heated to reflux for 30 minutes. Standard work-up and purification by column chromatography (20:1, CH₂Cl₂/MeOH) gave 2-amino-hexan-1-ol as a colorless oil (1.68 g, 47%).

The amine from above (1.68 g, 14.3 mmol) was dissolved in CH₂Cl₂ (70 mL) and treated with diisopropylamine (5.0 mL) and di-tert-butyl dicarbonate (4.68 g, 21.4 mmol) for 16 h. Very little product was forming by tlc, so another portion of di-tert-butyl dicarbonate (2.3 g, 10.5 mmol) was added and the reaction was stirred for another 5 days at room temperature. Standard work-up and purification by column chromatography (1:2, EtOAc/hexanes) gave (1-hydroxymethyl-pentyl)-carbamic acid tert-butyl ester as a colorless oil (0.47 g, 15%).

The above alcohol (0.47 g, 2.2 mmol) was then converted to an amine using the general procedures for the Mitsunobu and Phthalimide deprotection reactions to give, after column chromatography with silica gel (20:1:0.1, CH₂Cl₂/MeOH/NH₄OH), (1-aminomethyl-pentyl)-carbamic acid tert-butyl ester as a brown oil (0.37 g, 78% over 2 steps).

Using general procedure A, the above amine (0.37 g, 1.7 mmol) and tetrahydro-pyran-4-one (0.17 mL, 1.8 mmol) were reacted to give {1-[(tetrahydro-pyran-4-ylamino)-methyl]-pentyl}-carbamic acid tert-butyl ester as a brown oil (0.39 g, 76%). Following general procedure C, the crude product provided N¹-(tetrahydro-pyran-4-yl)-hexane-1,2-diamine as a pale yellow solid (0.24 g, 91%).

Using general procedure A, the above diamine (0.24 g, 1.2 mmol) and N-Boc-4-piperidone (0.25 g, 1.3 mmol) were reacted to give, after standard work-up and chromatography with silica gel (50:1:0.1, CH₂Cl₂/MeOH/NH₄OH), 4-{1-[(tetrahydro-pyran-4-ylamino)-methyl]-pentylamino}-piperidine-1-carboxylic acid tert-butyl ester as a pale brown oil (0.32 g, 69%).

Following general procedure K, to a cooled (0° C.) solution of the above compound (0.32 g, 0.82 mmol) and pyridine (0.10 mL, 1.2 mmol) in dry dichloromethane (4 mL) was slowly added triphosgene (0.12 g, 0.41 mmol). The ice bath was removed and the mixture was gradually warmed to ambient temperature over 1 h to afford 4-[5-butyl-2-oxo-3-(tetrahydro-pyran-4-yl)-imidazolidin-1-yl]-piperidine-1-carboxylic acid tert-butyl ester as a yellow solid. Following general procedure C, the crude product provided 4-butyl-3-piperidin-4-yl-1-(tetrahydro-pyran-4-yl)-imidazolidin-2-one as a pale yellow solid (0.23 g, 90% over 2 steps). ¹H NMR (CDCl₃) δ 0.92 (t, 3H, J=6.9 Hz), 1.27 (m, 4H), 1.50 (m, 1H), 1.63 (m, 5H), 1.81 (m, 4H), 2.67 (dq, 2H, J=12.0, 3.0 Hz), 2.91 (m, 1H), 3.14 (t, 2H, J=12.0 Hz), 3.36 (t, 1H, J=10.5 Hz), 3.48 (t, 2H, J=11.4 Hz), 1.65 (m, 2H), 4.00 (m, 3H).

Following general procedure A: the above secondary amine (46 mg, 0.15 mmol) and 4-(5-formyl-pyridin-2-ylsulfanyl)-benzoic acid methyl ester (53 mg, 0.20 mmol) were combined in CH₂Cl₂ (1.5 mL) and treated with sodium triacetoxyborohydride (57 mg, 0.27 mmol) at room temperature for 16 h. After standard work-up, the crude material was purified by flash column chromatography on silica gel (EtOAc) to afford 4-(5-{4-[5-butyl-2-oxo-3-(tetrahydro-pyran-4-yl)-imidazolidin-1-yl]-piperidine-1-ylmethyl}-pyridin-2-ylsulfanyl)-benzoic acid methyl ester as a white solid (70 mg, 82%).

Following general procedure H, the above ester (70 mg, 0.12 mmol) gave COMPOUND 337 as a white solid (65 mg, 95%). ¹H NMR (CD₃OD) δ 0.96 (t, 3H, J=6.9 Hz), 1.40 (m, 4H), 1.50 (m, 1H), 1.58 (d, 2H, J=12.3 Hz), 1.78 (m, 4H), 1.96 (d, 1H, J=12.0 Hz), 2.17 (dq, 1H, J=12.0, 3.6 Hz), 2.40 (dq, 1H, J=12.0, 3.6 Hz), 2.81 (q, 2H, J=11.5 Hz), 3.07 (m, 1H), 3.39 (m, 2H), 3.51 (m, 3H), 3.58 (tt, 2H, J=10.5, 4.5 Hz), 3.64 (q, 1H, J=11.4 Hz), 3.83 (tt, 1H, J=10.5, 4.5 Hz), 4.02 (dd, 2H, J=11.4, 3.6 Hz), 4.10 (s, 2H), 7.18 (d, 1H, J=8.4 Hz), 7.67 (d, 2H, J=8.4 Hz), 7.78 (dd, 1H, J=8.4, 2.1 Hz), 8.10 (d, 2H, J=8.1 Hz), 8.49 (d, 1H, J=1.8 Hz); ES-MS m/z 553 (M+H).

### EXAMPLE 338

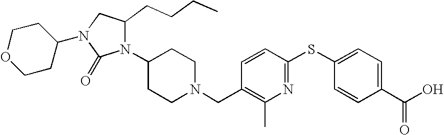

### COMPOUND 338: 4-(5-{4-[5-Butyl-2-oxo-3-(tetrahydro-pyran-4-yl)-imidazolidin-1-yl]-piperidin-1-ylmethyl}-6-methyl-pyridin-2-ylsulfanyl)-benzoic acid

Following general procedure A: 4-butyl-3-piperidin-4-yl-1-(tetrahydro-pyran-4-yl)-imidazolidin-2-one (see EXAMPLE 337) (46 mg, 0.15 mmol) and 4-(5-formyl-6-methyl-pyridin-2-ylsulfanyl)-benzoic acid methyl ester (56 mg, 0.20 mmol) were combined in CH₂Cl₂ (1.5 mL) and treated with sodium triacetoxyborohydride (57 mg, 0.27 mmol) at room temperature for 16 h. After standard work-up, the crude material was purified by flash column chromatography on silica gel (EtOAc) to afford 4-(5-{4-[5-butyl-2-oxo-3-(tetrahydro-pyran-4-yl)-imidazolidin-1-yl]-piperidine-1-ylmethyl}-6-methyl-pyridin-2-ylsulfanyl)-benzoic acid methyl ester as a white solid (64 mg, 73%).

Following general procedure H, the above ester (63 mg, 0.11 mmol) gave COMPOUND 338 as a white solid (55 mg, 90%). ¹H NMR (CD₃OD) δ 0.98 (t, 3H, J=6.9 Hz), 1.30-1.55 (m, 5H), 1.58 (d, 2H, J=10.5 Hz), 1.80 (q, 3H, J=12.0 Hz), 1.98 (m, 2H), 2.25 (dq, 1H, J=12.0, 3.6 Hz), 2.52 (dq, 1H, J=12.0, 3.6 Hz), 2.63 (s, 3H), 3.08 (q, 2H, J=11.5 Hz), 3.08 (m, 1H), 3.45-3.75 (m, 7H), 3.86 (tt, 1H, J=10.5, 4.5 Hz), 4.00 (dd, 2H, J=11.7, 4.2 Hz), 4.29 (s, 2H), 7.01 (d, 1H, J=8.4 Hz), 7.68 (d, 2H, J=8.4 Hz), 7.72 (d, 1H, J=8.1 Hz), 8.11 (d, 2H, J=8.4 Hz); ES-MS m/z 567 (M+H).

### EXAMPLE 339

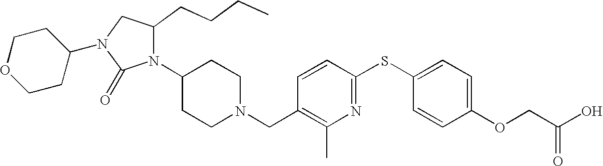

### COMPOUND 339: [4-(5-{4-[5-Butyl-2-oxo-3-(tetrahydro-pyran-4-yl)-imidazolidin-1-yl]-piperidin-1-ylmethyl}-6-methyl-pyridin-2-ylsulfanyl)-phenoxy]-acetic acid

Following general procedure A: 4-butyl-3-piperidin-4-yl-1-(tetrahydro-pyran-4-yl)-imidazolidin-2-one (see EXAMPLE 337) (31 mg, 0.10 mmol) and [4-(5-formyl-6-methyl-pyridin-2-ylsulfanyl)-phenoxy]-acetic acid tert-butyl ester (47 mg, 0.13 mmol) were combined in CH₂Cl₂ (1 mL) and treated with sodium triacetoxyborohydride (38 mg, 0.18 mmol) at room temperature for 16 h. After standard work-up, the crude material was purified by flash column chromatography on silica gel (EtOAc) to afford [4-(5-{4-[5-butyl-2-oxo-3-(tetrahydro-pyran-4-yl)-imidazolidin-1-yl]-piperidin-1-ylmethyl}-6-methyl-pyridin-2-ylsulfanyl)-phenoxy]-acetic acid tert-butyl ester as a white solid (54 mg, 83%).

Following general procedure C, the ester from above (54 mg, 93 μmol) was dissolved in CH₂Cl₂ (1 mL) and treated with TFA (0.4 mL), stirring for 16 h. Standard work-up and purification afforded COMPOUND 339 as a white solid (48 mg, 97%). ¹H NMR (CD₃OD) δ 0.98 (t, 3H, J=6.9 Hz), 1.30-1.50 (m, 5H), 1.56 (m, 2H), 1.76 (q, 3H, J=12.0 Hz), 1.92 (m, 2H), 2.25 (dq, 1H, J=12.0, 3.6 Hz), 2.52 (dq, 1H, J=12.0, 3.6 Hz), 2.62 (s, 3H), 3.08 (m, 1H), 3.20 (q, 2H, J=11.5 Hz), 3.40-3.62 (m, 6H), 3.67 (q, 1H, J=10.5 Hz), 3.83 (tt, 1H, J=12.6, 4.5 Hz), 4.00 (dd, 2H, J=11.4, 3.9 Hz), 4.34 (s, 2H), 4.77 (s, 2H), 6.71 (d, 1H, J=8.1 Hz), 7.11 (d, 2H, J=9.0 Hz), 7.57 (d, 2H, J=8.7 Hz), 7.62 (d, 1H, J=8.4 Hz); ES-MS m/z 597 (M+H).

### EXAMPLE 340

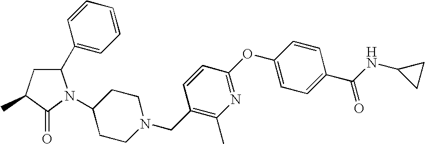

### COMPOUND 340: N-Cyclopropyl-4-{6-methyl-5-[4-((S)-3-methyl-2-oxo-5-phenyl-pyrrolidin-1-yl)-piperidin-1-ylmethyl]-pyridin-2-yloxy}-benzamide

To a solution of 4-amino-1-Boc-piperidine (1.01 g, 5.05 mmol) and DIPEA (0.60 mL, 7.5 mmol) in THF (25 mL) was slowly added methacryloyl chloride (0.60 mL, 6.1 mmol) and the resulting solution was stirred at room temperature for 60 minutes. Standard work-up and purification gave 4-(2-methyl-acryloylamino)-piperidine-1-carboxylic acid tert-butyl ester as a white solid (967 mg, 71%).

To a −78° C. solution of the methacrylamide (273 mg, 1.02 mmol) and TMEDA (0.30 mL, 2.0 mmol) in THF (3.0 mL) under nitrogen was added n-BuLi (2.5M in hexanes, 0.95 mL, 2.4 mmol) (Fitt, J. J., et al., J. Org. Chem. (1980) 45:4257-4259). The reaction was warmed to −20° C., stirred for 45 minutes and then a solution of benzaldehyde (0.11 mL, 1.1 mmol) in THF (1.0 mL) was added. The reaction was warmed to room temperature and stirred for another 1.5 hours. Standard work-up and purification gave 4-(4-hydroxy-2-methylene-4-phenyl-butyrylamino)-piperidine-1-carboxylic acid tert-butyl ester as a white foam (210 mg, 55%).

To a −78° C. solution of the amide (206 mg, 0.55 mmol) in THF (3.5 mL) under nitrogen was added n-BuLi (2.5M in hexanes, 0.50 mL, 1.2 mmol) and the resulting yellow solution was stirred at −78° C. for 30 minutes (Tanaka, K.; Yoda, H.; Kaji, A. Synthesis, 1985, 84-86). A solution of p-TsCl (123 mg, 0.64 mmol) in THF (1.5 mL) was added and the reaction was warmed to room temperature and stirred for an additional 18.5 hours. Standard work-up and purification gave 4-(3-methylene-2-oxo-5-phenyl-pyrrolidin-1-yl)-piperidine-1-carboxylic acid tert-butyl ester as a beige foam (158 mg, 81%).

A mixture of the alkene (158 mg, 0.44 mmol) and 10% Pd/C (50% H₂O, 48 mg, 0.023 mmol) in MeOH (3.0 mL) was stirred at room temperature under H₂ (1 atm) for 2 hours. The mixture was filtered through a cotton plug, washing with MeOH. The filtrate was concentrated under reduced pressure and purification gave 4-(3-methyl-2-oxo-5-phenyl-pyrrolidin-1-yl)-piperidine-1-carboxylic acid tert-butyl ester as a white foam (125 mg). Following general procedure C, the tert-butyl carbamate (125 mg, 0.35 mmol) gave 3-methyl-5-phenyl-1-piperidin-4-yl-pyrrolidin-2-one as a yellow oil (53.0 mg, 59%). ¹H NMR (CDCl₃) δ 1.25-1.39 (m, 1H), 1.28 (d, 3H, J=7.0 Hz), 1.45-1.65 (m, 4H), 1.94 (qd, 1H, J=12.2, 4.2 Hz), 2.41-2.54 (m, 3H), 2.63 (ddd, 1H, J=12.7, 9.4, 7.4 Hz), 2.88-2.95 (m, 1H), 2.99-3.06 (m, 1H), 3.62 (tt, 1H, J=12.1, 3.9 Hz), 4.56 (t, 1H, J=7.7 Hz), 7.25-7.38 (m, 5H).

Following general procedure A, the above amine (71 mg, 0.28 mmol) and N-cyclopropyl-4-(5-formyl-6-methyl-pyridin-2-yloxy)-benzamide (81 mg, 0.28 mmol) afforded COMPOUND 340 as a white solid (153 mg, 95%). ¹H NMR (CDCl₃) δ 0.79-0.85 (m, 2H), 1.25-1.28 (m, 3H), 1.42-1.58 (m, 5H), 1.84-2.09 (m, 4H), 2.33 (s, 3H), 1.43-1.51 (m, 1H), 2.58-2.69 (m, 2H), 2.72-2.79 (m, 1H), 2.85-2.91 (m, 1H), 3.28 (s, 2H), 3.51-3.59 (m, 1H), 4.54 (t, 1H, J=7.8 Hz), 6.41 (br s, 1H), 6.58 (d, 1H, J=8.1 Hz), 7.09 (d, 2H, J=8.4 Hz), 7.25-7.37 (m, 5H), 7.47 (d, 1H, J=8.4 Hz), 7.74 (d, 2H, J=8.4 Hz); ¹³C NMR (CDCl₃) δ 6.77, 16.98, 21.79, 23.14, 28.43, 29.00, 29.68, 36.97, 38.53, 52.69, 53.16, 58.76, 60.70, 108.43, 119.91, 127.06, 127.71, 128.05, 128.55, 128.73, 129.89, 140.95, 143.14, 156.57, 157.71, 160.75, 168.31, 178.59; ES-MS m/z 561 (M+Na). Anal. Calcd. for C₃₃H₃₈N₄O₃.0.39CH₄O.0.24CH₂Cl₂: C, 70.67; H, 7.06; N, 9.80. Found: C, 70.65; H, 7.10; N, 9.88.

### EXAMPLE 341

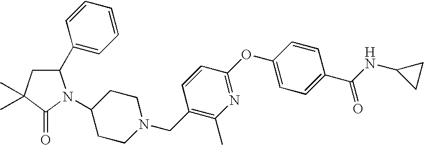

### COMPOUND 341: N-Cyclopropyl-4-{5-[4-(3,3-dimethyl-2-oxo-5-phenyl-pyrrolidin-1-yl)-piperidin-1-ylmethyl]-6-methyl-pyridin-2-yloxy}-benzamide

To a −78° C. solution of the methyl isobutyrate (0.51 mL, 4.5 mmol) in THF (15 mL) was added n-BuLi (2.3M in hexanes, 2.2 mL, 5.1 mmol) and the mixture was stirred at −78° C. for 1 hour. TMEDA (0.78 mL, 4.8 mmol) was added followed by (2-iodoethyl)benzene (1.0 mL, 6.8 mmol). The reaction was stirred at −78° C. for 2 hours then warmed to room temperature. Standard work-up and purification gave 2,2-dimethyl-4-phenyl-butyric acid methyl ester as a white foam (730 mg). N₂ was bubbled through a solution of the ester, NBS (628 mg, 3.53 mmol) and peroxide (85 mg, 0.35 mmol) in CCl₄ for 5 minutes. The mixture was heated to 85° C. for 2 hours, cooled to room temperature and filtered. The filtrate was washed with hexanes and dried in vacuo to afford the crude bromide (850 mg). A solution of the bromide, 4-amino-piperidine-1-carboxylic acid tert-butyl ester (596 mg, 2.98 mmol), N,N-diisopropylethylamine (0.7 mL) in CH₃CN (29 mL) was heated to 85° C. overnight. Standard work-up afforded the desired carbamate. Following general procedure C, the carbamate afforded 3,3-dimethyl-5-phenyl-1-piperidin-4-yl-pyrrolidin-2-one (363 mg, 30% over 4 steps).

Following general procedure A, the above amine (40 mg, 0.15 mmol) and N-cyclopropyl-4-(5-formyl-6-methyl-pyridin-2-yloxy)-benzamide (44 mg, 0.15 mmol) afforded COMPOUND 341 as a white solid (20 mg, 25%). ¹H NMR (CDCl₃) δ 0.84-0.86 (m, 2H), 1.28 (d, 6H, J=9.9 Hz), 1.60-1.77 (m, 4H), 1.84-1.92 (m, 1H), 2.07-2.15 (m, 2H), 2.31 (dd, 1H, J=12.3, 5.7 Hz), 2.44 (s, 3H), 2.79-2.89 (m, 3H), 3.40 (s, 2H), 3.54-3.63 (m, 1H), 5.25-5.31 (m, 1H), 6.33 (br s, 1H), 6.63 (d, 1H, J=8.1 Hz), 7.10-7.13 (m, 2H), 7.30-7.40 (m, 5H), 7.60 (d, 1H, J=8.1 Hz), 7.74 (d, 2H, J=8.7 Hz); ¹³C NMR (CDCl₃) δ 5.75, 20.81, 22.10, 25.22, 25.36, 31.89, 32.08, 39.68, 46.88, 51.52, 52.26, 58.26, 77.73, 107.47, 118.85, 124.46, 125.34, 126.99, 127.16, 127.51, 127.58, 128.78, 139.96, 155.60, 156.81, 159.65, 165.98, 167.30; ES-MS m/z 553 (M+1).

### EXAMPLE 342

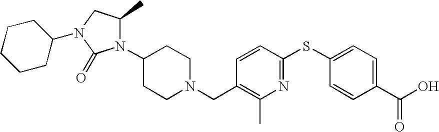

### COMPOUND 342: 4-{5-[4-((R)-3-Cyclohexyl-5-methyl-2-oxo-imidazolidin-1-yl)-piperidin-1-ylmethyl]-6-methyl-pyridin-2-ylsulfanyl}-benzoic acid

Boc-D-alanine (2.00 g, 10.6 mmol) was coupled with cyclohexylamine (1.10 mL, 9.6 mmol) using general procedure E. After standard work-up and chromatographic purification on silica gel (2:1, hexanes/EtOAc to EtOAc), (1-cyclohexylcarbamoyl-ethyl)-carbamic acid tert-butyl ester was obtained as white solid (1.45 g, 56%).

The above compound (1.45 g, 5.4 mmol) was treated with TFA in CH₂Cl₂ under conditions of general procedure C to provide, after standard work-up, 2-amino-N-cyclohexyl-propionamide as a brown oil (0.91 g). The crude material was then dissolved in THF (25 mL) and treated with BH₃.THF (25 mL), heating to 60° C. and stirring for 16 h. Methanol (6 mL) was added, and the solution stirred another 10 minutes. The solvent was then removed under reduced pressure and the residue dissolved in methanol (30 mL). Ethylene diamine (10 mL) was added and the solution was heated to reflux for 30 minutes. Standard work-up and purification afforded N¹-cyclohexyl-propane-1,2-diamine as a colorless oil (0.62 g, 74% over 2 steps).

Using general procedure A, the amine from above (0.62 g, 4.0 mmol) and N-Boc-4-piperidone (0.83 g, 4.2 mmol) gave 4-(2-cyclohexylamino-1-methyl-ethylamino)-piperidine-1-carboxylic acid tert-butyl ester as a colorless oil (1.05 g, 78%).

Following general procedure K, to a cooled (0° C.) solution of the above compound (1.05 g, 3.1 mmol) and pyridine (0.37 mL, 4.6 mmol) in dry dichloromethane (15 mL) was slowly added triphosgene (0.46 g, 1.6 mmol). The ice bath was removed and the mixture was gradually warmed to ambient temperature over 1 h. Saturated aqueous NH₄Cl solution (20 mL) was added and the mixture was shaken in a separatory funnel. The layers were separated and the resulting aqueous layer was extracted with dichloromethane (2×20 mL). The combined organic layers were dried (Na₂SO₄) and concentrated in vacuo to afford 4-(3-cyclohexyl-5-methyl-2-oxo-imidazolidin-1-yl)-piperidine-1-carboxylic acid tert-butyl ester as a yellow solid. The crude product was then treated under conditions of general procedure C to provide, after standard work-up and column chromatography (20:1:0.1, CH₂Cl₂/MeOH/NH₄OH), (R)-1-cyclohexyl-4-methyl-3-piperidin-4-yl-imidazolidin-2-one as a pale yellow solid (0.59 g, 100% over 2 steps). ¹H NMR (CDCl₃) δ 1.05 (m, 1H), 1.26 (d, 3H, J=6.3 Hz), 1.34 (m, 4H), 1.70-1.85 (m, 9H), 2.66 (dq, 2H, J=12.0, 3.0 Hz), 2.78 (m, 1H), 3.10 (t, 2H, J=11.4 Hz), 3.36 (t, 1H, J=9.0 Hz), 3.69 (m, 3H).

Following General procedure A: the above amine (39 mg, 0.15 mmol) and 4-(5-formyl-6-methyl-pyridin-2-ylsulfanyl)-benzoic acid methyl ester (50 mg, 0.17 mmol) were combined in CH₂Cl₂ (2 mL) and treated with sodium triacetoxyborohydride (50 mg, 0.23 mmol) at room temperature for 16 h. After standard work-up, the crude material was purified by flash column chromatography on silica gel (1:2, EtOAc/hexanes) to afford (R)-4-{5-[4-(3-cyclohexyl-5-methyl-2-oxo-imidazolidin-1-yl)-piperidin-1-ylmethyl]-6-methyl-pyridin-2-ylsulfanyl}-benzoic acid methyl ester as a white solid (57 mg, 73%).

Following general procedure H, the above ester (55 mg, 0.10 mmol) afforded COMPOUND 342 as a white solid (28 mg, 54%). ¹H NMR (CDCl₃) δ 1.12 (q, 1H, J=10.2 Hz), 1.24 (d, 3H, J=6.0 Hz), 1.35 (m, 4H), 1.66 (m, 4H), 1.80 (m, 3H), 1.95 (dq, 1H, J=12.3, 3.6 Hz), 2.12 (dq, 1H, J=12.3, 3.6 Hz), 2.39 (q, 2H, J=10.8 Hz), 2.55 (s, 3H), 2.87 (m, 1H), 3.09 (t, 2H, J=11.4 Hz), 3.46 (t, 1H, J=8.7 Hz), 3.56 (m, 2H), 3.70 (s, 2H), 3.74 (m, 1H), 6.88 (d, 1H, J=8.1 Hz), 7.57 (m, 3H), 8.02 (d, 2H, J=8.4 Hz); ES-MS m/z 523 (M+H).

### EXAMPLE 343

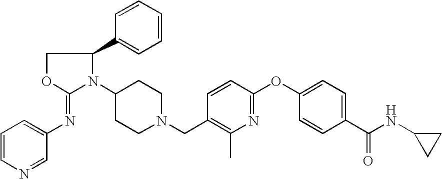

### COMPOUND 343: N-Cyclopropyl-4-[6-methyl-5-(4-{(R)-4-phenyl-2-[pyridin-3-ylimino]-oxazolidin-3-yl}-piperidin-1-ylmethyl)-pyridin-2-yloxy]-benzamide

To a solution of 4-[(R)-2-hydroxy-1-phenyl-ethylamino]-piperidine-1-carboxylic acid tert-butyl ester (682 mg, 2.13 mmol) in CH₂Cl₂ (6 mL) was added 3-pyridylisocyanate (268 mg, 2.24 mmol) and the mixture was stirred at rt for 1 h. Solvent was evaporated and the residue was purified by column chromatography on silica gel (2-10% MeOH/CH₂Cl₂) to provide 4-{1-[(R)-2-hydroxy-1-phenyl-ethyl]-3-pyridin-3-yl-ureido}-piperidine-1-carboxylic acid tert-butyl ester (526 mg, 56%).

To a solution of 4-{1-[(R)-2-hydroxy-1-phenyl-ethyl]-3-pyridin-3-yl-ureido}-piperidine-1-carboxylic acid tert-butyl ester (573 mg, 1.302 mmol) in CH₂Cl₂ (10 mL) and triethylamine (197, mg, 1.95 mmol) was added methanesulfonylchloride (157 mg, 1.367 mmol) and the mixture was stirred at rt for 15 min and then heated at reflux for 30 min. Solvent was evaporated and the residue was purified by column chromatography on silica gel (1:1, hexane/EtOAc and 3-5% MeOH/CH₂Cl₂) to provide 4-[(R)-4-phenyl-2-(pyridin-3-ylimino)-oxazolidin-3-yl]-piperidine-1-carboxylic acid tert-butyl ester (387 mg, 70%).

Following general procedure C: the above carbamate (387 mg, 0.917 mmol) in CH₂Cl₂ (5 mL) was treated with TFA (1.5 mL) at rt for 1 h to give [(R)-4-phenyl-3-piperidin-4-yl-oxazolidin-2-ylidene]-pyridin-3-yl-amine (295 mg, 100%). ¹H NMR (CDCl₃) δ 1.07-1.22 (m, 1H), 1.60 (d, 1H, J=12.0 Hz), 1.74-1.85 (m, 2H), 2.28 (br s, 1H), 2.52 (t, 1H, J=12.0 Hz), 2.65 (t, 1H, J=11.4 Hz), 2.89 (d, 1H, J=12.0 Hz), 3.10 (d, 1H, J=12.0 Hz), 3.94 (tt, 1H, J=11.7, 3.9 Hz), 4.07 (dd, 1H, J=8.4, 5.4 Hz), 4.54 (t, 1H, J=8.4 Hz), 4.79 (dd, 1H, J=8.4, 5.4 Hz), 7.12 (dd, 1H, J=8.1, 4.5 Hz), 7.28-7.42 (m, 6H), 8.15 (d, 1H, J=3.9 Hz), 8.38 (s, 1H).

Following general procedure A, N-cyclopropyl-4-(5-formyl-6-methyl-pyridin-2-yloxy)-benzamide (50.7 mg, 0.171 mmol) was reacted with [(R)-4-phenyl-3-piperidin-4-yl-oxazolidin-2-ylidene]-pyridin-3-yl-amine (46.0 mg, 0.143 mmol) in the presence of NaBH(OAc)₃ (51.1 mg, 0.229 mmol) in dichloromethane (1.5 mL) to provide COMPOUND 343 as a white foam (62.5 mg, 73%). ¹H NMR (CDCl₃) δ 0.57-0.63 (m, 2H), 0.81-0.87 (m, 2H), 1.16-1.30 (m, 1H), 1.57 (d, 1H, J=12.3 Hz), 1.87-2.16 (m, 4H), 2.37 (s, 3H), 2.68 (d, 1H, J=11.1 Hz), 2.85-2.92 (m, 2H), 3.34 (s, 2H), 3.92 (m, 1H), 4.11 (dd, 1H, J=8.4, 5.1 Hz), 4.57 (t, 1H, J=8.4 Hz), 4.81 (dd, 1H, J=8.4, 5.1 Hz), 6.48 (br s, 1H), 6.59 (d, 1H, J=8.1 Hz), 7.09 (d, 2H, J=8.7 Hz), 7.14-7.18 (m, 1H), 7.31-7.41 (m, 5H), 7.45 (d, 1H, J=8.4 Hz), 7.49 (d, 1H, J=8.4 Hz), 7.74 (d, 2H, J=8.7 Hz), 8.18 (br s, 1H), 8.40 (br s, 1H); ¹³C NMR (CDCl₃) δ 5.74, 20.94, 22.27, 27.99, 28.77, 29.44, 52.01, 52.14, 52.41, 57.89, 57.94, 72.34, 107.51, 119.01, 122.37, 125.75, 126.48, 127.79, 128.22, 129.13, 129.54, 140.09, 140.19, 141.62, 143.41, 144.73, 152.99, 155.72, 156.64, 159.99, 167.50; ES-MS m/z 603 (M+H). Anal. Calcd. for C₃₆H₃₈N₆O₃.0.3CH₂Cl₂: C, 69.40; H, 6.19; N, 13.38. Found: C, 69.56; H, 6.38; N, 13.28.

### EXAMPLE 344

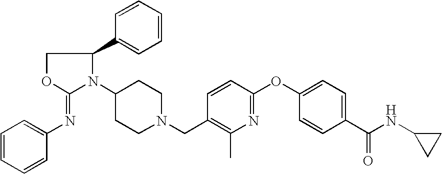

### COMPOUND 344: N-Cyclopropyl-4-[6-methyl-5-(4-{(R)-4-phenyl-2-[phenylimino]-oxazolidin-3-yl}-1-piperidin-1-ylmethyl)-pyridin-2-yloxy]-benzamide

To a solution of 4-[(R)-2-hydroxy-1-phenyl-ethylamino]-piperidine-1-carboxylic acid tert-butyl ester (346 mg, 1.08 mmol) in CH₂Cl₂ (3 mL) was added phenylisocyanate (135 mg, 1.14 mmol) and the mixture was stirred at rt for 1 h. Solvent was evaporated and the residue was purified by column chromatography on silica gel, eluted with 3:1 to 1:1 hexane-ethyl acetate to provide 4-[1-((R)-2-hydroxy-1-phenyl-ethyl)-3-phenyl-ureido]-piperidine-1-carboxylic acid tert-butyl ester (328 mg, 69%).

To a solution of 4-[1-((R)-2-hydroxy-1-phenyl-ethyl)-3-phenyl-ureido]-piperidine-1-carboxylic acid tert-butyl ester (328 mg, 0.747 mmol) in CH₂Cl₂ (6 mL) and triethylamine (113, mg, 1.12 mmol) was added methanesulfonylchloride (90 mg, 0.78 mmol) and the mixture was stirred at rt for 3 h and then heated at reflux for 1 h. Solvent was evaporated and the residue was purified by column chromatography on silica gel (4:1 to 1:1, hexane/EtOAc) to provide 4-[(R)-4-phenyl-2-phenylimino-oxazolidin-3-yl]-piperidine-1-carboxylic acid tert-butyl ester (145 mg, 46%).

Following general procedure C, the above carbamate (110 mg, 0.261 mmol) in CH₂Cl₂ (3 mL) was treated with TFA (1 mL) at rt for 1 h to give phenyl-[(R)-4-phenyl-3-piperidin-4-yl-oxazolidin-2-ylidene]-amine (71.0 mg, 85%). ¹H NMR (CDCl₃) δ 1.06-1.20 (m, 1H), 1.62-1.94 (m, 4H), 2.55 (t, 1H, J=12.0 Hz), 2.69 (t, 1H, J=12.0 Hz), 2.90 (d, 1H, J=12.0 Hz), 3.12 (d, 1H, J=12.0 Hz), 4.00 (tt, 1H, J=12.0, 3.6 Hz), 4.06 (dd, 1H, J=8.4, 5.7 Hz), 4.54 (t, 1H, J=8.4 Hz), 4.80 (dd, 1H, J=8.1, 5.4 Hz), 6.98 (t, 1H, J=7.2 Hz), 7.10 (d, 2H, J=8.1 Hz), 7.27 (t, 2H, J=7.8 Hz), 7.31-7.40 (m, 5H).

Following general procedure A, N-cyclopropyl-4-(5-formyl-6-methyl-pyridin-2-yloxy)-benzamide (40.0 mg, 0.135 mmol) was reacted with phenyl-[(R)-4-phenyl-3-piperidin-4-yl-oxazolidin-2-ylidene]-amine (36.0 mg, 0.112 mmol) in the presence of NaBH(OAc)₃ (40.0 mg, 0.179 mmol) in dichloromethane (1 mL) to provide COMPOUND 344 (35.8 mg, 53%). ¹H NMR (CDCl₃) δ 0.58-0.64 (m, 2H), 0.83-0.90 (m, 2H), 1.14-1.28 (m, 1H), 1.58 (d, 1H, J=12.3 Hz), 1.85-2.17 (m, 4H), 2.39 (s, 3H), 2.67 (d, 1H, J=11.4 Hz), 2.85-2.91 (m, 2H), 3.33 (s, 2H), 3.94 (m, 1H), 4.06 (dd, 1H, J=8.4, 5.4 Hz), 4.54 (t, 1H, J=8.4 Hz), 4.78 (dd, 1H, J=8.4, 5.4 Hz), 6.26 (br s, 1H), 6.61 (d, 1H, J=8.1 Hz), 6.98 (t, 1H, J=7.5 Hz), 7.08-7.13 (m, 4H), 7.24-7.29 (m, 2H), 7.31-7.41 (m, 5H), 7.49 (d, 1H, J=8.4 Hz), 7.74 (d, 2H, J=8.7 Hz); ¹³C NMR (CDCl₃) δ 7.15, 22.24, 23.54, 29.22, 30.95, 53.39, 53.55, 53.62, 59.07, 59.33, 73.45, 108.82, 120.34, 122.46, 123.88, 127.08, 128.31, 128.93, 129.41, 130.32, 141.42, 141.88, 148.31, 153.22, 157.02, 158.07, 161.21, 168.69; ES-MS m/z 602 (M+H). Anal. Calcd. for C₃₇H₃₉N₅O₃.0.2CH₂Cl₂.0.3C₆H₁₄: C, 72.67; H, 6.82; N, 10.86. Found: C, 73.04; H, 6.73; N, 10.91.

### EXAMPLE 345

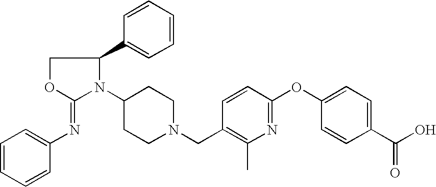

### COMPOUND 345: 4-[6-Methyl-5-(4-{(R)-4-phenyl-2-[phenylimino]-oxazolidin-3-yl}-piperidin-1-ylmethyl)-pyridin-2-yloxy]-benzoic acid

Following general procedure A, phenyl-[(R)-4-phenyl-3-piperidin-4-yl-oxazolidin-2-ylidene]-amine (see EXAMPLE 344) (36.0 mg, 0.112 mmol) was reacted with 4-(5-formyl-6-methyl-pyridin-2-yloxy)-benzoic acid methyl ester (36.6 mg, 0.135 mmol) in the presence of NaBH(OAc)₃ (40 mg, 0.18 mmol) in dichloromethane (1 mL) to provide 4-{6-methyl-5-[4-(4R)-phenyl-2-phenylimino-oxazolidin-3-yl)-piperidin-1-ylmethyl]-pyridin-2-yloxy}-benzoic acid methyl ester as a white foam (40.8 mg, 63%).

Following general procedure H, the above product (40.8 mg, 0.071 mmol) was treated with 2 N NaOH (1 mL) in methanol (1 mL) at 50° C. for 4 h to give COMPOUND 345 as a white powder (24.4 mg, 61%). ¹H NMR (CDCl₃) δ 1.31-1.43 (m, 1H), 1.65 (d, 1H, J=12.3 Hz), 2.00 (d, 1H, J=11.1 Hz), 2.14-2.24 (m, 2H), 2.37-2.41 (m, 1H), 2.40 (s, 3H), 2.96 (d, 1H, J=10.8 Hz), 3.25 (d, 1H, J=9.9 Hz), 3.52 (d, 1H, J=13.2 Hz), 3.62 (d, 1H, J=13.2 Hz), 4.06-4.16 (m, 2H), 4.55 (t, 1H, J=8.4 Hz), 4.79 (dd, 1H, J=9.0, 5.4 Hz), 6.59 (d, 1H, J=8.1 Hz), 7.00 (t, 1H, J=7.2 Hz), 7.12 (d, 4H, J=8.1 Hz), 7.21-7.30 (m, 7H), 7.59 (d, 1H, J=8.1 Hz), 8.00 (d, 2H, J=8.4 Hz); ¹³C NMR (CDCl₃) δ 22.44, 27.98, 29.75, 52.69, 52.84, 53.12, 58.34, 59.03, 73.62, 109.23, 120.05, 122.81, 123.94, 127.12, 128.98, 129.47, 132.07, 141.27, 142.89, 147.68, 153.62, 157.42, 158.78, 161.95, 169.92; ES-MS m/z 563 (M+H). Anal. Calcd. for C₃₄H₃₄N₄O₄.0.6CH₂Cl₂: C, 67.73; H, 5.78; N, 9.13. Found: C, 67.60; H, 5.77; N, 9.04.

### EXAMPLE 346

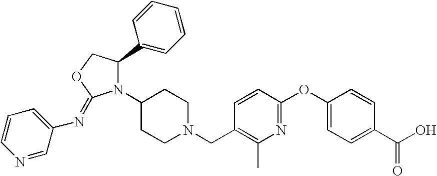

### COMPOUND 346: 4-(6-Methyl-5-{4-[(R)-4-phenyl-2-(pyridin-3-ylimino)-oxazolidin-3-yl]-piperidin-1-ylmethyl}-pyridin-2-yloxy)-benzoic acid

Following general procedure A, [(R)-4-phenyl-3-piperidin-4-yl-oxazolidin-2-ylidene]-pyridin-3-yl-amine (see EXAMPLE 343) (81.5 mg, 0.253 mmol) was reacted with 4-(5-formyl-6-methyl-pyridin-2-yloxy)-benzoic acid methyl ester (82.3 mg, 0.304 mmol) in the presence of NaBH(OAc)₃ (90.3 mg, 0.405 mmol) in CH₂Cl₂ (2.0 mL) to give 4-(6-methyl-5-{4-[4(R)-phenyl-2-(pyridin-3-ylimino)-oxazolidin-3-yl]-piperidin-1-ylmethyl}-pyridin-2-yloxy)-benzoic acid methyl ester (93.6 mg, 64%).

Following general procedure H, the above product (93.6 mg, 0.162 mmol) was treated with 1 N NaOH (1.0 mL) in MeOH (1.0 mL) at rt for 21 h to give COMPOUND 346 as a white powder (55.0 mg, 60%). ¹H NMR (CD₃OD) δ 1.64-1.76 (m, 1H), 1.85 (d, 1H, J=12.9 Hz), 2.05 (d, 1H, J=12.3 Hz), 2.28-2.41 (m, 1H), 2.47 (s, 3H), 2.64-2.79 (m, 2H), 3.17 (d, 1H, J=11.1 Hz), 3.35 (m, 1H), 3.95-4.05 (m, 1H), 3.96 (s, 2H), 4.21 (t, 1H, J=6.5 Hz), 4.74 (t, 1H, J=8.1 Hz), 5.06 (t, 1H, J=6.5 Hz), 6.81 (d, 1H, J=8.4 Hz), 7.15 (d, 2H, J=4.2 Hz), 7.35-7.45 (m, 7H), 7.65 (d, 1H, J=7.5 Hz), 7.83 (d, 1H, J=8.1 Hz), 8.08 (br s, 3H); ES-MS m/z 564 (M+H). Anal. Calcd. for C₃₃H₃₃N₅O₄.1.1CH₂Cl₂: C, 62.33; H, 5.40; N, 10.66. Found: C, 62.18; H, 5.70; N, 10.66.

### EXAMPLE 347

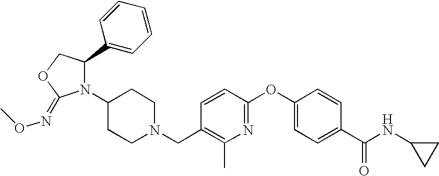

### COMPOUND 347: N-Cyclopropyl-4-{5-[4-((R)-2-methoxyimino-4-phenyl-oxazolidin-3-yl)-piperidin-1-ylmethyl]-6-methyl-pyridin-2-yloxy}-benzamide

To a solution of 4-[(R)-2-hydroxy-1-phenyl-ethylamino]-piperidine-1-carboxylic acid tert-butyl ester (620 mg, 1.94 mmol) in CH₂Cl₂ (10 mL) at 0° C. was added methoxyl-carbamic acid 4-nitro-phenylester (411 mg, 1.94 mmol) followed by DIPEA (300 mg, 2.33 mmol) and the mixture was stirred at rt for 1 h. Solvent was evaporated and the residue was purified by column chromatography on silica gel (4:1, hexane/EtOAc to 100% EtOAc) to provide 4-{1-[(R)-2-hydroxy-1-phenyl-ethyl]-3-methoxyl-ureido}-piperidine-1-carboxylic acid tert-butyl ester (597 mg, 78%).

To a solution of 4-{1-[(R)-2-hydroxy-1-phenyl-ethyl]-3-methoxyl-ureido}-piperidine-1-carboxylic acid tert-butyl ester (300 mg, 0.763 mmol) in CH₂Cl₂ (5 mL) and triethylamine (154 mg, 1.53 mmol) was added methanesulfonylchloride (105 mg, 0.916 mmol) and the mixture was stirred at rt for 1 h. Solvent was evaporated and the residue was purified by column chromatography on silica gel, eluted with 4:1 to 1:1 hexane-ethyl acetate to provide 4-[(R)-2-methoxyimino-4-phenyl-oxazolidin-3-yl]-piperidine-1-carboxylic acid tert-butyl ester (236 mg, 83%).

Following general procedure C: the above carbamate (235 mg, 0.627 mmol) in CH₂Cl₂ (3 mL) was treated with TFA (1 mL) at rt for 1 h to give (R)-4-Phenyl-3-piperidin-4-yl-oxazolidin-2-one O-methyl-oxime (171 mg, 99%). ¹H NMR (CDCl₃) δ 1.14-1.27 (m, 1H), 1.51-1.57 (m, 2H), 1.75-1.83 (m, 2H), 2.41-2.61 (m, 2H), 2.89 (d, 1H, J=12.3 Hz), 3.06 (d, 1H, J=12.0 Hz), 3.43 (m, 1H), 3.74 (s, 3H), 4.06 (dd, 1H, J=8.1, 6.6 Hz), 4.57 (t, 1H, J=8.1 Hz), 4.72 (dd, 1H, J=7.8, 6.9 Hz), 7.29-7.35 (m, 5H).

Following general procedure A, (R)-4-phenyl-3-piperidin-4-yl-oxazolidin-2-one O-methyl-oxime (55.0 mg, 0.200 mmol) reacted with N-cyclopropyl-4-(5-formyl-6-methyl-pyridin-2-yloxy)-benzamide (71.0 mg, 0.240 mmol) in the presence of NaBH(OAc)₃ (71.4 mg, 0.320 mmol) in CH₂Cl₂ (2.0 mL) to give COMPOUND 347 as a white powder (85.3 mg, 77%). ¹H NMR (CDCl₃) δ 0.58-0.63 (m, 2H), 0.79-0.88 (m, 2H), 1.22-1.41 (m, 1H), 1.51 (d, 1H, J=11.4 Hz), 1.84-2.00 (m, 4H), 2.37 (s, 3H), 2.67 (br s, 1H), 2.84-2.92 (m, 2H), 3.22-3.51 (m, 3H), 3.75 (s, 314), 4.08 (t, 1H, J=7.2 Hz), 4.59 (t, 1H, J=8.1 Hz), 4.72 (t, 1H, J=7.2 Hz), 6.29 (br s, 1H), 6.60 (d, 1H, J=8.1 Hz), 7.10 (d, 2H, J=8.7 Hz), 7.33 (m, 5H), 7.50 (d, 1H, J=7.2 Hz), 7.74 (d, 2H, J=8.4 Hz); ¹³C NMR (CDCl₃) δ 7.10, 22.2, 23.5, 28.3, 29.8, 53.2, 53.3, 53.6, 59.0, 60.3, 62.6, 74.7, 108.9, 120.4, 127.2, 127.6, 129.0, 129.2, 129.4, 130.4, 140.6, 141.6, 157.0, 157.2, 157.9, 161.3, 168.7; ES-MS m/z 556 (M+H). Anal. Calcd. for C₃₂H₃₇N₅O₄.0.8CH₂Cl₂: C, 63.17; H, 6.24; N, 11.23. Found: C, 63.37; H, 6.35; N, 11.00.

### EXAMPLE 348

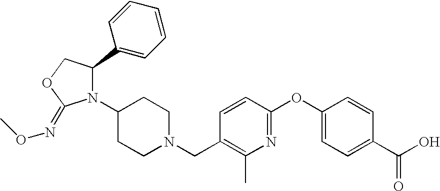

### COMPOUND 348: 4-{5-[4-((R)-2-Methoxyimino-4-phenyl-oxazolidin-3-yl)-piperidin-1-ylmethyl]-6-methyl-pyridin-2-yloxy}-benzoic acid

Following general procedure A, (R)-4-phenyl-3-piperidin-4-yl-oxazolidin-2-one O-methyl-oxime (see EXAMPLE 347) (80.0 mg, 0.291 mmol) was reacted with 4-(5-formyl-6-methyl-pyridin-2-yloxy)-benzoic acid methyl ester (94.6 mg, 0.349 mmol) in the presence of NaBH(OAc)₃ (103.9 mg, 0.466 mmol) in CH₂Cl₂ (2.0 mL) to give 4-{5-[4-(2-methoxyimino-4(R)-phenyl-oxazolidin-3-yl)-piperidin-1-ylmethyl]-6-methyl-pyridin-2-yloxy}-benzoic acid methyl ester (103.6 mg, 67%).

Following general procedure H, the above product (50.0 mg, 0.094 mmol) was treated with 1 N NaOH (0.5 mL) in MeOH (0.5 mL) at rt for 17 h to give COMPOUND 348 as a white powder (46.8 mg, 96%). ¹H NMR (CDCl₃) δ 1.57 (d, 1H, J=11.7 Hz), 1.83-2.10 (m, 2H), 2.40 (s, 3H), 2.54 (m, 2H), 2.72 (m, 1H), 3.21 (d, 1H, J=10.2 Hz), 3.47 (m, 1H), 3.75 (s, 3H), 4.00 (d, 2H, J=9.3 Hz), 4.16 (dd, 1H, J=8.1, 6.0 Hz), 4.63 (t, 1H, J=9 Hz), 4.79 (t, 1H, J=7.5 Hz), 6.76 (d, 1H, J=8.4 Hz), 7.13 (d, 2H, J=8.4 Hz), 7.27-7.34 (m, 5H), 8.03 (d, 2H, J=8.4 Hz), 8.16 (br s, 1H); ¹³C NMR (CDCl₃) δ 21.4, 23.9, 49.6, 50.7, 51.1, 54.9, 58.6, 61.4, 73.7, 108.8, 118.4, 119.4, 125.9, 126.0, 126.2, 127.9, 128.3, 130.7, 138.6, 142.9, 155.8, 156.1, 156.9, 161.4, 168.1; ES-MS m/z 517 (M+H). Anal. Calcd. for C₂₉H₃₂N₄O₅.1.3CH₂Cl₂: C, 58.04; H, 5.56; N, 8.94. Found: C, 58.24; H, 5.80; N, 8.90.

### EXAMPLE 349

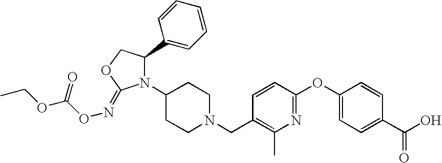

### COMPOUND 349: 4-{5-[4-(R)-2-Ethoxycarbonylmethylimino-4-phenyl-oxazolidin-3-yl)-piperidin-1-ylmethyl]-6-methyl-pyridin-2-yloxy}-benzoic acid

To a solution of 4-[(R)-2-hydroxy-1-phenyl-ethylamino]-piperidine-1-carboxylic acid tert-butyl ester (1.32 g, 4.12 mmol) in CH₂Cl₂ (7 mL) was added a solution of ethylisocyanatoacetate (532 mg, 4.12 mmol) in CH₂Cl₂ (3 mL) and the mixture was stirred at rt for 2 h. Solvent was evaporated to provide 4-[3-ethoxycarbonylmethyl-1-(2-hydroxy-1-phenyl-ethyl)-ureido]-piperidine-1-carboxylic acid tert-butyl ester (1.82 g, 99%).

To a solution of above product (1.29 g, 2.87 mmol) in CH₂Cl₂ (10 mL) and triethylamine (0.88 mL, 6.32 mmol) was added a solution of methanesulfonylchloride (493 mg, 0.78 mmol) in CH₂Cl₂ (5 mL) dropwise and the mixture was stirred at rt for 1.5 h and then heated at reflux for 1 h. Solvent was evaporated and the residue was purified by column chromatography on silica gel (1:1 to hexane-EtOAc to 100% EtOAc) to provide 4-(2-ethoxycarbonylmethylimino-4-phenyl-oxazolidin-3-yl)-piperidine-1-carboxylic acid tert-butyl ester (993 mg, 80%).

Following general procedure C: the above product (309 mg, 0.717 mmol) in CH₂Cl₂ (3 mL) was treated with TFA (1 mL) at rt for 1 h to give ((R)-4-phenyl-3-piperidin-4-yl-oxazolidin-2-ylideneamino)-acetic acid ethyl ester (237 mg, 100%). ¹H NMR (CDCl₃) δ 1.25 (t, 1H, J=7.2 Hz), 1.25-1.35 (m, 1H), 1.90-1.98 (m, 2H), 2.56 (td, 1H, J=12.8, 2.1 Hz), 2.69-2.77 (m, 1H), 2.96 (d, 1H, J=12.6 Hz), 3.18 (d, 1H, J=12.3 Hz), 3.82-3.90 (m, 1H), 3.99 (dd, 1H, J=8.4, 5.4 Hz), 4.02 (d, 1H, J=15.5 Hz), 4.05 (d, 1H, J=15.5 Hz), 4.15 (q, 2H, J=7.2 Hz), 4.47 (t, 1H, J=8.4 Hz), 4.72 (dd, 1H, J=8.1, 5.4 Hz), 6.41 (s, 1H), 7.31-7.40 (m, 5H).

Following general procedure A, ((R)-4)-phenyl-3-piperidin-4-yl-oxazolidin-2-ylideneamino)-acetic acid ethyl ester (202.0 mg, 0.610 mmol) was reacted with 4-(5-formyl-6-methyl-pyridin-2-yloxy)-benzoic acid tert-butyl ester (229.2 mg, 0.732 mmol) in the presence of NaBH(OAc)₃ (206.9 mg, 0.976 mmol) in CH₂Cl₂ (3.0 mL) to give 4-{5-[4-((R)-2-ethoxycarbonylmethylimino-4-phenyl-oxazolidin-3-yl)-piperidin-1-ylmethyl]-6-methyl-pyridin-2-yloxy}-benzoic acid tert-butyl ester (291.5 mg, 76%).

The above product (291.5 mg, 0.464 mmol) was treated with TFA (1.2 mL) in CH₂Cl₂ (2.0 mL) rt for 2 h to give COMPOUND 349 as a white powder (310.9 mg, 100%). ¹H NMR (CDCl₃) δ 1.29 (t, 3H, J=6.9 Hz), 1.67 (m, 2H), 2.18 (d, 1H, J=12.9 Hz), 2.36 (s, 3H), 2.54-2.66 (m, 1H), 2.96-3.18 (m, 2H), 3.25 (d, 1H, J=10.8 Hz), 3.54 (d, 1H, J=10.8 Hz), 4.06 (s, 2H), 4.21 (s, 2H), 4.33 (q, 2H, J=6.9 Hz), 4.55 (dd, 1H, J=8.7, 3.6 Hz), 4.84 (br s, 1H), 4.98 (t, 1H, J=9.0 Hz), 5.37 (dd, 1H, J=9.0, 3.9 Hz), 6.71 (d, 1H, J=8.1 Hz), 7.11 (d, 2H, J=8.1 Hz), 7.29-7.38 (m, 5H), 7.76 (d, 1H, J=8.1 Hz), 7.99 (d, 2H, J=8.1 Hz); ¹³C NMR (CDCl₃) δ 14.4, 22.3, 26.7, 27.8, 27.9, 44.3, 51.2, 51.3, 53.9, 56.9, 61.1, 62.6, 110.1, 115.1, 118.8, 119.0, 120.9, 126.8, 127.5, 130.4, 130.6, 132.0, 137.5, 143.9, 158.0, 158.1, 161.2, 162.2, 162.7, 162.9, 168.1, 169.0; ES-MS m/z 573 (M+H). Anal. Calcd. for C₃₂H₃₆N₄O₆.2.4CH₂Cl₂: C, 53.21; H, 5.30; N, 7.22. Found: C, 53.11; H, 4.91; N, 6.90.

### EXAMPLE 350

### Cell Fusion Assay

The assay measures the ability of a test compound to inhibit gp120 and CD4/CCR5-dependent cell-cell fusion. The assay uses two cell lines, 1) CHO-tat cell line that expresses the viral gp120 from a R5 using virus (JR-FL) and the HIV tat proteins, 2) P4-CCR5 cell line that expresses human CD4 and CCR5 on the surface and carries a β-galactosidase construct under the control of the retroviral promotor LTR. Once fusion of these two cell lines occurs, the tat protein from the CHO cell line trans-activates the reporter gene 0-galactosidase in the P4-CCR5 cell line. In a 96 well format, 1×10⁴ cells of each cell line are plated per well in the presence or absence of test compound. The cells are then incubated at 37° C., 5% CO₂ for 18-24 hours. The β-galactosidase activity in each well is measured by the addition of a luminescence substrate (Gal-Screen substrate, Applied Biosystems) and luminescence monitored with a Victor 2 plate reader (Wallac). The ability of test compounds to inhibit fusion is indicated by a decrease in β-galactosidase activity. Results are reported as the concentration of test compound required to inhibit 50% of the β-galactosidase activity in the test controls.

When tested in the assay described above, many compounds of the invention exhibited IC₅₀'s in the range of 0.01 nM to 100 nM.

### EXAMPLE 351

### Assay for Inhibition of RANTES Binding to HEK293F.CCR5 Cells

For the competition binding studies, a concentration range of antagonist was incubated for 45 minutes at room temperature in binding buffer (50 mM HEPES, 5 mM MgCl₂, 1 mM CaCl₂, 0.2% BSA pH 7.4) with 8 μg of HEK293F.CCR5 cell membrane and 50 pM ¹²⁵I-RANTES (Perkin Elmer, 81400 GBq/mmol) in Milipore GF-B filter plates. Unbound ¹²⁵I-RANTES was removed by washing with cold 50 mM HEPES, 0.5 M NaCl pH 7.4. Compounds were tested at a concentration range of 10000-0.6 nM. The 50% inhibitory concentration (IC₅₀ value) was defined as the concentration of test compound required to inhibit RANTES binding by 50% relative to untested controls.

When tested in the assay described above, the compounds of the invention exhibited IC₅₀'s in the range of 1 nM to 500 nM.

### EXAMPLE 352

### Assay for Inhibition of HIV-1 Using PBMC and R⁵

Performed as described in literature (Inhibition of T-tropic HIV strains by selective antagonization of the chemokine receptor CXCR4. 1997. D. Schols, S. Struyf, J. Van Damme, J. A. Esté, G. Henson & E. De Clercq. J. Exp. Med. 186, 1383-1388.)

The method were as follows:

PBMC from healthy donors were isolated by density gradient centrifugation and stimulated with PHA at 1 μg/ml (Sigma Chemical Co., Bornem, Belgium) for 3 days at 37° C. The activated cells (PHA-stimulated blasts) were washed three times with PBS, and viral infections were performed. The cells were seeded in 48-well plates (5×10⁵ cells per well in 200 uL culture medium) and pre-incubated for 15 min with compounds at different concentrations. Then 500 pg p24 viral Ag/well of CCR5-using viruses was added. The HIV-1 R5 strains BaL, SF-162, ADA, and JR-FL were all obtained through the Medical Research Council AIDS reagent project (Herts, UK).

HIV-infected or mock-infected PHA-stimulated blasts were then further cultured in the presence of 25 U/ml of IL-2 and supernatant was collected at days 8-10, and HIV-1 core antigen in the culture supernatant was analyzed by the p24 Ag ELISA kit from DuPont-Merck Pharmaceutical Co. (Wilmington, Del.).

When tested in the assay described above, many compounds of the invention exhibited IC₅₀'s in the range of 0.01 nM to 50 μM.

### EXAMPLE 353

### CCR4-mediated Intracellular Calcium Mobilization Assay

The assay measures the ability of a test compound to stimulate an increase in intracellular calcium through its binding to the chemokine receptor CCR4. The HEK293F cells used in this assay are stably transfected with the human CCR4 receptor and the chimeric G protein Gqi5. The cells were loaded with the calcium indicator Fluo-4-AM (Molecular Probes Inc.). The loaded cells were then washed and incubated in HBSS containing 20 mM HEPES, 0.2% BSA, 2.5 mM probenecid, pH 7.4. The cells were plated on a 96-well plate and pre-incubated for 15 min at 37° C. The plate was then transferred to the FLEXstation fluorescent plate reader (Molecular Devices) where addition of a concentration range of test compound was performed and fluorescence was immediately monitored. Results are reported as the concentration of test compound required to induce 50% of the maximal intracellular calcium release obtained with that compound (EC₅₀), and the percentage of maximal TARC (specific CCR4 chemokine) response obtained at the highest concentration of test compound tested.

When tested in the assay described above, some of the compounds of the invention exhibited EC₅₀'s in the range of 1 nM to 5000 nM.

What is claimed is:

1. A compound or pharmaceutically acceptable salt thereof, having the formula (1) 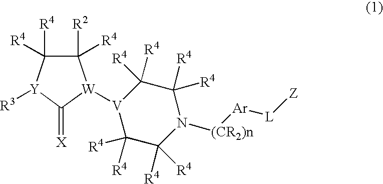
wherein:
V is N or C(R);
W is N or C(R);
X is O, S, NR, N-aryl, N-heteroaryl, N-heterocyclyl, NOR, NCOR, N(CH₂)ₘCOOR, N(CH₂)ₘCONHR, NS(O₂)R, NCN, NNO₂, or CRNO₂, wherein m is 0-3;
Y is O, S, N or C(R);
Z may be absent, H or an optionally substituted alkyl, OR, COOR, C(O)NR₂, carbocyclyl, heterocyclyl, aryl, or heteroaryl;
Ar is an optionally substituted carbocyclyl, heterocyclyl, aryl, or heteroaryl, wherein each of the carbocyclyl and heterocyclyl contains an aryl or heteroaryl ring;
L is absent if Z is absent, or L is a linker between Ar and Z, wherein L is a bond, O, S, N(R), S(O), S(O₂), S(O₂)N(R), C(O), C(O)N(R), N(R)C(O)N(R), N═N, optionally substituted aliphatic C₁₋₆ hydrocarbyl residue optionally containing one or more heteroatoms, or combinations thereof;
R² is an optionally substituted alkyl, alkenyl, alkynyl, carbocyclyl, heterocyclyl, aryl or heteroaryl;
R³ is absent when Y is O or S; or, when Y is N or C(R), R³ is H, NR₂, C(O)NHOR, C(O)N(R)OR, C(O)NR₂, C(O)R, C(O)OR, OR, or an optionally substituted alkyl, carbocyclyl, heterocyclyl, aryl or heteroaryl;
each R and R⁴ is independently H or C₁₋₆ alkyl; and
n is 1-3.

2. The compound of claim 1, wherein V is CH.

3. The compound of claim 1, wherein W is N.

4. The compound of claim 1, wherein X is O, S, N-pyridyl, N-phenyl, NOR or NCH₂COOR.

5. The compound of claim 1, wherein Y is N, O or C(R).

6. The compound of claim 1, wherein Z is an optionally substituted alkyl, alkoxy, cycloalkyl, phenyl, benzyl, pyridinyl, pyrimidinyl, tetrahydropyranyl, piperidinyl, piperazinyl, dihydroisoindolonyl, dihydroindolonyl, or benzodioxolyl.

7. The compound of claim 6, wherein Z is unsubstituted or is optionally substituted with one or more alkyl, carbocyclyl, heterocyclyl, aryl, heteroaryl, alkenyl, alkynyl, halogen, CN, CHO, CF₃, OCF₃, NO₂, R⁵, NRR⁵, OR⁵, N(R)C(O)R⁵, N(R)C(O)CF₃, N(R)S(O₂)R⁵, N(R)S(O₂)NRR⁵, N(R)C(O)NRR⁵, SO₃R, C(O)NRR⁵, C(O)N(OC₁₋₆ alkyl)R, C(O)R⁵, OS(O₂)R, OC(O)NRR⁵, OC(O)R⁵, COOR⁵, SR⁵, S(O)R⁵, S(O₂)R⁵, C(R)═NOH, C(R)═NO(C₁₋₆ alkyl), C(R)═N(C₁₋₆ alkyl), (EC₁₋₄ linker)R⁵, (C₁₋₄ linker)Cl, (C₁₋₄ linker)CN, (C₁₋₄ linker)CF₃, (C₁₋₄ linker)OCF₃, (C₁₋₄ linker)NRR⁵, (C₁₋₄ linker)OR⁵, (C₁₋₄ linker)N(R)C(O)R⁵, (C₁₋₄ linker)N(R)C(O)CF₃, (C₁₋₄ linker)N(R)S(O₂)R⁵, (C₁₋₄ linker)N(R)S(O₂)NRR⁵, (C₁₋₄ linker)N(R)C(O)NRR⁵, (C₁₋₄ linker)SO₃R, (C₁₋₄ linker)C(O)NRR⁵, (C₁₋₄ linker)C(O)N(OC₁₋₆ alkyl)R, (C₁₋₄ linker)C(O)R⁵, (C₁₋₄ linker)OS(O₂)R, (C₁₋₄ linker)OC(O)NRR⁵, (C₁₋₄ linker)OC(O)R⁵, (C₁₋₄ linker)COOR⁵, (C₁₋₄ linker)SR⁵, (C₁₋₄ linker)S(O)R⁵, (C₁₋₄ linker)S(O₂)R⁵, (C₁₋₄ linker)C(R)═NOH, (C₁₋₄ linker)C(R)═NO(C₁₋₆ alkyl), (EC₁₋₄ linker)CN, (EC₁₋₄ linker)CF₃, (EC₁₋₄ linker)NRR⁵, (EC₁₋₄ linker)OR⁵, (EC₁₋₄ linker)N(R)C(O)R⁵, (EC₁₋₄ linker)N(R)C(O)CF₃, (EC₁₋₄ linker)N(R)S(O₂)R⁵, (EC₁₋₄ linker)N(R)S(O₂)NRR⁵, (EC₁₋₄ linker)N(R)C(O)NRR⁵, (EC₁₋₄ linker)C(O)NRR⁵, (EC₁₋₄ linker)R⁵, (EC₁₋₄ linker)C(O)N(OC₁₋₆ alkyl)R, (EC₁₋₄ linker)C(O)R⁵, (EC₁₋₄ linker)OS(O₂)R, (EC₁₋₄ linker)OC(O)NRR⁵, (EC₁₋₄ linker)OC(O)R⁵, (EC₁₋₄ linker)COOR⁵, (EC₁₋₄ linker)SR⁵, (EC₁₋₄ linker)S(O)R⁵, (EC₁₋₄ linker)S(O₂)R⁵, (EC₁₋₄ linker)C(R)═NOH, (EC₁₋₄ linker)C(R)═NO(C₁₋₆ alkyl), or (EC₁₋₄ linker)C(R)═N(C₁₋₆ alkyl),
wherein E is O, S, or N(R),
wherein R⁵ is H or alkyl, carbocyclyl, heterocyclyl, aryl or heteroaryl, each of which is optionally substituted by one or more of C₁₋₆ alkyl, OR, NR₂, NR(C₁₋₆ alkyl), halogen, CN, CF₃, OCF₃, N(R)C(O)(C₁₋₆ alkyl), (C₁₋₄ linker)COOR, (C₁₋₄ linker)CONHR, C(O)NH₂, C(O)NR(C₁₋₆ alkyl), C(O)N(C₁₋₄ alkyl)₂, C(O)R, COOR, OC(O)R, SR, S(Oₚ)NH₂, S(Oₚ)NR(C₁₋₆ alkyl), N(R)S(O)ₚ(C₁₋₆ alkyl) or SOₚ(C₁₋₄ alkyl) where p is 1 or 2;
wherein C₁₋₄ linker is alkyl, alkenyl or alkynyl.

8. The compound of claim 7, wherein Z is unsubstituted or is optionally substituted with one or two alkyl, CN, halogen, tetrazolyl, OH, COOH, COCOOH, C(O)NH₂, CH═NOH, NHSO₂NR₂, NHSO₂NHR, NH₂, NHCOR, SO₃H, OR, C(O)NHR, C(O)NHOR, C(O)NR₂, NHSO₂R, OC(O)R, (C₁₋₄ linker)COOH, (C₁₋₄ linker)C(O)NHR, (C₁₋₄ linker)C(O)NHOR, (C₁₋₄ linker)OH, (C₁₋₄ linker)NHSO₂NR₂, (C₁₋₄ linker)NHSO₂R, (C)₄ linker)OC(O)R, NH(C₁₋₄ linker)COOH, (C₁₋₄ linker)NH₂, S(C₁₋₄ linker)C(O)NHR, S(C₁₋₄ linker)COOH, S(C₁₋₄ linker)C(O)NHOR, O(C₁₋₄ linker)C(O)NHR, O(C₁₋₄ linker)COOH or O(C₁₋₄ linker)C(O)NHOR; wherein C₁₋₄ linker is alkyl, alkenyl or alkynyl.

9. The compound of claim 1, wherein Ar is selected from the group consisting of phenyl, quinolyl, tetrahydroquinolyl, dihydroisoindolyl, thiazolyl, pyrimidinyl, pyridyl, benzimidazolyl, imidazolyl, pyrrolyl, thienyl, benzofuranylyl, indanonyl, pyrazolyl, benzo[1,3]dioxolyl, pyranyl, imidazo[1,2-a]pyridinyl, spirobenzodioxolecyclohexyl, and dihydro-isoindolonyl, and wherein Ar is optionally substituted.

10. The compound of claim 9, wherein Ar is an optionally substituted phenyl, quinolyl, tetrahydroquinolyl, dihydroisoindolyl, thiazolyl, pyrimidinyl, pyridyl, pyrazolyl, benzo[1,3]dioxolyl, imidazo[1,2-a]pyridinyl, spirobenzodioxolecyclohexyl, or dihydro-isoindolonyl.

11. The compound of claim 1, wherein Ar is unsubstituted or is optionally substituted with one or more of alkyl, carbocyclyl, heterocyclyl, aryl, heteroaryl, alkenyl, alkynyl, R⁵, OR⁵, NHR⁵, N(R⁵)₂, halogen, CN, CF₃, OCF₃, N(R)C(O)(R⁵), C(O)NRR⁵, C(O)N(R⁵)₂, C(O)R⁵, C(O)OR⁵, OC(O)R⁵, SR⁵, S(O)ₚR⁵, S(O)ₚNRR⁵, or N(R)S(O)ₚR⁵;
wherein R⁵ is H or alkyl, carbocyclyl, heterocyclyl, aryl or heteroaryl ring, each of which is optionally substituted by one or more of C₁₋₆ alkyl, OR, NR₂, NR(C₁₋₆ alkyl), halogen, CN, CF₃, OCF₃, N(R)C(O)(C₁₋₆ alkyl), (C₁₋₄ linker)COOR, (C₁₋₄ linker)CONHR, C(O)NH₂, C(O)NR(C₁₋₆ alkyl), C(O)N(C₁₋₆ alkyl)₂, C(O)R, COOR, OC(O)R, SR, S(Oₚ)NH₂, S(Oₚ)NR(C₁₋₆ alkyl), N(R)S(O)ₚ(C₁₋₆ alkyl) or SOₚ(C₁₋₆ alkyl) where p is 1 or 2.

12. The compound of claim 11, wherein Ar is unsubstituted or is optionally substituted with one or two C₁₋₆ alkyl, OR, CN, or halogen.

13. The compound of claim 1, wherein L is absent, a bond, CH(R), C(R₂), O, N(R), S, S(O), S(O₂), S(O₂)NH, NHC(O)NH, C(O), N(R)C(O), N(R)S(Oₚ), N(R)C(O)N(R), C(O)N(R), OC(O)N(R), OC(O), C(R)═C(R), C≡C, C(R)═N, N═C(R), N═N, (C₁₋₄ linker)O, (C₁₋₄ linker)N(R), (C₁₋₄ linker)S, (C₁₋₄ linker)S(Oₚ), (C₁₋₄ linker)C(O), (C₁₋₄ linker)N(R)C(O), (C₁₋₄ linker)N(R)S(Oₚ), (C₁₋₄ linker)N(R)C(O)N(R), (C₁₋₄ linker)C(O)N(R), (C₁₋₄ linker)OC(O)N(R), (C₁₋₄ linker)OC(O), (C₁₋₄ linker)N═C(R), (C₁₋₄ linker)N═N, or (C₁₋₄ linker)C(R)═N where p is 1 or 2,
wherein the C₁₋₄ linker is alkyl, alkenyl or alkynyl.

14. The compound of claim 13, wherein L is a bond, O, CH₂, CHMe, CMe₂, NMe, S, NH, C(O), C(O)NH, S(O₂)NH, NHC(O)NH, or (C₁₋₄ linker)NHC(O)NH.

15. The compound of claim 1, wherein R² is an optionally substituted alkyl, alkenyl, alkynyl, phenyl, thienyl, or pyridyl.

16. The compound of claim 1, wherein R² is unsubstituted or is optionally substituted with 1-4 substituents selected from the group consisting of alkyl, alkenyl, alkynyl, OR⁵, NHR⁵, N(R⁵)₂, halogen, CN, NO₂, CF₃, OCF₃, N(R)C(O)(R⁵), C(O)NRR⁵, C(O)N(R⁵)2, C(O)R⁵, C(O)OR⁵, OC(O)R⁵, SR⁵, S(O)ₚR⁵, S(O)ₚNRR⁵, and N(R)S(O)ₚR⁵ where p is 1 or 2;
wherein R⁵ is H or alkyl, carbocyclyl, heterocyclyl, aryl or heteroaryl ring, each of which is optionally substituted by one or more of C₁₋₆ alkyl, OR, NR₂, NR(C₁₋₆ alkyl), halogen, CN, CF₃, OCF₃, N(R)C(O)(C₁₋₆ alkyl), (C₁₋₄ linker)COOR, (C₁₋₄ linker)CONHR, C(O)NH₂, C(O)NR(C₁₋₆ alkyl), C(O)N(C₁₋₆ alkyl)₂, C(O)R, COOR, OC(O)R, SR, S(Oₚ)NH₂, S(Oₚ)NR(C₁₋₆ alkyl), N(R)S(O)ₚ(C₁₋₆ alkyl) or SOₚ(C₁₋₆ alkyl) where p is 1 or 2.

17. The compound of claim 16, wherein R² is unsubstituted or is optionally substituted with 1-2 C₁₋₆ alkyl or halo.

18. The compound of claim 1, wherein R³ is H, NR₂, C(O)NHOR, C(O)N(R)OR, C(O)NR₂, C(O)R, C(O)OR, OR, cyclopropyl, cyclobutyl, cyclopentyl, cyclohexyl, cycloheptyl, tetrahydropyranyl, tetrahydrothiopyranyl, tetrahydrofuranyl, morpholinyl, pyridyl, piperidinyl, imidazolyl, furanyl, tetrazolyl, pyrimidinyl, piperazinyl, thiazolyl, thienyl, C₁₋₆ alkyl, [1,3,4]-oxadiazolyl, bicyclo[4.2.0]octa-1,3,5-triene, oxa-bicyclo[3.2.1]octyl, dioxy-hexahydro-1-λ⁶-thiopyranyl or a phenyl that is optionally fused to a 5-6 membered heterocyclic ring, wherein each R³ may be optionally substituted.

19. The compound of claim 18, wherein R³ is H, NR₂, C(O)NHOR, C(O)N(R)OR, C(O)NR₂, C(O)R, C(O)OR, OR or an optionally substituted C₁₋₆ alkyl, phenyl, pyrimidinyl, piperazinyl, pyridyl, thiazolyl, thienyl, cyclopropyl, cyclopentyl, cyclohexyl, piperidinyl, tetrazole, tetrahydropyranyl, tetrahydrothiopyranyl, tetrahydrofuranyl, dioxy-hexahydro-1-λ⁶-thiopyranyl, or oxa-bicyclo[3.2.1]oct-3-yl.

20. The compound of claim 1, wherein R³ is unsubstituted or is optionally substituted with alkyl, aryl, heteroaryl, heterocyclic ring, alkenyl, alkynyl, halogen, CN, CF₃, OCF₃, NO₂, R⁵, NRR⁵, OR⁵, N(R)C(O)R⁵, N(R)C(O)CF₃, N(R)S(O₂)R⁵, N(R)C(O)NR₂, C(O)NRR⁵, C(O)N(OC₁₋₆ alkyl)R, C(O)R, OS(O₂)R, OC(O)NR₂, OC(O)R⁵, COOR⁵, SR⁵, S(O)R⁵, S(O₂)R⁵, (C₁₋₄ linker)R⁵, (C₁₋₄ linker)NHC(O)R, (C₁₋₄ linker)C(O)NHR or (C₁₋₄ linker)C(O)OR;
wherein the C₁₋₄ linker is alkyl, alkenyl or alkynyl;
wherein R⁵ is H or alkyl, carbocyclyl, heterocyclyl, aryl or heteroaryl ring, each of which is optionally substituted by one or more of C₁₋₆ alkyl, OR, NR₂, NR(C₁₋₆ alkyl), halogen, CN, CF₃, OCF₃, N(R)C(O)(C₁₋₆ alkyl), (C₁₋₄ linker)COOR, (C₁₋₄ linker)CONHR, C(O)NH₂, C(O)NR(C₁₋₆ alkyl), C(O)N(C₁₋₆ alkyl)₂, C(O)R, COOR, OC(O)R, SR, S(Oₚ)NH₂, S(Oₚ)NR(C₁₋₆ alkyl), N(R)S(O)ₚ(C₁₋₆ alkyl) or SOₚ(C₁₋₆ alkyl) where p is 1 or 2.

21. The compound of claim 20, wherein R³ is unsubstituted or is optionally substituted with halogen, OR, COOR, alkyl, carbocyclyl, heterocyclyl, aryl, or heteroaryl, wherein each substituent may be optionally substituted.

22. The compound of claim 1, wherein each R⁴ is H.

23. The compound of claim 1, wherein n is 1.

24. The compound of claim 1, wherein
V is CH;
W is N;
X is O;
Y is N, O or C(R), wherein R is H or C₁₋₆ alkyl;
Z is an optionally substituted alkyl, alkoxy, cycloalkyl, phenyl, benzyl, pyridinyl, pyrimidinyl, tetrahydropyranyl, piperidinyl, piperazinyl, dihydroisoindolonyl, dihydroindolonyl, or benzodioxolyl;
Ar is an optionally substituted phenyl, quinolyl, tetrahydroquinolyl, dihydroisoindolyl, thiazolyl, pyrimidinyl, pyridyl, pyrazolyl, benzo[1,3]dioxolyl, imidazo[1,2-a]pyridinyl, spirobenzodioxolecyclohexyl, or dihydro-isoindolonyl, wherein Ar is optionally substituted with one or two C₁₋₆ alkyl, OR, CN, or halogen;
L is a bond, O, CH₂, CHMe, CMe₂, NMe, S, NH, C(O), C(O)NH, S(O₂)NH, NHC(O)NH, or (C₁₋₄ linker)NHC(O)NH, wherein C₁₋₄ linker is alkyl, alkenyl or alkynyl;
R³ is H or an optionally substituted C₁₋₆ alkyl, NR₂, C(O)NHOR, C(O)N(R)OR, C(O)NR₂, C(O)R, C(O)OR, OR, phenyl, pyrimidinyl, piperazinyl, pyridyl, thiazolyl, thienyl, cyclopropyl, cyclopentyl, cyclohexyl, piperidinyl, tetrazole, tetrahydropyranyl, tetrahydrothiopyranyl, tetrahydrofuranyl, dioxy-hexahydro-1-λ⁶-thiopyranyl, or oxa-bicyclo[3.2.1]oct-3-yl, wherein R³ is optionally substituted with halogen, OR, COOR, alkyl, carbocyclyl, heterocyclyl, aryl, or heteroaryl, wherein each substituent may be optionally substituted;
R² is alkyl, alkenyl or alkynyl, phenyl, thienyl or pyridyl substituted with one or two halogen or alkyl;
R⁴ is H; and
n is 1.

25. The compound of claim 1, selected from the compounds in Examples 1-349.

26. A pharmaceutical composition comprising the compound of claim 1 and a pharmaceutically acceptable carrier.

27. A method for treating a CCR4- or CCR5-mediated disease comprising contacting the compound of claim 1 or a pharmaceutical composition thereof in a system or a subject, thereby treating said CCR4- or CCR5-mediated disease.

28. The method of claim 27, wherein said system is a cell, tissue or organ, and said subject is human or animal.

29. The method of claim 27, wherein said CCR4- or CCR5-mediated disease is allergic inflammatory conditions, asthma, HIV, an inflammatory demyelinating disease of the central nervous system, an autoimmune disease, multiple sclerosis, experimental autoimmune encephalomyelitis, psoriatic or rheumatoid arthritis, intestinal inflammation, allograft rejection, asthma, cardiovascular disease, atherosclerosis, allergic disease, allergic rhinitis, dermatitis, conjunctivitis, hypersensitivity lung disease, hypersensitivity pneumonitis, eosinophilic pneumonia, delayed-type hypersensitivity, interstitial lung disease (ILD), idiopathic pulmonary fibrosis, ILD associated with rheumatoid arthritis, systemic lupus erythematosus, ankylosing spondylitis, systemic sclerosis, Sjogren's syndrome, polymyositis, dermatomyositis, systemic anaphylaxis, myastenia gravis, juvenile onset diabetes, glomerulonephritis, autoimmune thyroiditis, graft rejection, allograft rejection, graft-versus-host disease, inflammatory bowel disease, Crohn's disease, ulcerative colitis, spondyloarthropathy, scleroderma; psoriasis, inflammatory dermatosis, dermatitis, eczema, acute dermatitis, atopic dermatitis, allergic contact dermatitis, urticaria, vasculitis, eosinphilic myotis, eosiniphilic fasciitis, tumor or cancer.

30. The method of claim 29, wherein said CCR5-mediated disease is HIV.
